(19) United States
(12) Patent Application Publication
(10) Pub. No.: US 2023/0091510 A1
(43) Pub. Date: Mar. 23, 2023

(54) ANTIBODY DRUG CONJUGATES HAVING LINKERS COMPRISING HYDROPHILIC GROUPS

(71) Applicant: Novartis AG, Basel (CH)

(72) Inventors: Zhuoliang CHEN, Belmont, MA (US); Katsumasa NAKAJIMA, Winchester, MA (US); Matthew T. BURGER, Belmont, MA (US); Joseph Anthony D'ALESSIO, Boston, MA (US); Eric MCNEILL, Arlington, MA (US); Mark G, PALMERO, Ridge, NH (US); Bing YU, Belmont, MA (US); Qiang ZHANG, East Brunswick, NJ (US)

(21) Appl. No.: 17/612,280

(22) PCT Filed: May 19, 2020
(86) PCT No.: PCT/US2020/033648

§ 371 Date: Nov. 18, 2021

Related U.S. Application Data
(60) Provisional application No. 62/850,094, filed on May 20, 2019.

(51) Int. Cl. A61K 47/68; A61K 47/54; A61K 47/60; A61K 47/61; A61P 35/00; C07K 16/28; C07K 5/062

(52) U.S. Cl. CPC A61K 47/6855; A61K 47/548; A61K 47/549; A61K 47/60; C07K 5/06052; A61K 47/6803; A61K 47/6889; A61P 35/00; C07K 16/28; A61K 47/61

(57) ABSTRACT

Provided herein are linkers, linker-drug groups and anti-body-drug conjugates comprising hydrophilic groups.

### RELATED APPLICATION

This application claims the benefit of and priority to U.S. Provisional Application No. 62/850,094, filed May 20, 2019, the contents of which is hereby incorporated by reference in its entirety.

### FIELD OF THE INVENTION

The invention provides linkers for the improvement in the solubility of antibody drug conjugates (ADCs) which comprise one or more hydrophobic drug compounds.

### BACKGROUND OF THE INVENTION

One aspect in the design of antibody drug conjugates (ADCs) is the design of the chemical linker, which links the drug moiety to the targeting moiety. Typically, an ADC uses a hydrophobic drug moiety, however when such drug moieties are used in combination with a relatively hydrophobic linker, solubility issues can arise which can affect the biocompatibility and pharmaceutical efficacy of the ADC.

Linker strategies have been reported to attempt to overcome these challenges, in particular the design of hydrophilic linkers incorporating polyethylene glycol (see R. P. Lyon, T. D. Bovee, S. O. Doronina, P. J. Burke, J. H. Hunter, H. D. Neff-LaFord, M. Jonas, M. E. Anderson, J. R. Setter, P. D. Senter, Nat. Biotechnol., 2015, 33, 733-735, and WO2015057699), linkers incorporating sulfonates (R. Y. Zhao, S. D. Wilhelm, C. Audette, G. Jones, B. A. Leece, A. C. Lazar, V. S. Goldmacher, R. Singh, Y. Kovtun, W. C. Widdison, J. M. Lambert, R. V. J. Chari, J. Med. Chem., 2011, 54, 3606-3623) and linkers having a carbohydrate backbone (F. S. Ekholm, H. Pynnönen, A. Vilkman, V. Pitkänen, J. Helin, J. Saarinen, T. Satomaa, ChemMedChem., 2016, 11(22):2501-2505). However, there remains a need for antibody drug conjugate formats that allow for the targeted delivery of hydrophobic drugs with improved pharmacokinetic and pharmacodynamic properties.

### SUMMARY OF THE INVENTION

The invention provides linkers for the use in improving the solubility of Linker-Drug conjugates in which such conjugates comprise one or more hydrophobic drug compounds, wherein the linkers comprise one or more hydrophilic groups. Various embodiments of the invention are described herein.

The invention further provides linkers for the use in improving the solubility of antibody drug conjugate (ADC) in which the ADC comprises one or more hydrophobic drug compounds, wherein the linkers comprise one or more hydrophilic groups. Various embodiments of the invention are described herein.

In one embodiment, disclosed herein are linkers which comprise one or more self immolate groups, wherein the one or more self immolate groups are each substituted with one or more hydrophilic moieties.

In one embodiment, disclosed herein are Linker-Drug groups wherein the linker comprises one or more self immolate groups coupled to the Drug, and wherein the one or more self immolate groups are each substituted with one or more hydrophilic moieties.

In one embodiment, disclosed herein are antibody drug conjugates comprising one or more Linker-Drug groups, wherein the linker comprises one or more self immolate groups coupled to the Drug, and wherein the one or more self immolate groups are each substituted with one or more hydrophilic moieties.

In one embodiment is a Linker-Drug group of Formula (I), or pharmaceutically acceptable salt thereof:

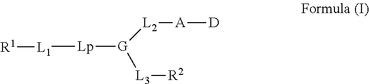

wherein:
R¹ is a reactive group;
L₁ is a bridging spacer;
Lp is a bivalent peptide spacer;
G-L₂-A is a self-immolative spacer;
R² is a hydrophilic moiety;
L₂ is a bond, a methylene, a neopentylene or a C₂-C₃alkenylene;
A is a bond, —OC(═O)—*,

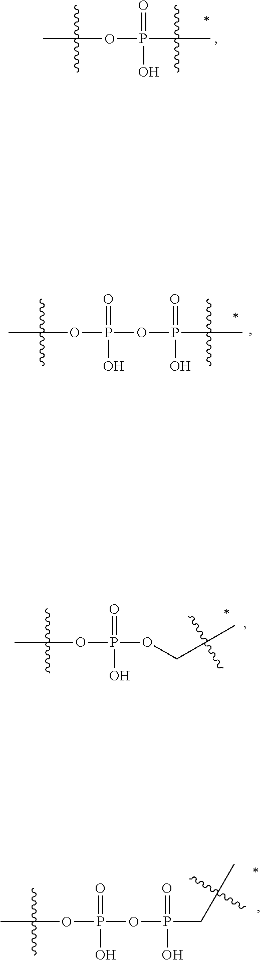 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
and
D is a Drug moiety comprising an N or an O, wherein D is connected to A via a direct bond from A to the N or the O of the Drug moiety.

In an embodiment of the Linker-Drug group of Formula (I), is a Linker-Drug group of Formula (II), or pharmaceutically acceptable salt thereof:

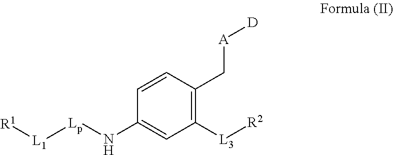

wherein:
R¹ is a reactive group;
L₁ is a bridging spacer;
Lp is a bivalent peptide linker comprising one to four amino acid residues;
R² is a hydrophilic moiety;
A is a bond, —OC(═O)—*,

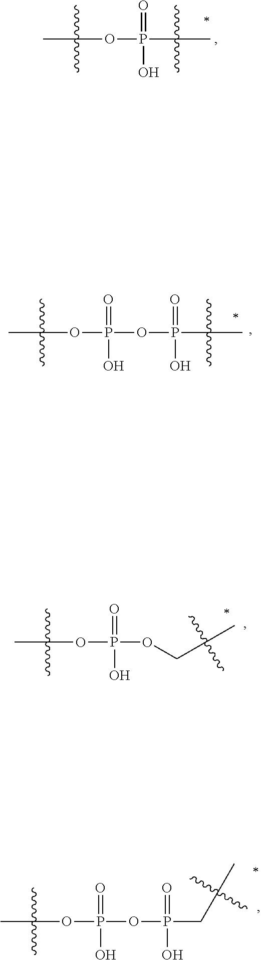 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
and
D is a Drug moiety comprising an N or an O, wherein D is connected to A via a direct bond from A to the N or the O of the Drug moiety.

In one embodiment is an Antibody Drug Conjugate of Formula (III):

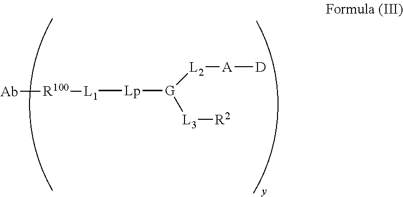

wherein:
Ab is an antibody or fragment thereof;
R¹⁰⁰ is a coupling group;
L₁ is a bridging spacer;
Lp is a bivalent peptide linker;
G-L₂-A is a self-immolative spacer;
R² is a hydrophilic moiety;
L₂ is a bond, a methylene, a neopentylene or a C₂-C₃alkenylene;
A is a bond, —OC(═O)—*,

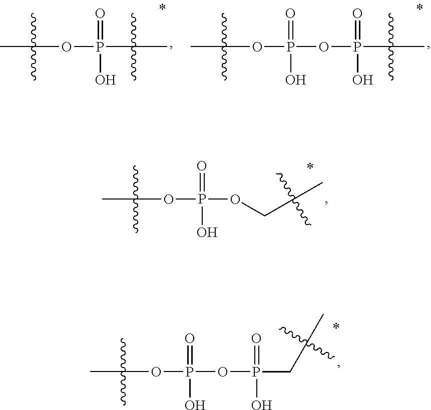 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.

In an embodiment of the Antibody Drug Conjugate of Formula (III), is an Antibody Drug Conjugate of Formula (IV):

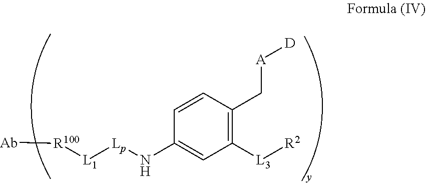

wherein:
Ab is an antibody or fragment thereof;
R¹⁰⁰ is a coupling group;
L₁ is a bridging spacer;
Lp is a bivalent peptide linker comprising one to four amino acid residues;
R² is a hydrophilic moiety;
A is a bond, —OC(═O)—*,

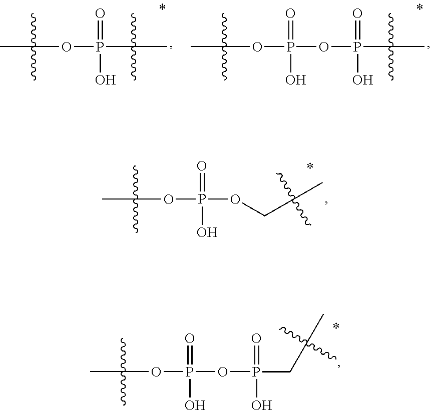 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.

Another aspect of the invention are linkers having the structure of Formula (V),

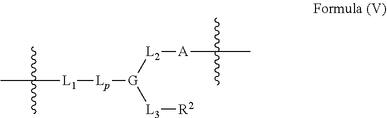

wherein
L₁ is a bridging spacer;
Lp is a bivalent peptide spacer;
G-L₂-A is a self-immolative spacer;
R² is a hydrophilic moiety;
L₂ is a bond, a methylene, a neopentylene or a C₂-C₃alkenylene;
A is a bond, **—OC(═O)—,

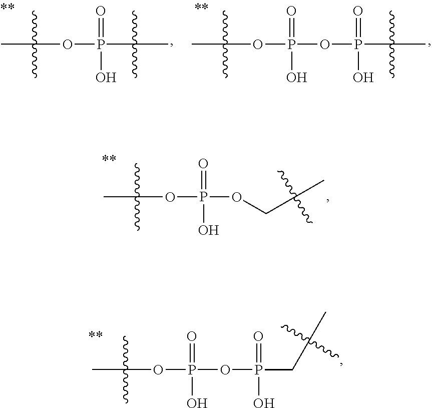 [SMILES: *OP(*)(=O)O ; *OP(=O)(O)OP(*)(=O)O ; *COP(=O)(O)O* ; *CP(=O)(O)OP(=O)(O)O*]

**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂,
and
L₃ is a spacer moiety.

In an embodiment of the of the linker Formula (V) is a linker having the structure of Formula (VI),

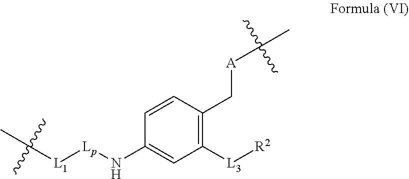

wherein
L₁ is a bridging spacer;
Lp is a bivalent peptide spacer;
R² is a hydrophilic moiety;
A is a bond, —OC(═O)—,

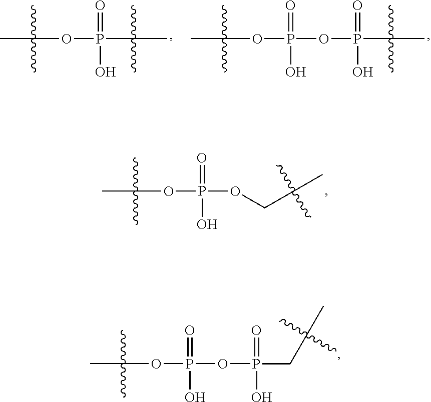 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl,
and
L₃ is a spacer moiety.

The linkers described herein, which comprise a hydrophilic moiety, contribute to the overall hydrophilicity of Antibody-Drug Conjugates (ADCs) and improve aqueous solubility of the ADC. The linkers described herein also unexpectedly reduce ADC aggregation and improve pharmacokinetic and pharmacodynamic properties of ADCs. In addition, the hydrophilic linkers described herein allow for improved aqueous solubility of the Linker-Drug group described herein thereby allowing for improved antibody conjugation to the Linker-Drug group, which improves the purification and overall synthetic yields of ADCs, in particular ADCs which comprise hydrophobic drug moieties.

### BRIEF DESCRIPTION OF THE DRAWINGS

FIG. 1 Line graphs of cellular activity of antibody drug conjugates titrated across selected cell lines A: HT-29 PCAD+; B: FaDu; C: HCC70; D:HT-29; and E: HCC1954.

FIG. 2 Line graphs of caspase-3/7 activity of antibody drug conjugates titrated across HCC1954 cell line after A: 24 hours and B: 48 hours.

FIG. 3 Efficacy and tolerability of PCAD-ADCs and the hulgG1 isotype-matched control ADCs in the HCC70 human TNBC xenograft model in SCID-beige female mice. A) Antitumor response; B) and C), changes in body weights compared to weight at start of treatment. Data are presented as means±SEM. * p<0.05, compared with untreated group (one way ANOVA with post hoc Dunnett's test) on day 20.

### DETAILED DESCRIPTION OF THE INVENTION

Various enumerated embodiments of the invention are described herein. It will be recognized that features specified in each embodiment may be combined with other specified features to provide further embodiments of the present invention.

Throughout the text of this application, should there be a discrepancy between the text of the specification (e.g., Table 3) and the sequence listing, the text of the specification shall prevail.

### Definitions

The term “alkyl”, as used herein, refers to a straight or branched hydrocarbon chain radical consisting solely of carbon and hydrogen atoms, containing no unsaturation. The term “C₁-C₆alkyl”, as used herein, refers to a straight or branched hydrocarbon chain radical consisting solely of carbon and hydrogen atoms, containing no unsaturation, having from one to six carbon atoms, and which is attached to the rest of the molecule by a single bond. Non-limiting examples of “C₁-C₆alkyl” groups include methyl (a C₁alkyl), ethyl (a C₂alkyl), 1-methylethyl (a C₃alkyl), n-propyl (a C₃alkyl), isopropyl (a C₃alkyl), n-butyl (a C₄alkyl), isobutyl (a C₄alkyl), sec-butyl (a C₄alkyl), tert-butyl (a C₄alkyl), n-pentyl (a C₅alkyl), isopentyl (a C₅alkyl), neopentyl (a C₅alkyl) and hexyl (a C₆alkyl).

The term “alkenyl”, as used herein, refers to a straight or branched hydrocarbon chain radical group consisting solely of carbon and hydrogen atoms, containing at least one double bond. The term “C₂-C₆alkenyl”, as used herein, refers to a straight or branched hydrocarbon chain radical group consisting solely of carbon and hydrogen atoms, containing at least one double bond, having from two to six carbon atoms, which is attached to the rest of the molecule by a single bond. Non-limiting examples of “C₂-C₆alkenyl” groups include ethenyl (a C₂alkenyl), prop-1-enyl (a C₃alkenyl), but-1-enyl (a C₄alkenyl), pent-1-enyl (a C₅alkenyl), pent-4-enyl (a C₅alkenyl), penta-1,4-dienyl (a C₅alkenyl), hexa-1-enyl (a C₆alkenyl), hexa-2-enyl (a C₆alkenyl), hexa-3-enyl (a C₆alkenyl), hexa-1-,4-dienyl (a C₆alkenyl), hexa-1-,5-dienyl (a C₆alkenyl) and hexa-2-,4-dienyl (a C₆alkenyl). The term “C₂-C₃alkenyl”, as used herein, refers to a straight or branched hydrocarbon chain radical group consisting solely of carbon and hydrogen atoms, containing at least one double bond, having from two to three carbon atoms, which is attached to the rest of the molecule by a single bond. Non-limiting examples of “C₂-C₃alkenyl” groups include ethenyl (a C₂alkenyl) and prop-1-enyl (a C₃alkenyl).

The term “alkylene”, as used herein, refers to a bivalent straight or branched hydrocarbon chain radical consisting solely of carbon and hydrogen atoms and containing no unsaturation. The term “C₁-C₆alkylene”, as used herein, refers to a bivalent straight or branched hydrocarbon chain radical consisting solely of carbon and hydrogen atoms, containing no unsaturation, having from one to six carbon atoms. Non-limiting examples of “C₁-C₆alkylene” groups include methylene (a C₁alkylene), ethylene (a C₂alkylene), 1-methylethylene (a C₃alkylene), n-propylene (a C₃alkylene), isopropylene (a C₃alkylene), n-butylene (a C₄alkylene), isobutylene (a C₄alkylene), sec-butylene (a C₄alkylene), tert-butylene (a C₄alkylene), n-pentylene (a C₅alkylene), isopentylene (a C₅alkylene), neopentylene (a C₅alkylene), and hexylene (a C₆alkylene).

The term “alkenylene”, as used herein, refers to a bivalent straight or branched hydrocarbon chain radical consisting solely of carbon and hydrogen atoms and containing at least one double bond. The term “C₂-C₆alkenylene”, as used herein, refers to a bivalent straight or branched hydrocarbon chain radical group consisting solely of carbon and hydrogen atoms, containing at least one double bond, and having from two to six carbon atoms. Non-limiting examples of “C₂-C₆alkenylene” groups include ethenylene (a C₂alkenylene), prop-1-enylene (a C₃alkenylene), but-1-enylene (a C₄alkenylene), pent-1-enylene (a C₅alkenylene), pent-4-enylene (a C₅alkenylene), penta-1,4-dienylene (a C₅alkenylene), hexa-1-enylene (a C₆alkenylene), hexa-2-enylene (a C₆alkenylene), hexa-3-enylene (a C₆alkenylene), hexa-1-,4-dienylene (a C₆alkenylene), hexa-1-,5-dienylene (a C₆alkenylene) and hexa-2-,4-dienylene (a C₆alkenylene). The term “C₂-C₆alkenylene”, as used herein, refers to a bivalent straight or branched hydrocarbon chain radical group consisting solely of carbon and hydrogen atoms, containing at least one double bond, and having from two to the carbon atoms. Non-limiting examples of “C₂-C₃alkenylene” groups include ethenylene (a C₂alkenylene) and prop-1-enylene (a C₃alkenylene).

The term “cycloalkyl,” or “C₃-C₈cycloalkyl,” as used herein, refers to a saturated, monocyclic, fused bicyclic, fused tricyclic or bridged polycyclic ring system. Non-limiting examples of fused bicyclic or bridged polycyclic ring systems include bicyclo[1.1.1]pentane, bicyclo[2.1.1]hexane, bicyclo[2.2.1]heptane, bicyclo[3.1.1]heptane, bicyclo[3.2.1]octane, bicyclo[2.2.2]octane and adamantanyl. Non-limiting examples monocyclic C₃-C₈cycloalkyl groups include cyclopropyl, cyclobutyl, cyclopentyl, cyclohexyl, cycloheptyl and cyclooctyl groups.

The term “polyethylene glycol” or “PEG”, as used herein, refers to a linear chain, a branched chain or a star shaped configuration comprised of (OCH₂CH₂) groups. In certain embodiments a polyethylene or PEG group is —(OCH₂CH₂)ₜ*—, where t is 4-40, and where the “—” indicates the end directed toward the self-immolative spacer and the “*—” indicates the point of attachment to a terminal end group R′ where R′ is OH, OCH₃ or OCH₂CH₂C(═O)OH. In other embodiments a polyethylene or PEG group is —(CH₂CH₂O)ₜ*—, where t is 4-40, and where the “—” indicates the end directed toward the self-immolative spacer and the “*—” indicates the point of attachment to a terminal end group R″ where R″ is H, CH₃ or CH₂CH₂C(═O)OH.

The term “polyalkylene glycol”, as used herein, refers to a linear chain, a branched chain or a star shaped configuration comprised of (O(CH₂)ₘ)ₜ groups. In certain embodiments a polyethylene or PEG group is —(O(CH₂)ₘ)ₜ*—, where m is 1-10, t is 4-40, and where the “—” indicates the end directed toward the self-immolative spacer and the “*—” indicates the point of attachment to a terminal end group R′ where R′ is OH, OCH₃ or OCH₂CH₂C(═O)OH. In other embodiments a polyethylene or PEG group is —((CH₂)ₘO)ₜ*—, where m is 1-10, t is 4-40, and where the “—” indicates the end directed toward the self-immolative spacer and the “*—” indicates the point of attachment to a terminal end group R″ where R″ is H, CH₃ or CH₂CH₂C(═O)OH.

The terms “Drug moiety”, “D”, or “drug”, as used herein, refer to any compound possessing a desired biological activity and a reactive functional group that may be used to incorporate the drug into the linker-drug group of the invention. The desired biological activity includes the diagnosis, cure, mitigation, treatment, or prevention of disease in man or other animals. The reactive functional group forms a bond to the “A” in compounds of Formula (I) and Formula (II) and conjugates of Formula (III) and Formula (IV). In some embodiments, the Drug moiety has a nitrogen atom that can form a bond with “A”. In other embodiments, the Drug moiety has a hydroxyl group that can form a bond with “A”. In other embodiments, the Drug moiety has a carboxylic acid that can form a bond with “A”. In other embodiments, the Drug moiety has a carbonyl group that can form a bond with “A”. In still other embodiments, the Drug moiety has a sulfhydryl group that can form a bond with “A”.

Provided that the needed reactive functional group is present, the terms “drug moiety”, “D” or “drug” further refer to chemicals recognized as drugs in the official United States Pharmacopeia, official Homeopathic Pharmacopeia of the United States, or official National Formulary, or any supplement thereof Exemplary drugs are set forth in the Physician's Desk Reference (PDR) and in the Orange Book maintained by the U.S. Food and Drug Administration (FDA).

In one embodiment, the Drug moiety (D) can be a cytotoxic, cytostatic or immunosuppressive drug. Such cytotoxic or immunosuppressive drugs include, for example, antitubulin agents, tubulin inhibitors, DNA minor groove binders, DNA replication inhibitors, alkylating agents, antibiotics, antifolates, antimetabolites, chemotherapy sensitizers, topoisomerase inhibitors, vinca alkaloids, or the like. Examples of such cytotoxic drugs include, for example, auristatins, camptothecins, duocarmycins, etoposides, maytansines and maytansinoids, taxanes, benzodiazepines or benzodiazepine containing drugs (e.g., pyrrolo[1,4]-benzodiazepines (PBDs), indolinobenzodiazepines, and oxazolidinobenzodiazepines) and vinca alkaloids.

The effects of the present invention will be more pronounced in embodiments wherein the Drug moiety is hydrophobic. Accordingly, the Drug moiety of the present invention is preferably hydrophobic having a S log P value of 1.5 or greater, 2.0 or greater, or 2.5 or greater. In some aspects, drugs to be used in the present invention will have a S log P value from (a) about 1.5, about 2, or 2.5 to about 7, (b) about 1.5, about 2, or 2.5 to about 6, (c) about 1.5, about 2 or about 2.5 to about 5, (d) about 1.5, about 2, or 2.5 to about 4, or (e) about 1.5, about 2 or about 2.5 to about 3.

Hydrophobicity can be measured using S log P. S log P is defined as the log of the octanol/water partition coefficient (including implicit hydrogens) and can be calculated using the program MOE™ from the Chemical Computing group (S log P values calculated using Wildman, S. A., Crippen, G. M.; Prediction of Physiochemical Parameters by Atomic Contributions; J. Chern. Inf Comput. Sci. 39 No. 5 (1999) 868-873).

The term “reactive group”, as used herein, is a functional group capable of forming a covalent bond with a functional group of an antibody or antibody fragment. Non limiting examples of such functional groups include reactive groups of Table 1 provided herein.

The term “coupling group”, as used herein, refers to a bivalent moiety which links the bridging spacer to the antibody or fragment thereof. The coupling group is a bivalent moiety formed by the reaction between a reaction group and a functional group on the antibody or fragment thereof. Non limiting examples of such bivalent moieties include the bivalent chemical moieties given in Table 1 and Table 2 provided herein.

The term “bridging spacer”, as used herein, refers to one or more linker components which are covalently attached together to form a bivalent moiety which links the bivalent peptide spacer to the reactive group or links the bivalent peptide space to the coupling group. In certain embodiments the “bridging spacer” comprises a carboxyl group attached to the N-terminus of the bivalent peptide spacer via an amide bond.

The term “spacer moiety”, as used herein, refers to one or more linker components which are covalently attached together to form a moiety which links the self-immolative spacer to the hydrophilic moiety.

The term “bivalent peptide spacer”, as used herein, refers to bivalent linker comprising one or more amino acid residues covalently attached together to form a moiety which links the bridging spacer to the self immolative spacer. The one or more amino acid residues can be an residue of amino acids selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine.

In certain embodiments a “bivalent peptide spacer” is a combination of 2 to four amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example -ValCit*; -CitVal*; -AlaAla*; -AlaCit*; -CitAla*; -AsnCit*; -CitAsn*; -CitCit*; -ValGlu*; -GluVal*; -SerCit*; -CitSer*; -LysCit*; -CitLys*; -AspCit*; -CitAsp*; -AlaVal*; -ValAla*; -PheAla*; -AlaPhe*; -PheLys*; -LysPhe*; -ValLys*; -LysVal*; -AlaLys*; -LysAla*; -PheCit*; -CitPhe*; -LeuCit*; -CitLeu*; -IleCit*; -Citlle*; -PheArg*; -ArgPhe*; -CitTrp*; -TrpCit*; -PhePheLys*; -LysPhePhe*; -DPhePheLys*; -DLysPhePhe*; -GlyPheLys*; -LysPheGly*; -GlyPheLeuGly- [SEQ ID NO:160]; -GlyLeuPheGly- [SEQ ID NO:161]; -AlaLeuAlaLeu-[SEQ ID NO:162], -GlyGlyGly*; -GlyGlyGlyGly- [SEQ ID NO:163]; -GlyPheValGly-[SEQ ID NO:164]; and -GlyValPheGly- [SEQ ID NO:165], where the “—” indicates the point of attachment to the bridging spacer and the “*” indicates the point of attachment to the self-immolative spacer.

The term “linker component”, as used herein, refers to the following
a) an alkylene group: —(CH₂)ₙ— which can either be linear or branched (where in this instance n is 1-18);
b) an alkenylene group;
c) an alkynylene group;
d) an alkenyl group;
e) an alkynyl group;
f) an ethylene glycol unit: —OCH₂CH₂ or —CH₂CH₂O;
g) an polyethylene glycol unit: (—CH₂CH₂O—)ₓ (where x in this instance is 2-20);
h) —O;
i) —S;
j) a carbonyl: —C(═O);
k) an ester: —C(═O)—O— or —O—C(═O);
l) a carbonate: —OC(═O)O;
m) an amine: —NH;
n) an tertiary amine
o) an amides: —C(═O)—NH—, —NH—C(═O)— or —C(═O)N(C₁₋₆alkyl);
p) a carbamate: —OC(═O)NH— or —NHC(═O)O;
q) a urea: —NHC(═O)NH;
r) a sulphonamide: —S(O)₂NH— or —NHS(O)₂;
s) an ether: —CH₂O— or —OCH₂;
t) an alkylene substituted with one or more groups independently selected from carboxy, sulfonate, hydroxyl, amine, amino acid, saccharide, phosphate and phosphonate);
u) an alkenylene substituted with one or more groups independently selected from carboxy, sulfonate, hydroxyl, amine, amino acid, saccharide, phosphate and phosphonate);
v) an alkynylene substituted with one or more groups independently selected from carboxy, sulfonate, hydroxyl, amine, amino acid, saccharide, phosphate and phosphonate);
w) a C₁-C₁₀alkylene in which one or more methylene groups is replace by one or more —S—, —NH— or —O— moieties;
x) a ring systems having two available points of attachment such as a divalent ring selected from phenyl (including 1,2-1,3- and 1,4- di-substituted phenyls), a C₅-C₆ heteroaryl, a C₃-C₈cycloalkyl (including 1,1-disubstituted cyclopropyl, cyclobutyl, cyclopentyl or cyclohexyl, and 1,4-disubstituted cyclohexyl), and a C₄-C₈ heterocycloalkyl;
y) a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine;
   a combination of 2 or more amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit; and
z) a self-immolative spacer, wherein the self-immolative spacer comprises one or more protecting (triggering) groups which are susceptible to acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage

Non-limiting examples of such self-immolative spacer include:

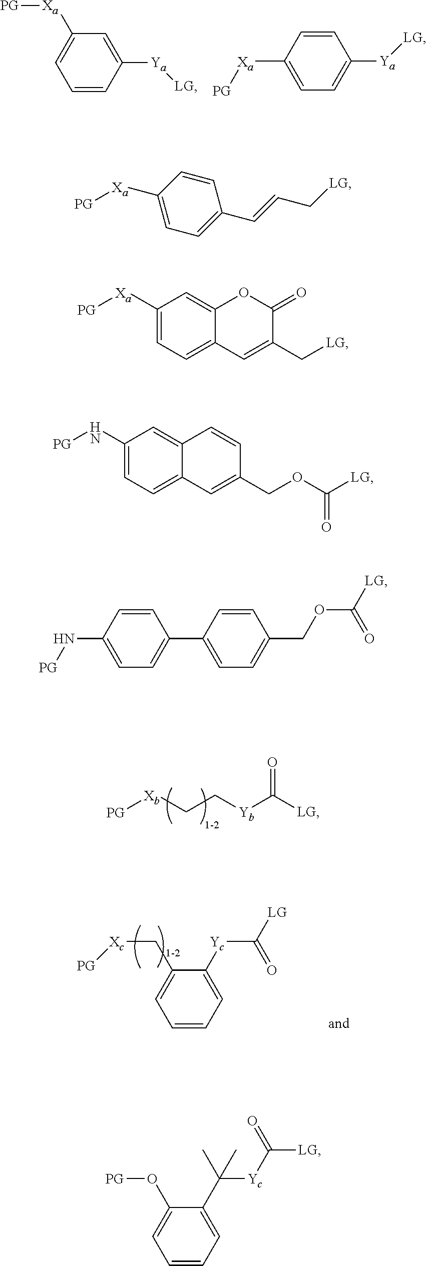

where:
PG is a protecting (triggering) group;
Xₐ is O, NH or S;
X_{b} is O, NH, NCH₃ or S;
X_{c} is O or NH;
Yₐ is CH₂, CH₂O or CH₂NH;
Y_{b} is CH₂, O or NH;
Y_{c} is a bond, CH₂, O or NH, and
LG is a leaving group such as a Drug moiety (D) of the Linker-Drug group of the invention.
Additional non-limiting examples of such self-immolative spacers are described in Angew. Chem. Int. Ed. 2015, 54, 7492-7509.

In addition, a linker component can be a chemical moiety which is readily formed by reaction between two reactive groups. Non-limiting examples of such chemical moieties are given in Table 1.

TABLE 1

| Reactive Group 1 (RG1) | Reactive Group 2 (RG2) | Chemical Moiety |
|---|---|---|
| a thiol | a thiol | —S—S— |
| a thiol | a maleimide | 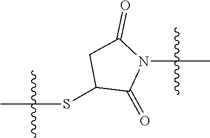 [SMILES: *SC1CC(=O)N(*)C1=O] |
| a thiol | a haloacetamide | 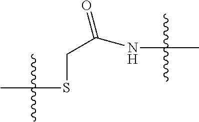 [SMILES: *NC(=O)CS*] |
| an azide | an alkyne | 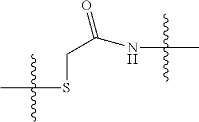 [SMILES: *NC(=O)CS*] |
| an azide | a triaryl phosphine | 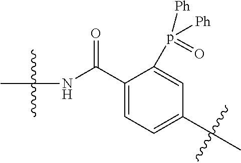 |
| an azide | a cyclooctyne | 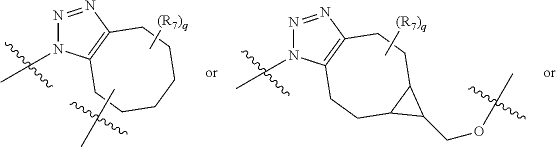 |
| | | 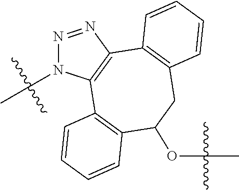 [SMILES: *OC1Cc2ccccc2-c2nnn(*)c2-c2ccccc21] |
| an azide | an oxanobornadiene | 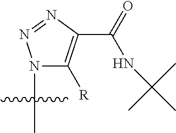 |
| a triaryl phosphine | an azide | 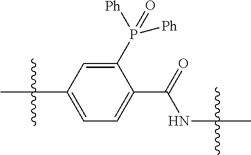 [SMILES: *NC(=O)c1ccc(*)cc1P(=O)(c1ccccc1)c1ccccc1] |
| an oxanobornadiene | an azide | 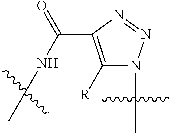 |
| an alkyne | an azide | 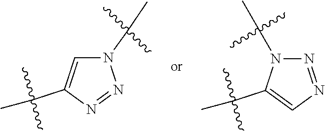 [SMILES: *c1cn(*)nn1 ; *c1cnnn1*] |
| a cyclooctyne | azide | 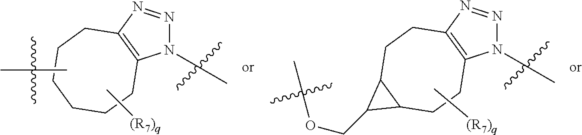 |
| | | 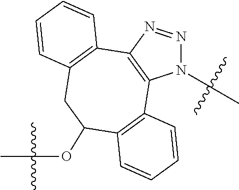 [SMILES: *OC1Cc2ccccc2-c2nnn(*)c2-c2ccccc21] |
| a cyclooctene | a diaryl tetrazine | 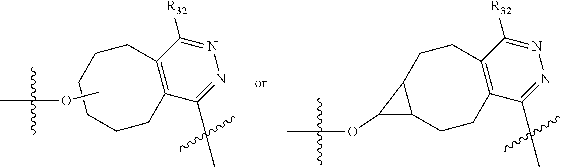 |
| a diaryl tetrazine | a cyclooctene | 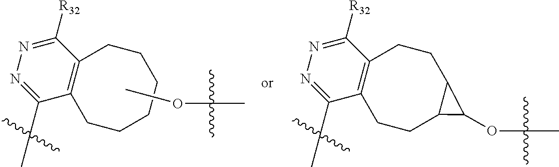 |
| a monoaryl tetrazine | a norbornene | 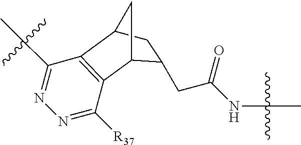 |
| a norbornene | a monoaryl tetrazine | 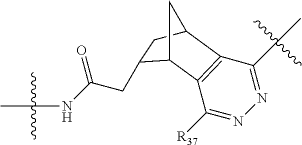 |
| an aldehyde | a hydroxylamine | 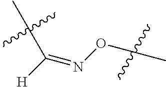 [SMILES: */C=N\O*] |
| an aldehyde | a hydrazine | 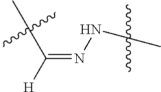 [SMILES: */C=N\N*] |
| an aldehyde | NH₂—NH—C(═O)— | 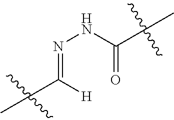 [SMILES: */C=N/NC(*)=O] |
| a ketone | a hydroxylamine | 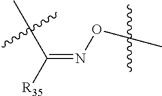 |
| a ketone | a hydrazine | 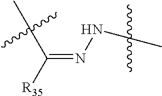 |
| a ketone | NH₂—NH—C(═O)— | 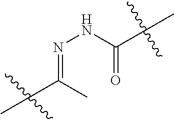 [SMILES: *C(=O)N/N=C(/*)C] |
| a hydroxylamine | an aldehyde | 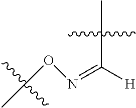 [SMILES: */C=N\O*] |
| a hydroxylamine | a ketone | 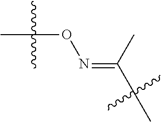 [SMILES: *O/N=C(/*)C] |
| a hydrazine | an aldehyde | 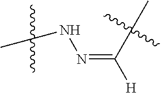 [SMILES: */C=N\N*] |
| a hydrazine | a ketone | 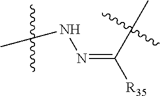 |
| NH₂—NH—C(═O)— | an aldehyde | 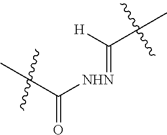 |
| NH₂—NH—C(═O)— | a ketone | 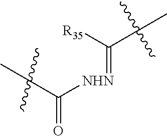 |
| a haloacetamide | a thiol | 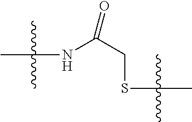 [SMILES: *NC(=O)CS*] |
| a maleimide | a thiol | 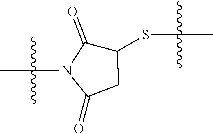 [SMILES: *SC1CC(=O)N(*)C1=O] |
| a vinyl sulfone | a thiol | 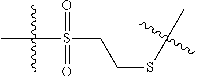 [SMILES: *SCCS(*)(=O)=O] |
| a thiol | a vinyl sulfone | 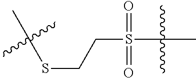 [SMILES: *SCCS(*)(=O)=O] |
| an aziridine | a thiol | 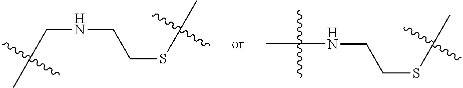 [SMILES: *CNCCS* ; *NCCS*] |
| a thiol | an aziridine | 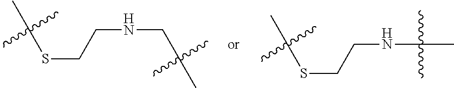 [SMILES: *CNCCS* ; *NCCS*] |
| 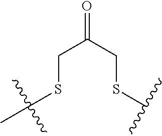 [SMILES: *SCC(=O)CS*] | hydroxylamine | 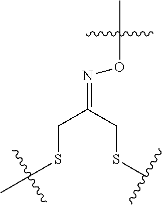 [SMILES: *ON=C(CS*)CS*] |
| 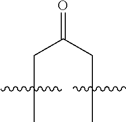 [SMILES: *CC(=O)C*] | hydroxylamine | 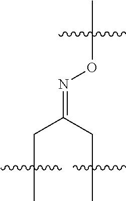 [SMILES: *CC(C*)=NO*] |
| 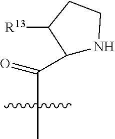 | 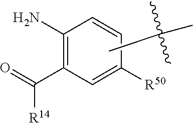 | 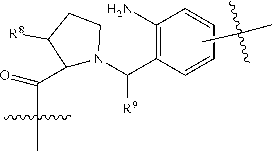 |
| 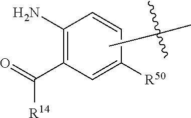 | 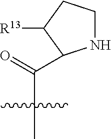 | 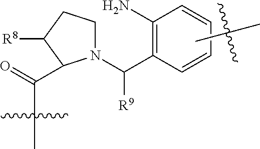 |
| 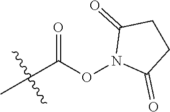 [SMILES: *C(=O)ON1C(=O)CCC1=O] | —NH₂, | amide |
| 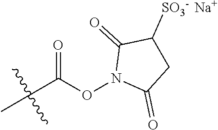 | | |
| 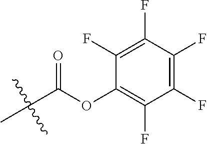 [SMILES: *C(=O)Oc1c(F)c(F)c(F)c(F)c1F] | | |
| 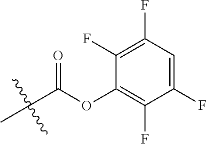 [SMILES: *C(=O)Oc1c(F)c(F)cc(F)c1F] | | |
| 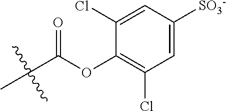 | | |
| —NH₂, | 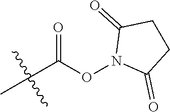 [SMILES: *C(=O)ON1C(=O)CCC1=O] | amide |
| | 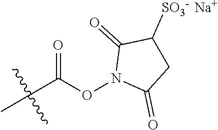 | |
| | 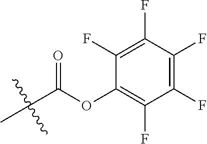 [SMILES: *C(=O)Oc1c(F)c(F)c(F)c(F)c1F] | |
| | 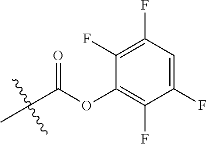 [SMILES: *C(=O)Oc1c(F)c(F)cc(F)c1F] | |
| | 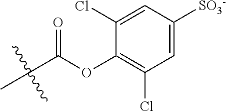 | |
| CoA or CoA analogue | Serine residue | 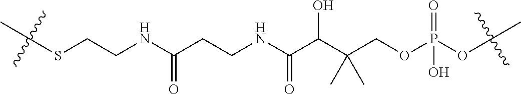 [SMILES: *OP(=O)(O)OCC(C)(C)C(O)C(=O)NCCC(=O)NCCS*] |
| | | 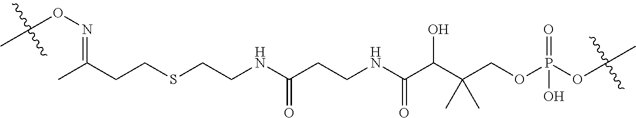 [SMILES: *O/N=C(\C)CCSCCNC(=O)CCNC(=O)C(O)C(C)(C)COP(=O)(O)O*] |
| | | 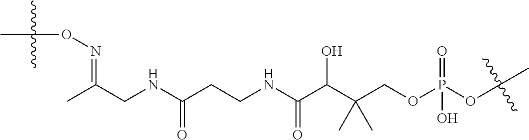 [SMILES: *O/N=C(\C)CNC(=O)CCNC(=O)C(O)C(C)(C)COP(=O)(O)O*] |
| | | 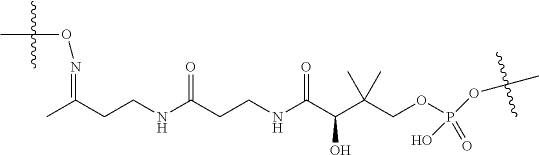 [SMILES: *O/N=C(\C)CCNC(=O)CCNC(=O)[C@H](O)C(C)(C)COP(=O)(O)O*] |
| | | 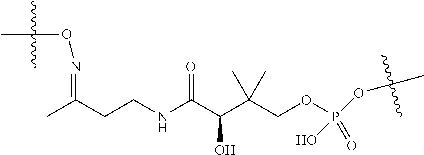 [SMILES: *O/N=C(\C)CCNC(=O)[C@H](O)C(C)(C)COP(=O)(O)O*] |
| | | 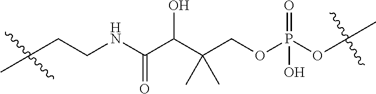 [SMILES: *CCNC(=O)C(O)C(C)(C)COP(=O)(O)O*] |
| | | 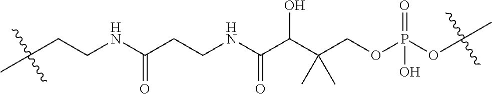 [SMILES: *CCNC(=O)CCNC(=O)C(O)C(C)(C)COP(=O)(O)O*] |
| | | 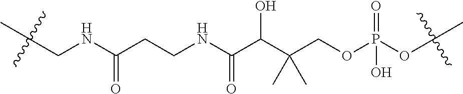 [SMILES: *CNC(=O)CCNC(=O)C(O)C(C)(C)COP(=O)(O)O*] |
| pyridyldithiol | thiol | disulfide |

where: R³² in Table 1 is H, C₁₋₄ alkyl, phenyl, pyrimidine or pyridine; R³⁵ in Table 1 is H, C₁₋₆alkyl, phenyl or C₁₋₄alkyl substituted with 1 to 3 —OH groups; each R⁷ in Table 1 is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH; R³⁷ in Table 1 is independently selected from H, phenyl and pyridine; q in Table 1 is 0, 1, 2 or 3; R⁸ or R¹³ in Table 1 is H or methyl; and R⁹ or R¹⁴ in Table 1 is H, —OH₃ or phenyl; and R in Table 1 is H or a suitable substituent, e.g., alkyl.

In addition, a linker component can be a group given in Table 2 below.

TABLE 2

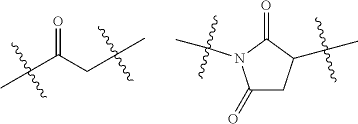 [SMILES: *CC(*)=O ; *C1CC(=O)N(*)C1=O]

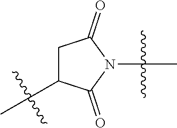 [SMILES: *C1CC(=O)N(*)C1=O]

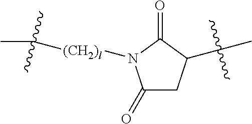

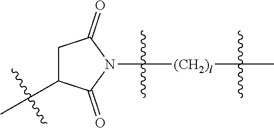

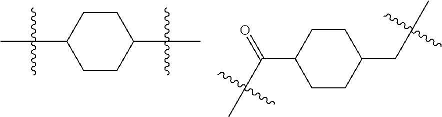 [SMILES: *C1CCC(*)CC1 ; *CC1CCC(C(*)=O)CC1]

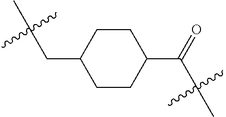 [SMILES: *CC1CCC(C(*)=O)CC1]

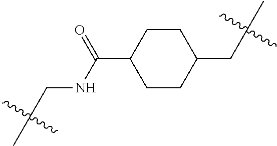 [SMILES: *CNC(=O)C1CCC(C*)CC1]

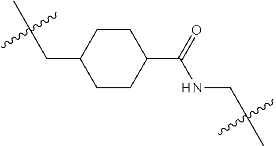 [SMILES: *CNC(=O)C1CCC(C*)CC1]

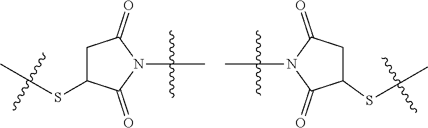 [SMILES: *SC1CC(=O)N(*)C1=O ; *SC1CC(=O)N(*)C1=O]

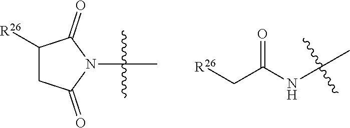

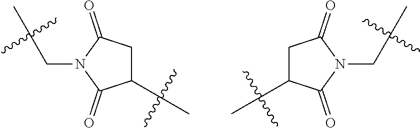 [SMILES: *CN1C(=O)CC(*)C1=O ; *CN1C(=O)CC(*)C1=O]

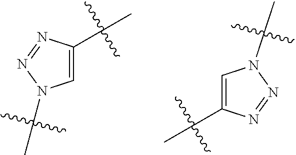 [SMILES: *c1cn(*)nn1 ; *c1cn(*)nn1]

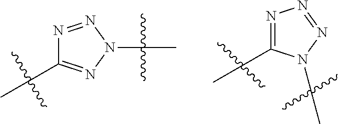 [SMILES: *c1nnn(*)n1 ; *c1nnnn1*]

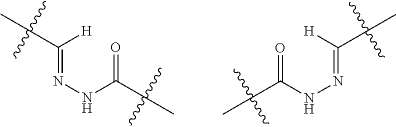 [SMILES: */C=N/NC(*)=O ; */C=N/NC(*)=O]

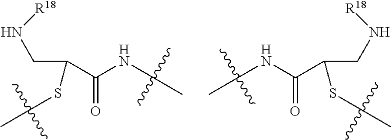

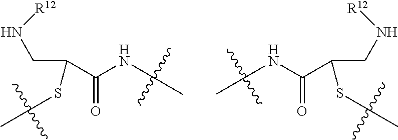

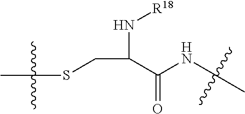

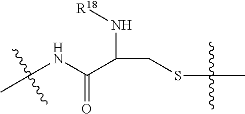

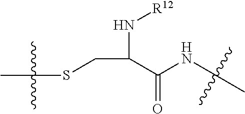

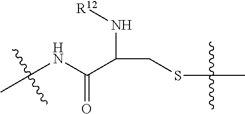

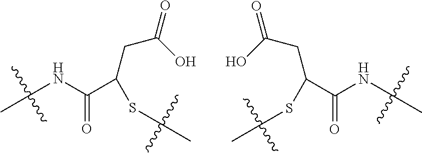 [SMILES: *NC(=O)C(CC(=O)O)S* ; *NC(=O)C(CC(=O)O)S*]

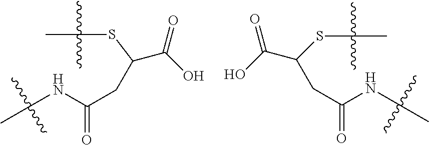 [SMILES: *NC(=O)CC(S*)C(=O)O ; *NC(=O)CC(S*)C(=O)O]

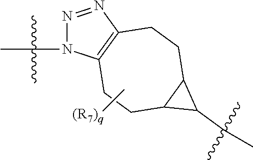

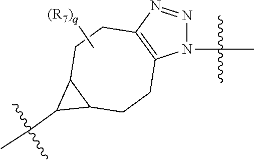

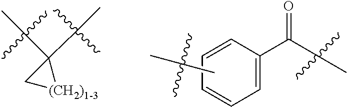

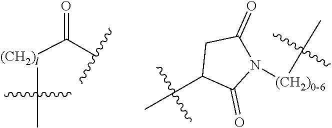

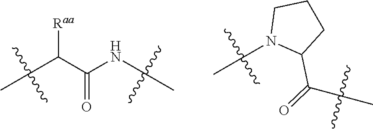

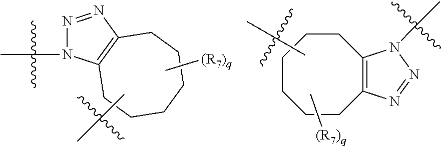

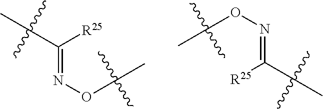

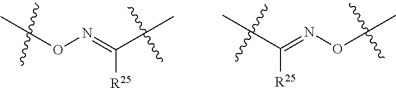

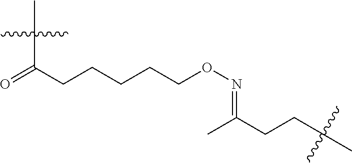 [SMILES: *CC/C(C)=N/OCCCCCC(*)=O]

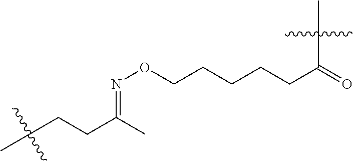 [SMILES: *CC/C(C)=N/OCCCCCC(*)=O]

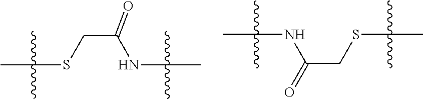 [SMILES: *NC(=O)CS* ; *NC(=O)CS*]

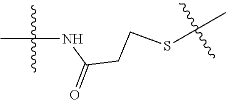 [SMILES: *NC(=O)CCS*]

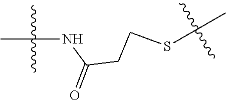 [SMILES: *NC(=O)CCS*]

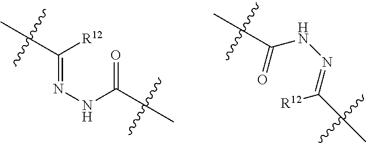

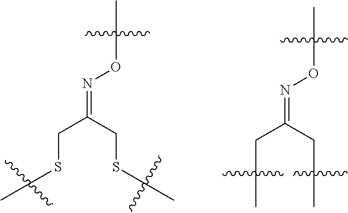 [SMILES: *ON=C(CS*)CS* ; *CC(C*)=NO*]

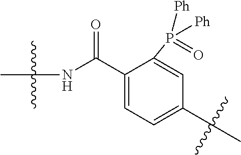 [SMILES: *NC(=O)c1ccc(*)cc1P(=O)(c1ccccc1)c1ccccc1]

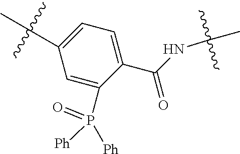 [SMILES: *NC(=O)c1ccc(*)cc1P(=O)(c1ccccc1)c1ccccc1]

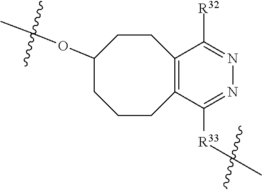

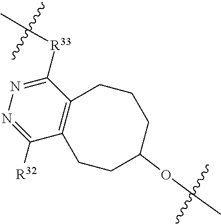

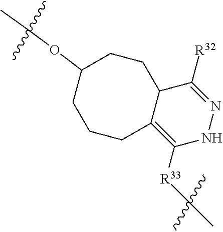

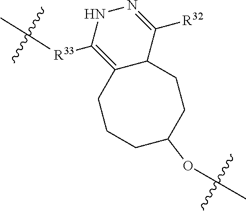

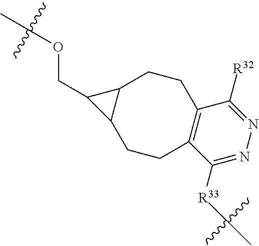

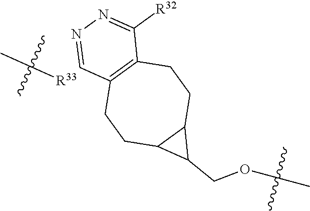

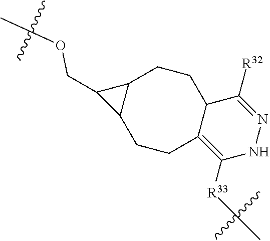

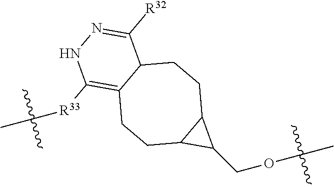

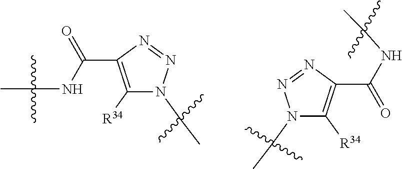

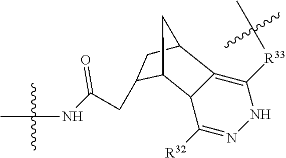

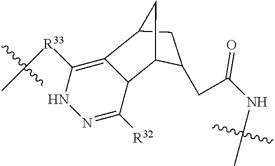

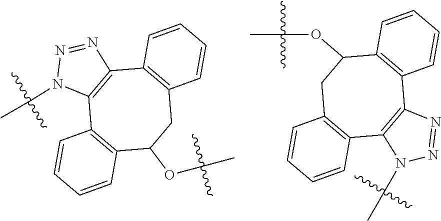 [SMILES: *OC1Cc2ccccc2-c2nnn(*)c2-c2ccccc21 ; *OC1Cc2ccccc2-c2c(nnn2*)-c2ccccc21]

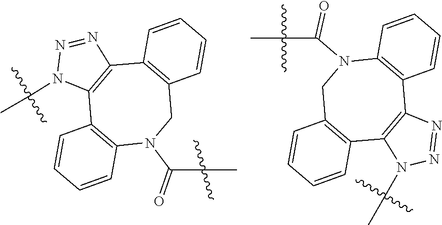 [SMILES: *C(=O)N1Cc2ccccc2-c2nnn(*)c2-c2ccccc21 ; *C(=O)N1Cc2ccccc2-c2c(nnn2*)-c2ccccc21]

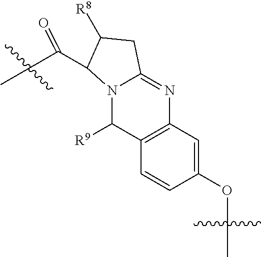

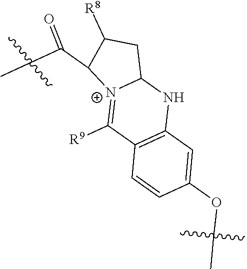

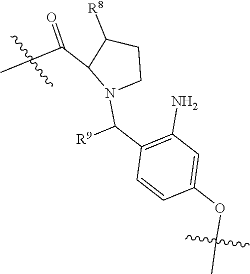

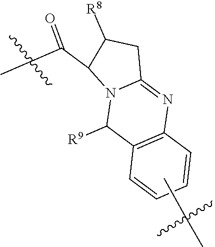

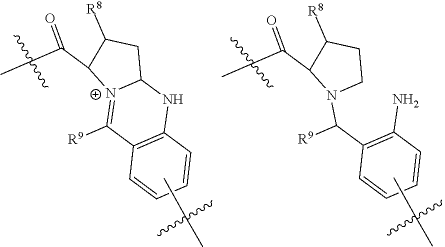

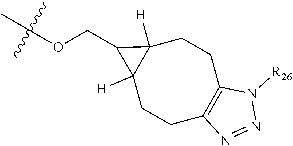

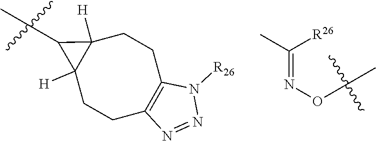

each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each R¹² is independently selected from H and C₁-C₆alkyl
R⁸ is H or methyl;
R⁹ is H, —CH₃ or phenyl;
each R²⁸ is independently selected from H or C₁₋₄ alkyl;
each R¹⁸ is independently selected from a C₁-C₆alkyl, a C₁-C₆alkyl which is substituted with azido and a C₁₋C₆alkyl which is substituted with 1 to 5 hydroxyl;
q is 0, 1, 2 or 3;
l is 1, 2, 3, 4, 5 or 6; 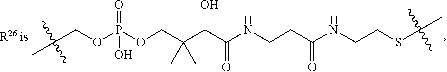 [SMILES: *COP(=O)(O)OCC(C)(C)C(O)C(=O)NCCC(=O)NCCS*] 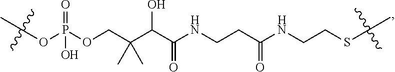 [SMILES: *OP(=O)(O)OCC(C)(C)C(O)C(=O)NCCC(=O)NCCS*] 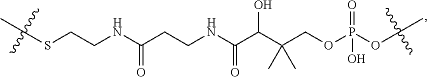 [SMILES: *OP(=O)(O)OCC(C)(C)C(O)C(=O)NCCC(=O)NCCS*] 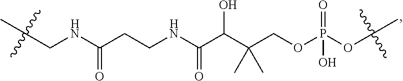 [SMILES: *CNC(=O)CCNC(=O)C(O)C(C)(C)COP(=O)(O)O*] 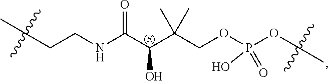 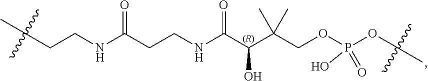 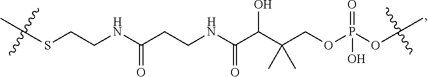 [SMILES: *OP(=O)(O)OCC(C)(C)C(O)C(=O)NCCC(=O)NCCS*] 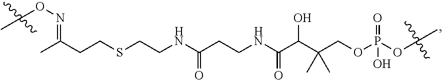 [SMILES: *O/N=C(\C)CCSCCNC(=O)CCNC(=O)C(O)C(C)(C)COP(=O)(O)O*] 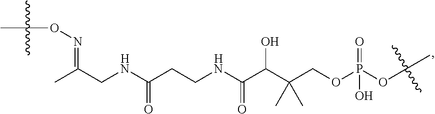 [SMILES: *O/N=C(\C)CNC(=O)CCNC(=O)C(O)C(C)(C)COP(=O)(O)O*] 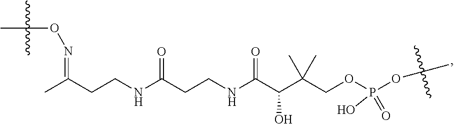 [SMILES: *O/N=C(\C)CCNC(=O)CCNC(=O)[C@@H](O)C(C)(C)COP(=O)(O)O*] 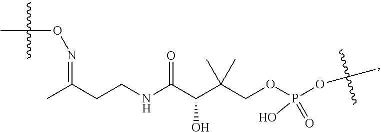 [SMILES: *O/N=C(\C)CCNC(=O)[C@@H](O)C(C)(C)COP(=O)(O)O*] 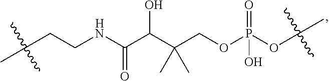 [SMILES: *CCNC(=O)C(O)C(C)(C)COP(=O)(O)O*] 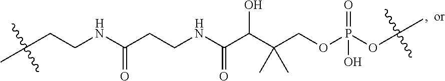 [SMILES: *CCNC(=O)CCNC(=O)C(O)C(C)(C)COP(=O)(O)O*] 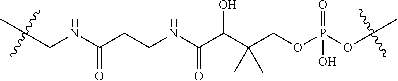 [SMILES: *CNC(=O)CCNC(=O)C(O)C(C)(C)COP(=O)(O)O*]
R³² is independently selected from H, C₁₋₄ alkyl, phenyl, pyrimidine and pyridine; 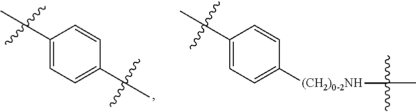
R³³ is independently selected from 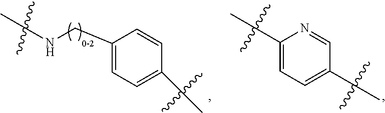 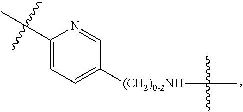 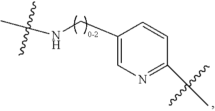 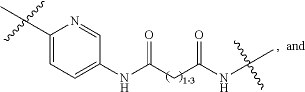 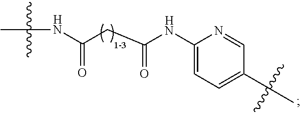
R³⁴ is independently selected from H, C₁₋₄ alkyl, and C₁₋₆ haloalkyl, and
R^{aa} is an amino acid side chain.

As used herein, when partial structures of the compounds are illustrated a wavy line ( [symbol] ) indicates the point of attachment of the partial structure to the rest of the molecule.

The term “self-immolative spacer”, as used herein, refers a moiety comprising one or more triggering groups (TG) which are activated by acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage, and after activation the protecting group is removed, which generates a cascade of disassembling reactions leading to the temporally sequential release of a leaving group. Such cascade of reactions can be, but not limited to, 1,4-, 1,6- or 1,8-elimination reactions.

Non-limiting examples of such self-immolative spacer include:

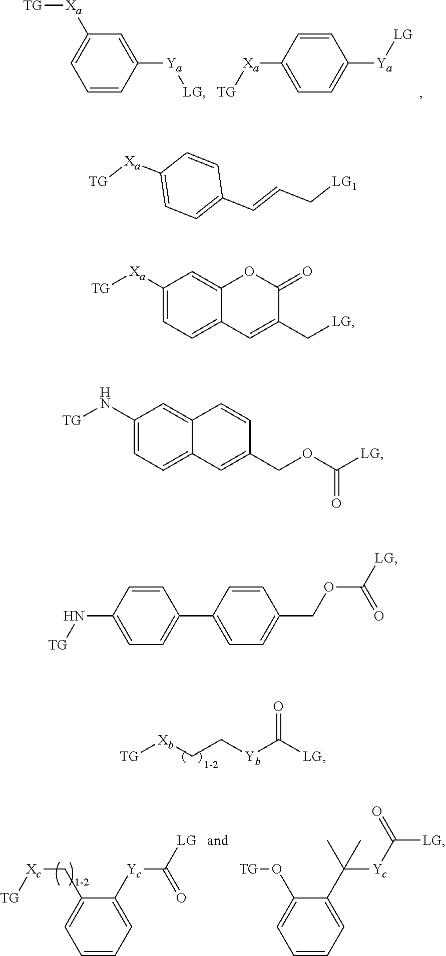

wherein such groups can be optionally substituted, and

where:
TG is a triggering group;
Xₐ is O, NH or S;
X_{b} is O, NH, NCH₃ or S;
X_{c} is O or NH;
Yₐ is CH₂, CH₂O or CH₂NH;
Y_{b} is CH₂, O or NH;
Y_{c} is a bond, CH₂, O or NH, and
LG is a leaving group such as a Drug moiety (D) of the Linker-Drug group of the invention.
Additional non-limiting examples of such self-immolative spacers are described in Angew. Chem. Int. Ed. 2015, 54, 7492-7509.

In certain embodiment the self-immolative spacer is moiety having the structure

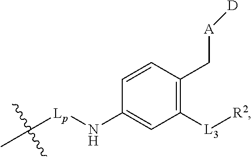

Where Lp is an enzymatically cleavable bivalent peptide spacer and A, D, L₃ and R² are as defined herein.

In preferred embodiments, the self-immolative spacer is moiety having the structure

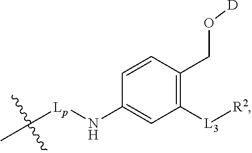

where Lp is an enzymatically cleavable bivalent peptide spacer and D, L₃ and R² are as defined herein.

In other preferred embodiments, the self-immolative spacer is moiety having the structure

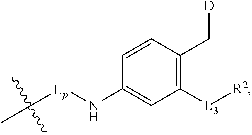

where Lp is an enzymatically cleavable bivalent peptide spacer and D, L₃ and R² are as defined herein. In some embodiments, D is a quaternized tertiary amine-containing Drug moiety, wherein the ammonium cation optionally exists as a zwitterionic form or has a monovalent anionic counterion.

The term “hydrophilic moiety”, as used herein, refers to moiety that is has hydrophilic properties which increases the aqueous solubility of the Drug moiety (D) when the Drug moiety (D) is attached to the linker group of the invention. Examples of such hydrophilic groups include, but are not limited to, polyethylene glycols, polyalkylene glycols, sugars, oligosaccharides, polypeptides a C₂-C₆alkyl substituted with 1 to 3

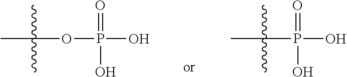 [SMILES: *OP(=O)(O)O ; *P(=O)(O)O]

groups and polysarcosines such as those having the formula of

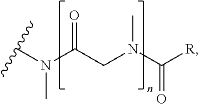

wherein n is an integer between 2 and 25; and R is H, —CH₃ or —CH₂CH₂C(═O)OH.

In some embodiments, the hydrophilic moiety comprises a polyethylene glycol of formula:

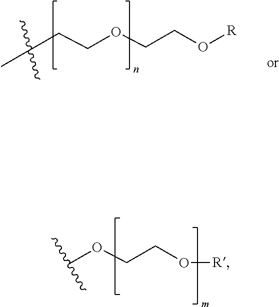

wherein R is H, —CH₃, —CH₂CH₂NHC(═O)ORₐ, —CH₂CH₂NHC(═O)Rₐ, or —CH₂CH₂C(═O)ORₐ, R′ is OH, —OCH₃, —CH₂CH₂NHC(═O)ORₐ, —CH₂CH₂NHC(═O)Rₐ, or —OCH₂CH₂C(═O)ORₐ in which Rₐ is H or C₁₋₄ alkyl optionally substituted with either OH or C₁₋₄ alkoxyl, and each of m and n is an integer between 2 and 25 (e.g., between 3 and 25). In some embodiments, the hydrophilic moiety comprises

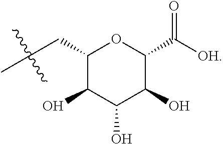 [SMILES: *C[C@@H]1O[C@H](C(=O)O)[C@@H](O)[C@H](O)[C@H]1O]

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2) or subclass.

The term “antibody fragment” or “antigen-binding fragment” or “functional fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies). The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof, and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). In a combined Kabat and Chothia numbering scheme for a given CDR region (for example, HC CDR1, HC CDR2, HC CDR3, LC CDR1, LC CDR2 or LC CDR3), in some embodiments, the CDRs correspond to the amino acid residues that are defined as part of the Kabat CDR, together with the amino acid residues that are defined as part of the Chothia CDR. As used herein, the CDRs defined according to the “Chothia” number scheme are also sometimes referred to as “hypervariable loops.”

For example, under Kabat, the CDR amino acid residues in the heavy chain variable domain (VH) are numbered 31-35 (HCDR1) (e.g., insertion(s) after position 35), 50-65 (HCDR2), and 95-102 (HCDR3); and the CDR amino acid residues in the light chain variable domain (VL) are numbered 24-34 (LCDR1) (e.g., insertion(s) after position 27), 50-56 (LCDR2), and 89-97 (LCDR3). As another example, under Chothia, the CDR amino acids in the VH are numbered 26-32 (HCDR1) (e.g., insertion(s) after position 31), 52-56 (HCDR2), and 95-102 (HCDR3); and the amino acid residues in VL are numbered 26-32 (LCDR1) (e.g., insertion(s) after position 30), 50-52 (LCDR2), and 91-96 (LCDR3). By combining the CDR definitions of both Kabat and Chothia, the CDRs comprise or consist of, e.g., amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT, the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align.

The term “epitope” includes any protein determinant capable of specific binding to an immunoglobulin or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational.” Conformational and linear epitopes are distinguished in that the binding to the former but not the latter is lost in the presence of denaturing solvents.

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

The phrase “human antibody,” as used herein, includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia, and ImMunoGenTics (IMGT) numbering (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5th edit, NIH Publication no. 91-3242 U.S. Department of Health and Human Services; Chothia et al., (1987) J. Mol. Biol. 196:901-917; Chothia et al., (1989) Nature 342:877-883; AI-Lazikani et al., (1997) J. Mal. Biol. 273:927-948; and Lefranc, M.-P., The Immunologist, 7, 132-136 (1999); Lefranc, M.-P. et al., Dev. Comp. Immunol., 27, 55-77 (2003)).

The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant human antibody” as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

The term “binding specificity” as used herein refers to the ability of an individual antibody combining site to react with one antigenic determinant and not with a different antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within an antibody can be replaced with other amino acid residues from the same side chain family and the altered antibody can be tested using the functional assays described herein.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See www.ncbi.nlm.nih.gov.

The terms “composition” or “pharmaceutical composition,” as used herein, refers to a mixture of a compound of the invention with at least one and optionally more than one other pharmaceutically acceptable chemical components, such as carriers, stabilizers, diluents, dispersing agents, suspending agents, thickening agents, and/or excipients.

The term “an optical isomer” or “a stereoisomer”, as used herein, refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non- superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold- Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

The term “pharmaceutically acceptable carrier”, as used herein, includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “pharmaceutically acceptable salt,” as used herein, refers to a salt which does not abrogate the biological activity and properties of the compounds of the invention, and does not cause significant irritation to a subject to which it is administered.

The term “subject”, as used herein, encompasses mammals and non-mammals. Examples of mammals include, but are not limited to, humans, chimpanzees, apes, monkeys, cattle, horses, sheep, goats, swine; rabbits, dogs, cats, rats, mice, guinea pigs, and the like. Examples of non-mammals include, but are not limited to, birds, fish and the like. Frequently the subject is a human.

The term “a subject in need of such treatment”, refers to a subject which would benefit biologically, medically or in quality of life from such treatment.

As used herein, the terms “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically, (e.g., stabilization of a discernible symptom), physiologically, (e.g., stabilization of a physical parameter), or both.

As used herein, the term “prevent”, “preventing” or “prevention” of any disease or disorder refers to the prophylactic treatment of the disease or disorder; or delaying the onset or progression of the disease or disorder

The term “therapeutically effective amount” or “therapeutically effective dose” interchangeably refers to an amount sufficient to effect the desired result (i.e., reduction or inhibition of an enzyme or a protein activity, amelioration of symptoms, alleviation of symptoms or conditions, delay of disease progression, a reduction in tumor size, inhibition of tumor growth, prevention of metastasis, inhibition or prevention of viral, bacterial, fungal or parasitic infection). In some embodiments, a therapeutically effective amount does not induce or cause undesirable side effects. In some embodiments, a therapeutically effective amount induces or causes side effects but only those that are acceptable by the healthcare providers in view of a patient's condition. A therapeutically effective amount can be determined by first administering a low dose, and then incrementally increasing that dose until the desired effect is achieved. A “prophylactically effective dose” or a “prophylactically effect amount”, of the molecules of the invention can prevent the onset of disease symptoms, including symptoms associated with cancer. A “therapeutically effective dose” or a “therapeutically effective amount” of the molecules of the invention can result in a decrease in severity of disease symptoms, including symptoms associated with cancer.

The compound names provided herein were obtained using ChemBioDraw Ultra version 14.0.

As used herein, the term “a,” “an,” “the” and similar terms used in the context of the present invention (especially in the context of the claims) are to be construed to cover both the singular and plural unless otherwise indicated herein or clearly contradicted by the context.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen.

### Linker-Drug Group

The Linker-Drug group of the invention is a compound having the structure of Formula (I), or a pharmaceutically acceptable salt thereof:

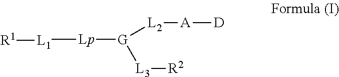

wherein:
R¹ is a reactive group;
L₁ is a bridging spacer;
Lp is a bivalent peptide spacer;
G-L₂-A is a self-immolative spacer;
R² is a hydrophilic moiety;
L₂ is a bond, a methylene, a neopentylene or a C₂-C₃alkenylene;
A is a bond, —OC(═O)—*,

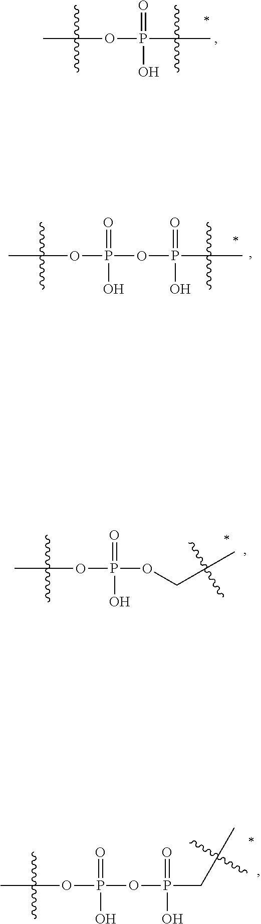 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
and
D is a Drug moiety comprising an N or an O, wherein D is connected to A via a direct bond from A to the N or the O of the Drug moiety.

Certain aspects and examples of the Linker-Drug group of the invention are provided in the following listing of enumerated embodiments. It will be recognized that features specified in each embodiment may be combined with other specified features to provide further embodiments of the present invention.
Embodiment 1. The compound of Formula (I), or pharmaceutically acceptable salt thereof, wherein:
   R¹ is a reactive group;
   L₁ is a bridging spacer;
   Lp is a bivalent peptide spacer comprising one to four amino acid residues;
   G-L₂-A is a self-immolative spacer;
   R² is a hydrophilic moiety;
   L₂ is a bond, a methylene, a neopentylene or a C₂-C₃alkenylene;
   A is a bond, —OC(═O)—*,

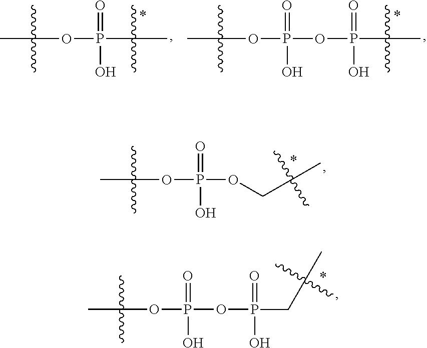 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
and
D is a Drug moiety comprising an N or an O, wherein D is connected to A via a direct bond from A to the N or the O of the Drug moiety.
Embodiment 2. The compound of Formula (I), or pharmaceutically acceptable salt thereof, wherein:
   R¹ is a reactive group;
   L₁ is a bridging spacer;
   Lp is a bivalent peptide spacer comprising one to four amino acid residues;
   the

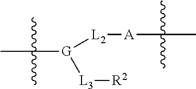

group is selected from:

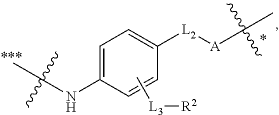

wherein the * of

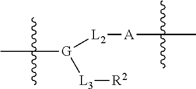

indicates the point of attachment to an N or a O of the Drug moiety, the *** of

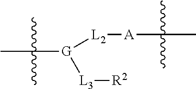

indicates the point of attachment to Lp;
R² is a hydrophilic moiety;
L₂ is a bond, a methylene, a neopentylene or a C₂-C₃alkenylene;
A is a bond, —OC(═O)—*,

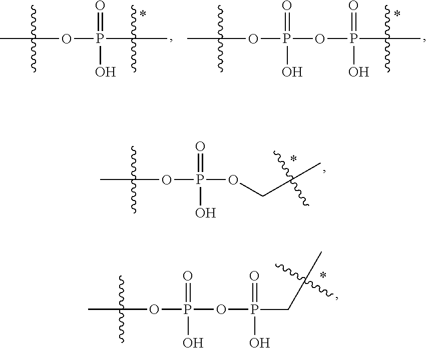 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
and
D is a Drug moiety comprising an N or an O, wherein D is connected to A via a direct bond from A to the N or the O of the Drug moiety.
Embodiment 3. The compound of Formula (I), having the structure of Formula (II):

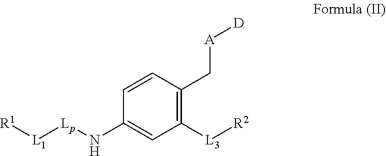

or pharmaceutically acceptable salt thereof,

wherein:
R¹ is a reactive group;
L₁ is a bridging spacer;
Lp is a bivalent peptide spacer comprising one to four amino acid residues;
R² is a hydrophilic moiety;
A is a bond, —OC(═O)—*,

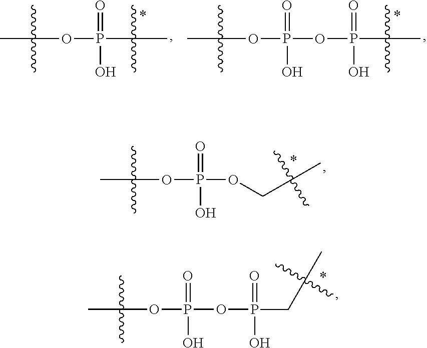 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
and
D is a Drug moiety comprising an N or an O, wherein D is connected to A via a direct bond from A to the N or the O of the Drug moiety.
Embodiment 4. The compound of Formula (I) or of any one of Embodiments 1 to 3, or pharmaceutically acceptable salt thereof, wherein:
   R¹ is

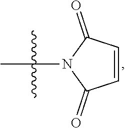 [SMILES: *N1C(=O)C=CC1=O]

—ONH₂, —NH₂,

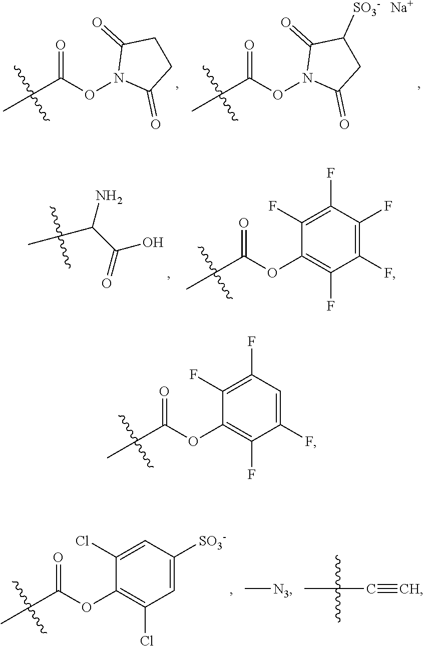

—SH, —SR³, —SSR⁴, —S(═O)(CH—CH₂), —(CH₂)₂S(═O)₂(CH═CH₂), —NHS(═O)₂(CH═CH₂), —NHC(═O)CH₂Br, —NHC(═O)CH₂I,

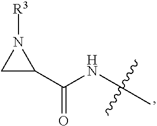

### —C(O)NHNH₂,

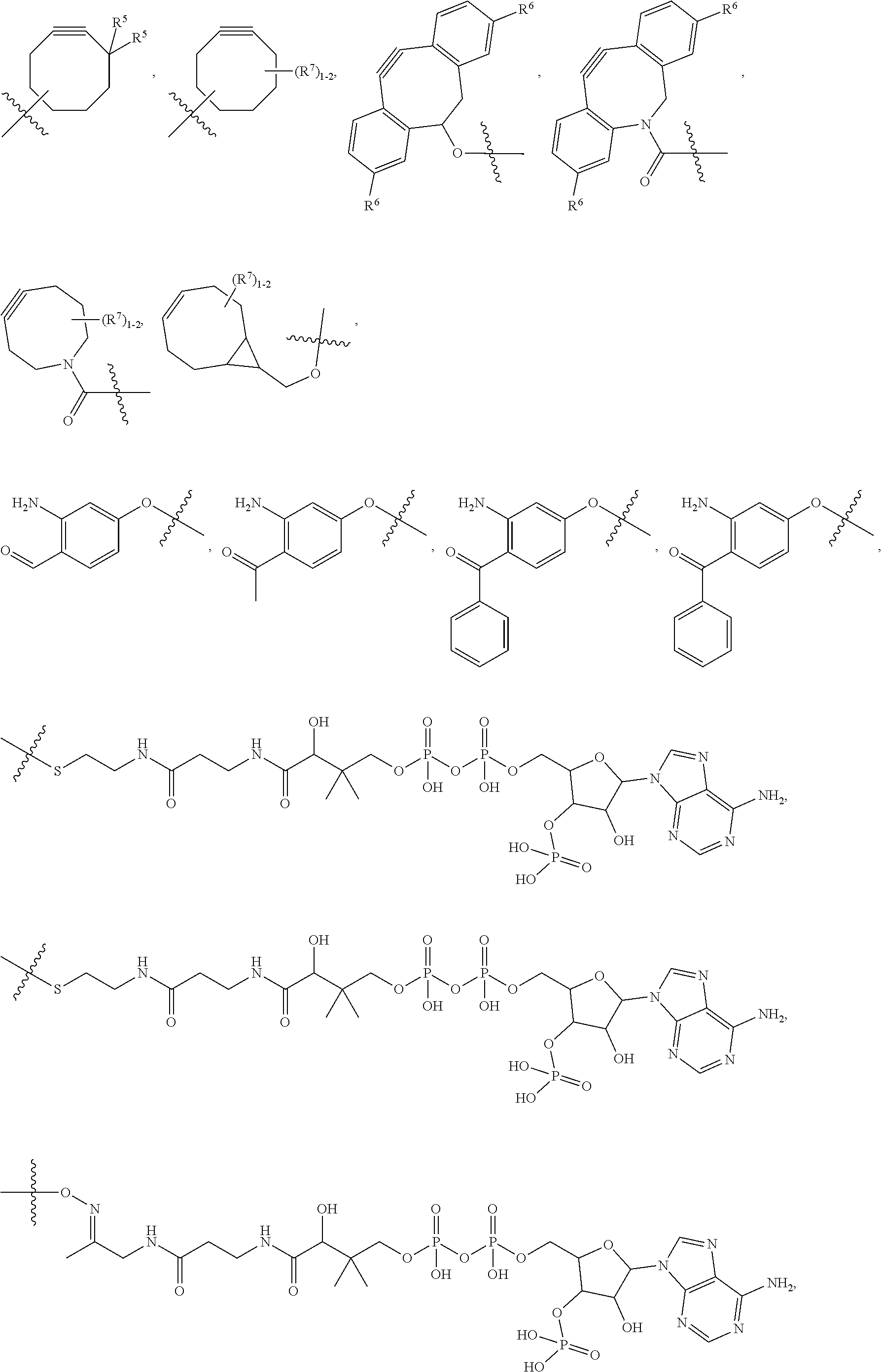 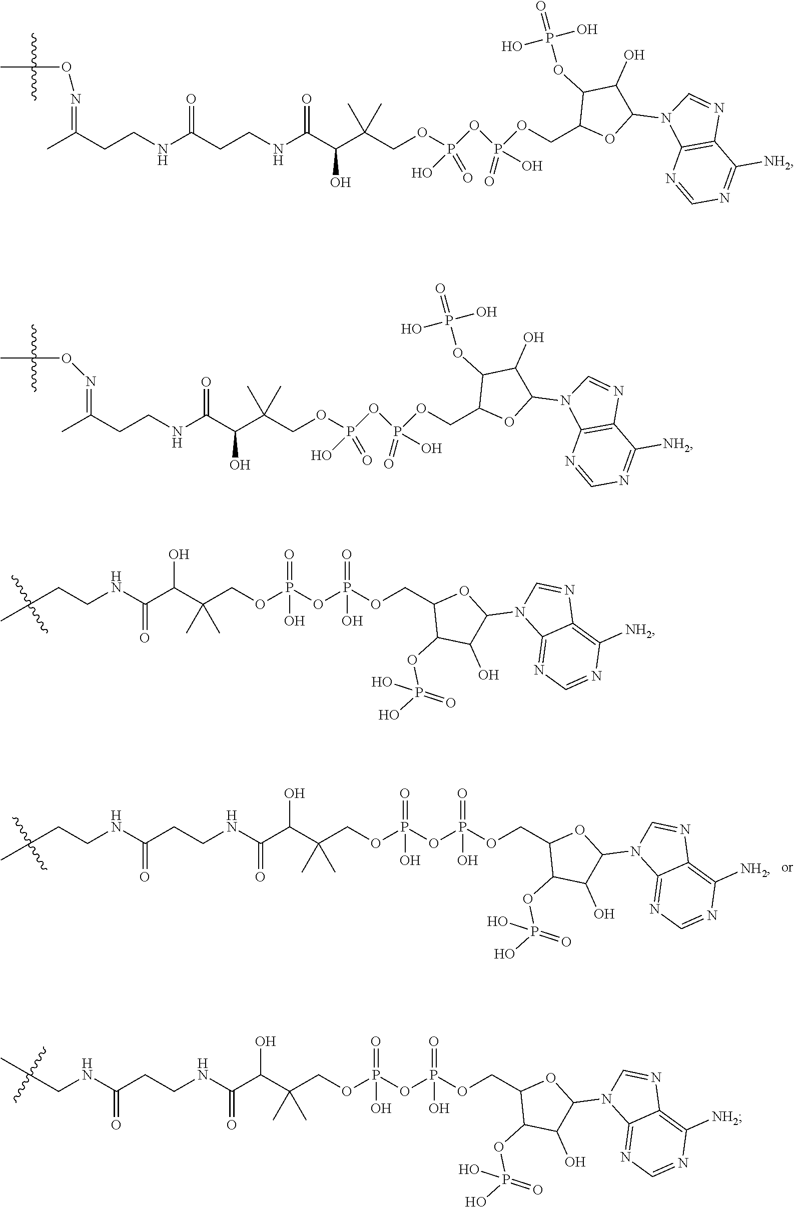 [SMILES: *O/N=C(\C)CCNC(=O)CCNC(=O)[C@H](O)C(C)(C)COP(=O)(O)OP(=O)(O)OCC1OC(n2cnc3c(N)ncnc32)C(O)C1OP(=O)(O)O ; *O/N=C(\C)CCNC(=O)[C@H](O)C(C)(C)COP(=O)(O)OP(=O)(O)OCC1OC(n2cnc3c(N)ncnc32)C(O)C1OP(=O)(O)O ; *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; "or" ; *CNC(=O)CCNC(=O)C(O)C(C)(C)COP(=O)(O)OP(=O)(O)OCC1OC(n2cnc3c(N)ncnc32)C(O)C1OP(=O)(O)O] [0238] L₁ is *—C(═O)(CH₂)ₘO(CH₂)ₘ—**; *—C(═O)((CH₂)ₘO)ₜ(CH₂)ₙ—**; *C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp, and the ** of L₁ indicates the point of attachment to R¹;
R² is a hydrophilic moiety selected from polyethylene glycol, polyalkylene glycol, a sugar, an oligosaccharide, a polypeptide or C₂-C₆alkyl substituted with 1 to 3

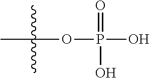 [SMILES: *OP(=O)(O)O]

groups;
each R³ is Independently selected from H and C₁-C₆alkyl;
R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —CN, —NO₂ and —OH;
each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
X₁ is

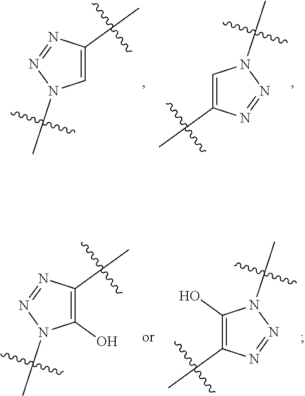 [SMILES: *c1cn(*)nn1 ; *c1cn(*)nn1 ; *c1nnn(*)c1O ; *c1nnn(*)c1O]

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from glycine, valine, citrulline, lysine, isoleucine, phenylalanine, methionine, asparagine, proline, alanine, leucine, tryptophan, and tyrosine;
A is a bond, —OC(═O)—*,

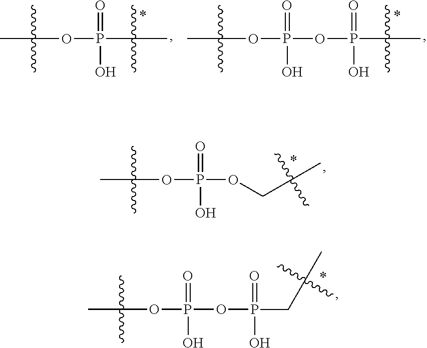 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety having the structure

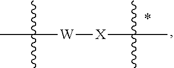

where
   (i) W is —CH₂O—**, —CH₂N(R^{b})C(═O)O—**, —NHC(═O)C(R^{b})₂NHC(═O)O—**, —NHC(═O)C(R^{b})₂NH—**, NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—**, —NH—, or —CH₂N(R^{b})C(═O)CH₂—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²; or
   (ii) W is —CH₂O—**, —CH₂N(R^{b})C(═O)O—**, —NHC(═O)C(R^{b})₂NHC(═O)O—**, —NHC(═O)C(R^{b})₂NH—**, NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is ***—CH₂-triazolyl-C₁₋₄ alkylene-OC(O)NHS(O)₂NH—*, ***—C₄₋₆ cycloalkylene-OC(O)NHS(O)₂NH—*, ***—(CH₂CH₂O),—C(O)NHS(O)₂NH—*, ***—(CH₂CH₂O)ₙ—C(O)NHS(O)₂NH—(CH₂CH₂O)ₙ—*, or ***—CH₂-triazolyl-C₁₋₄ alkylene-OC(O)NHS(O)₂NH—(CH₂CH₂O)ₙ—*, wherein each n independently is 1, 2, or 3, the *** of X indicates the point of attachment to W and the * of X indicates the point of attachment to R²;
and
the * of L₃ indicates the point of attachment to R²;
and
D is a Drug moiety comprising an N or an O, wherein D is connected to A via a direct bond from A to the N or the O of the Drug moiety.
Embodiment 5. The compound of Formula (I) or of any one of Embodiments 1 to 4, or pharmaceutically acceptable salt thereof, wherein:
   R¹ is

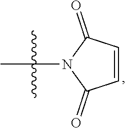 [SMILES: *N1C(=O)C=CC1=O]

### —ONH₂,

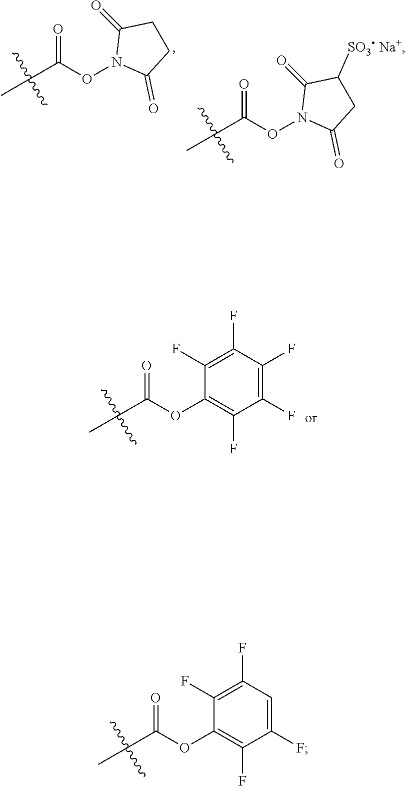 [0264] L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

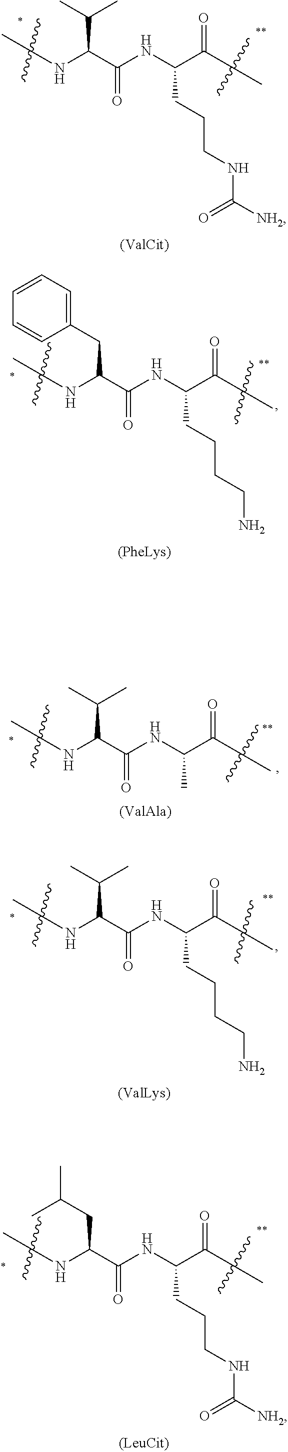

where the * of Lp indicates the attachment point to L₁ and the ** of Lp indicates the attachment point to the —NH— group of G;
L₃ is a spacer moiety having the structure

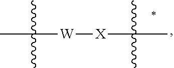

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH2-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

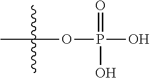 [SMILES: *OP(=O)(O)O]

groups;
A is a bond, —OC(═O)—*,

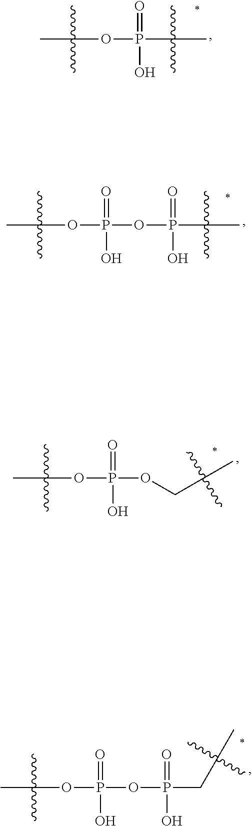 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
and
D is a Drug moiety comprising an N or an O, wherein D is connected to A via a direct bond from A to the N or the O of the Drug moiety.
Embodiment 6. The compound of Formula (I) or of any one of Embodiments 1 to 5, or pharmaceutically acceptable salt thereof, wherein:
   R¹ is

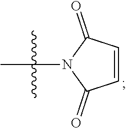 [SMILES: *N1C(=O)C=CC1=O]

L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

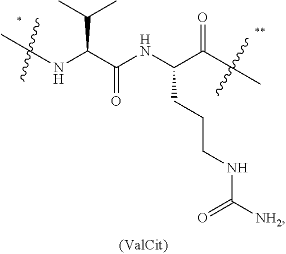 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

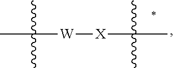

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH2-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

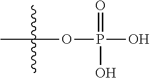 [SMILES: *OP(=O)(O)O]

groups;
A is a bond, —OC(═O)—*,

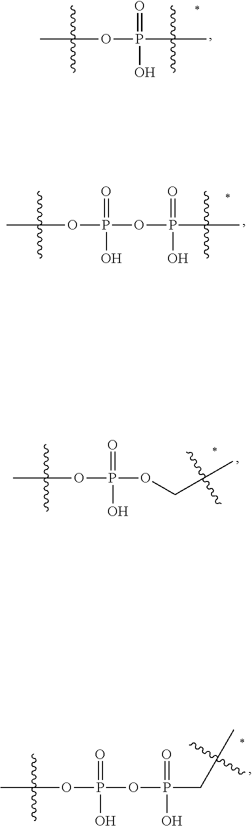 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
and
D is a Drug moiety comprising an N or an O, wherein D is connected to A via a direct bond from A to the N or the O of the Drug moiety.
Embodiment 7. The compound of Formula (I) or of any one of Embodiments 1 to 6, or pharmaceutically acceptable salt thereof, wherein:
   R¹ is

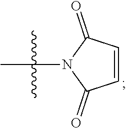 [SMILES: *N1C(=O)C=CC1=O]

L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

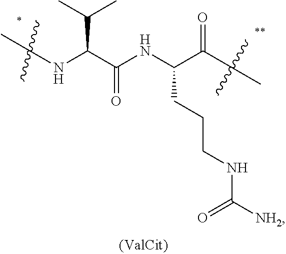 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

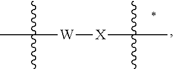

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
   X is a bond, triazolyl or ***—CH2-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

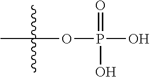 [SMILES: *OP(=O)(O)O]

groups;
A is a bond or —OC(═O)*, in which * indicates the attachment point to D;
and
D is a Drug moiety comprising an N or an O, wherein D is connected to A via a direct bond from A to the N or the O of the Drug moiety.
Embodiment 8. The compound of Formula (I) or of any one of Embodiments 1 to 7, or pharmaceutically acceptable salt thereof, wherein:
   R¹ is

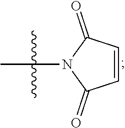 [SMILES: *N1C(=O)C=CC1=O]

L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

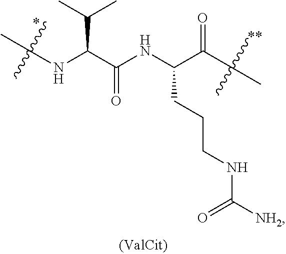 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

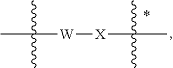

where
   W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

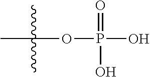 [SMILES: *OP(=O)(O)O]

groups;
A is a bond or —OC(═O)*, in which * indicates the attachment point to D;
and
D is a Drug moiety comprising an N or an O, wherein D is connected to A via a direct bond from A to the N or the O of the Drug moiety.
Embodiment 9. The compound of Formula (I) or of any one of Embodiments 1 to 8, or pharmaceutically acceptable salt thereof, wherein R¹ is a reactive group selected from Table 1 or Table 2.
Embodiment 10. The compound of Formula (I) or of any one of Embodiments 1 to 9, or pharmaceutically acceptable salt thereof, wherein:
   R¹ is

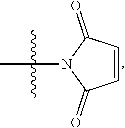 [SMILES: *N1C(=O)C=CC1=O]

—ONH₂, —NH₂,

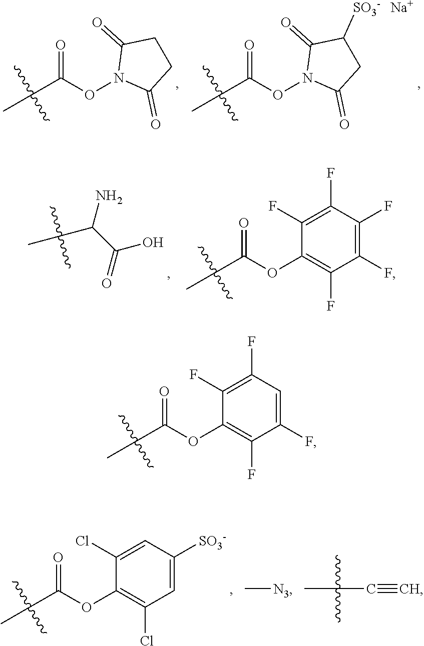

—SH, —SR³, —SSR⁴, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I,

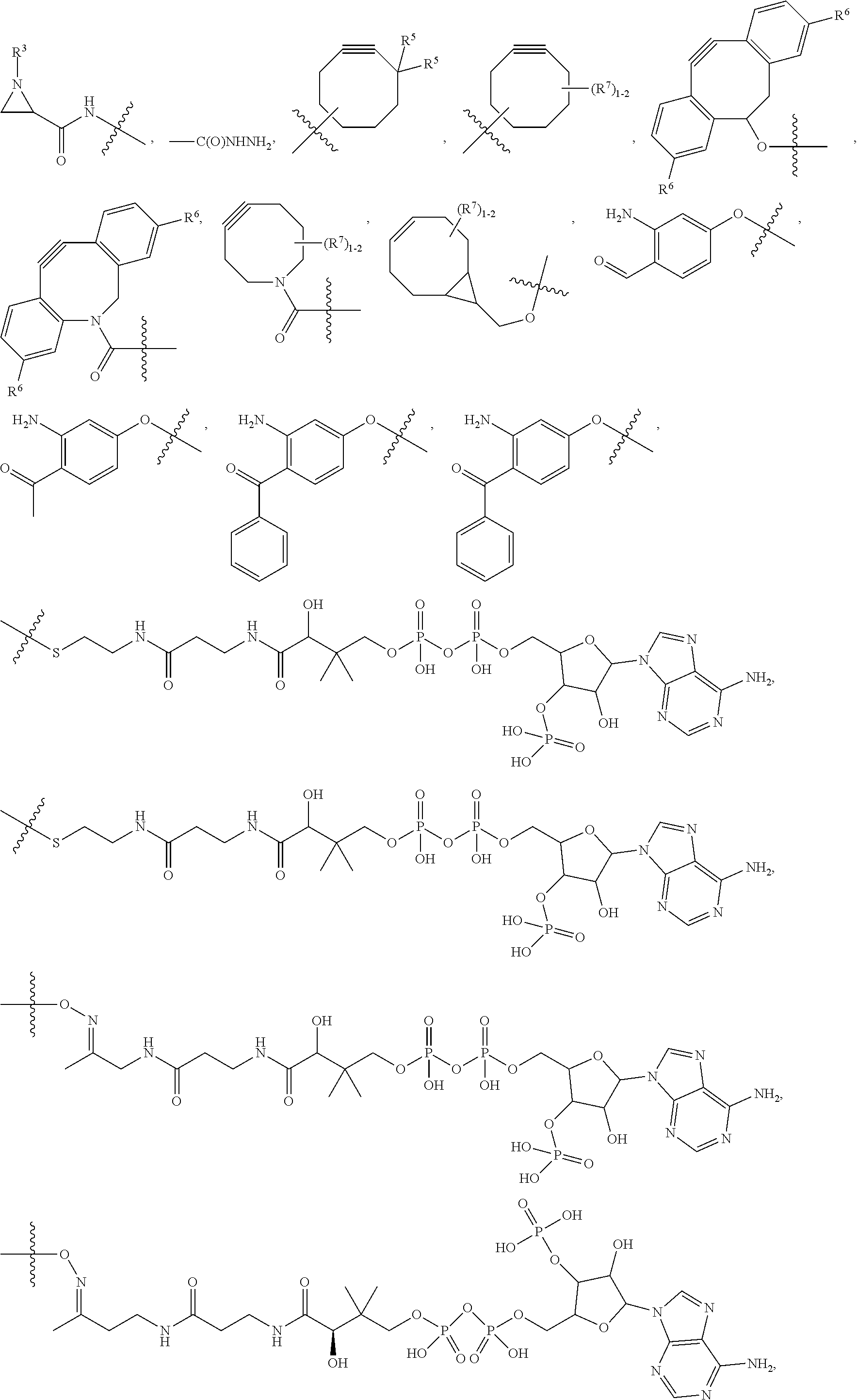

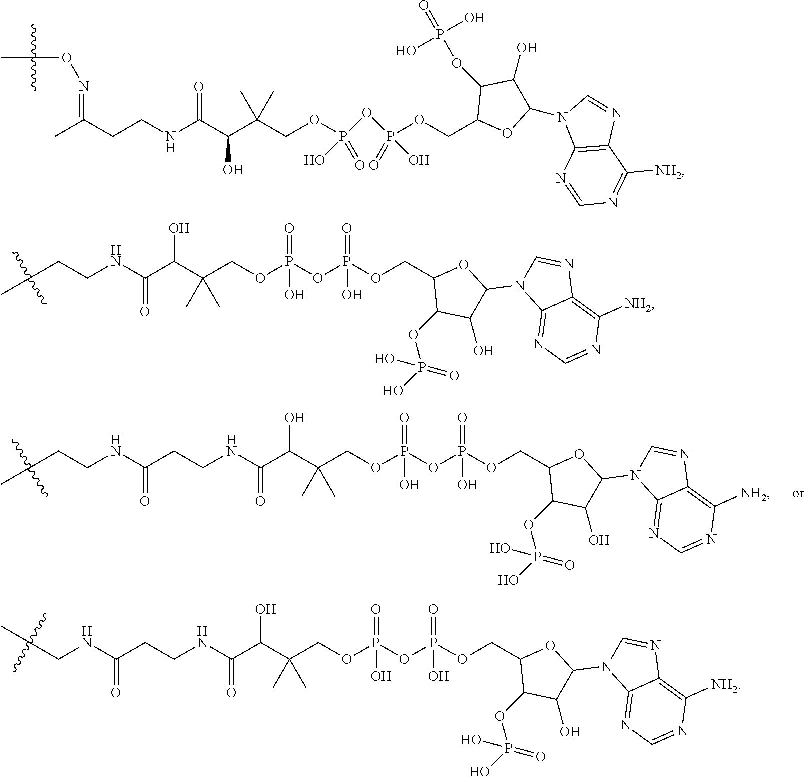

Embodiment 11. The compound of Formula (I) or of any one of Embodiments 1 to 9, or pharmaceutically acceptable salt thereof, wherein:
   R¹ is

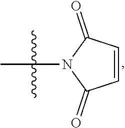 [SMILES: *N1C(=O)C=CC1=O]

—ONH₂, —NH₂,

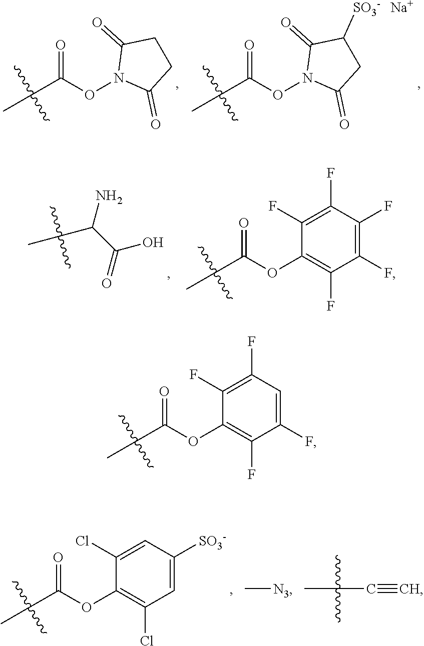

—SH, —SR³, —SSR⁴, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I,

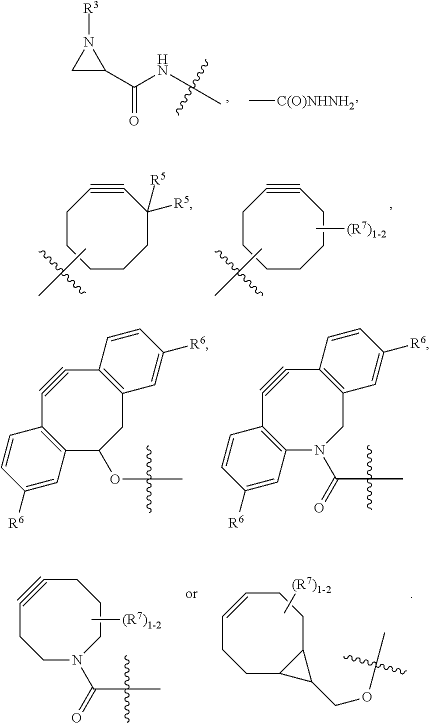

Embodiment 12. The compound of Formula (I) or of any one of Embodiments 1 to 9, or pharmaceutically acceptable salt thereof, wherein:
   R¹ is

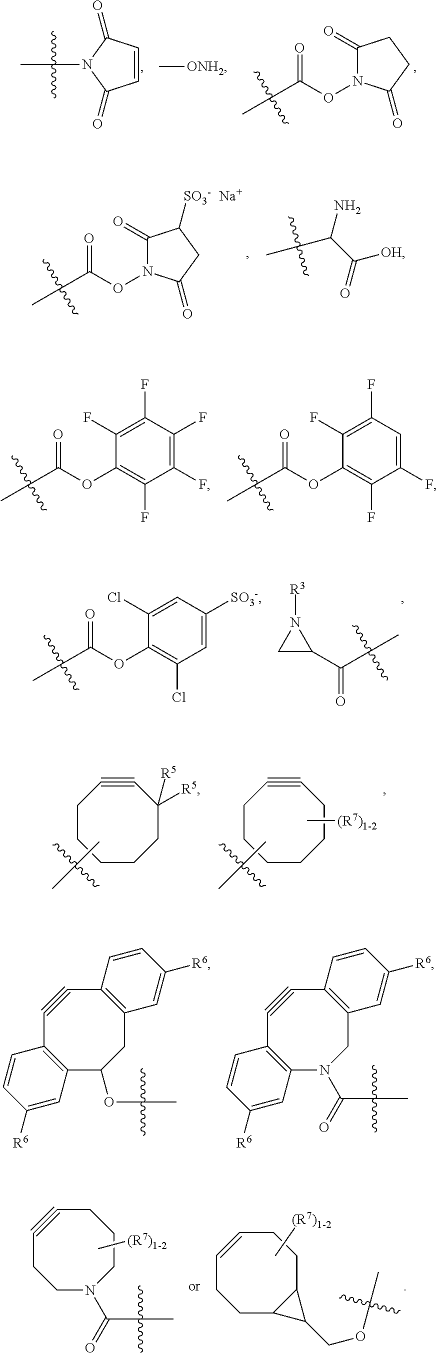

Embodiment 13. The compound of Formula (I) or of any one of Embodiments 1 to 9, or a pharmaceutically acceptable salt thereof, wherein:
   R¹ is

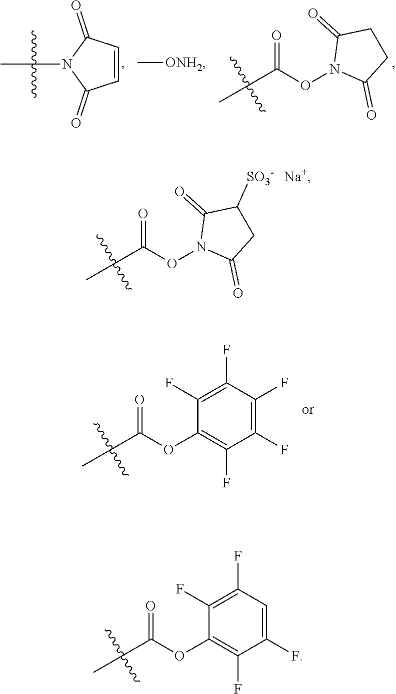

Embodiment 14. The compound of Formula (I) or of any one of Embodiments 1 to 9, or a pharmaceutically acceptable salt thereof, wherein R₁ is

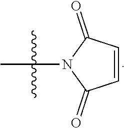 [SMILES: *N1C(=O)C=CC1=O]

Embodiment 15. The compound of Formula (I) or of any one of Embodiments 1 to 9, or a pharmaceutically acceptable salt thereof, wherein R¹ is —ONH₂.
Embodiment 16. The compound of Formula (I) or of any one of Embodiments 1 to 9, or a pharmaceutically acceptable salt thereof, wherein: R¹ is

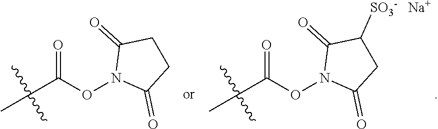

Embodiment 17. The compound of Formula (I) or of any one of Embodiments 1 to 9, or pharmaceutically acceptable salt thereof, wherein:
   R¹ is

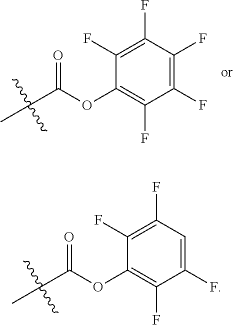 [SMILES: *C(=O)Oc1c(F)c(F)c(F)c(F)c1F ; *C(=O)Oc1c(F)c(F)cc(F)c1F]

Embodiment 18. The compound of Formula (I) or of any one of Embodiments 1 to 8, having the structure:

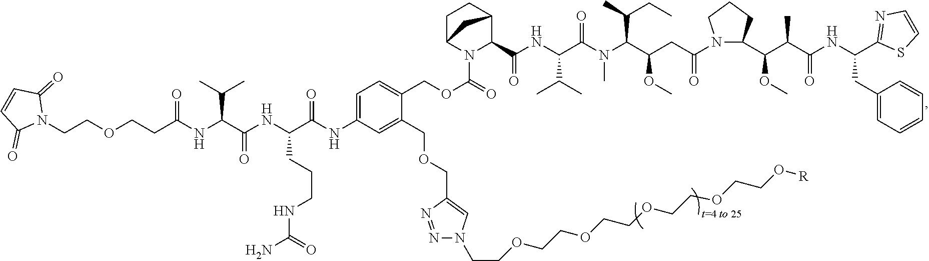

or a pharmaceutically acceptable salt thereof, where R is H, —CH₃ or —CH₂CH₂C(═O)OH.
Embodiment 19. The compound of Formula (I) or of any one of Embodiments 1 to 8, having the structure:

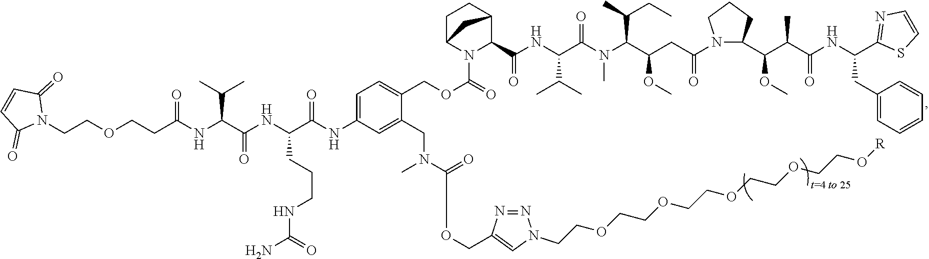

Embodiment 20. The compound of Formula (I) or of any one of Embodiments 1 to 8, having the structure:

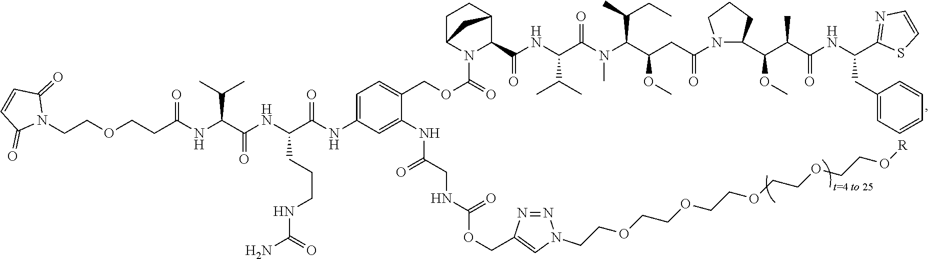

or a pharmaceutically acceptable salt thereof, where R is H, —CH₃ or —CH₂CH₂C(═O)OH.
Embodiment 21. The compound of Formula (I) or of any one of Embodiments 1 to 8, having the structure:

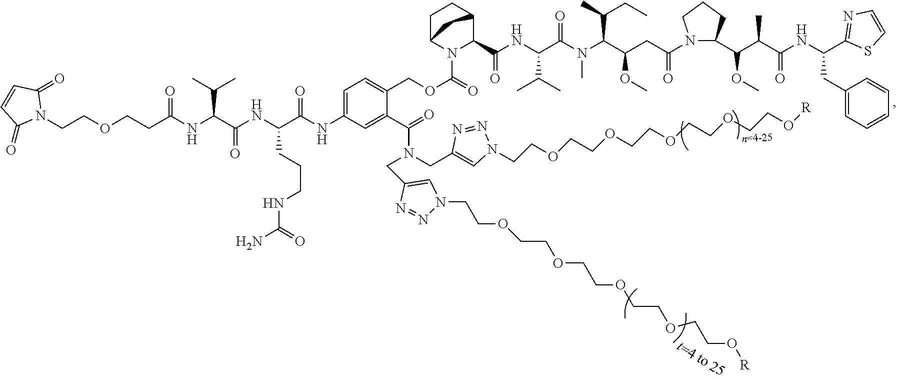

for a pharmaceutically acceptable salt thereof, where each R is independently selected from H, —CH₃ or —CH₂CH₂C(═O)OH.
Embodiment 22. The compound of Formula (I) or of any one of Embodiments 1 to 8, having the structure:

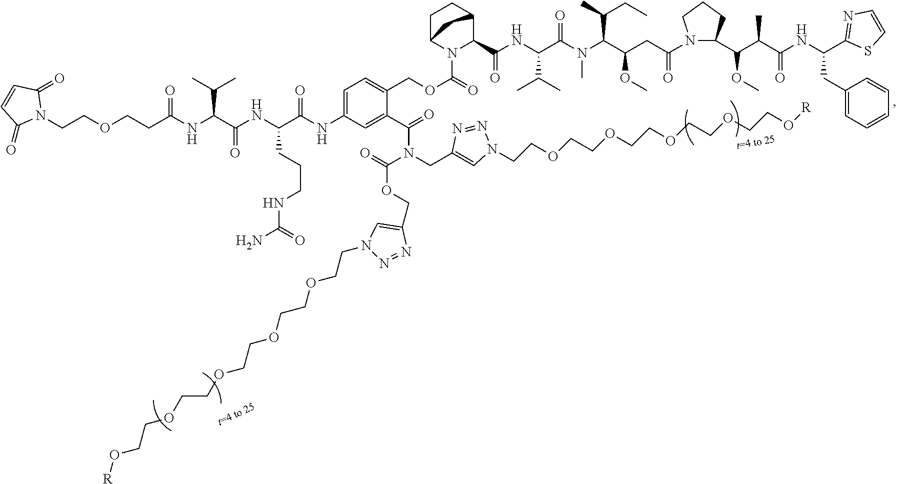

or a pharmaceutically acceptable salt thereof, where each R is independently selected from H, —CH₃ or —CH₂CH₂C(═O)OH.
Embodiment 23. The compound of Formula (I) or of any one of Embodiments 1 to 8, having the structure:

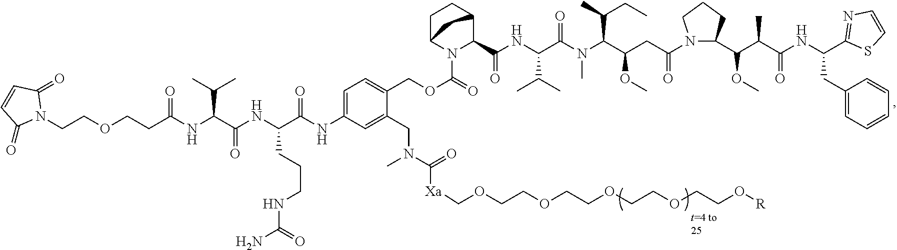

or a pharmaceutically acceptable salt thereof, where Xa is —CH₂—, —OCH₂—, —NHCH₂— or —NRCH₂— and each R independently is H, —CH₃ or —CH₂CH₂C(═O)OH.
Embodiment 24. The compound of Formula (I) or of any one of Embodiments 1 to 8, having the structure:

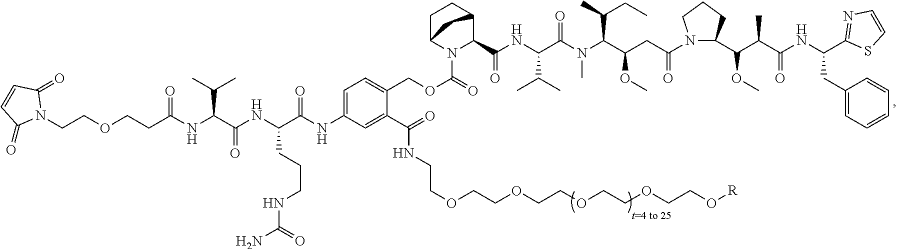

or a pharmaceutically acceptable salt thereof, where R is H, —CH₃ or —CH₂CH₂C(═O)OH.
Embodiment 25. The compound of Formula (I) or of any one of Embodiments 1 to 8, having the structure:

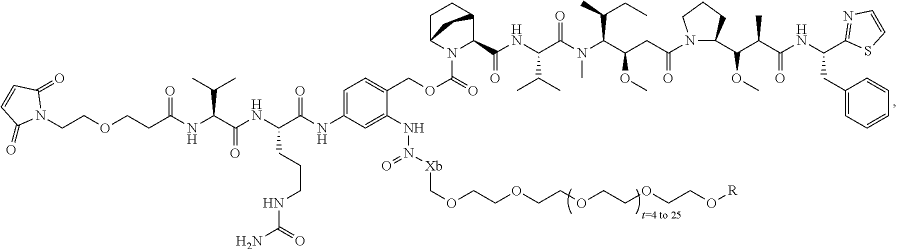

or a pharmaceutically acceptable salt thereof, where Xb is —CH₂—, —OCH₂—, —NHCH₂— or —NRCH₂— and each R independently is H, —CH₃ or —CH₂CH₂C(═O)OH.
Embodiment 26. The compound of Formula (I) or of any one of Embodiments 1 to 8, having the structure:

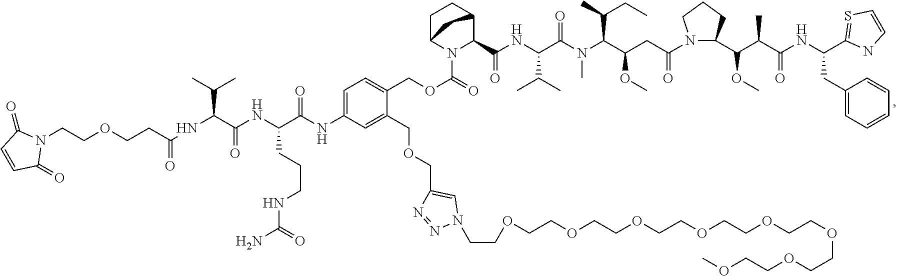

or a pharmaceutically acceptable salt thereof.
Embodiment 27. The compound of Formula (I) or of any one of Embodiments 1 to 8, having the structure:

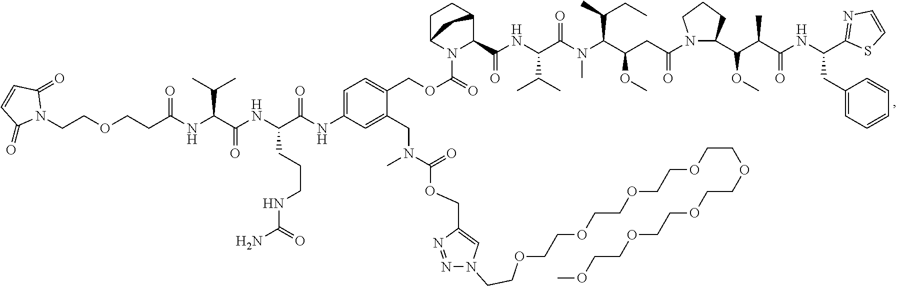

or a pharmaceutically acceptable salt thereof.
Embodiment 28. The compound of Formula (I) or of any one of Embodiments 1 to 8, having the structure:

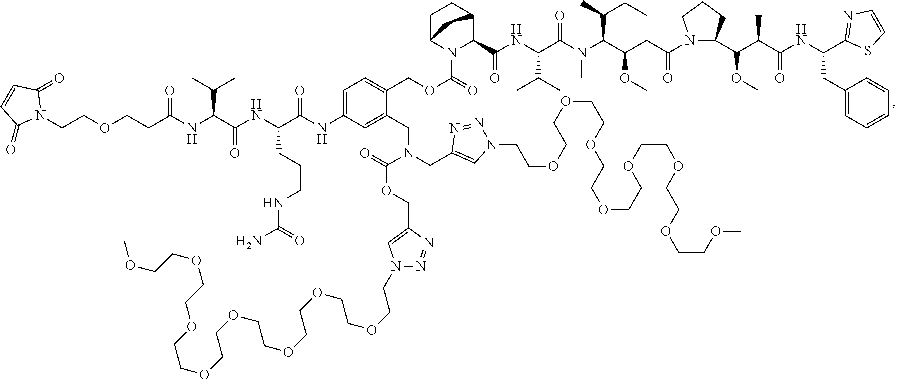

or pharmaceutically acceptable salt thereof.
Embodiment 29. The compound of Formula (I) or of any one of Embodiments 1 to 8, having the structure:

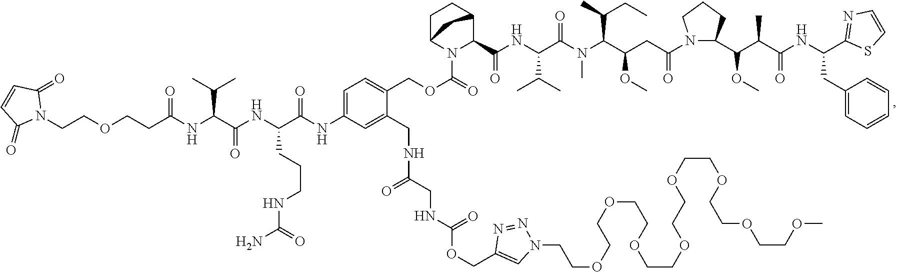

or pharmaceutically acceptable salt thereof.
Embodiment 30. The compound of Formula (I) or of any one of Embodiments 1 to 8, having the structure:

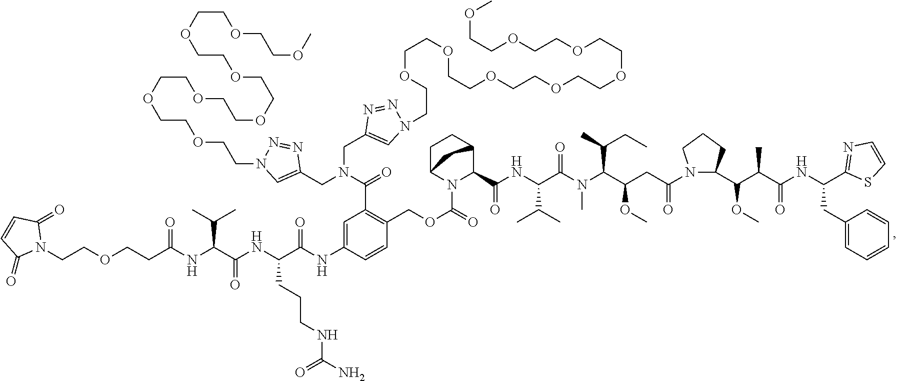

or a pharmaceutically acceptable salt thereof.
Embodiment 31. The compound of Formula (I) or of any one of Embodiments 1 to 8, or pharmaceutically acceptable salt thereof, having the structure of a compound in any of Tables 4A-4C included herein.
Embodiment 32. A linker of the Linker-Drug group of Formula (I) having the structure of Formula (V),

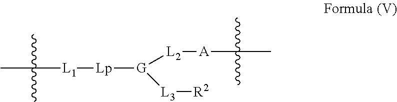

wherein
L₁ is a bridging spacer;
Lp is a bivalent peptide spacer;
G-L₂-A is a self-immolative spacer;
R² is a hydrophilic moiety;
L₂ is a bond, a methylene, a neopentylene or a C₂-C₃alkenylene;
A is a bond, **—OC(═O)—,

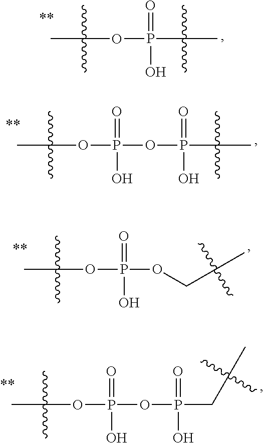 [SMILES: *OP(*)(=O)O ; *OP(=O)(O)OP(*)(=O)O ; *COP(=O)(O)O* ; *CP(=O)(O)OP(=O)(O)O*]

**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂,
and
L₃ is a spacer moiety.
Embodiment 33. The linker of Embodiment 32, wherein:
   L₁ is a bridging spacer;
   Lp is a bivalent peptide spacer comprising one to four amino acid residues;
   G-L₂-A is a self-immolative spacer;
   R² is a hydrophilic moiety;
   L₂ is a bond, a methylene, a neopentylene or a C₂-C₃alkenylene;
   A is a bond, **—OC(═O)—,

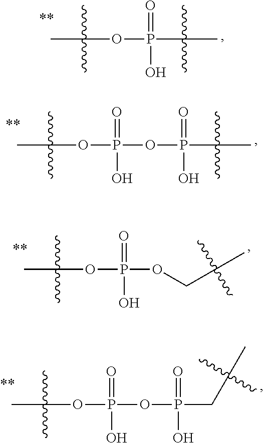 [SMILES: *OP(*)(=O)O ; *OP(=O)(O)OP(*)(=O)O ; *COP(=O)(O)O* ; *CP(=O)(O)OP(=O)(O)O*]

**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂,
and
L₃ is a spacer moiety.
Embodiment 34. The linker of Embodiment 32 or 33, wherein:
   L₁ is a bridging spacer;
   Lp is a bivalent peptide spacer comprising one to four amino acid residues;
   the

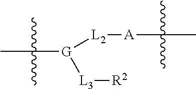

group is selected from:

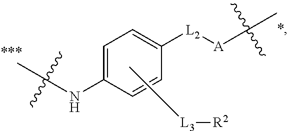

wherein the * of

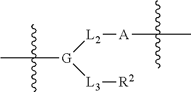

indicates the point of attachment to an N or O of the Drug moiety, the *** of

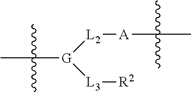

indicates the point of attachment to Lp;
R² is a hydrophilic moiety;
L₂ is a bond, a methylene, a neopentylene or a C₁-C₃alkenylene;
A is a bond, **—OC(═O)—,

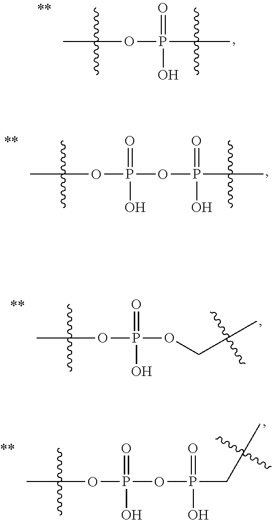 [SMILES: *OP(*)(=O)O ; *OP(=O)(O)OP(*)(=O)O ; *COP(=O)(O)O* ; *CP(=O)(O)OP(=O)(O)O*]

**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂,
and
L₃ is a spacer moiety.
Embodiment 35. The linker of any one of Embodiments 32 to 34, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp;
   R² is a hydrophilic moiety selected from polyethylene glycol, polyalkylene glycol, a sugar, an oligosaccharide, a polypeptide or C₂-C₆alkyl substituted with 1 to 3

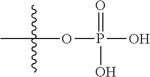 [SMILES: *OP(=O)(O)O]

groups;
each R³ is independently selected from H and C₁-C₆alkyl;
X₁ is

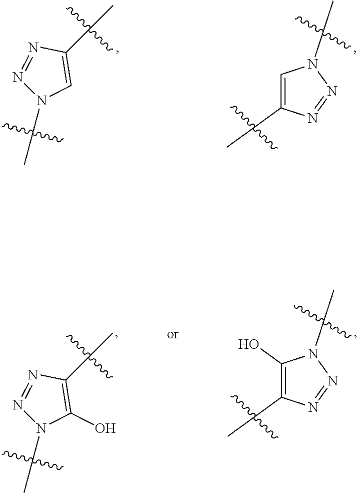 [SMILES: *c1cn(*)nn1 ; *c1cn(*)nn1 ; *c1nnn(*)c1O ; *c1nnn(*)c1O]

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from glycine, valine, citrulline, lysine, isoleucine, phenylalanine, methionine, asparagine, proline, alanine, leucine, tryptophan, and tyrosine;
A is a bond, **—OC(═O)—,

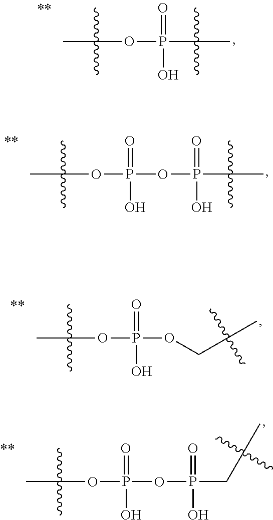 [SMILES: *OP(*)(=O)O ; *OP(=O)(O)OP(*)(=O)O ; *COP(=O)(O)O* ; *CP(=O)(O)OP(=O)(O)O*]

**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂;
L₃ is a spacer moiety having the structure

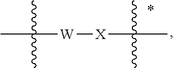

where
   (i) W is —CH₂O—**, —CH₂N(R^{b})C(═O)O—**, —NHC(═O)C(R^{b})₂NHC(═O)O—**, —NHC(═O)C(R^{b})₂NH—**, NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—**, —NH—, or —CH₂N(R^{b})C(═O)CH₂—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²; or
   (ii) W is —CH₂O—**, —CH₂N(R^{b})C(═O)O—**, —NHC(═O)C(R^{b})₂NHC(═O)O—**, —NHC(═O)C(R^{b})₂NH—**, NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is ***—CH₂-triazolyl-C₁₋₄ alkylene-OC(O)NHS(O)₂NH—*, ***—C₄₋₆ cycloalkylene-OC(O)NHS(O)₂NH—*, ***—(CH₂CH₂O),—C(O)NHS(O)₂NH—*, ***—(CH₂CH₂O)ₙ—C(O)NHS(O)₂NH—(CH₂CH₂O)ₙ—*, or ***—CH₂-triazolyl-C₁₋₄ alkylene-OC(O)NHS(O)₂NH—(CH₂CH₂O)ₙ—*, wherein each n independently is 1, 2, or 3, the *** of X indicates the point of attachment to W and the * of X indicates the point of attachment to R²;
and
   the * of L₃ indicates the point of attachment to R².
Embodiment 36. The linker of any one of Embodiments 32 to 35, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
   each m is independently selected from 1, 2, 3, 4, 5, 6, 7, 8, 9 and 10;
   each n is independently selected from 1, 2, 3, 4, 5, 6, 7, 8, 9 and 10;
   each t is independently selected from 6, 7, 8, 9, 10, 11, 12, 13, 14, 15, 16, 17, 18, 19, 20, 21, 22, 23, 24, 25, 26, 27, 28, 29 and 30;
   Lp is a bivalent peptide spacer selected from

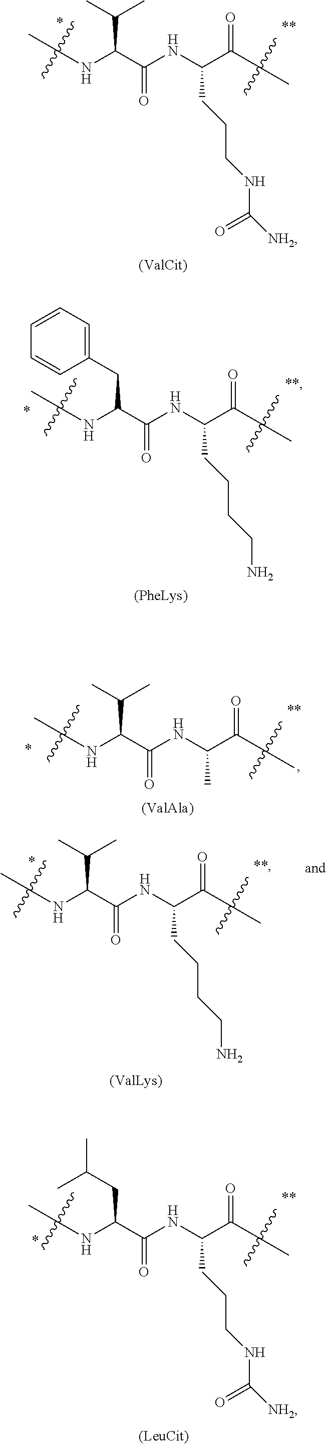

where the * of Lp indicates the attachment point to L₁ and the ** of Lp indicates the attachment point to the —NH— group of G;
L₃ is a spacer moiety having the structure

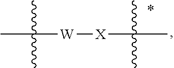

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

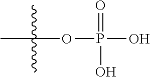 [SMILES: *OP(=O)(O)O]

groups;
and
A is a bond, **—OC(═O)—,

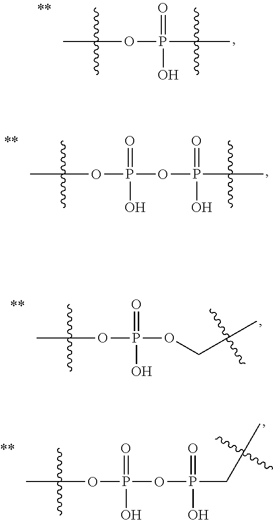 [SMILES: *OP(*)(=O)O ; *OP(=O)(O)OP(*)(=O)O ; *COP(=O)(O)O* ; *CP(=O)(O)OP(=O)(O)O*]

**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂.
Embodiment 37. The linker of any one of Embodiments 32 to 36, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ, where the * of L₁ indicates the point of attachment to Lp;
   each m is independently selected from 1, 2, 3, 4, 5, 6, 7, 8, 9 and 10;
   each n is independently selected from 1, 2, 3, 4, 5, 6, 7, 8, 9 and 10;
   each t is independently selected from 6, 7, 8, 9, 10, 11, 12, 13, 14, 15, 16, 17, 18, 19, 20, 21, 22, 23, 24, 25, 26, 27, 28, 29 and 30;
   Lp is a bivalent peptide spacer selected from

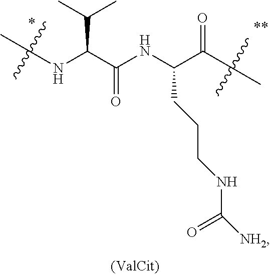 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

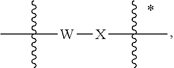

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH— wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

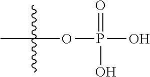 [SMILES: *OP(=O)(O)O]

groups;
and
A is a bond, **—OC(═O)—,

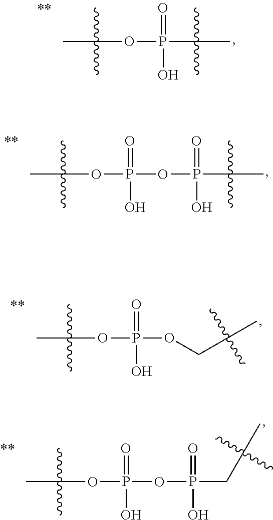 [SMILES: *OP(*)(=O)O ; *OP(=O)(O)OP(*)(=O)O ; *COP(=O)(O)O* ; *CP(=O)(O)OP(=O)(O)O*]

**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂.
Embodiment 38. The linker of any one of Embodiments 32 to 37, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
   each m is independently selected from 1, 2, 3, 4, 5, 6, 7, 8, 9 and 10;
   each n is independently selected from 1, 2, 3, 4, 5, 6, 7, 8, 9 and 10;
   each t is independently selected from 6, 7, 8, 9, 10, 11, 12, 13, 14, 15, 16, 17, 18, 19, 20, 21, 22, 23, 24, 25, 26, 27, 28, 29 and 30;
   Lp is a bivalent peptide spacer selected from

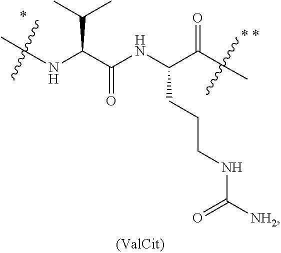 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

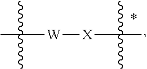

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

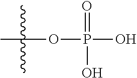 [SMILES: *OP(=O)(O)O]

groups;
and
A is a bond or **—OC(═O)—, wherein the ** of A indicates the point of attachment to L₂.
Embodiment 39. The linker of any one of Embodiments 32 to 38, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
   each m is independently selected from 1, 2, 3, 4, 5, 6, 7, 8, 9 and 10;
   each n is independently selected from 1, 2, 3, 4, 5, 6, 7, 8, 9 and 10;
   each t is independently selected from 6, 7, 8, 9, 10, 11, 12, 13, 14, 15, 16, 17, 18, 19, 20, 21, 22, 23, 24, 25, 26, 27, 28, 29 and 30;
   Lp is a bivalent peptide spacer selected from

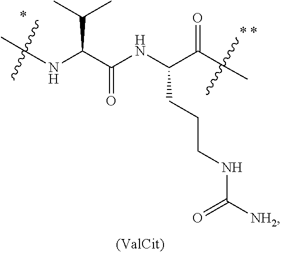 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

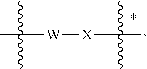

where
   W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

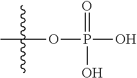 [SMILES: *OP(=O)(O)O]

groups;
and
A is a bond or **—OC(═O)—, wherein the ** of A indicates the point of attachment to L₂.
Embodiment 40. The linker of Formula (V) having the structure having the structure of Formula (VI),

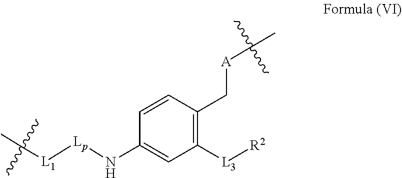

wherein
L₁ is a bridging spacer;
Lp is a bivalent peptide spacer;
R² is a hydrophilic moiety;
A is a bond, —OC(═O)—,

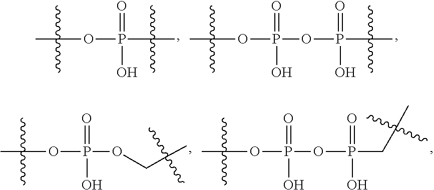

—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl,
and
L₃ is a spacer moiety.
Embodiment 41. The linker of Embodiments 40, wherein:
   L₁ is a bridging spacer;
   Lp is a bivalent peptide spacer comprising one to four amino acid residues;
   R² is a hydrophilic moiety;
   A is a bond, —OC(═O)—,

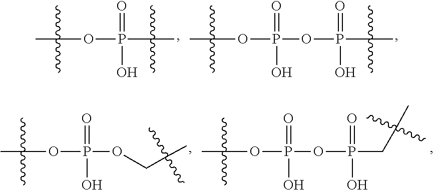

—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl,
and
L₃ is a spacer moiety.
Embodiment 42. The linker of Embodiment 40 or 41, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp;
   R² is a hydrophilic moiety selected from polyethylene glycol, polyalkylene glycol, a sugar, an oligosaccharide, a polypeptide, C₂-C₆alkyl substituted with 1 to 3

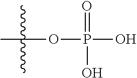 [SMILES: *OP(=O)(O)O]

groups, or a polysarcosine;
each R³ is independently selected from H and C₁-C₆alkyl;
X₁ is

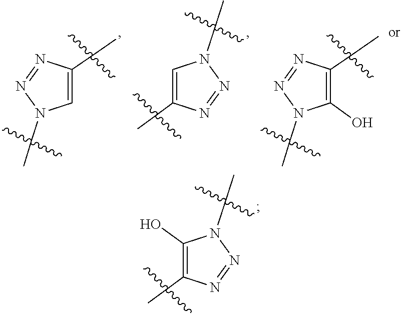

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from glycine, valine, citrulline, lysine, isoleucine, phenylalanine, methionine, asparagine, proline, alanine, leucine, tryptophan, and tyrosine;
A is a bond, —OC(═O)—,

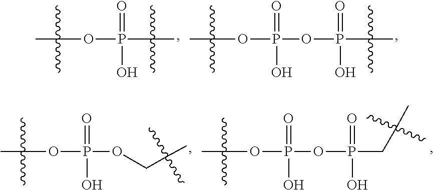

—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl;
L₃ is a spacer moiety having the structure

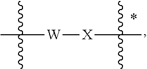

where
   (i) W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—**, —NH—, or —CH₂N(R^{b})C(═O)CH₂—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²; or
   (ii) W is —CH₂O—**, —CH₂N(R^{b})C(═O)O—**, —NHC(═O)C(R^{b})₂NHC(═O)O—**, —NHC(═O)C(R^{b})₂NH—**, NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is ***—CH₂-triazolyl-C₁₋₄ alkylene-OC(O)NHS(O)₂NH—*, ***—C₄₋₆ cycloalkylene-OC(O)NHS(O)₂NH—*, ***—(CH₂CH₂O),—C(O)NHS(O)₂NH—*, ***—(CH₂CH₂O)ₙ—C(O)NHS(O)₂NH—(CH₂CH₂O)ₙ—*, or ***—CH₂-triazolyl-C₁₋₄ alkylene-OC(O)NHS(O)₂NH—(CH₂CH₂O)ₙ—*, wherein each n independently is 1, 2, or 3, the *** of X indicates the point of attachment to W and the * of X indicates the point of attachment to R²;
   and
   the * of L₃ indicates the point of attachment to R².
Embodiment 43. The linker of any one of Embodiments 40 to 42, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
   each m is independently selected from 1, 2, 3, 4, 5, 6, 7, 8, 9 and 10;
   each n is independently selected from 1, 2, 3, 4, 5, 6, 7, 8, 9 and 10;
   each t is independently selected from 6, 7, 8, 9, 10, 11, 12, 13, 14, 15, 16, 17, 18, 19, 20, 21, 22, 23, 24, 25, 26, 27, 28, 29 and 30;
   Lp is a bivalent peptide spacer selected from

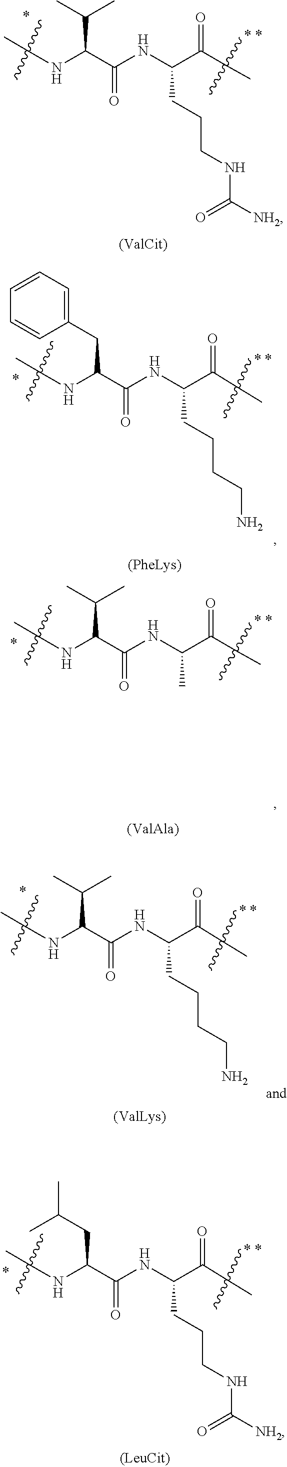

where the * of Lp indicates the attachment point to L₁ and the ** of Lp indicates the attachment point to the —NH— group;
L₃ is a spacer moiety having the structure

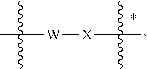

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

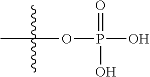 [SMILES: *OP(=O)(O)O]

groups, or a polysarcosine;
and
A is a bond, —OC(═O)—,

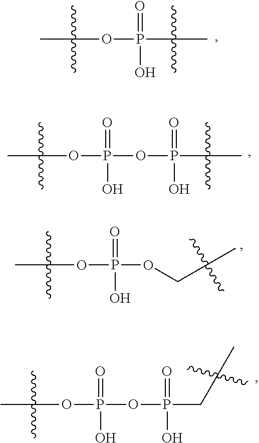

—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl.
Embodiment 44. The linker of any one of Embodiments 40 to 43, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ, where the * of L₁ indicates the point of attachment to Lp;
   each m is independently selected from 1, 2, 3, 4, 5, 6, 7, 8, 9 and 10;
   each n is independently selected from 1, 2, 3, 4, 5, 6, 7, 8, 9 and 10;
   each t is independently selected from 6, 7, 8, 9, 10, 11, 12, 13, 14, 15, 16, 17, 18, 19, 20, 21, 22, 23, 24, 25, 26, 27, 28, 29 and 30;

Lp is a bivalent peptide spacer selected from

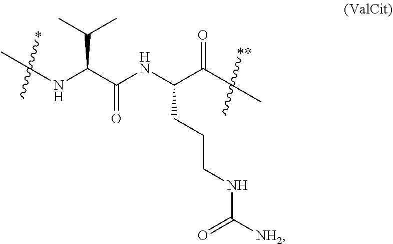

where the * of Lp indicates the attachment point to L₁ and the ** of Lp indicates the attachment point to the —NH— group;
L₃ is a spacer moiety having the structure

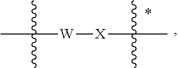

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

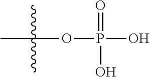 [SMILES: *OP(=O)(O)O]

groups or a polysarcosine;
and
A is a bond, —OC(═O)—,

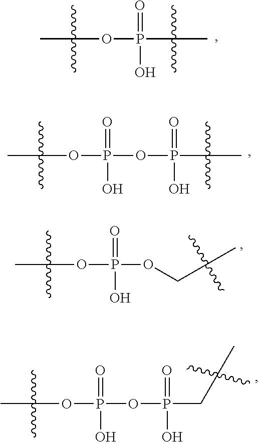

—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl.
Embodiment 45. The linker of any one of Embodiments 40 to 44, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
   each m is independently selected from 1, 2, 3, 4, 5, 6, 7, 8, 9 and 10;
   each n is independently selected from 1, 2, 3, 4, 5, 6, 7, 8, 9 and 10;
   each t is independently selected from 6, 7, 8, 9, 10, 11, 12, 13, 14, 15, 16, 17, 18, 19, 20, 21, 22, 23, 24, 25, 26, 27, 28, 29 and 30;

Lp is a bivalent peptide spacer selected from

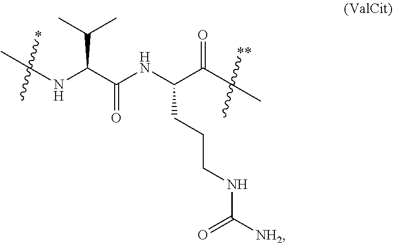

where the * of Lp indicates the attachment point to L₁ and the ** of Lp indicates the attachment point to the —NH— group;
L₃ is a spacer moiety having the structure

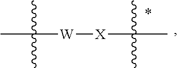

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

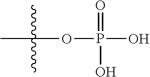 [SMILES: *OP(=O)(O)O]

groups, or a polysarcosine;
and
A is a bond or —OC(═O)—.
Embodiment 46. The linker of any one of Embodiments 40 to 45, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ, where the * of L₁ indicates the point of attachment to Lp;
   each m is independently selected from 1, 2, 3, 4, 5, 6, 7, 8, 9 and 10;
   each n is independently selected from 1, 2, 3, 4, 5, 6, 7, 8, 9 and 10;
   each t is independently selected from 6, 7, 8, 9, 10, 11, 12, 13, 14, 15, 16, 17, 18, 19, 20, 21, 22, 23, 24, 25, 26, 27, 28, 29 and 30;
   Lp is a bivalent peptide spacer selected from

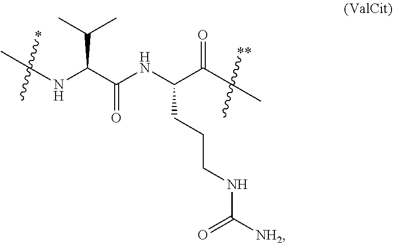

where the * of Lp indicates the attachment point to L₁ and the ** of Lp indicates the attachment point to the —NH— group;
L₃ is a spacer moiety having the structure

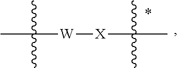

where
   W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

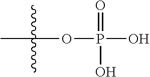 [SMILES: *OP(=O)(O)O]

groups, or a polysarcosine;
and
A is a bond or —OC(═O)—.
Embodiment 47. The linker of any one of Embodiments 32 to 46, having the structure:

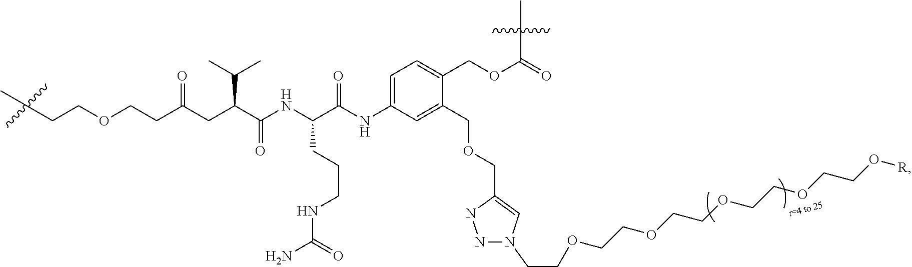

where
R is H, —CH₃ or —CH₂CH₂C(═O)OH.
Embodiment 48. The linker of any one of Embodiments 32 to 46, having the structure:

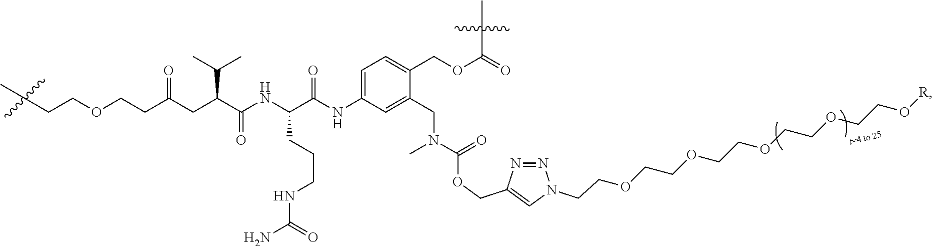

where
R is H, —CH₃ or —CH₂CH₂C(═O)OH.
Embodiment 49. The linker of any one of Embodiments 32 to 46, having the structure:

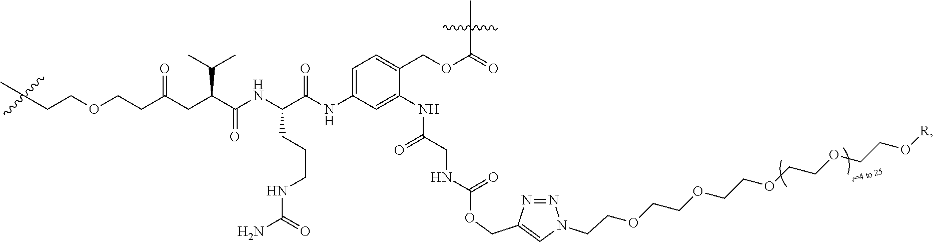

where
R is H, —CH₃ or —CH₂CH₂C(═O)OH.
Embodiment 50. The linker of any one of Embodiments 32 to 46, having the structure:

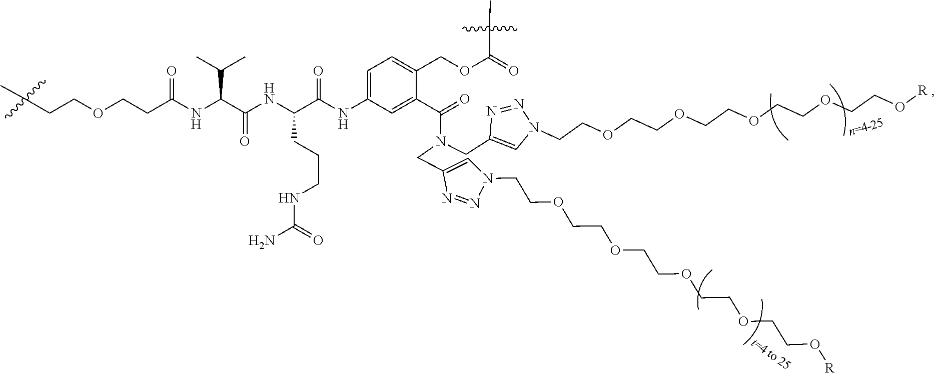

where
each R is independently selected from H, —CH₃ or —CH₂CH₂C(═O)OH.
Embodiment 51. The linker of any one of Embodiments 32 to 46, having the structure:

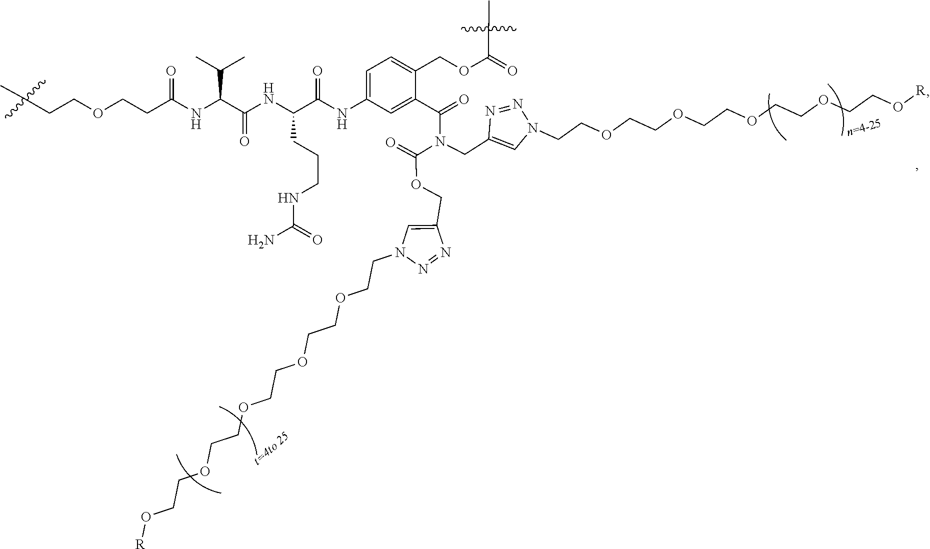

where
each R is independently selected from H, —CH₃ or —CH₂CH₂C(═O)OH.
Embodiment 52. The linker of any one of Embodiments 32 to 46, having the structure:

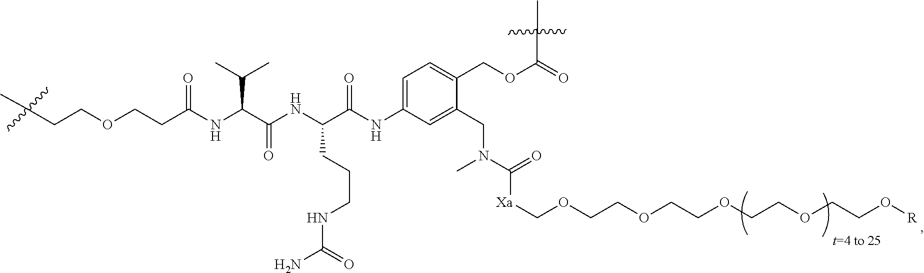

where
Xa is —CH₂—, —OCH₂—, —NHCH₂— or —NRCH₂— and each R independently is H, —CH₃ or —CH₂CH₂C(═O)OH.
Embodiment 53. The linker of any one of Embodiments 32 to 46, having the structure:

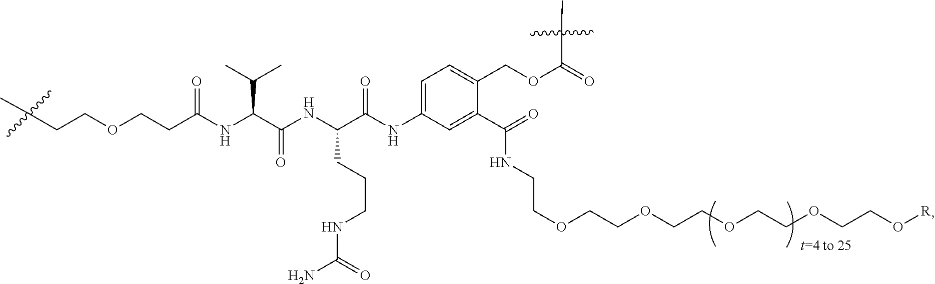

where
R is H, —CH₃ or —CH₂CH₂C(═O)OH.
Embodiment 54 The linker of any one of Embodiments 32 to 46, having the structure:

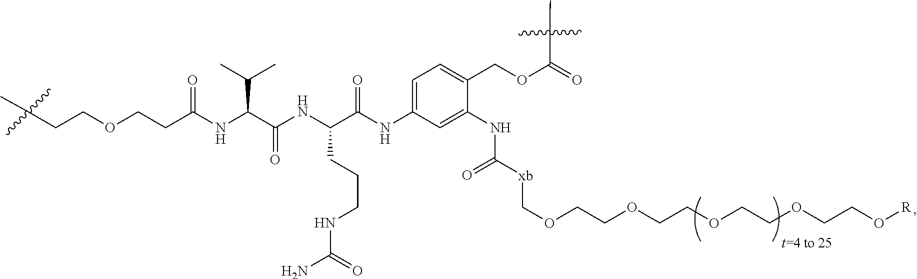

where
Xb is —CH₂—, —OCH₂—, —NHCH₂— or —NRCH₂— and each R independently is H, —CH₃ or —CH₂CH₂C(═O)OH.
Embodiment 55. The linker of any one of Embodiments 32 to 46, having the structure:

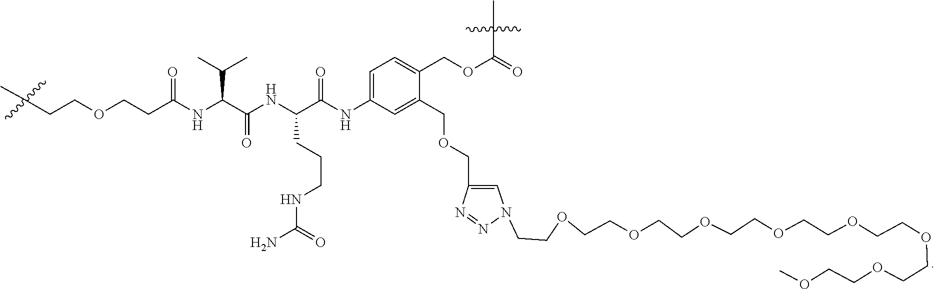 [SMILES: *CCOCCC(=O)N[C@H](C(=O)N[C@@H](CCCNC(N)=O)C(=O)Nc1ccc(COC(*)=O)c(COCc2cn(CCOCCOCCOCCOCCOCCOCCOCCOC)nn2)c1)C(C)C]

Embodiment 56. The linker of any one of Embodiments 32 to 46, having the structure:

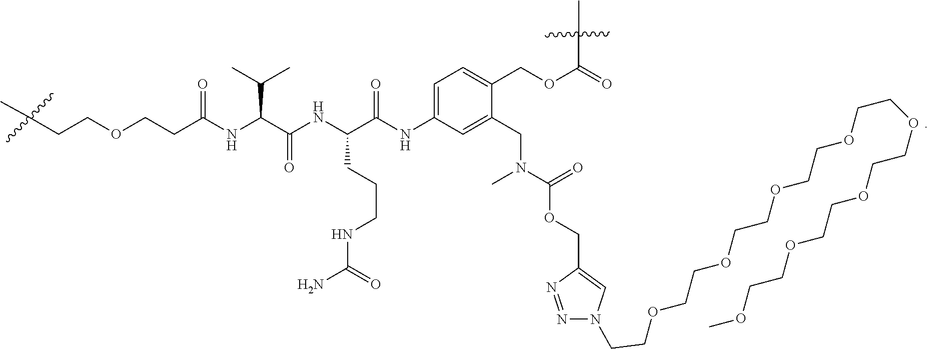 [SMILES: *CCOCCC(=O)N[C@H](C(=O)N[C@@H](CCCNC(N)=O)C(=O)Nc1ccc(COC(*)=O)c(CN(C)C(=O)OCc2cn(CCOCCOCCOCCOCCOCCOCCOCCOC)nn2)c1)C(C)C]

Embodiment 57. The linker of any one of Embodiments 32 to 46, having the structure:

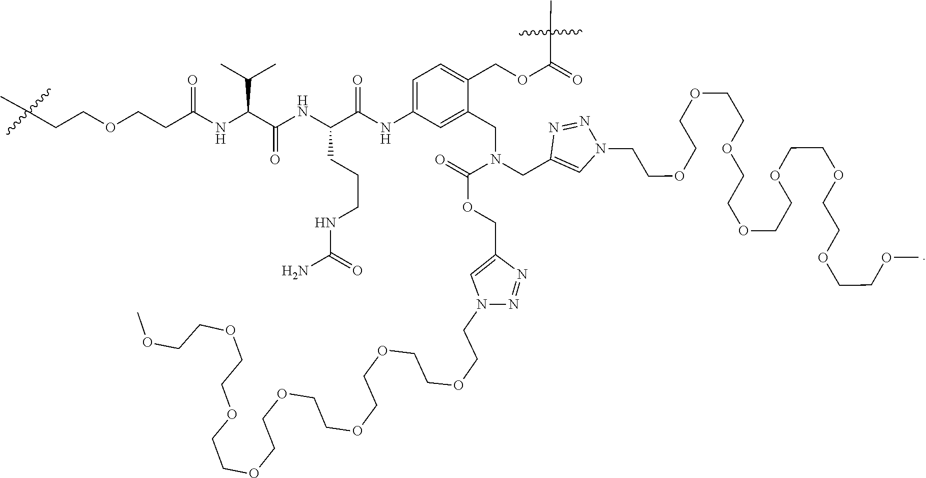 [SMILES: *CCOCCC(=O)N[C@H](C(=O)N[C@@H](CCCNC(N)=O)C(=O)Nc1ccc(COC(*)=O)c(CN(Cc2cn(CCOCCOCCOCCOCCOCCOCCOCCOC)nn2)C(=O)OCc2cn(CCOCCOCCOCCOCCOCCOCCOCCOC)nn2)c1)C(C)C]

Embodiment 58. The linker of any one of Embodiments 32 to 46, having the structure:

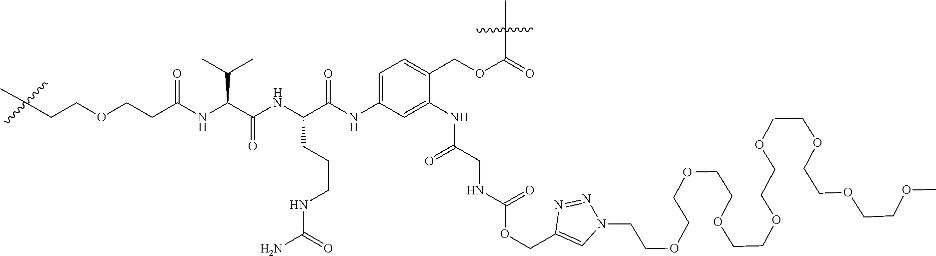 [SMILES: *CCOCCC(=O)N[C@H](C(=O)N[C@@H](CCCNC(N)=O)C(=O)Nc1ccc(COC(*)=O)c(NC(=O)CNC(=O)OCc2cn(CCOCCOCCOCCOCCOCCOCCOCCOC)nn2)c1)C(C)C]

Embodiment 59. The linker of any one of Embodiments 32 to 46, having the structure:

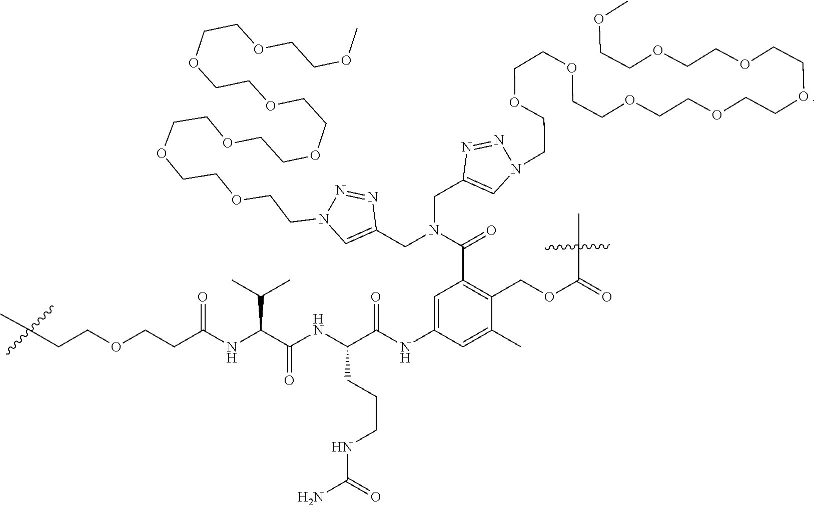 [SMILES: *CCOCCC(=O)N[C@H](C(=O)N[C@@H](CCCNC(N)=O)C(=O)Nc1cc(C)c(COC(*)=O)c(C(=O)N(Cc2cn(CCOCCOCCOCCOCCOCCOCCOCCOC)nn2)Cc2cn(CCOCCOCCOCCOCCOCCOCCOCCOC)nn2)c1)C(C)C]

For illustrative purposes, the general reaction schemes depicted herein provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

By way of example, a general synthesis for compounds of Formula (II) is shown below in Scheme 1.

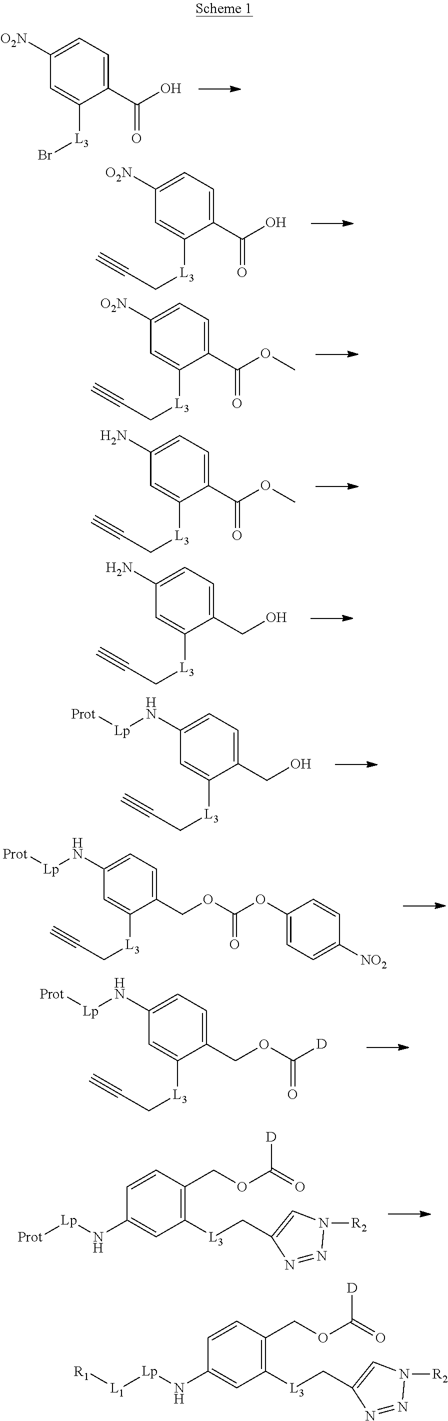

### Antibody Drug Conjugates of the Invention

The present invention provides Antibody Drug Conjugates, also referred to herein as immunoconjugates, which comprise linkers which comprise one or more hydrophilic moieties.

The Antibody Drug Conjugates of the invention have the structure of Formula (III):

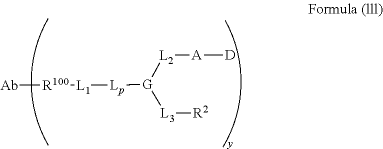

wherein:
Ab is an antibody or fragment thereof;
R¹⁰⁰ is a coupling group;
L₁ is a bridging spacer;
Lp is a bivalent peptide spacer;
G-L₂-A is a self-immolative spacer;
R² is a hydrophilic moiety;
L₂ is a bond, a methylene, a neopentylene or a C₂-C₃alkenylene;
A is a bond, —OC(═O)—*,

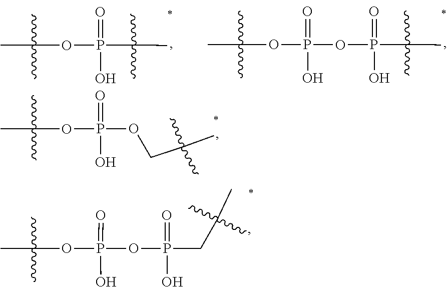

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.

Certain aspects and examples of the Antibody Drug Conjugates of the invention are provided in the following listing of enumerated embodiments. It will be recognized that features specified in each embodiment may be combined with other specified features to provide further embodiments of the present invention.
Embodiment 60. The immunoconjugate of Formula (III) wherein:
   Ab is an antibody or fragment thereof;
   R¹⁰⁰ is a coupling group;
   L₁ is a bridging spacer;
   Lp is a bivalent peptide spacer comprising one to four amino acid residues;
   G-L₂-A is a self-immolative spacer;
   R² is a hydrophilic moiety;
   L₂ is a bond, a methylene, a neopentylene or a C₂-C₃alkenylene;
   A is a bond, —OC(═O)—*,

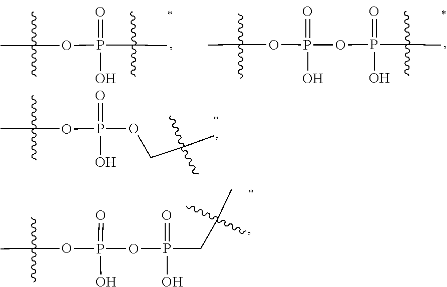

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
Embodiment 61. The immunoconjugate of Formula (III) or Embodiment 60, wherein:
   Ab is an antibody or fragment thereof;
   R¹⁰⁰ is a coupling group;
   L₁ is a bridging spacer;
   Lp is a bivalent peptide spacer comprising one to four amino acid residues;
   the

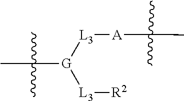

group is selected from:

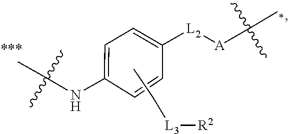

wherein the * of

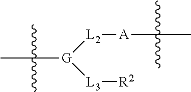

indicates the point of attachment to an N or a O of the Drug moiety, the *** of

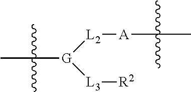

indicates the point of attachment to Lp;
R² is a hydrophilic moiety;
L₂ is a bond, a methylene, a neopentylene or a C₂-C₃alkenylene;
A is a bond, —OC(═O)—*,

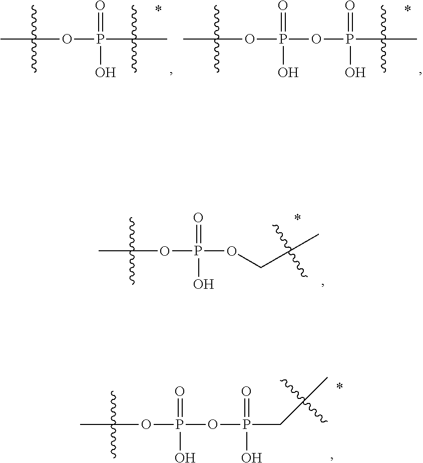 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
Embodiment 62. The immunoconjugate of Formula (III) or any one of Embodiments 60 to 61 having the structure of Formula (IV),

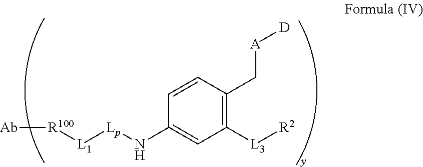

wherein:
Ab is an antibody or fragment thereof;
R¹⁰⁰ is a coupling group;
L₁ is a bridging spacer;
Lp is a bivalent peptide spacer comprising one to four amino acid residues;
R² is a hydrophilic moiety;
A is a bond, —OC(═O)—*,

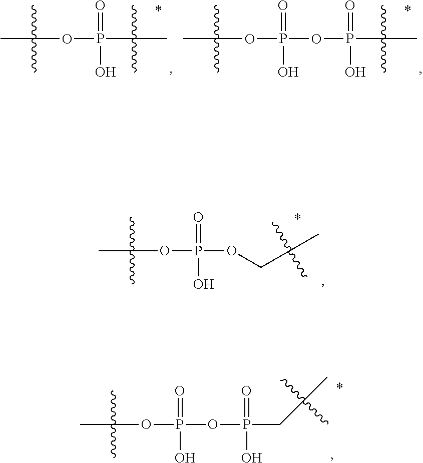 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
Embodiment 63. The immunoconjugate of Formula (III) or any one of Embodiments 60 to 62, wherein:
   Ab is an antibody or fragment thereof;
   R¹⁰⁰ is

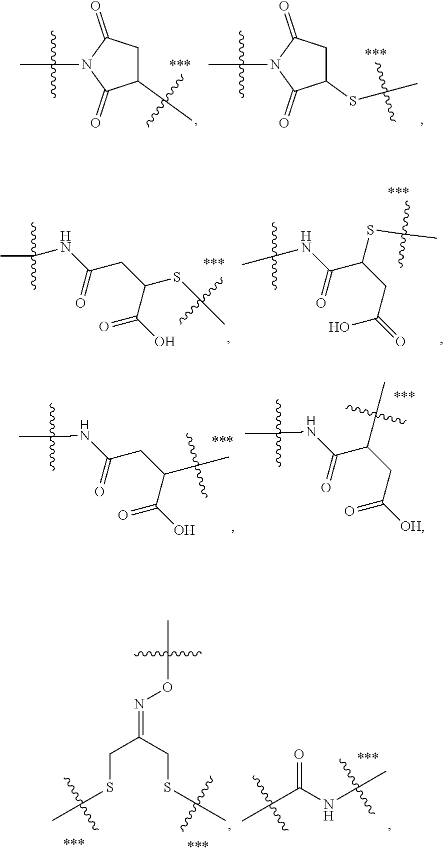

—S—, —C(═O)—, —ON=***, —NHC(═O)CH₂—*** —S(═O)₂CH₂CH₂—***, —(CH₂)₂S(═O)₂CH₂CH₂—***, —NHS(═O)₂CH₂CH₂—***, —NHC(═O)CH₂CH₂—***, —CH₂NHCH₂CH₂—***, —NHCH₂CH₂—***,

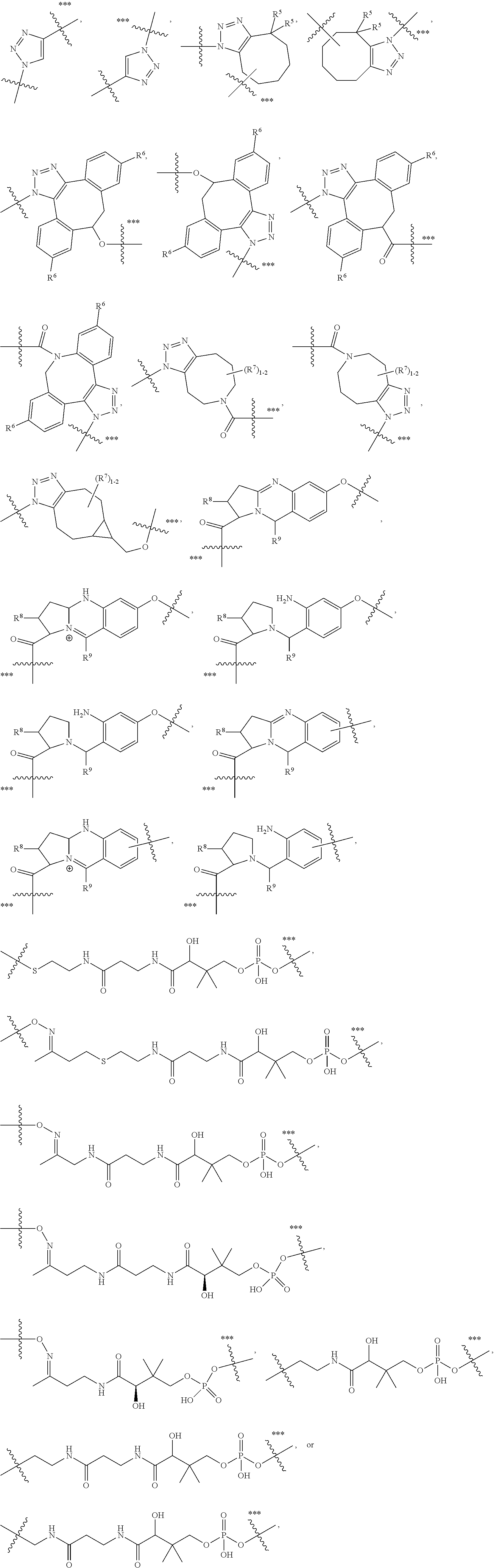

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp, and the ** of L₁ indicates the point of attachment to R¹⁰⁰;
R² is a hydrophilic moiety selected from polyethylene glycol, polyalkylene glycol, a sugar, an oligosaccharide, a polypeptide, C₂-C₆alkyl substituted with 1 to 3

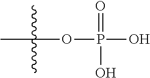 [SMILES: *OP(=O)(O)O]

groups, and a polysarcosine;
each R³ is independently selected from H and C₁-C₆alkyl;
R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —CN, —NO₂ and —OH;
each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
X₁ is

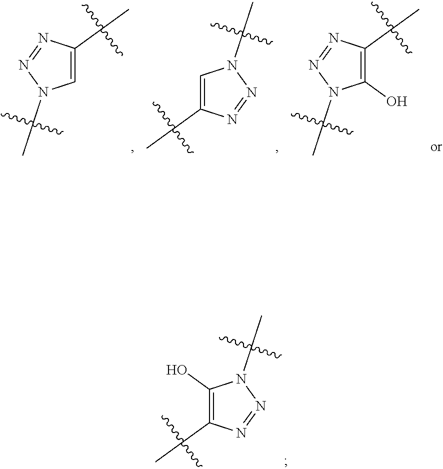 [SMILES: *c1cn(*)nn1 ; *c1cn(*)nn1 ; *c1nnn(*)c1O ; *c1nnn(*)c1O]

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from valine, citrulline, lysine, isoleucine, phenylalanine, methionine, asparagine, proline, alanine, leucine, tryptophan, and tyrosine;
A is a bond, —OC(═O)—*,

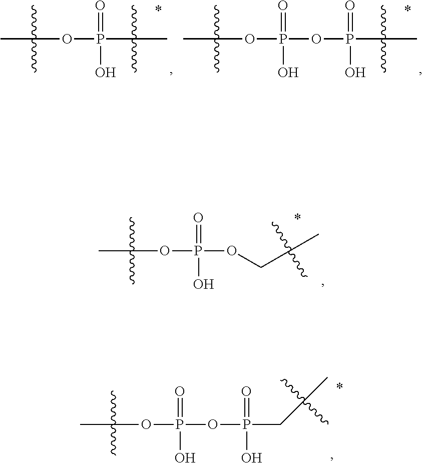 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety having the structure

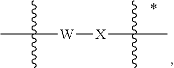

where
   (i) W is —CH₂O—**, —CH₂N(R^{b})C(═O)O—**, —NHC(═O)C(R^{b})₂NHC(═O)O—**, —NHC(═O)C(R^{b})₂NH—**, NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—**, —NH—, or —CH₂N(R^{b})C(═O)CH₂—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²; or
   (ii) W is —CH₂O—**, —CH₂N(R^{b})C(═O)O—**, —NHC(═O)C(R^{b})₂NHC(═O)O—**, —NHC(═O)C(R^{b})₂NH—**, NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is ***—CH₂-triazolyl-C₁₋₄ alkylene-OC(O)NHS(O)₂NH—*, ***—C₄₋₆ cycloalkylene-OC(O)NHS(O)₂NH—*, ***—(CH₂CH₂O),—C(O)NHS(O)₂NH—*, ***—(CH₂CH₂O)ₙ—C(O)NHS(O)₂NH—(CH₂CH₂O)ₙ—*, or ***—CH₂-triazolyl-C₁₋₄ alkylene-OC(O)NHS(O)₂NH—(CH₂CH₂O)ₙ—*, wherein each n independently is 1, 2, or 3, the *** of X indicates the point of attachment to W and the * of X indicates the point of attachment to R²;
   and
   the * of L₃ indicates the point of attachment to R²;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
Embodiment 64. The immunoconjugate of Formula (III) or any one of Embodiments 60 to 63, wherein:
   Ab is an antibody or fragment thereof;
   R¹⁰⁰ is

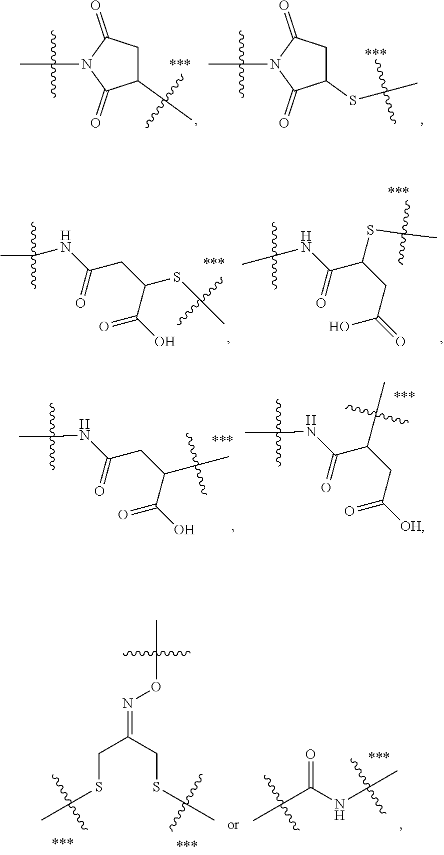

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

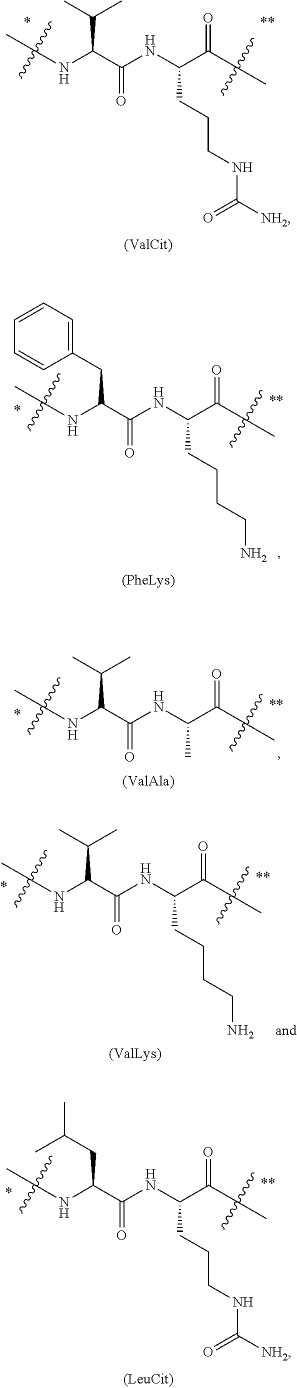

where the * of Lp indicates the attachment point to L₁ and the ** of Lp indicates the attachment point to the —NH— group of G;
L₃ is a spacer moiety having the structure

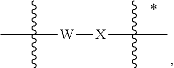

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

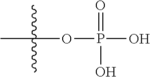 [SMILES: *OP(=O)(O)O]

groups, and a polysarcosine;
A is a bond, —OC(═O)—*,

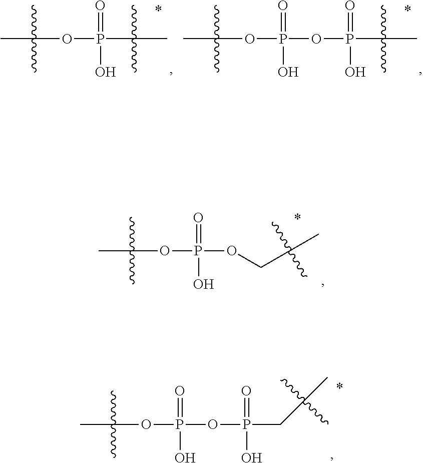 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
Embodiment 65. The immunoconjugate of Formula (III) or any one of Embodiments 60 to 64, wherein:
   Ab is an antibody or fragment thereof;
   R¹⁰⁰ is

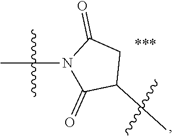 [SMILES: *C1CC(=O)N(*)C1=O]

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

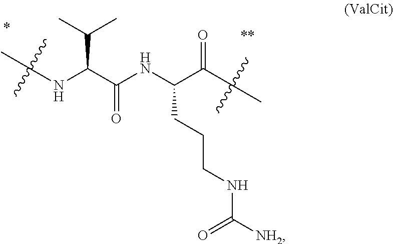 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

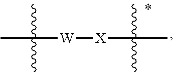

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

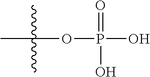 [SMILES: *OP(=O)(O)O]

groups, and a polysarcosine;
A is a bond, —OC(═O)—*,

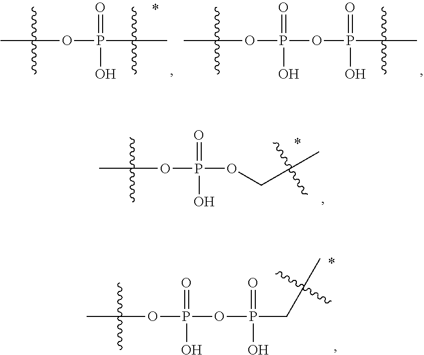 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
Embodiment 66. The immunoconjugate of Formula (III) or any one of Embodiments 60 to 65, wherein:
   Ab is an antibody or fragment thereof;
   R¹⁰⁰ is

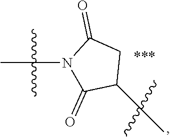 [SMILES: *C1CC(=O)N(*)C1=O]

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

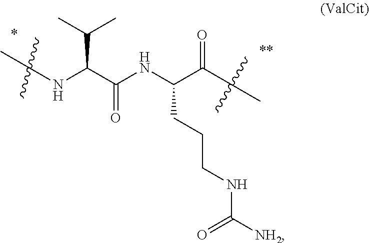 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

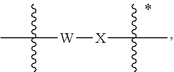

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

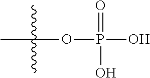 [SMILES: *OP(=O)(O)O]

groups, and a polysarcosine;
A is a bond or —OC(═O)*, in which * indicates the attachment point to D;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
Embodiment 67. The immunoconjugate of Formula (III) or any one of Embodiments 60 to 66, wherein:
   Ab is an antibody or fragment thereof;
   R¹⁰⁰

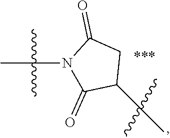 [SMILES: *C1CC(=O)N(*)C1=O]

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ, where the * of L₁ indicates the point of attachment to Lp and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;

Lp is a bivalent peptide spacer selected from

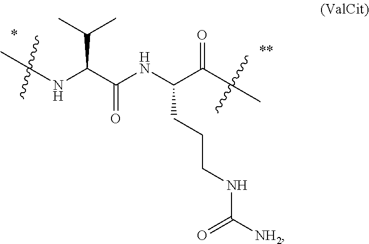 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

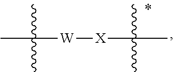

where
   W is —CH₂O—**, —CH₂N(R )C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

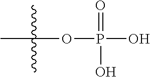 [SMILES: *OP(=O)(O)O]

groups, and a polysarcosine;
A is a bond or —OC(═O)*, in which * indicates the attachment point to D;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
Embodiment 68. The immunoconjugate of Formula (III) or any one of Embodiments 60 to 62, wherein R¹⁰⁰ is a coupling group.
Embodiment 69. The immunoconjugate of Formula (III) or any one of Embodiments 60 to 63, wherein
   R¹⁰⁰ is

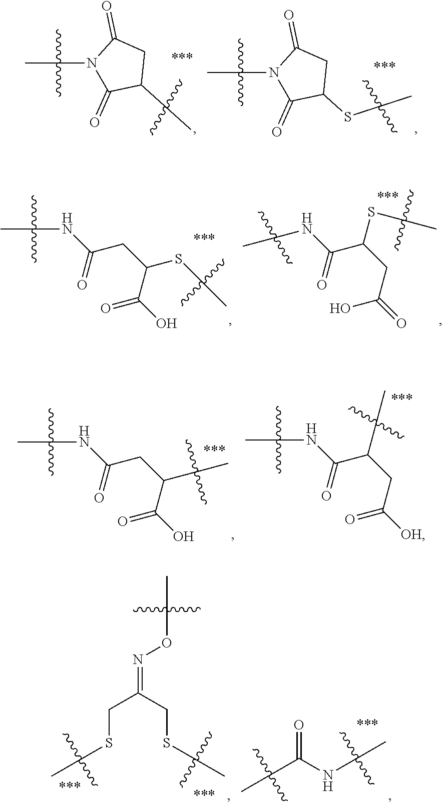

—S—, —C(═O)—, —ON=*** —NHC(═O)CH₂—*** —S(═O)₂CH₂CH₂—***, —(CH₂)₂S(═O)₂CH₂CH₂—***, —NHS(═O)₂CH₂CH₂_***, —NHC(═O)CH₂CH₂—*** —CH₂NHCH₂CH₂—***, —NHCH₂CH₂—***,

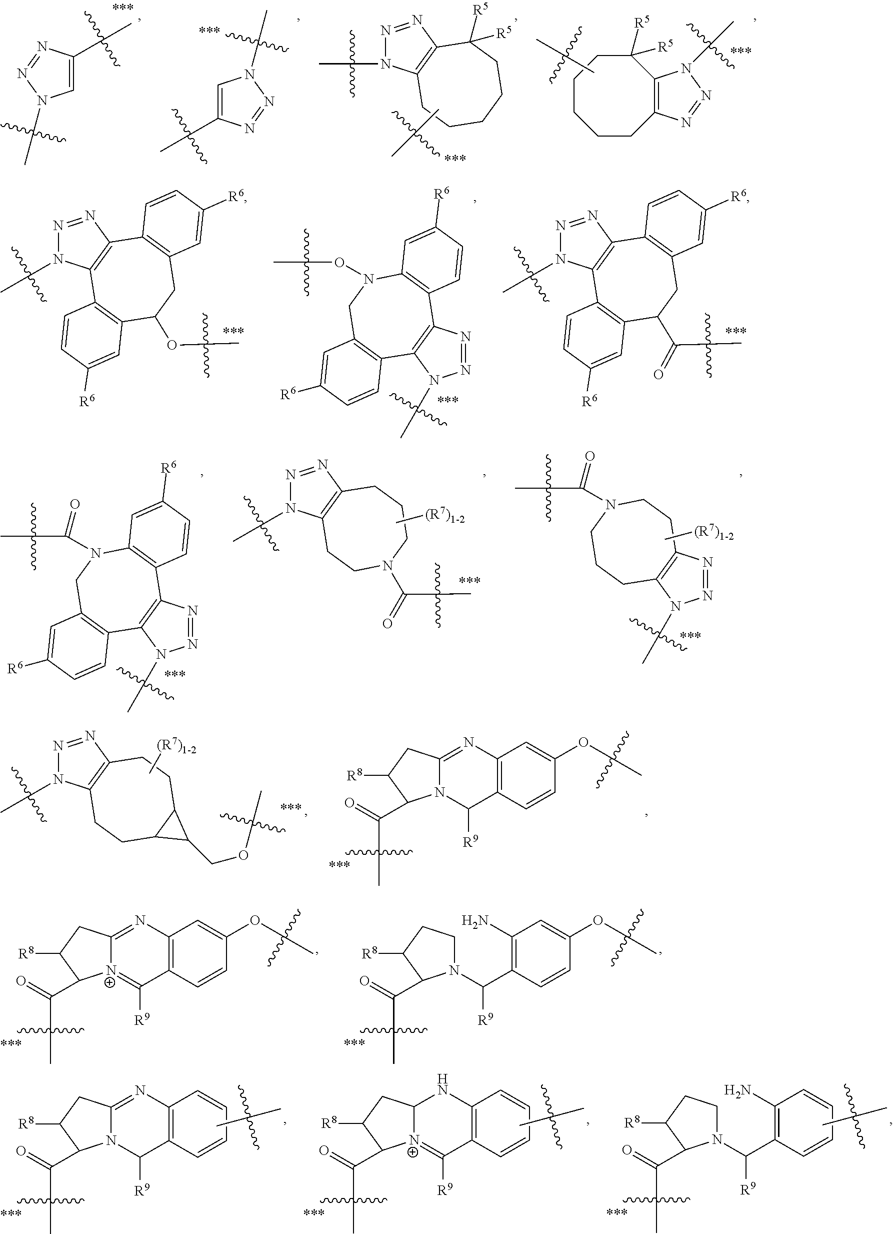

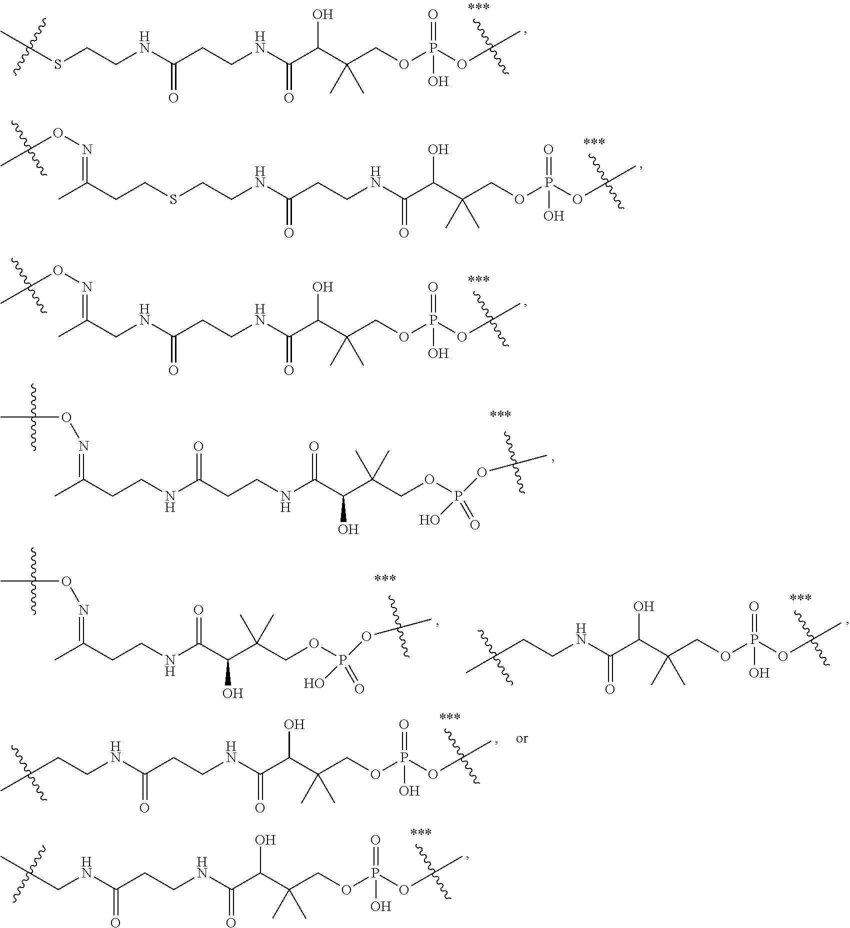

where the *** of R¹⁰⁰ indicates the point of attachment to Ab.
Embodiment 70. The immunoconjugate of Formula (III) or any one of Embodiments 60 to 63, wherein
   R¹⁰⁰ is

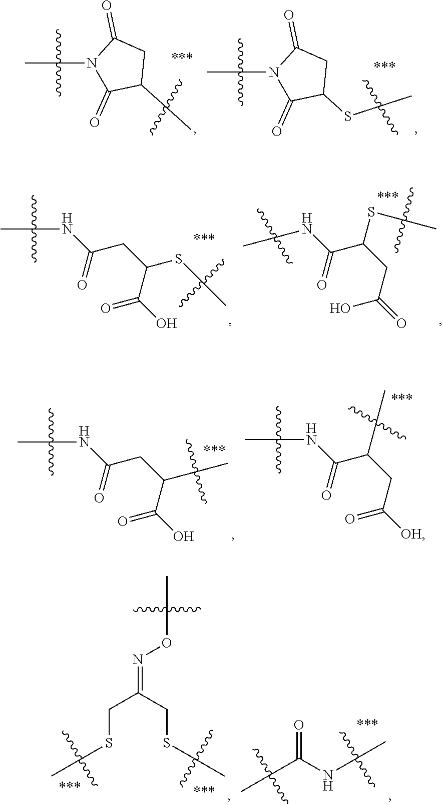

—S—, —C(═O)—, —ON=***, —NHC(═O)CH₂—***, —S(═O)₂CH₂CH₂—***, —(CH₂)₂S(═O)₂CH₂CH₂—***, —NHS(═O)₂CH₂CH₂—***, —NHC(═O)CH₂CH₂—***, —CH₂NHCH₂CH₂—***, —NHCH₂CH₂—***,

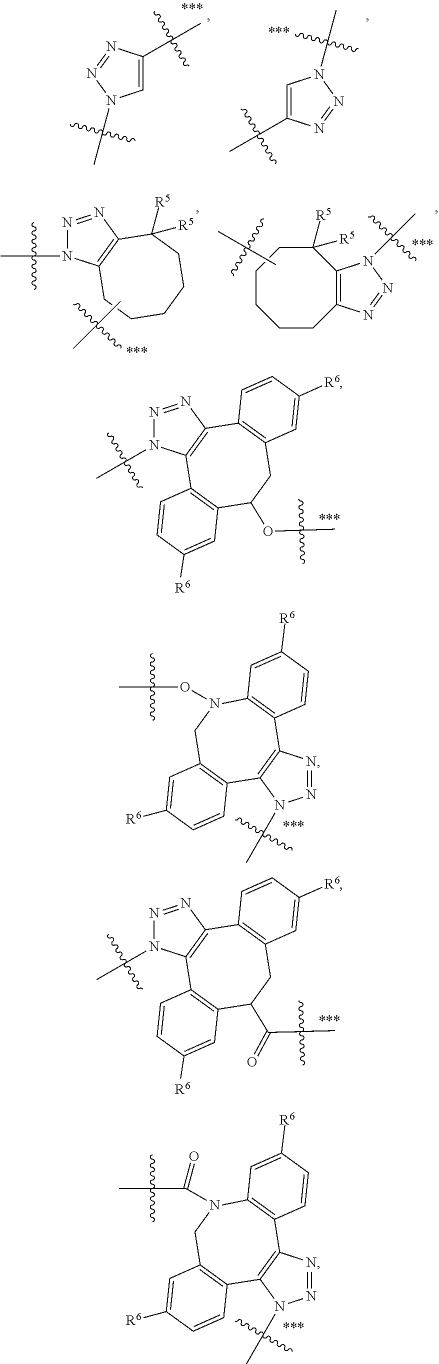

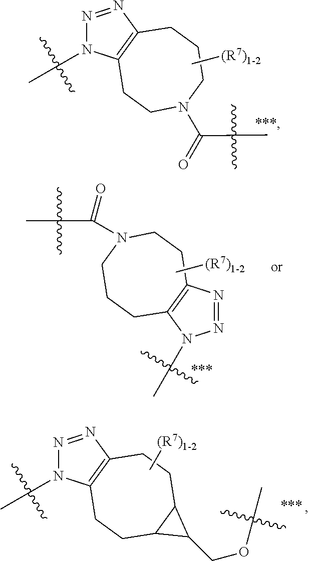

where the *** of R¹⁰⁰ indicates the point of attachment to Ab.
Embodiment 71. The immunoconjugate of Formula (III) or any one of Embodiments 60 to 63, wherein
   R¹⁰⁰ is

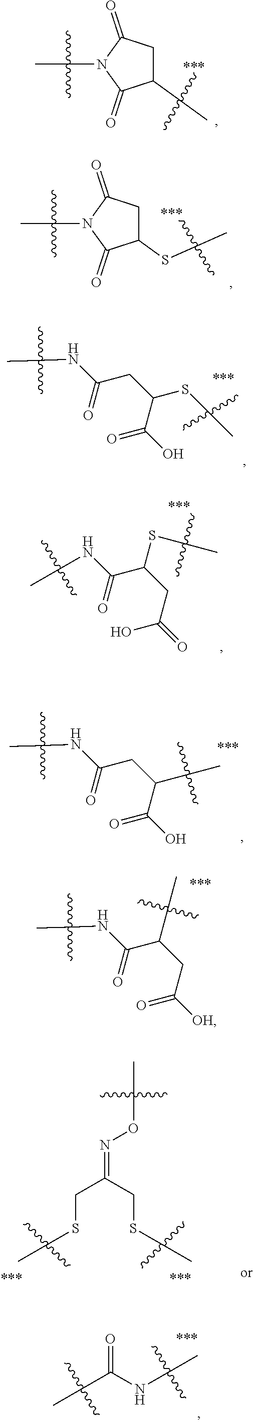

where the *** of R¹⁰⁰ indicates the point of attachment to Ab.
Embodiment 72. The immunoconjugate of Formula (III) or any one of Embodiments 60 to 63, wherein
   R¹⁰⁰ is

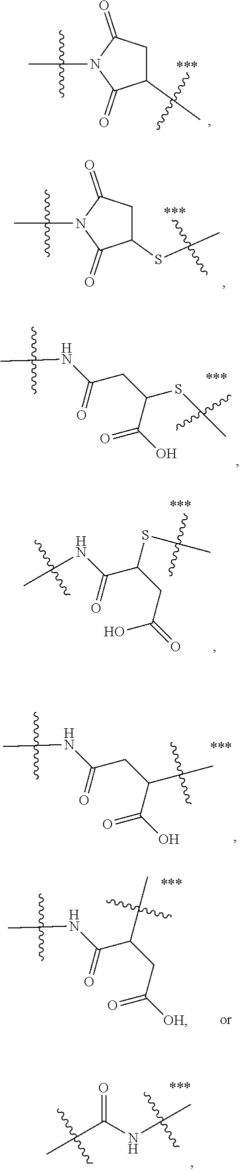

where the *** of R¹⁰⁰ indicates the point of attachment to Ab.
Embodiment 73. The immunoconjugate of Formula (III) or any one of Embodiments 60 to 72 having the structure:

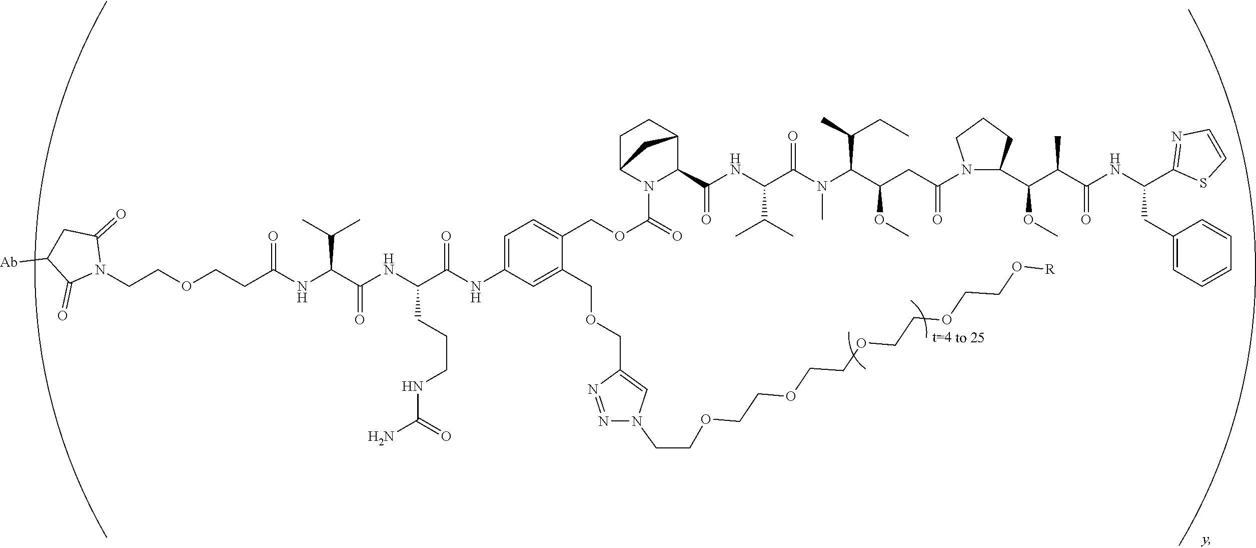

where
R is H, —CH₃ or —CH₂CH₂C(═O)OH and y is 1, 2, 3, 4, 5, 6, 7, 8, 9, 10, 11, 12, 13, 14, 15 or 16.
Embodiment 74. The immunoconjugate of Formula (III) or any one of Embodiments 60 to 72 having the structure:

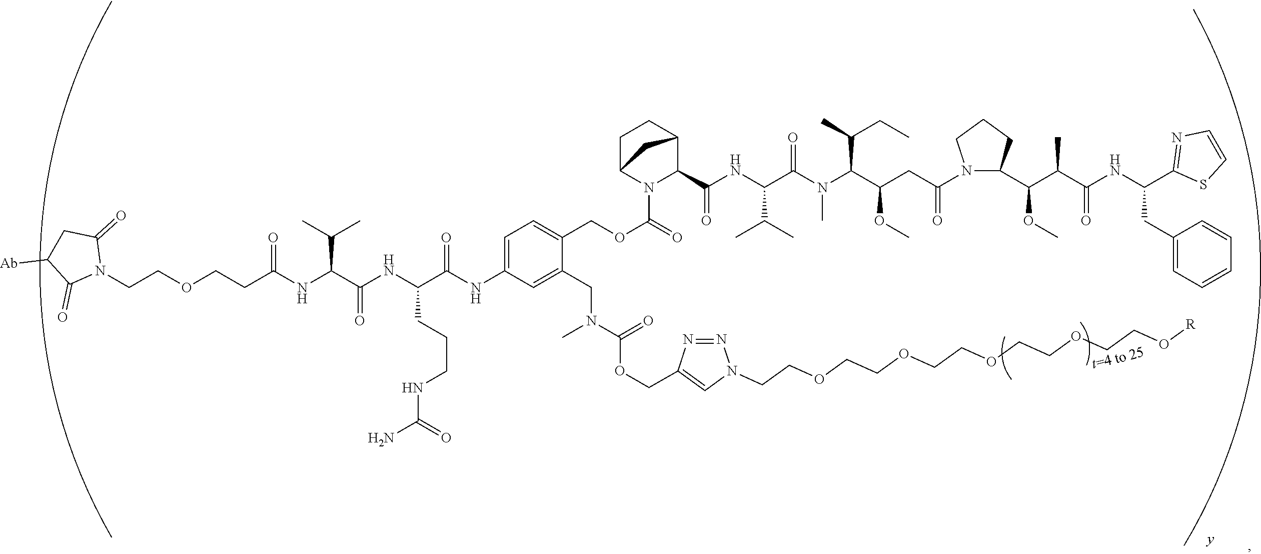

where
R is H, —CH₃ or —CH₂CH₂C(═O)OH and y is 1, 2, 3, 4, 5, 6, 7, 8, 9, 10, 11, 12, 13, 14, 15 or 16.
Embodiment 75. The immunoconjugate of Formula (III) or any one of Embodiments 60 to 72 having the structure:

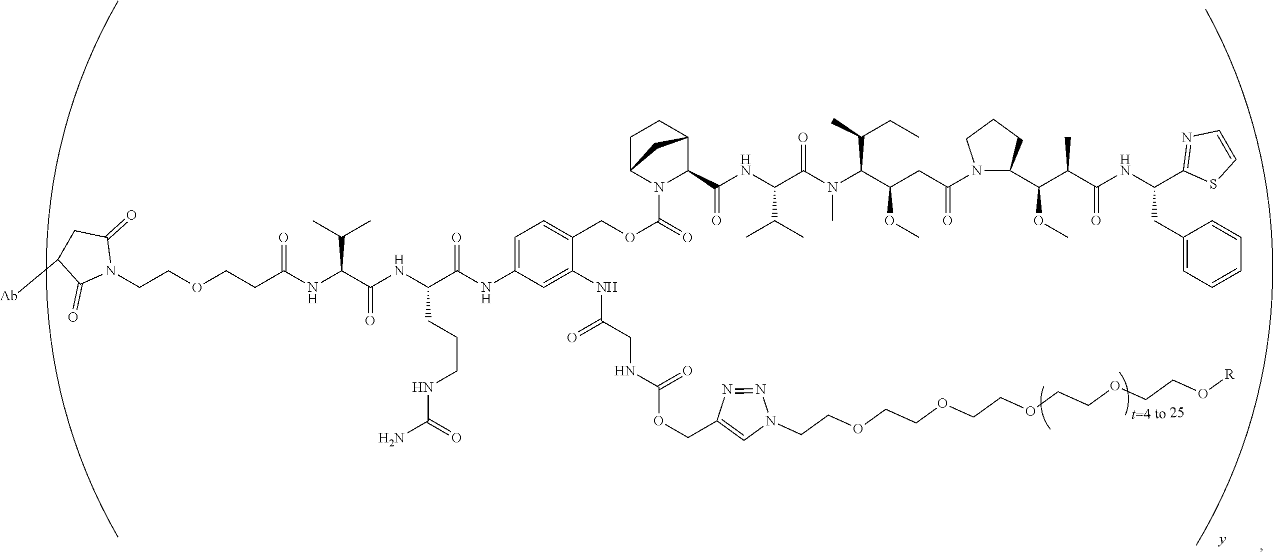

where
R is H, —CH₃ or —CH₂CH₂C(═O)OH and y is 1, 2, 3, 4, 5, 6, 7, 8, 9, 10, 11, 12, 13, 14, 15 or 16.
Embodiment 76. The immunoconjugate of Formula (III) or any one of Embodiments 60 to 72 having the structure:

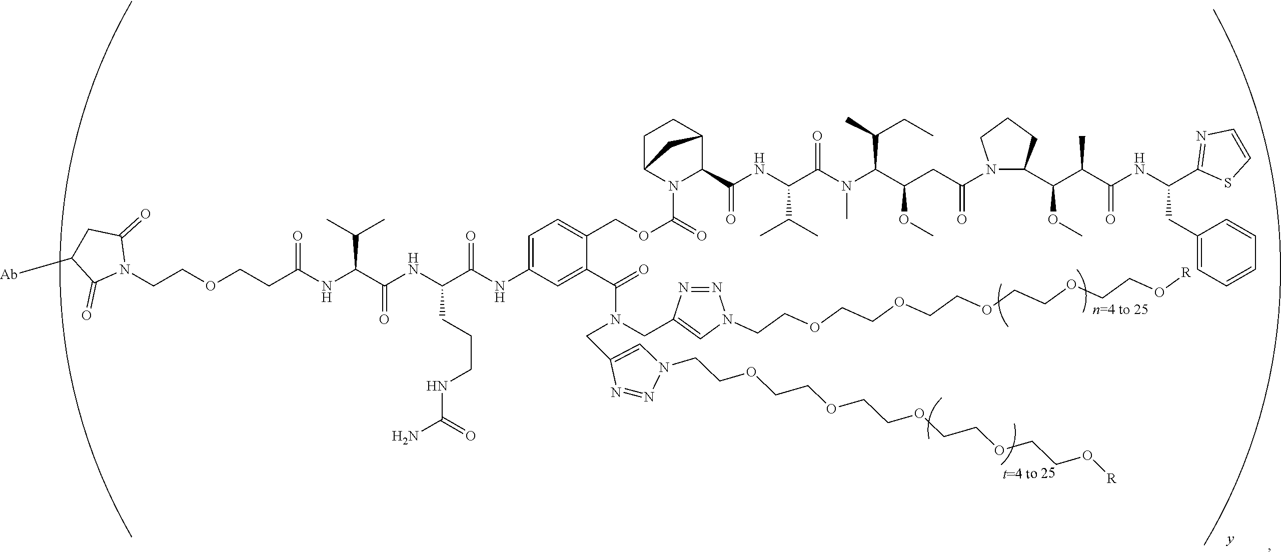

where
each R is independently selected from H, —CH₃ or —CH₂CH₂C(═O)OH and y is 1, 2, 3, 4, 5, 6, 7, 8, 9, 10, 11, 12, 13, 14, 15 or 16.
Embodiment 77. The immunoconjugate of Formula (III) or any one of Embodiments 60 to 72 having the structure:

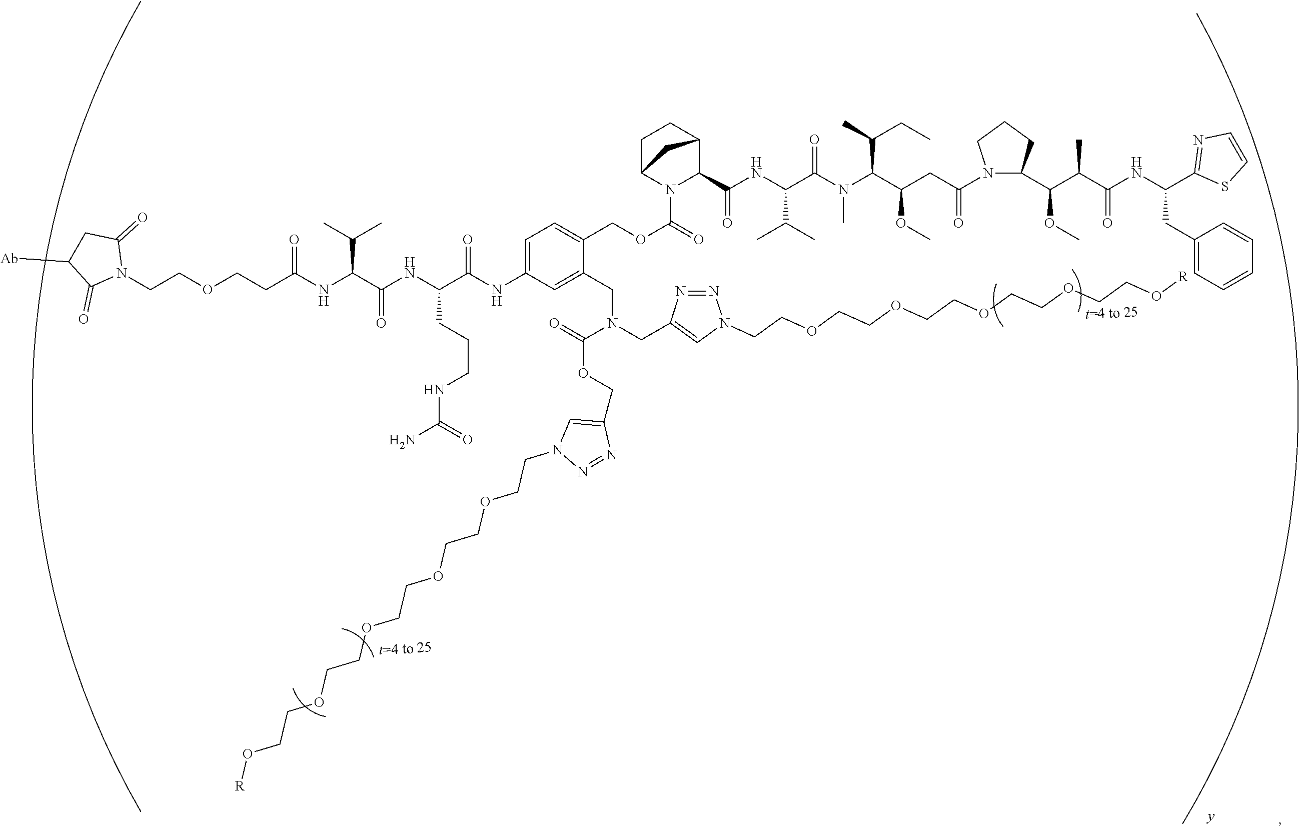

where
each R is independently selected from H, —CH₃ or —CH₂CH₂C(═O)OH and y is 1, 2, 3, 4, 5, 6, 7, 8, 9, 10, 11, 12, 13, 14, 15 or 16.
Embodiment 78. The immunoconjugate of Formula (III) or any one of Embodiments 60 to 72 having the structure:

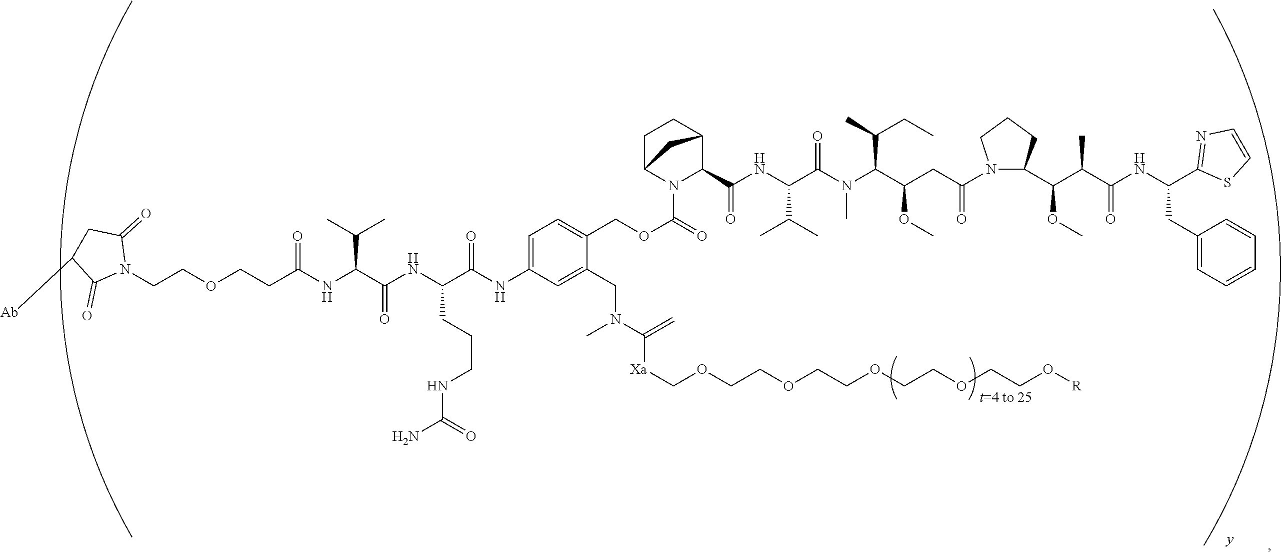

where
Xa is —CH₂—, —OCH₂—, —NHCH₂— or —NRCH₂— and each R independently is H, —CH₃ or —CH₂CH₂C(═O)OH and y is 1, 2, 3, 4, 5, 6, 7, 8, 9, 10, 11, 12, 13, 14, 15 or 16.
Embodiment 79. The immunoconjugate of Formula (III) or any one of Embodiments 60 to 72 having the structure:

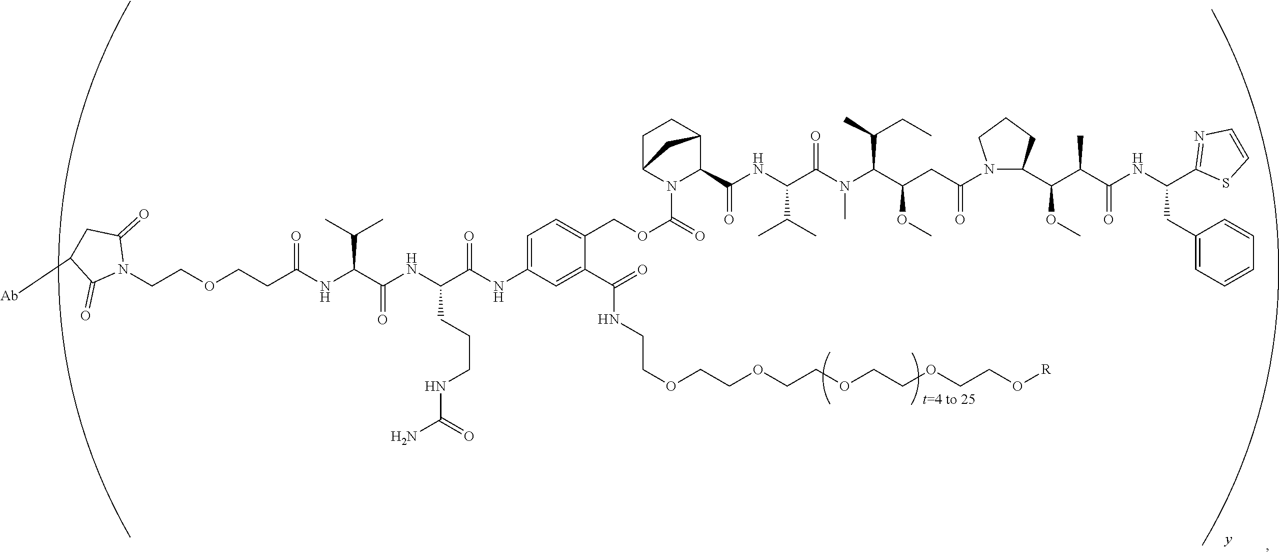

where
R is H, —CH₃ or —CH₂CH₂C(═O)OH and y is 1, 2, 3, 4, 5, 6, 7, 8, 9, 10, 11, 12, 13, 14, 15 or 16.
Embodiment 80. The immunoconjugate of Formula (III) or any one of Embodiments 60 to 72 having the structure:

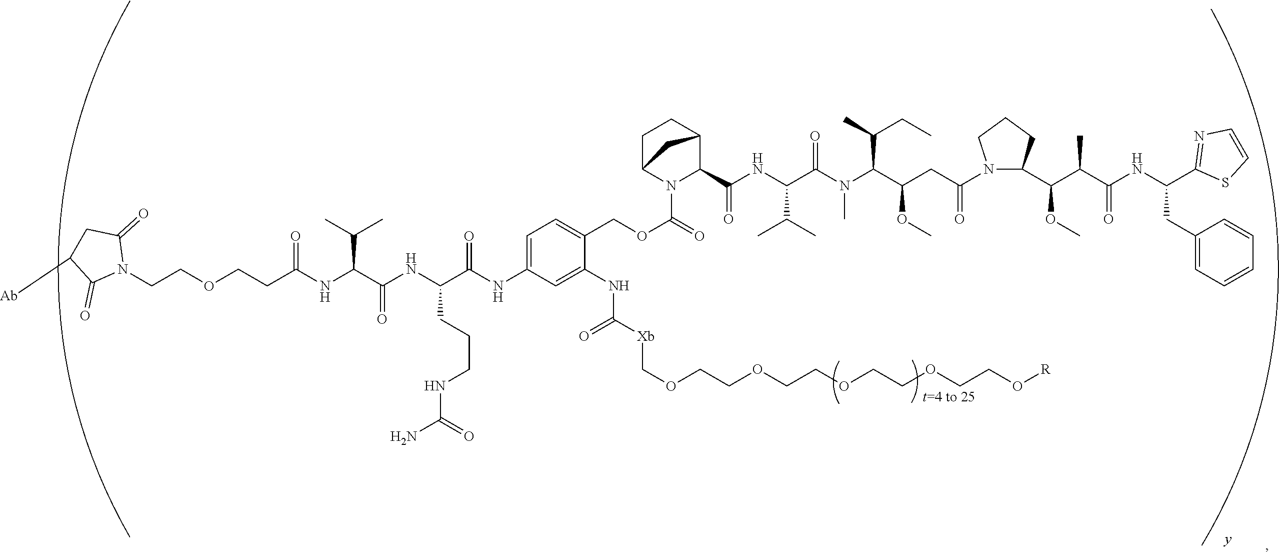

where
Xb is —CH₂—, —OCH₂—, —NHCH₂— or —NRCH₂— and each R independently is H, —CH₃ or —CH₂CH₂C(═O)OH and y is 1, 2, 3, 4, 5, 6, 7, 8, 9, 10, 11, 12, 13, 14, 15 or 16.
Embodiment 81. The immunoconjugate of Formula (III) or any one of Embodiments 60 to 72 having the structure:

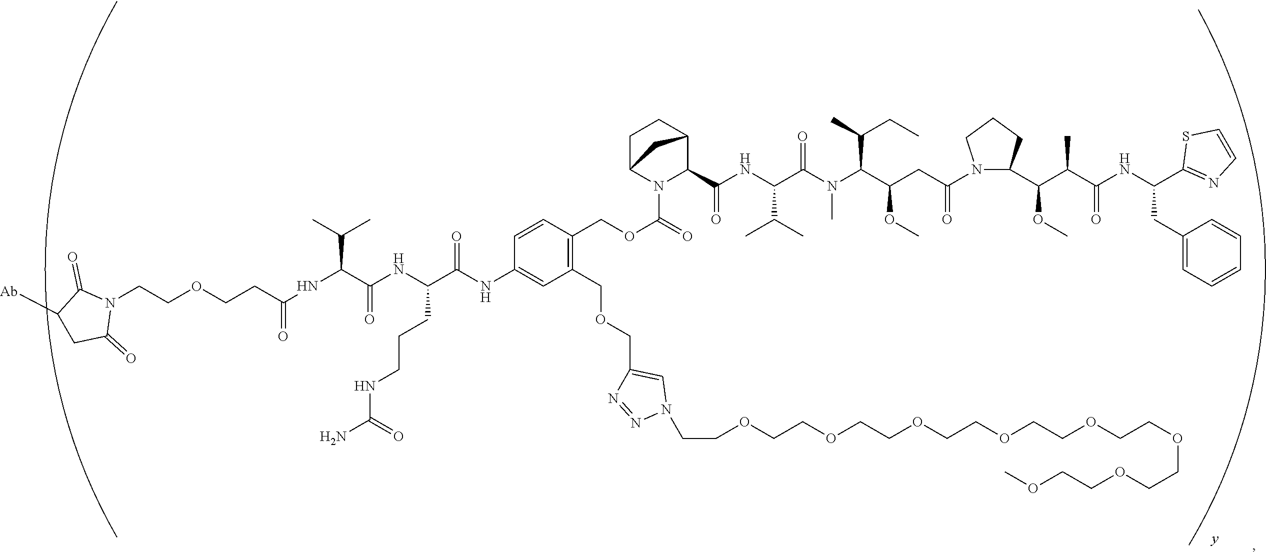

where y is 1, 2, 3, 4, 5, 6, 7, 8, 9, 10, 11, 12, 13, 14, 15 or 16.
Embodiment 82. The immunoconjugate of Formula (III) or any one of Embodiments 60 to 72 having the structure:

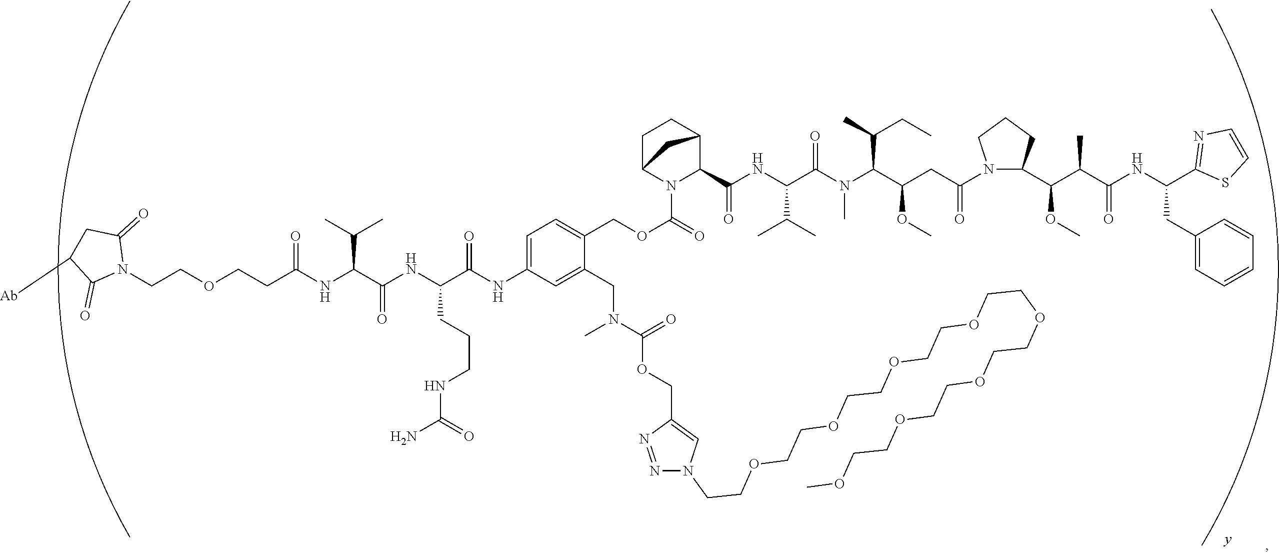

where y is 1, 2, 3, 4, 5, 6, 7, 8, 9, 10, 11, 12, 13, 14, 15 or 16.
Embodiment 83. The immunoconjugate of Formula (III) or any one of Embodiments 60 to 72 having the structure:

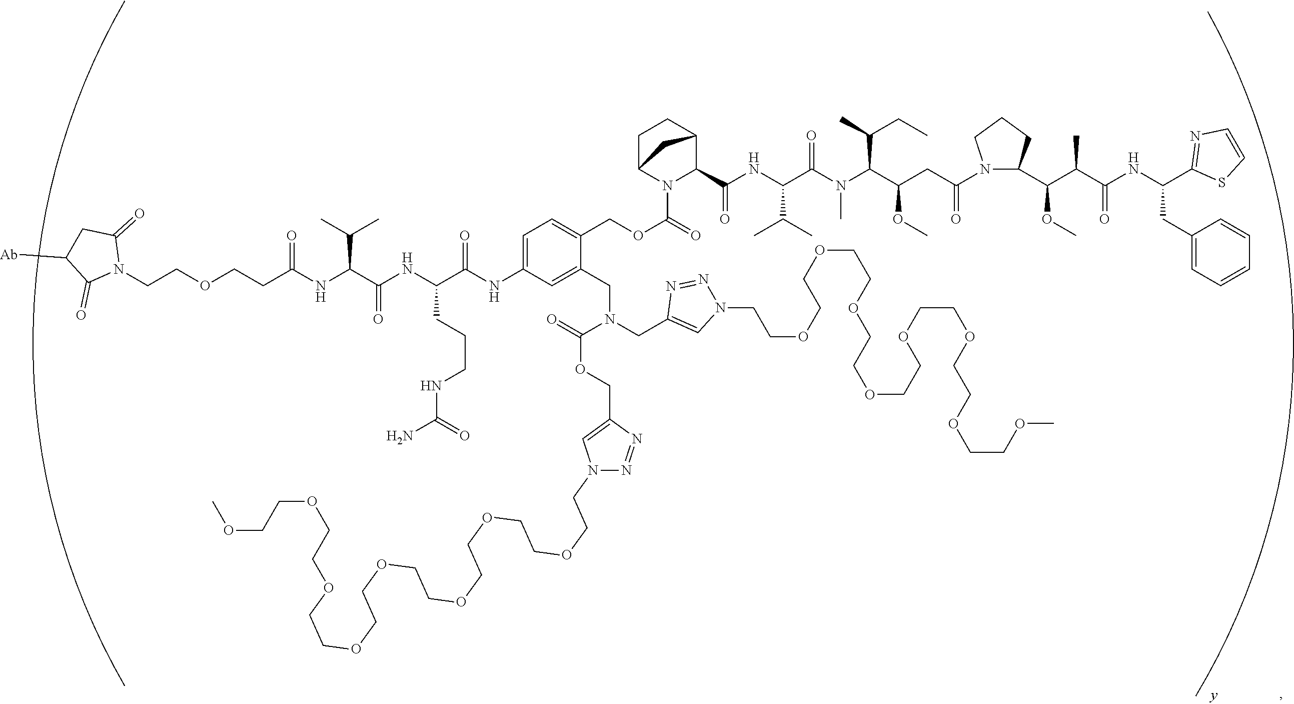

where y is 1, 2, 3, 4, 5, 6, 7, 8, 9, 10, 11, 12, 13, 14, 15 or 16.
Embodiment 84. The immunoconjugate of Formula (III) or any one of Embodiments 60 to 72 having the structure:

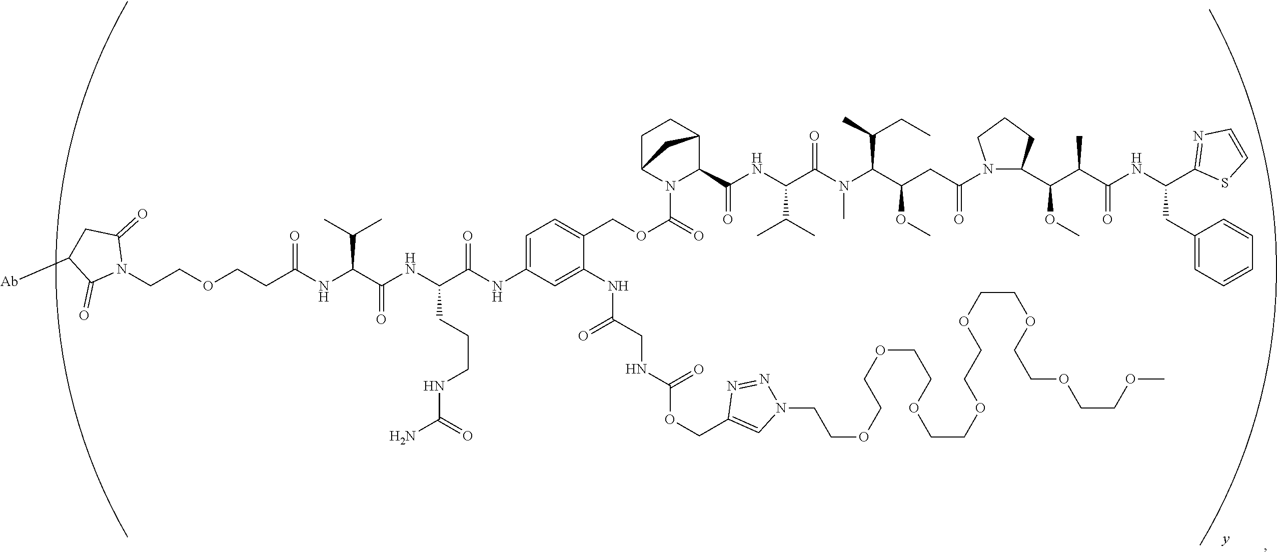

where y is 1, 2, 3, 4, 5, 6, 7, 8, 9, 10, 11, 12, 13, 14, 15 or 16.
Embodiment 85. The immunoconjugate of Formula (III) or any one of Embodiments 60 to 72 having the structure:

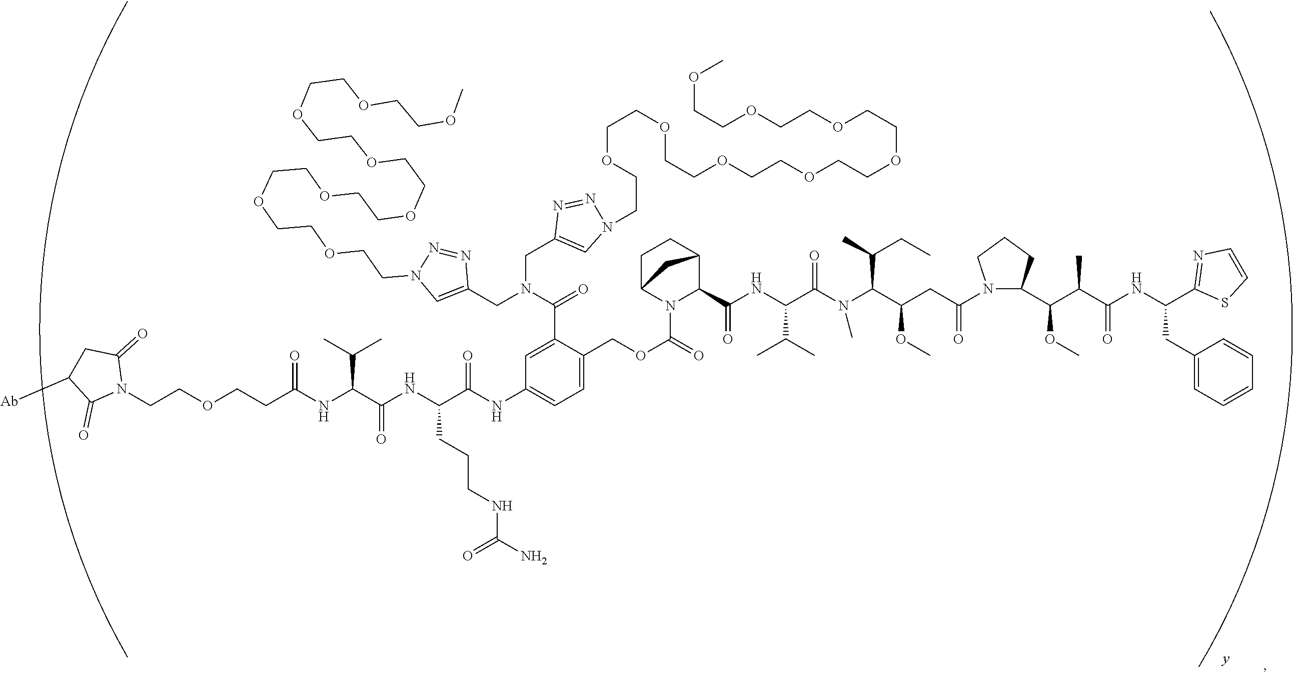

where y is 1, 2, 3, 4, 5, 6, 7, 8, 9, 10, 11, 12, 13, 14, 15 or 16.

Certain aspects and examples of the Linker-Drug groups, the Linkers and the Antibody Drug Conjugates of the invention are provided in the following listing of additional enumerated embodiments. It will be recognized that features specified in each embodiment may be combined with other specified features to provide further embodiments of the present invention.
Embodiment 86. The compound of Formula (I) or any one of Embodiments 1 to 2, or pharmaceutically acceptable salt thereof, the linker of Formula (V or any one of Embodiments 32 to 39, and the immunoconjugate of Formula (III) or any one of Embodiments 60 to 61, wherein:

G is

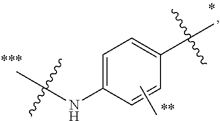

where the * of G indicates the point of attachment to L₂, and the ** of G indicates the point of attachment to L₃ and he *** of G indicates the point of attachment to Lp.
Embodiment 87. The compound of Formula (I) or any one of Embodiments 1 to 2, or pharmaceutically acceptable salt thereof, the linker of Formula (V) or any one of Embodiments 32 to 39, and the immunoconjugate of Formula (III) or any one of Embodiments 60 to 61, wherein:
   G is

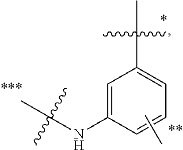

where the * of G indicates the point of attachment to L₂, and the ** of G indicates the point of attachment to L₃ and the *** of G indicates the point of attachment to Lp.
Embodiment 88. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.
Embodiment 89. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)(CH₂)ₘNH(CH₂)ₘ—**; *—C(═O)(CH₂)ₘNH(CH₂)ₙC(═O)—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.
Embodiment 90. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)(CH₂)ₘNH(CH₂)ₘ—**; *—C(═O)(CH₂)ₘNH(CH₂)ₙC(═O)—**; or *—C(═O)(CH₂)ₘNHC(═O)(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.
Embodiment 91. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, wherein:
   L₁ is *—C(═O)(CH₂)ₘO(CH₂)ₘ—**; *—C(═O)((CH₂)ₘO)ₜ(CH₂)ₙ—**; *—C(═O)(CH₂)ₘ—** or *—C(═O)NH((CH₂)ₘO)ₜ(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.
Embodiment 92. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, wherein L₁ is *—C(═O)(CH₂)ₘO(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.
Embodiment 93. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, wherein L₁ is *—C(═O)((CH₂)ₘO)ₜ(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.
Embodiment 94. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, wherein L₁ is *—C(═O)(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.
Embodiment 95. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, wherein L₁ is *—C(═O)NH((CH₂)ₘO)ₜ(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.
Embodiment 96. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein Lp is a bivalent peptide spacer, e.g., an enzymatically cleavable spacer.
Embodiment 97. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein Lp is a bivalent peptide spacer comprising an amino acid residue selected from glycine, valine, citrulline, lysine, isoleucine, phenylalanine, methionine, asparagine, proline, alanine, leucine, tryptophan, and tyrosine.
Embodiment 98. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein Lp is a bivalent peptide spacer comprising one to four amino acid residues, e.g., two to four amino acid residues.
Embodiment 99. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein Lp is a bivalent peptide spacer comprising one to four amino acid residues each independently selected from glycine, valine, citrulline, lysine, isoleucine, phenylalanine, methionine, asparagine, proline, alanine, leucine, tryptophan, and tyrosine.
Embodiment 100. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein:
   Lp is a bivalent peptide spacer selected from

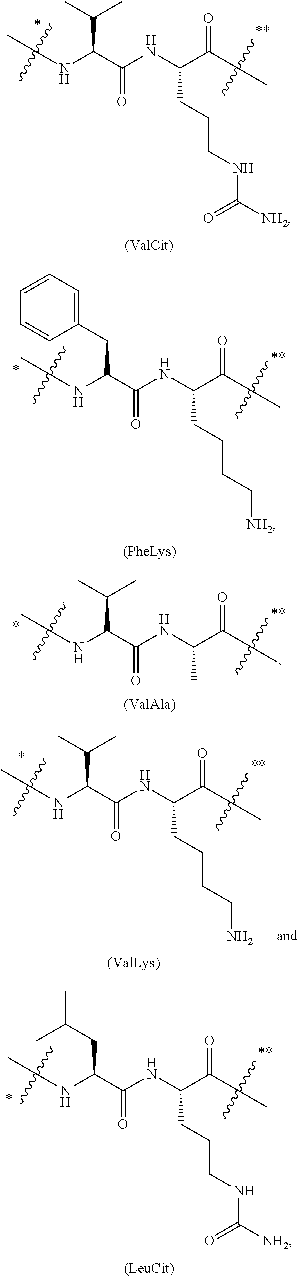

where the * of Lp indicates the attachment point to L₁ and the ** of Lp indicates the attachment point to the —NH— group of Formula (II) or the ** of Lp indicates the attachment point to the G of Formula (I).
Embodiment 101. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein:
   Lp is

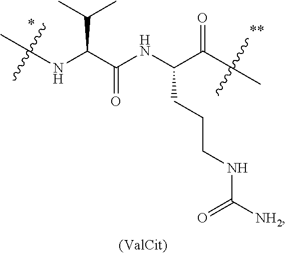 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of Formula (II) or the ** of Lp indicates the attachment point to the G of Formula (I).
Embodiment 102. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein:
   Lp is

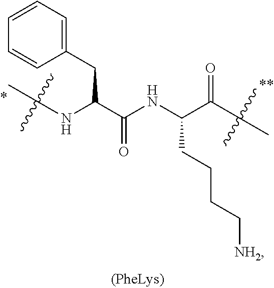

where the * of Lp indicates the attachment point to L₁ and the ** of Lp indicates the attachment point to the —NH— group of Formula (II) or the ** of Lp indicates the attachment point to the G of Formula (I).
Embodiment 103. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein:
   Lp is

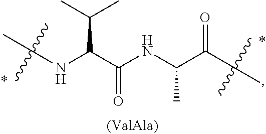

where the * of Lp indicates the attachment point to L₁ and the ** of Lp indicates the attachment point to the —NH— group of Formula (II) or the ** of Lp indicates the attachment point to the G of Formula (I).
Embodiment 104. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein:
   Lp is

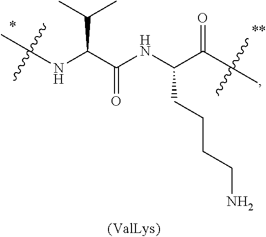

where the * of Lp indicates the attachment point to L₁ and the ** of Lp indicates the attachment point to the —NH— group of Formula (II) or the ** of Lp indicates the attachment point to the G of Formula (I).
Embodiment 105. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 95, wherein:
   Lp is

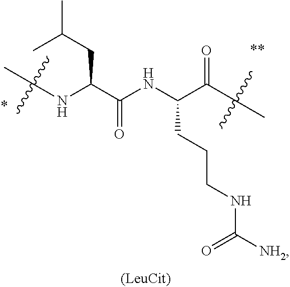 [SMILES: *N[C@@H](CC(C)C)C(=O)N[C@@H](CCCNC(N)=O)C(*)=O]

where the * of Lp indicates the attachment point to L₁ and the ** of Lp indicates the attachment point to —NH— group of Formula (II) or the ** of Lp indicates the attachment point to the G of Formula (I).
Embodiment 106. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 105, wherein L₂ is a bond, a methylene, a neopentylene or a C₂-C₃alkenylene.
Embodiment 107. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 105, wherein L₂ is a bond or a methylene.
Embodiment 108. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 105, wherein L₂ is a bond.
Embodiment 109. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 105, wherein L₂ is a methylene.
Embodiment 110. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 109, wherein:
   A is a bond, —OC(═O)—,

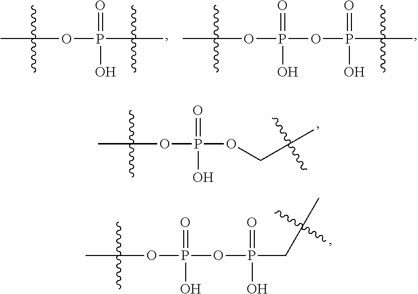

—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl.
Embodiment 111. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 109, wherein A is a bond or —OC(═O).
Embodiment 112. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 109, wherein A is a bond.
Embodiment 113. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 109, wherein A is —OC(═O).
Embodiment 114. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 109, wherein:
   A is

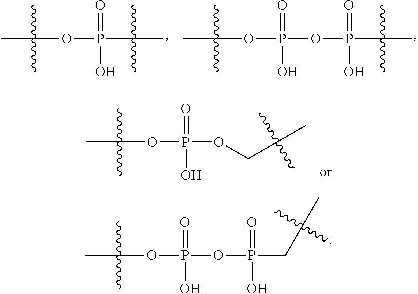

Embodiment 115. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 109, wherein:
   A is —OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl.
Embodiment 116. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 115, wherein L₃ is a spacer moiety.
Embodiment 117. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 115, wherein:
   L₃ is a spacer moiety having the structure

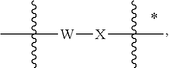

where
   W is —CH₂O—**, —CH₂N(R )C(═O)O—**, —NHC(═O)CH₂NHC(═O)O—**, —NHC(═O)C(R^{b})₂NH—**, NHC(═O)C(R^{b})₂NHC(═O)—**,—CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²; and
the * of L₃ indicates the point of attachment to R².
Embodiment 118. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 115, wherein:
   L₃ is a spacer moiety having the structure

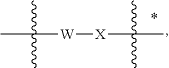

where
   W is —CH₂O—**, —CH₂N(R^{b})C(═O)O—**, —NHC(═O)CH₂NHC(═O)O—**, —NHC(═O)CH₂NH—**, NHC(═O)CH₂NHC(═O)—**,—CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond;
   and
the * of L₃ indicates the point of attachment to R².
Embodiment 119. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 115, wherein:
   L₃ is a spacer moiety having the structure

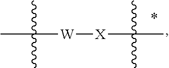

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a triazolyl, wherein the *** of X indicates the point of attachment to W and the * of X indicates the point of attachment to R²;
   and
the * of L₃ indicates the point of attachment to R².
Embodiment 120. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 115, wherein:
   L₃ is a spacer moiety having the structure

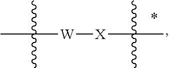

where
   W is —CH₂O—**, —CH₂N(R )C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
the * of L₃ indicates the point of attachment to R².
Embodiment 121. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 115, wherein:
   L₃ is a spacer moiety having the structure

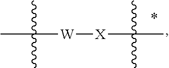

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
the * of L₃ indicates the point of attachment to R².
Embodiment 122. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 115, wherein:
   L₃ is a spacer moiety having the structure

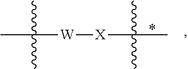

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is a bond;
   and
the * of L₃ indicates the point of attachment to R².
Embodiment 123. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 115, wherein:
   L₃ is a spacer moiety having the structure

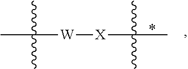

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is a triazolyl, wherein the *** of X indicates the point of attachment to W and the * of X indicates the point of attachment to R²;
   and
the * of L₃ indicates the point of attachment to R².
Embodiment 124. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 115, wherein:
   L₃ is a spacer moiety having the structure

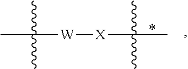

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
the * of L₃ indicates the point of attachment to R².
Embodiment 125. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a hydrophilic moiety.
Embodiment 126. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a hydrophilic moiety selected from polyethylene glycol, polyalkylene glycol, a sugar, an oligosaccharide, a polypeptide, C₂-C₆alkyl substituted with 1 to 3

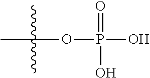 [SMILES: *OP(=O)(O)O]

groups, and a polysarcosine.
Embodiment 127. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a sugar.
Embodiment 128. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is an oligosaccharide.
Embodiment 129. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a polypeptide.
Embodiment 130. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a polyalkylene glycol.
Embodiment 131. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a polyalkylene glycol having the structure —(O(CH₂)ₘ)ₜR′, where R′ is OH, OCH₃ or OCH₂CH₂C(═O)OH, m is 1-10 and t is 4-40.
Embodiment 132. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a polyalkylene glycol having the structure —((CH₂)ₘO)ₜR″—, where R″ is H, CH₃ or CH₂CH₂C(═O)OH, m is 1-10 and t is 4-40.
Embodiment 133. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a polyethylene glycol.
Embodiment 134. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a polyethylene glycol having the structure —(OCH₂CH₂)ₜR′, where R′ is OH, OCH₃ or OCH₂CH₂C(═O)OH and t is 4-40,
Embodiment 135. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein R² is a polyethylene glycol having the structure —(CH₂CH₂O)ₜR″—, where R″ is H, CH₃ or CH₂CH₂C(═O)OH and t is 4-40.
Embodiment 136. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein:
   R² is

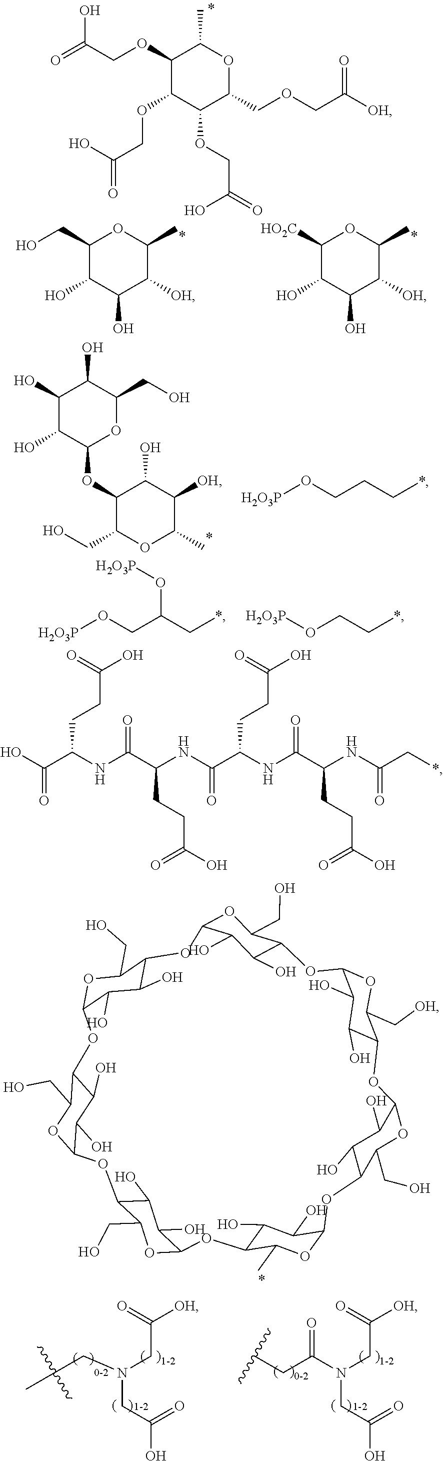

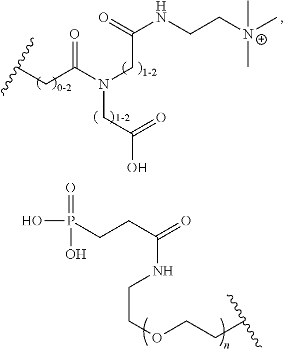

wherein n is an integer between 1 and 6,

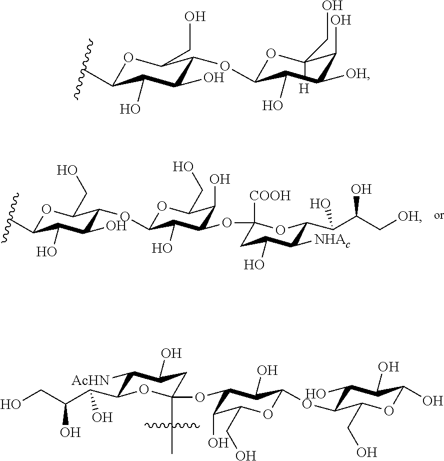

where the * or wavy line of R² indicates the point of attachment to X or L₃.
Embodiment 137. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein:
   R² is

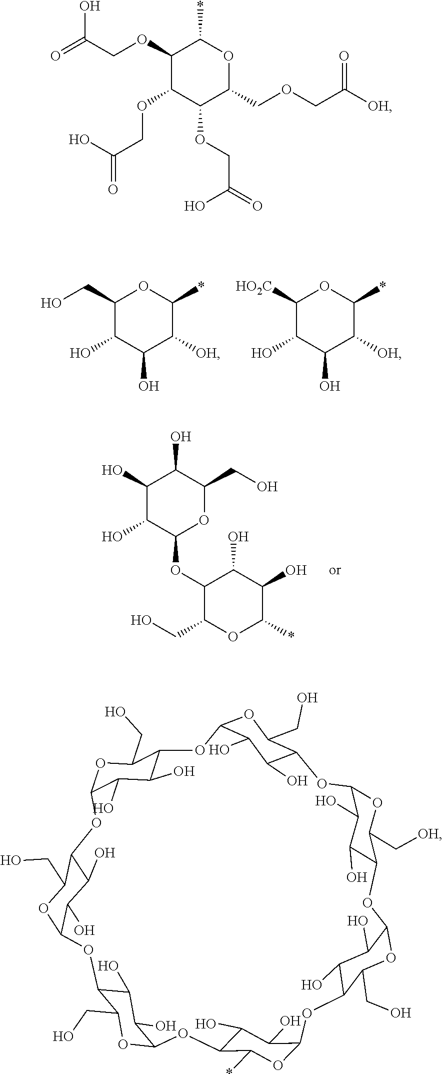 [SMILES: *[C@@H]1O[C@H](COCC(=O)O)[C@H](OCC(=O)O)[C@H](OCC(=O)O)[C@H]1OCC(=O)O ; *[C@@H]1O[C@H](CO)[C@@H](O)[C@H](O)[C@H]1O ; *[C@@H]1O[C@H](C(=O)O)[C@@H](O)[C@H](O)[C@H]1O ; *[C@@H]1O[C@H](CO)C(O[C@@H]2O[C@H](CO)[C@H](O)[C@H](O)[C@H]2O)[C@H](O)[C@H]1O ; *C1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

where the * of R² indicates the point of attachment to X or L₃.
Embodiment 138. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein:
   R² is

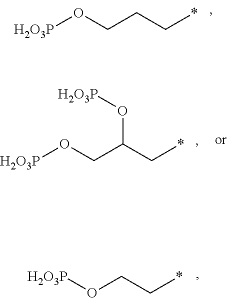

where the * of R² indicates the point of attachment to X or L₃.
Embodiment 139. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 124, wherein:
   R² is

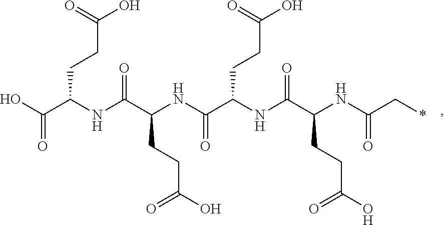 [SMILES: *CC(=O)N[C@@H](CCC(=O)O)C(=O)N[C@@H](CCC(=O)O)C(=O)N[C@@H](CCC(=O)O)C(=O)N[C@@H](CCC(=O)O)C(=O)O]

where the * of R² indicates the point of attachment to X or L₃.
Embodiment 140. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 139, wherein each R³ is independently selected from H and C₁-C₆alkyl.
Embodiment 141. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 139, wherein each R³ is H.
Embodiment 142. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 139, wherein each R³ is independently selected from C₁-C₆alkyl.
Embodiment 143. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 142, wherein:
   X₁ is

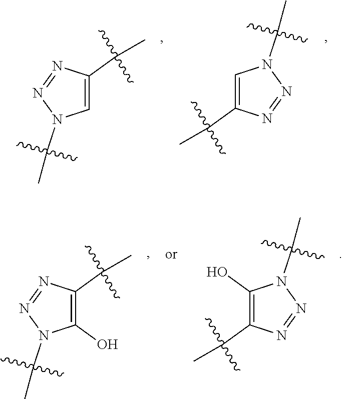 [SMILES: *c1cn(*)nn1 ; *c1cn(*)nn1 ; *c1nnn(*)c1O ; *c1nnn(*)c1O]

Embodiment 144. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 142, wherein:
   X₁ is

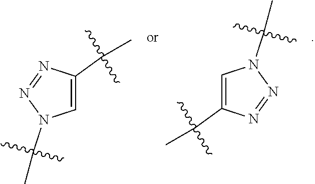 [SMILES: *c1cn(*)nn1 ; *c1cn(*)nn1]

Embodiment 145. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 144, wherein:
   each m is independently selected from 1, 2, 3, 4, 5, 6, 7, 8, 9 and 10.
Embodiment 146. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 144, wherein:
   each m is independently selected from 1, 2, 3, 4, and 5.
Embodiment 147. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 144, wherein:
   each m is independently selected from 1, 2 and 3.
Embodiment 148. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 147, wherein:
   each n is independently selected from 1, 2, 3, 4, 5, 6, 7, 8, 9 and 10.
Embodiment 149. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 147, wherein:
   each n is independently selected from 1, 2, 3, 4 and 5.
Embodiment 150. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 147, wherein:
   each n is independently selected from 1, 2 and 3.
Embodiment 151. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 150, wherein:
   each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30.
Embodiment 152. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 150, wherein:
   each t is independently selected from 6, 7, 8, 9, 10, 11, 12, 13, 14, 15, 16, 17, 18, 19, 20, 21, 22, 23, 24, 25, 26, 27, 28, 29 and 30.
Embodiment 153. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 150, wherein:
   each t is independently selected from 4, 5, 6, 7, 8, 9, 10, 11, 12, 13, 14, 15, 16, 17, 18, 19, 20, 21, 22, 23, 24 or 25.
Embodiment 154. The compound of Formula (I) or any one of Embodiments 1 to 17, or pharmaceutically acceptable salt thereof, the linker of Formula (V) or any one of Embodiments 32 to 46, and the immunoconjugate of Formula (III) or any one of Embodiments 60 to 72, or any one of Embodiments 86 to 150, wherein:
   each t is independently selected from 1, 2, 3, 4, 5, 6, 7, 8, 9, 10, 11, 12, 13, 14, 15, 16, 17 and 18.
Embodiment 155. The immunoconjugate of Formula (III) or any one of Embodiments 60 to 72, or any one of Embodiments 86 to 154, wherein y is 1, 2, 3, 4, 5, 6, 7, 8, 9, 10, 11, 12, 13, 14, 15 or 16.
Embodiment 156. The immunoconjugate of Formula (III) or any one of Embodiments 60 to 72, or any one of Embodiments 86 to 154, wherein y is 1, 2, 3, 4, 5, 6, 7, 8, 9, 10, 11, 12, 13 or 14.
Embodiment 157. The immunoconjugate of Formula (III) or any one of Embodiments 60 to 72, or any one of Embodiments 86 to 154, wherein y is 1, 2, 3, 4, 5, 6, 7, 8, 9, 10, 11 or 12.
Embodiment 158. The immunoconjugate of Formula (III) or any one of Embodiments 60 to 72, or any one of Embodiments 86 to 154, wherein y is 1, 2, 3, 4, 5, 6, 7, 8, 9 or 10.
Embodiment 159. The immunoconjugate of Formula (III) or any one of Embodiments 60 to 72, or any one of Embodiments 86 to 154, wherein y is 1, 2, 3, 4, 5, 6, 7 or 8.
Embodiment 160. The immunoconjugate of Formula (III) or any one of Embodiments 60 to 72, or any one of Embodiments 86 to 154, wherein y is 1, 2, 3, 4, 5 or 6.
Embodiment 161. The immunoconjugate of Formula (III) or any one of Embodiments 60 to 72, or any one of Embodiments 86 to 154, wherein y is 1, 2, 3 or 4.
Embodiment 162. The immunoconjugate of Formula (III) or any one of Embodiments 60 to 72, or any one of Embodiments 86 to 154, wherein y is 1 or 2.
Embodiment 163. The immunoconjugate of Formula (III) or any one of Embodiments 60 to 72, or any one of Embodiments 86 to 154, wherein y is 2.
Embodiment 164. The immunoconjugate of Formula (III) or any one of Embodiments 60 to 72, or any one of Embodiments 86 to 154, wherein y is 4.
Embodiment 165. The immunoconjugate of Formula (III) or any one of Embodiments 60 to 72, or any one of Embodiments 86 to 154, wherein y is 6.
Embodiment 166. The immunoconjugate of Formula (III) or any one of Embodiments 60 to 72, or any one of Embodiments 86 to 154, wherein y is 8.
Embodiment 167. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a Drug moiety.
Embodiment 168. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a Drug moiety comprising an N or an O, wherein D is connected to A via a direct bond from A to the N or the O of the Drug moiety.
Embodiment 169. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety.
Embodiment 170. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety comprising an N or an O, wherein D is connected to A via a direct bond from A to the N or the O of the Drug moiety (e.g., D can be a quaternary ammonium when connected to A).
Embodiment 171. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 1.5 to 7.
Embodiment 172. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 1.5 to 6.
Embodiment 173. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 1.5 to 5.
Embodiment 174. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 1.5 to 4.
Embodiment 175. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 1.5 to 3.
Embodiment 176. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 1.5 to 2.
Embodiment 177. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 2 to 7.
Embodiment 178. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 2 to 6.
Embodiment 179. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 2 to 5.
Embodiment 180. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 2 to 4.
Embodiment 181. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is a hydrophobic Drug moiety having a S log P value of 2 to 3.
Embodiment 182. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is an auristatin.
Embodiment 183. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is

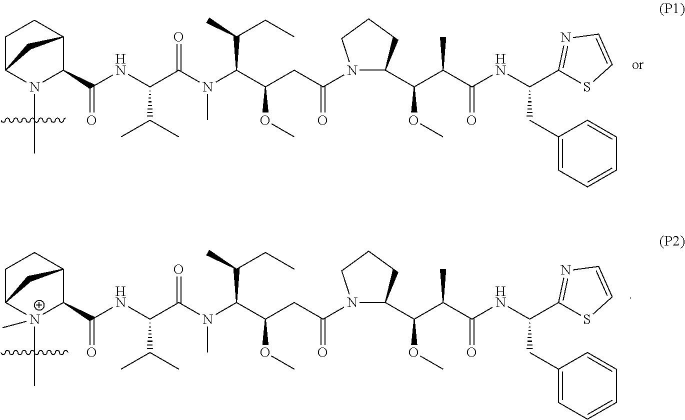 [SMILES: *N1[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C ; *[N+]1(C)[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C]

Embodiment 184. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is not a MCL-1 inhibitor.
Embodiment 185. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is not a BCL-2 inhibitor.
Embodiment 186. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein D is not a BCL-XL inhibitor.
Embodiment 187. The compound of Formula (I) or any one of Embodiments 1 to 17, or pharmaceutically acceptable salt thereof, or the immunoconjugate of Formula (III) or any one of Embodiments 60 to 72, or any one of Embodiments 86 to 166, wherein the linker in Formula (I) (i.e., the portion without D) or the linker in Formula (III) (i.e., the portion connecting Ab and D) is a linker selected from L2 through L208 disclosed herein, e.g., in tables 4A-4C, such as

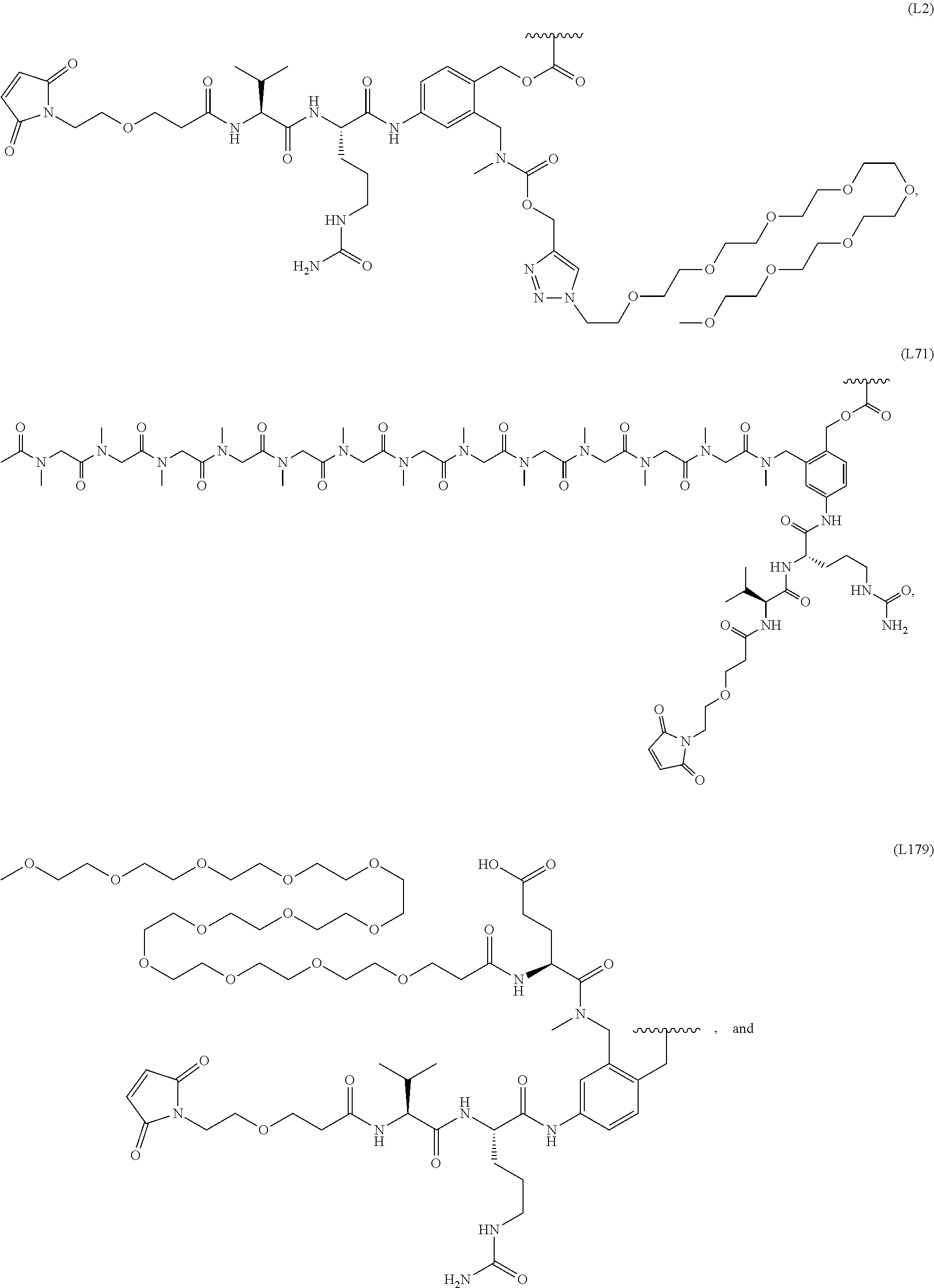 [SMILES: *C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOC)nn1 ; *C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; *Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCC(=O)O)NC(=O)CCOCCOCCOCCOCCOCCOCCOCCOCCOCCOCCOCCOC]

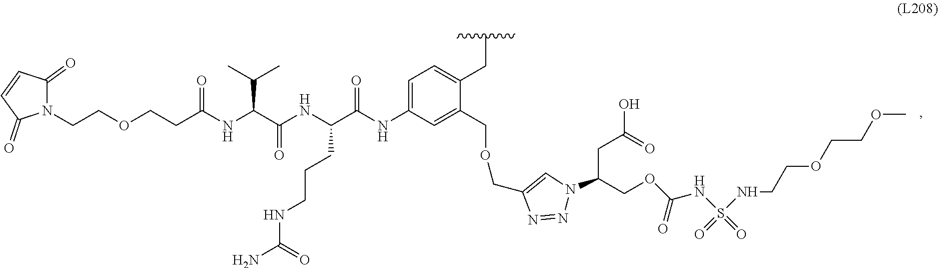 [SMILES: *Cc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)NS(=O)(=O)NCCOCCOC)CC(=O)O)nn1]

wherein the wavy line indicates the attachment point to D.
Embodiment 188. The linker of the Linker-Drug group of Formula (I) having the structure of Formula (V) or any one of embodiments 32 to 46, derived from any linker selected from L2 through L208 described herein, e.g., such as

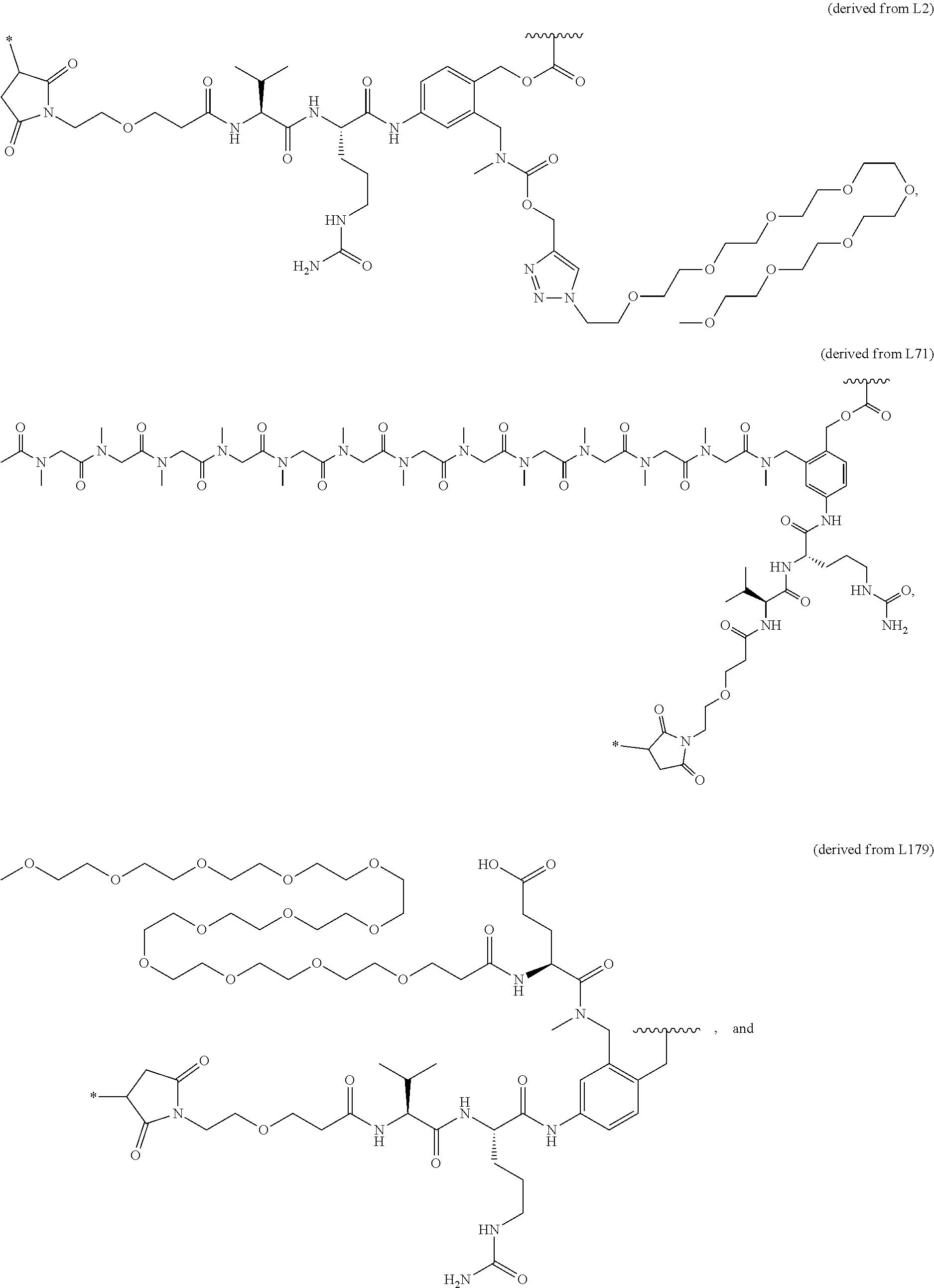

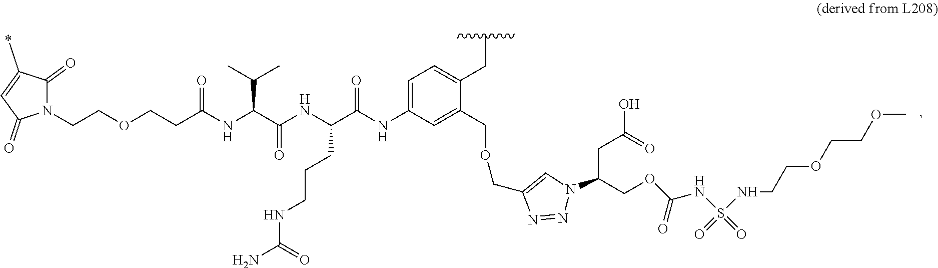 [SMILES: *Cc1ccc(NC(=O)[C@H](CCCNC(N)=O)NC(=O)[C@@H](NC(=O)CCOCCN2C(=O)C=C(*)C2=O)C(C)C)cc1COCc1cn([C@H](COC(=O)NS(=O)(=O)NCCOCCOC)CC(=O)O)nn1]

wherein the wavy line indicates the attachment point to D and * indicates the attachment point to an antibody or fragment thereof.

### Methods of Conjugation

The present invention provides various methods of conjugating Linker-Drug groups of the invention to antibodies or antibody fragments to produce Antibody Drug Conjugates which comprise a linker having one or more hydrophilic moieties.

A general reaction scheme for the formation of Antibody Drug Conjugates of Formula (III) is shown in Scheme 2 below:

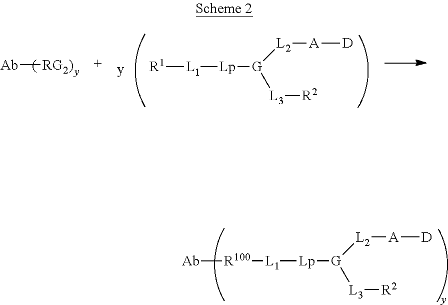

where: RG₂ is a reactive group which reacts with a compatible R¹ group to form a corresponding R¹⁰⁰ group (such groups are illustrated in Table 1). D, R¹, L₁, Lp, Ab, y and R¹⁰⁰ are as defined herein.

Scheme 3 further illustrates this general approach for the formation of Antibody Drug Conjugates of Formula (III), wherein the antibody comprises reactive groups (RG₂) which react with an R¹ group (as defined herein) to covalently attach the Linker-Drug group to the antibody via an R¹⁰⁰ group (as defined herein). For illustrative purposes only Scheme 3 shows the antibody having four RG₂ groups.

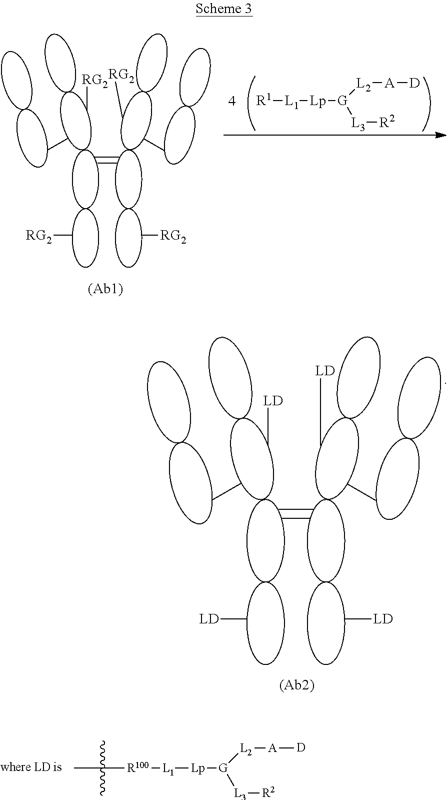

In one aspect, Linker-Drug groups are conjugated to antibodies via modified cysteine residues in the antibodies (see for example WO2014/124316). Scheme 4 illustrates this approach for the formation of Antibody Drug Conjugates of Formula (III) wherein a free thiol group generated from the engineered cysteine residues in the antibody react with an R¹ group (where R¹ is a maleimide) to covalently attach the Linker-Drug group to the antibody via an R¹⁰⁰ group (where R¹⁰⁰ is a succinimide ring). For illustrative purposes only Scheme 4 shows the antibody having four free thiol groups.

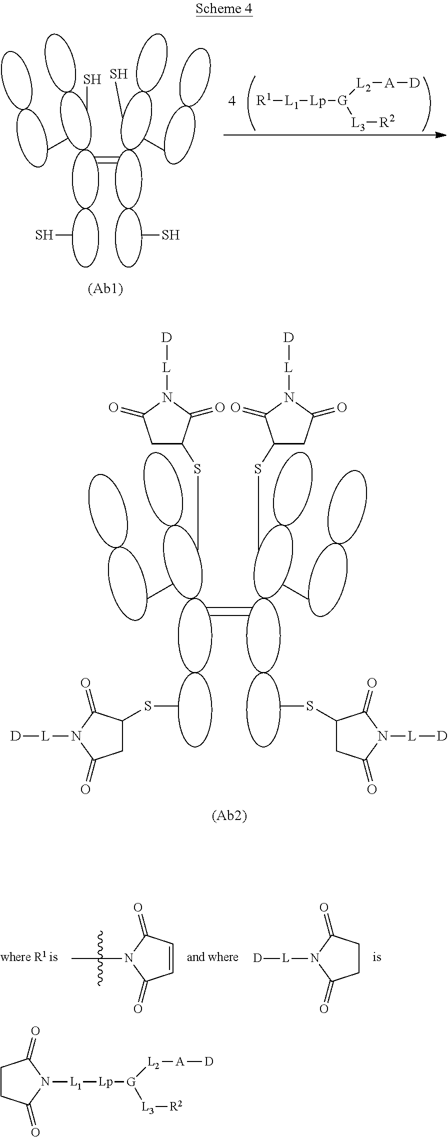

In another aspect, Linker-Drug groups are conjugated to antibodies via lysine residues in the antibodies. Scheme 5 illustrates this approach for the formation of Antibody Drug Conjugates of Formula (III) wherein a free amine group from the lysine residues in the antibody react with an R¹ group (where R¹ is an NHS ester, a pentafluorophenyl or a tetrafluorophenyl) to covalently attach the Linker-Drug group to the antibody via an R¹⁰⁰ group (where R¹⁰⁰ is an amide). For illustrative purposes only Scheme 5 shows the antibody having four amine groups.

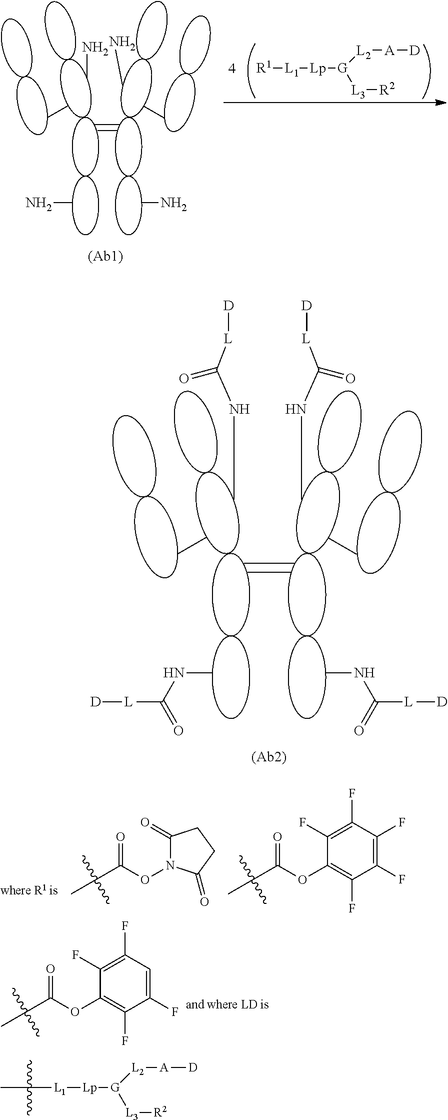

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 6 illustrates this approach for the formation of Antibody Drug Conjugates of Formula (III).

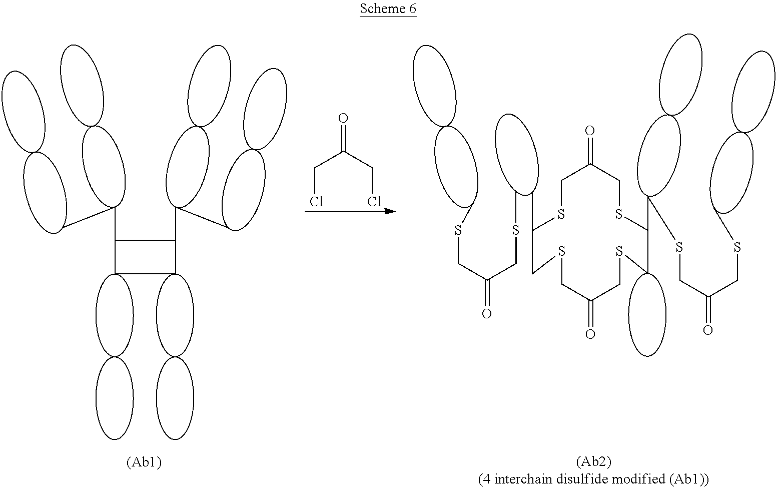

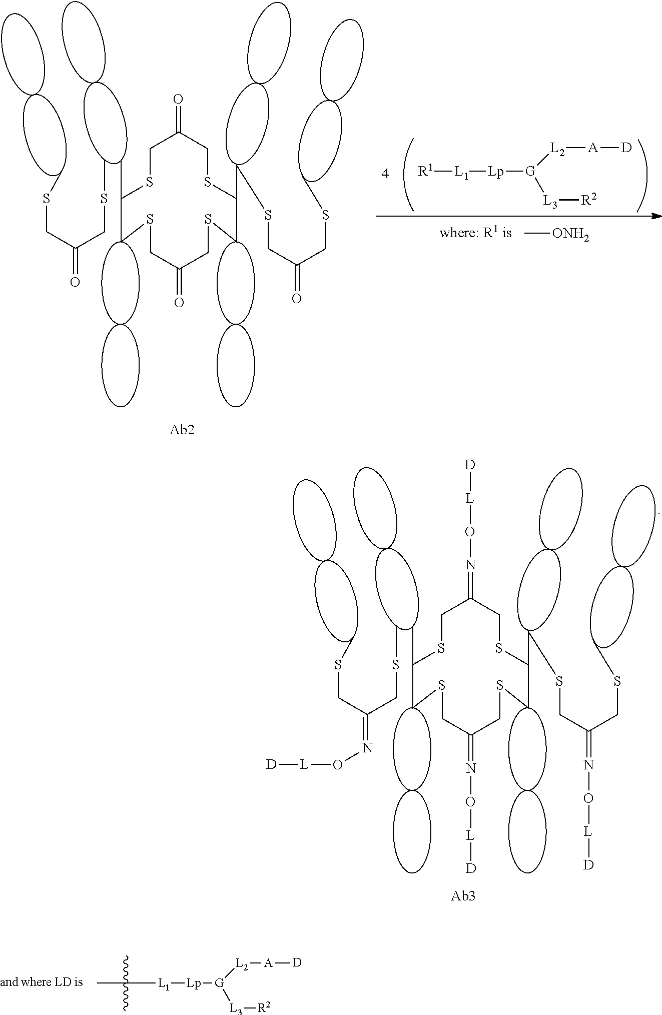

A general reaction scheme for the formation of Antibody Drug Conjugates of Formula (IV) is shown in Scheme 7 below:

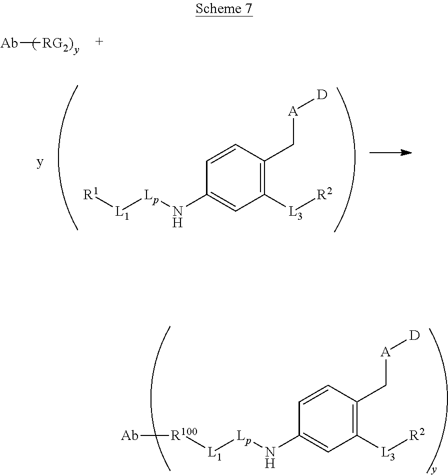

where: RG₂ is a reactive group which reacts with a compatible R¹ group to form a corresponding R¹⁰⁰ group (such groups are illustrated in Table 1). D, R¹, L₁, Lp, Ab, y and R¹⁰⁰ are as defined herein.

Scheme 8 further illustrates this general approach for the formation of Antibody Drug Conjugates of Formula (IV), wherein the antibody comprises reactive groups (RG₂) which react with an R¹ group (as defined herein) to covalently attach the Linker-Drug group to the antibody via an R¹⁰⁰ group (as defined herein). For illustrative purposes only Scheme 8 shows the antibody having four RG₂ groups.

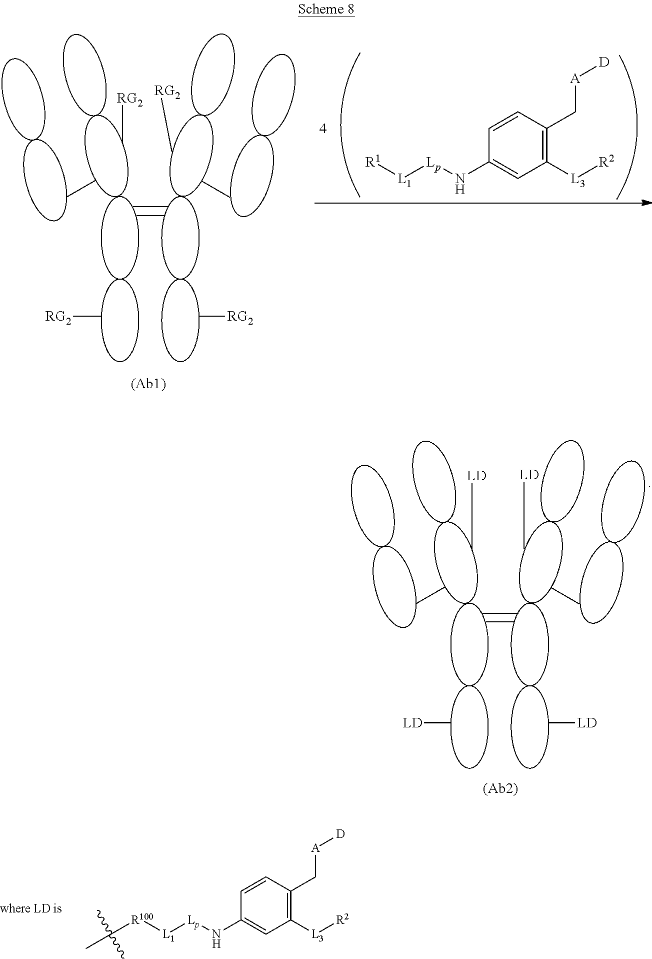

In one aspect, Linker-Drug groups are conjugated to antibodies via modified cysteine residues in the antibodies (see for example WO2014/124316). Scheme 9 illustrates this approach for the formation of Antibody Drug Conjugates of Formula (IV) wherein a free thiol group generated from the engineered cysteine residues in the antibody react with an R¹ group (where R¹ is a maleimide) to covalently attach the Linker-Drug group to the antibody via an R¹⁰⁰ group (where R¹⁰⁰ is a succinimide ring). For illustrative purposes only Scheme 9 shows the antibody having four free thiol groups.

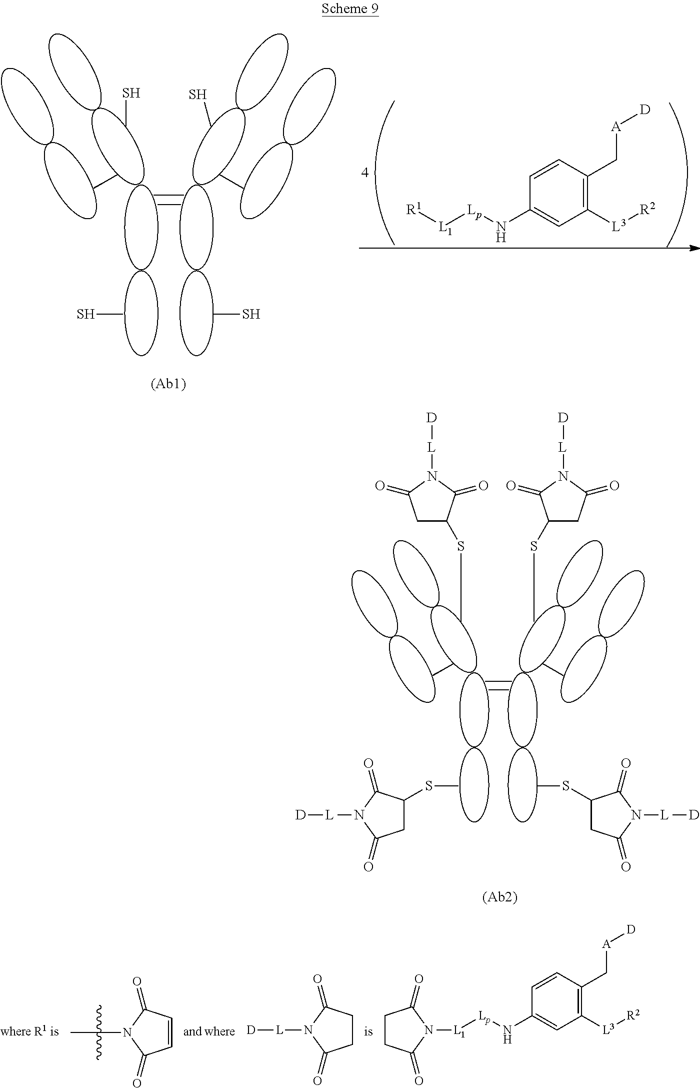

In another aspect, Linker-Drug groups are conjugated to antibodies via lysine residues in the antibodies. Scheme 10 illustrates this approach for the formation of Antibody Drug Conjugates of Formula (IV) wherein a free amine group from the lysine residues in the antibody react with an R¹ group (where R¹ is an NHS ester, a pentafluorophenyl or a tetrafluorophenyl) to covalently attach the Linker-Drug group to the antibody via an R¹⁰⁰ group (where R¹⁰⁰ is an amide). For illustrative purposes only Scheme 10 shows the antibody having four amine groups.

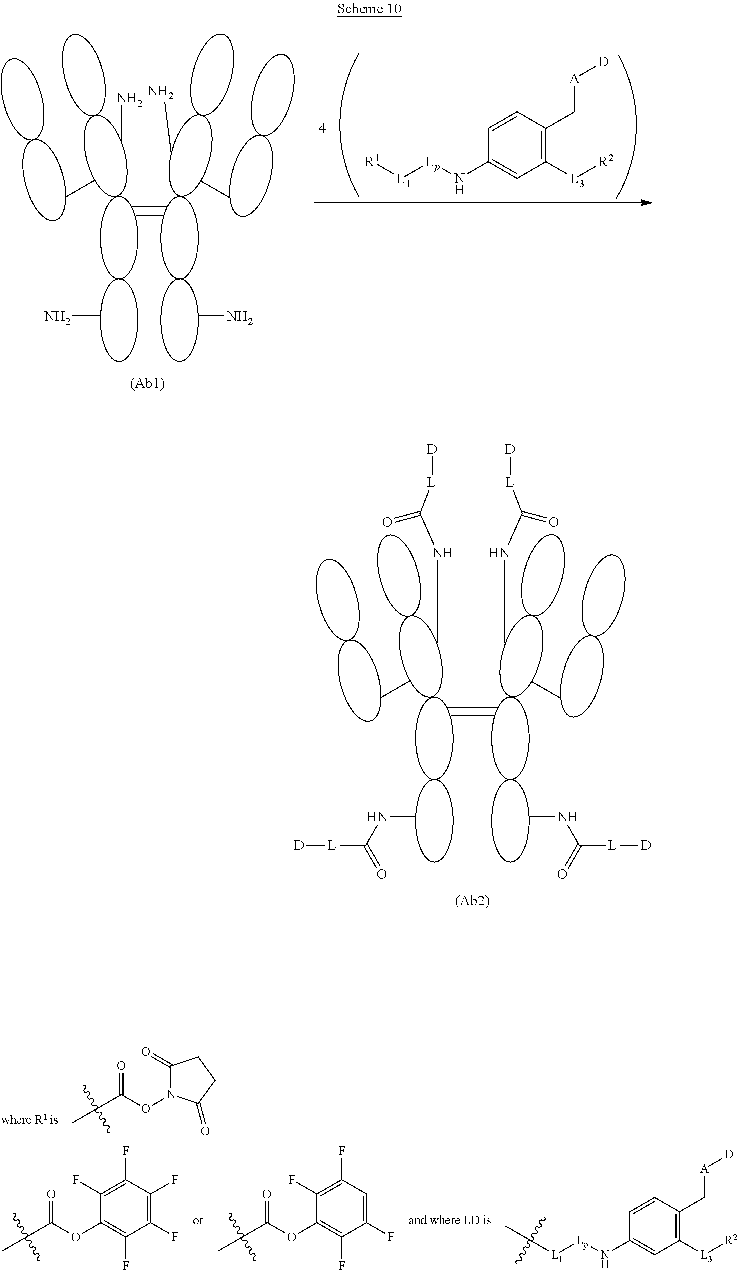

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 11 illustrates this approach for the formation of Antibody Drug Conjugates of Formula (IV).

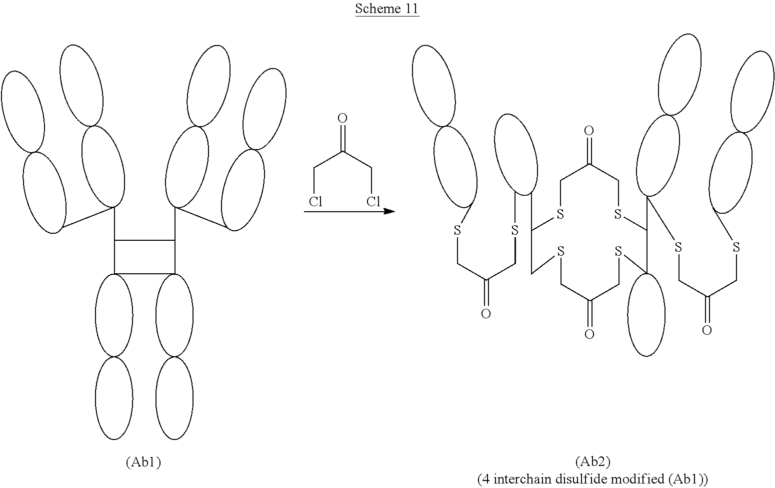

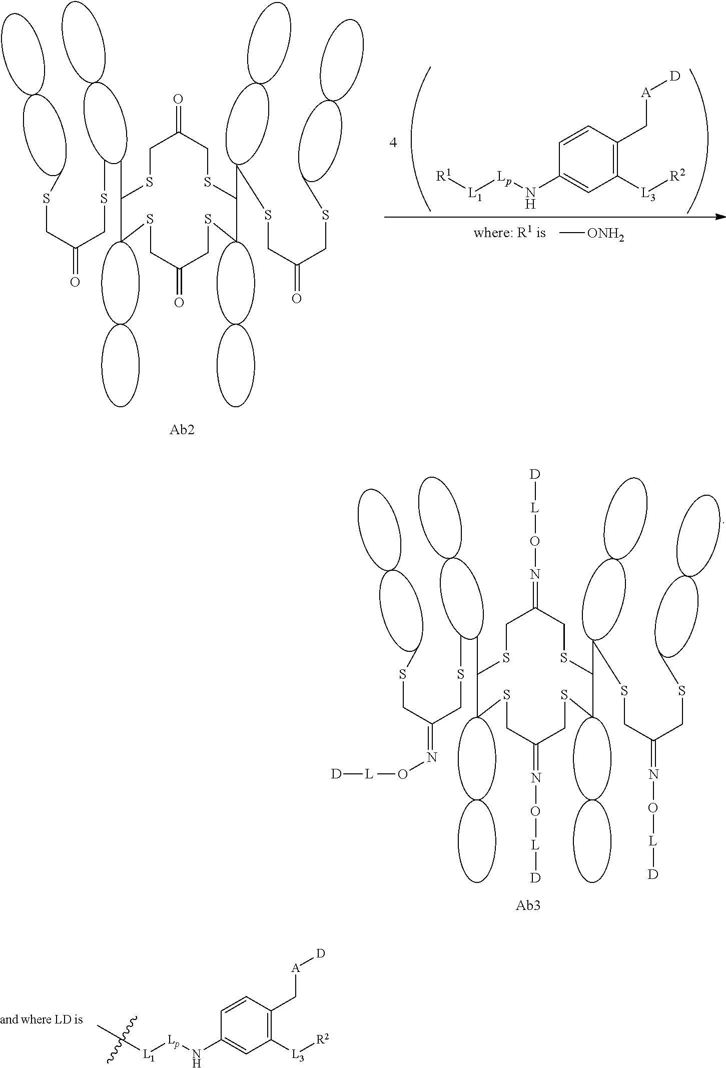

Provided are also protocols for some aspects of analytical methodology for evaluating antibody conjugates of the invention. Such analytical methodology and results can demonstrate that the conjugates have favorable properties, for example properties that would make them easier to manufacture, easier to administer to patients, more efficacious, and/or potentially safer for patients. One example is the determination of molecular size by size exclusion chromatography (SEC) wherein the amount of desired antibody species in a sample is determined relative to the amount of high molecular weight contaminants (e.g., dimer, multimer, or aggregated antibody) or low molecular weight contaminants (e.g., antibody fragments, degradation products, or individual antibody chains) present in the sample. In general, it is desirable to have higher amounts of monomer and lower amounts of, for example, aggregated antibody due to the impact of, for example, aggregates on other properties of the antibody sample such as but not limited to clearance rate, immunogenicity, and toxicity. A further example is the determination of the hydrophobicity by hydrophobic interaction chromatography (HIC) wherein the hydrophobicity of a sample is assessed relative to a set of standard antibodies of known properties. In general, it is desirable to have low hydrophobicity due to the impact of hydrophobicity on other properties of the antibody sample such as but not limited to aggregation, aggregation overtime, adherence to surfaces, hepatotoxicity, clearance rates, and pharmacokinetic exposure. See Damle, N. K., Nat Biotechnol. 2008; 26(8):884-885; Singh, S. K., Pharm Res. 2015; 32(11):3541-71. When measured by hydrophobic interaction chromatography, higher hydrophobicity index scores (i.e. elution from HIC column faster) reflect lower hydrophobicity of the conjugates. As shown in Examples below, a majority of the tested antibody conjugates showed a hydrophobicity index of greater than 0.8. In some embodiments, provided are antibody conjugates having a hydrophobicity index of 0.8 or greater, as determined by hydrophobic interaction chromatography.

### Antibodies

The present invention provides antibody conjugates that include antibodies or antibody fragments (e.g., antigen binding fragments) that specifically bind to an antigen, for example, a tumor antigen. Antibodies or antibody fragments (e.g., antigen binding fragments) of the invention include, but are not limited to, the human monoclonal antibodies or fragments thereof, isolated as described in the Examples.

The present invention in certain embodiments provides antibody conjugates that include antibodies or antibody fragments (e.g., antigen binding fragments) that specifically bind P-cadherin, said antibodies or antibody fragments (e.g., antigen binding fragments) comprise a VH domain having an amino acid sequence of SEQ ID NO: 7, 27, 47, 67, 87, 107, or 154. The present invention in certain embodiments also provides antibody conjugates that include antibodies or antibody fragments (e.g., antigen binding fragments) that specifically bind to P-cadherin, said antibodies or antibody fragments (e.g., antigen binding fragments) comprise a VH CDR having an amino acid sequence of any one of the VH CDRs listed in Table 3, infra. In particular embodiments, the invention provides antibody conjugates that include antibodies or antibody fragments (e.g., antigen binding fragments) that specifically bind to P-cadherin, said antibodies comprising (or alternatively, consist of) one, two, three, four, five or more VH CDRs having an amino acid sequence of any of the VH CDRs listed in Table 3, infra.

The present invention provides antibody conjugates that include antibodies or antibody fragments (e.g., antigen binding fragments) that specifically bind to P-cadherin, said antibodies or antibody fragments (e.g., antigen binding fragments) comprise a VL domain having an amino acid sequence of SEQ ID NO: 17, 37, 57, 77, 97, 117, or 166. The present invention also provides antibody conjugates that include antibodies or antibody fragments (e.g., antigen binding fragments) that specifically bind to P-cadherin, said antibodies or antibody fragments (e.g., antigen binding fragments) comprise a VL CDR having an amino acid sequence of any one of the VL CDRs listed in Table 3, infra. In particular, the invention provides antibody conjugates that include antibodies or antibody fragments (e.g., antigen binding fragments) that specifically bind to P-cadherin, said antibodies or antibody fragments (e.g., antigen binding fragments) comprise (or alternatively, consist of) one, two, three or more VL CDRs having an amino acid sequence of any of the VL CDRs listed in Table 3, infra.

Other antibodies or antibody fragments (e.g., antigen binding fragments) of the invention include amino acids that have been mutated, yet have at least 60, 70, 80, 90 or 95 percent identity in the CDR regions with the CDR regions depicted in the sequences described in Table 3. In some embodiments, the antibodies comprise mutant amino acid sequences wherein no more than 1, 2, 3, 4 or 5 amino acids have been mutated in the CDR regions when compared with the CDR regions depicted in the sequence described in Table 3.

The present invention also provides antibody conjugates that include antibodies or antigen binding fragments thereof that comprise modifications in the constant regions of the heavy chain, light chain, or both the heavy and light chain wherein particular amino acid residues have mutated to cysteines, also referred to herein at “CysMab” or “Cys” antibodies. As discussed above, drug moieties may be conjugated site specifically and with control over the number of drug moieties (“DAR Controlled”) to cysteine residues on antibodies. Cysteine modifications to antibodies for the purposes of site specifically controlling immunoconjugation are disclosed, for example, in WO2014/124316, which is incorporated herein in its entirety.

In some embodiments, the antibodies have been modified at positions 152 and 375 of the heavy chain, wherein the positions are defined according to the EU numbering system. Namely, the modifications are E152C and S375C. In other embodiments, the antibodies have been modified at position 360 of the heavy chain and position 107 of the kappa light chain, wherein the positions are defined according to the EU numbering system. Namely, the modifications are K360C and K107C. The positions of these mutations are illustrated, for example, in the context of human IgG1 heavy chain and kappy light chain constant regions in SEQ ID NOS:148-150 in Table 3. Throughout Table 3, cysteine modifications from wild type sequences are shown with underlining.

The present invention also provides nucleic acid sequences that encode the VH, VL, the full length heavy chain, and the full length light chain of the antibodies that specifically bind to P-cadherin. Such nucleic acid sequences can be optimized for expression in mammalian cells.

TABLE 3
Examples of anti-P-cadherin Antibodies of the Present Invention

| | | |
|---|---|---|
| SEQ ID NO. | Description | Sequence |

P-Cad Mab2

| | | |
|---|---|---|
| SEQ ID NO: 1 | HCDR1 | SQSAAWN |
| | (Kabat) | |
| SEQ ID NO: 2 | HCDR2 | RIYYRSKWYNDYALSVKS |
| | (Kabat) | |
| SEQ ID NO: 3 | HCDR3 | GEGYGREGFAI |
| | (Kabat) | |
| SEQ ID NO: 4 | HCDR1 | GDSVSSQSA |
| | (Chothia) | |
| SEQ ID NO: 5 | HCDR2 | YYRSKWY |
| | (Chothia) | |
| SEQ ID NO: 6 | HCDR3 | GEGYGREGFAI |
| | (Chothia) | |
| SEQ ID NO: 7 | VH | QVQLQQSGPGLVKPSQTLSLTCAISGDSVSSQSAAWN |
| | | WIRQSPSRGLEWLGRIYYRSKWYNDYALSVKSRITINPD |
| | | TSKNQFSLQLNSVTPEDTAVYYCARGEGYGREGFAIWG |
| | | QGTLVTVSS |
| SEQ ID NO: 8 | DNA VH | CAGGTGCAGCTGCAGCAGTCAGGCCCTGGCCTGGTC |
| | | AAGCCTAGTCAGACCCTGAGCCTGACCTGCGCTATTA |
| | | GCGGCGATAGTGTGTCTAGTCAGTCAGCCGCCTGGA |
| | | ACTGGATTAGACAGTCACCCTCTAGGGGCCTGGAGT |
| | | GGCTGGGTAGAATCTACTATAGGTCTAAGTGGTATAA |
| | | CGACTACGCCCTGAGCGTGAAGTCTAGGATCACTATT |
| | | AACCCCGACACCTCTAAGAATCAGTTTAGCCTGCAGC |
| | | TGAATAGCGTGACCCCCGAGGACACCGCCGTCTACT |
| | | ACTGCGCTAGAGGCGAGGGCTACGGTAGAGAGGGCT |
| | | TCGCTATCTGGGGTCAGGGCACCCTGGTCACCGTGT |
| | | CTAGC |
| SEQ ID NO: | DNA VH | CAGGTGCAATTGCAGCAGAGCGGTCCGGGCCTGGTG |
| 151 | | AAACCGAGCCAGACCCTGAGCCTGACCTGCGCGATT |
| | | TCCGGAGATAGCGTGAGCTCTCAGTCTGCTGCTTGG |
| | | AACTGGATTCGTCAGAGCCCGAGCCGTGGCCTCGAG |
| | | TGGCTGGGCCGTATCTACTACCGTAGCAAATGGTACA |
| | | ACGACTATGCCTTGAGCGTGAAAAGCCGCATTACCAT |
| | | TAACCCGGATACTTCGAAAAACCAGTTTAGCCTGCAA |
| | | CTGAACAGCGTGACCCCGGAAGATACGGCCGTGTAT |
| | | TATTGCGCGCGTGGTGAAGGTTACGGTCGTGAAGGT |
| | | TTCGCTATCTGGGGCCAAGGCACCCTGGTGACTGTTA |
| | | GCTCA |
| SEQ ID NO: 9 | Heavy Chain | QVQLQQSGPGLVKPSQTLSLTCAISGDSVSSQSAAWN |
| | | WIRQSPSRGLEWLGRIYYRSKWYNDYALSVKSRITINPD |
| | | TSKNQFSLQLNSVTPEDTAVYYCARGEGYGREGFAIWG |
| | | QGTLVTVSSASTKGPSVFPLAPSSKSTSGGTAALGCLV |
| | | KDYFPEPVTVSWNSGALTSGVHTFPAVLQSSGLYSLSS |
| | | VVTVPSSSLGTQTYICNVNHKPSNTKVDKRVEPKSCDK |
| | | THTCPPCPAPELLGGPSVFLFPPKPKDTLMISRTPEVTC |
| | | VVVDVSHEDPEVKFNWYVDGVEVHNAKTKPREEQYNS |
| | | TYRVVSVLTVLHQDWLNGKEYKCKVSNKALPAPIEKTIS |
| | | KAKGQPREPQVYTLPPSREEMTKNQVSLTCLVKGFYPS |
| | | DIAVEWESNGQPENNYKTTPPVLDSDGSFFLYSKLTVD |
| | | KSRWQQGNVFSCSVMHEALHNHYTQKSLSLSPGK |
| SEQ ID NO: 10 | DNA Heavy | CAGGTGCAGCTGCAGCAGTCAGGCCCTGGCCTGGTC |
| | Chain | AAGCCTAGTCAGACCCTGAGCCTGACCTGCGCTATTA |
| | | GCGGCGATAGTGTGTCTAGTCAGTCAGCCGCCTGGA |
| | | ACTGGATTAGACAGTCACCCTCTAGGGGCCTGGAGT |
| | | GGCTGGGTAGAATCTACTATAGGTCTAAGTGGTATAA |
| | | CGACTACGCCCTGAGCGTGAAGTCTAGGATCACTATT |
| | | AACCCCGACACCTCTAAGAATCAGTTTAGCCTGCAGC |
| | | TGAATAGCGTGACCCCCGAGGACACCGCCGTCTACT |
| | | ACTGCGCTAGAGGCGAGGGCTACGGTAGAGAGGGCT |
| | | TCGCTATCTGGGGTCAGGGCACCCTGGTCACCGTGT |
| | | CTAGCGCTAGCACTAAGGGCCCAAGTGTGTTTCCCCT |
| | | GGCCCCCAGCAGCAAGTCTACTTCCGGCGGAACTGC |
| | | TGCCCTGGGTTGCCTGGTGAAGGACTACTTCCCCGA |
| | | GCCCGTGACAGTGTCCTGGAACTCTGGGGCTCTGAC |
| | | TTCCGGCGTGCACACCTTCCCCGCCGTGCTGCAGAG |
| | | CAGCGGCCTGTACAGCCTGAGCAGCGTGGTGACAGT |
| | | GCCCTCCAGCTCTCTGGGAACCCAGACCTATATCTGC |
| | | AACGTGAACCACAAGCCCAGCAACACCAAGGTGGAC |
| | | AAGAGAGTGGAGCCCAAGAGCTGCGACAAGACCCAC |
| | | ACCTGCCCCCCCTGCCCAGCTCCAGAACTGCTGGGA |
| | | GGGCCTTCCGTGTTCCTGTTCCCCCCCAAGCCCAAG |
| | | GACACCCTGATGATCAGCAGGACCCCCGAGGTGACC |
| | | TGCGTGGTGGTGGACGTGTCCCACGAGGACCCAGAG |
| | | GTGAAGTTCAACTGGTACGTGGACGGCGTGGAGGTG |
| | | CACAACGCCAAGACCAAGCCCAGAGAGGAGCAGTAC |
| | | AACAGCACCTACAGGGTGGTGTCCGTGCTGACCGTG |
| | | CTGCACCAGGACTGGCTGAACGGCAAAGAATACAAG |
| | | TGCAAAGTCTCCAACAAGGCCCTGCCAGCCCCAATC |
| | | GAAAAGACAATCAGCAAGGCCAAGGGCCAGCCACGG |
| | | GAGCCCCAGGTGTACACCCTGCCCCCCAGCCGGGA |
| | | GGAGATGACCAAGAACCAGGTGTCCCTGACCTGTCT |
| | | GGTGAAGGGCTTCTACCCCAGCGATATCGCCGTGGA |
| | | GTGGGAGAGCAACGGCCAGCCCGAGAACAACTACAA |
| | | GACCACCCCCCCAGTGCTGGACAGCGACGGCAGCTT |
| | | CTTCCTGTACAGCAAGCTGACCGTGGACAAGTCCAG |
| | | GTGGCAGCAGGGCAACGTGTTCAGCTGCAGCGTGAT |
| | | GCACGAGGCCCTGCACAACCACTACACCCAGAAGTC |
| | | CCTGAGCCTGAGCCCCGGCAAG |
| SEQ ID NO: | E152C/S375 | QVQLQQSGPGLVKPSQTLSLTCAISGDSVSSQSAAWN |
| 130 | C CysMab | WIRQSPSRGLEWLGRIYYRSKWYNDYALSVKSRITINPD |
| | Mutated | TSKNQFSLQLNSVTPEDTAVYYCARGEGYGREGFAIWG |
| | Heavy Chain | QGTLVTVSSASTKGPSVFPLAPSSKSTSGGTAALGCLV |
| | | KDYFPCPVTVSWNSGALTSGVHTFPAVLQSSGLYSLSS |
| | | VVTVPSSSLGTQTYICNVNHKPSNTKVDKRVEPKSCDK |
| | | THTCPPCPAPELLGGPSVFLFPPKPKDTLMISRTPEVTC |
| | | VVVDVSHEDPEVKFNWYVDGVEVHNAKTKPREEQYNS |
| | | TYRVVSVLTVLHQDWLNGKEYKCKVSNKALPAPIEKTIS |
| | | KAKGQPREPQVYTLPPSREEMTKNQVSLTCLVKGFYPC |
| | | DIAVEWESNGQPENNYKTTPPVLDSDGSFFLYSKLTVD |
| | | KSRWQQGNVFSCSVMHEALHNHYTQKSLSLSPGK |
| SEQ ID NO: | DNA | CAGGTGCAATTGCAGCAGAGCGGTCCGGGCCTGGTG |
| 152 | E152C/S375 | AAACCGAGCCAGACCCTGAGCCTGACCTGCGCGATT |
| | C CysMab | TCCGGAGATAGCGTGAGCTCTCAGTCTGCTGCTTGG |
| | Mutated | AACTGGATTCGTCAGAGCCCGAGCCGTGGCCTCGAG |
| | Heavy Chain | TGGCTGGGCCGTATCTACTACCGTAGCAAATGGTACA |
| | | ACGACTATGCCTTGAGCGTGAAAAGCCGCATTACCAT |
| | | TAACCCGGATACTTCGAAAAACCAGTTTAGCCTGCAA |
| | | CTGAACAGCGTGACCCCGGAAGATACGGCCGTGTAT |
| | | TATTGCGCGCGTGGTGAAGGTTACGGTCGTGAAGGT |
| | | TTCGCTATCTGGGGCCAAGGCACCCTGGTGACTGTTA |
| | | GCTCAGCCTCTACGAAAGGCCCAAGCGTATTTCCCCT |
| | | GGCTCCTTCTAGTAAATCAACCTCAGGTGGTACAGCA |
| | | GCCCTTGGCTGCCTGGTCAAAGACTATTTCCCCTGTC |
| | | CGGTGACCGTCTCATGGAACTCAGGTGCTTTGACATC |
| | | TGGTGTGCATACATTCCCAGCTGTGCTGCAAAGTAGT |
| | | GGACTGTACAGCCTTTCCAGCGTGGTCACGGTGCCA |
| | | AGTAGCTCCTTGGGTACTCAGACTTATATCTGCAATG |
| | | TGAACCACAAGCCCTCTAACACGAAGGTGGACAAGC |
| | | GCGTGGAGCCCAAATCTTGCGATAAGACGCATACTTG |
| | | TCCCCCATGCCCTGCTCCTGAGCTGTTGGGAGGCCC |
| | | GTCAGTGTTCTTGTTCCCTCCGAAGCCTAAGGACACT |
| | | TTGATGATAAGTAGGACACCAGAGGTGACTTGCGTGG |
| | | TGGTTGATGTGTCCCATGAAGATCCCGAGGTCAAATT |
| | | TAATTGGTACGTAGATGGTGTCGAAGTTCACAATGCT |
| | | AAGACTAAGCCAAGGGAAGAGCAGTACAACAGTACAT |
| | | ATAGGGTAGTCTCCGTGCTGACAGTCCTCCACCAGG |
| | | ACTGGTTGAACGGCAAGGAATACAAATGTAAGGTGTC |
| | | AAACAAAGCTCTGCCTGCTCCCATTGAGAAAACAATC |
| | | TCTAAAGCCAAAGGCCAGCCGAGAGAGCCCCAAGTC |
| | | TACACTTTGCCCCCGAGCAGGGAGGAAATGACCAAG |
| | | AATCAGGTGAGTCTGACGTGCCTCGTCAAAGGATTTT |
| | | ATCCATGCGATATTGCAGTTGAATGGGAGAGCAATGG |
| | | CCAGCCAGAGAACAACTATAAAACCACACCACCCGTG |
| | | CTCGACTCTGATGGCAGCTTCTTCCTCTATAGCAAGC |
| | | TGACAGTCGATAAATCTCGCTGGCAGCAAGGCAATGT |
| | | GTTCTCCTGCTCCGTCATGCACGAGGCTTTGCATAAC |
| | | CATTATACTCAAAAATCTCTGTCCCTGTCACCTGGTAA |
| | | A |
| SEQ ID NO: | K360C | QVQLQQSGPGLVKPSQTLSLTCAISGDSVSSQSAAWN |
| 131 | CysMab | WIRQSPSRGLEWLGRIYYRSKWYNDYALSVKSRITINPD |
| | Mutated | TSKNQFSLQLNSVTPEDTAVYYCARGEGYGREGFAIWG |
| | Heavy Chain | QGTLVTVSSASTKGPSVFPLAPSSKSTSGGTAALGCLV |
| | | KDYFPEPVTVSWNSGALTSGVHTFPAVLQSSGLYSLSS |
| | | VVTVPSSSLGTQTYICNVNHKPSNTKVDKRVEPKSCDK |
| | | THTCPPCPAPELLGGPSVFLFPPKPKDTLMISRTPEVTC |
| | | VVVDVSHEDPEVKFNWYVDGVEVHNAKTKPREEQYNS |
| | | TYRVVSVLTVLHQDWLNGKEYKCKVSNKALPAPIEKTIS |
| | | KAKGQPREPQVYTLPPSREEMTCNQVSLTCLVKGFYPS |
| | | DIAVEWESNGQPENNYKTTPPVLDSDGSFFLYSKLTVD |
| | | KSRWQQGNVFSCSVMHEALHNHYTQKSLSLSPGK |
| SEQ ID NO: 11 | LCDR1 | RASQTISNTLA |
| | (Kabat) | |
| SEQ ID NO: 12 | LCDR2 | AASNLQS |
| | (Kabat) | |
| SEQ ID NO: 13 | LCDR3 | QQYLSWFT |
| | (Kabat) | |
| SEQ ID NO: 14 | LCDR1 | SQTISNT |
| | (Chothia) | |
| SEQ ID NO: 15 | LCDR2 | AAS |
| | (Chothia) | |
| SEQ ID NO: 16 | LCDR3 | YLSWF |
| | (Chothia) | |
| SEQ ID NO: 17 | VL | DIQMTQSPSSLSASVGDRVTITCRASQTISNTLAWYQQK |
| | | PGKAPKLLIYAASNLQSGVPSRFSGSGSGTDFTLTISSL |
| | | QPEDFATYYCQQYLSWFTFGQGTKVEIK |
| SEQ ID NO: 18 | DNA VL | GATATTCAGATGACTCAGTCACCTAGTAGCCTGAGCG |
| | | CTAGTGTGGGCGATAGAGTGACTATCACCTGTAGAGC |
| | | CTCTCAGACTATCTCTAACACCCTGGCCTGGTATCAG |
| | | CAGAAGCCCGGTAAAGCCCCTAAGCTGCTGATCTAC |
| | | GCCGCCTCTAACCTGCAGTCAGGCGTGCCCTCTAGG |
| | | TTTAGCGGTAGCGGTAGTGGCACCGACTTCACCCTG |
| | | ACTATTAGTAGCCTGCAGCCCGAGGACTTCGCTACCT |
| | | ACTACTGTCAGCAGTACCTGAGCTGGTTCACCTTCGG |
| | | TCAGGGCACTAAGGTCGAGATTAAG |
| SEQ ID NO: | DNA VL | GATATCCAGATGACCCAGAGCCCGAGCAGCCTGAGC |
| 153 | | GCCAGCGTGGGCGATCGCGTGACCATTACCTGCAGA |
| | | GCCAGCCAGACTATTTCTAACACTCTGGCTTGGTACC |
| | | AGCAGAAACCGGGCAAAGCGCCGAAACTATTAATCTA |
| | | CGCTGCTTCTAACCTGCAAAGCGGCGTGCCGAGCCG |
| | | CTTTAGCGGCAGCGGATCCGGCACCGATTTCACCCT |
| | | GACCATTAGCTCTCTGCAACCGGAAGACTTTGCGACC |
| | | TATTATTGCCAGCAGTACCTGTCTTGGTTCACCTTTGG |
| | | CCAGGGCACGAAAGTTGAAATTAAA |
| SEQ ID NO: 19 | Light Chain | DIQMTQSPSSLSASVGDRVTITCRASQTISNTLAWYQQK |
| | | PGKAPKLLIYAASNLQSGVPSRFSGSGSGTDFTLTISSL |
| | | QPEDFATYYCQQYLSWFTFGQGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYAC |
| | | EVTHQGLSSPVTKSFNRGEC |
| SEQ ID NO 20: | DNA Light | GATATTCAGATGACTCAGTCACCTAGTAGCCTGAGCG |
| | Chain | CTAGTGTGGGCGATAGAGTGACTATCACCTGTAGAGC |
| | | CTCTCAGACTATCTCTAACACCCTGGCCTGGTATCAG |
| | | CAGAAGCCCGGTAAAGCCCCTAAGCTGCTGATCTAC |
| | | GCCGCCTCTAACCTGCAGTCAGGCGTGCCCTCTAGG |
| | | TTTAGCGGTAGCGGTAGTGGCACCGACTTCACCCTG |
| | | ACTATTAGTAGCCTGCAGCCCGAGGACTTCGCTACCT |
| | | ACTACTGTCAGCAGTACCTGAGCTGGTTCACCTTCGG |
| | | TCAGGGCACTAAGGTCGAGATTAAGCGTACGGTGGC |
| | | CGCTCCCAGCGTGTTCATCTTCCCCCCCAGCGACGA |
| | | GCAGCTGAAGAGCGGCACCGCCAGCGTGGTGTGCCT |
| | | GCTGAACAACTTCTACCCCCGGGAGGCCAAGGTGCA |
| | | GTGGAAGGTGGACAACGCCCTGCAGAGCGGCAACAG |
| | | CCAGGAGAGCGTCACCGAGCAGGACAGCAAGGACTC |
| | | CACCTACAGCCTGAGCAGCACCCTGACCCTGAGCAA |
| | | GGCCGACTACGAGAAGCATAAGGTGTACGCCTGCGA |
| | | GGTGACCCACCAGGGCCTGTCCAGCCCCGTGACCAA |
| | | GAGCTTCAACAGGGGCGAGTGC |
| SEQ ID NO: | DNA Light | GATATCCAGATGACCCAGAGCCCGAGCAGCCTGAGC |
| 154 | Chain | GCCAGCGTGGGCGATCGCGTGACCATTACCTGCAGA |
| | | GCCAGCCAGACTATTTCTAACACTCTGGCTTGGTACC |
| | | AGCAGAAACCGGGCAAAGCGCCGAAACTATTAATCTA |
| | | CGCTGCTTCTAACCTGCAAAGCGGCGTGCCGAGCCG |
| | | CTTTAGCGGCAGCGGATCCGGCACCGATTTCACCCT |
| | | GACCATTAGCTCTCTGCAACCGGAAGACTTTGCGACC |
| | | TATTATTGCCAGCAGTACCTGTCTTGGTTCACCTTTGG |
| | | CCAGGGCACGAAAGTTGAAATTAAACGTACGGTGGC |
| | | CGCTCCCAGCGTGTTCATCTTCCCCCCCAGCGACGA |
| | | GCAGCTGAAGAGCGGCACCGCCAGCGTGGTGTGCCT |
| | | GCTGAACAACTTCTACCCCCGGGAGGCCAAGGTGCA |
| | | GTGGAAGGTGGACAACGCCCTGCAGAGCGGCAACAG |
| | | CCAGGAAAGCGTCACCGAGCAGGACAGCAAGGACTC |
| | | CACCTACAGCCTGAGCAGCACCCTGACCCTGAGCAA |
| | | GGCCGACTACGAGAAGCACAAGGTGTACGCCTGCGA |
| | | GGTGACCCACCAGGGCCTGTCCAGCCCCGTGACCAA |
| | | GAGCTTCAACCGGGGCGAGTGT |
| SEQ ID NO: | K107C | DIQMTQSPSSLSASVGDRVTITCRASQTISNTLAWYQQK |
| 132 | CysMab | PGKAPKLLIYAASNLQSGVPSRFSGSGSGTDFTLTISSL |
| | Mutated Light | QPEDFATYYCQQYLSWFTFGQGTKVEICRTVAAPSVFIF |
| | Chain | PPSDEQLKSGTASVVCLLNNFYPREAKVQWKVDNALQS |
| | | GNSQESVTEQDSKDSTYSLSSTLTLSKADYEKHKVYAC |
| | | EVTHQGLSSPVTKSFNRGEC |

P-Cad Mab1

| | | |
|---|---|---|
| SEQ ID NO: 21 | HCDR1 | DHTIH |
| | (Kabat) | |
| SEQ ID NO: 22 | HCDR2 | YIYPRSGSINYNEKFKG |
| | (Kabat) | |
| SEQ ID NO: 23 | HCDR3 | RNLFLPMEY |
| | (Kabat) | |
| SEQ ID NO: 24 | HCDR1 | GYTFTDH |
| | (Chothia) | |
| SEQ ID NO: 25 | HCDR2 | YPRSGS |
| | (Chothia) | |
| SEQ ID NO: 26 | HCDR3 | RNLFLPMEY |
| | (Chothia) | |
| SEQ ID NO: 27 | VH | EVQLVQSGAEVKKPGESLKISCKVSGYTFTDHTIHWM |
| | | RQMPGKGLEWMGYIYPRSGSINYNEKFKGQVTISADK |
| | | SSSTAYLQWSSLKASDTAMYYCARRNLFLPMEYWGQ |
| | | GTLVTVSS |
| SEQ ID NO: 28 | DNA VH | GAGGTGCAGCTGGTGCAGTCAGGCGCCGAAGTGAA |
| | | GAAGCCCGGCGAGTCACTGAAGATTAGCTGTAAAG |
| | | TCTCAGGCTACACCTTCACCGATCACACTATTCACT |
| | | GGATGAGACAGATGCCCGGTAAAGGCCTGGAGTGG |
| | | ATGGGCTATATCTACCCTAGATCAGGCTCTATTAAC |
| | | TATAACGAGAAGTTTAAGGGTCAGGTCACAATTAGC |
| | | GCCGATAAGTCTAGCTCTACCGCCTACCTGCAGTG |
| | | GTCTAGCCTGAAGGCTAGTGACACCGCTATGTACTA |
| | | CTGCGCTAGACGTAACCTGTTCCTGCCTATGGAATA |
| | | CTGGGGTCAGGGCACCCTGGTCACCGTGTCTAGC |
| SEQ ID NO: 29 | Heavy Chain | EVQLVQSGAEVKKPGESLKISCKVSGYTFTDHTIHWM |
| | | RQMPGKGLEWMGYIYPRSGSINYNEKFKGQVTISADK |
| | | SSSTAYLQWSSLKASDTAMYYCARRNLFLPMEYWGQ |
| | | GTLVTVSSASTKGPSVFPLAPSSKSTSGGTAALGCLV |
| | | KDYFPEPVTVSWNSGALTSGVHTFPAVLQSSGLYSLS |
| | | SVVTVPSSSLGTQTYICNVNHKPSNTKVDKRVEPKSC |
| | | DKTHTCPPCPAPELLGGPSVFLFPPKPKDTLMISRTPE |
| | | VTCVVVDVSHEDPEVKFNWYVDGVEVHNAKTKPREE |
| | | QYNSTYRVVSVLTVLHQDWLNGKEYKCKVSNKALPA |
| | | PIEKTISKAKGQPREPQVYTLPPSREEMTKNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 30 | DNA Heavy | GAGGTGCAGCTGGTGCAGTCAGGCGCCGAAGTGAA |
| | Chain | GAAGCCCGGCGAGTCACTGAAGATTAGCTGTAAAG |
| | | TCTCAGGCTACACCTTCACCGATCACACTATTCACT |
| | | GGATGAGACAGATGCCCGGTAAAGGCCTGGAGTGG |
| | | ATGGGCTATATCTACCCTAGATCAGGCTCTATTAAC |
| | | TATAACGAGAAGTTTAAGGGTCAGGTCACAATTAGC |
| | | GCCGATAAGTCTAGCTCTACCGCCTACCTGCAGTG |
| | | GTCTAGCCTGAAGGCTAGTGACACCGCTATGTACTA |
| | | CTGCGCTAGACGTAACCTGTTCCTGCCTATGGAATA |
| | | CTGGGGTCAGGGCACCCTGGTCACCGTGTCTAGCG |
| | | CTAGCACTAAGGGCCCAAGTGTGTTTCCCCTGGCC |
| | | CCCAGCAGCAAGTCTACTTCCGGCGGAACTGCTGC |
| | | CCTGGGTTGCCTGGTGAAGGACTACTTCCCCGAGC |
| | | CCGTGACAGTGTCCTGGAACTCTGGGGCTCTGACT |
| | | TCCGGCGTGCACACCTTCCCCGCCGTGCTGCAGAG |
| | | CAGCGGCCTGTACAGCCTGAGCAGCGTGGTGACAG |
| | | TGCCCTCCAGCTCTCTGGGAACCCAGACCTATATCT |
| | | GCAACGTGAACCACAAGCCCAGCAACACCAAGGTG |
| | | GACAAGAGAGTGGAGCCCAAGAGCTGCGACAAGAC |
| | | CCACACCTGCCCCCCCTGCCCAGCTCCAGAACTGC |
| | | TGGGAGGGCCTTCCGTGTTCCTGTTCCCCCCCAAG |
| | | CCCAAGGACACCCTGATGATCAGCAGGACCCCCGA |
| | | GGTGACCTGCGTGGTGGTGGACGTGTCCCACGAG |
| | | GACCCAGAGGTGAAGTTCAACTGGTACGTGGACGG |
| | | CGTGGAGGTGCACAACGCCAAGACCAAGCCCAGAG |
| | | AGGAGCAGTACAACAGCACCTACAGGGTGGTGTCC |
| | | GTGCTGACCGTGCTGCACCAGGACTGGCTGAACGG |
| | | CAAAGAATACAAGTGCAAAGTCTCCAACAAGGCCCT |
| | | GCCAGCCCCAATCGAAAAGACAATCAGCAAGGCCA |
| | | AGGGCCAGCCACGGGAGCCCCAGGTGTACACCCT |
| | | GCCCCCCAGCCGGGAGGAGATGACCAAGAACCAG |
| | | GTGTCCCTGACCTGTCTGGTGAAGGGCTTCTACCC |
| | | CAGCGATATCGCCGTGGAGTGGGAGAGCAACGGC |
| | | CAGCCCGAGAACAACTACAAGACCACCCCCCCAGT |
| | | GCTGGACAGCGACGGCAGCTTCTTCCTGTACAGCA |
| | | AGCTGACCGTGGACAAGTCCAGGTGGCAGCAGGG |
| | | CAACGTGTTCAGCTGCAGCGTGATGCACGAGGCCC |
| | | TGCACAACCACTACACCCAGAAGTCCCTGAGCCTG |
| | | AGCCCCGGCAAG |
| SEQ ID NO: 133 | E152C/S375 | EVQLVQSGAEVKKPGESLKISCKVSGYTFTDHTIHWM |
| | C CysMab | RQMPGKGLEWMGYIYPRSGSINYNEKFKGQVTISADK |
| | Mutated | SSSTAYLQWSSLKASDTAMYYCARRNLFLPMEYWGQ |
| | Heavy Chain | GTLVTVSSASTKGPSVFPLAPSSKSTSGGTAALGCLV |
| | | KDYFPCPVTVSWNSGALTSGVHTFPAVLQSSGLYSLS |
| | | SVVTVPSSSLGTQTYICNVNHKPSNTKVDKRVEPKSC |
| | | DKTHTCPPCPAPELLGGPSVFLFPPKPKDTLMISRTPE |
| | | VTCVVVDVSHEDPEVKFNWYVDGVEVHNAKTKPREE |
| | | QYNSTYRVVSVLTVLHQDWLNGKEYKCKVSNKALPA |
| | | PIEKTISKAKGQPREPQVYTLPPSREEMTKNQVSLTCL |
| | | VKGFYPCDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 134 | K360C | EVQLVQSGAEVKKPGESLKISCKVSGYTFTDHTIHWM |
| | CysMab | RQMPGKGLEWMGYIYPRSGSINYNEKFKGQVTISADK |
| | Mutated | SSSTAYLQWSSLKASDTAMYYCARRNLFLPMEYWGQ |
| | Heavy Chain | GTLVTVSSASTKGPSVFPLAPSSKSTSGGTAALGCLV |
| | | KDYFPEPVTVSWNSGALTSGVHTFPAVLQSSGLYSLS |
| | | SVVTVPSSSLGTQTYICNVNHKPSNTKVDKRVEPKSC |
| | | DKTHTCPPCPAPELLGGPSVFLFPPKPKDTLMISRTPE |
| | | VTCVVVDVSHEDPEVKFNWYVDGVEVHNAKTKPREE |
| | | QYNSTYRVVSVLTVLHQDWLNGKEYKCKVSNKALPA |
| | | PIEKTISKAKGQPREPQVYTLPPSREEMTCNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 31 | LCDR1 | RSSQSLLSSGDQKNYLT |
| | (Kabat) | |
| SEQ ID NO: 32 | LCDR2 | WASTRES |
| | (Kabat) | |
| SEQ ID NO: 33 | LCDR3 | QNDYRYPLT |
| | (Kabat) | |
| SEQ ID NO: 34 | LCDR1 | SQSLLSSGDQKNY |
| | (Chothia) | |
| SEQ ID NO: 35 | LCDR2 | WAS |
| | (Chothia) | |
| SEQ ID NO: 36 | LCDR3 | DYRYPL |
| | (Chothia) | |
| SEQ ID NO: 37 | VL | DIVMTQTPLSLPVTPGEPASISCRSSQSLLSSGDQKNY |
| | | LTWYLQKPGQSPQLLIYWASTRESGVPDRFSGSGSG |
| | | TDFTLKISRVEAEDVGVYYCQNDYRYPLTFGQGTKLEI |
| | | K |
| SEQ ID NO: 38 | DNA VL | GATATCGTGATGACTCAGACCCCCCTGAGCCTGCC |
| | | CGTGACCCCTGGCGAGCCTGCCTCTATTAGCTGTA |
| | | GATCTAGTCAGTCACTGCTGTCTAGCGGCGATCAGA |
| | | AGAACTACCTGACCTGGTATCTGCAGAAGCCCGGT |
| | | CAGTCACCTCAGCTGCTGATCTACTGGGCCTCTACT |
| | | AGAGAATCAGGCGTGCCCGATAGGTTTAGCGGTAG |
| | | CGGTAGTGGCACCGACTTCACCCTGAAGATCTCTA |
| | | GGGTGGAAGCCGAGGACGTGGGCGTCTACTACTGT |
| | | CAGAACGACTATAGATACCCCCTGACCTTCGGTCAG |
| | | GGCACTAAGCTGGAGATTAAG |
| SEQ ID NO: 39 | Light Chain | DIVMTQTPLSLPVTPGEPASISCRSSQSLLSSGDQKNY |
| | | LTWYLQKPGQSPQLLIYWASTRESGVPDRFSGSGSG |
| | | TDFTLKISRVEAEDVGVYYCQNDYRYPLTFGQGTKLEI |
| | | KRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |
| SEQ ID NO 40: | DNA Light | GATATCGTGATGACTCAGACCCCCCTGAGCCTGCC |
| | Chain | CGTGACCCCTGGCGAGCCTGCCTCTATTAGCTGTA |
| | | GATCTAGTCAGTCACTGCTGTCTAGCGGCGATCAGA |
| | | AGAACTACCTGACCTGGTATCTGCAGAAGCCCGGT |
| | | CAGTCACCTCAGCTGCTGATCTACTGGGCCTCTACT |
| | | AGAGAATCAGGCGTGCCCGATAGGTTTAGCGGTAG |
| | | CGGTAGTGGCACCGACTTCACCCTGAAGATCTCTA |
| | | GGGTGGAAGCCGAGGACGTGGGCGTCTACTACTGT |
| | | CAGAACGACTATAGATACCCCCTGACCTTCGGTCAG |
| | | GGCACTAAGCTGGAGATTAAGCGTACGGTGGCCGC |
| | | TCCCAGCGTGTTCATCTTCCCCCCCAGCGACGAGC |
| | | AGCTGAAGAGCGGCACCGCCAGCGTGGTGTGCCT |
| | | GCTGAACAACTTCTACCCCCGGGAGGCCAAGGTGC |
| | | AGTGGAAGGTGGACAACGCCCTGCAGAGCGGCAAC |
| | | AGCCAGGAGAGCGTCACCGAGCAGGACAGCAAGG |
| | | ACTCCACCTACAGCCTGAGCAGCACCCTGACCCTG |
| | | AGCAAGGCCGACTACGAGAAGCATAAGGTGTACGC |
| | | CTGCGAGGTGACCCACCAGGGCCTGTCCAGCCCC |
| | | GTGACCAAGAGCTTCAACAGGGGCGAGTGC |
| SEQ ID NO: 135 | K107C | DIVMTQTPLSLPVTPGEPASISCRSSQSLLSSGDQKNY |
| | CysMab | LTWYLQKPGQSPQLLIYWASTRESGVPDRFSGSGSG |
| | Mutated | TDFTLKISRVEAEDVGVYYCQNDYRYPLTFGQGTKLEI |
| | Light Chain | CRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |

P-Cad Mab3

| | | |
|---|---|---|
| SEQ ID NO: 41 | HCDR1 | DHTIH |
| | (Kabat) | |
| SEQ ID NO: 42 | HCDR2 | YIYPRSGSINYNEKFKG |
| | (Kabat) | |
| SEQ ID NO: 43 | HCDR3 | RNLFLPMEY |
| | (Kabat) | |
| SEQ ID NO: 44 | HCDR1 | GYTFTDH |
| | (Chothia) | |
| SEQ ID NO: 45 | HCDR2 | YPRSGS |
| | (Chothia) | |
| SEQ ID NO: 46 | HCDR3 | RNLFLPMEY |
| | (Chothia) | |
| SEQ ID NO: 47 | VH | EVQLVQSGAEVKKPGESLKISCKVSGYTFTDHTIHWM |
| | | RQMPGKGLEWMGYIYPRSGSINYNEKFKGQVTISADK |
| | | SSSTAYLQWSSLKASDTAMYYCARRNLFLPMEYWGQ |
| | | GTLVTVSS |
| SEQ ID NO: 48 | DNA VH | GAGGTGCAGCTGGTGCAGTCAGGCGCCGAAGTGAA |
| | | GAAGCCCGGCGAGTCACTGAAGATTAGCTGTAAAG |
| | | TCTCAGGCTACACCTTCACCGATCACACTATTCACT |
| | | GGATGAGACAGATGCCCGGTAAAGGCCTGGAGTGG |
| | | ATGGGCTATATCTACCCTAGATCAGGCTCTATTAAC |
| | | TATAACGAGAAGTTTAAGGGTCAGGTCACAATTAGC |
| | | GCCGATAAGTCTAGCTCTACCGCCTACCTGCAGTG |
| | | GTCTAGCCTGAAGGCTAGTGACACCGCTATGTACTA |
| | | CTGCGCTAGACGTAACCTGTTCCTGCCTATGGAATA |
| | | CTGGGGTCAGGGCACCCTGGTCACCGTGTCTAGC |
| SEQ ID NO: 49 | Heavy Chain | EVQLVQSGAEVKKPGESLKISCKVSGYTFTDHTIHWM |
| | | RQMPGKGLEWMGYIYPRSGSINYNEKFKGQVTISADK |
| | | SSSTAYLQWSSLKASDTAMYYCARRNLFLPMEYWGQ |
| | | GTLVTVSSASTKGPSVFPLAPSSKSTSGGTAALGCLV |
| | | KDYFPEPVTVSWNSGALTSGVHTFPAVLQSSGLYSLS |
| | | SVVTVPSSSLGTQTYICNVNHKPSNTKVDKRVEPKSC |
| | | DKTHTCPPCPAPELLGGPSVFLFPPKPKDTLMISRTPE |
| | | VTCVVVDVSHEDPEVKFNWYVDGVEVHNAKTKPREE |
| | | QYNSTYRVVSVLTVLHQDWLNGKEYKCKVSNKALPA |
| | | PIEKTISKAKGQPREPQVYTLPPSREEMTKNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 50 | DNA Heavy | GAGGTGCAGCTGGTGCAGTCAGGCGCCGAAGTGAA |
| | Chain | GAAGCCCGGCGAGTCACTGAAGATTAGCTGTAAAG |
| | | TCTCAGGCTACACCTTCACCGATCACACTATTCACT |
| | | GGATGAGACAGATGCCCGGTAAAGGCCTGGAGTGG |
| | | ATGGGCTATATCTACCCTAGATCAGGCTCTATTAAC |
| | | TATAACGAGAAGTTTAAGGGTCAGGTCACAATTAGC |
| | | GCCGATAAGTCTAGCTCTACCGCCTACCTGCAGTG |
| | | GTCTAGCCTGAAGGCTAGTGACACCGCTATGTACTA |
| | | CTGCGCTAGACGTAACCTGTTCCTGCCTATGGAATA |
| | | CTGGGGTCAGGGCACCCTGGTCACCGTGTCTAGCG |
| | | CTAGCACTAAGGGCCCAAGTGTGTTTCCCCTGGCC |
| | | CCCAGCAGCAAGTCTACTTCCGGCGGAACTGCTGC |
| | | CCTGGGTTGCCTGGTGAAGGACTACTTCCCCGAGC |
| | | CCGTGACAGTGTCCTGGAACTCTGGGGCTCTGACT |
| | | TCCGGCGTGCACACCTTCCCCGCCGTGCTGCAGAG |
| | | CAGCGGCCTGTACAGCCTGAGCAGCGTGGTGACAG |
| | | TGCCCTCCAGCTCTCTGGGAACCCAGACCTATATCT |
| | | GCAACGTGAACCACAAGCCCAGCAACACCAAGGTG |
| | | GACAAGAGAGTGGAGCCCAAGAGCTGCGACAAGAC |
| | | CCACACCTGCCCCCCCTGCCCAGCTCCAGAACTGC |
| | | TGGGAGGGCCTTCCGTGTTCCTGTTCCCCCCCAAG |
| | | CCCAAGGACACCCTGATGATCAGCAGGACCCCCGA |
| | | GGTGACCTGCGTGGTGGTGGACGTGTCCCACGAG |
| | | GACCCAGAGGTGAAGTTCAACTGGTACGTGGACGG |
| | | CGTGGAGGTGCACAACGCCAAGACCAAGCCCAGAG |
| | | AGGAGCAGTACAACAGCACCTACAGGGTGGTGTCC |
| | | GTGCTGACCGTGCTGCACCAGGACTGGCTGAACGG |
| | | CAAAGAATACAAGTGCAAAGTCTCCAACAAGGCCCT |
| | | GCCAGCCCCAATCGAAAAGACAATCAGCAAGGCCA |
| | | AGGGCCAGCCACGGGAGCCCCAGGTGTACACCCT |
| | | GCCCCCCAGCCGGGAGGAGATGACCAAGAACCAG |
| | | GTGTCCCTGACCTGTCTGGTGAAGGGCTTCTACCC |
| | | CAGCGATATCGCCGTGGAGTGGGAGAGCAACGGC |
| | | CAGCCCGAGAACAACTACAAGACCACCCCCCCAGT |
| | | GCTGGACAGCGACGGCAGCTTCTTCCTGTACAGCA |
| | | AGCTGACCGTGGACAAGTCCAGGTGGCAGCAGGG |
| | | CAACGTGTTCAGCTGCAGCGTGATGCACGAGGCCC |
| | | TGCACAACCACTACACCCAGAAGTCCCTGAGCCTG |
| | | AGCCCCGGCAAG |
| SEQ ID NO: 136 | E152C/S375 | EVQLVQSGAEVKKPGESLKISCKVSGYTFTDHTIHWM |
| | C CysMab | RQMPGKGLEWMGYIYPRSGSINYNEKFKGQVTISADK |
| | Mutated | SSSTAYLQWSSLKASDTAMYYCARRNLFLPMEYWGQ |
| | Heavy Chain | GTLVTVSSASTKGPSVFPLAPSSKSTSGGTAALGCLV |
| | | KDYFPCPVTVSWNSGALTSGVHTFPAVLQSSGLYSLS |
| | | SVVTVPSSSLGTQTYICNVNHKPSNTKVDKRVEPKSC |
| | | DKTHTCPPCPAPELLGGPSVFLFPPKPKDTLMISRTPE |
| | | VTCVVVDVSHEDPEVKFNWYVDGVEVHNAKTKPREE |
| | | QYNSTYRVVSVLTVLHQDWLNGKEYKCKVSNKALPA |
| | | PIEKTISKAKGQPREPQVYTLPPSREEMTKNQVSLTCL |
| | | VKGFYPCDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 137 | K360C | EVQLVQSGAEVKKPGESLKISCKVSGYTFTDHTIHWM |
| | CysMab | RQMPGKGLEWMGYIYPRSGSINYNEKFKGQVTISADK |
| | Mutated | SSSTAYLQWSSLKASDTAMYYCARRNLFLPMEYWGQ |
| | Heavy Chain | GTLVTVSSASTKGPSVFPLAPSSKSTSGGTAALGCLV |
| | | KDYFPEPVTVSWNSGALTSGVHTFPAVLQSSGLYSLS |
| | | SVVTVPSSSLGTQTYICNVNHKPSNTKVDKRVEPKSC |
| | | DKTHTCPPCPAPELLGGPSVFLFPPKPKDTLMISRTPE |
| | | VTCVVVDVSHEDPEVKFNWYVDGVEVHNAKTKPREE |
| | | QYNSTYRVVSVLTVLHQDWLNGKEYKCKVSNKALPA |
| | | PIEKTISKAKGQPREPQVYTLPPSREEMTCNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 51 | LCDR1 | RSSQSLLSSGNQKNYLT |
| | (Kabat) | |
| SEQ ID NO: 52 | LCDR2 | WASTRES |
| | (Kabat) | |
| SEQ ID NO: 53 | LCDR3 | QNDYSYPLT |
| | (Kabat) | |
| SEQ ID NO: 54 | LCDR1 | SQSLLSSGNQKNY |
| | (Chothia) | |
| SEQ ID NO: 55 | LCDR2 | WAS |
| | (Chothia) | |
| SEQ ID NO: 56 | LCDR3 | DYSYPL |
| | (Chothia) | |
| SEQ ID NO: 57 | VL | DIVMTQTPLSLPVTPGEPASISCRSSQSLLSSGNQKNY |
| | | LTWYLQKPGQSPQLLIYWASTRESGVPDRFSGSGSG |
| | | TDFTLKISRVEAEDVGVYYCQNDYSYPLTFGQGTKLEI |
| | | K |
| SEQ ID NO: 58 | DNA VL | GATATCGTGATGACTCAGACCCCCCTGAGCCTGCC |
| | | CGTGACCCCTGGCGAGCCTGCCTCTATTAGCTGTA |
| | | GATCTAGTCAGTCACTGCTGTCTAGCGGTAATCAGA |
| | | AGAACTACCTGACCTGGTATCTGCAGAAGCCCGGT |
| | | CAGTCACCTCAGCTGCTGATCTACTGGGCCTCTACT |
| | | AGAGAATCAGGCGTGCCCGATAGGTTTAGCGGTAG |
| | | CGGTAGTGGCACCGACTTCACCCTGAAGATCTCTA |
| | | GGGTGGAAGCCGAGGACGTGGGCGTCTACTACTGT |
| | | CAGAACGACTATAGCTACCCCCTGACCTTCGGTCAG |
| | | GGCACTAAGCTGGAGATTAAG |
| SEQ ID NO: 59 | Light Chain | DIVMTQTPLSLPVTPGEPASISCRSSQSLLSSGNQKNY |
| | | LTWYLQKPGQSPQLLIYWASTRESGVPDRFSGSGSG |
| | | TDFTLKISRVEAEDVGVYYCQNDYSYPLTFGQGTKLEI |
| | | KRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |
| SEQ ID NO 60: | DNA Light | GATATCGTGATGACTCAGACCCCCCTGAGCCTGCC |
| | Chain | CGTGACCCCTGGCGAGCCTGCCTCTATTAGCTGTA |
| | | GATCTAGTCAGTCACTGCTGTCTAGCGGTAATCAGA |
| | | AGAACTACCTGACCTGGTATCTGCAGAAGCCCGGT |
| | | CAGTCACCTCAGCTGCTGATCTACTGGGCCTCTACT |
| | | AGAGAATCAGGCGTGCCCGATAGGTTTAGCGGTAG |
| | | CGGTAGTGGCACCGACTTCACCCTGAAGATCTCTA |
| | | GGGTGGAAGCCGAGGACGTGGGCGTCTACTACTGT |
| | | CAGAACGACTATAGCTACCCCCTGACCTTCGGTCAG |
| | | GGCACTAAGCTGGAGATTAAGCGTACGGTGGCCGC |
| | | TCCCAGCGTGTTCATCTTCCCCCCCAGCGACGAGC |
| | | AGCTGAAGAGCGGCACCGCCAGCGTGGTGTGCCT |
| | | GCTGAACAACTTCTACCCCCGGGAGGCCAAGGTGC |
| | | AGTGGAAGGTGGACAACGCCCTGCAGAGCGGCAAC |
| | | AGCCAGGAGAGCGTCACCGAGCAGGACAGCAAGG |
| | | ACTCCACCTACAGCCTGAGCAGCACCCTGACCCTG |
| | | AGCAAGGCCGACTACGAGAAGCATAAGGTGTACGC |
| | | CTGCGAGGTGACCCACCAGGGCCTGTCCAGCCCC |
| | | GTGACCAAGAGCTTCAACAGGGGCGAGTGC |
| SEQ ID NO: 138 | K107C | DIVMTQTPLSLPVTPGEPASISCRSSQSLLSSGNQKNY |
| | CysMab | LTWYLQKPGQSPQLLIYWASTRESGVPDRFSGSGSG |
| | Mutated | TDFTLKISRVEAEDVGVYYCQNDYSYPLTFGQGTKLEI |
| | Light Chain | CRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |

P-Cad Mab4

| | | |
|---|---|---|
| SEQ ID NO: 61 | HCDR1 | DHTLH |
| | (Kabat) | |
| SEQ ID NO: 62 | HCDR2 | YIYPRSGSTKYNENFRG |
| | (Kabat) | |
| SEQ ID NO: 63 | HCDR3 | RLLFLPLDY |
| | (Kabat) | |
| SEQ ID NO: 64 | HCDR1 | GYTFTDH |
| | (Chothia) | |
| SEQ ID NO: 65 | HCDR2 | YPRSGS |
| | (Chothia) | |
| SEQ ID NO: 66 | HCDR3 | RLLFLPLDY |
| | (Chothia) | |
| SEQ ID NO: 67 | VH | QIQLVQSGAEVKKPGSSVKVSCKVSGYTFTDHTLHW |
| | | MRQAPGQGLEWMGYIYPRSGSTKYNENFRGRVTITA |
| | | DTSSSTAYMELSSLRSEDTAVYYCARRLLFLPLDYWG |
| | | QGTLVTVSS |
| SEQ ID NO: 68 | DNA VH | CAGATTCAGCTGGTGCAGTCAGGCGCCGAAGTGAA |
| | | GAAACCCGGCTCTAGCGTGAAAGTCAGCTGTAAAG |
| | | TCTCAGGCTACACCTTCACCGATCACACCCTGCACT |
| | | GGATGAGACAGGCCCCAGGTCAGGGCCTGGAGTG |
| | | GATGGGCTATATCTACCCTAGATCAGGCTCTACTAA |
| | | GTATAACGAGAACTTTAGGGGTAGAGTGACTATCAC |
| | | CGCCGACACTAGCTCTAGCACCGCCTATATGGAACT |
| | | GTCTAGCCTGAGATCAGAGGACACCGCCGTCTACT |
| | | ACTGCGCTAGACGGCTGCTGTTCCTGCCCCTGGAC |
| | | TACTGGGGTCAGGGCACCCTGGTCACCGTGTCTAG |
| | | C |
| SEQ ID NO: 69 | Heavy Chain | QIQLVQSGAEVKKPGSSVKVSCKVSGYTFTDHTLHW |
| | | MRQAPGQGLEWMGYIYPRSGSTKYNENFRGRVTITA |
| | | DTSSSTAYMELSSLRSEDTAVYYCARRLLFLPLDYWG |
| | | QGTLVTVSSASTKGPSVFPLAPSSKSTSGGTAALGCL |
| | | VKDYFPEPVTVSWNSGALTSGVHTFPAVLQSSGLYSL |
| | | SSVVTVPSSSLGTQTYICNVNHKPSNTKVDKRVEPKS |
| | | CDKTHTCPPCPAPELLGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTKNQVSLTC |
| | | LVKGFYPSDIAVEWESNGQPENNYKTTPPVLDSDGSF |
| | | FLYSKLTVDKSRWQQGNVFSCSVMHEALHNHYTQKS |
| | | LSLSPGK |
| SEQ ID NO: 70 | DNA Heavy | CAGATTCAGCTGGTGCAGTCAGGCGCCGAAGTGAA |
| | Chain | GAAACCCGGCTCTAGCGTGAAAGTCAGCTGTAAAG |
| | | TCTCAGGCTACACCTTCACCGATCACACCCTGCACT |
| | | GGATGAGACAGGCCCCAGGTCAGGGCCTGGAGTG |
| | | GATGGGCTATATCTACCCTAGATCAGGCTCTACTAA |
| | | GTATAACGAGAACTTTAGGGGTAGAGTGACTATCAC |
| | | CGCCGACACTAGCTCTAGCACCGCCTATATGGAACT |
| | | GTCTAGCCTGAGATCAGAGGACACCGCCGTCTACT |
| | | ACTGCGCTAGACGGCTGCTGTTCCTGCCCCTGGAC |
| | | TACTGGGGTCAGGGCACCCTGGTCACCGTGTCTAG |
| | | CGCTAGCACTAAGGGCCCAAGTGTGTTTCCCCTGG |
| | | CCCCCAGCAGCAAGTCTACTTCCGGCGGAACTGCT |
| | | GCCCTGGGTTGCCTGGTGAAGGACTACTTCCCCGA |
| | | GCCCGTGACAGTGTCCTGGAACTCTGGGGCTCTGA |
| | | CTTCCGGCGTGCACACCTTCCCCGCCGTGCTGCAG |
| | | AGCAGCGGCCTGTACAGCCTGAGCAGCGTGGTGAC |
| | | AGTGCCCTCCAGCTCTCTGGGAACCCAGACCTATAT |
| | | CTGCAACGTGAACCACAAGCCCAGCAACACCAAGG |
| | | TGGACAAGAGAGTGGAGCCCAAGAGCTGCGACAAG |
| | | ACCCACACCTGCCCCCCCTGCCCAGCTCCAGAACT |
| | | GCTGGGAGGGCCTTCCGTGTTCCTGTTCCCCCCCA |
| | | AGCCCAAGGACACCCTGATGATCAGCAGGACCCCC |
| | | GAGGTGACCTGCGTGGTGGTGGACGTGTCCCACGA |
| | | GGACCCAGAGGTGAAGTTCAACTGGTACGTGGACG |
| | | GCGTGGAGGTGCACAACGCCAAGACCAAGCCCAGA |
| | | GAGGAGCAGTACAACAGCACCTACAGGGTGGTGTC |
| | | CGTGCTGACCGTGCTGCACCAGGACTGGCTGAACG |
| | | GCAAAGAATACAAGTGCAAAGTCTCCAACAAGGCCC |
| | | TGCCAGCCCCAATCGAAAAGACAATCAGCAAGGCC |
| | | AAGGGCCAGCCACGGGAGCCCCAGGTGTACACCCT |
| | | GCCCCCCAGCCGGGAGGAGATGACCAAGAACCAG |
| | | GTGTCCCTGACCTGTCTGGTGAAGGGCTTCTACCC |
| | | CAGCGATATCGCCGTGGAGTGGGAGAGCAACGGC |
| | | CAGCCCGAGAACAACTACAAGACCACCCCCCCAGT |
| | | GCTGGACAGCGACGGCAGCTTCTTCCTGTACAGCA |
| | | AGCTGACCGTGGACAAGTCCAGGTGGCAGCAGGG |
| | | CAACGTGTTCAGCTGCAGCGTGATGCACGAGGCCC |
| | | TGCACAACCACTACACCCAGAAGTCCCTGAGCCTG |
| | | AGCCCCGGCAAG |
| SEQ ID NO: 139 | E152C/S375 | QIQLVQSGAEVKKPGSSVKVSCKVSGYTFTDHTLHW |
| | C CysMab | MRQAPGQGLEWMGYIYPRSGSTKYNENFRGRVTITA |
| | Mutated | DTSSSTAYMELSSLRSEDTAVYYCARRLLFLPLDYWG |
| | Heavy Chain | QGTLVTVSSASTKGPSVFPLAPSSKSTSGGTAALGCL |
| | | VKDYFPCPVTVSWNSGALTSGVHTFPAVLQSSGLYSL |
| | | SSVVTVPSSSLGTQTYICNVNHKPSNTKVDKRVEPKS |
| | | CDKTHTCPPCPAPELLGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTKNQVSLTC |
| | | LVKGFYPCDIAVEWESNGQPENNYKTTPPVLDSDGSF |
| | | FLYSKLTVDKSRWQQGNVFSCSVMHEALHNHYTQKS |
| | | LSLSPGK |
| SEQ ID NO: 140 | K360C | QIQLVQSGAEVKKPGSSVKVSCKVSGYTFTDHTLHW |
| | CysMab | MRQAPGQGLEWMGYIYPRSGSTKYNENFRGRVTITA |
| | Mutated | DTSSSTAYMELSSLRSEDTAVYYCARRLLFLPLDYWG |
| | Heavy Chain | QGTLVTVSSASTKGPSVFPLAPSSKSTSGGTAALGCL |
| | | VKDYFPEPVTVSWNSGALTSGVHTFPAVLQSSGLYSL |
| | | SSVVTVPSSSLGTQTYICNVNHKPSNTKVDKRVEPKS |
| | | CDKTHTCPPCPAPELLGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTCNQVSLTC |
| | | LVKGFYPSDIAVEWESNGQPENNYKTTPPVLDSDGSF |
| | | FLYSKLTVDKSRWQQGNVFSCSVMHEALHNHYTQKS |
| | | LSLSPGK |
| SEQ ID NO: 71 | LCDR1 | RSSQSLLSSGNQKSYLT |
| | (Kabat) | |
| SEQ ID NO: 72 | LCDR2 | WASTRES |
| | (Kabat) | |
| SEQ ID NO: 73 | LCDR3 | QNDYSYPFT |
| | (Kabat) | |
| SEQ ID NO: 74 | LCDR1 | SQSLLSSGNQKSY |
| | (Chothia) | |
| SEQ ID NO: 75 | LCDR2 | WAS |
| | (Chothia) | |
| SEQ ID NO: 76 | LCDR3 | DYSYPF |
| | (Chothia) | |
| SEQ ID NO: 77 | VL | EIVMTQSPATLSLSPGERATLSCRSSQSLLSSGNQKS |
| | | YLTWYQQKPGQAPRLLIYWASTRESGIPARFSGSGSG |
| | | TDFTLTISSLQPEDFAVYYCQNDYSYPFTFGQGTKLEI |
| | | K |
| SEQ ID NO: 78 | DNA VL | GAGATCGTGATGACTCAGTCACCCGCTACCCTGAG |
| | | CCTGAGCCCTGGCGAGAGAGCTACACTGAGCTGTA |
| | | GATCTAGTCAGTCACTGCTGTCTAGCGGTAATCAGA |
| | | AGTCCTACCTGACCTGGTATCAGCAGAAGCCCGGT |
| | | CAGGCCCCTAGACTGCTGATCTACTGGGCCTCTACT |
| | | AGAGAGTCAGGGATCCCCGCTAGGTTTAGCGGTAG |
| | | CGGTAGTGGCACCGACTTCACCCTGACTATCTCTAG |
| | | CCTGCAGCCCGAGGACTTCGCCGTCTACTACTGTC |
| | | AGAACGACTATAGCTACCCCTTCACCTTCGGTCAGG |
| | | GCACTAAGCTGGAGATTAAG |
| SEQ ID NO: 79 | Light Chain | EIVMTQSPATLSLSPGERATLSCRSSQSLLSSGNQKS |
| | | YLTWYQQKPGQAPRLLIYWASTRESGIPARFSGSGSG |
| | | TDFTLTISSLQPEDFAVYYCQNDYSYPFTFGQGTKLEI |
| | | KRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |
| SEQ ID NO 80: | DNA Light | GAGATCGTGATGACTCAGTCACCCGCTACCCTGAG |
| | Chain | CCTGAGCCCTGGCGAGAGAGCTACACTGAGCTGTA |
| | | GATCTAGTCAGTCACTGCTGTCTAGCGGTAATCAGA |
| | | AGTCCTACCTGACCTGGTATCAGCAGAAGCCCGGT |
| | | CAGGCCCCTAGACTGCTGATCTACTGGGCCTCTACT |
| | | AGAGAGTCAGGGATCCCCGCTAGGTTTAGCGGTAG |
| | | CGGTAGTGGCACCGACTTCACCCTGACTATCTCTAG |
| | | CCTGCAGCCCGAGGACTTCGCCGTCTACTACTGTC |
| | | AGAACGACTATAGCTACCCCTTCACCTTCGGTCAGG |
| | | GCACTAAGCTGGAGATTAAGCGTACGGTGGCCGCT |
| | | CCCAGCGTGTTCATCTTCCCCCCCAGCGACGAGCA |
| | | GCTGAAGAGCGGCACCGCCAGCGTGGTGTGCCTG |
| | | CTGAACAACTTCTACCCCCGGGAGGCCAAGGTGCA |
| | | GTGGAAGGTGGACAACGCCCTGCAGAGCGGCAACA |
| | | GCCAGGAGAGCGTCACCGAGCAGGACAGCAAGGA |
| | | CTCCACCTACAGCCTGAGCAGCACCCTGACCCTGA |
| | | GCAAGGCCGACTACGAGAAGCATAAGGTGTACGCC |
| | | TGCGAGGTGACCCACCAGGGCCTGTCCAGCCCCGT |
| | | GACCAAGAGCTTCAACAGGGGCGAGTGC |
| SEQ ID NO: | K107C | EIVMTQSPATLSLSPGERATLSCRSSQSLLSSGNQKS |
| 141 | CysMab | YLTWYQQKPGQAPRLLIYWASTRESGIPARFSGSGSG |
| | Mutated Light | TDFTLTISSLQPEDFAVYYCQNDYSYPFTFGQGTKLEI |
| | Chain | CRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |

P-Cad Mab5

| | | |
|---|---|---|
| SEQ ID NO: 81 | HCDR1 | DHTLH |
| | (Kabat) | |
| SEQ ID NO: 82 | HCDR2 | YIYPRSGSTKYNENFRG |
| | (Kabat) | |
| SEQ ID NO: 83 | HCDR3 | RLLFLPLDY |
| | (Kabat) | |
| SEQ ID NO: 84 | HCDR1 | GYTFTDH |
| | (Chothia) | |
| SEQ ID NO: 85 | HCDR2 | YPRSGS |
| | (Chothia) | |
| SEQ ID NO: 86 | HCDR3 | RLLFLPLDY |
| | (Chothia) | |
| SEQ ID NO: 87 | VH | QIQLVQSGAEVKKPGSSVKVSCKVSGYTFTDHTLHW |
| | | MRQAPGQGLEWMGYIYPRSGSTKYNENFRGRVTITA |
| | | DTSSSTAYMELSSLRSEDTAVYYCVRRLLFLPLDYWG |
| | | QGTLVTVSS |
| SEQ ID NO: 88 | DNA VH | CAGATTCAGCTGGTGCAGTCAGGCGCCGAAGTGAA |
| | | GAAACCCGGCTCTAGCGTGAAAGTCAGCTGTAAAG |
| | | TCTCAGGCTACACCTTCACCGATCACACCCTGCACT |
| | | GGATGAGACAGGCCCCAGGTCAGGGCCTGGAGTG |
| | | GATGGGCTATATCTACCCTAGATCAGGCTCTACTAA |
| | | GTATAACGAGAACTTTAGGGGTAGAGTGACTATCAC |
| | | CGCCGACACTAGCTCTAGCACCGCCTATATGGAACT |
| | | GTCTAGCCTGAGATCAGAGGACACCGCCGTCTACT |
| | | ACTGCGTCAGACGGCTGCTGTTCCTGCCCCTGGAC |
| | | TACTGGGGTCAGGGCACCCTGGTCACCGTGTCTAG |
| | | C |
| SEQ ID NO: 89 | Heavy Chain | QIQLVQSGAEVKKPGSSVKVSCKVSGYTFTDHTLHW |
| | | MRQAPGQGLEWMGYIYPRSGSTKYNENFRGRVTITA |
| | | DTSSSTAYMELSSLRSEDTAVYYCVRRLLFLPLDYWG |
| | | QGTLVTVSSASTKGPSVFPLAPSSKSTSGGTAALGCL |
| | | VKDYFPEPVTVSWNSGALTSGVHTFPAVLQSSGLYSL |
| | | SSVVTVPSSSLGTQTYICNVNHKPSNTKVDKRVEPKS |
| | | CDKTHTCPPCPAPELLGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTKNQVSLTC |
| | | LVKGFYPSDIAVEWESNGQPENNYKTTPPVLDSDGSF |
| | | FLYSKLTVDKSRWQQGNVFSCSVMHEALHNHYTQKS |
| | | LSLSPGK |
| SEQ ID NO: 90 | DNA Heavy | CAGATTCAGCTGGTGCAGTCAGGCGCCGAAGTGAA |
| | Chain | GAAACCCGGCTCTAGCGTGAAAGTCAGCTGTAAAG |
| | | TCTCAGGCTACACCTTCACCGATCACACCCTGCACT |
| | | GGATGAGACAGGCCCCAGGTCAGGGCCTGGAGTG |
| | | GATGGGCTATATCTACCCTAGATCAGGCTCTACTAA |
| | | GTATAACGAGAACTTTAGGGGTAGAGTGACTATCAC |
| | | CGCCGACACTAGCTCTAGCACCGCCTATATGGAACT |
| | | GTCTAGCCTGAGATCAGAGGACACCGCCGTCTACT |
| | | ACTGCGTCAGACGGCTGCTGTTCCTGCCCCTGGAC |
| | | TACTGGGGTCAGGGCACCCTGGTCACCGTGTCTAG |
| | | CGCTAGCACTAAGGGCCCAAGTGTGTTTCCCCTGG |
| | | CCCCCAGCAGCAAGTCTACTTCCGGCGGAACTGCT |
| | | GCCCTGGGTTGCCTGGTGAAGGACTACTTCCCCGA |
| | | GCCCGTGACAGTGTCCTGGAACTCTGGGGCTCTGA |
| | | CTTCCGGCGTGCACACCTTCCCCGCCGTGCTGCAG |
| | | AGCAGCGGCCTGTACAGCCTGAGCAGCGTGGTGAC |
| | | AGTGCCCTCCAGCTCTCTGGGAACCCAGACCTATAT |
| | | CTGCAACGTGAACCACAAGCCCAGCAACACCAAGG |
| | | TGGACAAGAGAGTGGAGCCCAAGAGCTGCGACAAG |
| | | ACCCACACCTGCCCCCCCTGCCCAGCTCCAGAACT |
| | | GCTGGGAGGGCCTTCCGTGTTCCTGTTCCCCCCCA |
| | | AGCCCAAGGACACCCTGATGATCAGCAGGACCCCC |
| | | GAGGTGACCTGCGTGGTGGTGGACGTGTCCCACGA |
| | | GGACCCAGAGGTGAAGTTCAACTGGTACGTGGACG |
| | | GCGTGGAGGTGCACAACGCCAAGACCAAGCCCAGA |
| | | GAGGAGCAGTACAACAGCACCTACAGGGTGGTGTC |
| | | CGTGCTGACCGTGCTGCACCAGGACTGGCTGAACG |
| | | GCAAAGAATACAAGTGCAAAGTCTCCAACAAGGCCC |
| | | TGCCAGCCCCAATCGAAAAGACAATCAGCAAGGCC |
| | | AAGGGCCAGCCACGGGAGCCCCAGGTGTACACCCT |
| | | GCCCCCCAGCCGGGAGGAGATGACCAAGAACCAG |
| | | GTGTCCCTGACCTGTCTGGTGAAGGGCTTCTACCC |
| | | CAGCGATATCGCCGTGGAGTGGGAGAGCAACGGC |
| | | CAGCCCGAGAACAACTACAAGACCACCCCCCCAGT |
| | | GCTGGACAGCGACGGCAGCTTCTTCCTGTACAGCA |
| | | AGCTGACCGTGGACAAGTCCAGGTGGCAGCAGGG |
| | | CAACGTGTTCAGCTGCAGCGTGATGCACGAGGCCC |
| | | TGCACAACCACTACACCCAGAAGTCCCTGAGCCTG |
| | | AGCCCCGGCAAG |
| SEQ ID NO: 142 | E152C/S375 | QIQLVQSGAEVKKPGSSVKVSCKVSGYTFTDHTLHW |
| | C CysMab | MRQAPGQGLEWMGYIYPRSGSTKYNENFRGRVTITA |
| | Mutated | DTSSSTAYMELSSLRSEDTAVYYCVRRLLFLPLDYWG |
| | Heavy Chain | QGTLVTVSSASTKGPSVFPLAPSSKSTSGGTAALGCL |
| | | VKDYFPCPVTVSWNSGALTSGVHTFPAVLQSSGLYSL |
| | | SSVVTVPSSSLGTQTYICNVNHKPSNTKVDKRVEPKS |
| | | CDKTHTCPPCPAPELLGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTKNQVSLTC |
| | | LVKGFYPCDIAVEWESNGQPENNYKTTPPVLDSDGSF |
| | | FLYSKLTVDKSRWQQGNVFSCSVMHEALHNHYTQKS |
| | | LSLSPGK |
| SEQ ID NO: 143 | K360C | QIQLVQSGAEVKKPGSSVKVSCKVSGYTFTDHTLHW |
| | CysMab | MRQAPGQGLEWMGYIYPRSGSTKYNENFRGRVTITA |
| | Mutated | DTSSSTAYMELSSLRSEDTAVYYCVRRLLFLPLDYWG |
| | Heavy Chain | QGTLVTVSSASTKGPSVFPLAPSSKSTSGGTAALGCL |
| | | VKDYFPEPVTVSWNSGALTSGVHTFPAVLQSSGLYSL |
| | | SSVVTVPSSSLGTQTYICNVNHKPSNTKVDKRVEPKS |
| | | CDKTHTCPPCPAPELLGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTCNQVSLTC |
| | | LVKGFYPSDIAVEWESNGQPENNYKTTPPVLDSDGSF |
| | | FLYSKLTVDKSRWQQGNVFSCSVMHEALHNHYTQKS |
| | | LSLSPGK |
| SEQ ID NO: 91 | LCDR1 | RSSQSLLSSGNQKSYLT |
| | (Kabat) | |
| SEQ ID NO: 92 | LCDR2 | WASTRES |
| | (Kabat) | |
| SEQ ID NO: 93 | LCDR3 | QNDYSYPFT |
| | (Kabat) | |
| SEQ ID NO: 94 | LCDR1 | SQSLLSSGNQKSY |
| | (Chothia) | |
| SEQ ID NO: 95 | LCDR2 | WAS |
| | (Chothia) | |
| SEQ ID NO: 96 | LCDR3 | DYSYPF |
| | (Chothia) | |
| SEQ ID NO: 97 | VL | EIVMTQSPATLSLSPGERATLSCRSSQSLLSSGNQKS |
| | | YLTWYQQKPGQAPRLLIYWASTRESGIPARFSGSGSG |
| | | TDFTLTISSLQPEDFAVYYCQNDYSYPFTFGQGTKLEI |
| | | K |
| SEQ ID NO: 98 | DNA VL | GAGATCGTGATGACTCAGTCACCCGCTACCCTGAG |
| | | CCTGAGCCCTGGCGAGAGAGCTACACTGAGCTGTA |
| | | GATCTAGTCAGTCACTGCTGTCTAGCGGTAATCAGA |
| | | AGTCCTACCTGACCTGGTATCAGCAGAAGCCCGGT |
| | | CAGGCCCCTAGACTGCTGATCTACTGGGCCTCTACT |
| | | AGAGAGTCAGGGATCCCCGCTAGGTTTAGCGGTAG |
| | | CGGTAGTGGCACCGACTTCACCCTGACTATCTCTAG |
| | | CCTGCAGCCCGAGGACTTCGCCGTCTACTACTGTC |
| | | AGAACGACTATAGCTACCCCTTCACCTTCGGTCAGG |
| | | GCACTAAGCTGGAGATTAAG |
| SEQ ID NO: 99 | Light Chain | EIVMTQSPATLSLSPGERATLSCRSSQSLLSSGNQKS |
| | | YLTWYQQKPGQAPRLLIYWASTRESGIPARFSGSGSG |
| | | TDFTLTISSLQPEDFAVYYCQNDYSYPFTFGQGTKLEI |
| | | KRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |
| SEQ ID NO: 100 | DNA Light | GAGATCGTGATGACTCAGTCACCCGCTACCCTGAG |
| | Chain | CCTGAGCCCTGGCGAGAGAGCTACACTGAGCTGTA |
| | | GATCTAGTCAGTCACTGCTGTCTAGCGGTAATCAGA |
| | | AGTCCTACCTGACCTGGTATCAGCAGAAGCCCGGT |
| | | CAGGCCCCTAGACTGCTGATCTACTGGGCCTCTACT |
| | | AGAGAGTCAGGGATCCCCGCTAGGTTTAGCGGTAG |
| | | CGGTAGTGGCACCGACTTCACCCTGACTATCTCTAG |
| | | CCTGCAGCCCGAGGACTTCGCCGTCTACTACTGTC |
| | | AGAACGACTATAGCTACCCCTTCACCTTCGGTCAGG |
| | | GCACTAAGCTGGAGATTAAGCGTACGGTGGCCGCT |
| | | CCCAGCGTGTTCATCTTCCCCCCCAGCGACGAGCA |
| | | GCTGAAGAGCGGCACCGCCAGCGTGGTGTGCCTG |
| | | CTGAACAACTTCTACCCCCGGGAGGCCAAGGTGCA |
| | | GTGGAAGGTGGACAACGCCCTGCAGAGCGGCAACA |
| | | GCCAGGAGAGCGTCACCGAGCAGGACAGCAAGGA |
| | | CTCCACCTACAGCCTGAGCAGCACCCTGACCCTGA |
| | | GCAAGGCCGACTACGAGAAGCATAAGGTGTACGCC |
| | | TGCGAGGTGACCCACCAGGGCCTGTCCAGCCCCGT |
| | | GACCAAGAGCTTCAACAGGGGCGAGTGC |
| SEQ ID NO: 144 | K107C | EIVMTQSPATLSLSPGERATLSCRSSQSLLSSGNQKS |
| | CysMab | YLTWYQQKPGQAPRLLIYWASTRESGIPARFSGSGSG |
| | Mutated Light | TDFTLTISSLQPEDFAVYYCQNDYSYPFTFGQGTKLEI |
| | Chain | CRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |

P-Cad Mab6

| | | |
|---|---|---|
| SEQ ID NO: | HCDR1 | DHTLH |
| 101 | (Kabat) | |
| SEQ ID NO: | HCDR2 | YIYPRSGSTKYNENFKG |
| 102 | (Kabat) | |
| SEQ ID NO: | HCDR3 | RLLFLPLDY |
| 103 | (Kabat) | |
| SEQ ID NO: | HCDR1 | GYTFTDH |
| 104 | (Chothia) | |
| SEQ ID NO: | HCDR2 | YPRSGS |
| 105 | (Chothia) | |
| SEQ ID NO: | HCDR3 | RLLFLPLDY |
| 106 | (Chothia) | |
| SEQ ID NO: | VH | QIQLVQSGAEVKKPGSSVKVSCKVSGYTFTDHTLHW |
| 107 | | MRQAPGQGLEWMGYIYPRSGSTKYNENFKGRVTITA |
| | | DTSSSTAYMELSSLRSEDTAVYYCVRRLLFLPLDYWG |
| | | QGTLVTVSS |
| SEQ ID NO: | DNA VH | CAGATTCAGCTGGTGCAGTCAGGCGCCGAAGTGAA |
| 108 | | GAAACCCGGCTCTAGCGTGAAAGTCAGCTGTAAAG |
| | | TCTCAGGCTACACCTTCACCGATCACACCCTGCACT |
| | | GGATGAGACAGGCCCCAGGTCAGGGCCTGGAGTG |
| | | GATGGGCTATATCTACCCTAGATCAGGCTCTACTAA |
| | | GTATAACGAGAACTTTAAGGGTAGAGTGACTATCAC |
| | | CGCCGACACTAGCTCTAGCACCGCCTATATGGAACT |
| | | GTCTAGCCTGAGATCAGAGGACACCGCCGTCTACT |
| | | ACTGCGTCAGACGGCTGCTGTTCCTGCCCCTGGAC |
| | | TACTGGGGTCAGGGCACCCTGGTCACCGTGTCTAG |
| | | C |
| SEQ ID NO: | Heavy Chain | QIQLVQSGAEVKKPGSSVKVSCKVSGYTFTDHTLHW |
| 109 | | MRQAPGQGLEWMGYIYPRSGSTKYNENFKGRVTITA |
| | | DTSSSTAYMELSSLRSEDTAVYYCVRRLLFLPLDYWG |
| | | QGTLVTVSSASTKGPSVFPLAPSSKSTSGGTAALGCL |
| | | VKDYFPEPVTVSWNSGALTSGVHTFPAVLQSSGLYSL |
| | | SSWTVPSSSLGTQTYICNVNHKPSNTKVDKRVEPKS |
| | | CDKTHTCPPCPAPELLGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTKNQVSLTC |
| | | LVKGFYPSDIAVEWESNGQPENNYKTTPPVLDSDGSF |
| | | FLYSKLTVDKSRWQQGNVFSCSVMHEALHNHYTQKS |
| | | LSLSPGK |
| SEQ ID NO: | DNA Heavy | CAGATTCAGCTGGTGCAGTCAGGCGCCGAAGTGAA |
| 110 | Chain | GAAACCCGGCTCTAGCGTGAAAGTCAGCTGTAAAG |
| | | TCTCAGGCTACACCTTCACCGATCACACCCTGCACT |
| | | GGATGAGACAGGCCCCAGGTCAGGGCCTGGAGTG |
| | | GATGGGCTATATCTACCCTAGATCAGGCTCTACTAA |
| | | GTATAACGAGAACTTTAAGGGTAGAGTGACTATCAC |
| | | CGCCGACACTAGCTCTAGCACCGCCTATATGGAACT |
| | | GTCTAGCCTGAGATCAGAGGACACCGCCGTCTACT |
| | | ACTGCGTCAGACGGCTGCTGTTCCTGCCCCTGGAC |
| | | TACTGGGGTCAGGGCACCCTGGTCACCGTGTCTAG |
| | | CGCTAGCACTAAGGGCCCAAGTGTGTTTCCCCTGG |
| | | CCCCCAGCAGCAAGTCTACTTCCGGCGGAACTGCT |
| | | GCCCTGGGTTGCCTGGTGAAGGACTACTTCCCCGA |
| | | GCCCGTGACAGTGTCCTGGAACTCTGGGGCTCTGA |
| | | CTTCCGGCGTGCACACCTTCCCCGCCGTGCTGCAG |
| | | AGCAGCGGCCTGTACAGCCTGAGCAGCGTGGTGAC |
| | | AGTGCCCTCCAGCTCTCTGGGAACCCAGACCTATAT |
| | | CTGCAACGTGAACCACAAGCCCAGCAACACCAAGG |
| | | TGGACAAGAGAGTGGAGCCCAAGAGCTGCGACAAG |
| | | ACCCACACCTGCCCCCCCTGCCCAGCTCCAGAACT |
| | | GCTGGGAGGGCCTTCCGTGTTCCTGTTCCCCCCCA |
| | | AGCCCAAGGACACCCTGATGATCAGCAGGACCCCC |
| | | GAGGTGACCTGCGTGGTGGTGGACGTGTCCCACGA |
| | | GGACCCAGAGGTGAAGTTCAACTGGTACGTGGACG |
| | | GCGTGGAGGTGCACAACGCCAAGACCAAGCCCAGA |
| | | GAGGAGCAGTACAACAGCACCTACAGGGTGGTGTC |
| | | CGTGCTGACCGTGCTGCACCAGGACTGGCTGAACG |
| | | GCAAAGAATACAAGTGCAAAGTCTCCAACAAGGCCC |
| | | TGCCAGCCCCAATCGAAAAGACAATCAGCAAGGCC |
| | | AAGGGCCAGCCACGGGAGCCCCAGGTGTACACCCT |
| | | GCCCCCCAGCCGGGAGGAGATGACCAAGAACCAG |
| | | GTGTCCCTGACCTGTCTGGTGAAGGGCTTCTACCC |
| | | CAGCGATATCGCCGTGGAGTGGGAGAGCAACGGC |
| | | CAGCCCGAGAACAACTACAAGACCACCCCCCCAGT |
| | | GCTGGACAGCGACGGCAGCTTCTTCCTGTACAGCA |
| | | AGCTGACCGTGGACAAGTCCAGGTGGCAGCAGGG |
| | | CAACGTGTTCAGCTGCAGCGTGATGCACGAGGCCC |
| | | TGCACAACCACTACACCCAGAAGTCCCTGAGCCTG |
| | | AGCCCCGGCAAG |
| SEQ ID NO: | E152C/S375 | QIQLVQSGAEVKKPGSSVKVSCKVSGYTFTDHTLHW |
| 145 | C CysMab | MRQAPGQGLEWMGYIYPRSGSTKYNENFKGRVTITA |
| | Mutated | DTSSSTAYMELSSLRSEDTAVYYCVRRLLFLPLDYWG |
| | Heavy Chain | QGTLVTVSSASTKGPSVFPLAPSSKSTSGGTAALGCL |
| | | VKDYFPCPVTVSWNSGALTSGVHTFPAVLQSSGLYSL |
| | | SSWTVPSSSLGTQTYICNVNHKPSNTKVDKRVEPKS |
| | | CDKTHTCPPCPAPELLGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTKNQVSLTC |
| | | LVKGFYPCDIAVEWESNGQPENNYKTTPPVLDSDGSF |
| | | FLYSKLTVDKSRWQQGNVFSCSVMHEALHNHYTQKS |
| | | LSLSPGK |
| SEQ ID NO: | K360C | QIQLVQSGAEVKKPGSSVKVSCKVSGYTFTDHTLHW |
| 146 | CysMab | MRQAPGQGLEWMGYIYPRSGSTKYNENFKGRVTITA |
| | Mutated | DTSSSTAYMELSSLRSEDTAVYYCVRRLLFLPLDYWG |
| | Heavy Chain | QGTLVTVSSASTKGPSVFPLAPSSKSTSGGTAALGCL |
| | | VKDYFPEPVTVSWNSGALTSGVHTFPAVLQSSGLYSL |
| | | SSWTVPSSSLGTQTYICNVNHKPSNTKVDKRVEPKS |
| | | CDKTHTCPPCPAPELLGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTCNQVSLTC |
| | | LVKGFYPSDIAVEWESNGQPENNYKTTPPVLDSDGSF |
| | | FLYSKLTVDKSRWQQGNVFSCSVMHEALHNHYTQKS |
| | | LSLSPGK |
| SEQ ID NO: | LCDR1 | RSSQSLLSSGNQKSYLT |
| 111 | (Kabat) | |
| SEQ ID NO: | LCDR2 | WASTRES |
| 112 | (Kabat) | |
| SEQ ID NO: | LCDR3 | QNDYSYPFT |
| 113 | (Kabat) | |
| SEQ ID NO: | LCDR1 | SQSLLSSGNQKSY |
| 114 | (Chothia) | |
| SEQ ID NO: | LCDR2 | WAS |
| 115 | (Chothia) | |
| SEQ ID NO: | LCDR3 | DYSYPF |
| 116 | (Chothia) | |
| SEQ ID NO: | VL | EIVMTQSPATLSLSPGERATLSCRSSQSLLSSGNQKS |
| 117 | | YLTWYQQKPGQAPRLLIYWASTRESGIPARFSGSGSG |
| | | TDFTLTISSLQPEDFAVYYCQNDYSYPFTFGQGTKLEI |
| | | K |
| SEQ ID NO: | DNA VL | GAGATCGTGATGACTCAGTCACCCGCTACCCTGAG |
| 118 | | CCTGAGCCCTGGCGAGAGAGCTACACTGAGCTGTA |
| | | GATCTAGTCAGTCACTGCTGTCTAGCGGTAATCAGA |
| | | AGTCCTACCTGACCTGGTATCAGCAGAAGCCCGGT |
| | | CAGGCCCCTAGACTGCTGATCTACTGGGCCTCTACT |
| | | AGAGAGTCAGGGATCCCCGCTAGGTTTAGCGGTAG |
| | | CGGTAGTGGCACCGACTTCACCCTGACTATCTCTAG |
| | | CCTGCAGCCCGAGGACTTCGCCGTCTACTACTGTC |
| | | AGAACGACTATAGCTACCCCTTCACCTTCGGTCAGG |
| | | GCACTAAGCTGGAGATTAAG |
| SEQ ID NO: | Light Chain | EIVMTQSPATLSLSPGERATLSCRSSQSLLSSGNQKS |
| 119 | | YLTWYQQKPGQAPRLLIYWASTRESGIPARFSGSGSG |
| | | TDFTLTISSLQPEDFAVYYCQNDYSYPFTFGQGTKLEI |
| | | KRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |
| SEQ ID NO: | DNA Light | GAGATCGTGATGACTCAGTCACCCGCTACCCTGAG |
| 120 | Chain | CCTGAGCCCTGGCGAGAGAGCTACACTGAGCTGTA |
| | | GATCTAGTCAGTCACTGCTGTCTAGCGGTAATCAGA |
| | | AGTCCTACCTGACCTGGTATCAGCAGAAGCCCGGT |
| | | CAGGCCCCTAGACTGCTGATCTACTGGGCCTCTACT |
| | | AGAGAGTCAGGGATCCCCGCTAGGTTTAGCGGTAG |
| | | CGGTAGTGGCACCGACTTCACCCTGACTATCTCTAG |
| | | CCTGCAGCCCGAGGACTTCGCCGTCTACTACTGTC |
| | | AGAACGACTATAGCTACCCCTTCACCTTCGGTCAGG |
| | | GCACTAAGCTGGAGATTAAGCGTACGGTGGCCGCT |
| | | CCCAGCGTGTTCATCTTCCCCCCCAGCGACGAGCA |
| | | GCTGAAGAGCGGCACCGCCAGCGTGGTGTGCCTG |
| | | CTGAACAACTTCTACCCCCGGGAGGCCAAGGTGCA |
| | | GTGGAAGGTGGACAACGCCCTGCAGAGCGGCAACA |
| | | GCCAGGAGAGCGTCACCGAGCAGGACAGCAAGGA |
| | | CTCCACCTACAGCCTGAGCAGCACCCTGACCCTGA |
| | | GCAAGGCCGACTACGAGAAGCATAAGGTGTACGCC |
| | | TGCGAGGTGACCCACCAGGGCCTGTCCAGCCCCGT |
| | | GACCAAGAGCTTCAACAGGGGCGAGTGC |
| SEQ ID NO: | K107C | EIVMTQSPATLSLSPGERATLSCRSSQSLLSSGNQKS |
| 147 | CysMab | YLTWYQQKPGQAPRLLIYWASTRESGIPARFSGSGSG |
| | Mutated Light | TDFTLTISSLQPEDFAVYYCQNDYSYPFTFGQGTKLEI |
| | Chain | CRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |

Cys Mab Mutations to Antibody Constant Regions

| | | |
|---|---|---|
| SEQ ID NO: | E152C/S375 | SASTKGPSVFPLAPSSKSTSGGTAALGCLVKDYFPCP |
| 148 | C Cys Mab | VTVSWNSGALTSGVHTFPAVLQSSGLYSLSSVVTVPS |
| | Mutations to | SSLGTQTYICNVNHKPSNTKVDKKVEPKSCDKTHTCP |
| | Wild Type | PCPAPELLGGPSVFLFPPKPKDTLMISRTPEVTCVVVD |
| | Heavy Chain | VSHEDPEVKFNWYVDGVEVHNAKTKPREEQYNSTYR |
| | Constant | VVSVLTVLHQDWLNGKEYKCKVSNKALPAPIEKTISKA |
| | Region | KGQPREPQVYTLPPSREEMTKNQVSLTCLVKGFYPC |
| | | DIAVEWESNGQPENNYKTTPPVLDSDGSFFLYSKLTV |
| | | DKSRWQQGNVFSCSVMHEALHNHYTQKSLSLSPGK |
| SEQ ID NO: | K360C Cys | SASTKGPSVFPLAPSSKSTSGGTAALGCLVKDYFPEP |
| 149 | Mab | VTVSWNSGALTSGVHTFPAVLQSSGLYSLSSVVTVPS |
| | Mutations to | SSLGTQTYICNVNHKPSNTKVDKKVEPKSCDKTHTCP |
| | Wild Type | PCPAPELLGGPSVFLFPPKPKDTLMISRTPEVTCVVVD |
| | Heavy Chain | VSHEDPEVKFNWYVDGVEVHNAKTKPREEQYNSTYR |
| | Constant | VVSVLTVLHQDWLNGKEYKCKVSNKALPAPIEKTISKA |
| | Region | KGQPREPQVYTLPPSREEMTCNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| SEQ ID NO: | K107C Cys | CRTVAAPSVFIFPPSDEQLKSGTASVVCLLNNFYPREA |
| 150 | Mab | KVQWKVDNALQSGNSQESVTEQDSKDSTYSLSSTLTL |
| | Mutations to | SKADYEKHKVYACEVTHQGLSSPVTKSFNRGEC |
| | Wild Type | |
| | Light Chain | |
| | Constant | |
| | Region | |

Other antibodies of the invention include those where the amino acids or nucleic acids encoding the amino acids have been mutated, yet have at least 60, 70, 80, 90 or 95 percent identity to the sequences described in Table 3. In some embodiments, 1, 2, 3, 4 or 5 amino acids have been mutated in the variable regions when compared with the variable regions depicted in the sequence described in Table 3, while retaining substantially the same therapeutic activity as the antibodies listed in Table 3.

In some embodiments antibodies or antibody fragments (e.g., antigen binding fragment) useful in immunoconjugates of the invention include modified or engineered antibodies, such as an antibody modified to introduce one or more cysteine residues as sites for conjugation to a drug moiety (Junutula J R, et al.: Nat Biotechnol 2008, 26:925-932). In one embodiment, the invention provides a modified antibody or antibody fragment thereof comprising a substitution of one or more amino acids with cysteine at the positions described herein. Sites for cysteine substitution are in the constant regions of the antibody and are thus applicable to a variety of antibodies, and the sites are selected to provide stable and homogeneous conjugates. A modified antibody or fragment can have two or more cysteine substitutions, and these substitutions can be used in combination with other antibody modification and conjugation methods as described herein. Methods for inserting cysteine at specific locations of an antibody are known in the art, see, e.g., Lyons et al, (1990) Protein Eng., 3:703-708, WO 2011/005481, WO2014/124316. In certain embodiments a modified antibody or antibody fragment comprises a substitution of one or more amino acids with cysteine on its constant region selected from positions 117, 119, 121, 124, 139, 152, 153, 155, 157, 164, 169, 171, 174, 189, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain of the antibody or antibody fragment, and wherein the positions are numbered according to the EU system. In some embodiments a modified antibody or antibody fragment comprises a substitution of one or more amino acids with cysteine on its constant region selected from positions 107, 108, 109, 114, 129, 142, 143, 145, 152, 154, 156, 159, 161, 165, 168, 169, 170, 182, 183, 197, 199, and 203 of a light chain of the antibody or antibody fragment, wherein the positions are numbered according to the EU system, and wherein the light chain is a human kappa light chain. In certain embodiments a modified antibody or antibody fragment thereof comprises a combination of substitution of two or more amino acids with cysteine on its constant regions wherein the combinations comprise substitutions at positions 375 of an antibody heavy chain, position 152 of an antibody heavy chain, position 360 of an antibody heavy chain, or position 107 of an antibody light chain and wherein the positions are numbered according to the EU system. In certain embodiments a modified antibody or antibody fragment thereof comprises a substitution of one amino acid with cysteine on its constant regions wherein the substitution is position 375 of an antibody heavy chain, position 152 of an antibody heavy chain, position 360 of an antibody heavy chain, position 107 of an antibody light chain, position 165 of an antibody light chain or position 159 of an antibody light chain and wherein the positions are numbered according to the EU system, and wherein the light chain is a kappa chain. In particular embodiments a modified antibody or antibody fragment thereof comprises a combination of substitution of two amino acids with cysteine on its constant regions wherein the combinations comprise substitutions at positions 375 of an antibody heavy chain and position 152 of an antibody heavy chain, wherein the positions are numbered according to the EU system. In particular embodiments a modified antibody or antibody fragment thereof comprises a substitution of one amino acid with cysteine at position 360 of an antibody heavy chain, wherein the positions are numbered according to the EU system. In other particular embodiments a modified antibody or antibody fragment thereof comprises a substitution of one amino acid with cysteine at position 107 of an antibody light chain and wherein the positions are numbered according to the EU system, and wherein the light chain is a kappa chain. Exemplary embodiments of these positions are illustrated in the constant region sequences disclosed in SEQ ID NOs: 148, 149, and 150. Specific embodiments of these positions are disclosed for the anti-P-cadherin antibody sequences in SEQ ID NOs: 130, 131, 132, 133, 134, 135, 136, 137, 138, 139, 140, 141, 142, 143, 144, 145, 146, and 147.

Since each of these antibodies can bind to P-cadherin, the VH, VL, full length light chain, and full length heavy chain sequences (amino acid sequences and the nucleotide sequences encoding the amino acid sequences) can be “mixed and matched” to create other P-cadherin-binding antibodies of the invention. Such “mixed and matched” P-cadherin-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence. Accordingly, in one aspect, the invention provides antibody conjugates that include an isolated monoclonal antibody or antigen binding region thereof having: a heavy chain variable region comprising an amino acid sequence selected from the group consisting of SEQ ID NOs: 7, 27, 47, 67, 87, and 107; and a light chain variable region comprising an amino acid sequence selected from the group consisting of SEQ ID NOs: 17, 37, 57, 77, 97, and 117; wherein the antibody specifically binds to P-cadherin.

In another aspect, the invention provides antibody conjugates that include (i) an isolated monoclonal antibody having: a full length heavy chain comprising an amino acid sequence that has been optimized for expression in the cell of a mammalian expression system selected from the group consisting of SEQ ID NOs: 9, 29, 49, 69, 89, and 109; and a full length light chain comprising an amino acid sequence that has been optimized for expression in the cell of a mammalian selected from the group consisting of SEQ ID NOs: 19, 39, 59, 79, 99, and 119; or (ii) a functional protein comprising an antigen binding portion thereof.

In another aspect, the present invention provides antibody conjugates that include P-cadherin-binding antibodies that comprise the heavy chain and light chain CDR1s, CDR2s and CDR3s as described in Table 3, or combinations thereof. The amino acid sequences of the VH CDR1s of the antibodies are shown in SEQ ID NOs: 1, 21, 41, 61, 81, and 101. The amino acid sequences of the VH CDR2s of the antibodies and are shown in SEQ ID NOs: 2, 22, 42, 62, 82, and 102. The amino acid sequences of the VH CDR3s of the antibodies are shown in SEQ ID NOs: 3, 23, 43, 63, 83, and 103. The amino acid sequences of the VL CDR1s of the antibodies are shown in SEQ ID NOs: 11, 31, 51, 71, 91, and 111. The amino acid sequences of the VL CDR2s of the antibodies are shown in SEQ ID NOs 12, 32, 52, 72, 92, and 112. The amino acid sequences of the VL CDR3s of the antibodies are shown in SEQ ID NOs: 13, 33, 53, 73, 93, and 113.

Given that each of these antibodies can bind to P-cadherin and that antigen-binding specificity is provided primarily by the CDR1, 2 and 3 regions, the VH CDR1, CDR2 and CDR3 sequences and VL CDR1, CDR2 and CDR3 sequences can be “mixed and matched” (i.e., CDRs from different antibodies can be mixed and matched. Such “mixed and matched” P-cadherin-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present invention.

Accordingly, the present invention provides an isolated monoclonal antibody or antigen binding region thereof comprising a heavy chain CDR1 comprising an amino acid sequence selected from the group consisting of SEQ ID NOs: 1, 21, 41, 61, 81, and 101; a heavy chain CDR2 comprising an amino acid sequence selected from the group consisting of SEQ ID NOs: 2, 22, 42, 62, 82, and 102; a heavy chain CDR3 comprising an amino acid sequence selected from the group consisting of SEQ ID NOs: 3, 23, 43, 63, 83, and 103; a light chain CDR1 comprising an amino acid sequence selected from the group consisting of SEQ ID NOs: 11, 31, 51, 71, 91, and 111; a light chain CDR2 comprising an amino acid sequence selected from the group consisting of SEQ ID NOs: 12, 32, 52, 72, 92, and 112; and a light chain CDR3 comprising an amino acid sequence selected from the group consisting of SEQ ID NOs: 13, 33, 53, 73, 93, and 113; wherein the antibody specifically binds P-cadherin.

In a specific embodiment, an antibody or antibody fragment (e.g., antigen binding fragments) that specifically binds to P-cadherin comprises a heavy chain CDR1 of SEQ ID NO:1, a heavy chain CDR2 of SEQ ID NO: 2; a heavy chain CDR3 of SEQ ID NO:3; a light chain CDR1 of SEQ ID NO:11; a light chain CDR2 of SEQ ID NO: 12; and a light chain CDR3 of SEQ ID NO: 13.

In another specific embodiment, an antibody or antibody fragment (e.g., antigen binding fragments) that specifically binds to P-cadherin comprising a heavy chain CDR1 of SEQ ID NO:21, a heavy chain CDR2 of SEQ ID NO: 22; a heavy chain CDR3 of SEQ ID NO:23; a light chain CDR1 of SEQ ID NO:31; a light chain CDR2 of SEQ ID NO: 32; and a light chain CDR3 of SEQ ID NO: 33.

In a yet another embodiment, an antibody or antibody fragment (e.g., antigen binding fragments) that specifically binds to P-cadherin comprising a heavy chain CDR1 of SEQ ID NO:41, a heavy chain CDR2 of SEQ ID NO: 42; a heavy chain CDR3 of SEQ ID NO:43; a light chain CDR1 of SEQ ID NO:51; a light chain CDR2 of SEQ ID NO: 52; and a light chain CDR3 of SEQ ID NO: 53.

In a further embodiment, an antibody or antibody fragment (e.g., antigen binding fragments) that specifically binds to P-cadherin comprising a heavy chain CDR1 of SEQ ID NO:61, a heavy chain CDR2 of SEQ ID NO: 62; a heavy chain CDR3 of SEQ ID NO:63; a light chain CDR1 of SEQ ID NO:71; a light chain CDR2 of SEQ ID NO: 72; and a light chain CDR3 of SEQ ID NO: 73.

In another specific embodiment, an antibody or antibody fragment (e.g., antigen binding fragments) that specifically binds to P-cadherin comprising a heavy chain CDR1 of SEQ ID NO:81, a heavy chain CDR2 of SEQ ID NO: 82; a heavy chain CDR3 of SEQ ID NO:83; a light chain CDR1 of SEQ ID NO:91; a light chain CDR2 of SEQ ID NO: 92; and a light chain CDR3 of SEQ ID NO: 93.

In a further specific embodiment, an antibody or antibody fragment (e.g., antigen binding fragments) that specifically binds to P-cadherin comprising a heavy chain CDR1 of SEQ ID NO:101, a heavy chain CDR2 of SEQ ID NO: 102; a heavy chain CDR3 of SEQ ID NO:103; a light chain CDR1 of SEQ ID NO:111; a light chain CDR2 of SEQ ID NO: 112; and a light chain CDR3 of SEQ ID NO: 113.

In certain embodiments, an antibody that specifically binds to P-cadherin is an antibody or antibody fragment (e.g., antigen binding fragment) that is described in Table 3.

### 2. Further Alteration of the Framework of Fc Region

The immunoconjugates of the invention may comprise modified antibodies or antigen binding fragments thereof that further comprise modifications to framework residues within VH and/or VL, e.g. to improve the properties of the antibody. In some embodiments, the framework modifications are made to decrease the immunogenicity of the antibody. For example, one approach is to “back-mutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back-mutated” to the germline sequence by, for example, site-directed mutagenesis. Such “back-mutated” antibodies are also intended to be encompassed by the invention.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 20030153043 by Carr et al.

In addition or in the alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in, e.g., U.S. Pat. No. 6,194,551 by Idusogie et al.

In another embodiment, one or more amino acid residues are altered to thereby alter the ability of the antibody to fix complement. This approach is described in, e.g., the PCT Publication WO 94/29351 by Bodmer et al. In a specific embodiment, one or more amino acids of an antibody or antigen binding fragment thereof of the present invention are replaced by one or more allotypic amino acid residues. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the PCT Publication WO 00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001).

In still another embodiment, the glycosylation of an antibody is modified. For example, an aglycosylated antibody can be made (i.e., the antibody lacks glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in, e.g., U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). PCT Publication WO 99/54342 by Umana et al. describes cell lines engineered to express glycoprotein-modifying glycosyl transferases (e.g., beta(1,4)—N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

### 3. Production of Antibodies

Antibodies and antibody fragments (e.g., antigen binding fragments) thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The invention further provides polynucleotides encoding the antibodies described herein, e.g., polynucleotides encoding heavy or light chain variable regions or segments comprising the complementarity determining regions as described herein. In some embodiments, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs: 8, 28, 48, 68, 88, 108, and 151. In some embodiments, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs:18, 38, 58, 78, 98, 118, and 153.

In some embodiments, the polynucleotide encoding the heavy chain has at least 85%, 89%, 90%, 91%, 92%, 93%, 94%, 95%, 96%, 97%, 98%, 99%, or 100% nucleic acid sequence identity with a polynucleotide of SEQ ID NO: 10, 30, 50, 70, 90, 110, or 152. In some embodiments, the polynucleotide encoding the light chain has at least 85%, 89%, 90%, 91%, 92%, 93%, 94%, 95%, 96%, 97%, 98%, 99%, or 100% nucleic acid sequence identity with a polynucleotide of SEQ ID NO: 20, 40, 60, 80, 100, 120, or 154.

The polynucleotides of the invention can encode only the variable region sequence of an antibody. They can also encode both a variable region and a constant region of the antibody. Some of the polynucleotide sequences encode a polypeptide that comprises variable regions of both the heavy chain and the light chain of one of the exemplified anti-P-cadherin antibody. Some other polynucleotides encode two polypeptide segments that respectively are substantially identical to the variable regions of the heavy chain and the light chain of one of the antibodies.

The polynucleotide sequences can be produced by de novo solid-phase DNA synthesis or by PCR mutagenesis of an existing sequence (e.g., sequences as described in the Examples below) encoding an antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the invention are expression vectors and host cells for producing an antibodies described herein. Various expression vectors can be employed to express the polynucleotides encoding the antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of the anti-P-cadherin polynucleotides and polypeptides in mammalian (e.g., human) cells include pThioHis A, B & C, pcDNA™3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an anti-P-cadherin antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of an antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted antibody sequences. More often, the inserted antibody sequences are linked to a signal sequences before inclusion in the vector. Vectors to be used to receive sequences encoding anti-P-cadherin antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the antibody chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express antibody polypeptides of the invention. Insect cells in combination with baculovirus vectors can also be used.

In some preferred embodiments, mammalian host cells are used to express and produce the antibody polypeptides of the present invention. For example, they can be either a hybridoma cell line expressing endogenous immunoglobulin genes (e.g., the myeloma hybridoma clones as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed, including the CHO cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., 2012, MOLECULAR CLONING: A LABORATORY MANUAL, volumes 1-4, Cold Spring Harbor Press, NY). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Therapeutic Uses and Methods of Treatment

Provided antibody conjugates are useful in a variety of applications including, but not limited to, treatment of cancer. In certain embodiments, antibody conjugates provided herein are useful for inhibiting tumor growth, reducing tumor volume, inducing differentiation, and/or reducing the tumorigenicity of a tumor. The methods of use can be in vitro, ex vivo, or in vivo methods.

In some embodiments, provided herein are methods of treating, preventing, or ameliorating a disease, e.g., a cancer, in a subject in need thereof, e.g., a human patient, by administering to the subject any of the antibody conjugates described herein. Also provided is use of the antibody conjugates of the invention to treat or prevent disease in a subject, e.g., a human patient. Additionally provided is use of antibody conjugates in treatment or prevention of disease in a subject. In some embodiments provided are antibody conjugates for use in manufacture of a medicament for treatment or prevention of disease in a subject. In certain embodiments, the disease treated with antibody conjugates is a cancer.

In one aspect, the immunoconjugates described herein can be used to treat a solid tumor. Examples of solid tumors include malignancies, e.g., sarcomas, adenocarcinomas, blastomas, and carcinomas, of the various organ systems, such as those affecting liver, lung, breast, lymphoid, biliarintestinal (e.g., colon), genitourinary tract (e.g., renal, urothelial cells), prostate and pharynx. Adenocarcinomas include malignancies such as most colon cancers, rectal cancer, renal-cell carcinoma, liver cancer, small cell lung cancer, non-small cell carcinoma of the lung, cancer of the small intestine and cancer of the esophagus. In one embodiment, the cancer is a melanoma, e.g., an advanced stage melanoma. Examples of other cancers that can be treated include bone cancer, pancreatic cancer, skin cancer, cancer of the head or neck, cutaneous or intraocular malignant melanoma, uterine cancer, ovarian cancer, rectal cancer, colorectal cancer, cancer of the anal region, cancer of the peritoneum, stomach or gastric cancer, esophageal cancer, salivary gland carcinoma, testicular cancer, uterine cancer, carcinoma of the fallopian tubes, carcinoma of the endometrium, carcinoma of the cervix, carcinoma of the vagina, carcinoma of the vulva, penile carcinoma, glioblastoma, neuroblastoma, cervical cancer, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, neuroendocrine tumors (including carcinoid tumors, gastrinoma, and islet cell cancer), mesothelioma, schwannoma (including acoustic neuroma), meningioma, epidermoid cancer, squamous cell cancer, T-cell lymphoma, environmentally induced cancers including those induced by asbestos, and combinations of said cancers.

In another aspect, the immunoconjugates described herein can be used to treat a hematological cancer. Hematological cancers include leukemia, lymphoma, and malignant lymphoproliferative conditions that affect blood, bone marrow and the lymphatic system.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

In some embodiments, the cancer is a hematologic cancer including but is not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with a tumor antigen expression includes, but not limited to, e.g., atypical and/or non-classical cancers, malignancies, precancerous conditions or proliferative diseases expressing a tumor antigen as described herein. Metastatic lesions of the aforementioned cancers can also be treated or prevented using the methods and compositions of the invention.

In certain embodiments, the cancer is characterized by cells expressing a target tumor antigen to which the antibodies, or antibody fragments (e.g., antigen binding fragments) of the antibody conjugates bind. In some embodiments, an immunoconjugate as described herein can comprise an antigen binding domain (e.g., antibody or antibody fragment) that binds to a tumor antigen (e.g., a tumor antigen as described herein). Methods of detecting the presence or overexpression of such tumor antigens are known to persons skilled in the art, and include methods such as immunohistocompatibility (IHC) assays using antibodies that specifically bind the tumor antigens, detecting the level of RNA expression of the tumor antigen, etc.

In some embodiments, the tumor antigen is selected from one or more of the following targets: receptor tyrosine-protein kinase ERBB2 (Her2/neu); receptor tyrosine-protein kinase ERBB3 (Her3); receptor tyrosine-protein kinase ERBB4 (Her4); epidermal growth factor receptor (EGFR); E-cadherin; P-cadherin; Cadherin 6; cathepsin D; estrogen receptor; progesterone receptor; CA125; CA15-3; CA19-9; P-glycoprotein (CD243); CD2; CD19; CD20; CD22; CD24; CD27; CD30; CD37; CD38; CD40; CD44v6; CD45; CD47; CD52; CD56; CD70; CD71; CD79a; CD79b; CD72; CD97; CD179a; CD123; CD137; CD171; CS-1 (also referred to as CD2 subset 1, CRACC, SLAMF7, CD319, and 19A24); C-type lectin-like molecule-1 (CLL-1 or CLECL1); epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11 Ra); prostate stem cell antigen (PSCA); Protease Serine 21 (Testisin or PRSS21); vascular endothelial growth factor receptor 2 (VEGFR2); Lewis(Y) antigen; Platelet-derived growth factor receptor beta (PDGFR-beta); Stage-specific embryonic antigen-4 (SSEA-4); Folate receptor alpha; neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); thyroid stimulating hormone receptor (TSHR); G protein-coupled receptor class C group 5, member D (GPRC5D); chromosome X open reading frame 61 (CXORF61); anaplastic lymphoma kinase (ALK); Polysialic acid; placenta-specific 1 (PLAC1); hexasaccharide portion of globoH glycoceramide (GloboH); mammary gland differentiation antigen (NY-BR-1); adrenoceptor beta 3 (ADRB3); pannexin 3 (PANX3); G protein-coupled receptor 20 (GPR20); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); and immunoglobulin lambda-like polypeptide 1 (IGLL1); CD184; LGR5; AXL; RON; CD352/SLAMf6; KAAG-1; 5T4; c-Met; ITGA3; Endosialin; CD166; SAIL (c15orf54); NaPi2b; DLL3; CD133; FZD7; Dysadherin; PD-L1; SLITRK6; Nectin-4; FGFR2; FGFR3; FGFR4; CEACAM1; CEACAM5; CD74; STEAP-1; PMEL17; Muc16; FcRH5; TENB2; Ly6E; ETBR; 158P1D7; 161P2F10B; 191p4d12; 162p1e6; Notch3; PTK7; and EFNA4.

Tumor-Supporting Antigens

In some embodiments, an immunoconjugate as described herein can comprise an antigen binding domain (e.g., antibody or antibody fragment) that binds to a tumor-supporting antigen (e.g., a tumor-supporting antigen as described herein).

In some embodiments, the tumor-supporting antigen is an antigen present on a stromal cell, an antigen presenting cell, or a myeloid-derived suppressor cell (MDSC). Stromal cells can secrete growth factors to promote cell division in the microenvironment. MDSC cells can inhibit T cell proliferation and activation. In some embodiments, the stromal cell antigen is chosen from one or more of: bone marrow stromal cell antigen 2 (BST2), fibroblast activation protein (FAP) and tenascin. In embodiments, the MDSC antigen is chosen from one or more of: CD33, CD11b, C14, CD15, and CD66b. Accordingly, in some embodiments, the tumor-supporting antigen is chosen from one or more of: bone marrow stromal cell antigen 2 (BST2), fibroblast activation protein (FAP) or tenascin, CD33, CD11b, C14, CD15, and CD66b.

It is also contemplated that the antibody conjugates described herein may be used to treat various non-malignant diseases or disorders, such as inflammatory bowel disease (IBD), gastrointestinal ulcers, Menetrier's disease, hepatitis B, hepatitis C, secreting adenomas or protein loss syndrome, renal disorders, angiogenic disorders, ocular disease such as age related macular degeneration, presumed ocular histoplasmosis syndrome, or age related macular degeneration, bone associated pathologies such as osteoarthritis, rickets and osteoporosis, hyperviscosity syndrome systemic, Osler Weber-Rendu disease, chronic occlusive pulmonary disease, or edema following burns, trauma, radiation, stroke, hypoxia or ischemia, diabetic nephropathy, Paget's disease, photoaging (e.g., caused by UV radiation of human skin), benign prostatic hypertrophy, certain microbial infections including microbial pathogens selected from adenovirus, hantaviruses, Borrelia burgdorferi, Yersinia spp., and Bordetella pertussis, thrombus caused by platelet aggregation, reproductive conditions such as endometriosis, ovarian hyperstimulation syndrome, preeclampsia, dysfunctional uterine bleeding, or menometrorrhagia, acute and chronic nephropathies (including proliferative glomerulonephritis), hypertrophic scar formation, endotoxic shock and fungal infection, familial adenomatosis polyposis, myelodysplastic syndromes, aplastic anemia, ischemic injury, fibrosis of the lung, kidney or liver, infantile hypertrophic pyloric stenosis, urinary obstructive syndrome, psoriatic arthritis.

Method of administration of such antibody conjugates include, but are not limited to, parenteral (e.g., intravenous) administration, e.g., injection as a bolus or continuous infusion over a period of time, oral administration, intramuscular administration, intratumoral administration, intramuscular administration, intraperitoneal administration, intracerobrospinal administration, subcutaneous administration, intra-articular administration, intrasynovial administration, injection to lymph nodes, or intrathecal administration.

For treatment of disease, appropriate dosage of antibody conjugates of the present invention depends on various factors, such as the type of disease to be treated, the severity and course of the disease, the responsiveness of the disease, previous therapy, patient's clinical history, and so on. Antibody conjugates can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of a particular antibody conjugate. In some embodiments, dosage is from 0.01 mg to 20 mg (e.g., 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg) per kg of body weight, and can be given once or more daily, weekly, monthly or yearly. In certain embodiments, the antibody conjugate of the present invention is given once every two weeks or once every three weeks. In certain embodiments, the antibody conjugate of the present invention is given only once. The treating physician can estimate repetition rates for dosing based on measured residence times and concentrations of the drug in bodily fluids or tissues.

### Pharmaceutical Compositions

To prepare pharmaceutical or sterile compositions including one or more antibody conjugates described herein, provided antibody conjugate can be mixed with a pharmaceutically acceptable carrier or excipient.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, N Y, 1993; Lieberman, et al. (eds.), Pharmaceutical Dosage Forms: Tablets, Marcel Dekker, N Y, 1990; Lieberman, et al. (eds.) Pharmaceutical Dosage Forms: Disperse Systems, Marcel Dekker, N Y, 1990; Weiner and Kotkoskie, Excipient Toxicity and Safety, Marcel Dekker, Inc., New York, N.Y., 2000).

In some embodiments, the pharmaceutical composition comprising the antibody conjugate of the present invention is a lyophilisate preparation. In certain embodiments a pharmaceutical composition comprising the antibody conjugate is a lyophilisate in a vial containing an antibody conjugate, histidine, sucrose, and polysorbate 20. In certain embodiments the pharmaceutical composition comprising the antibody conjugate is a lyophilisate in a vial containing an antibody conjugate, sodium succinate, and polysorbate 20. In certain embodiments the pharmaceutical composition comprising the antibody conjugate is a lyophilisate in a vial containing an antibody conjugate, trehalose, citrate, and polysorbate 8. The lyophilisate can be reconstituted, e.g., with water, saline, for injection. In a specific embodiment, the solution comprises the antibody conjugate, histidine, sucrose, and polysorbate 20 at a pH of about 5.0. In another specific embodiment the solution comprises the antibody conjugate, sodium succinate, and polysorbate 20. In another specific embodiment, the solution comprises the antibody conjugate, trehalose dehydrate, citrate dehydrate, citric acid, and polysorbate 8 at a pH of about 6.6. For intravenous administration, the obtained solution will usually be further diluted into a carrier solution.

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak, Antibody Therapy, Bios Scientific Pub. Ltd, Oxfordshire, U K, 1996; Kresina (ed.), Monoclonal Antibodies, Cytokines and Arthritis, Marcel Dekker, New York, N.Y., 1991; Bach (ed.), Monoclonal Antibodies and Peptide Therapy in Autoimmune Diseases, Marcel Dekker, New York, N.Y., 1993; Baert et al., New Engl. J. Med. 348:601-608, 2003; Milgrom et al., New Engl. J. Med. 341:1966-1973, 1999; Slamon et al., New Engl. J. Med. 344:783-792, 2001; Beniaminovitz et al., New Engl. J. Med. 342:613-619, 2000; Ghosh et al., New Engl. J. Med. 348:24-32, 2003; Lipsky et al., New Engl. J. Med. 343:1594-1602, 2000).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Compositions comprising the antibody conjugate of the invention can be provided by continuous infusion, or by doses at intervals of, e.g., one day, one week, or 1-7 times per week, once every other week, once every three weeks, once every four weeks, once every five weeks, once every six weeks, once every seven weeks, or once very eight weeks. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects.

For the antibody conjugates of the invention, the dosage administered to a patient may be 0.0001 mg/kg to 100 mg/kg of the patient's body weight. The dosage may be between 0.001 mg/kg and 50 mg/kg, 0.005 mg/kg and 20 mg/kg, 0.01 mg/kg and 20 mg/kg, 0.02 mg/kg and 10 mg/kg, 0.05 and 5 mg/kg, 0.1 mg/kg and 10 mg/kg, 0.1 mg/kg and 8 mg/kg, 0.1 mg/kg and 5 mg/kg, 0.1 mg/kg and 2 mg/kg, 0.1 mg/kg and 1 mg/kg of the patient's body weight. The dosage of the antibody conjugate may be calculated using the patient's weight in kilograms (kg) multiplied by the dose to be administered in mg/kg.

Doses of the antibody conjugates the invention may be repeated and the administrations may be separated by less than 1 day, at least 1 day, 2 days, 3 days, 5 days, 10 days, 15 days, 30 days, 45 days, 2 months, 75 days, 3 months, 4 months, 5 months, or at least 6 months. In some embodiments, an antibody conjugate of the invention is administered twice weekly, once weekly, once every two weeks, once every three weeks, once every four weeks, or less frequently. In a specific embodiment, doses of the antibody conjugates of the invention are repeated every 2 weeks.

An effective amount for a particular patient may vary depending on factors such as the condition being treated, the overall health of the patient, the method, route and dose of administration and the severity of side effects (see, e.g., Maynard et al., A Handbook of SOPs for Good Clinical Practice, Interpharm Press, Boca Raton, Fla., 1996; Dent, Good Laboratory and Good Clinical Practice, Urch Publ., London, U K, 2001).

The route of administration may be by, e.g., topical or cutaneous application, injection or infusion by subcutaneous, intravenous, intraperitoneal, intracerebral, intramuscular, intraocular, intraarterial, intracerebrospinal, intralesional administration, or by sustained release systems or an implant (see, e.g., Sidman et al., Biopolymers 22:547-556, 1983; Langer et al., J. Biomed. Mater. Res. 15:167-277, 1981; Langer, Chem. Tech. 12:98-105, 1982; Epstein et al., Proc. Natl. Acad. Sci. USA 82:3688-3692, 1985; Hwang et al., Proc. Natl. Acad. Sci. USA 77:4030-4034, 1980; U.S. Pat. Nos. 6,350,466 and 6,316,024). Where necessary, the composition may also include a solubilizing agent or a local anesthetic such as lidocaine to ease pain at the site of the injection, or both. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

Examples of such additional ingredients are well-known in the art.

Methods for co-administration or treatment with a second therapeutic agent, e.g., a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the antibody conjugates of the invention may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibody conjugates of the invention. The two or more therapies may be administered within one same patient visit.

In certain embodiments, the antibody conjugates of the invention can be formulated to ensure proper distribution in vivo. Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (Bloeman et al., (1995) FEBS Lett. 357:140; Owais et al., (1995) Antimicrob. Agents Chemother. 39:180); surfactant Protein A receptor (Briscoe et al., (1995) Am. J. Physiol. 1233:134); p 120 (Schreier et al, (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The invention provides protocols for the administration of pharmaceutical composition comprising antibody conjugates of the invention alone or in combination with other therapies to a subject in need thereof. The therapies (e.g., prophylactic or therapeutic agents) of the combination therapies of the present invention can be administered concomitantly or sequentially to a subject. The therapy (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the invention can be administered to a subject concurrently.

The term “concurrently” is not limited to the administration of therapies (e.g., prophylactic or therapeutic agents) at exactly the same time, but rather it is meant that a pharmaceutical composition comprising antibodies or fragments thereof the invention are administered to a subject in a sequence and within a time interval such that the antibodies or antibody conjugates of the invention can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 5 minutes apart, less than 15 minutes apart, less than 30 minutes apart, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies (e.g., prophylactic or therapeutic agents) are administered within the same patient visit.

Prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

### EXAMPLES

The invention is further described in the following examples, which are not intended to limit the scope of the invention described in the claims.

Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art or can be produced by organic synthesis methods as described herein.

### Abbreviations

Abbreviations used are those conventional in the art or the following:

| | |
|---|---|
| Ac: Acetyl | d: doublet; dd: doublet of doublets |
| AcOH, HOAc: acetic acid | HPLC: high pressure liquid chromatography |
| ACN: Acetonitrile | h, hr: hour(s) |
| AIBN: Azobisisobutyronitrile | HRMS: high resolution mass spectrometry |
| DCM: dichloromethane | Isco, ISCO: Flash chromatography cartridge |
| | containing silica gel provided by Teledyne Isco |
| DMF: N,N-dimethylformamide | LC and LCMS: liquid chromatography and liquid |
| | chromatography-mass spectrometry |
| DMSO: dimethylsulfoxide | Lit: litre |
| DIEA or DIPEA: N,N- | μL, mL and L: microliter, milliliter and liter |
| diisopropylethylamine | |
| Et and EtOAc: ethyl and ethyl acetate | m: multiplet |
| EtOH: ethanol | mg: milligram |
| Fmoc: Fluorenylmethyloxycarbonyl | Min(s): minute(s) |
| chloride | |
| Fmoc-OSU: 9- | M and mM: molar and millimolar |
| Fluorenylmethoxycarbonyl-N- | |
| Succinimide Ester | |
| MeOH: methanol | m/z: mass to charge ratio |
| NBS: N-Bromosuccinimide | NMR: nuclear magnetic resonance |
| TBDPS: tert-Butyldiphenylsilyl | rt: room temperature |
| tBuOH: tert-butanol | RP: Reverse phase |
| TFA: trifluoroacetic acid | s: singlet |
| THF: tetrahydrofuran | t: triplet |
| | TLC: Thin layer chromatography |

### Analytical Methods

LC/MS Data was Acquired Using an Instrument with the Following Parameters:

| | |
|---|---|
| Pump | Waters AcQuity UPLC Binary Solvent Manager |
| Sample Manager | Waters AcQuity UPLC Sample Manager |
| Column Compartment | Waters AcQuity UPLC Column Manager |
| Detector | Waters AcQuity UPLC PDA |
| ELSD | Shimadzu ELSD-LTII |
| Mass Spec | Waters SQD |
| Columns | AcQuity UPLC BEH C18 1.7 μm 2.1 × 50 mm |
| Eluent A1 | 0.1% Formic Acid in Water |
| Eluent B1 | 0.1% Formic Acid in Acetonitrile |
| Eluent A2 | 5 mM Ammonium Hydroxide in Water |
| Eluent B2 | 5 mM Ammonium Hydroxide in Acetonitrile |

The methods used to generate LC/MS data were as follows:
Method A: 2 min acidic method

Eluent A1: 0.1% Formic Acid in Water

Eluent B1: 0.1% Formic Acid in Acetonitrile

Flow: 1.0 mL/min

Stop Time: 3.00 min

pH: 2.6

Column: AcQuity UPLC BEH C18 1.7 μm 2.1×50 mm

Column Temperature: 50° C.

TAC: 210-400 nm

Mass Range: 120-1500 Da

Scan Time: 0.3 sec

Gradient:

| Time | % A (Eluent A1) | % B (Eluent B1) |
|---|---|---|
| 0.00 | 95 | 5 |
| 0.20 | 95 | 5 |
| 2.00 | 5 | 95 |
| 2.50 | 5 | 95 |
| 2.60 | 95 | 5 |
| 3.00 | 95 | 5 |

Method B: 2 min basic method

Eluent A2: 5 mM Ammonium Hydroxide in Water

Eluent B2: 5 mM Ammonium Hydroxide in Acetonitrile

Flow: 1.0 mL/min

Stop Time: 3.00 min

pH: 10.2

Column: AcQuity UPLC BEH C18 1.7 μm 2.1×50 mm

Column Temperature: 50° C.

TAC: 210-400 nm

Mass Range: 120-1500 Da

Scan Time: 0.3 sec

Gradient:

| Time | % A (Eluent A2) | % B (Eluent B2) |
|---|---|---|
| 0.00 | 95 | 5 |
| 0.20 | 95 | 5 |
| 2.00 | 5 | 95 |
| 2.50 | 5 | 95 |
| 2.60 | 95 | 5 |
| 3.00 | 95 | 5 |

Method C: 5 min acidic method

Flow: 1.0 mL/min

Stop Time: 5.20 min

pH: 2.6

Column: AcQuity UPLC BEH C18 1.7 μm 2.1×50 mm

Column Temperature: 50° C.

TAC: 210-400 nm

Mass Range: 120-1500 Da

Scan Time: 0.3 sec

Gradient:

| Time | % A (Eluent A1) | % B (Eluent B1) |
|---|---|---|
| 0.00 | 98 | 2 |
| 4.40 | 2 | 98 |
| 5.15 | 2 | 98 |
| 5.19 | 98 | 2 |

HRMS Data was Acquired Using an Instrument with the Following Parameters:

| | |
|---|---|
| Pump | Waters AcQuity UPLC Binary Solvent Manager |
| Sample Manager | Waters AcQuity UPLC Sample Manager |
| Column Compartment | Waters AcQuity UPLC Column Manager |
| Detector | Waters AcQuity UPLC PDA |
| ELSD | n/a |
| Mass Spec | Waters Xevo G2 Qtof |
| Columns | AcQuity UPLC PrST C4 300 Å 1.7 μm 2.1 × 100 mm |
| | AcQuity UPLC CSH C18 1.7 um 2.1 × 50 mm |
| | ProSwift RP-3U 4.6 × 50 mm SS |
| Eluent A1 | 0.1% Formic Acid in Water |
| Eluent B1 | 0.1% Formic Acid in Acetonitrile |
| Eluent A2 | 0.05% Trifluoroacetic Acid in Water |
| Eluent B2 | 0.05% Trifluoroacetic Acid in Acetonitrile |

The method used to generate HRMS data for linker/payloads and synthetic intermediates was as follows:
Method D: 5 min acidic method

Flow: 1.0 mL/min

Stop Time: 5.2 min

pH: 2.6

Column: AcQuity UPLC BEH C18 1.7 μm 2.1×50 mm

Column Temperature: 50° C.

TAC: 210-400 nm

Mass Range: 300-4000 Da

Processing Range: n/a

Scan Time: 0.5 sec

Gradient:

| Time | % A (Eluent A2) | % B (Eluent B2) |
|---|---|---|
| 0.00 | 98 | 2 |
| 4.40 | 2 | 98 |
| 5.15 | 2 | 98 |
| 5.19 | 98 | 2 |

The method used to generate HRMS data for antibody-drug conjugates was as follows:
Method E: Protein method

Flow: 1.0 mL/min

Stop Time: 3.30 min

pH: 2.6

Column: ProSwift RP-3U 4.6×50 mm SS

Column Temperature: 50° C.

TAC: 210-400 nm

Mass Range: 600-3900 Da

Processing Range: 14000-170000 Da

Scan Time: 1.5 sec

Gradient:

| Time | % A (Eluent A1) | % B (Eluent B1) |
|---|---|---|
| 0.00 | 98 | 2 |
| 0.70 | 98 | 2 |
| 2.00 | 2 | 98 |
| 2.10 | 2 | 98 |
| 2.30 | 98 | 2 |
| 3.30 | 98 | 2 |

Size Exclusion Chromatography data was acquired using an instrument with the following parameters and a run length of 12 minutes:

| | |
|---|---|
| Pump | Waters bioAcQuity UPLC Quaternary Solvent Manager |
| Sample Manager | Waters bioAcQuity UPLC Sample Manager FTN |
| Column Compartment | Waters AcQuity UPLC 30 cm Column Heater |
| Detector | Waters AcQuity UPLC PDA |
| ELSD | n/a |
| Mass Spec | n/a |
| Columns | Superdex 200 Increase 5/150 GL |
| Eluent A1 | 1X PBS (Phosphate Buffered Saline) + 0.1M NaCl + |
| | 5% Isopropanol |
| Eluent B1 | n/a |

### Example 1: Synthesis of Linker Intermediates

### Example 1-1: Synthesis of tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (LI-1)

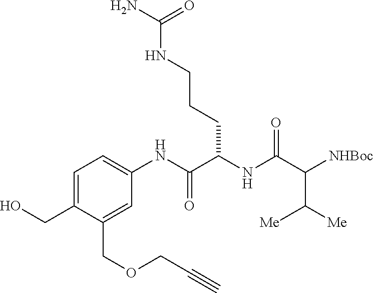 [SMILES: C#CCOCc1cc(NC(=O)[C@H](CCCNC(N)=O)NC(=O)C(NC(=O)OC(C)(C)C)C(C)C)ccc1CO]

### Step 1: Synthesis of 2-(bromomethyl)-4-nitrobenzoic acid

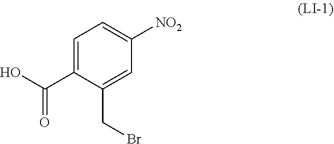 [SMILES: O=C(O)c1ccc([N+](=O)[O-])cc1CBr]

To a stirred solution of 2-methyl-4-nitrobenzoic acid (300 g, 1.5371 mol) in CCl₄ (3000 mL) was added NBS (300.93 g, 1.6908 mol) and AIBN (37.86 g, 0.2305 mol) at rt. The reaction mixture was stirred at 80° C. for 16 h. Reaction mixture was monitored by TLC analysis. The reaction mixture was diluted with sat. NaHCO₃ solution (2 lit) and extracted with ethyl acetate (2×2 lit). The combined organic layer was dried over anhydrous sodium sulphate and concentrated under reduced pressure. The crude compound was purified by column chromatography on silica gel using 2-3% of ethylacetate in petroleum ether as an eluent and 2-(bromomethyl)-4-nitrobenzoic acid was obtained (250 g, 59% yield). ¹H NMR (400 MHz, CDCl3): δ 8.35 (d, J=2.0 Hz, 1H), 8.20 (q, J=8.8, 2.4 Hz, 1H), 8.12 (d, J=8.8 Hz, 1H), 4.97 (s, 2H), 4.00 (s, 3H).

### Step 2: Synthesis of 4-nitro-2-((prop-2-yn-1-yloxy)methyl)benzoic acid

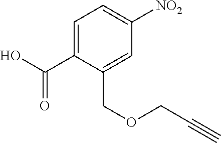 [SMILES: C#CCOCc1cc([N+](=O)[O-])ccc1C(=O)O]

To the mixture of 2-(bromomethyl)-4-nitrobenzoic acid (250 g, 0.9122 mol) in ACN (5000 mL) was added prop-2-yn-1-ol (255.68 g, 265.50 mL, 4.5609 mol, d=0.963 g/mL) and Cs₂CO₃ (743.03 g, 2.2805 mol) at rt. The resulting mixture was heated to 80° C. for 16 h. The reaction mixture was filtered through celite pad washed with ethylacetate (2 lit). The filterate was concentrated under reduced pressure. The obtained crude compound was added sat. NaHCO₃ solution (1 lit) and the aq layer was acidified to pH 2 by using 2N HCl (2 lit). After filteration vacuum drying 4-nitro-2-((prop-2-yn-1-yloxy)methyl)benzoic acid was obtained (130 g, 60.6%). ¹H NMR (400 MHz, DMSO): δ 13.61 (brs, 1H), 8.37 (d, J=2.4 Hz, 1H), 8.23 (dd, J=2.4, 8.4 Hz, 1H), 8.10 (d, J=8.8 Hz, 1H), 4.95 (s, 2H), 4.37 (d, J=2.4 Hz, 2H), 3.52 (t, J=2.4 Hz, 1H)

### Step 3: Synthesis of methyl 4-nitro-2-((prop-2-yn-1-yloxy)methyl)benzoate

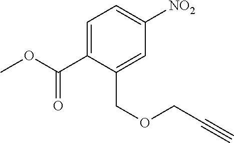 [SMILES: C#CCOCc1cc([N+](=O)[O-])ccc1C(=O)OC]

To a stirred solution of 4-nitro-2-((prop-2-yn-1-yloxy)methyl)benzoic acid (130 g, 0.5527 mol) in MeOH (1300 mL) was added SOCl₂ (526.08 g, 320.78 mL, 4.4219 mol, d=1.64 g/mL) slowly at 0° C. The reaction stirred at 70° C. for 4 h. The reaction solvent was evaporated under reduced pressure. The obtained residue was dissolved in ethylacetate (1000 mL) and washed with sat.NaHCO₃ (600 mL), water (500 mL) and brine solution (500 mL). The separated organic layer was dried over sodium sulphate, filtered and evaporated under reduced pressure to yield methyl 4-nitro-2-((prop-2-yn-1-yloxy)methyl)benzoate (110 g, 80% yield). ¹H NMR (400 MHz, CDCl3): δ 8.56 (t, J=0.8 Hz, 1H), 8.18-8.09 (m, 2H), 5.03 (s, 2H), 4.35 (d, J=2.4 Hz, 2H), 3.96 (s, 3H), 2.49 (t, J=2.4 Hz, 1H).

### Step 4: Synthesis of methyl 4-amino-2-((prop-2-yn-1-yloxy)methyl)benzoate

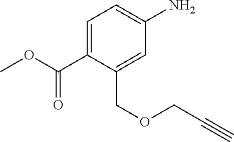 [SMILES: C#CCOCc1cc(N)ccc1C(=O)OC]

To a solution of methyl 4-nitro-2-((prop-2-yn-1-yloxy)methyl)benzoate (110 g, 0.4414 mol) in a mixture of EtOH (1100 mL) and H₂O (550 mL) was added Fe Powder (197.21 g, 3.5310 mol) and NH₄Cl (188.88 g, 3.5310 mol) at rt. The resulting mixture was heated at 80° C. for 16 h. The reaction mixture was cooled to rt and filtered through celite and washed with ethylacetate (2 lit). The filtrate was concentrated under reduced pressure up to half of the volume. To the residue ethylacetate (1.5 lit) was added and separated the two layers and the aqueous layer was extracted with ethyl acetate (2 lit). The combined organic layer was dried over anhydrous sodium sulphate and concentrated under reduced pressure to obtain crude product. Purification by SiO₂ column chromatography (15-20% of ethylacetate in pet-ether) yielded methyl 4-amino-2-((prop-2-yn-1-yloxy)methyl)benzoate (70 g, 72% yield). ¹H NMR (400 MHz, CDCl3): δ 7.67 (d, J=8.8 Hz, 1H), 6.78 (t, J=1.6 Hz, 1H), 6.48 (q, J=8.4, 2.4 Hz, 1H), 4.79 (s, 2H), 4.25 (d, J=2.4 Hz, 2H), 3.70 (d, J=4.0 Hz, 3H), 3.42 (t, J=2.4 Hz, 1H).

### Step 5: Synthesis of (4-amino-2-((prop-2-yn-1-yloxy)methyl)phenyl)methanol

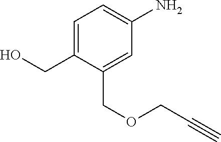

To a stirred solution of THF (1000 mL) was added LiAlH₄ (1 M in THF) (21.23 g, 798.2 mmol, 798.2 mL) slowly at 0° C. A solution of methyl 4-amino-2-((prop-2-yn-1-yloxy)methyl)benzoate (70 g, 319.3 mmol) in THF (800 mL) was added slowly at 0° C. The reaction was stirred at rt for 4 h. The reaction mixture was cooled to 0° C., then was added water (22 mL) very slowly and followed by the addition of 20% NaOH (22 mL) and water (66 mL). The reaction mixture was stirred at 0° C. for 30 min. Anhydrous sodium sulphate was added to absorb excess of water. The mixture was filtered through celite. The filter cake was washed with ethylacetate (1000 mL) and 10% MeOH/DCM (500 mL). The filtrate was concentrated under reduced pressure. The resulting crude compound was purified by SiO₂ column chromotography (35-40% of ethylacetate in pet-ether as an eluent) to give yield (4-amino-2-((prop-2-yn-1-yloxy)methyl)phenyl)methanol (50.6 g, 83% yield). ¹H NMR (400 MHz, CDCl3): δ 6.98 (d, J=8.0 Hz, 1H), 6.56 (d, J=2.4 Hz, 1H), 6.43 (dd, J=2.4, 8.0 Hz, 1H), 4.98 (s, 2H), 4.64 (t, J=5.2 Hz, 1H), 4.47 (s, 2H), 4.34 (d, J=5.6 Hz, 2H), 4.15 (d, J=2.4 Hz, 2H), 3.46 (t, J=2.4 Hz, 1H).

### Step 6: Synthesis of (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate

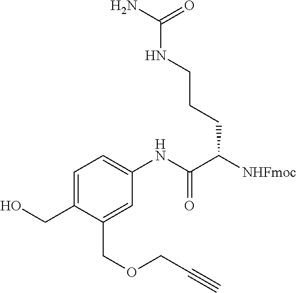

To a solution of (4-amino-2-((prop-2-yn-1-yloxy)methyl)phenyl)methanol (1.92 g, 10.04 mmoles, 1.0 equiv.), (9H-fluoren-9-yl)methyl (S)-(1-amino-1-oxo-5-ureidopentan-2-yl)carbamate (3.99 g, 10.04 mmoles, 1.0 equiv.), and (1-[bis(dimethylamino)methylene]-1H-1,2,3-triazolo[4,5-b]pyridinium 3-oxid hexafluorophosphate (4.20 g, 11.04 mmoles, 1.1 equiv.) in DMF (10 mL) was added N,N-diisopropylethylamine (2.62 mL, 15.06 mmoles, 1.5 equiv.). After stirring at ambient temperature for 1 hour, the mixture was poured into water (200 mL). The resulting solids were filtered, rinsed with water, and dried under vacuum, and (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate was obtained (6.08 g, 99%). LCMS: MH+=571.5; Rt=0.93 min (2 min acidic method-Method A).

### Step 7: Synthesis of (S)-2-amino-N-(4-(hydroxymethyl)-3-((prop-2-yn-1-yloxy)methyl)phenyl)-5-ureidopentanamide

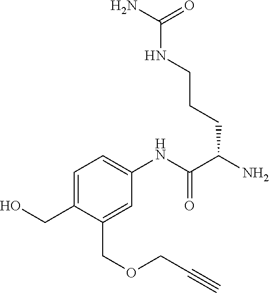 [SMILES: C#CCOCc1cc(NC(=O)[C@@H](N)CCCNC(N)=O)ccc1CO]

To (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate (6.08 g, 10.65 mmoles, 1.0 equiv.) was added dimethylamine (2 M in THF, 21.31 mL, 42.62 mmoles, 4 equiv.). After stirring at ambient temperature for 1.5 hours, the supernatant solution was decanted from the gumlike residue that had formed. The residue was triturated with ether (3×50 mL) and the resulting solids were filtered, washed with ether, and dried under vacuum. (S)-2-amino-N-(4-(hydroxymethyl)-3-((prop-2-yn-1-yloxy)methyl)phenyl)-5-ureidopentanamide was obtained (3.50 g, 10.04 mmoles, 94%). LCMS: MH+349.3; Rt=0.42 min (2 min acidic method-Method A).

### Step 8: Synthesis of tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (LI-1)

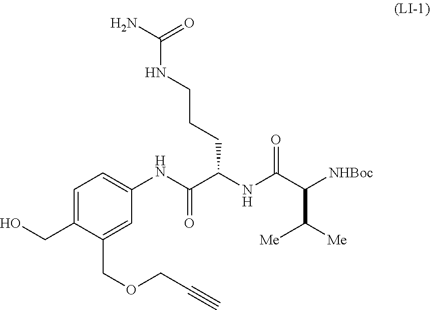 [SMILES: C#CCOCc1cc(NC(=O)[C@H](CCCNC(N)=O)NC(=O)[C@@H](NC(=O)OC(C)(C)C)C(C)C)ccc1CO]

To a solution of (S)-2-amino-N-(4-(hydroxymethyl)-3-((prop-2-yn-1-yloxy)methyl)phenyl)-5-ureidopentanamide (3.50 g, 10.04 mmoles, 1.0 equiv.), (tert-butoxycarbonyl)-L-valine (2.62 g, 12.05 mmol, 1.2 equiv.), and (1-[bis(dimethylamino)methylene]-1H-1,2,3-triazolo[4,5-b]pyridinium 3-oxid hexafluorophosphate (4.58 g, 12.05 mmoles, 1.2 equiv.) in DMF (10 mL) was added N,N-diisopropylethylamine (3.50 mL, 20.08 mmoles, 2.0 equiv). After stirring at ambient temperature for 2 hours, the mixture was poured into water (200 mL) and the resulting suspension was extracted with EtOAc (3×100 mL). The combined organic layers were dried over sodium sulfate and concentrated under vacuum. After purification by ISCO SiO₂ chromatography (0-20% methanol/dichloromethane), tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (LI-1) was obtained (2.49 g, 4.55 mmoles, 45%). 1H NMR (400 MHz, DMSO-d6) δ 10.00 (s, 1H), 7.96 (d, J=7.7 Hz, 1H), 7.55 (dq, J=4.9, 2.2 Hz, 2H, aryl), 7.32 (d, J=8.9 Hz, 1H, aryl), 6.76 (d, J=8.9 Hz, 1H), 5.95 (t, J=5.8 Hz, 1H), 5.38 (s, 2H), 5.01 (t, J=5.5 Hz, 1H), 4.54 (s, 2H), 4.45 (dd, J=25.2, 5.3 Hz, 3H), 4.20 (d, J=2.4 Hz, 2H), 3.83 (dd, J=8.9, 6.7 Hz, 1H), 3.49 (t, J=2.4 Hz, 1H), 2.97 (dh, J=26.0, 6.5 Hz, 2H), 1.96 (h, J=6.6 Hz, 1H), 1.74-1.50 (m, 2H), 1.39 (m, 11H), 0.84 (dd, J=16.2, 6.7 Hz, 6H). LCMS: MNa+570.5; Rt=0.79 min (2 min acidic method-Method A).

### Example 1-2: Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate (LI-2)

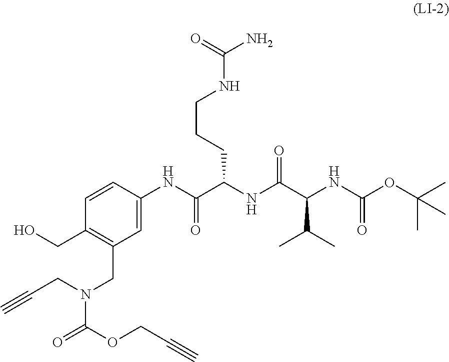 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1CO]

### Step 1: Synthesis of 2-(((tert-butyldiphenylsilyl)oxy)methyl)-5-nitrobenzoic acid

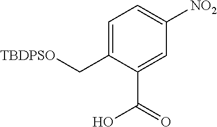

To a solution of 6-nitroisobenzofuran-1(3H)-one (90 g, 502.43 mmol, 1.00 eq) in MeOH (1000 mL) and KOH (28.19 g, 502.43 mmol, 1.00 eq) in H2O (150 mL) was added. The brown mixture was stirred at 25° C. for 1.5 h. The brown mixture was concentrated under reduced pressure to give a residue and dissolved in DCM (2000 mL). The mixture was added TBDPSCl (296.91 g, 1.08 mol, 277.49 mL, 2.15 eq) and imidazole (171.03 g, 2.51 mol, 5.00 eq) stirred at 25° C. for 12 h. The mixture was concentrated under reduced pressure to give a residue. The residue was purified by silica gel chromatography (Petroleum ether/Ethyl acetate=I/O, 1/1) and 2-(((tert-butyldiphenylsilyl)oxy)methyl)-5-nitrobenzoic acid (34 g, 74.16 mmol, 14.76% yield) was obtained as a white solid. ¹H NMR (400 MHz, METHANOL-d4) δ ppm 1.13 (s, 9H) 5.26 (s, 2H) 7.34-7.48 (m, 6H) 7.68 (br d, J=8 Hz, 4H) 8.24 (br d, J=8 Hz, 1H) 8.46 (br d, J=8 Hz, 1H) 8.74 (s, 1H)

### Step 2: Synthesis of (2-(((tert-butyldiphenylsilyl)oxy)methyl)-5-nitrophenyl)methanol

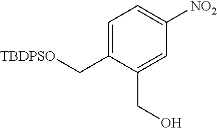

To a mixture of 2-(((tert-butyldiphenylsilyl)oxy)methyl)-5-nitrobenzoic acid (41 g, 94.14 mmol, 1 eq) in THF (205 mL) was added BH₃. THF (1 M, 470.68 mL, 5 eq). The yellow mixture was stirred at 60° C. for 2 h. The mixture was added MeOH (400 mL), and concentrated under reduced pressure to give a residue. then addition of H₂O (200 mL) and DCM (300 mL), extracted with DCM (3×200 mL), washed with brine (300 mL), dried over anhydrous MgSO₄, filtered, and concentrated under reduced pressure to give a residue. The residue was purified by silica gel chromatography (Petroleum ether/Ethyl acetate=1/0, 1/1). (2-(((tert-butyldiphenylsilyl)oxy)methyl)-5-nitrophenyl)methanol (34 g, 80.65 mmol, 85.7% yield) was obtained as a white solid.

1H NMR (400 MHz, METHANOL-d4) δ ppm 1.10 (s, 9H) 4.58 (s, 2H) 4.89 (s, 2H) 7.32-7.51 (m, 6H) 7.68 (dd, J=8, 1.38 Hz, 4H) 7.76 (d, J=8 Hz, 1H) 8.15 (dd, J=8 2.26 Hz, 1H) 8.30 (d, J=2 Hz, 1H).

### Step 3: Synthesis of 2-(((tert-butyldiphenylsilyl)oxy)methyl)-5-nitrobenzaldehyde

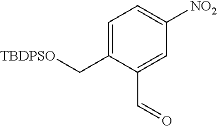

To a solution of (2-(((tert-butyldiphenylsilyloxy)methyl)-5-nitrophenyl)methanol (34 g, 80.65 mmol, 1 eq) in DCM (450 mL) was added MnO2 (56.09 g, 645.22 mmol, 8 eq). The black mixture was stirred at 25° C. for 36 h. The mixture was added MeOH (400 mL), and concentrated under reduced pressure to give a residue. then addition of H₂O (200 mL) and DCM (300 mL), extracted with DCM (3×200 mL), washed with brine (300 mL), dried over anhydrous MgSO₄, filtered, and concentrated under reduced pressure to give a residue. The residue was purified by silica gel chromatography (CH₂Cl₂=100%). 2-(((tert-butyldiphenylsilyl)oxy)methyl)-5-nitrobenzaldehyde (30 g, 71.51 mmol, 88.7% yield) was obtained as a white solid.

¹H NMR (400 MHz, CHLOROFORM-d) δ ppm 1.14 (s, 9H) 5.26 (s, 2H) 7.34-7.53 (m, 6H) 7.60-7.73 (m, 4H) 8.13 (d, J=8 Hz, 1H) 8.48 (dd, J=8, 2.51 Hz, 1H) 8.67 (d, J=2 Hz, 1H) 10.16 (s, 1H)

### Step 4: Synthesis of N-(2-(((tert-butyldiphenylsilyl)oxy)methyl)-5-nitrobenzyl)prop-2-yn-1-amine

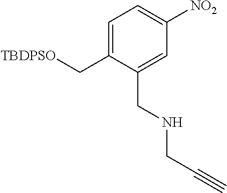

To a solution of 2-(((tert-butyldiphenylsilyl)oxy)methyl)-5-nitrobenzaldehyde (12.6 g, 30.03 mmol, 1 eq) in DCM (130 mL) was added prop-2-yn-1-amine (4.14 g, 75.08 mmol, 4.81 mL, 2.5 eq) and MgSO₄ (36.15 g, 300.33 mmol, 10 eq) then the suspension mixture was stirred at 25° C. for 24 hr. Take a little reaction solution and treat with NaBH₄ the TLC showed one new point was formed. The reaction mixture was filtered and concentrated under reduced pressure to give a residue. (E)-N-[[2-[[tert-butyl(diphenyl)silyl]oxymethyl]-5-nitro-phenyl]methyl]prop-2-yn-1-imine (12 g, crude) was obtained as a yellow solid. ¹H NMR (400 MHz, CHLOROFORM-d) δ ppm 1.11 (s, 9H) 2.48 (t, J=2.38 Hz, 1H) 4.52 (t, J=2.13 Hz, 2H) 5.09 (s, 2H) 7.35-7.49 (m, 6H) 7.63-7.72 (m, 4H) 7.79 (d, J=8.53 Hz, 1H) 8.25 (dd, J=8.53, 2.51 Hz, 1H) 8.68 (d, J=2.26 Hz, 1H) 8.84 (t, J=1.88 Hz, 1H).

(E)-N-[[2-[[tert-butyl(diphenyl)silyl]oxymethyl]-5-nitro-phenyl]methyl]prop-2-yn-1-imine (12 g, 26.28 mmol, 1 eq) was dissolved in MeOH (100 mL) and THF (50 mL), then NaBH₄ (1.49 g, 39.42 mmol, 1.5 eq) was added and the yellow mixture was stirred at −20° C. for 2 hr. LCMS showed that the desired compound was detected. The reaction mixture was quenched by addition MeOH 200 mL at −20° C., and then concentrated under reduced pressure to give a residue. The residue was dissolved with EtOAc 500 mL washed with brine 150 mL, dried over anhydrous Na₂SO₄, filtered and concentrated under reduced pressure to give a residue. The residue was purified by flash silica gel chromatography (Eluent of 0-10% Ethyl acetate/Petroleum ether gradient). N-(2-(((tert-butyldiphenylsilyl)oxy)methyl)-5-nitrobenzyl)prop-2-yn-1-amine (9 g, 18.45 mmol, 70% yield) was obtained as a pale yellow oil. ¹H NMR (400 MHz, CHLOROFORM-d) δ ppm 1.12 (s, 9H) 2.13 (t, J=2.38 Hz, 1H) 3.33 (d, J=2.51 Hz, 2H) 3.80 (s, 2H) 4.93 (s, 2H) 7.36-7.49 (m, 6H) 7.69 (dd, J=7.91, 1.38 Hz, 4H) 7.77 (d, J=8.53 Hz, 1H) 8.16 (dd, J=8.41, 2.38 Hz, 1H) 8.24 (d, J=2.26 Hz, 1H).

### Step 5: Synthesis of (9H-fluoren-9-yl)methyl (2-(((tert-butyldiphenylsilyl)oxy)methyl)-5-nitrobenzyl)(prop-2-yn-1-yl)carbamate

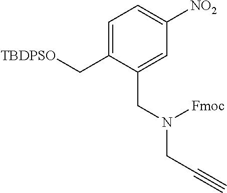

A solution of N-(2-(((tert-butyldiphenylsilyl)oxy)methyl)-5-nitrobenzyl)prop-2-yn-1-amine (9 g, 19.62 mmol, 1 eq) and Fmoc-OSU (7.28 g, 21.59 mmol, 1.1 eq) in dioxane (90 mL) was added sat. NaHCO₃ (90 mL) and the white suspension was stirred at 20° C. for 12 hr. The reaction mixture was diluted with H₂O 150 mL and extracted twice with EtOAc (150 mL each time). The combined organic layers were washed with brine 200 mL, dried over anhydrous Na₂SO₄, filtered and concentrated under reduced pressure to give a residue. The residue was purified by flash silica gel chromatography (Eluent of 0-30% Ethyl acetate/Petroleum ether). (9H-fluoren-9-yl)methyl (2-(((tert-butyldiphenylsilyl)oxy)methyl)-5-nitrobenzyl)(prop-2-yn-1-yl)carbamate (7.7 g, 11.08 mmol, 56.48% yield, 98% purity) was obtained as a white solid. 1H NMR (400 MHz, CHLOROFORM-d) δ ppm 1.12 (s, 9H) 2.17 (br d, J=14.31 Hz, 1H) 3.87-4.97 (m, 9H) 6.98-8.28 (m, 21H).

### Step 6: Synthesis of (9H-fluoren-9-yl)methyl (5-amino-2-(((tert-butyldiphenylsilyl)oxy)methyl)benzyl)(prop-2-yn-1-yl)carbamate

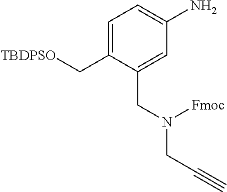

To an ice bath cooled solution of (9H-fluoren-9-yl)methyl (2-(((tert-butyldiphenylsilyl)oxy)methyl)-5-nitrobenzyl)(prop-2-yn-1-yl)carbamate (5.0 g, 7.34 mmoles, 1.0 equiv.) in 10% AcOH/CH₂Cl₂ (100 mL) was added Zn (7.20 g, 110 mmoles, 15 equiv.). The ice bath was removed and the resulting mixture stirred for 2 hours at which time it was filtered through a pad of celite. The volatiles were removed in vacuo and the residue was dissolved in EtOAc, was washed with NaHCO₃ (sat.), NaCl (sat.), dried over MgSO₄, filtered, concentrated and after ISCO SiO₂ chromatography (0-75% EtOAc/Heptanes) (9H-fluoren-9-yl)methyl (5-amino-2-(((tert-butyldiphenylsilyl)oxy)methyl)benzyl)(prop-2-yn-1-yl)carbamate was obtained (2.99 g, 62%). LCMS: MH+=651.6; Rt=3.77 min (5 min acidic method-Method C).

### Step 7: Synthesis of (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate

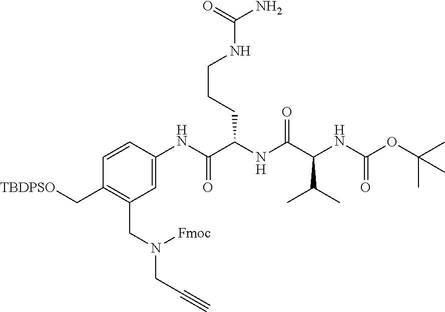

To (9H-fluoren-9-yl)methyl (5-amino-2-(((tert-butyldiphenylsilyl)oxy)methyl)benzyl)(prop-2-yn-1-yl)carbamate (2.99 g, 4.59 mmoles, 1.0 equiv) and (S)-2-((S)-2-((tert-butoxycarbonyl)amino)-3-methylbutanamido)-5-ureidopentanoic acid (1.72 g, 4.59 mmoles, 1.0 equiv.) in CH₂Cl₂ (40 mL) was added ethyl 2-ethoxyquinoline-1(2H)-carboxylate (2.27 g, 9.18 mmoles, 2.0 equiv.). After stirring for 10 minutes, MeOH (1 mL) was added and the solution became homogeneous. The reaction was stirred for 16 hours, the volatiles were removed in vacuo and after purification by ISCO SiO₂ chromatography (0-15% MeOH/CH₂Cl₂) (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate was obtained (2.78 g, 60%). LCMS: MH+=1008.8; Rt=3.77 min (5 min acidic method-Method C).

### Step 8: Synthesis of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate

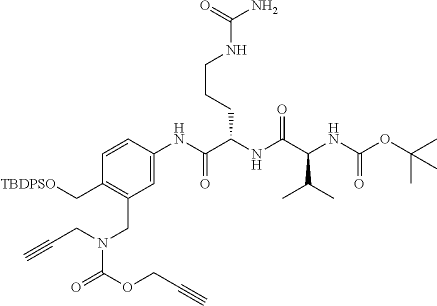

To (9H-fluoren-9-yl)methy (5-((S)-2-((S)-2-(tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate (1.60 g, 1.588 mmoles, 1.0 equiv.) was added 2M dimethylamine in MeOH (30 mL, 60 mmol, 37 equiv.) and THF (10 mL). After standing for 3 hours the volatiles were removed in vacuo and the residue was triturated with Et₂O to remove Fmoc deprotection byproducts. To the resulting solid was added CH₂Cl₂ (16 mL) and pyridine (4 mL) and to the heterogeneous solution was added propargyl chloroformate (155 uL, 1.588 mmole, 1.0 equiv.). After stirring for 30 minutes additional propargyl chloroformate (155 uL, 1.588 mmole, 1.0 equiv.) was added. After stirring for an additional 20 minutes MeOH (1 mL) was added to quench the remaining chloroformate and the volatiles were removed in vacuo. Upon purification by ISCO SiO₂ chromatography (0-15% MeOH/CH₂Cl₂) prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate was obtained (984 mg, 71%). LCMS: MH+=867.8; Rt=3.40 min (5 min acidic method-Method C).

### Step 9: Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate (LI-2)

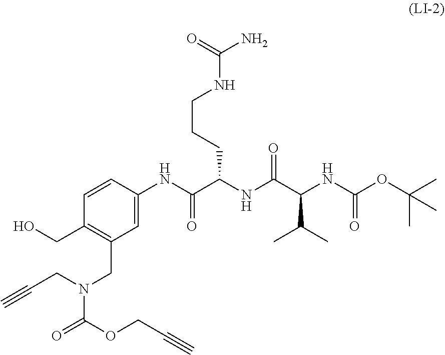 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1CO]

To a solution of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate (984 mg, 1.135 mmoles, 1.0 equiv.) in THF (7.5 mL) was added 1.0 M tetrabutylammoniumn fluoride in THF (2.27 mL, 2.27 mmoles, 2.0 equiv.). After standing for 6 hours the volatiles were removed in vacuo, the residue was purified by ISCO SiO₂ chromatography (0-40% MeOH/CH₂Cl₂) and prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate was obtained (629 mg, 88%). LCMS: MH+=629.6; Rt=1.74 min (5 min acidic method-Method C).

### Example 1-3: Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate (LI-3)

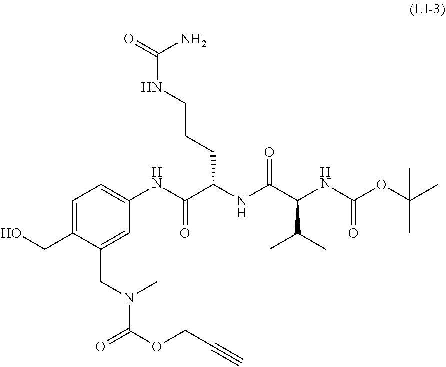 [SMILES: C#CCOC(=O)N(C)Cc1cc(NC(=O)[C@H](CCCNC(N)=O)NC(=O)[C@@H](NC(=O)OC(C)(C)C)C(C)C)ccc1CO]

### Step 1: Synthesis of 2-(hydroxymethyl)-N-methyl-5-nitrobenzamide

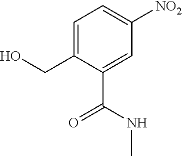

To a stirred suspension of 6-nitroisobenzofuran-1(3H)-one (500 g, 2.79 mol) in MeOH (1500 mL) was added MeNH₂ (3.00 kg, 29.94 mol, 600 mL, 31.0% purity) at 25° C. and stirred for 1 h. The solid was filtered and washed with water twice (600 mL) and dried under high vacuum to get a residue. The product 2-(hydroxymethyl)-N-methyl-5-nitrobenzamide (560 g, crude) was obtained as white solid. LCMS: RT=0.537 min, MS m/z=193.2. 1H NMR: 400 MHz DMSO δ 8.57 (br d, J=4.4 Hz, 1H), 8.31 (dd, J=2.4, 8.6 Hz, 1H), 8.21 (d, J=2.4 Hz, 1H), 7.86 (d, J=8.8 Hz, 1H), 5.54 (t, J=5.6 Hz, 1H), 4.72 (d, J=5.5 Hz, 2H), 2.78 (d, J=4.4 Hz, 3H).

### Step 2: Synthesis of (2-((methylamino)methyl)-4-nitrophenyl)methanol

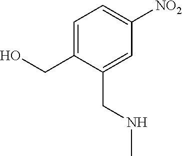

A solution of 2-(hydroxymethyl)-N-methyl-5-nitrobenzamide (560 g, 2.66 mol) in THF (5000 mL) was cooled to 0° C., then BH₃-Me₂S (506 g, 6.66 mol) (2.0 M in THF) was added drop wise for 60 min and the mixture was heated to 70° C. for 5 h. LCMS showed the starting material was consumed. After completion, 4M HCl (1200 mL) in Methanol was added to the reaction mixture at 0° C. and heated at 65° C. for 8 h. The reaction mixture was cooled to 0° C., the solid was filtered and concentrated in reduce pressure. (2-((methylamino)methyl)-4-nitrophenyl)methanol was obtained as a white solid (520 g). LCMS: RT=0.742 min, MS m/z=197.1 [M+H]+. ¹H NMR: 400 MHz DMSO δ 9.25 (br s, 2H), 8.37 (d, J=2.4 Hz, 1H), 8.14 (dd, J=2.4, 8.5 Hz, 1H), 7.63 (d, J=8.4 Hz, 1H), 5.72 (br s, 1H), 4.65 (s, 2H), 4.15 (br s, 2H), 2.55-2.45 (m, 3H)

### Step 3: Synthesis of 1-(2-(((tert-butyldiphenylsilyl)oxy)methyl)-5-nitrophenyl)-N-methylmethanamine

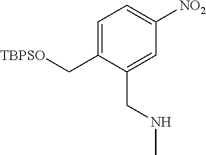

A solution of (2-((methylamino)methyl)-4-nitrophenyl)methanol (520 g, 2.65 mol) and imidazole (721 g, 10.6 mol) in DCM (2600 mL) was cooled to 0° C. then TBDPS-Cl (1.09 kg, 3.98 mol, 1.02 L) was added drop wise and the mixture was stirred for 2 h. The mixture was poured into ice cold water (1000 mL) and extracted with ethyl acetate. The combined organic layers were washed with brine, dried over Na₂SO₄, filtered and evaporated under vacuum to give a crude product. The crude product was purified by chromatography on a silica gel eluted with ethyl acetate:Petroleum ether (from 10/1 to 1) to give a residue. 1-(2-(((tert-butyldiphenylsilyl)oxy)methyl)-5-nitrophenyl)-N-methylmethanamine (600 g) was obtained as a yellow liquid. LCMS: product: RT=0.910 min, MS m/z=435.2 [M+H]+

1H NMR: 400 MHz CDCl3 δ 8.23 (d, J=2.4 Hz, 1H), 8.15 (dd, J=2.4, 8.4 Hz, 1H), 7.76 (d, J=8.4 Hz, 1H), 7.71-7.66 (m, 4H), 7.50-7.37 (m, 6H), 4.88 (s, 2H), 3.65 (s, 2H), 2.39 (s, 3H), 1.12 (s, 9H)

### Step 4: Synthesis of (9H-fluoren-9-yl)methyl (2-(((tert-butyldiphenylsilyl)oxy)methyl)-5-nitrobenzyl)(methyl)carbamate

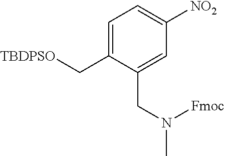

To a solution of 1-(2-(((tert-butyldiphenylsilyl)oxy)methyl)-5-nitrophenyl)-N-methylmethanamine (400 g, 920.3 mmol) in THF (4000 mL) was added Fmoc-OSU (341.5 g, 1.01 mol) and Et₃N (186.2 g, 1.84 mol, 256.2 mL), and the mixture was stirred at 25° C. for 1 h. The mixture was poured into water (1600 mL) and extracted twice with ethyl acetate (1000 mL). The combined organic layers were washed with brine, dried over Na₂SO₄, filtered and evaporated under vacuum to give crude product. The crude product was purified by chromatography on a silica gel eluted with petroleum ether:ethyl acetate (from 1/0 to 1/1) to give (9H-fluoren-9-yl)methyl (2-(((tert-butyldiphenylsilyl)oxy)methyl)-5-nitrobenzyl)(methyl)carbamate (405 g) as a white solid. LCMS: RT=0.931 min, MS m/z=657.2 [M+H]+.

1H NMR: 400 MHz CDCl3 δ 8.21-7.96 (m, 1H), 7.87-7.68 (m, 3H), 7.68-7.62 (m, 4H), 7.62-7.47 (m, 2H), 7.47-7.28 (m, 9H), 7.26-7.05 (m, 2H), 4.81 (br s, 1H), 4.62-4.37 (m, 4H), 4.31-4.19 (m, 1H), 4.08-3.95 (m, 1H), 2.87 (br d, J=5.2 Hz, 3H), 1.12 (s, 9H).

### Step 5: Synthesis of (9H-fluoren-9-yl)methyl (5-amino-2-(((tert-butyldiphenylsilyl)oxy)methyl)benzyl)(methyl)carbamate

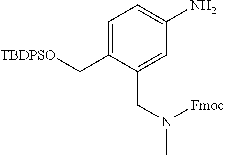

A solution of (9H-fluoren-9-yl)methyl (2-(((tert-butyldiphenylsilyl)oxy)methyl)-5-nitrobenzyl)(methyl)carbamate (3.0 g, 4.57 mmole, 1.0 equiv.) in MeOH (90 mL) and EtOAc (30 mL) was degassed and purged to a balloon of N₂ via three way stopcock. After repeating degas/N₂ purge 2×, 10% Pd/C deGussa type (0.486 g, 0.457 mmoles, 0.1 equiv.) was added. The resulting mixture was degassed and purged to a balloon of 2H₂ via three way stopcock. After repeating degas/H₂ purge 2×, the reaction stirred under the balloon pressure of H₂ for 4 hours. The reaction was degassed and purged to N₂, filtered through a pad of celite eluting further with MeOH. After removal of the volatiles in vacuo and pumping on high vac (9H-fluoren-9-yl)methyl (5-amino-2-(((tert-butyldiphenylsilyl)oxy)methyl)benzyl)(methyl)carbamate was obtained (2.78 g, 97%). LCMS: MH+=627.7; Rt=1.59 min (2 min acidic method-Method A). ¹H NMR: 400 MHz CDCl3 δ 7.80 (br d, J=7.2 Hz, 1H), 7.74-7.67 (m, 5H), 7.64 (br d, J=6.8 Hz, 1H), 7.49-7.30 (m, 10H), 7.23-7.06 (m, 2H), 6.61-6.41 (m, 2H), 4.66 (br d, J=7.2 Hz, 2H), 4.55 (s, 2H), 4.51-4.34 (m, 2H), 4.32-4.10 (m, 1H), 3.66 (br s, 2H), 2.96-2.78 (m, 3H), , 1.07 (s, 9H).

### Step 6: Synthesis of (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate

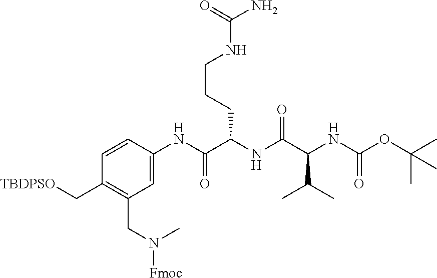

To (9H-fluoren-9-yl)methyl (5-amino-2-(((tert-butyldiphenylsilyl)oxy)methyl)benzyl)(methyl)carbamate (2.86 g, 4.56 mmoles, 1.0 equiv) and (S)-2-((S)-2-((tert-butoxycarbonyl)amino)-3-methylbutanamido)-5-ureidopentanoic acid (1.71 g, 4.56 mmoles, 1.0 equiv.) in 2:1 CH₂Cl₂/MeOH (60 mL) was added ethyl 2-ethoxyquinoline-1(2H)-carboxylate (2.256 g, 9.12 mmoles, 2.0 equiv.). The homogeneous solution was stirred for 16 hours at which time additional (S)-2-((S)-2-((tert-butoxycarbonyl)amino)-3-methylbutanamido)-5-ureidopentanoic acid (0.340 g, 0.2 equiv.) and ethyl 2-ethoxyquinoline-1(2H)-carboxylate (0.452 g, 0.4 equiv.) were added to drive the reaction to completion. After stirring for an additional 5 hours the volatiles were removed in vacuo and after purification by ISCO SiO₂ chromatography (0-5% MeOH/CH₂Cl₂) (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate was obtained (2.95 g, 65%). LCMS: MH+=984.1; Rt=1.54 min (2 min acidic method-Method A).

### Step 7: Synthesis of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate

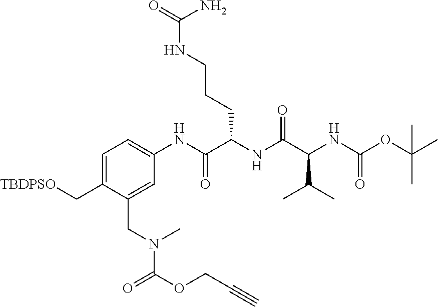

To (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (2.05 g, 2.085 mmol, 1.0 equiv) in THF (10 mL) was added 2.0 M dimethyl amine in MeOH (10.42 mL, 20.85 mmol, 10 equiv.). After stirring for 16 hours the volatiles were removed in vacuo. The residue was dissolved in CH₂Cl₂ (20 mL) and DIEA (0.533 mL, 4.17 mmol, 2 equiv.) and propargyl chloroformate (0.264 mL, 2.71 mmol, 1.3 equiv.) were added. After stirring at rt for 16 hours the reaction was diluted with CH₂Cl₂ (20 mL), was washed with NaHCO₃ (sat.), NaCl (sat.), dried over MgSO₄, filtered, concentrated and purified by ISCO SiO₂ chromatography (0-15% MeOH/CH₂Cl₂) to yield prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (1.04 grams, 59%). LCMS: MH+=843.8; Rt=1.35 min (2 min acidic method-Method A).

### Step 8: Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate (LI-3)

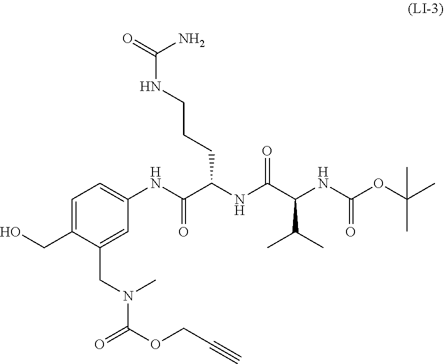 [SMILES: C#CCOC(=O)N(C)Cc1cc(NC(=O)[C@H](CCCNC(N)=O)NC(=O)[C@@H](NC(=O)OC(C)(C)C)C(C)C)ccc1CO]

To a 0° C. solution of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (1.6 g, 1.90 mmoles, 1.0 equiv.) in THF (10.0 mL) was added 1.0 M tetrabutylammonium fluoride in THF (3.80 mL, 3.80 mmoles, 2.0 equiv.). After warming to rt and stirring for 16 hours the volatiles were removed in vacuo, the residue was dissolved in EtOAc, was washed with NaHCO₃ (sat.), with NaCl (sat.), dried over MgSO₄, filtered, concentrated and the residue was purified by ISCO SiO₂ chromatography (0-30% MeOH/CH₂Cl₂) to yield prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate (LI-3) (1.0 g, 87%). LCMS: MH+=605.7; Rt=0.81 min (2 min acidic method-Method A).

### Example 1-4: Synthesis of tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate (LI-4)

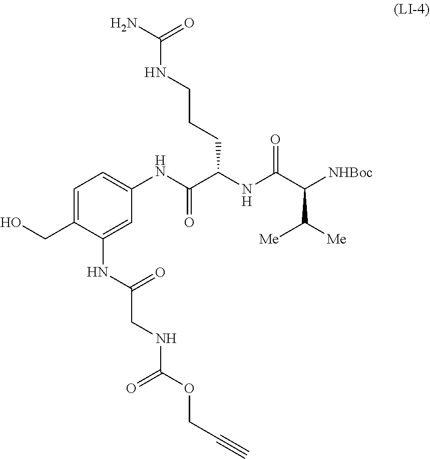 [SMILES: C#CCOC(=O)NCC(=O)Nc1cc(NC(=O)[C@H](CCCNC(N)=O)NC(=O)[C@@H](NC(=O)OC(C)(C)C)C(C)C)ccc1CO]

### Step 1: Synthesis of (9H-fluoren-9-yl)methyl (S)-(1-((4-(hydroxymethyl)-3-nitrophenyl)amino)-1-oxo-5-ureidopentan-2-yl)carbamate

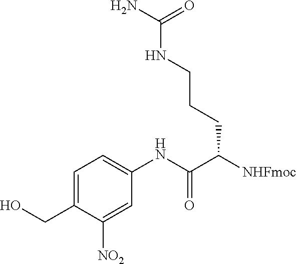

To a solution of (4-amino-2-nitrophenyl)methanol (10 g, 59.5 mmoles, 1.0 equiv.), (9H-fluoren-9-yl)methyl (S)-(1-amino-1-oxo-5-ureidopentan-2-yl)carbamate (23.64 g, 59.5 mmoles, 1.0 equiv.), and 1-hydroxy-7-azabenzotriazole (8.50 g, 62.4 mmoles, 1.05 equiv.) in DMF (50 mL) was added 1-(3-dimethylaminopropyl)-3-ethylcarbodiimide (11.97 g, 62.4 mmoles, 1.05 equiv.). After stirring at ambient temperature for 16 hours, the mixture was poured into water (4 L) and stirred for 30 minutes. The resulting solid was filtered, rinsed with water, and dried under vacuum. (9H-Fluoren-9-yl)methyl (S)-(1-((4-(hydroxymethyl)-3-nitrophenyl)amino)-1-oxo-5-ureidopentan-2-yl)carbamate was obtained (31.49 g, 57.5 mmoles, 97%). LCMS: MH+=548; Rt=2.02 min (5 min acidic method-Method C).

### Step 2: Synthesis of (S)-2-amino-N-(4-(hydroxymethyl)-3-nitrophenyl)-5-ureidopentanamide

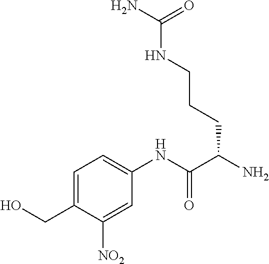 [SMILES: NC(=O)NCCC[C@H](N)C(=O)Nc1ccc(CO)c([N+](=O)[O-])c1]

To a solution of (9H-Fluoren-9-yl)methyl (S)-(1-((4-(hydroxymethyl)-3-nitrophenyl)amino)-1-oxo-5-ureidopentan-2-yl)carbamate (31.49 g, 57.5 mmoles, 1.0 equiv.) in DMF (50 mL) was added dimethylamine (2 M in MeOH, 331 mL, 661 mmoles, 11.5 equiv.). After stirring at ambient temperature for 24 hours, the volatiles were removed under vacuum and the resulting residue was triturated with diethyl ether (3×2 L). The resulting residue was dried under vacuum and (S)-2-amino-N-(4-(hydroxymethyl)-3-nitrophenyl)-5-ureidopentanamide was obtained (21.85 g, 57.5 mmol, 99%). LCMS: MH+=326.4; Rt=0.35 min (2 min acidic method-Method A).

### Step 3: Synthesis of tert-butyl ((S)-1-(((S)-1-((4-(hydroxymethyl)-3-nitrophenyl)amino)-1-oxo-5-ureidopentan-2-yl)amino)-3-methyl-1-oxobutan-2-yl)carbamate

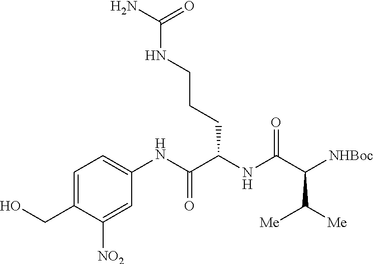 [SMILES: CC(C)[C@H](NC(=O)OC(C)(C)C)C(=O)N[C@@H](CCCNC(N)=O)C(=O)Nc1ccc(CO)c([N+](=O)[O-])c1]

To a solution of (S)-2-amino-N-(4-(hydroxymethyl-3-nitrophenyl-5-ureidopentanamide (10.89 g, 28.8 mmoles, 1.0 equiv.), (tert-butoxycarbonyl)-L-valine (6.25 g, 28.8 mmoles, 1.0 equiv.), and 1-hydroxy-7-azabenzotriazole (3.92 g, 28.8 mmoles, 1.0 equiv.) in DMF (40 mL) was added 1-(3-dimethylaminopropyl)-3-ethylcarbodiimide (5.52 g, 28.8 mmoles, 1.0 equiv.). After stirring at ambient temperature for 24 hours, the mixture was added dropwise to water (2 L), stirred for 30 minutes, and cooled to 4° C. overnight. The mixture was saturated with NaCl, and the resulting solids were filtered off and dried under vacuum. Tert-butyl ((S)-1-(((S)-1-((4-(hydroxymethyl)-3-nitrophenyl)amino)-1-oxo-5-ureidopentan-2-yl)amino)-3-methyl-1-oxobutan-2-yl)carbamate was obtained (11.96 g, 22.8 mmoles, 79%). LCMS: MH+=525.4; Rt=0.79 min (2 min acidic method-Method A).

### Step 4: Synthesis of tert-butyl ((S)-1-(((S)-1-((4-(((tert-butyldimethylsilyl)oxy)methyl)-3-nitrophenyl)amino)-1-oxo-5-ureidopentan-2-yl)amino)-3-methyl-1-oxobutan-2-yl)carbamate

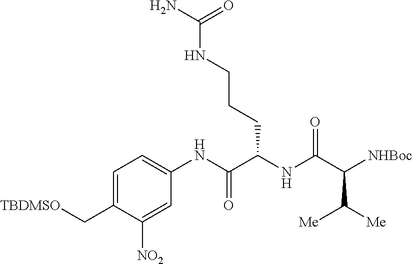 [SMILES: CC(C)[C@H](NC(=O)OC(C)(C)C)C(=O)N[C@@H](CCCNC(N)=O)C(=O)Nc1ccc(CO[Si](C)(C)C(C)(C)C)c([N+](=O)[O-])c1]

To a suspension of tert-butyl ((S)-1-(((S)-1-((4-(hydroxymethyl)-3-nitrophenyl)amino)-1-oxo-5-ureidopentan-2-yl)amino)-3-methyl-1-oxobutan-2-yl)carbamate (11.96 g, 22.8 mmoles, 1.0 equiv.) and imidazole (15.52 g, 228 mmol, 10 equiv.) in DMF (31 mL) was added tert-butyldimethylchlorosilane (13.68 g, 90.76 mmol, 4.0 equiv.). The resulting mixture was stirred at ambient temperature for 48 hours, then heated at 45° C. for 4 hours. The mixture was poured into water and stirred for 96 hours. Solids were filtered and washed with water (2×100 mL) and dried under vacuum. After purification by SiO₂ ISCO chromatography (0-30% methanol/dichloromethane), tert-butyl ((S)-1-(((S)-1-((4-(((tert-butyldimethylsilyl)oxy)methyl)-3-nitrophenyl)amino)-1-oxo-5-ureidopentan-2-yl)amino)-3-methyl-1-oxobutan-2-yl)carbamate was obtained (8.02 g, 12.56 mmoles, 55%). LCMS: MH+=639.6; Rt=1.22 min (2 min acidic method-Method A).

### Step 5: Synthesis of tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate

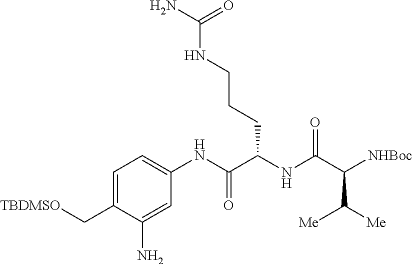 [SMILES: CC(C)[C@H](NC(=O)OC(C)(C)C)C(=O)N[C@@H](CCCNC(N)=O)C(=O)Nc1ccc(CO[Si](C)(C)C(C)(C)C)c(N)c1]

To a solution of tert-butyl ((S)-1-(((S)-1-((4-(((tert-butyldimethylsilyl)oxy)methyl)-3-nitrophenyl)amino)-1-oxo-5-ureidopentan-2-yl)amino)-3-methyl-1-oxobutan-2-yl)carbamate (8.02 g, 12.56 mmoles, 1.0 equiv.) in methanol (250 mL) under a nitrogen atmosphere was added palladium on carbon (10 wt %, 2.00 g, 1.884 mmoles, 0.15 equiv.). The mixture was placed under 1 atm dihydrogen and allowed to stir at ambient temperature for 18 hours. The mixture was filtered through celite and dried under vacuum. After purification by SiO₂ ISCO chromatography (0-40% methanol/dichloromethane), tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate was obtained (4.82 g, 7.92 mmol, 63%). LCMS: MH+=609.6; Rt=2.65 min (5 min acidic method-Method C).

### Step 6: Synthesis of tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate (LI-4)

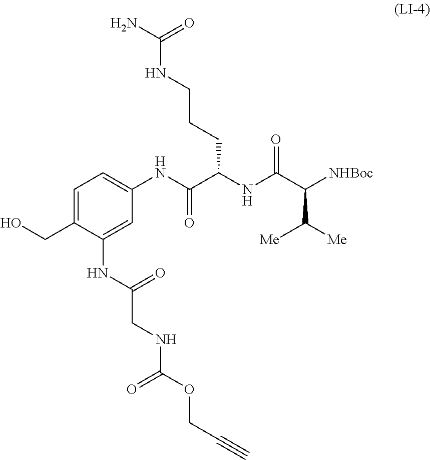 [SMILES: C#CCOC(=O)NCC(=O)Nc1cc(NC(=O)[C@H](CCCNC(N)=O)NC(=O)[C@@H](NC(=O)OC(C)(C)C)C(C)C)ccc1CO]

### Step 6a):

To a solution of glycine (3.19 g, 42.5 mmoles, 1.0 equiv.) in 2 M aqueous sodium hydroxide solution (63.3 mL, 127 mmoles NaOH, 3.0 equiv.) was added propargyl chloroformate (5.0 g, 42.5 mmoles, 1.0 equiv.). The resulting mixture was stirred at ambient temperature for 3 hours. The mixture was extracted with ethyl acetate (3×250 mL). The combined organic layers were dried over magnesium sulfate, filtered and the volatiles removed under vacuum. After drying, ((prop-2-yn-1-yloxy)carbonyl)glycine,

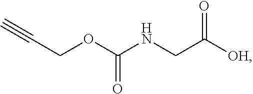

was obtained (3.97 g, 25.3 mmoles, 59%). ¹H NMR (400 MHz, DMSO-d₆) δ ppm 3.48 (t, J=2.40 Hz, 1H) 3.66 (d, J=6.19 Hz, 2H) 4.63 (d, J=2.40 Hz, 2H) 7.63 (t, J=6.13 Hz, 1H) 12.57 (br s, 1H).

### Step 6b):

To a solution of tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate (2.7 g, 4.43 mmoles, 1.0 equiv.) in DMF (5 mL) were added ((prop-2-yn-1-yloxy)carbonyl)glycine (0.732 g, 4.66 mmoles, 1.05 equiv.), 1-hydroxy-7-azabenzotriazole (0.664 g, 4.88 mmoles, 1.1 equiv.), and 1-(3-dimethylaminopropyl)-3-ethylcarbodiimide hydrochloride (0.935 g, 4.88 mmoles, 1.1 equiv). The resulting mixture was stirred at ambient temperature for 1 hour, then dripped into water (500 mL) and stirred for a further 20 minutes. The resulting precipitate was filtered, washed with water, and dried under vacuum. After purification by SiO₂ ISCO chromatography (0-50% methanol/dichloromethane), tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate (LI-4) was obtained (1.52 g, 2.40 mmoles, 54%). LCMS: MH+=634.6; Rt=1.97 min (5 min acidic method-Method C). ¹H NMR (400 MHz, DMSO-d₆) δ ppm 0.76-0.91 (m, 6H) 1.30-1.47 (m, 11H) 1.51-1.73 (m, 2H) 1.87-2.00 (m, 1H) 2.89-3.07 (m, 2H) 3.50 (t, J=2.32 Hz, 1H) 3.73-3.87 (m, 3H) 4.37-4.47 (m, 3H) 4.65 (d, J=2.45 Hz, 2H) 5.30 (t, J=5.44 Hz, 1H) 5.38 (s, 2H) 5.96 (t, J=5.81 Hz, 1H) 6.72 (br d, J=8.93 Hz, 1H) 7.25 (d, J=8.44 Hz, 1H) 7.45 (dd, J=8.25, 2.02 Hz, 1H) 7.78 (br t, J=5.87 Hz, 1H) 7.87-8.00 (m, 2H) 9.51 (s, 1H) 10.04 (s, 1H).

### Example 1-5: Synthesis of tert-butyl ((S)-1-(((S)-1-((3-(di(prop-2-yn-1-yl)carbamoyl)-4-(hydroxymethyl)phenyl)amino)-1-oxo-5-ureidopentan-2-yl)amino)-3-methyl-1-oxobutan-2-yl)carbamate (LI-5)

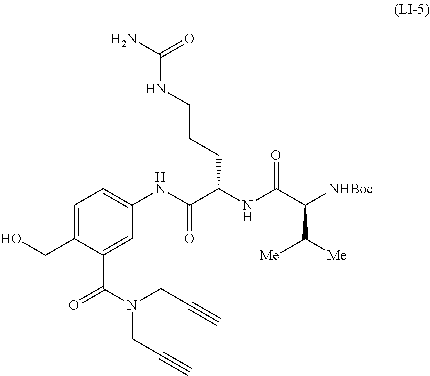 [SMILES: C#CCN(CC#C)C(=O)c1cc(NC(=O)[C@H](CCCNC(N)=O)NC(=O)[C@@H](NC(=O)OC(C)(C)C)C(C)C)ccc1CO]

### Step 1: Synthesis of 2-(((tert-butyldiphenylsilyl)oxy)methyl)-5-nitro-N,N-di(prop-2-yn-1-yl)benzamide

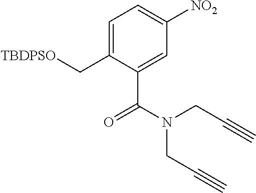

To a solution of 2-(((tert-butyldiphenylsilyl)oxy)methyl)-5-nitrobenzoic acid (1.00 g, 2.30 mmoles, 1.0 equiv.) and dipropargylamine (0.257 g, 2.76 mmoles, 1.2 equiv.) in dichloromethane (6 mL) were added (1-[bis(dimethylamino)methylene]-1H-1,2,3-triazolo[4,5-b]pyridinium 3-oxid hexafluorophosphate (1.048 g, 2.76 mmoles, 1.2 equiv.) and N,N-diisopropylethylamine (0.445 g, 3.44 mmoles, 1.5 equiv.). The resulting mixture was stirred at ambient temperature for 1 hour, then diluted with water, extracted with diethyl ether (3×25 mL), dried over sodium sulfate and concentrated. After purification by SiO₂ ISCO chromatography (0-100% ethyl acetate/heptanes), 2-(((tert-butyldiphenylsilyl)oxy)methyl)-5-nitro-N,N-di(prop-2-yn-1-yl)benzamide was obtained (1.08 g, 2.115 mmoles, 92%). 1H NMR (400 MHz, Chloroform-d) b 8.35 (dd, J=8.6, 2.3 Hz, 1H), 8.20 (d, J=2.3 Hz, 1H), 8.02-7.92 (m, 1H), 7.71-7.62 (m, 4H), 7.51-7.35 (m, 6H), 4.87 (s, 2H), 4.39 (s, 2H), 3.80 (s, 2H), 2.21 (s, 1H), 2.08 (d, J=7.7 Hz, 1H), 1.13 (s, 9H).

### Step 2: Synthesis of 5-amino-2-(((tert-butyldiphenylsilyl)oxy)methyl)-N,N-di(prop-2-yn-1-yl)benzamide

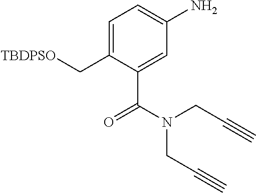

To a stirred suspension of 2-(((tert-butyldiphenylsilyl)oxy)methyl)-5-nitro-N,N-di(prop-2-yn-1-yl)benzamide (1.08 g, 2.115 mmoles, 1.0 equiv.) in ethanol (4 mL) and water (4 mL) was added zinc powder (0.553 g, 8.46 mmoles, 4 equiv.) and ammonium chloride (0.453 g, 8.46 mmoles, 4 equiv.). The resulting mixture was stirred at ambient temperature for 24 hours, then diluted with water and extracted with ethyl acetate (3×25 mL). The combined organic layers were dried over sodium sulfate, filtered and concentrated. After drying under vacuum, 5-amino-2-(((tert-butyldiphenylsilyl)oxy)methyl)-N,N-di(prop-2-yn-1-yl)benzamide was obtained (972 mg, 2.02 mmoles, 96%). LCMS: MH+=481.4; Rt=1.33 min (2 min acidic method-Method A).

### Step 3: Synthesis of (S)-5-(2-amino-5-ureidopentanamido)-2-(((tert-butyldiphenylsilyl)oxy)methyl)-N,N-di(prop-2-yn-1-yl)benzamide

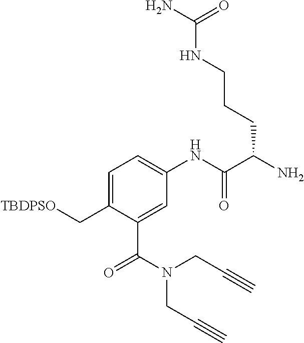

To a solution of 5-amino-2-(((tert-butyldiphenylsilyl)oxy)methyl)-N,N-di(prop-2-yn-1-yl)benzamide (972 mg, 2.02 mmoles, 1.0 equiv.), (9H-fluoren-9-yl)methyl (S)-(1-amino-1-oxo-5-ureidopentan-2-yl)carbamate (804 mg, 2.02 mmoles, 1.0 equiv.), and (1-[bis(dimethylamino)methylene]-1H-1,2,3-triazolo[4,5-b]pyridinium 3-oxid hexafluorophosphate (846 mg, 2.22 mmoles, 1.1 equiv.) in DMF (4 mL) was added N,N-diisopropylethylamine (0.53 mL, 3.03 mmoles, 1.5 equiv.). The resulting mixture was stirred at ambient temperature for 18 hours, then poured into water (400 mL) and stirred for 3 hours. The precipitate was filtered and dried under vacuum, then dissolved in a 2 M solution of dimethylamine in tetrahydrofuran (2.02 mL, 4.04 mmoles, 2 equiv.) and stirred at ambient temperature for 4 hours. The volatiles were removed under vacuum and after purification by SiO₂ ISCO chromatography, (S)-5-(2-amino-5-ureidopentanamido)-2-(((tert-butyldiphenylsilyl)oxy)methyl)-N,N-di(prop-2-yn-1-yl)benzamide was obtained (1.018 g, 1.596 mmoles, 79%). LCMS: MH+=638.6; Rt=1.22 min (2 min acidic method-Method A).

### Step 4: Synthesis of tert-butyl ((S)-1-(((S)-1-((4-(((tert-butyldiphenylsilyl)oxy)methyl)-3-(di(prop-2-yn-1-yl)carbamoyl)phenyl)amino)-1-oxo-5-ureidopentan-2-yl)amino)-3-methyl-1-oxobutan-2-yl)carbamate

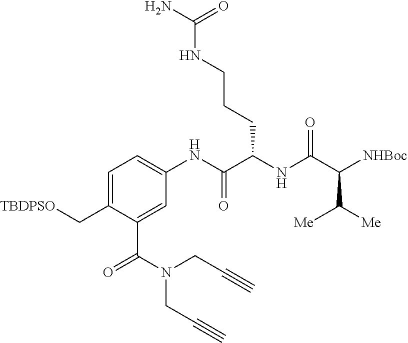

To a solution of (S)-5-(2-amino-b-ureidopentanamido)-2-(((tert-butyldiphenylsilyl)oxy)methyl)-N,N-di(prop-2-yn-1-yl)benzamide (1.00 g, 1.568 mmoles, 1.0 equiv.), (tert-butoxycarbonyl)-L-valine (0.341 g, 1.568 mmol, 1.0 equiv.), and (1-[bis(dimethylamino)methylene]-1H-1,2,3-triazolo[4,5-b]pyridinium 3-oxid hexafluorophosphate (0.656 g, 1.725 mmoles, 1.1 equiv.) in DMF (3 mL) was added N,N-diisopropylethylamine (0.41 mL, 2.352 mmoles, 1.5 equiv). After stirring at ambient temperature for 1 hour, the mixture was diluted with water (30 mL) and brine (30 mL) and extracted with ethyl acetate (3×50 mL). The combined organic layers were dried over sodium sulfate, filtered and concentrated. After purification of the resulting residue by SiO₂ ISCO chromatography (0-50% methanol/dichloromethane), tert-butyl ((S)-1-(((S)-1-((4-(((tert-butyldiphenylsilyl)oxy)methyl)-3-(di(prop-2-yn-1-yl)carbamoyl)phenyl)amino)-1-oxo-5-ureidopentan-2-yl)amino)-3-methyl-1-oxobutan-2-yl)carbamate was obtained (1.30 g, 1.553 mmol, 99%). LCMS: MH+=837.5; Rt=1.32 min (2 min acidic method-Method A).

### Step 5: Synthesis of tert-butyl ((S)-1-(((S)-1-((3-(di(prop-2-yn-1-yl)carbamoyl)-4-(hydroxymethyl)phenyl)amino)-1-oxo-5-ureidopentan-2-yl)amino)-3-methyl-1-oxobutan-2-yl)carbamate (LI-5)

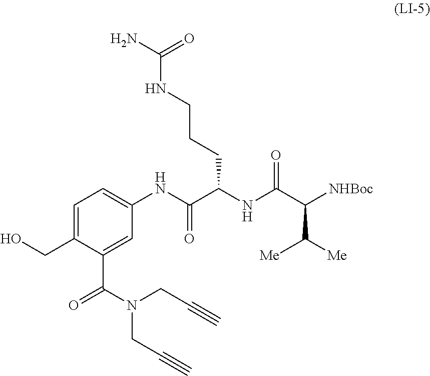 [SMILES: C#CCN(CC#C)C(=O)c1cc(NC(=O)[C@H](CCCNC(N)=O)NC(=O)[C@@H](NC(=O)OC(C)(C)C)C(C)C)ccc1CO]

To a stirred solution of tert-butyl ((S)-1-(((S)-1-((4-(((tert-butyldiphenylsilyl)oxy)methyl)-3-(di(prop-2-yn-1-yl)carbamoyl)phenyl)amino)-1-oxo-5-ureidopentan-2-yl)amino)-3-methyl-1-oxobutan-2-yl)carbamate (1.30 g, 1.553 mmol, 1.0 equiv.) in tetrahydrofuran (5 mL), a 1 M solution of tetrabutylammonium fluoride in tetrahydrofuran (3.11 mL, 3.11 mmoles, 2.0 equiv.) was added dropwise. After stirring at ambient temperature for 18 hours, the solvent was removed under vacuum. After purification by SiO₂ ISCO chromatography (0-50% methanol/dichloromethane), tert-butyl ((S)-1-(((S)-1-((3-(di(prop-2-yn-1-yl)carbamoyl)-4-(hydroxymethyl)phenyl)amino)-1-oxo-5-ureidopentan-2-yl)amino)-3-methyl-1-oxobutan-2-yl)carbamate (LI-5) was obtained (0.703 g, 1.174 mmol, 76%). LCMS: MH+=599.4; Rt=0.76 min (2 min acidic method-Method A). 1H NMR (400 MHz, Methanol-d4) δ 7.71-7.59 (m, 2H), 7.52-7.43 (m, 1H), 4.51 (d, J=29.4 Hz, 4H), 4.11-4.04 (m, 2H), 3.95-3.85 (m, 1H), 3.28-3.06 (m, 2H), 2.76 (m, 2H), 2.11-2.03 (m, 1H), 1.97-1.83 (m, 1H), 1.75 (dtd, J=14.2, 9.4, 5.1 Hz, 1H), 1.70-1.51 (m, 3H), 1.44 (m, 9H), 1.00-0.90 (m, 6H).

### Example 2: Synthesis of Drug Component

### Example 2-1: Synthesis of (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl) pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (P1)

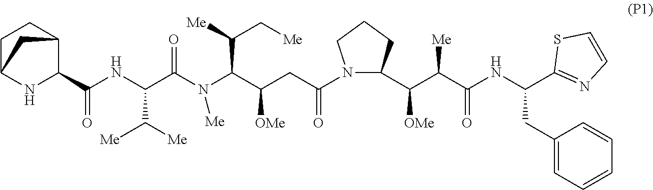 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H]1N[C@@H]2CC[C@H]1C2)C(C)C]

### Step 1: Synthesis of methyl (2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanoate

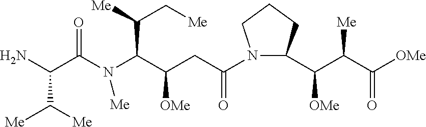 [SMILES: CC[C@H](C)[C@@H]([C@@H](CC(=O)N1CCC[C@H]1[C@H](OC)[C@@H](C)C(=O)OC)OC)N(C)C(=O)[C@@H](N)C(C)C]

To a stirring solution of methyl (2R,3R)-3-((S)-1-((3R,4S,5S)-4-((S)-2-(((benzyloxy)carbonyl)amino)-N,3-dimethylbutanamido)-3-methoxy-5-methylheptanoyl)pyrrolidin-2-yl)-3-methoxy-2-methylpropanoate (2.00 g, 3.23 mmoles, 1.0 equiv.) in methanol (50 mL) was added palladium on carbon (10 wt %, 0.343 g, 0.323 mmoles, 0.1 equiv.). Dry nitrogen was bubbled through the reaction for 5 minutes, then the reaction was put under 1 atm H2. After stirring at ambient temperature for 1 hour, dry nitrogen was bubbled through the mixture for 5 minutes. The mixture was filtered through celite, rinsing with 50 mL methanol. The filtrate was concentrated and dried under vacuum, and methyl (2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanoate was obtained (1.55 g, 3.19 mmoles, 99%). LCMS: MH+=486.1; Rt=0.93 min (2 min acidic method-Method A).

### Step 2: Synthesis of tert-butyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate

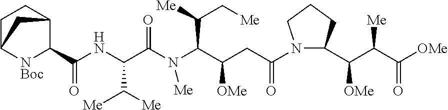 [SMILES: CC[C@H](C)[C@@H]([C@@H](CC(=O)N1CCC[C@H]1[C@H](OC)[C@@H](C)C(=O)OC)OC)N(C)C(=O)[C@@H](NC(=O)[C@@H]1[C@H]2CC[C@H](C2)N1C(=O)OC(C)(C)C)C(C)C]

To a stirred solution of methyl (2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanoate (1.55 g, 3.19 mmoles, 1.00 equiv.), (1R,3S,4S)-2-(tert-butoxycarbonyl)-2-azabicyclo[2.2.1]heptane-3-carboxylic acid (0.77 g, 3.19 mmoles, 1.00 equiv.), and 1-hydroxy-7-azabenzotriazole (0.500 g, 3.67 mmoles, 1.15 equiv.) in DMF (6 mL) was added 1-(3-dimethylaminopropyl)-3-ethylcarbodiimide hydrochloride (0.704 g, 3.67 mmoles, 1.15 equiv.). The resulting mixture was stirred at ambient temperature for 18 hours, then diluted with water (100 mL) and extracted with ethyl acetate (3×50 mL). The combined organic layers were washed with 1 M aqueous sodium hydroxide (50 mL) and brine (50 mL), then dried over sodium sulfate, filtered, and concentrated. After drying under vacuum, tert-butyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate was obtained (2.20 g, 3.10 mmoles, 97%). LCMS: MH+=709.5; Rt=1.23 min (2 min basic method-Method B).

### Step 3: Synthesis of (2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid

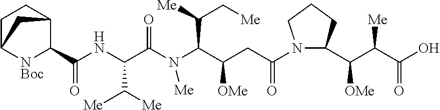 [SMILES: CC[C@H](C)[C@@H]([C@@H](CC(=O)N1CCC[C@H]1[C@H](OC)[C@@H](C)C(=O)O)OC)N(C)C(=O)[C@@H](NC(=O)[C@@H]1[C@H]2CC[C@H](C2)N1C(=O)OC(C)(C)C)C(C)C]

A solution of lithium hydroxide monohydrate (0.26 g, 6.21 mmoles, 2.0 equiv.) in water (5 mL) was added dropwise to a solution of tert-butyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (2.20 g, 3.10 mmoles, 1.0 equiv.) in tetrahydrofuran (5 mL) and methanol (5 mL). After addition was complete, the mixture was stirred at ambient temperature for 18 hours. The mixture was then quenched with 1 M aqueous HCl (6.5 mL) and volatiles were removed under vacuum. The resulting residue was partitioned between ethyl acetate (50 mL) and brine (100 mL). The layers were separated and the aqueous layer was extracted with ethyl acetate (2×50 mL). The combined organic layers were dried over sodium sulfate, filtered, and concentrated. After drying under vacuum, (2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid was obtained (1.96 g, 2.82 mmoles, 91%). LCMS: MH+=695.5; Rt=0.73 min (2 min basic method-Method B).

### Step 4: Synthesis of tert-butyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

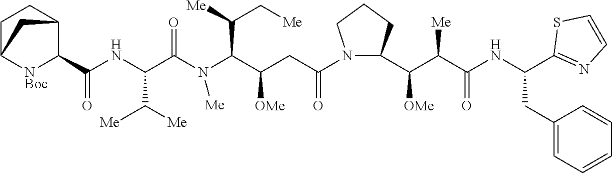 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OC(C)(C)C)C(C)C]

To a solution of (2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid (250 mg, 0.360 mmoles, 1.0 equiv.), (S)-2-phenyl-1-(thiazol-2-yl)ethan-1-amine hydrochloride (95 mg, 0.396 mmoles, 1.1 equiv.), and (1-[bis(dimethylamino)methylene]-1H-1,2,3-triazolo[4,5-b]pyridinium 3-oxid hexafluorophosphate (150 mg, 0.396 mmoles, 1.1 equiv.) in DMF (1 mL) was added N,N-diisopropylethylamine (0.25 mL, 1.44 mmoles, 4 equiv.). The resulting mixture was stirred at ambient temperature for 1 hour. The mixture was poured into brine (50 mL) and extracted with ethyl acetate (3×25 mL). The combined organic layers were dried over sodium sulfate, filtered, and concentrated. After purification by SiO₂ ISCO chromatography (0-20% methanol/dichloromethane), tert-butyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained (317 mg, 0.360 mmoles, 99%). LCMS: MH+=881.5; Rt=1.23 min (2 min basic method-Method B).

### Step 5: Synthesis of (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (P1)

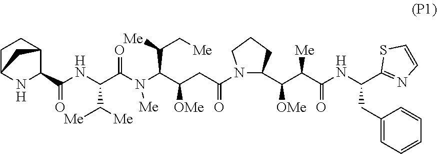 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H]1N[C@@H]2CC[C@H]1C2)C(C)C]

To a solution of tert-butyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (317 mg, 0.360 mmoles, 1.0 equiv.) in dichloromethane (5 mL) was added trifluoroacetic acid (1 mL). The resulting mixture was stirred at ambient temperature for 1.5 hours, then volatiles were removed under vacuum. The residue was partitioned between ethyl acetate (25 mL) and 1 M aqueous NaOH saturated with NaCl (50 mL). The layers were separated and the aqueous layer was extracted with ethyl acetate (2×25 mL). The combined organic layers were dried over sodium sulfate, filtered and concentrated. After purification by RP-HPLC ISCO gold chromatography (10-100% MeCN/H2O, 0.1% TFA modifier), (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (P1) was obtained (268 mg, 0.299 mmoles, 83%). LCMS: MH+=781.5; Rt=1.11 min (2 min basic method-Method B).

### Example 3: Synthesis of Exemplary Linker-Drug Compounds

### Example 3-1: Synthesis of 2-(((1-(2,5,8,11,14,17,20,23-octaoxapentacosan-25-yl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L4-P1)

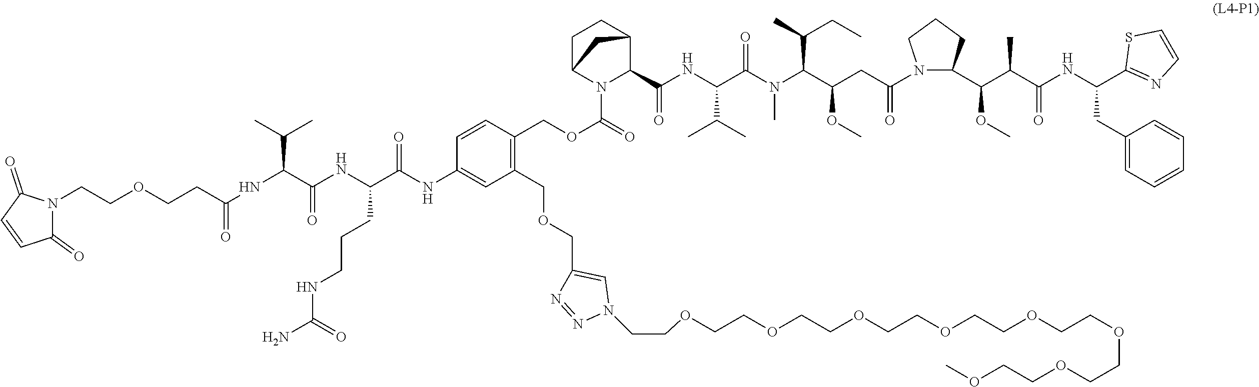 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOC)nn1)C(C)C]

### Step 1: Synthesis of tert-butyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate

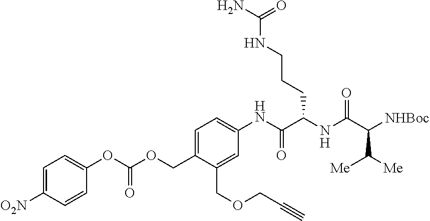 [SMILES: C#CCOCc1cc(NC(=O)[C@H](CCCNC(N)=O)NC(=O)[C@@H](NC(=O)OC(C)(C)C)C(C)C)ccc1COC(=O)Oc1ccc([N+](=O)[O-])cc1]

To a stirred solution of tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (LI-1) (500 mg, 0.913 mmoles, 1.0 equiv.) in DMF (2 mL) were added bis(4-nitrophenyl)carbonate (306 mg, 1.004 mmoles, 1.1 equiv.) and N,N-diisopropylethylamine (0.32 mL, 1.826 mmol, 2.0 equiv.). The resulting solution was stirred at ambient temperature for 1 hour. The reaction mixture was diluted with 4 mL DMSO, and after purification by RP-HPLC ISCO gold chromatography (10-100% acetonitrile/water, 0.1% trifluoroacetic acid modifier), tert-butyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate was obtained (550 mg, 0.772 mmoles, 85%). LCMS: MNa+=735.4; Rt=1.05 min (2 min acidic method-Method A).

### Step 2: Synthesis of 4-((S)-2-((S)-2-((tert-butoxycarbonyl)amino)-3-methylbutanamido)-5-ureidopentanamido)-2-((prop-2-yn-1-yloxy)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

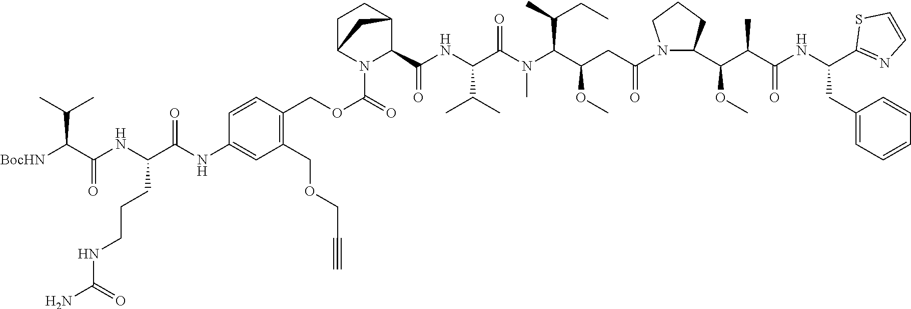 [SMILES: C#CCOCc1cc(NC(=O)[C@H](CCCNC(N)=O)NC(=O)[C@@H](NC(=O)OC(C)(C)C)C(C)C)ccc1COC(=O)N1[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C]

To a solution of (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (P1) (50 mg, 0.064 mmoles, 1.0 equiv.) in DMF (1 mL) were added tert-butyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate (45.6 mg, 0.064 mmoles, 1.0 equiv.) and N,N-diisopropylethylamine (0.112 mL, 0.640 mmoles, 10 equiv.). The resulting solution was stirred at ambient temperature for 18 hours, then diluted with 2 mL DMSO. After purification by RP-HPLC ISCO gold chromatography (10-100% acetonitrile/water, 0.1% trifluoroacetic acid modifier), 4-((S)-2-((S)-2-((tert-butoxycarbonyl)amino)-3-methylbutanamido)-5-ureidopentanamido)-2-((prop-2-yn-1-yloxy)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained (56 mg, 0.041 mmoles, 64%). LCMS: MH+=1353.3; Rt=1.13 min (2 min acidic method-Method A).

### Step 3: Synthesis of 2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylat

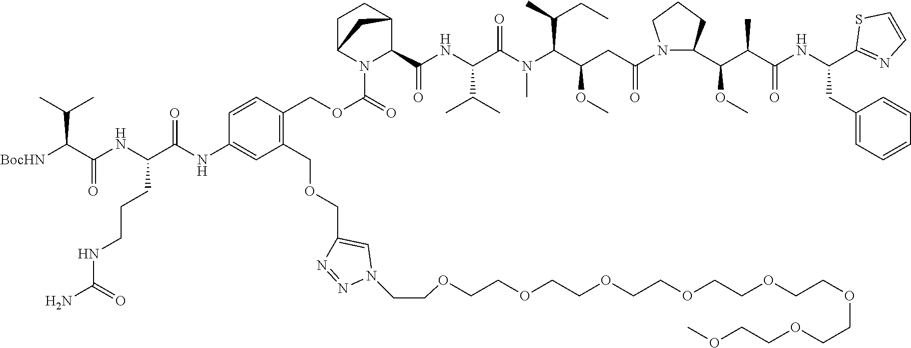 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)OC(C)(C)C)C(C)C)cc1COCc1cn(CCOCCOCCOCCOCCOCCOCCOCCOC)nn1)C(C)C]

To 4-((S)-2-((S)-2-((tert-butoxycarbonyl)amino)-3-methylbutanamido)-5-ureidopentanamido)-2-((prop-2-yn-1-yloxy)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (55 mg, 0.041 mmoles, 1.0 equiv) and 25-azido-2,5,8,11,14,17,20,23-octaoxapentacosane (33 mg, 0.082 mmoles, 2.0 equiv.) was added t-BuOH (1 mL). The mixture was degassed via house vacuum and purged to a balloon of N₂ via a 3 way stopcock. Degas/purge was repeated 3 times. A 16 mg/mL aqueous solution of sodium abscorbate (0.75 mL, 0.061 mmoles, 1.5 equiv.) was added and the solution was degassed and purged to N₂ three times. A 4 mg/mL aqueous solution of copper sulfate (0.75 mL, 0.0123 mmoles, 0.3 equiv.) was added and the solution was degassed and purged to N₂ three times. After stirring under N₂ for 3 hours the reaction was diluted with DMSO (3 mL) and was purified by RP-HPLC ISCO gold chromatography (10-100% MeCN/H2O, 0.1% TFA modifier). Upon lyophilization 2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino) propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (63 mg, 0.036 mmoles, 88%) was obtained. LCMS: [M+2H]2+=883.1; Rt=1.11 min (2 min acidic method-Method A).

### Step 4: Synthesis of 2-(((1-(2,5,8,11,14,17,20,23-octaoxapentacosan-25-yl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L4-P1)

To 2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (63 mg, 0.036 mmoles, 1.0 equiv.) was added 25% TFA/CH₂Cl₂ (2 mL). After standing for 45 minutes the volatiles were removed in vacuo, CH₂Cl₂ was added the volatiles were removed in vacuo and the residue was dried under vacuum. The residue was dissolved in DMF (1 mL) and N,N-diisopropylethylamine (93 μL, 0.540 mmoles, 15 equiv.) and 2,5-dioxopyrrolidin-1-yl 3-(2-(2,5-dioxo-2,5-dihydro-1H-pyrrol-1-yl)ethoxy)propanoate (22 mg, 0.072 mmoles, 2 equiv. ) was added. After stirring for 2 hours the solution was diluted with DMSO (2 mL) and was purified by RP-ISCO gold chromatography. Upon lyophilization 2-(((1-(2,5,8,11,14,17,20,23-octaoxapentacosan-25-yl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L4-P1) (6.0 mg, 3.16 μmol, 9%) was obtained. HRMS: M+=1858.9881, Rt=2.49 min (5 min acidic method-Method D).

### Example 3-2: Synthesis of 2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L2-P1)

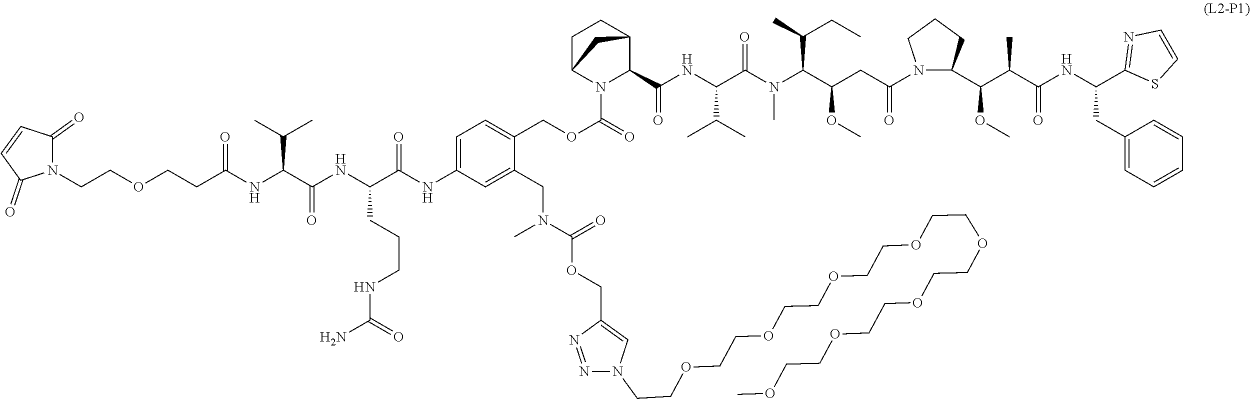 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOC)nn1)C(C)C]

### Step 1: Synthesis of prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate

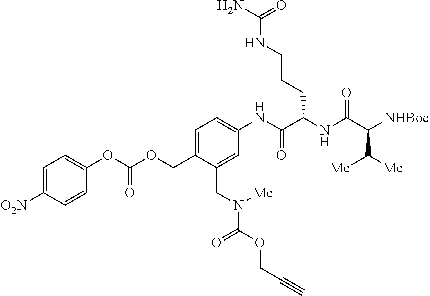 [SMILES: C#CCOC(=O)N(C)Cc1cc(NC(=O)[C@H](CCCNC(N)=O)NC(=O)[C@@H](NC(=O)OC(C)(C)C)C(C)C)ccc1COC(=O)Oc1ccc([N+](=O)[O-])cc1]

Prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate was obtained using the procedure described in Example 3-1, step 1, however tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (LI-1) was replaced with prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate (LI-3) (300 mg, 0.496 mmoles, 1.0 equiv.) and N,N-diisopropylethylamine was omitted. Prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate: (347 mg, 0.451 mmoles, 91%). LCMS: MH+=770.3, Rt=2.39 min (5 min acidic method-Method C). 1H NMR (400 MHz, DMSO-d6) δ 10.19 (s, 1H), 8.35-8.28 (m, 2H), 8.00 (d, J=7.6 Hz, 1H), 7.72-7.64 (m, 1H), 7.61-7.54 (m, 2H), 7.41 (d, J=8.4 Hz, 2H), 6.73 (d, J=9.0 Hz, 1H), 5.95 (t, J=5.9 Hz, 1H), 5.38 (s, 2H), 5.30 (s, 2H), 4.71 (s, 2H), 4.59 (s, 2H), 4.42 (q, J=7.3 Hz, 1H), 3.87-3.79 (m, 1H), 3.51 (d, J=22.1 Hz, 1H), 3.07-2.89 (m, 2H), 2.85 (s, 3H), 2.00-1.88 (m, 1H), 1.75-1.43 (m, 3H), 1.42-1.32 (m, 10H), 0.83 (dd, J=16.0, 6.7 Hz, 6H).

### Step 2: Synthesis of 4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

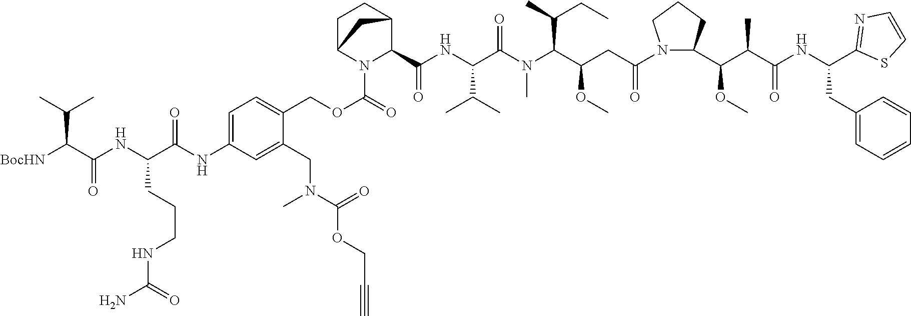 [SMILES: C#CCOC(=O)N(C)Cc1cc(NC(=O)[C@H](CCCNC(N)=O)NC(=O)[C@@H](NC(=O)OC(C)(C)C)C(C)C)ccc1COC(=O)N1[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C]

4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained using the procedure described in Example 3-1, step 2, however tert-butyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate was replaced with prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (43 mg, 0.056 mmoles, 1.0 equiv.). 4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: (33.7 mg, 0.024 mmoles, 43%). LCMS: [M+2H]2+707.0, Rt=2.55 min (5 min acidic method-Method C).

### Step 3: Synthesis of 2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

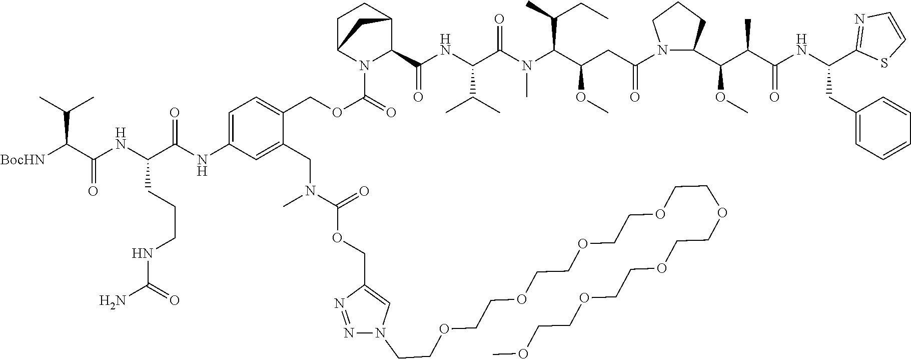 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)OC(C)(C)C)C(C)C)cc1CN(C)C(=O)OCc1cn(CCOCCOCCOCCOCCOCCOCCOCCOC)nn1)C(C)C]

2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained using the procedure described in Example 3-1, step 3, however 4-((S)-2-((S)-2-((tert-butoxycarbonyl)amino)-3-methylbutanamido)-5-ureidopentanamido)-2-((prop-2-yn-1-yloxy)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was replaced with 4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (33.7 mg, 0.024 mmoles, 1.0 equiv.), 2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: (25.1 mg, 0.014 mmoles, 57%). LCMS: [M+2H]2+911.1, Rt=2.47 min (5 min acidic method-Method C).

### Step 4: Synthesis of 2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L2-P1)

2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L2-P1) was obtained using the procedure described in Example 3-1, step 4, however 2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was replaced with 2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (19.9 mg, 0.011 mmoles, 1.0 equiv.) 2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L2-P1): (14.5 mg, 7.49 μmoles, 68%). HRMS: M+=1916.0000, Rt=2.50 min (5 min acidic method-Method D).

### Example 3-3: Synthesis of 2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L3-P1)

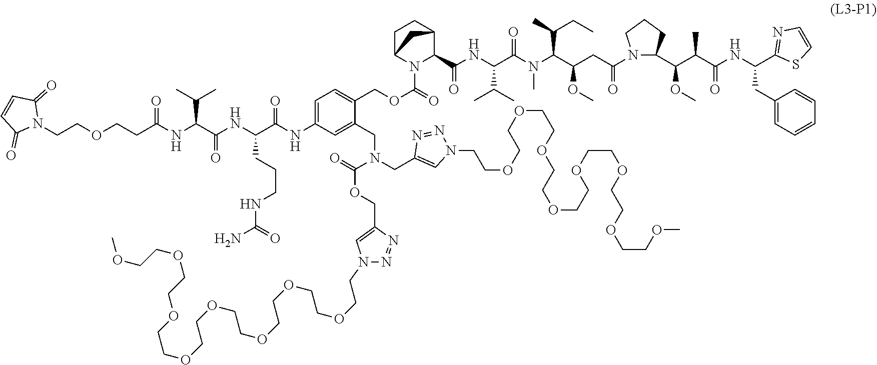 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1)C(C)C]

### Step 1: Synthesis of prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(prop-2-yn-1-yl)carbamate

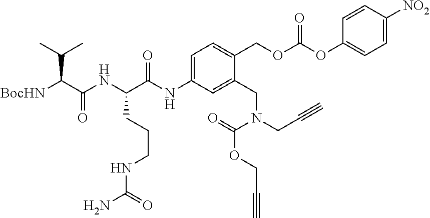 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1COC(=O)Oc1ccc([N+](=O)[O-])cc1]

Prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(prop-2-yn-1-yl)carbamate was obtained using the procedure described in Example 3-1, step 1, however tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (LI-1) was replaced with prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate (LI-2) (380 mg, 0.604 mmoles, 1.0 equiv.) and N,N-diisopropylethylamine was omitted. Prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(prop-2-yn-1-yl)carbamate: (374 mg, 0.472 mmoles, 78%). LCMS: MH+794.8, Rt=2.46 min (5 min acidic method-Method C).

### Step 2: Synthesis of 4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

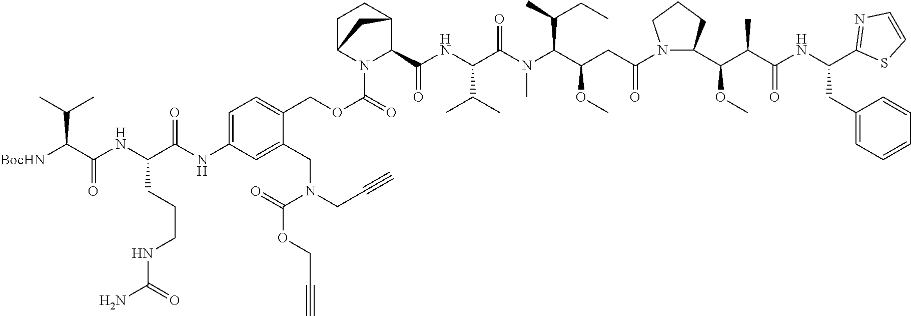 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1COC(=O)N1[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C]

4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained using the procedure described in Example 3-1, step 2, however tert-butyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate was replaced with prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(prop-2-yn-1-yl)carbamate (44.3 mg, 0.056 mmoles, 1.0 equiv.). 4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: (71.2 mg, 0.050 mmoles, 89%). HRMS: MH+=1435.7600 Rt=2.70 min (5 min acidic method-Method D).

### Step 3: Synthesis of 4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

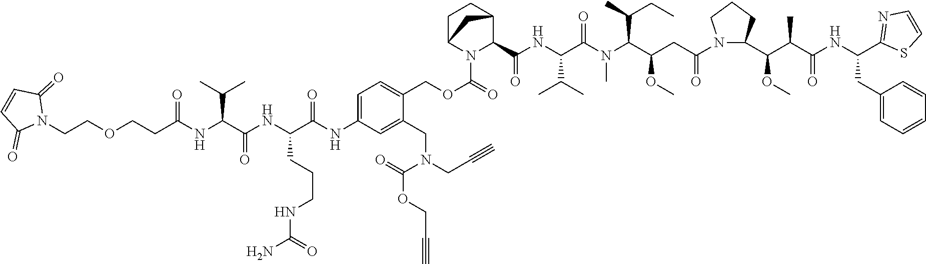 [SMILES: C#CCOC(=O)N(CC#C)Cc1cc(NC(=O)[C@H](CCCNC(N)=O)NC(=O)[C@@H](NC(=O)CCOCCN2C(=O)C=CC2=O)C(C)C)ccc1COC(=O)N1[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C]

4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: (53.9 mg, 0.032 mmoles, 64%). HRMS: MH+=1530.7600 Rt=2.63 min (5 min acidic method-Method D).

### Step 4: Synthesis of 2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L3-P1)

2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L3-P1) was obtained using the procedure described in Example 3-1, step 4, however 2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was replaced with 4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yl((prop-2-yn-1-yloxy)carbonyl)amino)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (26.7 mg, 0.017 mmoles, 1.0 equiv.). 2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L3-P1): (21.1 mg, 8.26 μmoles, 47%). HRMS: MH+=2349.2400 Rt=2.51 min (5 min acidic method-Method D).

### Example 3-4: Synthesis of 2-(2-((((1-(2,5,8,11,14,17,20,23-octaoxapentacosan-25-yl)-1H-1,2,3-triazol-4-yl)methoxy)carbonyl)amino)acetamido)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L5-P1)

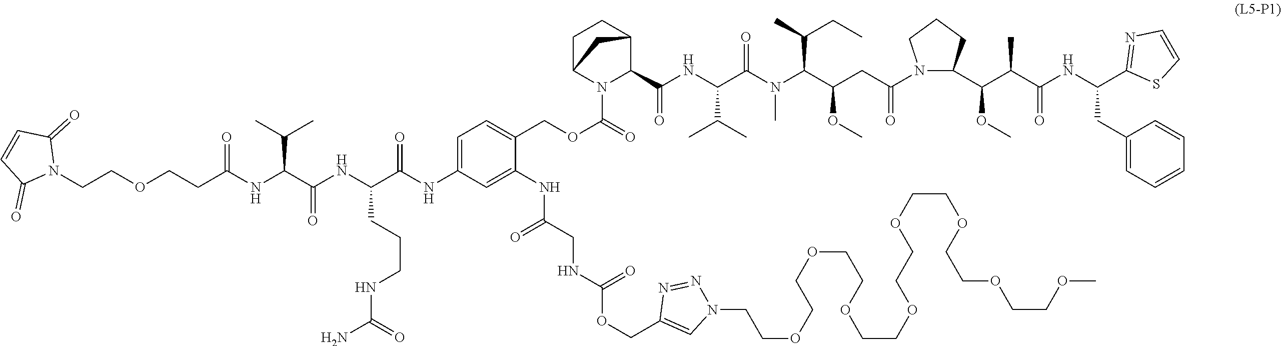 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCOC)nn1)C(C)C]

### Step 1: Synthesis of tert-butyl ((S)-3-methyl-1-(((S)-1-((4-((((4-nitrophenoxy)carbonyl)oxy)methyl)-3-(2-(((prop-2-yn-1-yloxy)carbonyl)amino)acetamido)phenyl)amino)-1-oxo-5-ureidopentan-2-yl)amino)-1-oxobutan-2-yl)carbamate

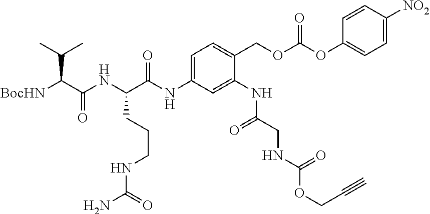 [SMILES: C#CCOC(=O)NCC(=O)Nc1cc(NC(=O)[C@H](CCCNC(N)=O)NC(=O)[C@@H](NC(=O)OC(C)(C)C)C(C)C)ccc1COC(=O)Oc1ccc([N+](=O)[O-])cc1]

Tert-butyl ((S)-3-methyl-1-(((S)-1-((4-((((4-nitrophenoxy)carbonyl)oxy)methyl)-3-(2-(((prop-2-yn-1-yloxy)carbonyl)amino)acetamido)phenyl)amino)-1-oxo-5-ureidopentan-2-yl)amino)-1-oxobutan-2-yl)carbamate was obtained using the procedure described in Example 3-1, step 1, however tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (LI-1) was replaced with tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate (LI-4) (552 mg, 0.871 mmoles, 1.0 equiv.), and N,N-diisopropylethylamine was omitted.

Tert-butyl ((S)-3-methyl-1-(((S)-1-((4-((((4-nitrophenoxy)carbonyl)oxy)methyl)-3-(2-(((prop-2-yn-1-yloxy)carbonyl)amino)acetamido)phenyl)amino)-1-oxo-5-ureidopentan-2-yl)amino)-1-oxobutan-2-yl)carbamate: (388 mg, 0.486 mmoles, 55%). LCMS: MH+799.7, Rt=2.14 min (5 min acidic method-Method C).

### Step 2: Synthesis of 4-((S)-2-((S)-2-((tert-butoxycarbonyl)amino)-3-methylbutanamido)-5-ureidopentanamido)-2-(2-(((prop-2-yn-1-yloxy)carbonyl)amino)acetamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

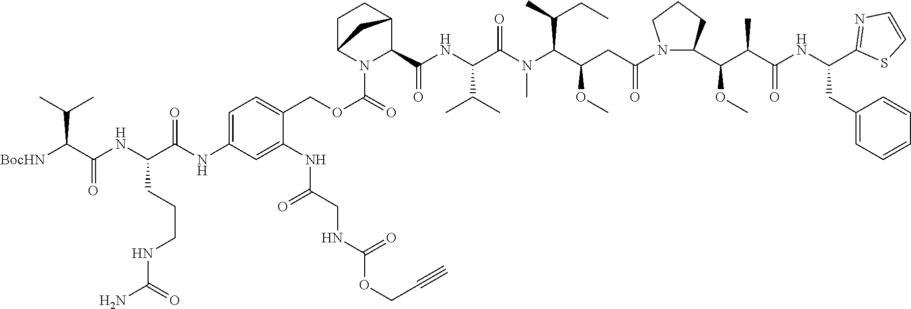 [SMILES: C#CCOC(=O)NCC(=O)Nc1cc(NC(=O)[C@H](CCCNC(N)=O)NC(=O)[C@@H](NC(=O)OC(C)(C)C)C(C)C)ccc1COC(=O)N1[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C]

4-((S)-2-((S)-2-((tert-butoxycarbonyl)amino)-3-methylbutanamido)-5-ureidopentanamido)-2-(2-(((prop-2-yn-1-yloxy)carbonyl)amino)acetamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained using the procedure described in Example 3, step 2, however tert-butyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate was replaced with tert-butyl ((S)-3-methyl-1-(((S)-1-((4-((((4-nitrophenoxy)carbonyl)oxy)methyl)-3-(2-(((prop-2-yn-1-yloxy)carbonyl)amino)acetamido)phenyl)amino)-1-oxo-5-ureidopentan-2-yl)amino)-1-oxobutan-2-yl)carbamate (44.6 mg, 0.056 mmoles, 1.0 equiv.). 4-((S)-2-((S)-2-((tert-butoxycarbonyl)amino)-3-methylbutanamido)-5-ureidopentanamido)-2-(2-(((prop-2-yn-1-yloxy)carbonyl)amino)acetamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: (66.5 mg, 0.046 mmoles, 83%). HRMS: MH+=1440.7500 Rt=2.70 min (5 min acidic method-Method D).

### Step 3: Synthesis of 4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(2-(((prop-2-yn-1-yloxy)carbonyl)amino)acetamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

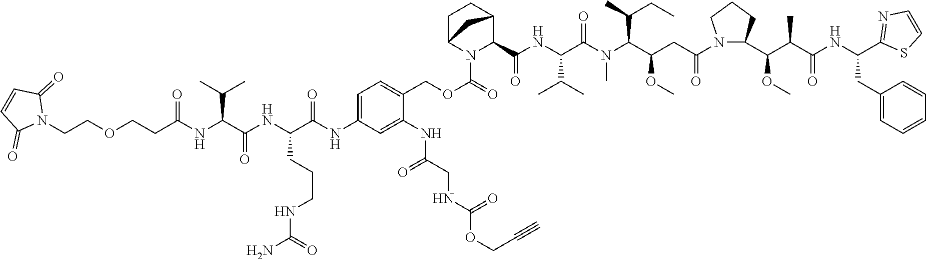 [SMILES: C#CCOC(=O)NCC(=O)Nc1cc(NC(=O)[C@H](CCCNC(N)=O)NC(=O)[C@@H](NC(=O)CCOCCN2C(=O)C=CC2=O)C(C)C)ccc1COC(=O)N1[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C]

4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(2-(((prop-2-yn-1-yloxy)carbonyl)amino)acetamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained using the procedure described in Example 3-1, step 4, however 2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was replaced with 4-((S)-2-((S)-2-((tert-butoxycarbonyl)amino)-3-methylbutanamido)-5-ureidopentanamido)-2-(2-(((prop-2-yn-1-yloxy)carbonyl)amino)acetamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (66.5 mg, 0.046 mmoles, 1.0 equiv.). 4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(2-(((prop-2-yn-1-yloxy)carbonyl)amino)acetamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: (51.4 mg, 0.033 mmoles, 72%). HRMS: MH+=1535.7500 Rt=2.50 min (5 min acidic method-Method D).

### Step 4: Synthesis of 2-(2-((((1-(2,5,8,11,14,17,20,23-octaoxapentacosan-25-yl)-1H-1,2,3-triazol-4-yl)methoxy)carbonyl)amino)acetamido)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L5-P1)

2-(2-((((1-(2,5,8,11,14,17,20,23-octaoxapentacosan-25-yl)-1H-1,2,3-triazol-4-yl)methoxy)carbonyl)amino)acetamido)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L5-P1) was obtained using the procedure described in Example 3-1, step 3, however 4-((S)-2-((S)-2-((tert-butoxycarbonyl)amino)-3-methylbutanamido)-5-ureidopentanamido)-2-((prop-2-yn-1-yloxy)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was replaced with 4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(2-(((prop-2-yn-1-yloxy)carbonyl)amino)acetamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (27.3 mg, 0.018 mmoles, 1.0 equiv.) 2-(2-((((1-(2,5,8,11,14,17,20,23-octaoxapentacosan-25-yl)-1H-1,2,3-triazol-4-yl)methoxy)carbonyl)amino)acetamido)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L5-P1): (18.9 mg, 9.33 μmoles, 52%). HRMS: MH+=1944.9900 Rt=2.45 min (5 min acidic method-Method D).

### Example 3-5: Synthesis of 2-(bis((1-(2,5,8,11,14,17,20,23-octaoxapentacosan-25-yl)-1H-1,2,3-triazol-4-yl)methyl)carbamoyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L6-P1)

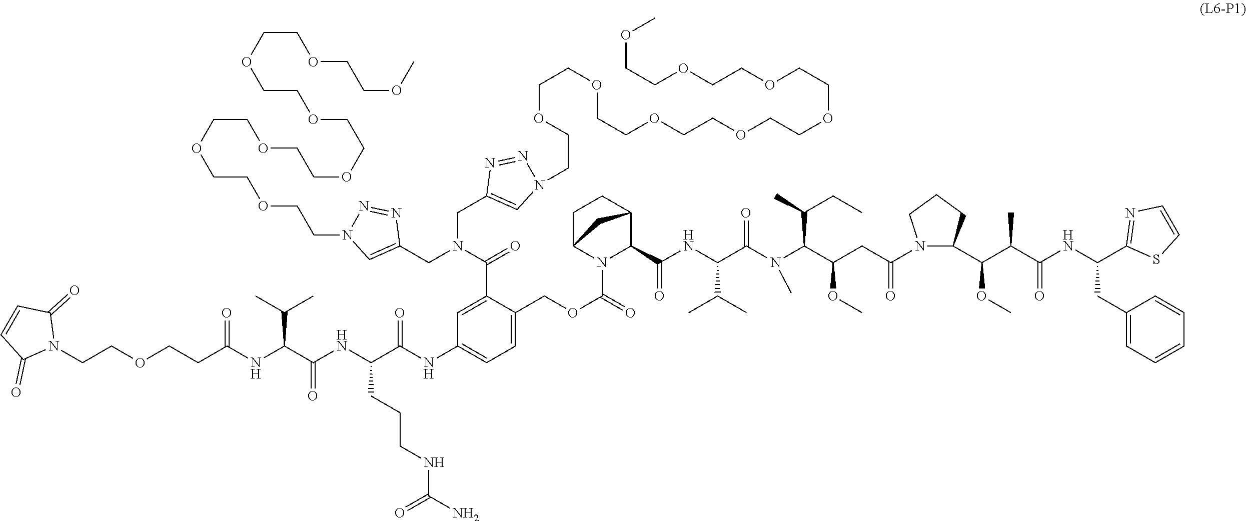 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCOC)nn1)Cc1cn(CCOCCOCCOCCOCCOCCOCCOCCOC)nn1)C(C)C]

### Step 1: Synthesis of tert-butyl ((S)-1-(((S)-1-((3-(di(prop-2-yn-1-yl)carbamoyl)-4-((((4-nitrophenoxy)carbonyl)oxy)methyl)phenyl)amino)-1-oxo-5-ureidopentan-2-yl)amino)-3-methyl-1-oxobutan-2-yl)carbamate

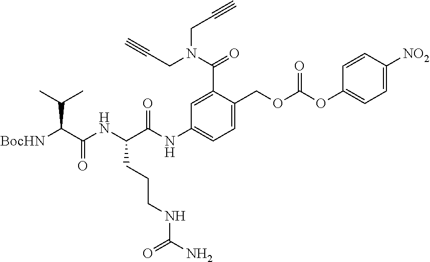 [SMILES: C#CCN(CC#C)C(=O)c1cc(NC(=O)[C@H](CCCNC(N)=O)NC(=O)[C@@H](NC(=O)OC(C)(C)C)C(C)C)ccc1COC(=O)Oc1ccc([N+](=O)[O-])cc1]

Tert-butyl ((S)-1-(((S)-1-((3-(di(prop-2-yn-1-yl)carbamoyl)-4-((((4-nitrophenoxy)carbonyl)oxy)methyl)phenyl)amino)-1-oxo-5-ureidopentan-2-yl)amino)-3-methyl-1-oxobutan-2-yl)carbamate was obtained using the procedure described in Example 3-1, step 1, however tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (LI-1) was replaced with tert-butyl ((S)-1-(((S)-1-((3-(di(prop-2-yn-1-yl)carbamoyl)-4-(hydroxymethyl)phenyl)amino)-1-oxo-5-ureidopentan-2-yl)amino)-3-methyl-1-oxobutan-2-yl)carbamate (LI-5) (1.51 g, 2.52 mmoles, 1.0 equiv.), and N,N-diisopropylethylamine was omitted.

Tert-butyl ((S)-1-(((S)-1-((3-(di(prop-2-yn-1-yl)carbamoyl)-4-((((4-nitrophenoxy)carbonyl)oxy)methyl)phenyl)amino)-1-oxo-5-ureidopentan-2-yl)amino)-3-methyl-1-oxobutan-2-yl)carbamate: (1.33 g, 1.741 mmoles, 69%). LCMS: MH+764.3, Rt=1.00 min (2 min acidic method-Method A).

### Step 2: Synthesis of 4-((S)-2-((S)-2-((tert-butoxycarbonyl)amino)-3-methylbutanamido)-5-ureidopentanamido)-2-(di(prop-2-yn-1-yl)carbamoyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

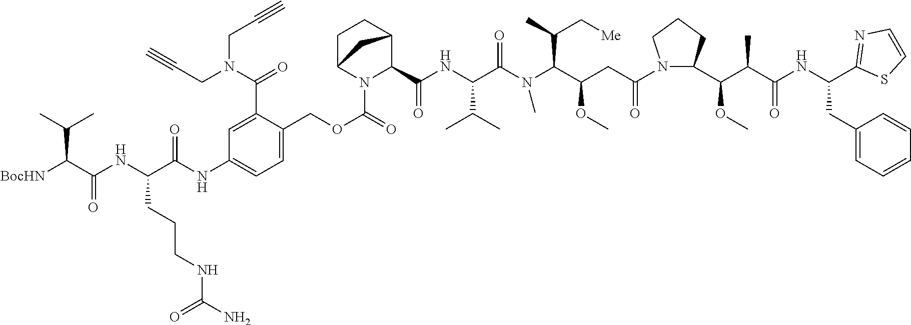 [SMILES: C#CCN(CC#C)C(=O)c1cc(NC(=O)[C@H](CCCNC(N)=O)NC(=O)[C@@H](NC(=O)OC(C)(C)C)C(C)C)ccc1COC(=O)N1[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C]

4-((S)-2-((S)-2-((tert-butoxycarbonyl)amino)-3-methylbutanamido)-5-ureidopentanamido)-2-(di(prop-2-yn-1-yl)carbamoyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained using the procedure described in Example 3, step 2, however tert-butyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate was replaced with tert-butyl ((S)-1-(((S)-1-((3-(di(prop-2-yn-1-yl)carbamoyl)-4-((((4-nitrophenoxy)carbonyl)oxy)methyl)phenyl)amino)-1-oxo-5-ureidopentan-2-yl)amino)-3-methyl-1-oxobutan-2-yl)carbamate (42.7 mg, 0.056 mmoles, 1.0 equiv.). 4-((S)-2-((S)-2-((tert-butoxycarbonyl)amino)-3-methylbutanamido)-5-ureidopentanamido)-2-(di(prop-2-yn-1-yl)carbamoyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: (50.9 mg, 0.036 mmoles, 64%). LCMS: MH+1406.0, Rt=1.14 min (2 min basic method-Method B).

### Step 3: Synthesis of 2-(bis((1-(2,5,8,11,14,17,20,23-octaoxapentacosan-25-yl)-1H-1,2,3-triazol-4-yl)methyl)carbamoyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

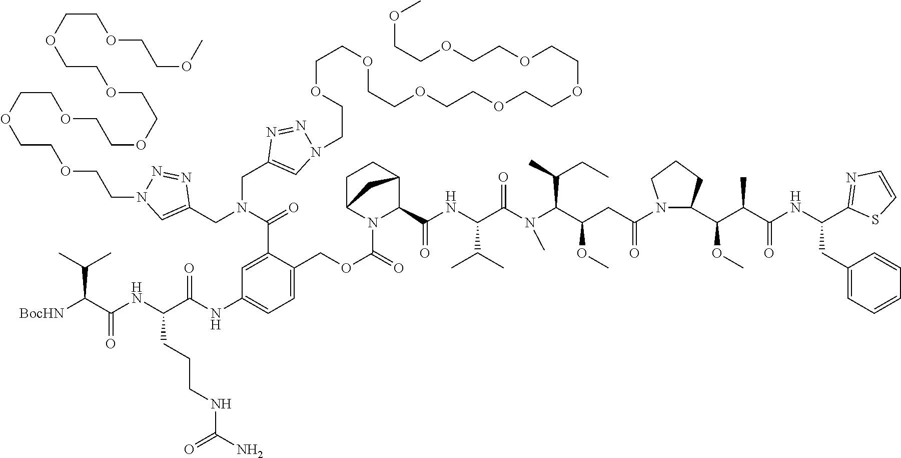 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)OC(C)(C)C)C(C)C)cc1C(=O)N(Cc1cn(CCOCCOCCOCCOCCOCCOCCOCCOC)nn1)Cc1cn(CCOCCOCCOCCOCCOCCOCCOCCOC)nn1)C(C)C]

2-(bis((1-(2,5,8,11,14,17,20,23-octaoxapentacosan-25-yl)-1H-1,2,3-triazol-4-yl)methyl)carbamoyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained using the procedure described in Example 3-1, step 3, however 4-((S)-2-((S)-2-((tert-butoxycarbonyl)amino)-3-methylbutanamido)-5-ureidopentanamido)-2-((prop-2-yn-1-yloxy)methyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was replaced with 4-((S)-2-((S)-2-((tert-butoxycarbonyl)amino)-3-methylbutanamido)-5-ureidopentanamido)-2-(di(prop-2-yn-1-yl)carbamoyl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (50 mg, 0.036 mmoles, 1.0 equiv.). 2-(bis((1-(2,5,8,11,14,17,20,23-octaoxapentacosan-25-yl)-1H-1,2,3-triazol-4-yl)methyl)carbamoyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino) propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: (79 mg, 0.036 mmoles, 99%). LCMS: [M+2H]2+1112.8, Rt=1.04 min (2 min acidic method-Method A).

### Step 4: Synthesis of 2-(bis((1-(2,5,8,11,14,17,20,23-octaoxapentacosan-25-yl)-1H-1,2,3-triazol-4-yl)methyl)carbamoyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L6-P1)

2-(bis((1-(2,5,8,11,14,17,20,23-octaoxapentacosan-25-yl)-1H-1,2,3-triazol-4-yl)methyl)carbamoyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L6-P1) was obtained using the procedure described in Example 3-1, step 4, however 2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was replaced with 2-(bis((1-(2,5,8,11,14,17,20,23-octaoxapentacosan-25-yl)-1H-1,2,3-triazol-4-yl)methyl)carbamoyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (79 mg, 0.036 mmoles, 1.0 equiv.). 2-(bis((1-(2,5,8,11,14,17,20,23-octaoxapentacosan-25-yl)-1H-1,2,3-triazol-4-yl)methyl)carbamoyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L6-P1) (8.1 mg, 3.32 μmoles, 9%). HRMS: MH+=2319.2450 Rt=2.47 min (5 min acidic method-Method D).

### Example 3-6: Synthesis of (1R,3S,4S)-2-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium (L137-P2) or (1R,3S,4S)-2-(2-(((1-(38-carboxy-3,6,9,12,15,18,21,24,27,30,33,36-dodecaoxaoctatriacontyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium (L140-P2)

### Step 1: Synthesis of tert-butyl ((S)-1-(((S)-1-((4-(chloromethyl)-3-((prop-2-yn-1-yloxy)methyl)phenyl)amino)-1-oxo-5-ureidopentan-2-yl)amino)-3-methyl-1-oxobutan-2-yl)carbamate

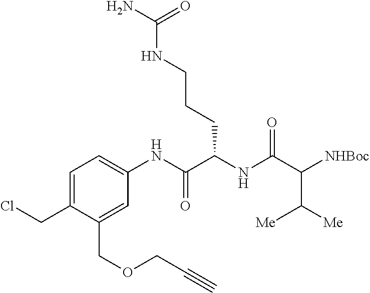 [SMILES: C#CCOCc1cc(NC(=O)[C@H](CCCNC(N)=O)NC(=O)C(NC(=O)OC(C)(C)C)C(C)C)ccc1CCl]

To a solution of tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (2.00 grams, 3.65 mmol, 1.0 equiv.) in acetonitrile (13.3 mL) at 0° C. was added thionyl chloride (0.53 mL, 7.30 mmol, 2.0 equiv). After stirring in the ice bath for one hour the solution was diluted with water (40 mL) and the resulting white precipitate was collected by filtration, air drying and drying under high vacuum to yield tert-butyl ((S)-1-(((S)-1-((4-(chloromethyl)-3-((prop-2-yn-1-yloxy)methyl)phenyl)amino)-1-oxo-5-ureidopentan-2-yl)amino)-3-methyl-1-oxobutan-2-yl)carbamate. LCMS: MNa+588.5; Rt=2.17 min (5 min acidic method).

### Step 2: Synthesis of (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate

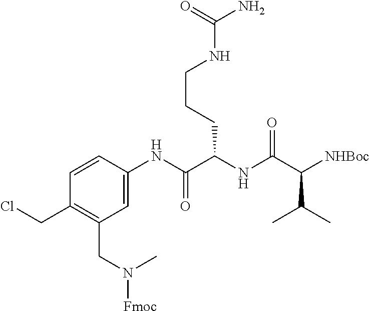

To (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (200 mg, 0.269 mmoles, 1.0 equiv.) in CH₂Cl₂ (10 mL) was added pyridine (0.130 mL, 1.61 mmoles, 6 equiv.). The heterogeneous mixture was cooled in a 0° C. ice bath and thionyl chloride (0.059 mL, 0.806 mmoles, 3 equiv.). After stirring in the ice bath briefly the reaction was stirred as it warmed up to room temperature for 2 hours. The reaction was purified by ISCO SiO₂ chromatography (0-30% MeOH/CH₂Cl₂) and (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate was obtained. LCMS: MH+=763.2; Rt=1.18 min (2 min acidic method).

### Step 3: Synthesis of (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide (P2)

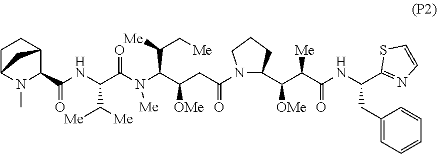 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C)C(C)C]

(1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide can be treated with paraformaldehyde under standard reductive amination conditions to yield (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide.

### Step 4: Syntheses of L137-P2, L140-P2, and Other Compounds in Table 4C can be Made According to One of the Following Schemes

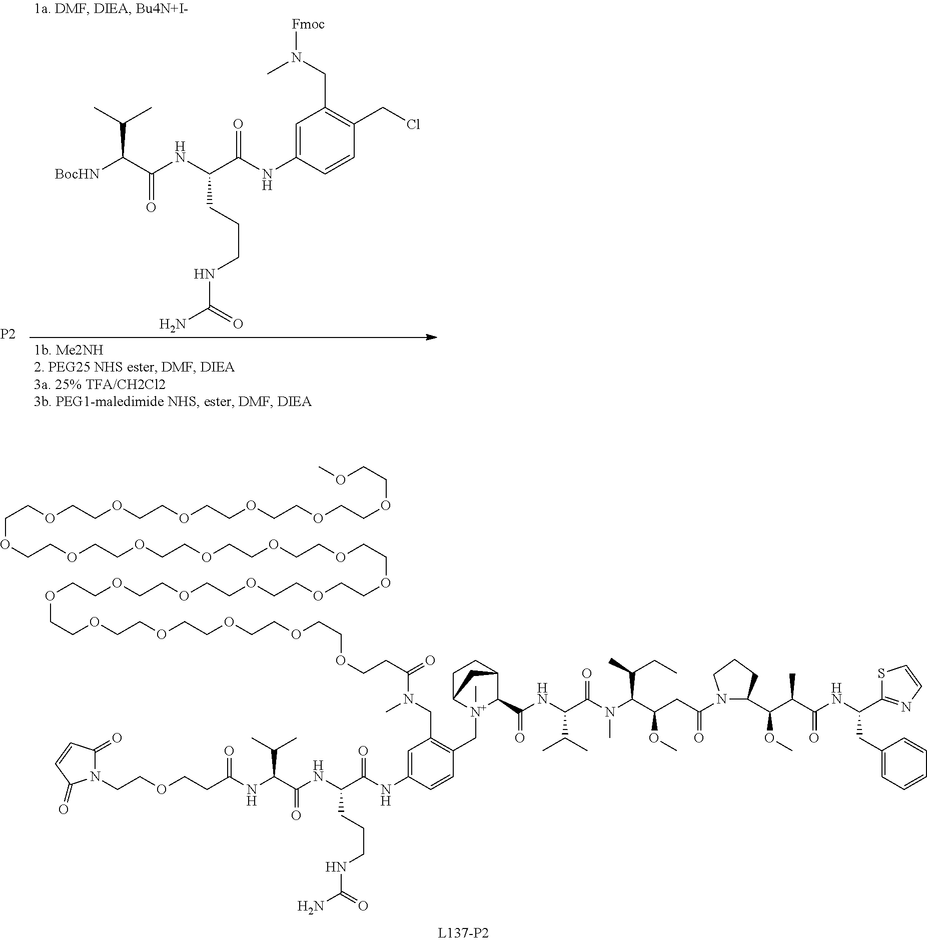

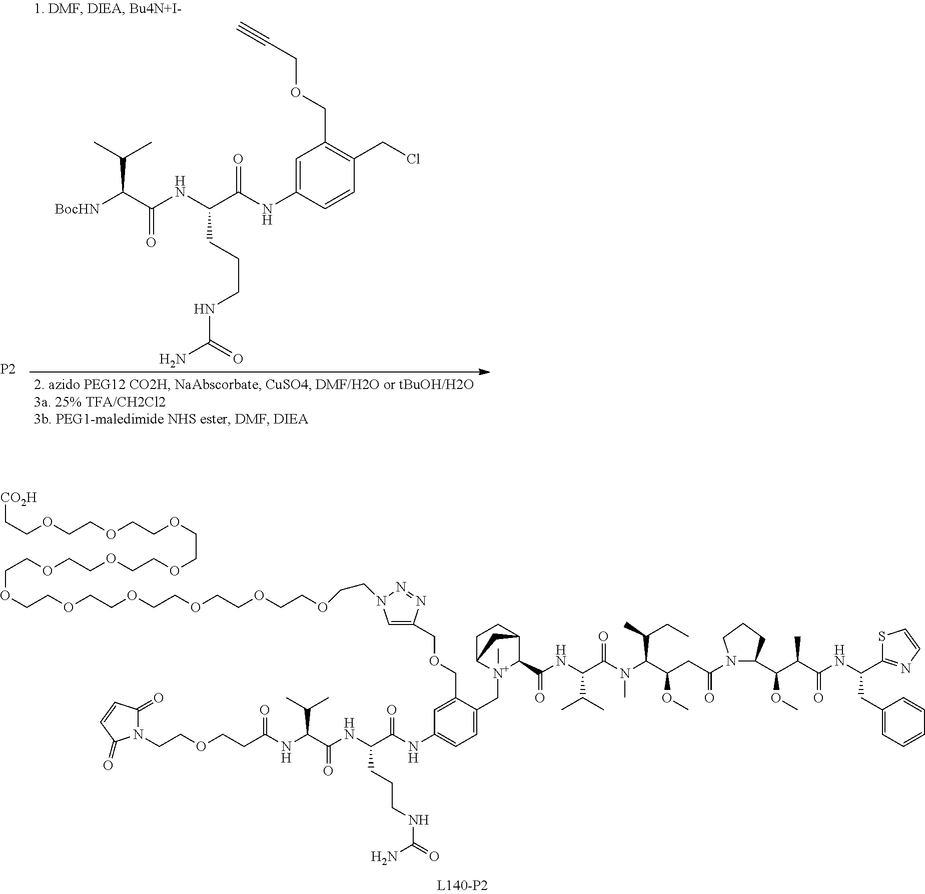 [SMILES: C#CCOCc1cc(NC(=O)[C@H](CCCNC(N)=O)NC(=O)[C@@H](NC(=O)OC(C)(C)C)C(C)C)ccc1CCl ; "P2" ; CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCOCCOCCOCCOCCC(=O)O)nn1)C(C)C]

Examples of additional Linker-Drug compounds that can be synthesized using the methods described in Example 3-1 to 3-6 are given in Tables 4A-4C below.

TABLE 4A

| Com- | |
|---|---|
| pound | |
| Num- | |
| ber | |
| L7- P1 | 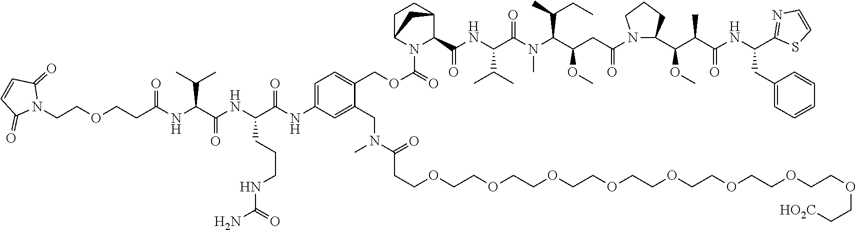 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCC(=O)O)C(C)C] |
| L8- P1 | 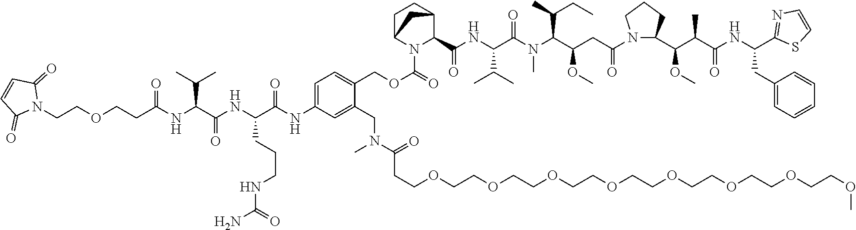 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOC)C(C)C] |
| L9- P1 | 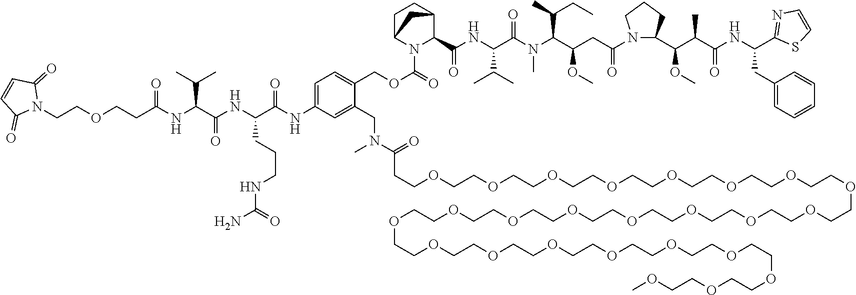 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCOC)C(C)C] |
| L10- P1 | 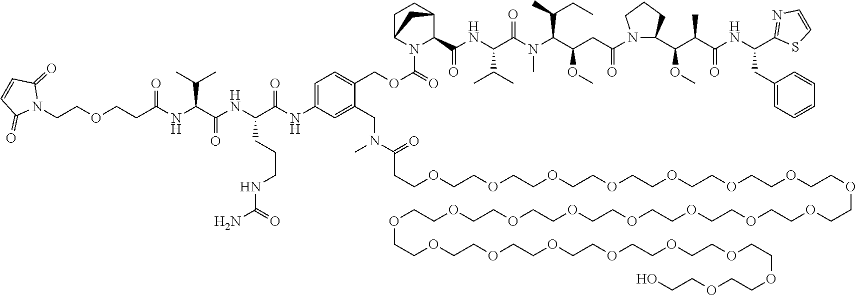 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCO)C(C)C] |
| L11- P1 | 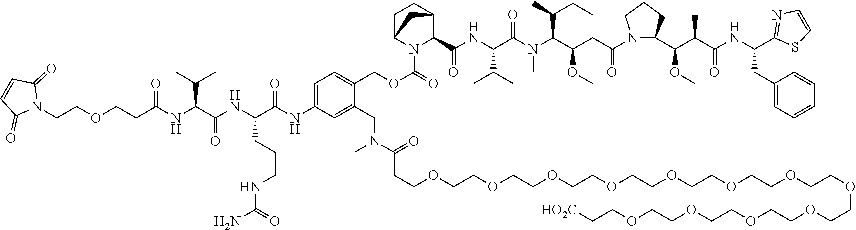 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCC(=O)O)C(C)C] |
| L12- P1 | 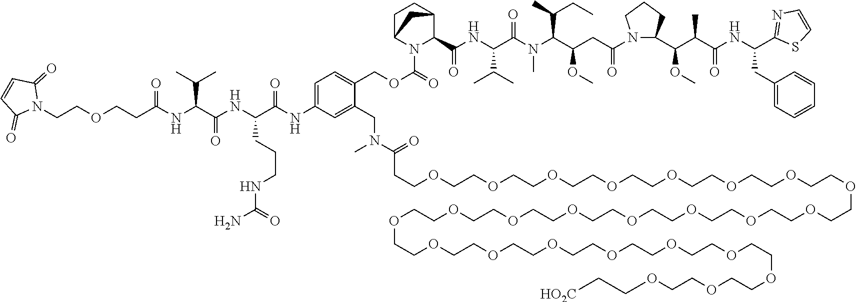 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)C(C)C] |
| L13- P1 | 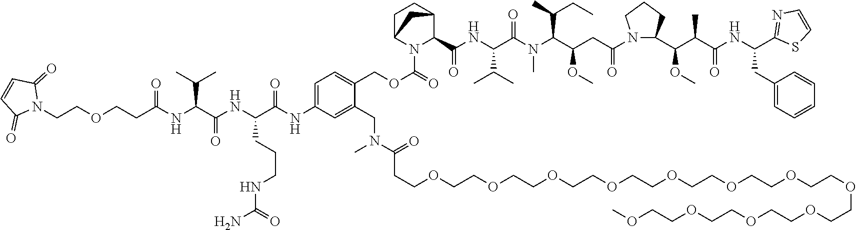 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOC)C(C)C] |
| L14- P1 | 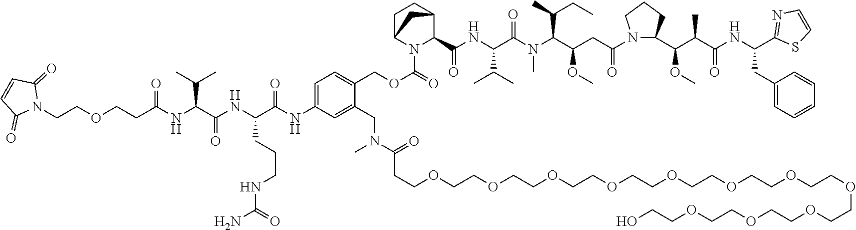 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCO)C(C)C] |
| L15- P1 | 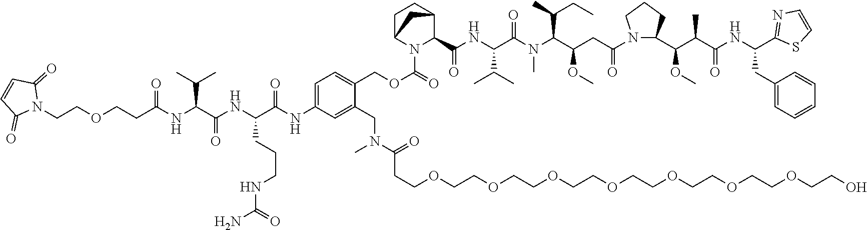 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCO)C(C)C] |
| L16- P1 | 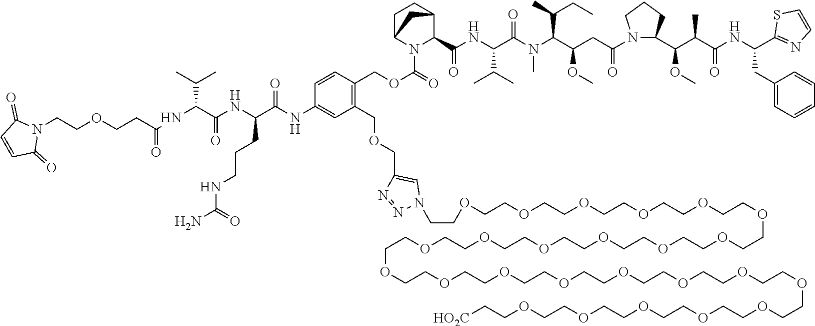 |
| L17- P1 | 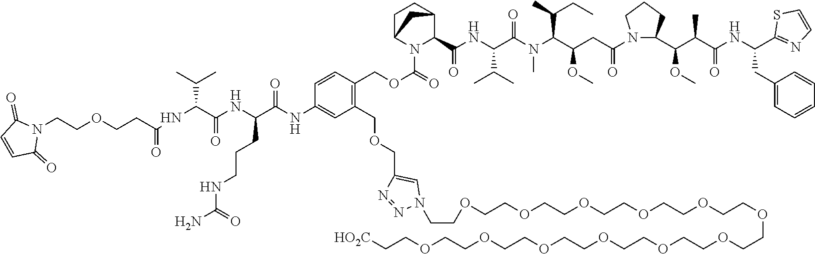 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@@H](CCCNC(N)=O)NC(=O)[C@H](NC(=O)CCOCCN2C(=O)C=CC2=O)C(C)C)cc1COCc1cn(CCOCCOCCOCCOCCOCCOCCOCCOCCOCCOCCOCCOCCC(=O)O)nn1)C(C)C] |
| L18- P1 | 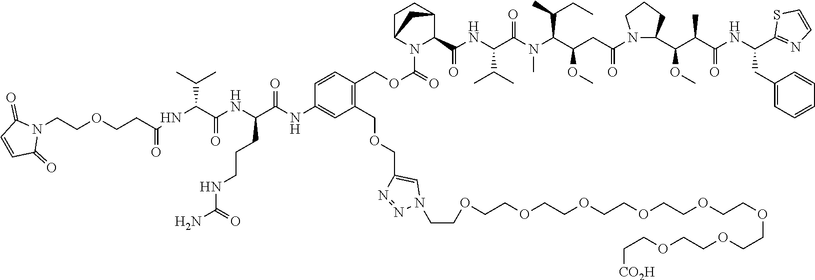 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@@H](CCCNC(N)=O)NC(=O)[C@H](NC(=O)CCOCCN2C(=O)C=CC2=O)C(C)C)cc1COCc1cn(CCOCCOCCOCCOCCOCCOCCOCCOCCC(=O)O)nn1)C(C)C] |
| L19- P1 | 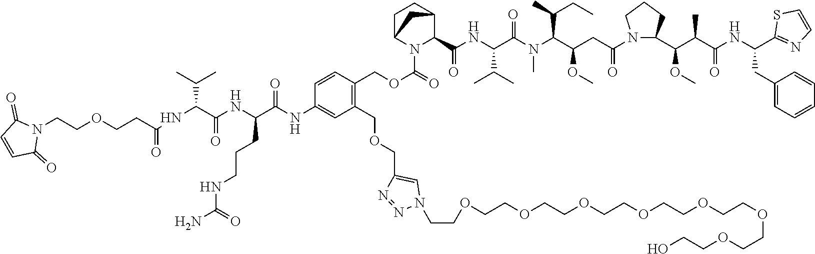 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@@H](CCCNC(N)=O)NC(=O)[C@H](NC(=O)CCOCCN2C(=O)C=CC2=O)C(C)C)cc1COCc1cn(CCOCCOCCOCCOCCOCCOCCOCCO)nn1)C(C)C] |
| L20- P1 | 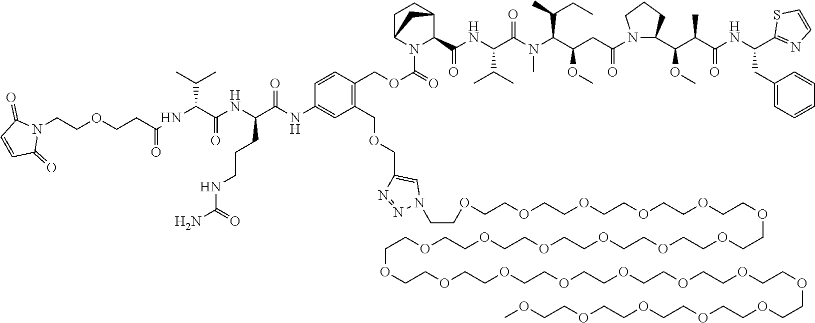 |
| L21- P1 | 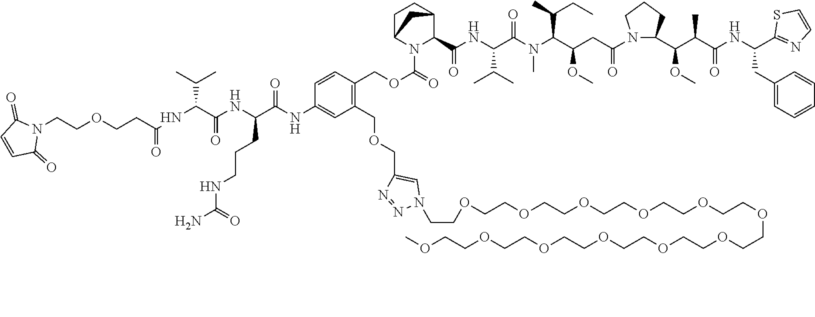 |
| L22- P1 | 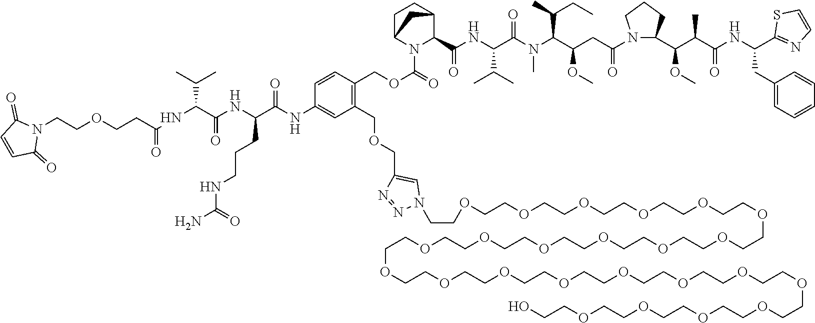 |
| L23- P1 | 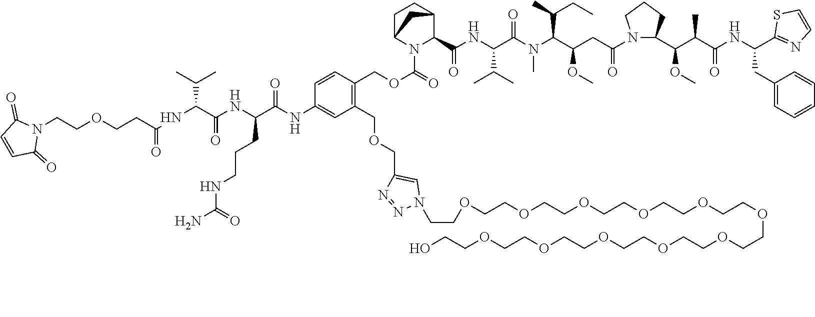 |
| L24- P1 | 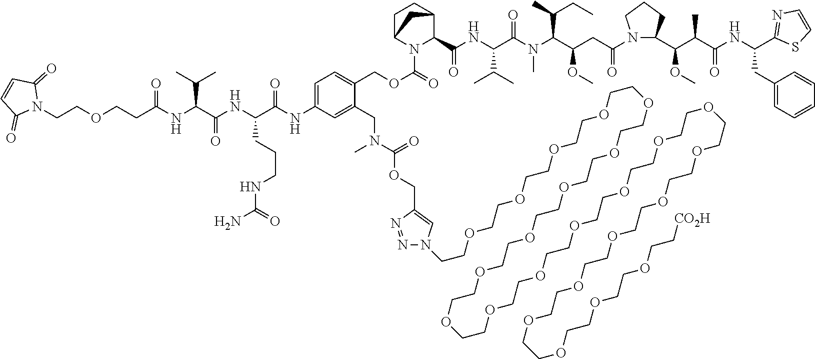 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCOCCOCCOCCOCCOCCOCCOCCOCCOCCOCCOCCOCCOCCOCCOCCOCCC(=O)O)nn1)C(C)C] |
| L25- P1 | 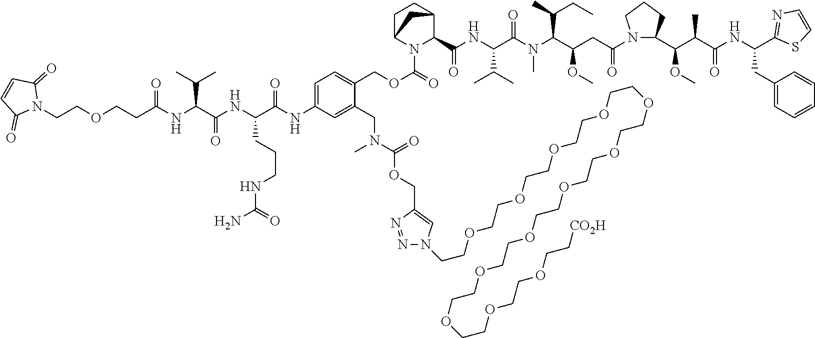 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCOCCOCCOCCOCCC(=O)O)nn1)C(C)C] |
| L26- P1 | 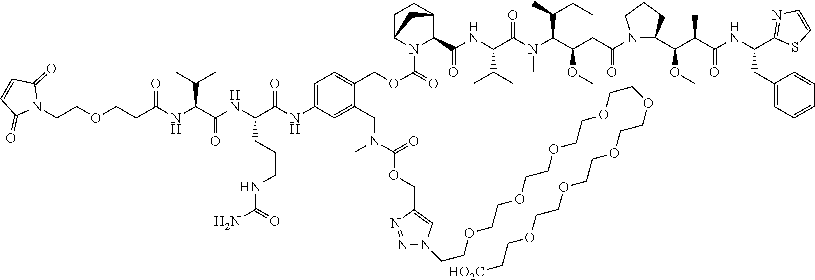 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCC(=O)O)nn1)C(C)C] |
| L27- P1 | 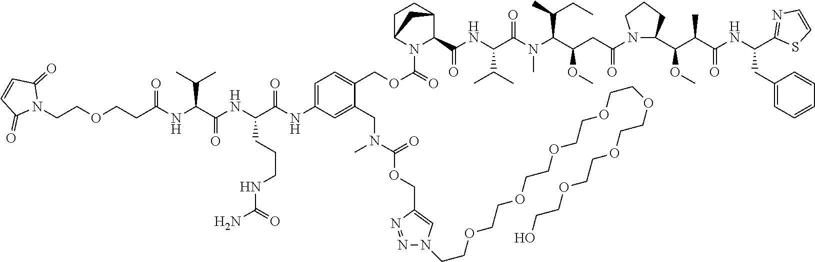 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCO)nn1)C(C)C] |
| L28- P1 | 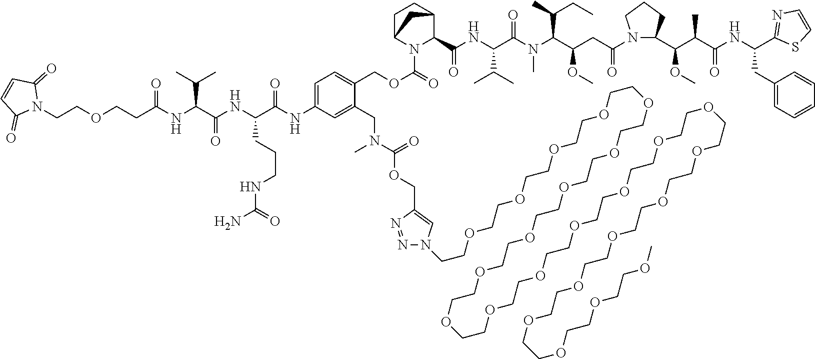 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCOCCOCCOCCOCCOCCOCCOCCOCCOCCOCCOCCOCCOCCOCCOCCOC)nn1)C(C)C] |
| L29- P1 | 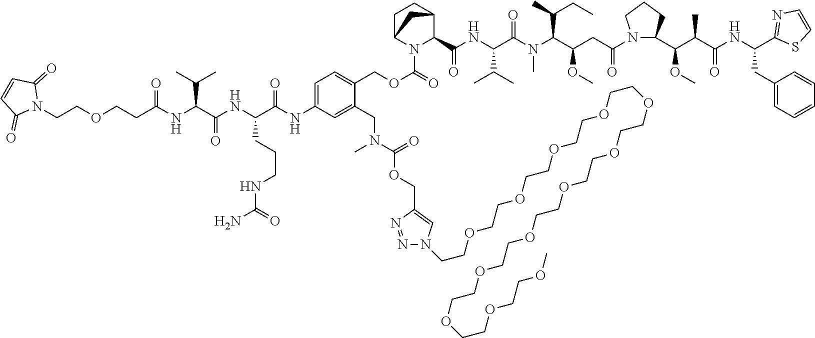 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCOCCOCCOCCOC)nn1)C(C)C] |
| L30- P1 | 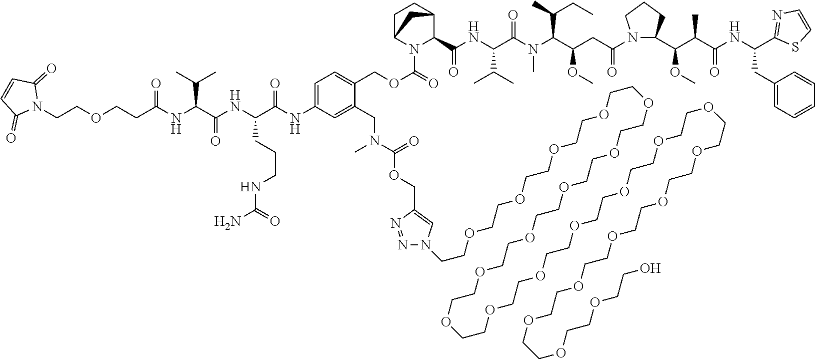 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCOCCOCCOCCOCCOCCOCCOCCOCCOCCOCCOCCOCCOCCOCCOCCO)nn1)C(C)C] |
| L31- P1 | 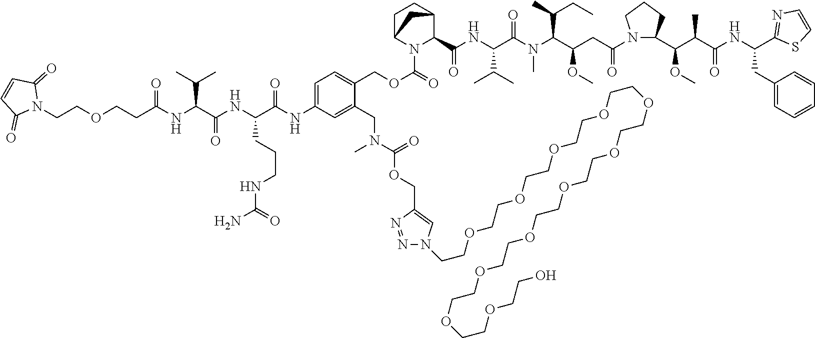 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCOCCOCCOCCO)nn1)C(C)C] |
| L32- P1 | 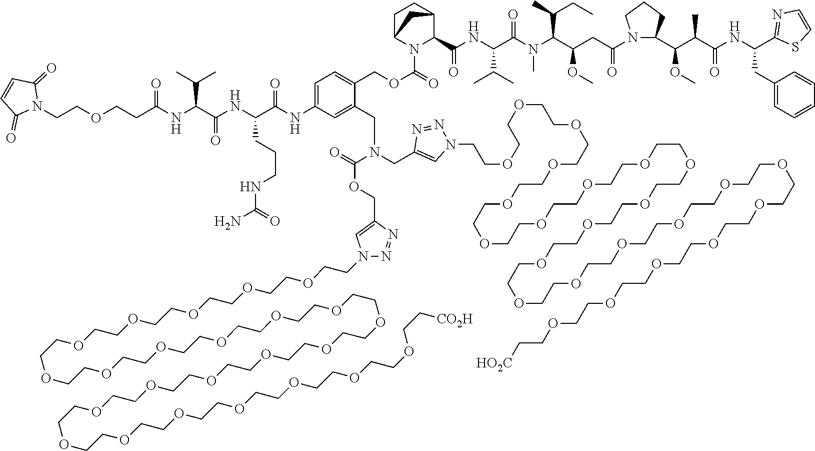 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCOCCOCCOCCOCCOCCOCCOCCOCCOCCOCCOCCOCCOCCOCCOCCOCCC(=O)O)nn1)C(=O)OCc1cn(CCOCCOCCOCCOCCOCCOCCOCCOCCOCCOCCOCCOCCOCCOCCOCCOCCOCCOCCOCCOCCOCCOCCOCCOCCC(=O)O)nn1)C(C)C] |
| L33- P1 | 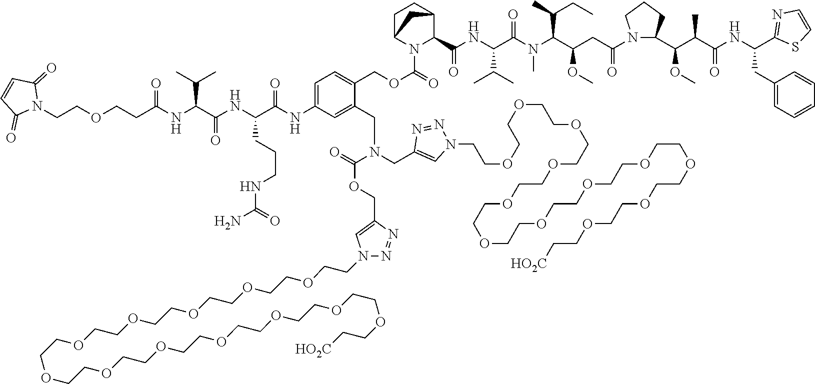 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCOCCOCCOCCOCCC(=O)O)nn1)C(=O)OCc1cn(CCOCCOCCOCCOCCOCCOCCOCCOCCOCCOCCOCCOCCC(=O)O)nn1)C(C)C] |
| L34- P1 | 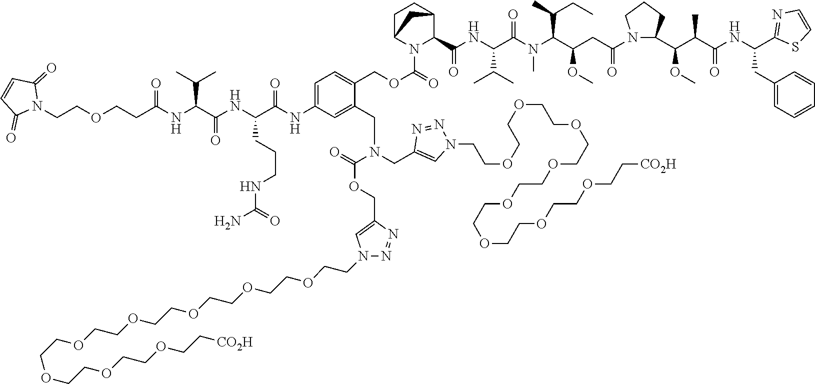 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCC(=O)O)nn1)C(=O)OCc1cn(CCOCCOCCOCCOCCOCCOCCOCCOCCC(=O)O)nn1)C(C)C] |
| L35- P1 | 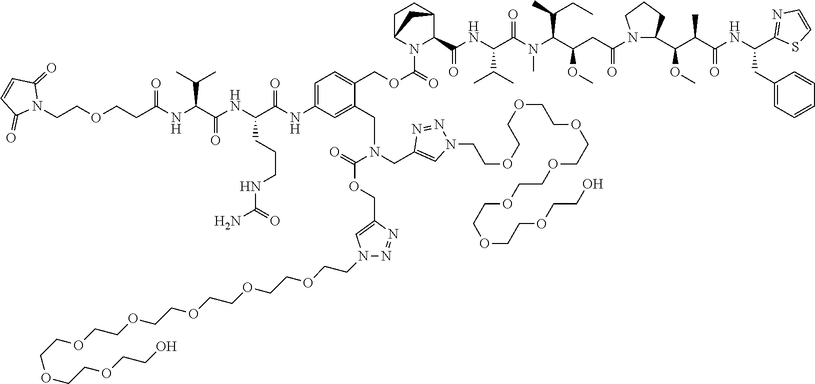 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCO)nn1)C(=O)OCc1cn(CCOCCOCCOCCOCCOCCOCCOCCO)nn1)C(C)C] |
| L36- P1 | 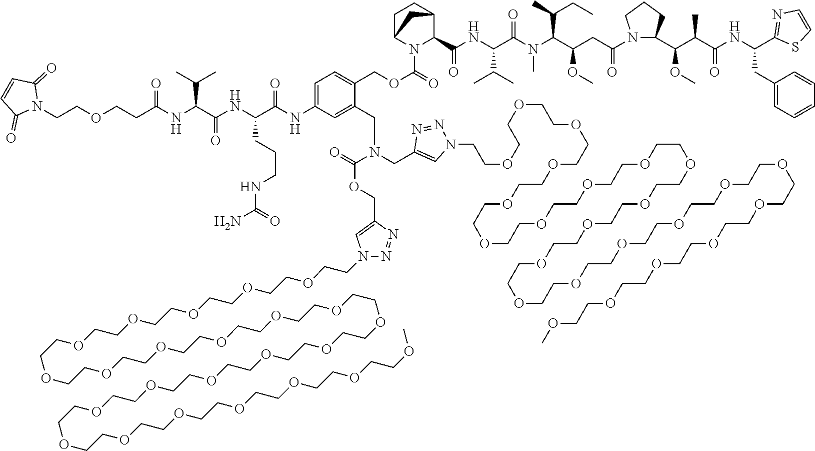 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCOCCOCCOCCOCCOCCOCCOCCOCCOCCOCCOCCOCCOCCOCCOCCOC)nn1)C(=O)OCc1cn(CCOCCOCCOCCOCCOCCOCCOCCOCCOCCOCCOCCOCCOCCOCCOCCOCCOCCOCCOCCOCCOCCOCCOCCOC)nn1)C(C)C] |
| L37- P1 | 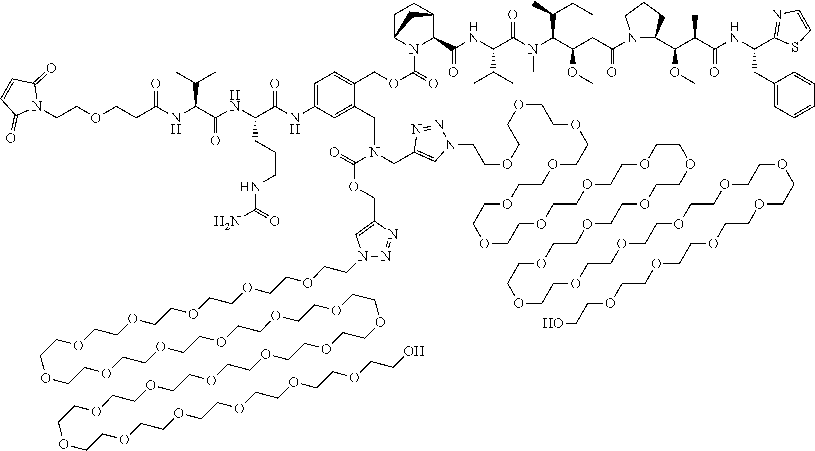 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCOCCOCCOCCOCCOCCOCCOCCOCCOCCOCCOCCOCCOCCOCCOCCO)nn1)C(=O)OCc1cn(CCOCCOCCOCCOCCOCCOCCOCCOCCOCCOCCOCCOCCOCCOCCOCCOCCOCCOCCOCCOCCOCCOCCOCCO)nn1)C(C)C] |
| L38- P1 | 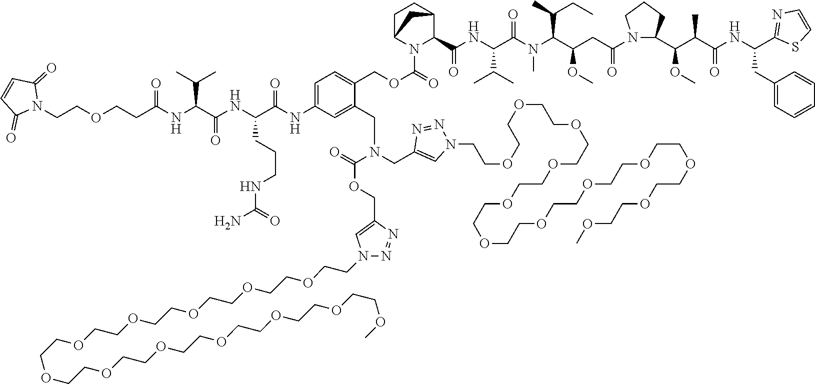 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCOCCOCCOCCOC)nn1)C(=O)OCc1cn(CCOCCOCCOCCOCCOCCOCCOCCOCCOCCOCCOCCOC)nn1)C(C)C] |
| L39- P1 | 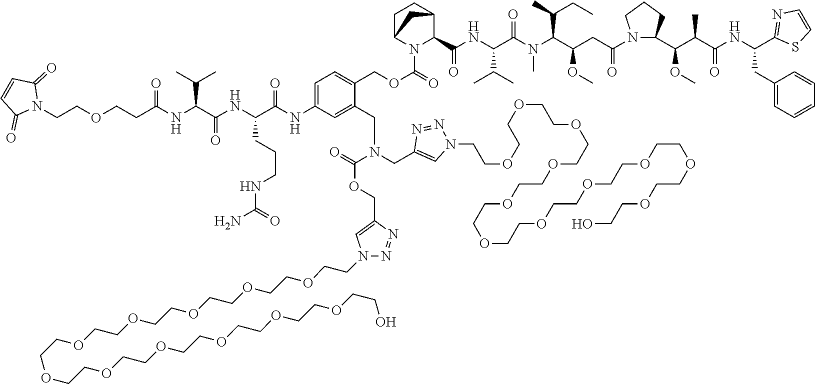 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCOCCOCCOCCO)nn1)C(=O)OCc1cn(CCOCCOCCOCCOCCOCCOCCOCCOCCOCCOCCOCCO)nn1)C(C)C] |
| L40- P1 | 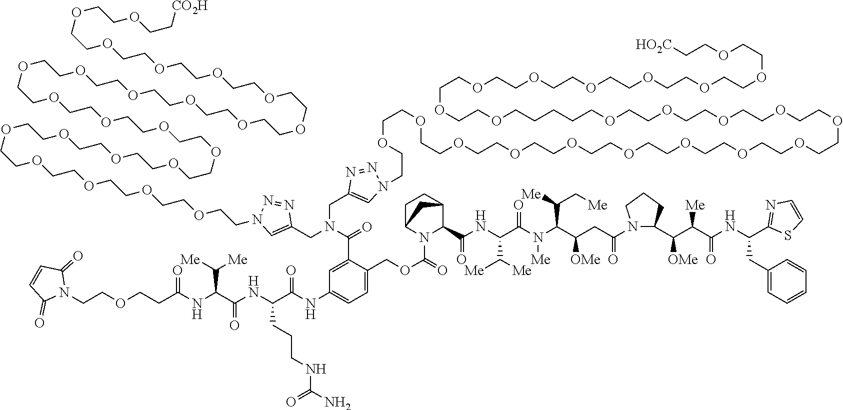 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCOCCOCCOCCOCCOCCOCCOCCCCCOCCOCCOCCOCCOCCOCCOCCOCCOCCC(=O)O)nn1)Cc1cn(CCOCCOCCOCCOCCOCCOCCOCCOCCOCCOCCOCCOCCOCCOCCOCCOCCOCCOCCOCCOCCOCCOCCOCCOCCC(=O)O)nn1)C(C)C] |
| L41- P1 | 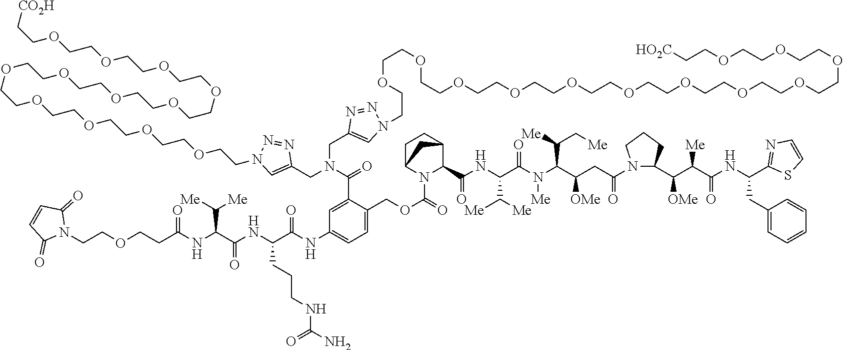 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCOCCOCCOCCOCCOCCC(=O)O)nn1)Cc1cn(CCOCCOCCOCCOCCOCCOCCOCCOCCOCCOCCOCCOCCC(=O)O)nn1)C(C)C] |
| L42- P1 | 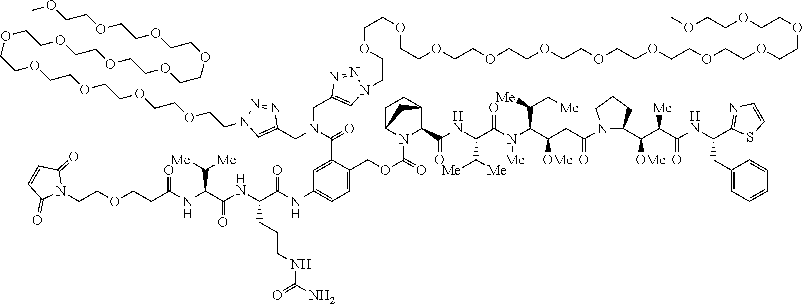 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCOCCOCCOCCOCCOC)nn1)Cc1cn(CCOCCOCCOCCOCCOCCOCCOCCOCCOCCOCCOCCOC)nn1)C(C)C] |
| L43- P1 | 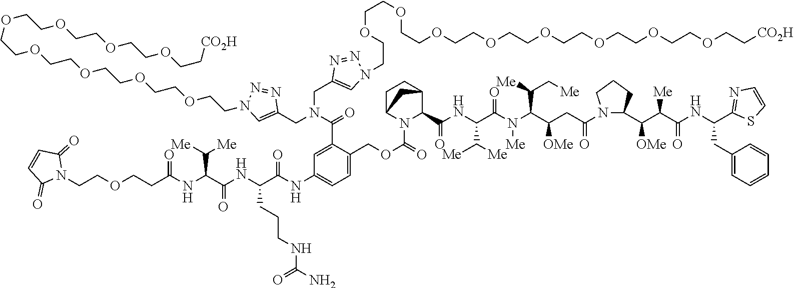 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCOCCC(=O)O)nn1)Cc1cn(CCOCCOCCOCCOCCOCCOCCOCCOCCC(=O)O)nn1)C(C)C] |
| L44- P1 | 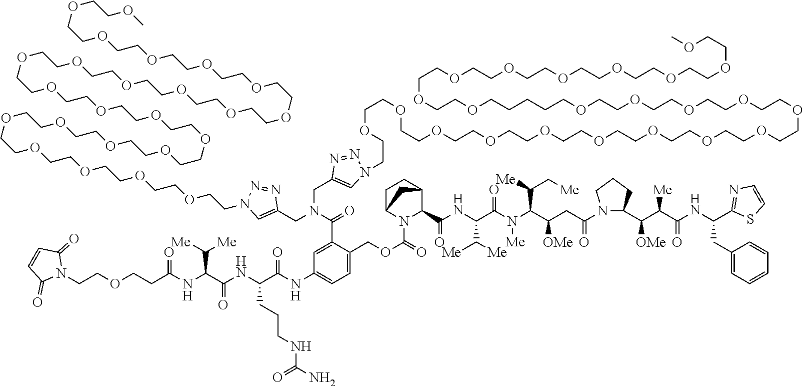 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCOCCOCCOCCOCCOCCOCCOCCCCCOCCOCCOCCOCCOCCOCCOCCOCCOC)nn1)Cc1cn(CCOCCOCCOCCOCCOCCOCCOCCOCCOCCOCCOCCOCCOCCOCCOCCOCCOCCOCCOCCOCCOCCOCCOCCOC)nn1)C(C)C] |
| L45- P1 | 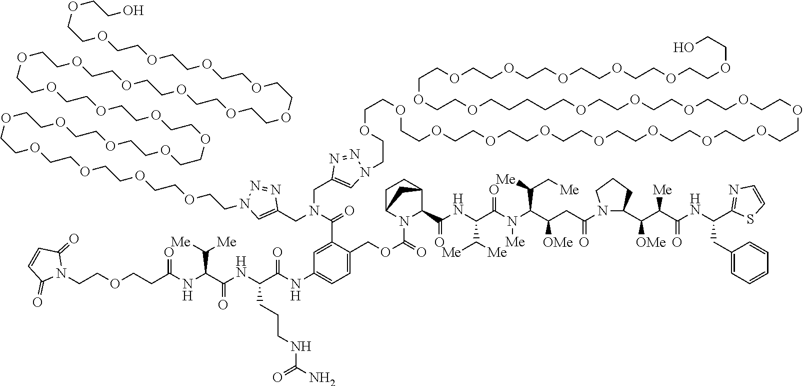 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCOCCOCCOCCOCCOCCOCCOCCCCCOCCOCCOCCOCCOCCOCCOCCOCCO)nn1)Cc1cn(CCOCCOCCOCCOCCOCCOCCOCCOCCOCCOCCOCCOCCOCCOCCOCCOCCOCCOCCOCCOCCOCCOCCOCCO)nn1)C(C)C] |
| L46- P1 | 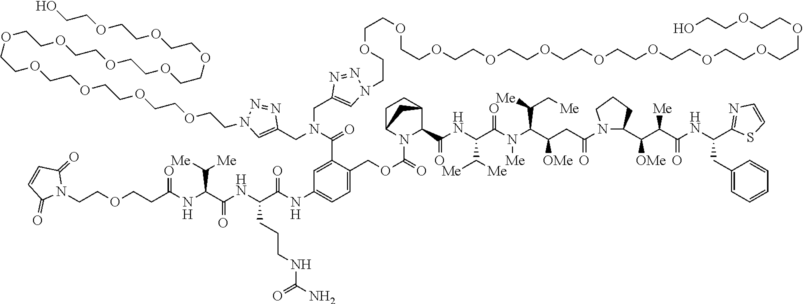 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCOCCOCCOCCOCCO)nn1)Cc1cn(CCOCCOCCOCCOCCOCCOCCOCCOCCOCCOCCOCCO)nn1)C(C)C] |
| L47- P1 | 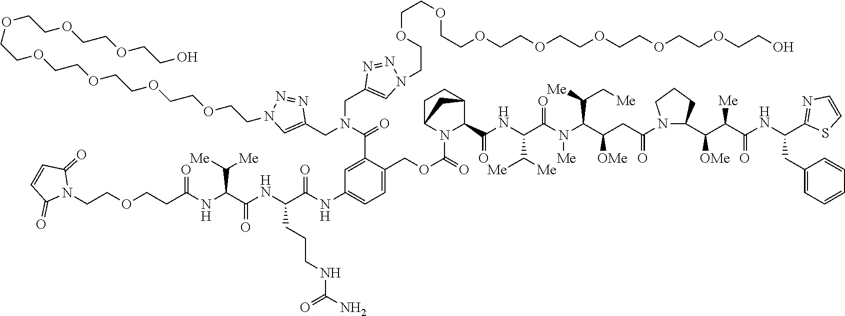 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCO)nn1)Cc1cn(CCOCCOCCOCCOCCOCCOCCOCCO)nn1)C(C)C] |
| L48- P1 | 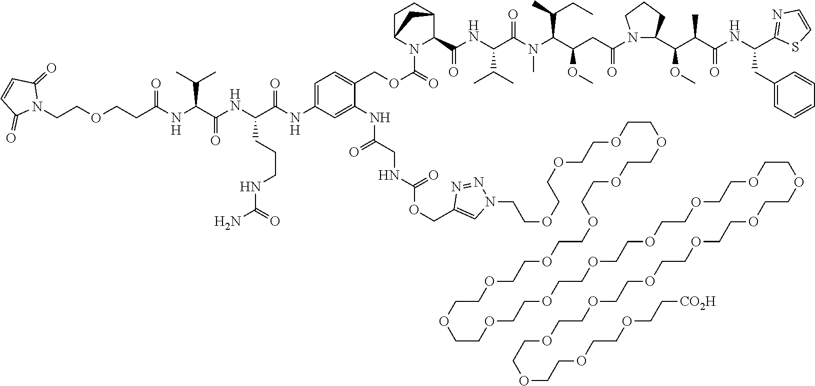 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCOCCOCCOCCOCCOCCOCCOCCOCCOCCOCCOCCOCCOCCOCCOCCOCCOCCC(=O)O)nn1)C(C)C] |
| L49- P1 | 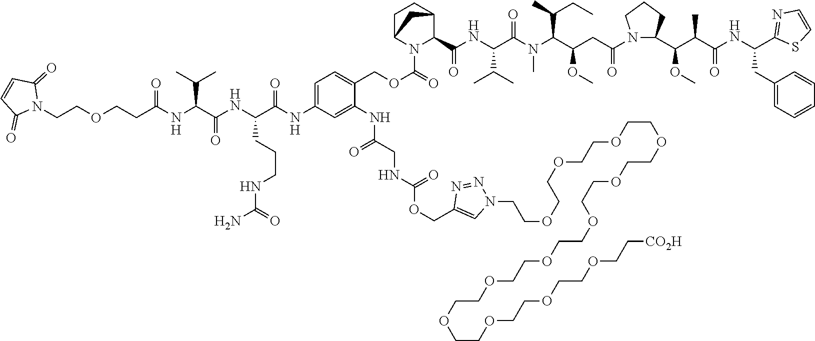 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCOCCOCCOCCOCCOCCC(=O)O)nn1)C(C)C] |
| L50- P1 | 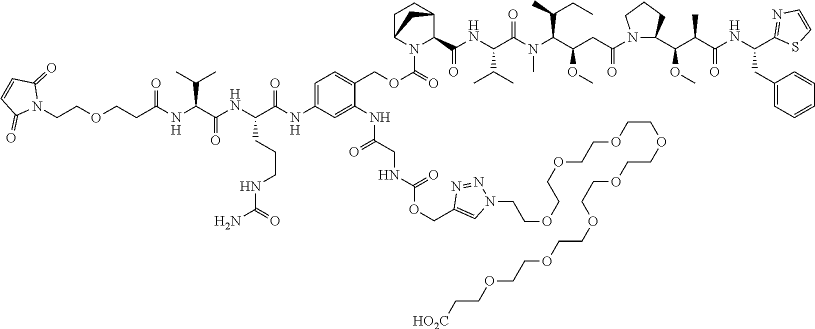 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCOCCC(=O)O)nn1)C(C)C] |
| L51- P1 | 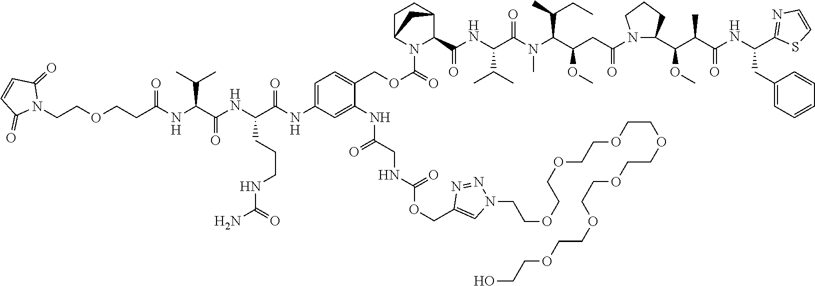 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCO)nn1)C(C)C] |
| L52- P1 | 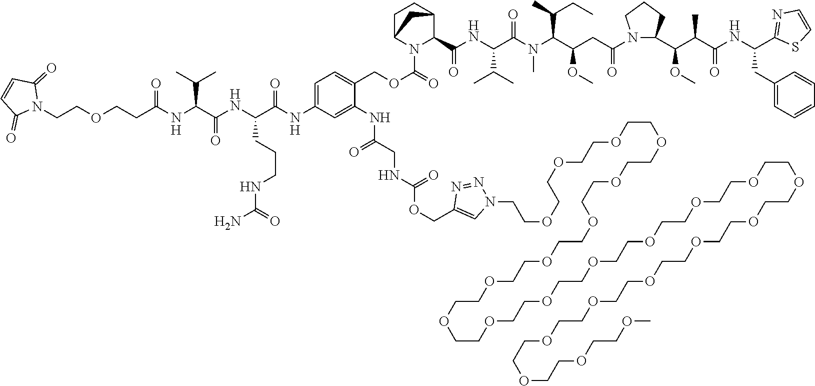 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCOCCOCCOCCOCCOCCOCCOCCOCCOCCOCCOCCOCCOCCOCCOCCOCCOC)nn1)C(C)C] |
| L53- P1 | 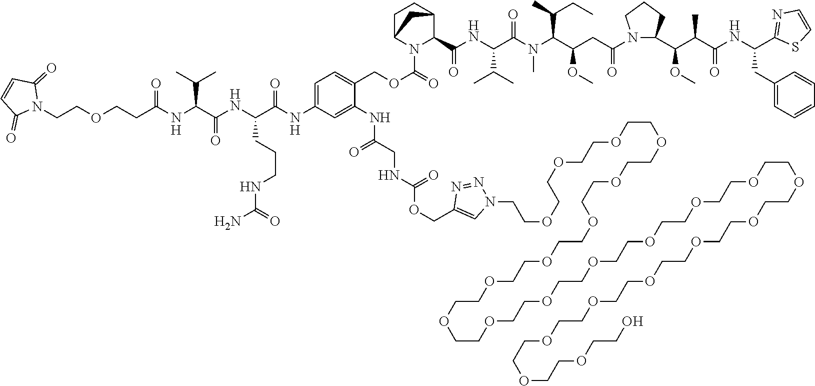 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCOCCOCCOCCOCCOCCOCCOCCOCCOCCOCCOCCOCCOCCOCCOCCOCCO)nn1)C(C)C] |
| L54- P1 | 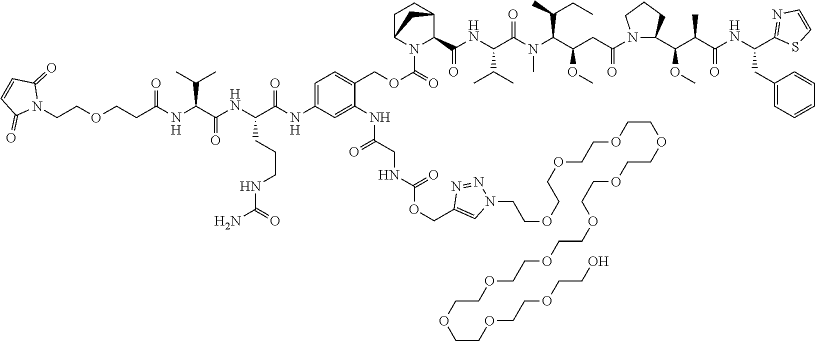 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCOCCOCCOCCOCCO)nn1)C(C)C] |
| L55- P1 | 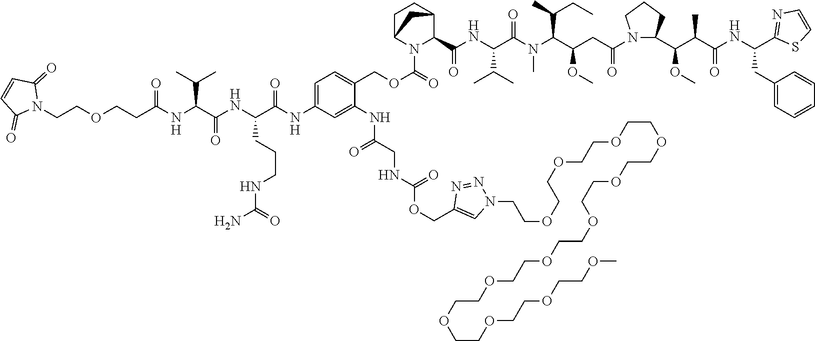 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCOCCOCCOCCOCCOC)nn1)C(C)C] |
| L56- P1 | 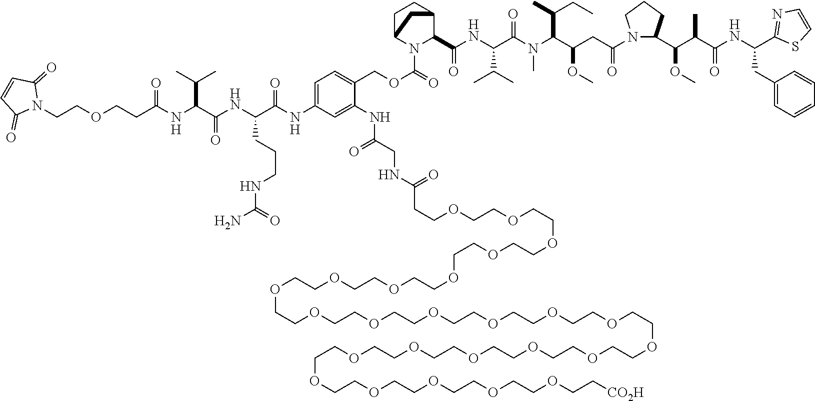 |
| L57- P1 | 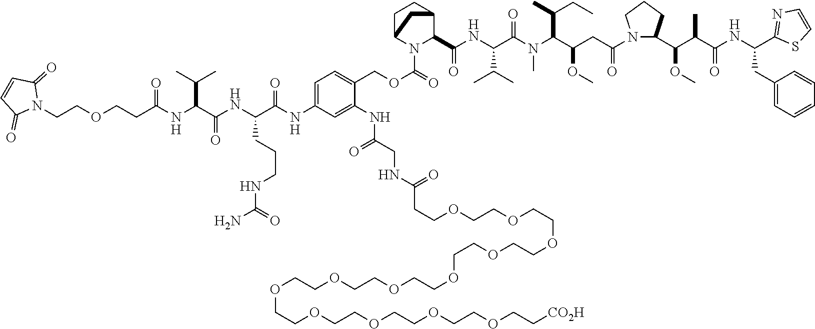 |
| L58- P1 | 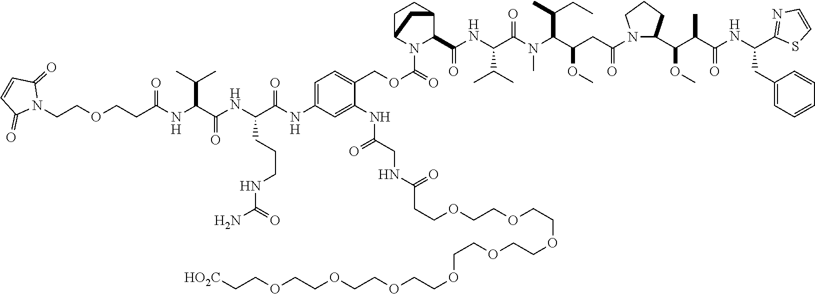 |
| L59- P1 | 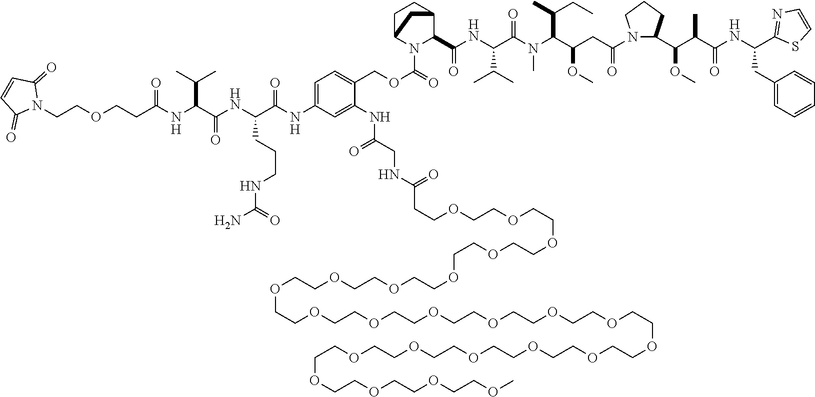 |
| L60- P1 | 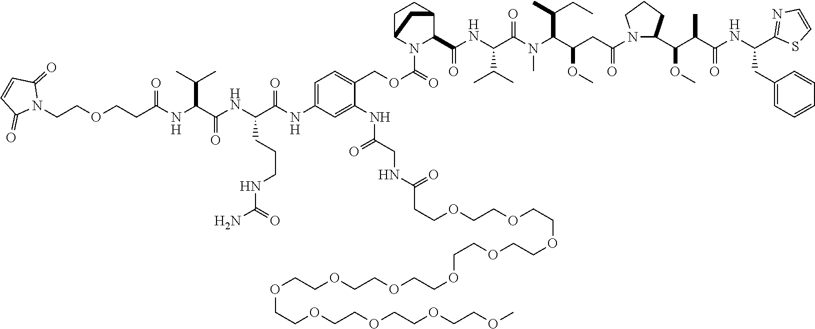 |
| L61- P1 | 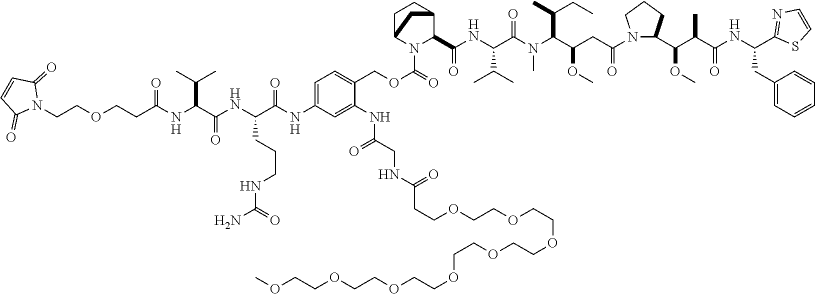 |
| L62- P1 | 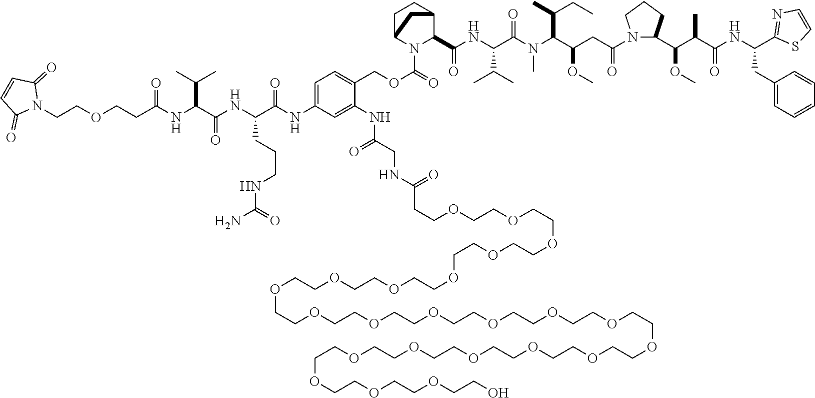 |
| L63- P1 | 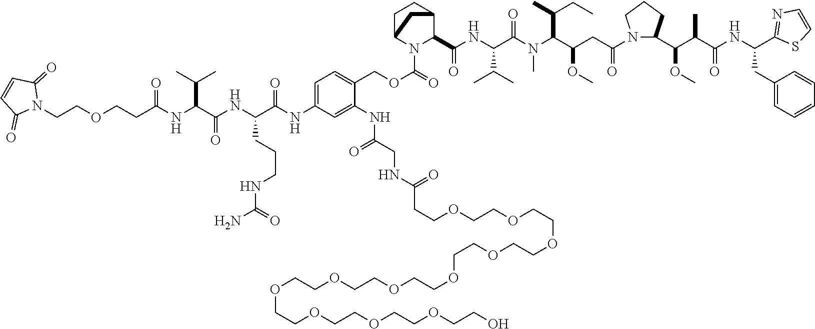 |
| L64- P1 | 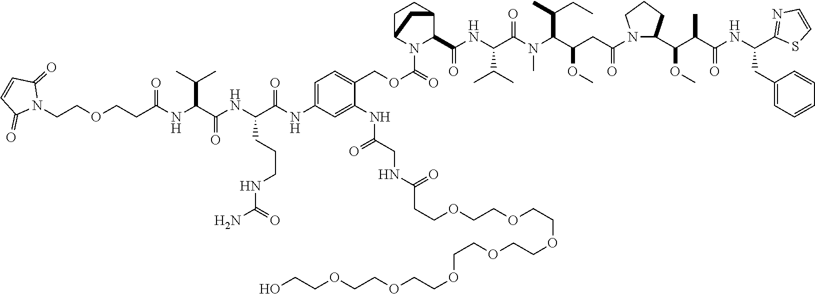 |

TABLE 4B

| Com- | |
|---|---|
| pound | |
| Num- | |
| ber | |
| L65- P1 | 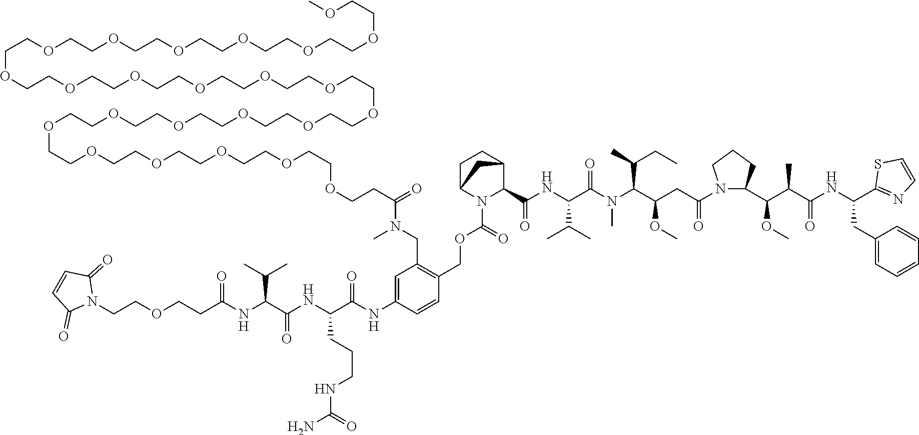 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC)C(C)C] |
| L66- P1 | 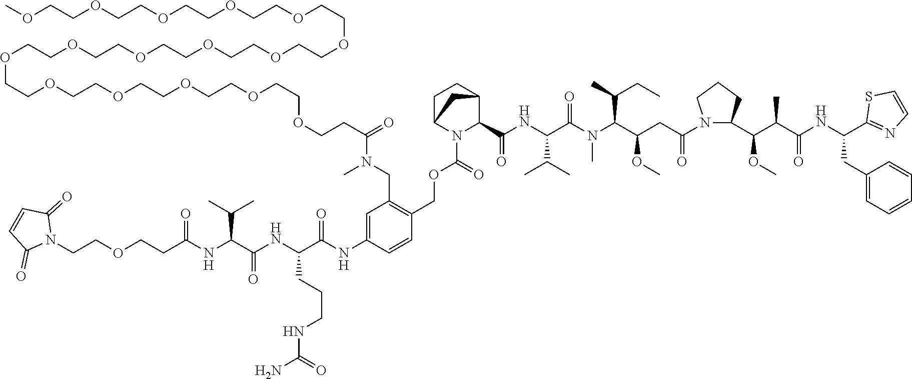 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOC)C(C)C] |
| L67- P1 | 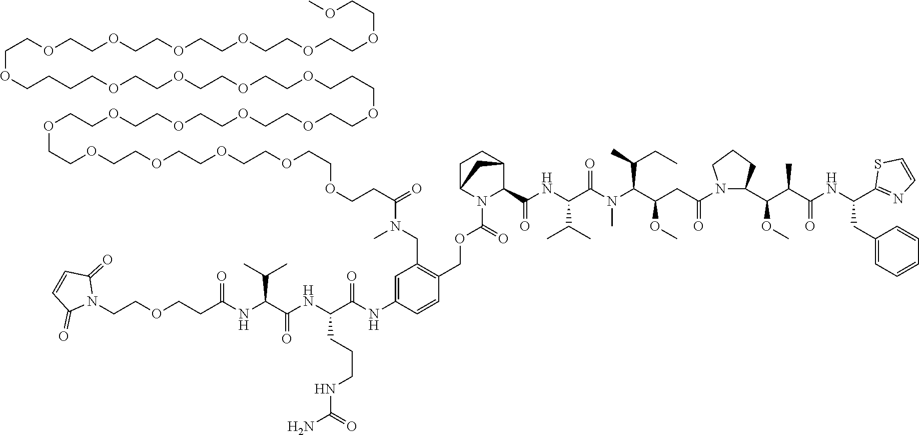 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCCOCCOCCOCCOCCCCOCCOCCOCCOCCOCCOCCOCCOC)C(C)C] |
| L68- P1 | 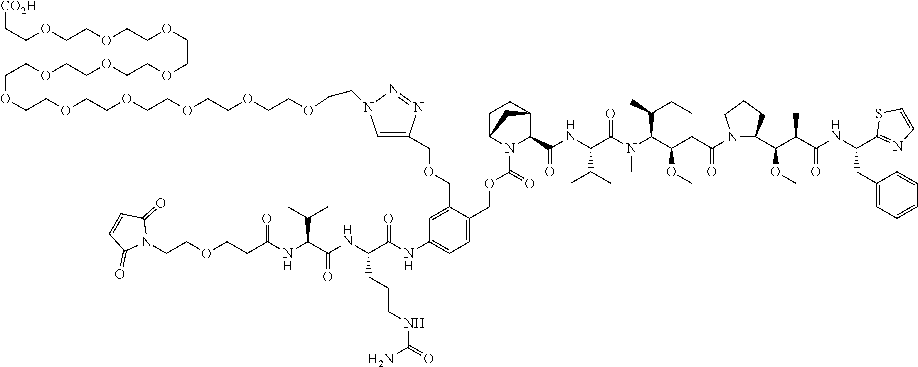 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCOCCOCCOCCOCCC(=O)O)nn1)C(C)C] |
| L69- P1 | 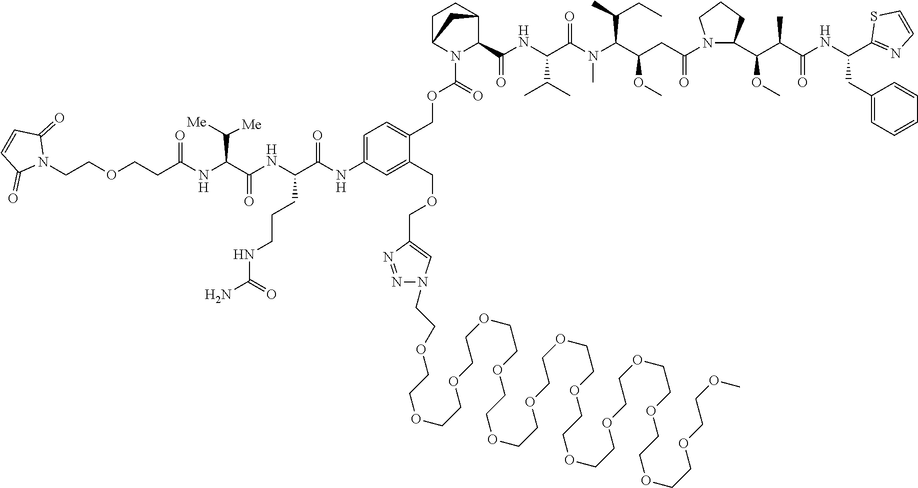 [SMILES: CC[C@H](NC(=O)CCOCCN1C(=O)C=CC1=O)C(=O)N[C@@H](CCCNC(N)=O)C(=O)Nc1ccc(COC(=O)N2[C@@H]3CC[C@@H](C3)[C@H]2C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)c2nccs2)OC)C(C)C)c(COCc2cn(CCOCCOCCOCCOCCOCCOCCOCCOCCOCCOCCOCCOCCOCCOCCOCCOC)nn2)c1 ; "Me"] |
| L70- P1 | 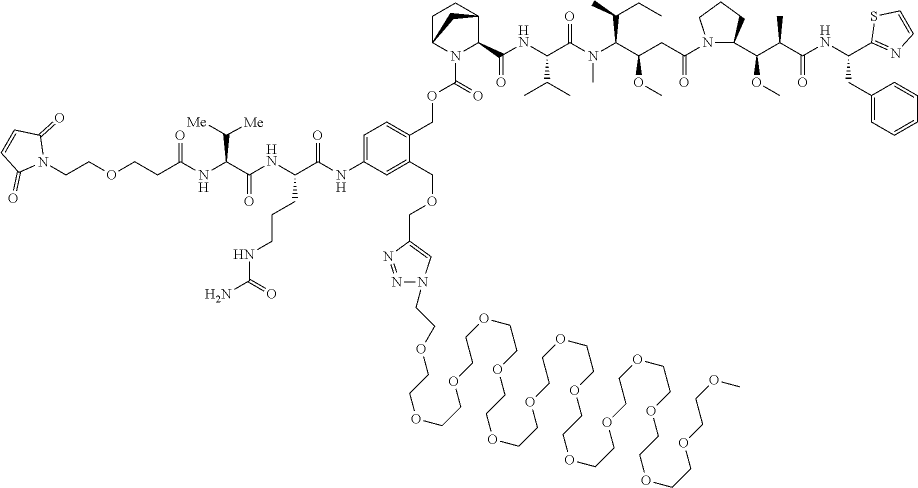 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCOCCOCCOCCOCCOCCOCCOCCOC)nn1)C(C)C] |
| L71- P1 | 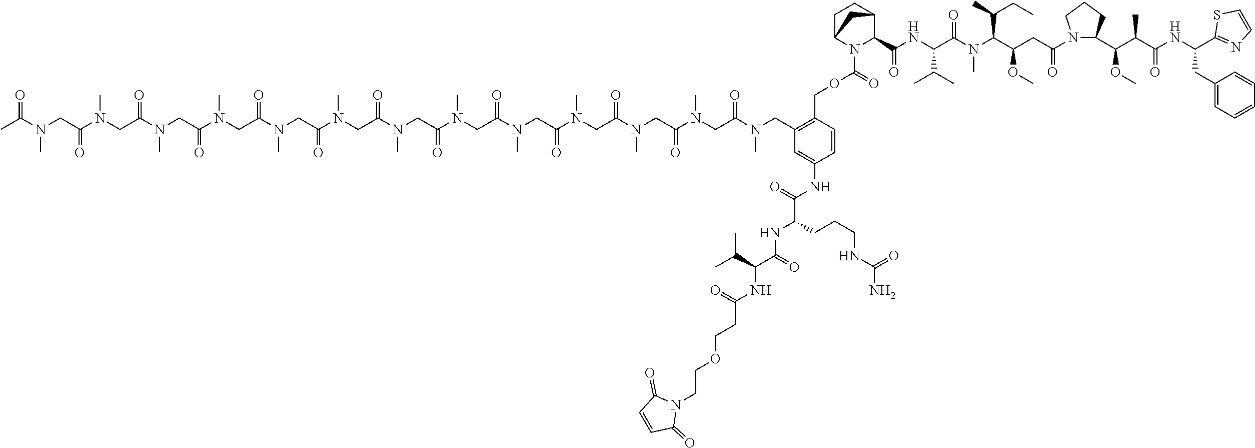 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)C(C)C] |
| L72- P1 | 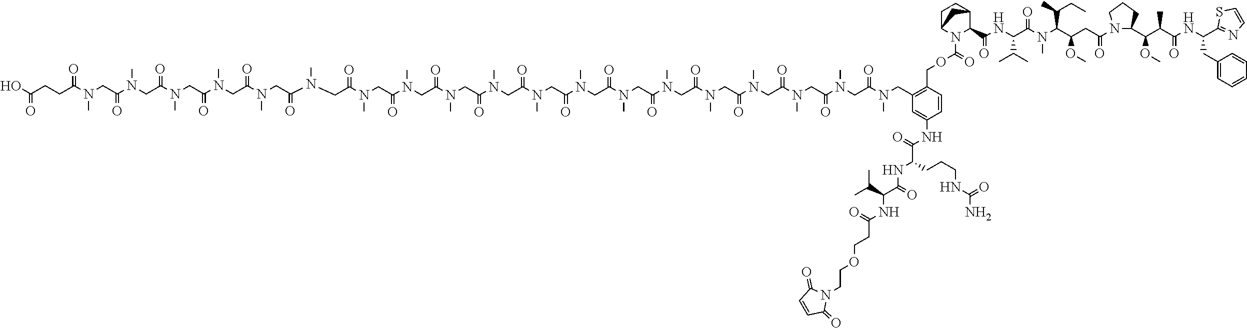 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)C(C)C] |
| L73- P1 | 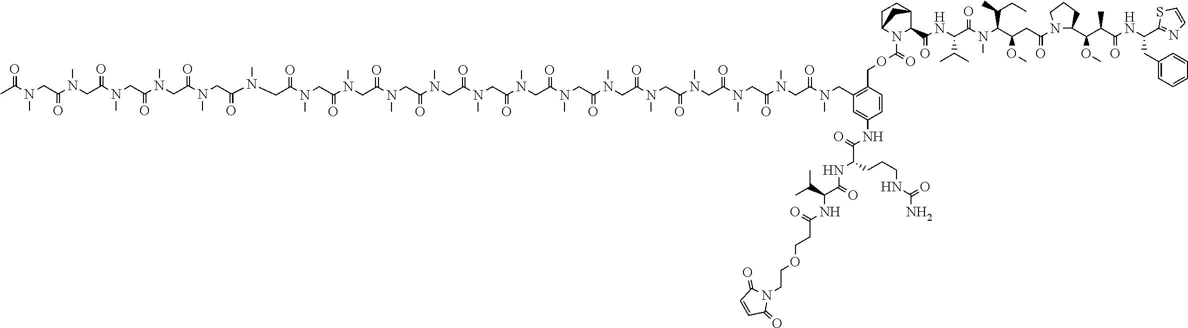 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)C(C)C] |
| L74- P1 | 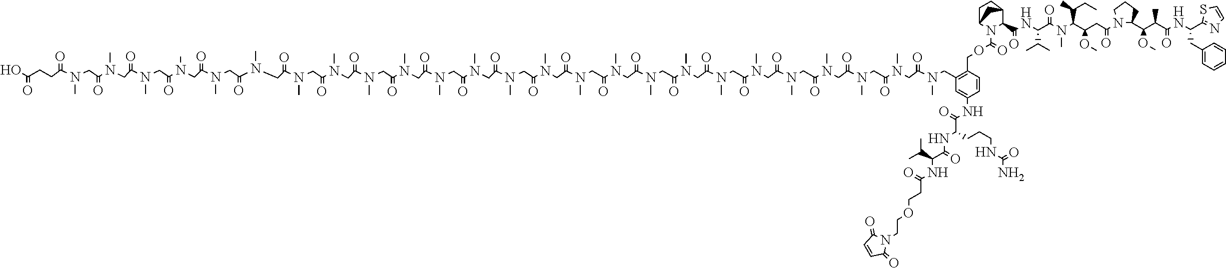 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)C(C)C] |
| L75- P1 | 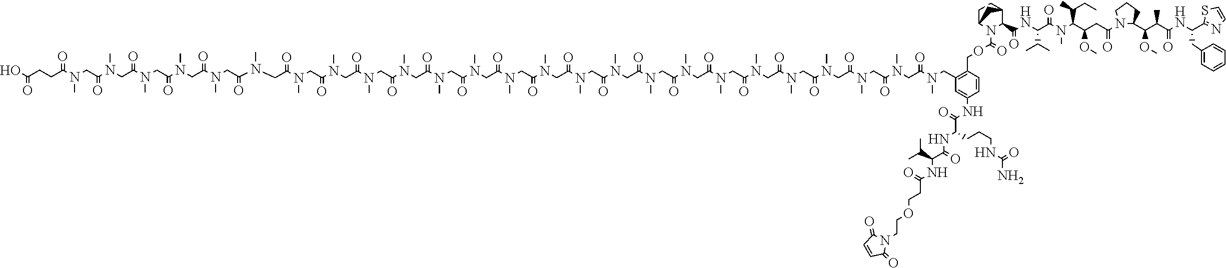 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)C(C)C] |
| L76- P1 | 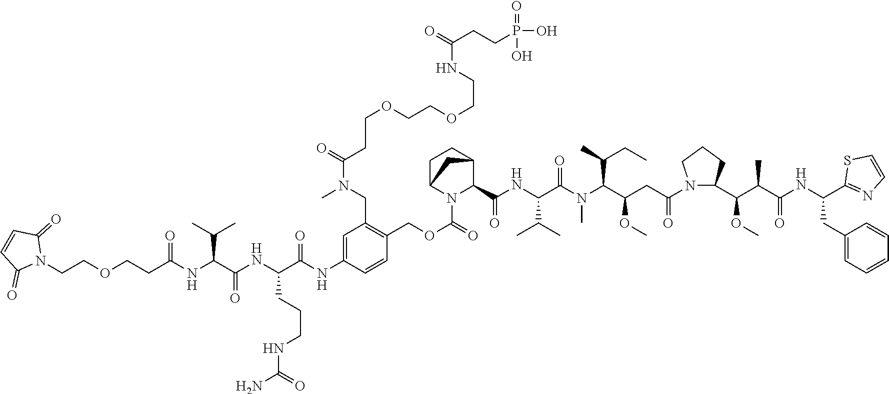 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCNC(=O)CCP(=O)(O)O)C(C)C] |
| L77- P1 | 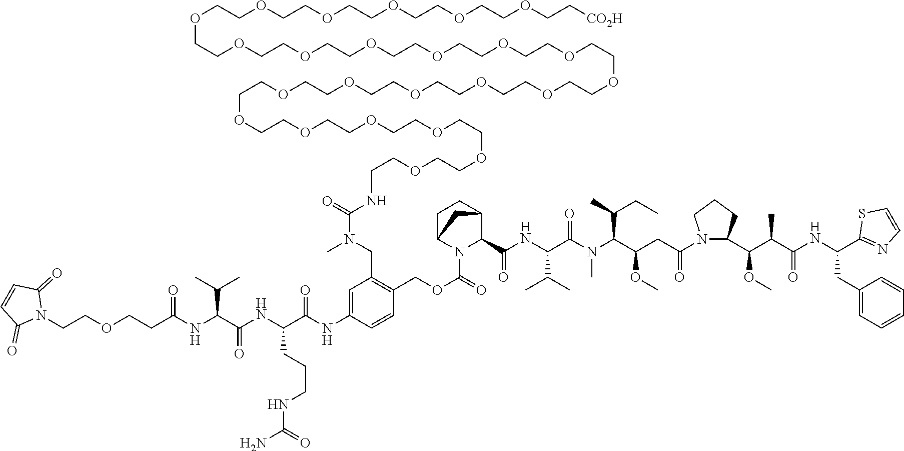 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)C(C)C] |
| L78- P1 | 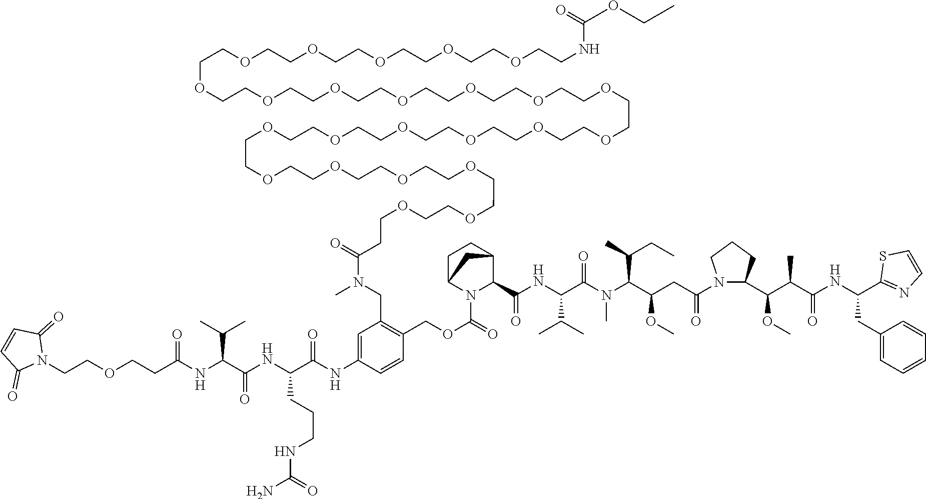 [SMILES: CCOC(=O)NCCOCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N1[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C] |
| L79- P1 | 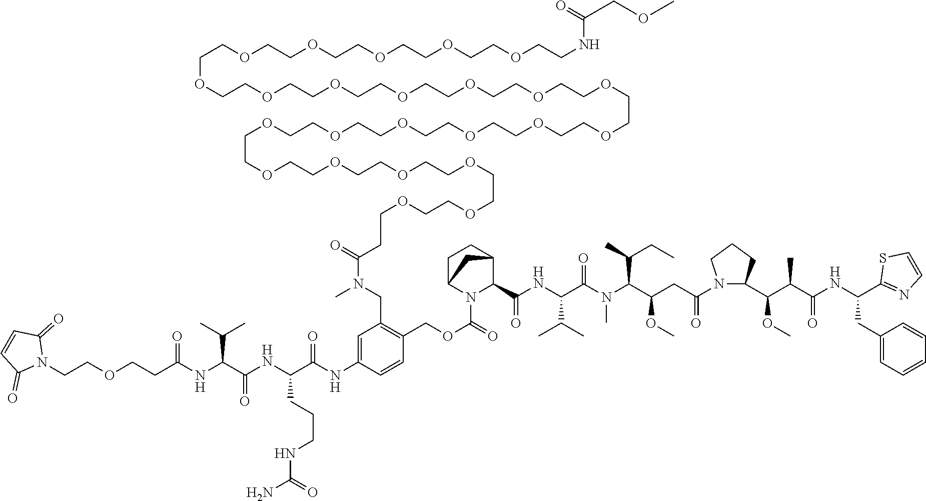 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCNC(=O)COC)C(C)C] |
| L80- P1 | 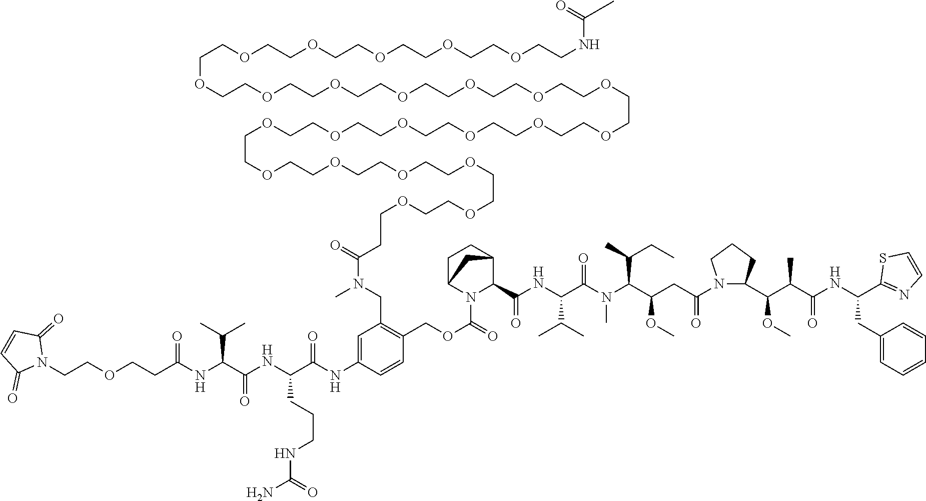 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCNC(C)=O)C(C)C] |
| L81- P1 | 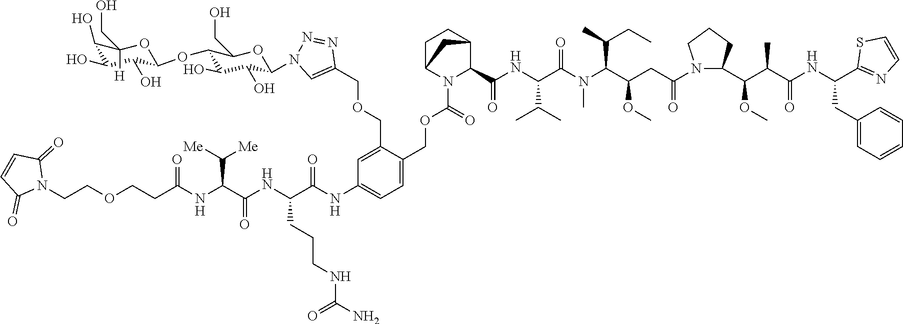 |
| L82- P1 | 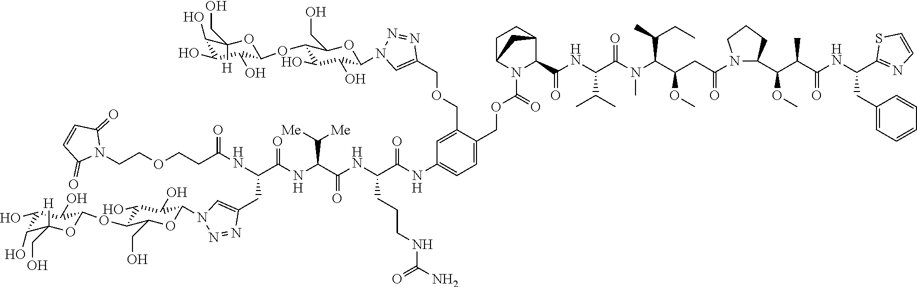 |
| L83- P1 | 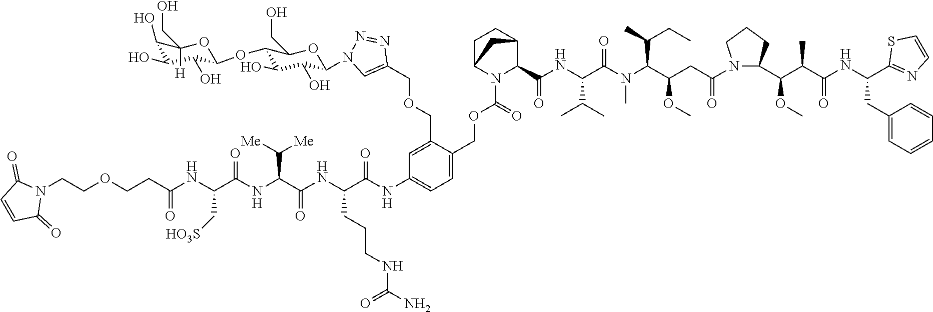 |
| L84- P1 | 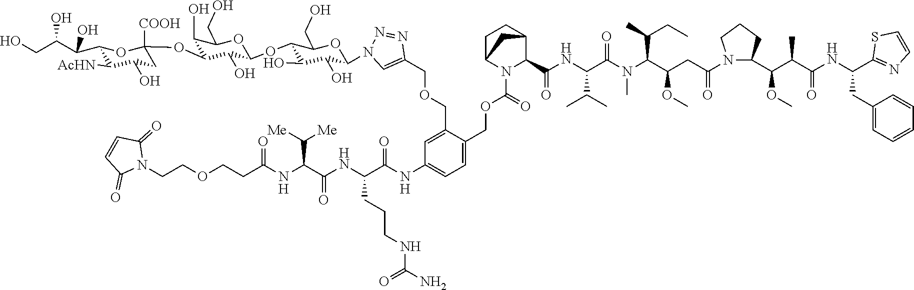 |
| L85- P1 | 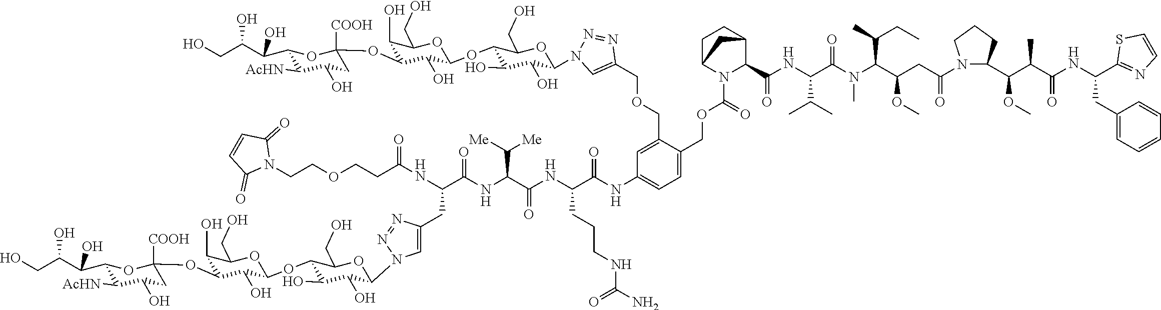 |
| L86- P1 | 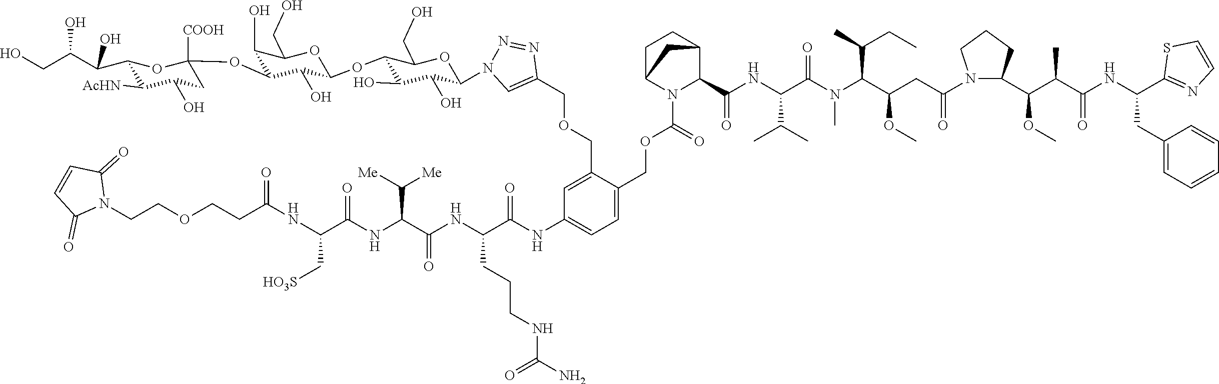 |
| L87- P1 | 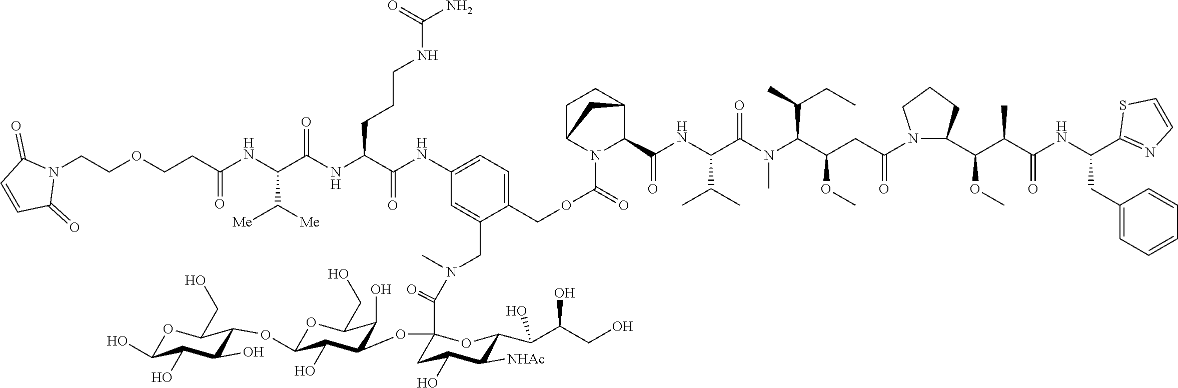 |
| L88- P1 | 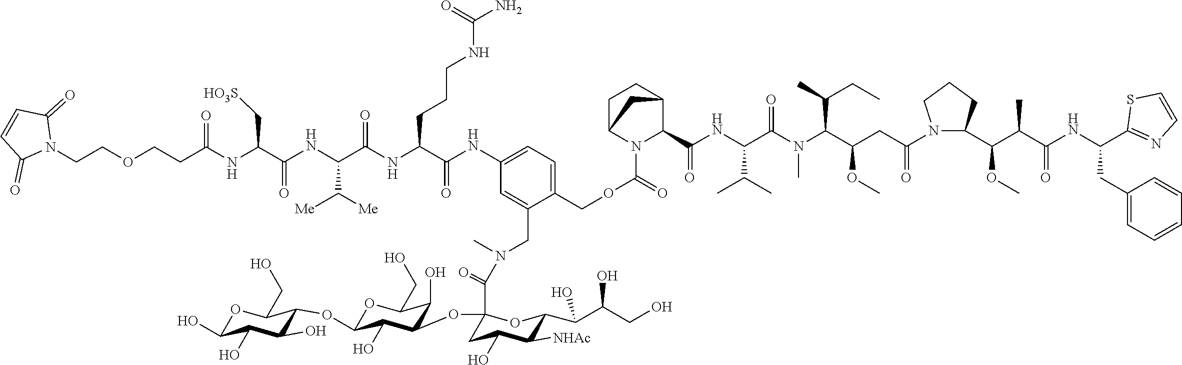 |
| L89- P1 | 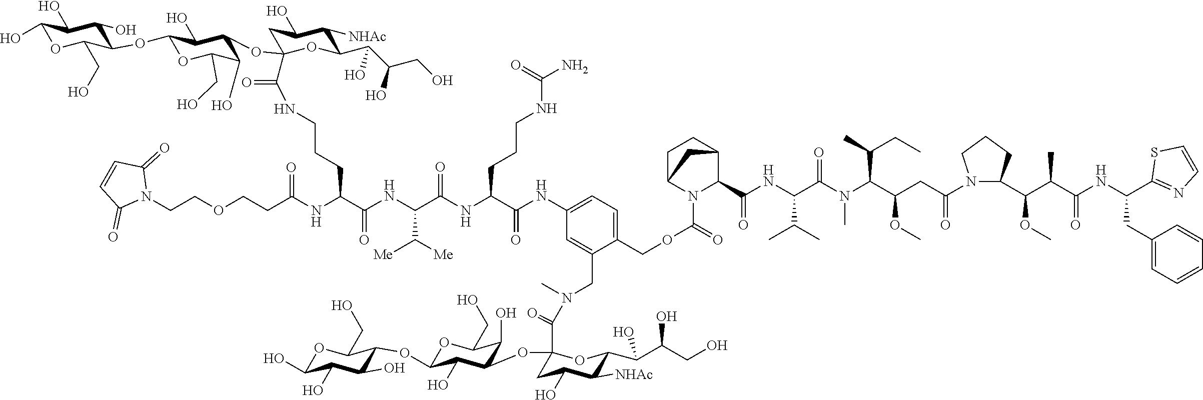 |
| L90- P1 | 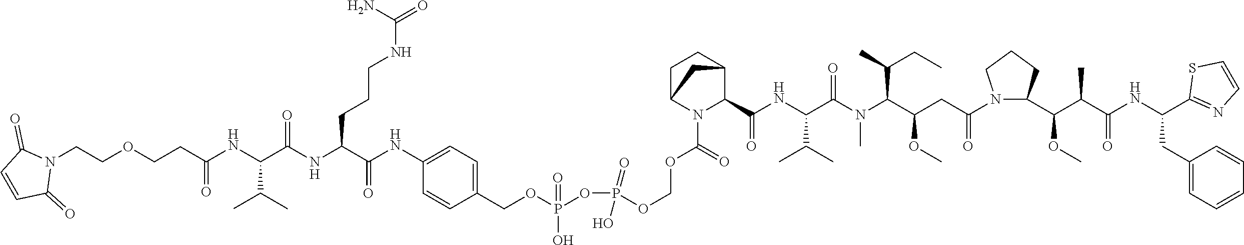 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCOP(=O)(O)OP(=O)(O)OCc1ccc(NC(=O)[C@H](CCCNC(N)=O)NC(=O)[C@@H](NC(=O)CCOCCN2C(=O)C=CC2=O)C(C)C)cc1)C(C)C] |
| L91- P1 | 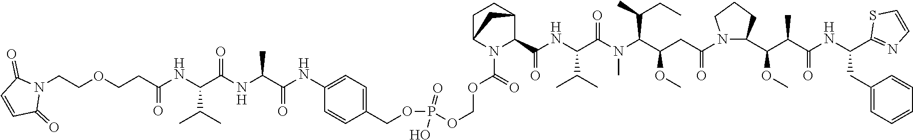 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCOP(=O)(O)OCc1ccc(NC(=O)[C@H](C)NC(=O)[C@@H](NC(=O)CCOCCN2C(=O)C=CC2=O)C(C)C)cc1)C(C)C] |
| L92- P1 | 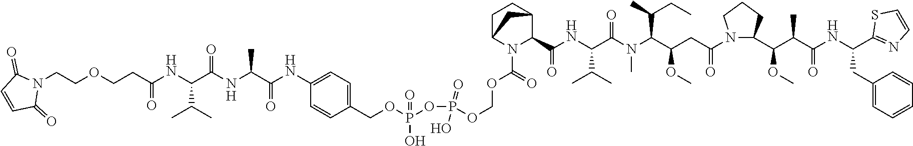 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCOP(=O)(O)OP(=O)(O)OCc1ccc(NC(=O)[C@H](C)NC(=O)[C@@H](NC(=O)CCOCCN2C(=O)C=CC2=O)C(C)C)cc1)C(C)C] |
| L93- P1 | 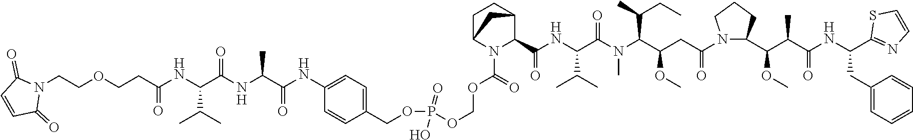 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCOP(=O)(O)OCc1ccc(NC(=O)[C@H](C)NC(=O)[C@@H](NC(=O)CCOCCN2C(=O)C=CC2=O)C(C)C)cc1)C(C)C] |
| L94- P1 | 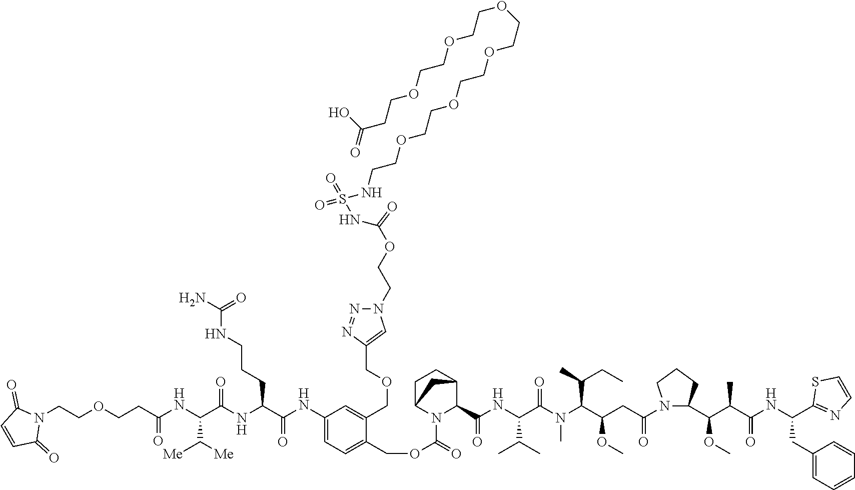 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCOCCOCCOCCOCCOCCC(=O)O)nn1)C(C)C] |
| L95- P1 | 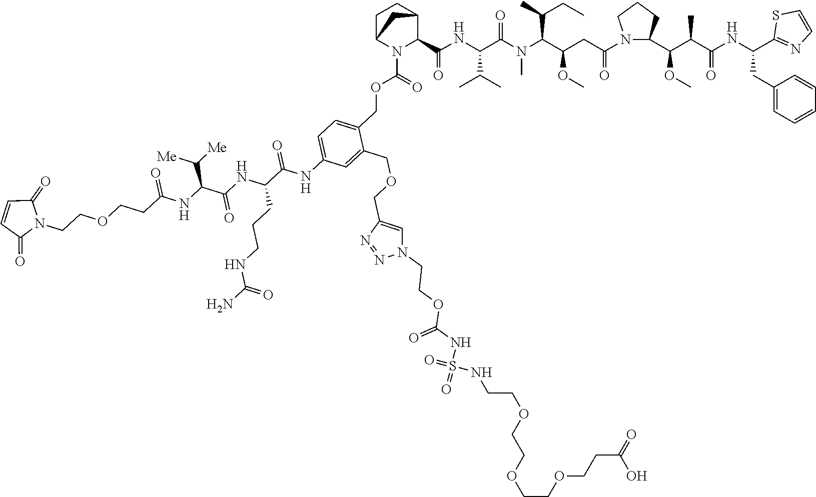 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCOCCOCCC(=O)O)nn1)C(C)C] |
| L96- P1 | 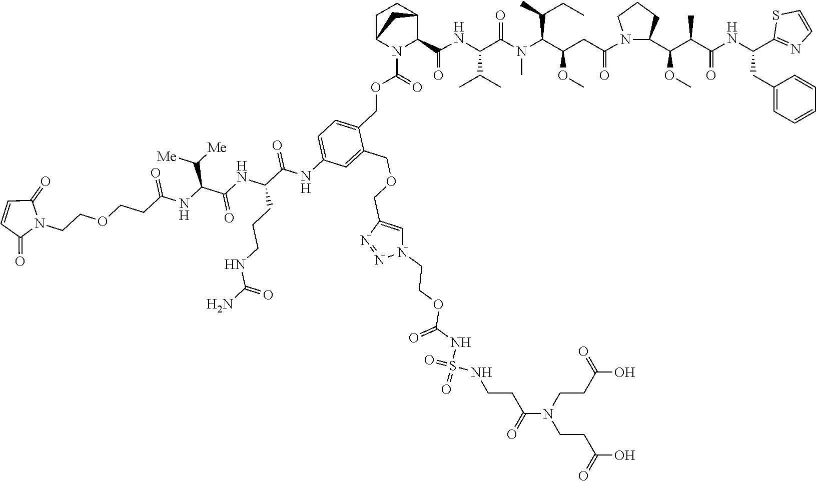 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCC(=O)N(CCC(=O)O)CCC(=O)O)nn1)C(C)C] |
| L97- P1 | 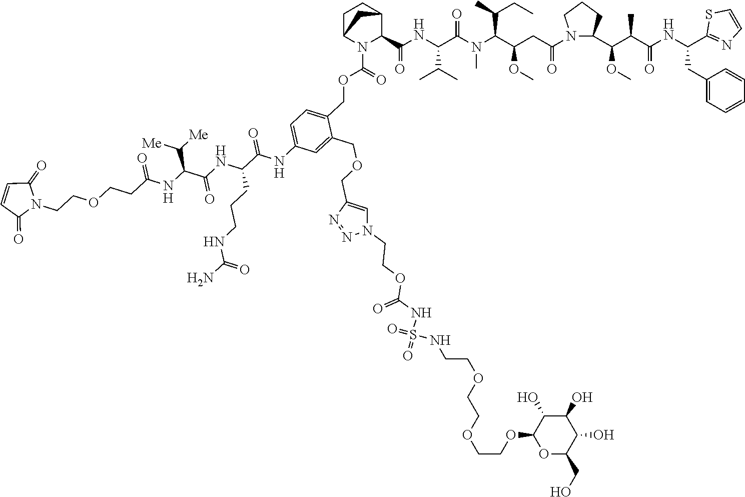 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCOCCO[C@@H]2O[C@H](CO)[C@@H](O)[C@H](O)[C@H]2O)nn1)C(C)C] |
| L98- P1 | 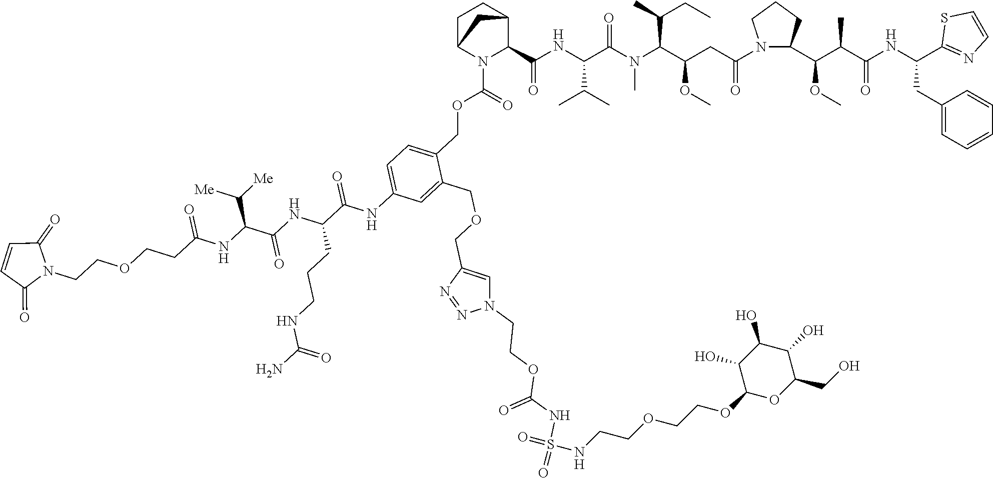 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCO[C@@H]2O[C@H](CO)[C@@H](O)[C@H](O)[C@H]2O)nn1)C(C)C] |
| L99- P1 | 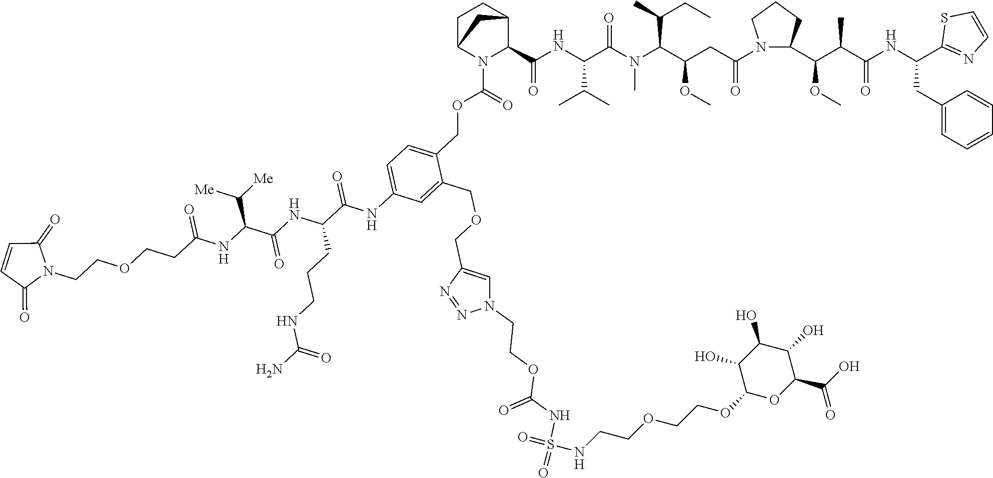 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCO[C@H]2O[C@H](C(=O)O)[C@@H](O)[C@H](O)[C@H]2O)nn1)C(C)C] |
| L100- P1 | 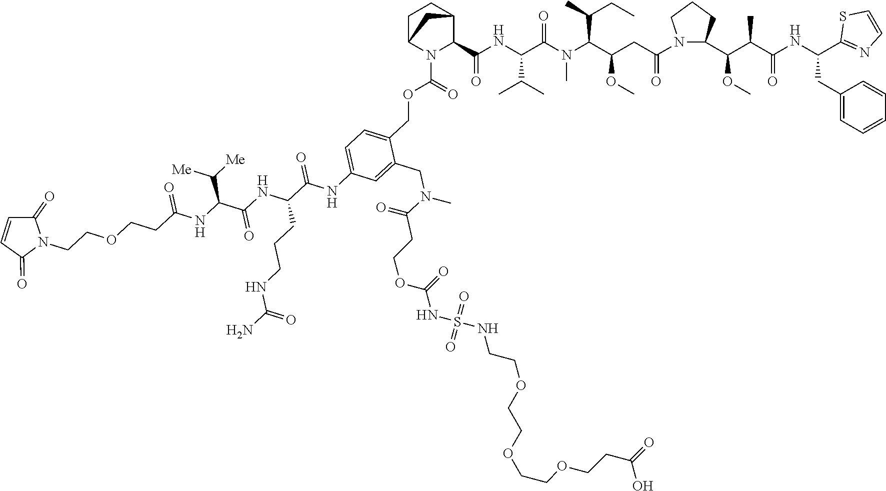 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOC(=O)NS(=O)(=O)NCCOCCOCCOCCC(=O)O)C(C)C] |
| L101- P1 | 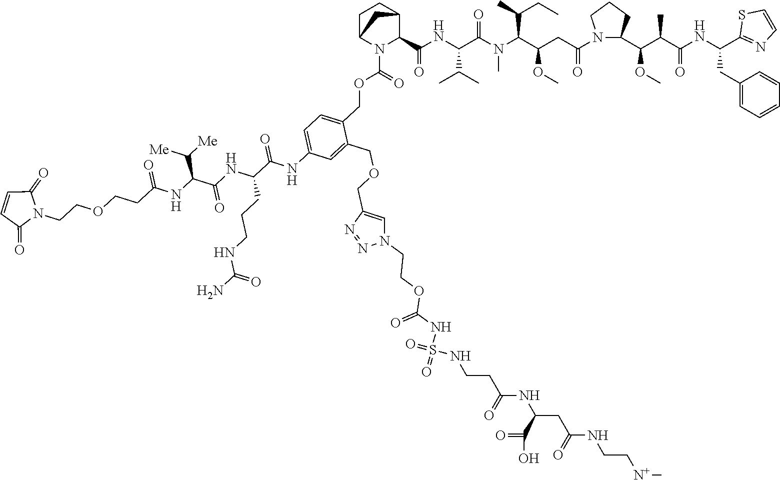 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCC(=O)N[C@@H](CC(=O)NCC[NH2+]C)C(=O)O)nn1)C(C)C] |
| L102- P1 | 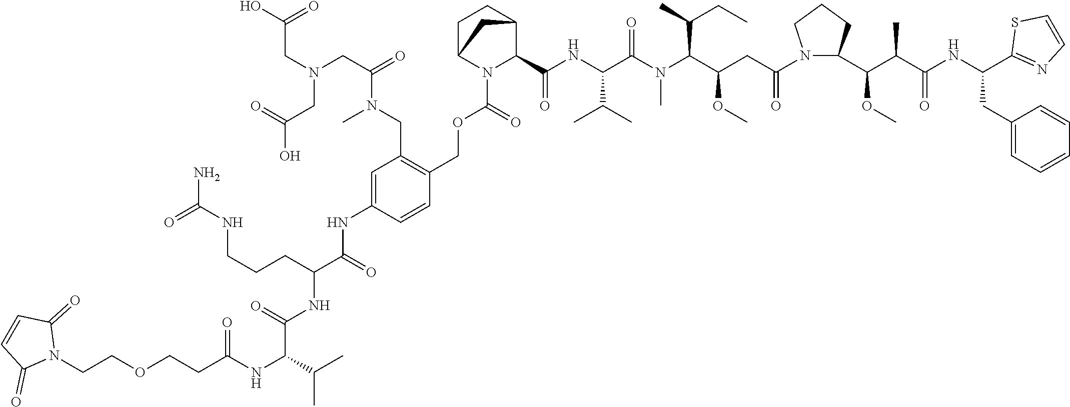 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)C(CCCNC(N)=O)NC(=O)[C@@H](NC(=O)CCOCCN2C(=O)C=CC2=O)C(C)C)cc1CN(C)C(=O)CN(CC(=O)O)CC(=O)O)C(C)C] |
| L103- P1 | 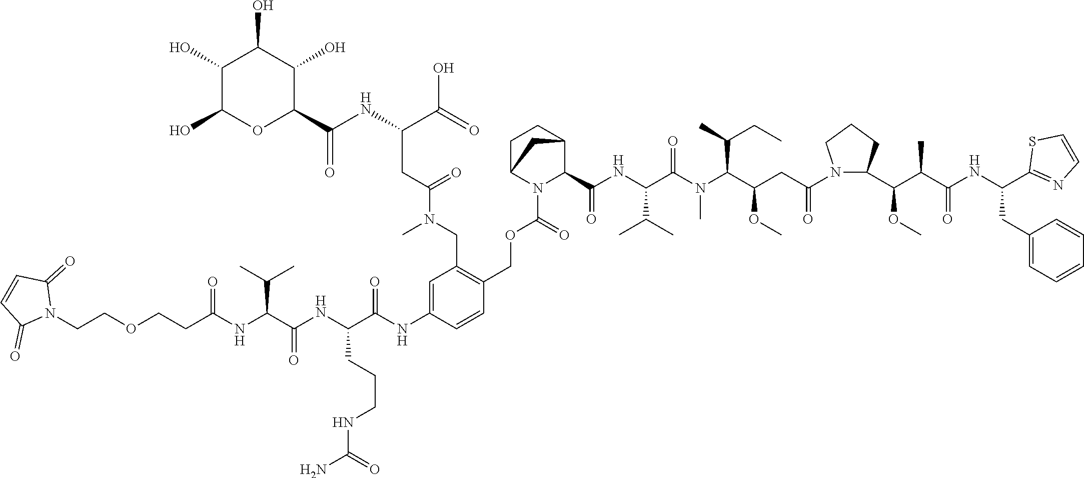 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)[C@H]1O[C@@H](O)[C@H](O)[C@@H](O)[C@@H]1O)C(=O)O)C(C)C] |
| L104- P1 | 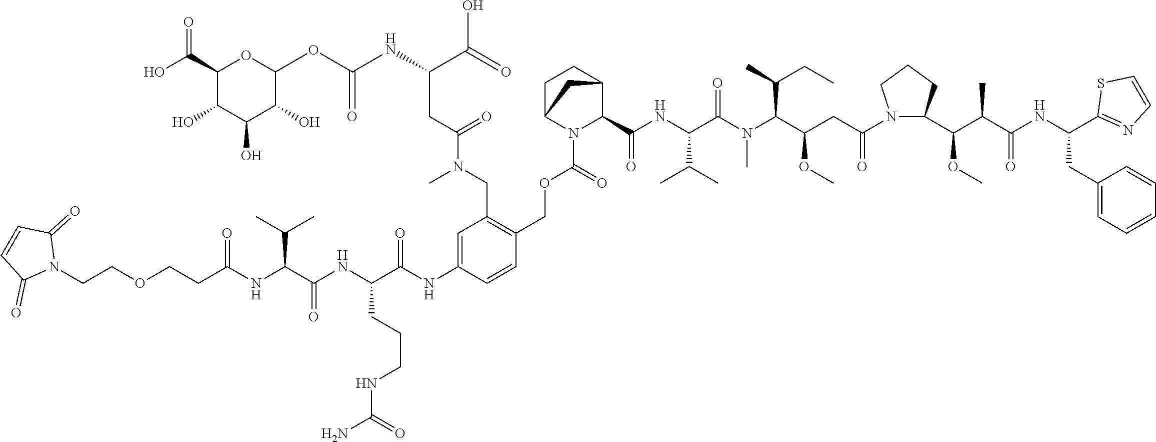 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)OC1O[C@H](C(=O)O)[C@@H](O)[C@H](O)[C@H]1O)C(=O)O)C(C)C] |
| L105- P1 | 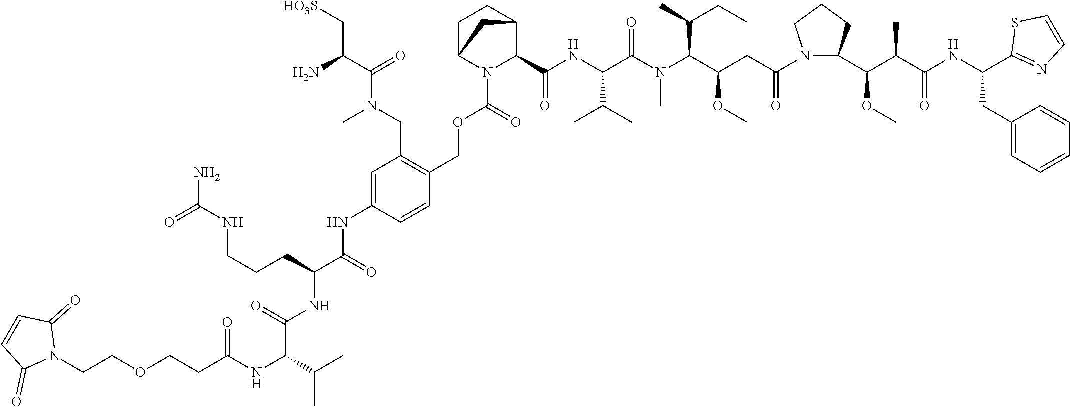 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](N)CS(=O)(=O)O)C(C)C] |
| L106- P1 | 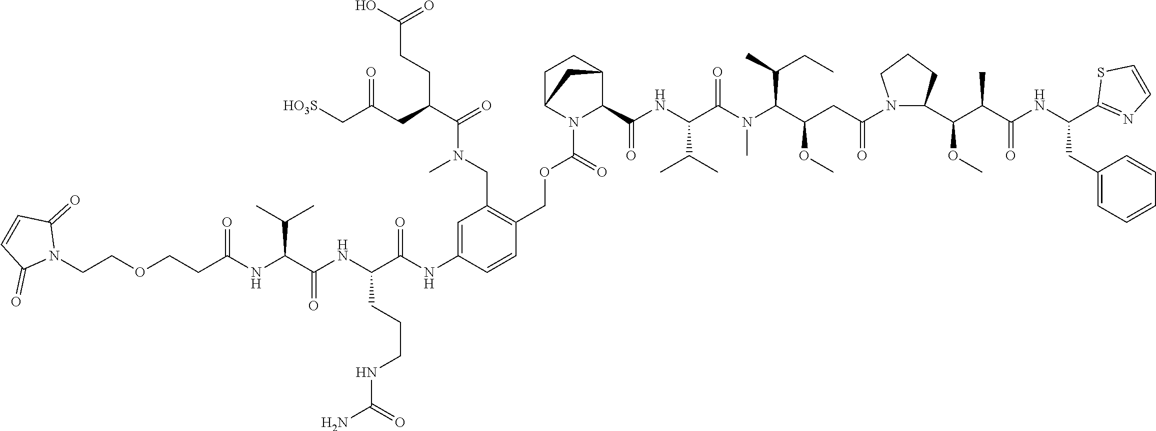 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCC(=O)O)CC(=O)CS(=O)(=O)O)C(C)C] |
| L107- P1 | 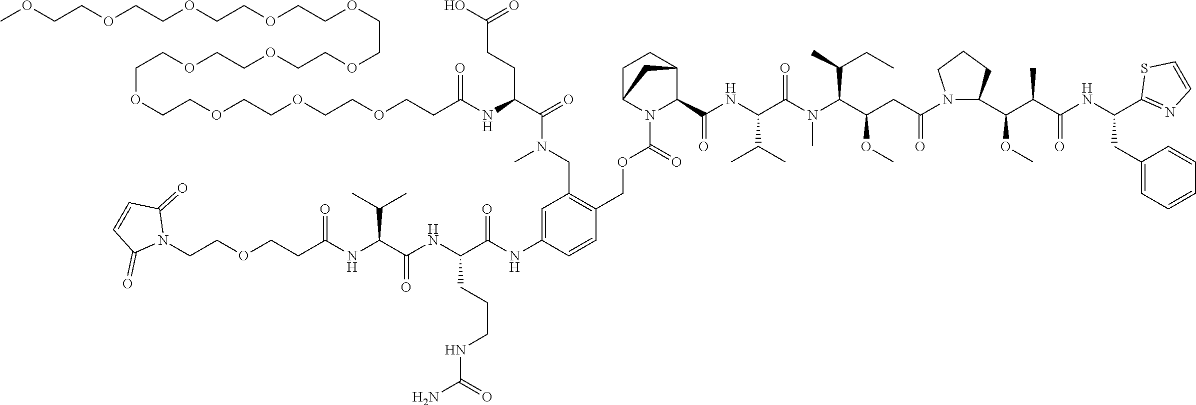 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCC(=O)O)NC(=O)CCOCCOCCOCCOCCOCCOCCOCCOCCOCCOCCOCCOC)C(C)C] |
| L108- P1 | 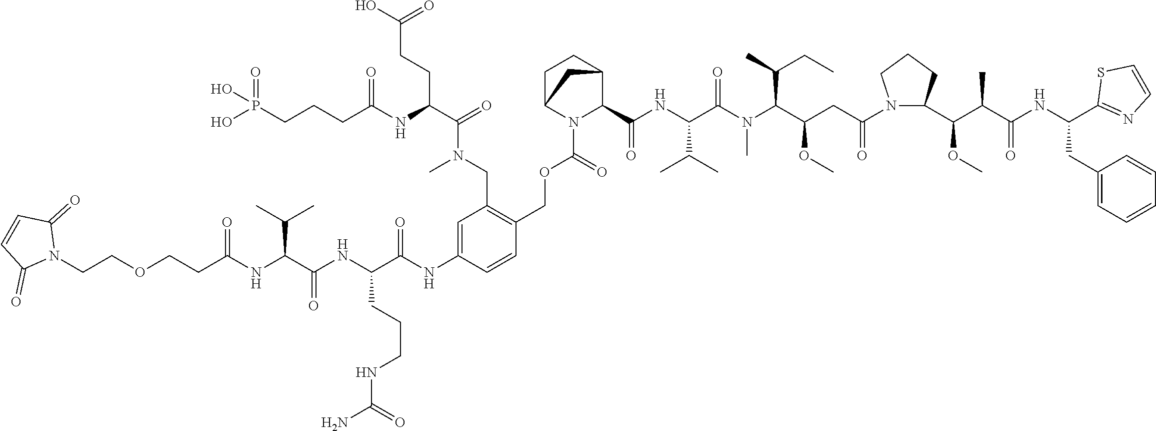 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCC(=O)O)NC(=O)CCCP(=O)(O)O)C(C)C] |
| L109- P1 | 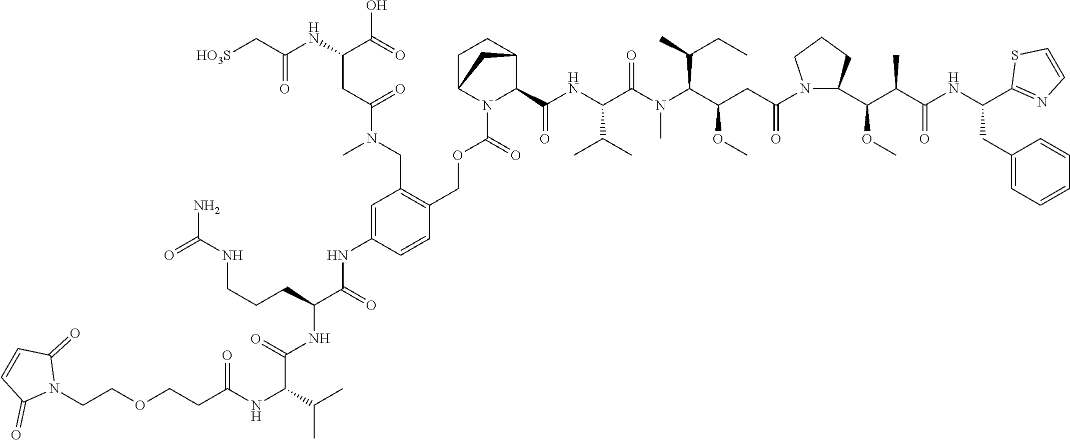 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CS(=O)(=O)O)C(=O)O)C(C)C] |
| L110- P1 | 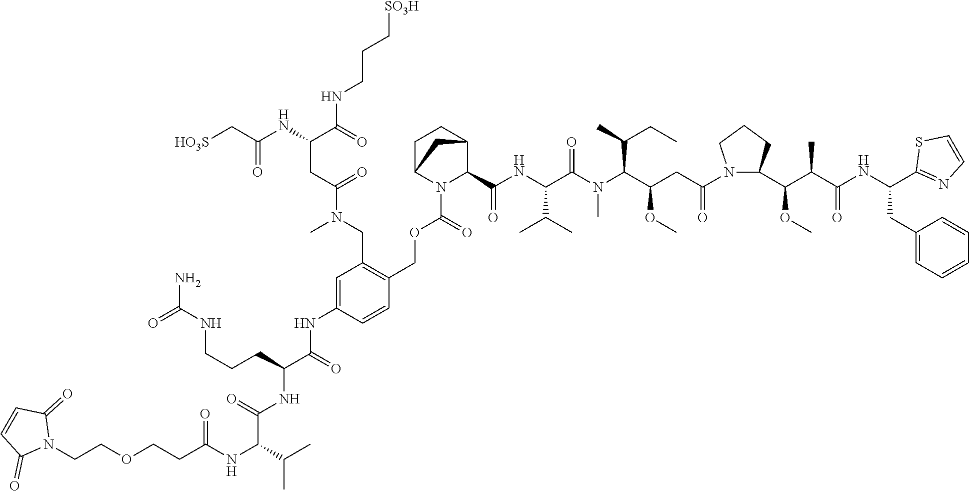 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CS(=O)(=O)O)C(=O)NCCCS(=O)(=O)O)C(C)C] |
| L111- P1 | 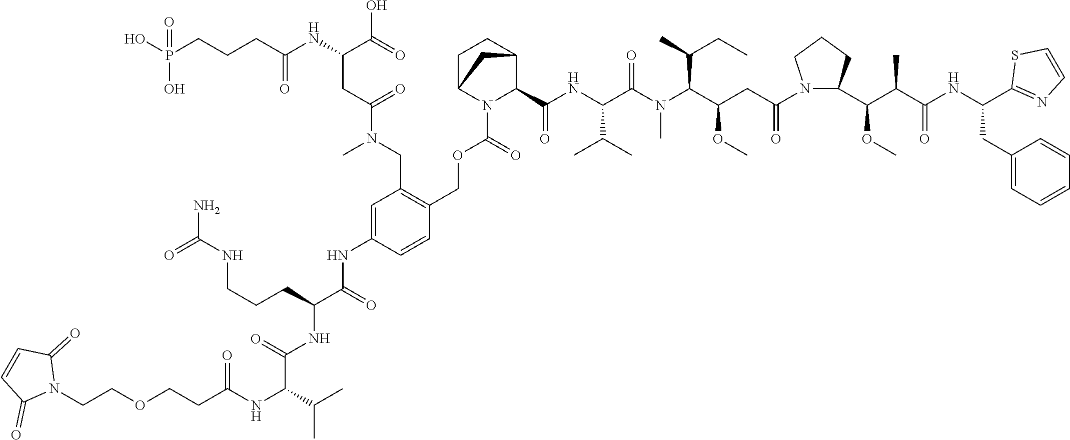 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CCCP(=O)(O)O)C(=O)O)C(C)C] |
| L112- P1 | 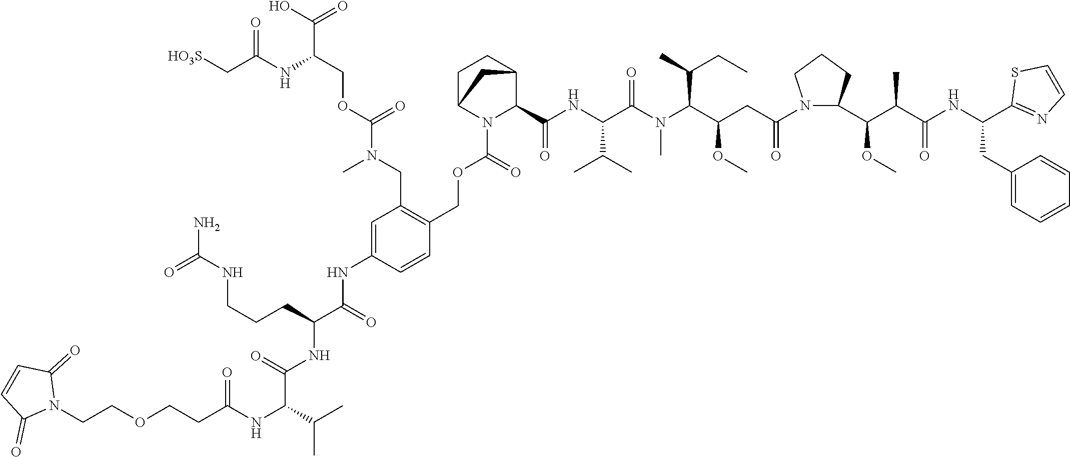 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@H](NC(=O)CS(=O)(=O)O)C(=O)O)C(C)C] |
| L113- P1 | 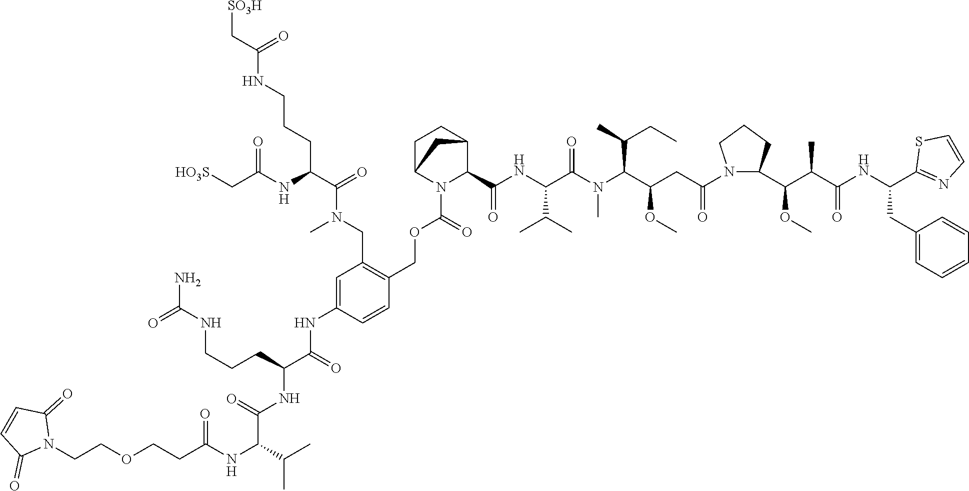 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCCNC(=O)CS(=O)(=O)O)NC(=O)CS(=O)(=O)O)C(C)C] |
| L114- P1 | 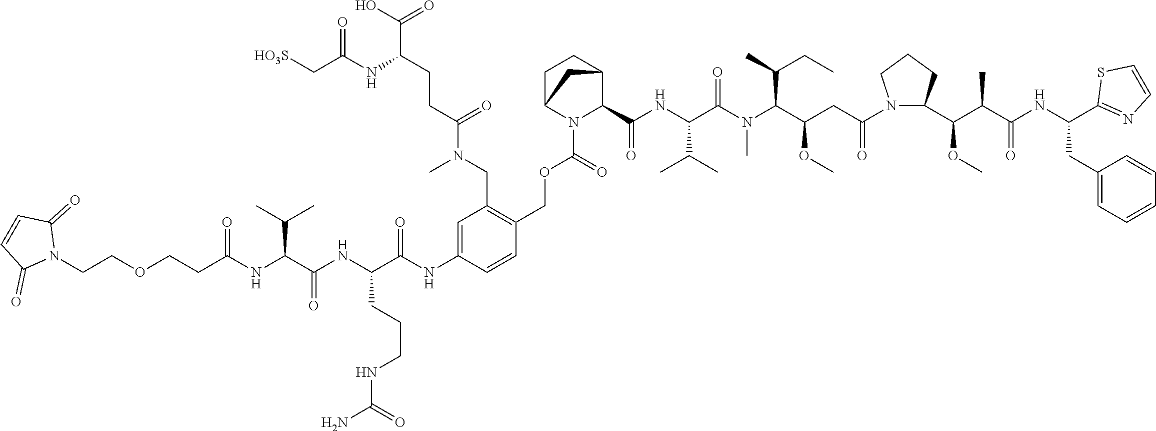 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CC[C@H](NC(=O)CS(=O)(=O)O)C(=O)O)C(C)C] |
| L115- P1 | 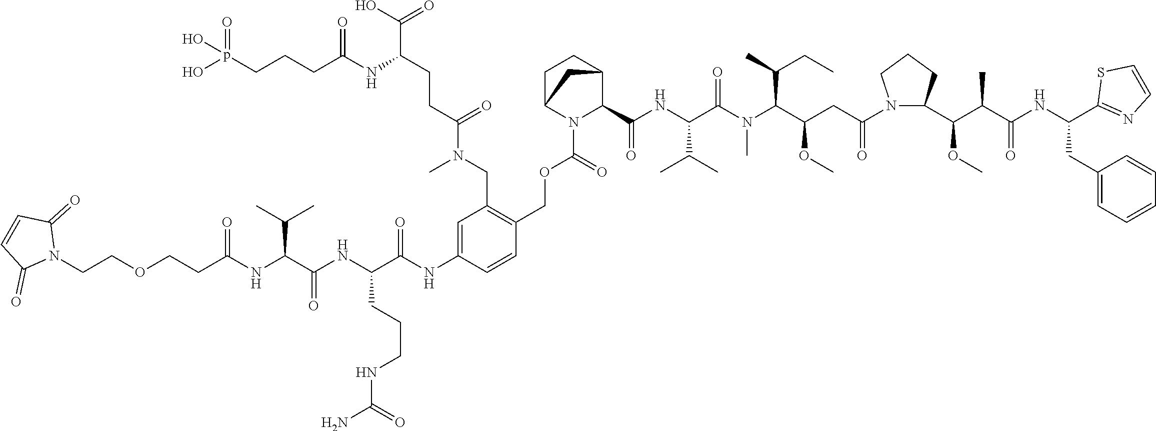 |
| L116- P1 | 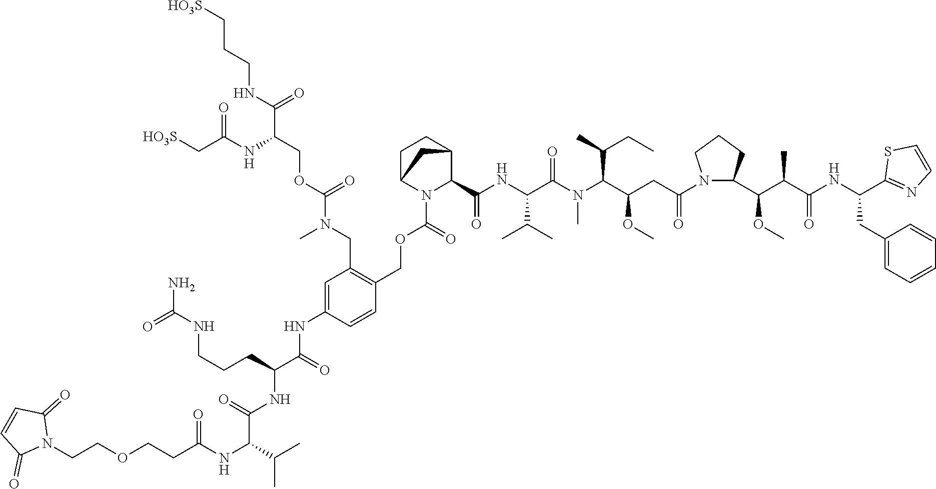 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@H](NC(=O)CS(=O)(=O)O)C(=O)NCCCS(=O)(=O)O)C(C)C] |
| L117- P1 | 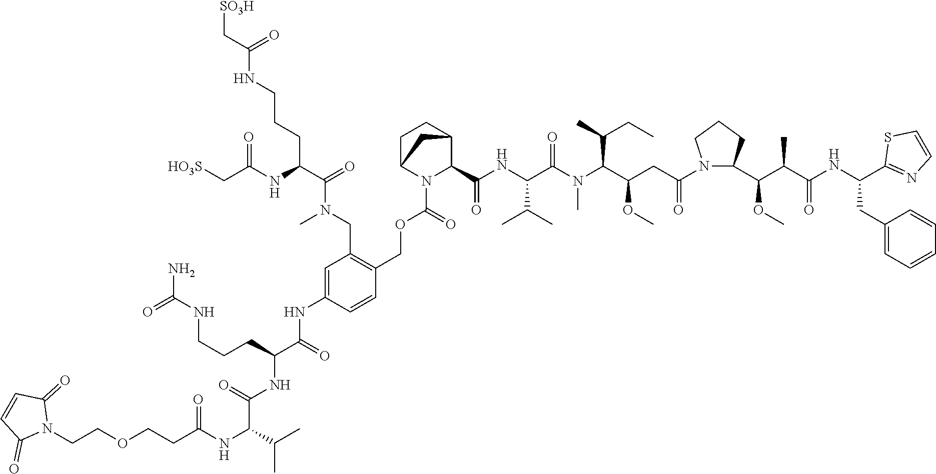 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCCNC(=O)CS(=O)(=O)O)NC(=O)CS(=O)(=O)O)C(C)C] |
| L118- P1 | 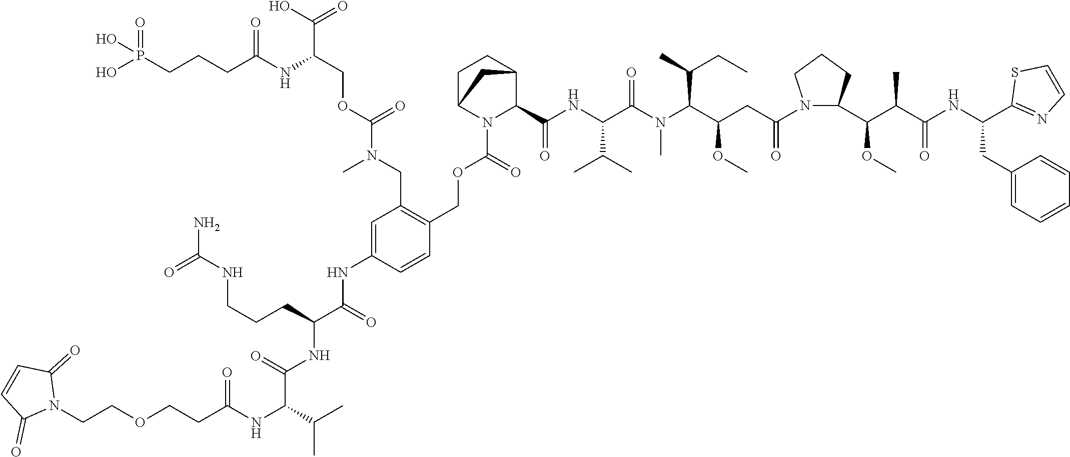 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@H](NC(=O)CCCP(=O)(O)O)C(=O)O)C(C)C] |
| L119- P1 | 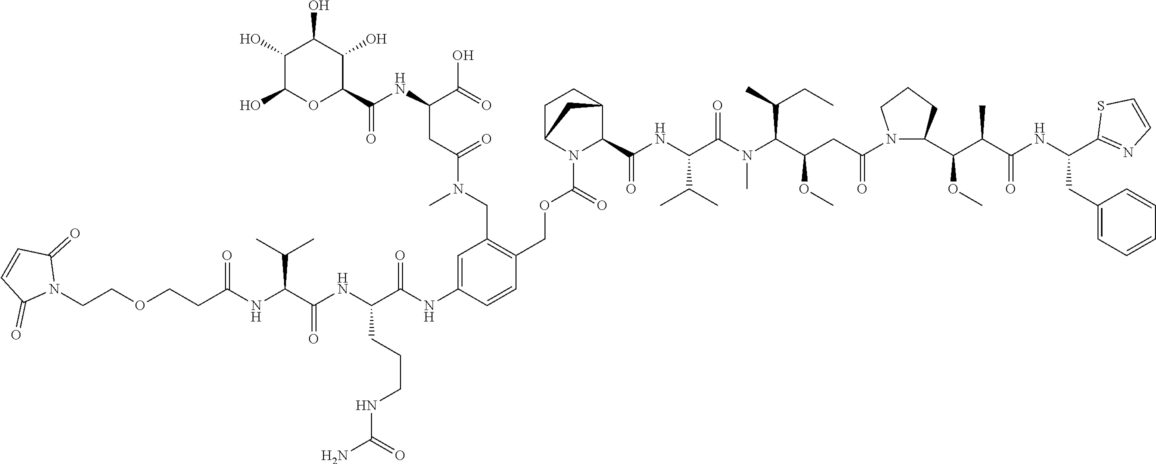 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@@H](NC(=O)[C@H]1O[C@@H](O)[C@H](O)[C@@H](O)[C@@H]1O)C(=O)O)C(C)C] |
| L120- P1 | 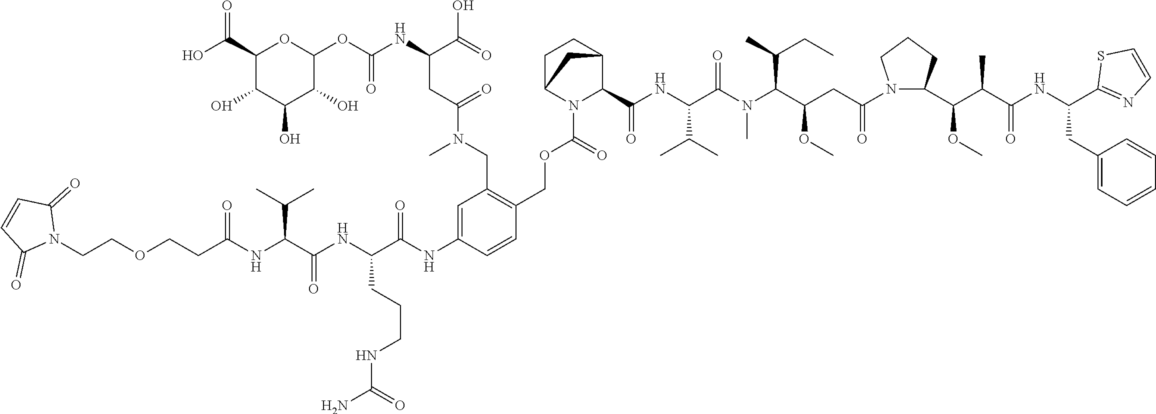 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@@H](NC(=O)OC1O[C@H](C(=O)O)[C@@H](O)[C@H](O)[C@H]1O)C(=O)O)C(C)C] |
| L121- P1 | 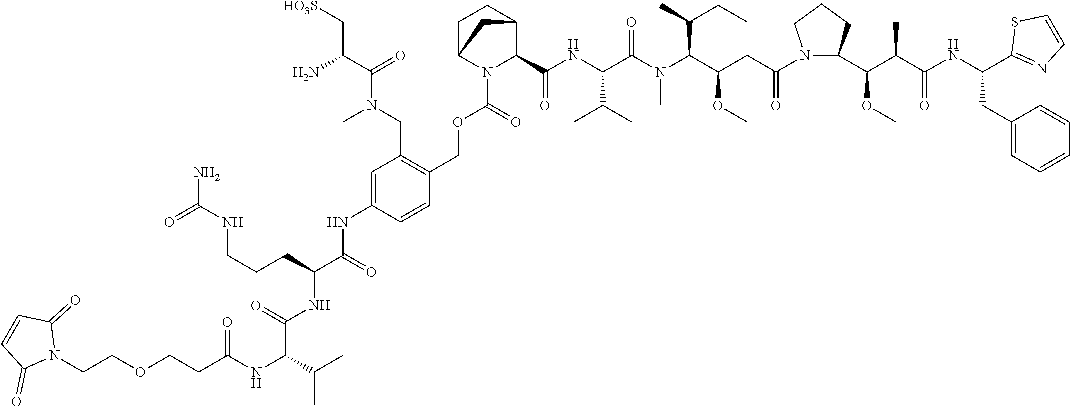 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](N)CS(=O)(=O)O)C(C)C] |
| L122- P1 | 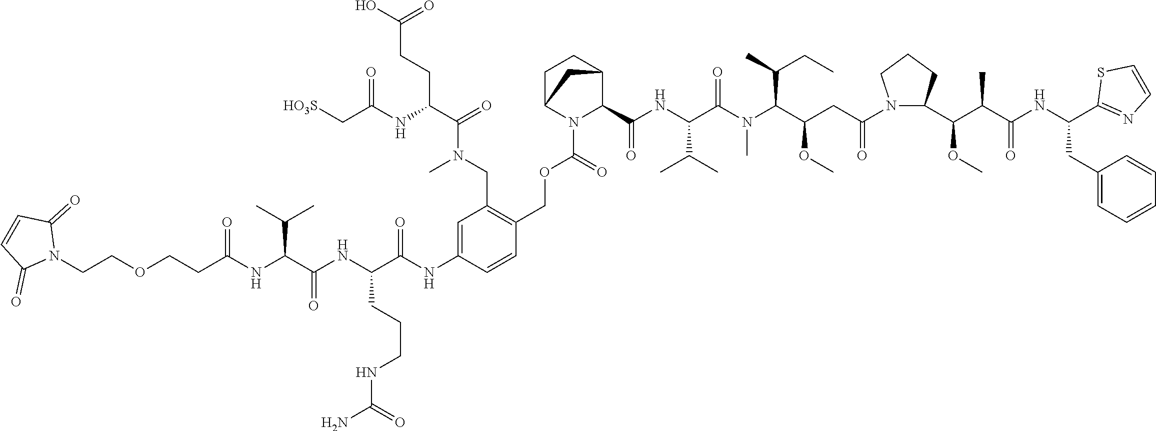 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](CCC(=O)O)NC(=O)CS(=O)(=O)O)C(C)C] |
| L123- P1 | 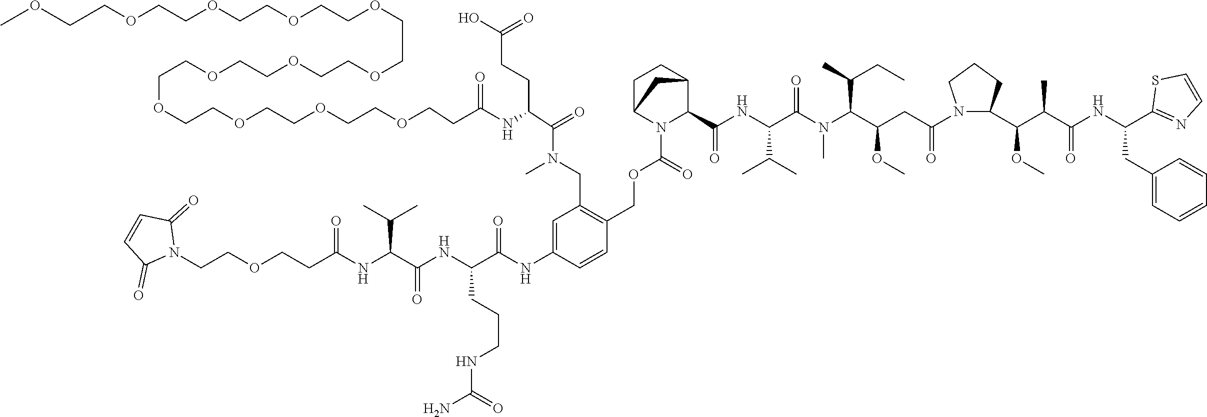 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](CCC(=O)O)NC(=O)CCOCCOCCOCCOCCOCCOCCOCCOCCOCCOCCOCCOC)C(C)C] |
| L124- P1 | 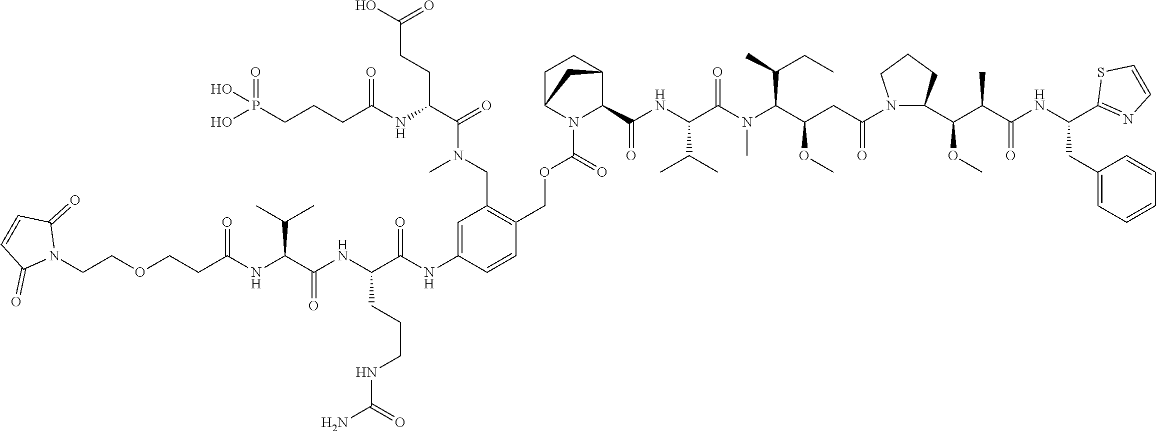 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](CCC(=O)O)NC(=O)CCCP(=O)(O)O)C(C)C] |
| L125- P1 | 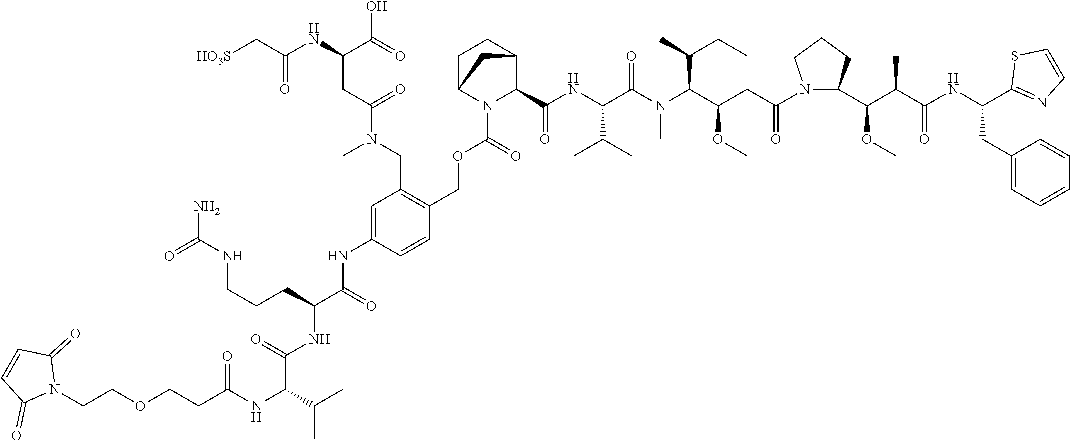 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@@H](NC(=O)CS(=O)(=O)O)C(=O)O)C(C)C] |
| L126- P1 | 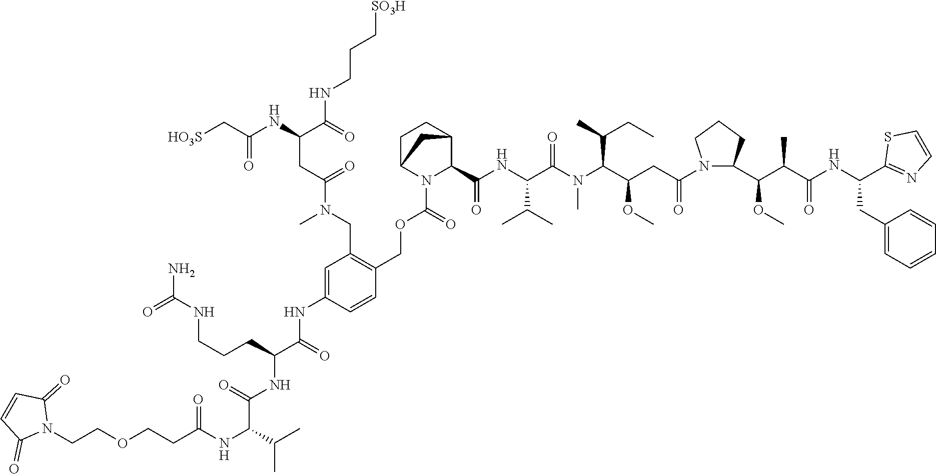 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@@H](NC(=O)CS(=O)(=O)O)C(=O)NCCCS(=O)(=O)O)C(C)C] |
| L127- P1 | 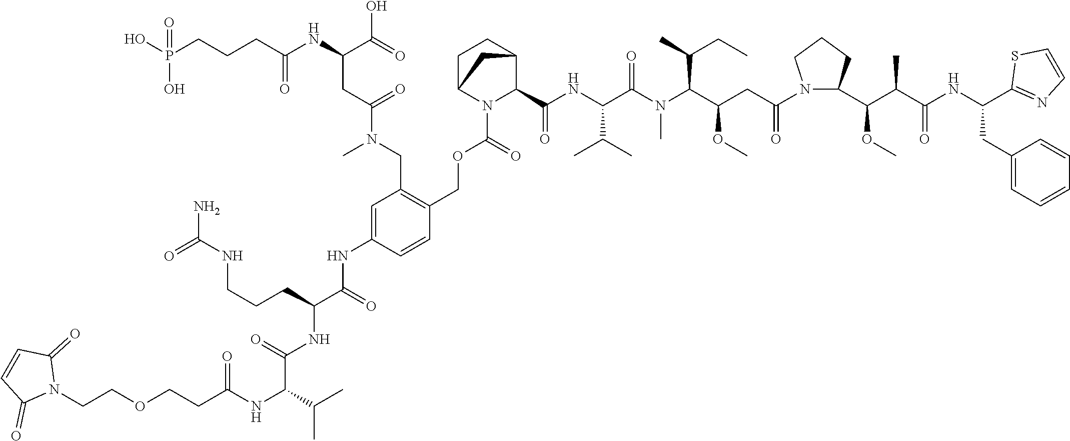 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)C[C@@H](NC(=O)CCCP(=O)(O)O)C(=O)O)C(C)C] |
| L128- P1 | 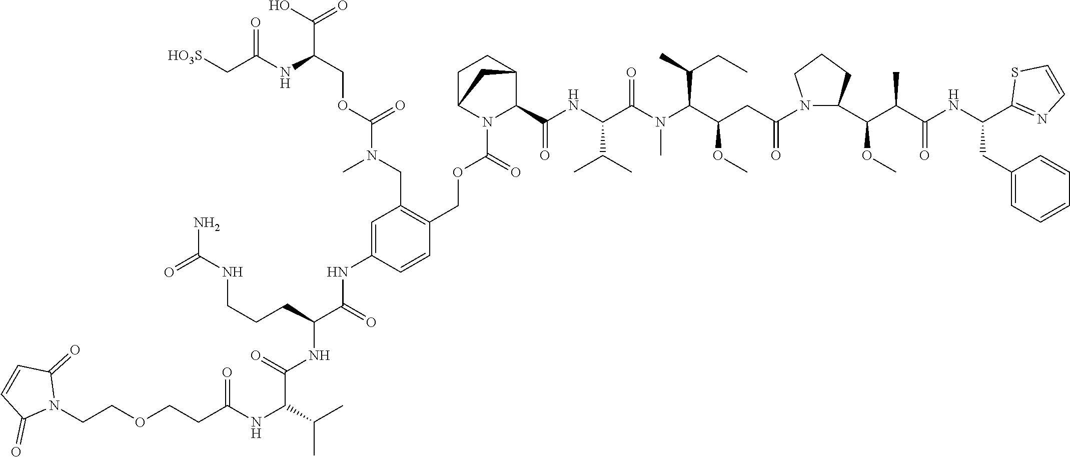 |
| L129- P1 | 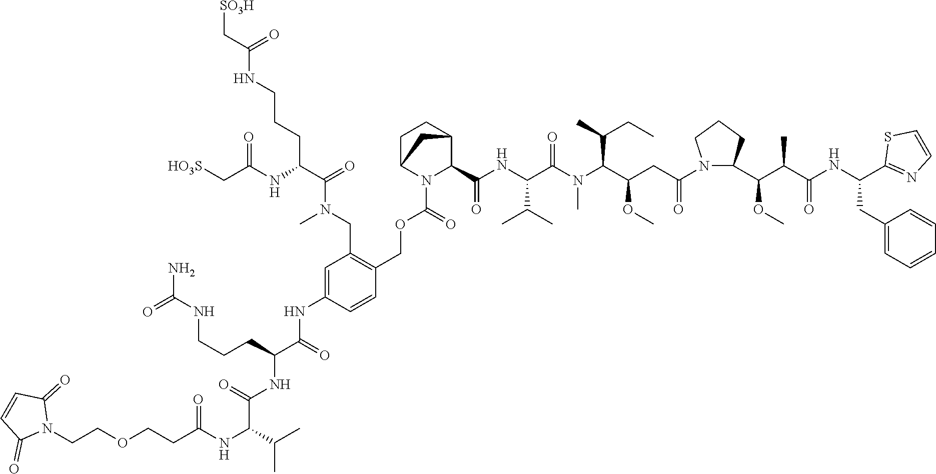 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](CCCNC(=O)CS(=O)(=O)O)NC(=O)CS(=O)(=O)O)C(C)C] |
| L130- P1 | 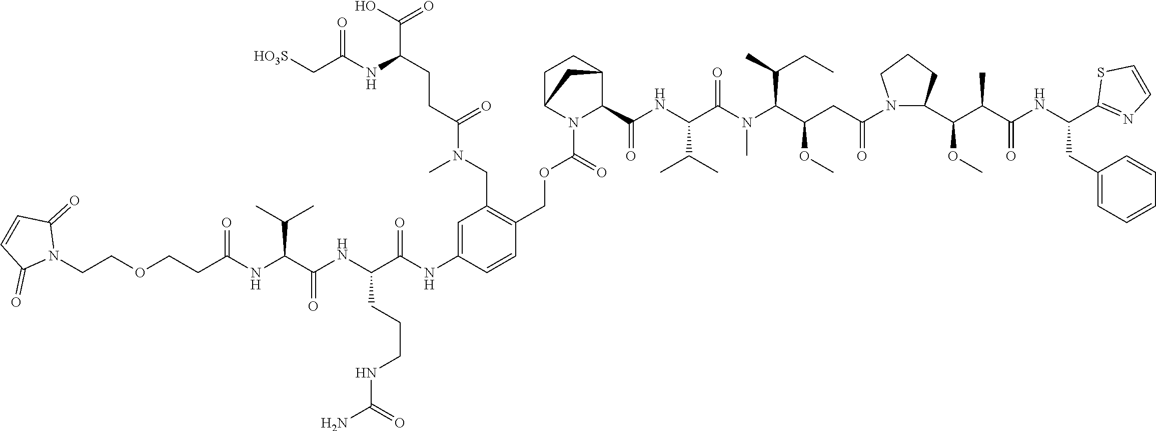 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CC[C@@H](NC(=O)CS(=O)(=O)O)C(=O)O)C(C)C] |
| L131- P1 | 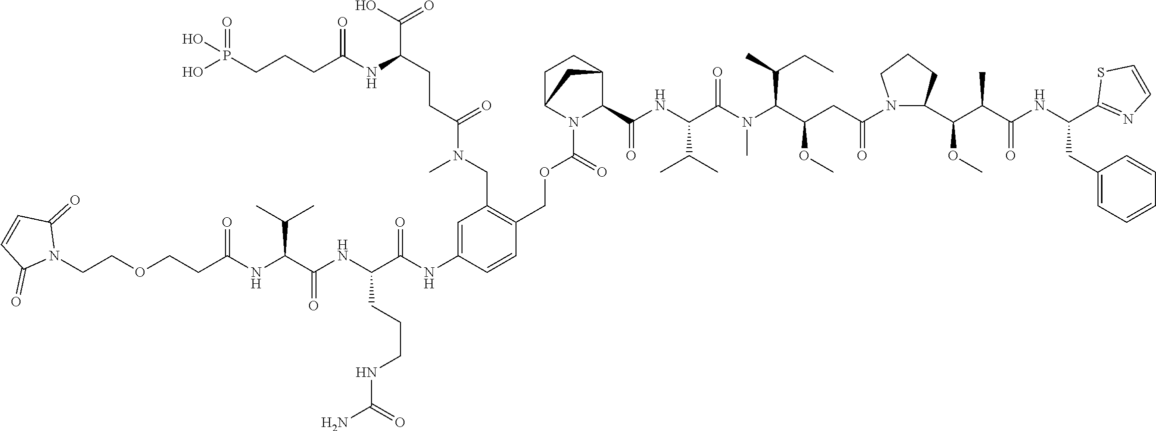 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CC[C@@H](NC(=O)CCCP(=O)(O)O)C(=O)O)C(C)C] |
| L132- P1 | 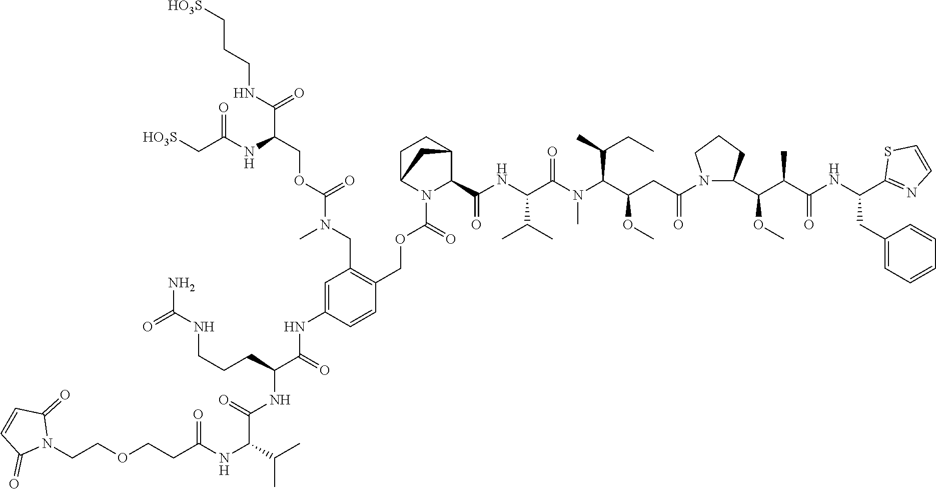 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@@H](NC(=O)CS(=O)(=O)O)C(=O)NCCCS(=O)(=O)O)C(C)C] |
| L133- P1 | 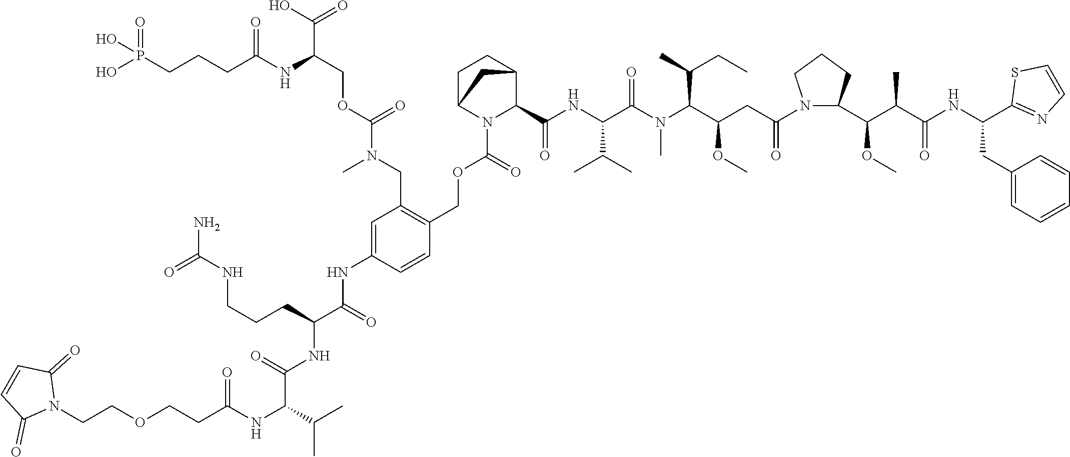 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@@H](NC(=O)CCCP(=O)(O)O)C(=O)O)C(C)C] |
| L134- P1 | 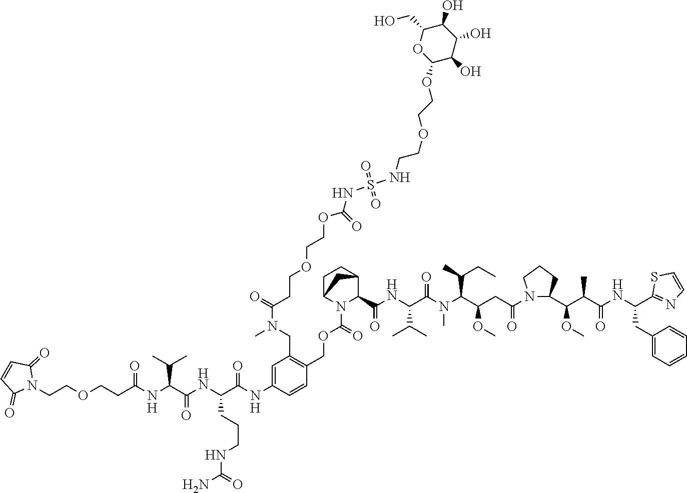 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOC(=O)NS(=O)(=O)NCCOCCO[C@@H]1O[C@H](CO)[C@@H](O)[C@H](O)[C@H]1O)C(C)C] |
| L135- P1 | 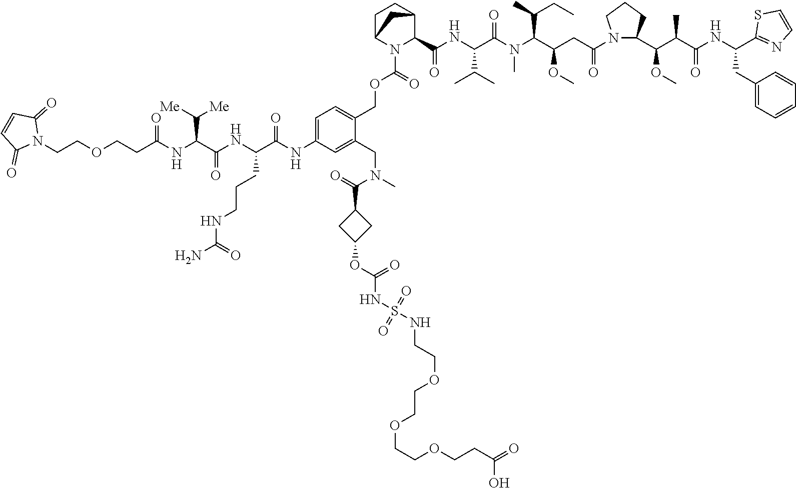 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCOCCC(=O)O)C1)C(C)C] |
| L136- P1 | 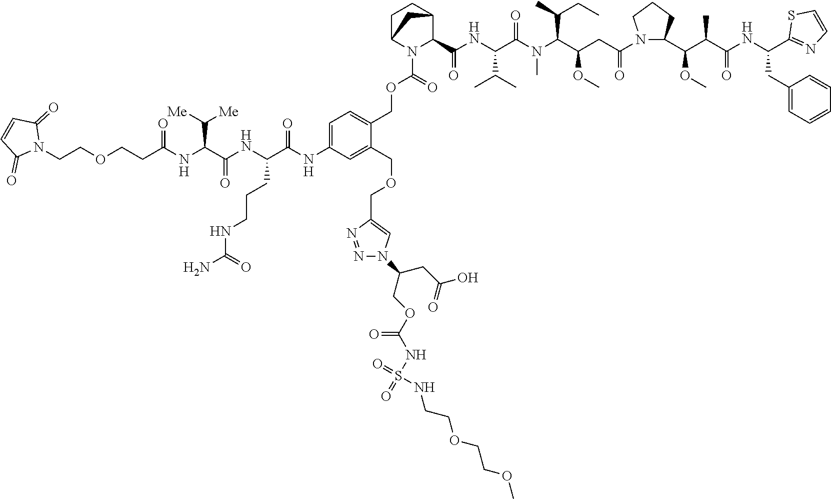 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)NS(=O)(=O)NCCOCCOC)CC(=O)O)nn1)C(C)C] |

TABLE 4C

| Com- | |
|---|---|
| pound | |
| Num- | |
| ber | Structure |
| L137- P2 | 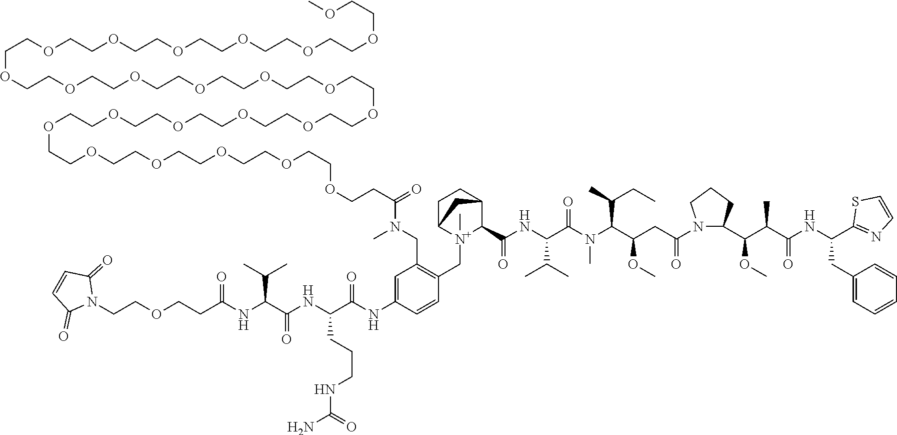 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC)C(C)C] |
| L138- P2 | 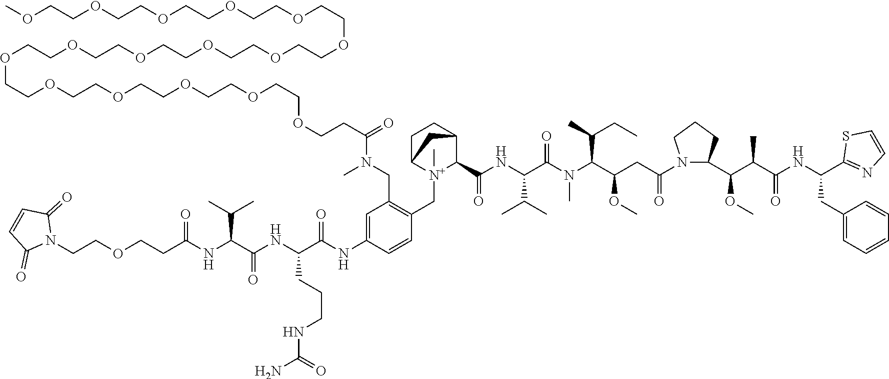 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOC)C(C)C] |
| L139- P2 | 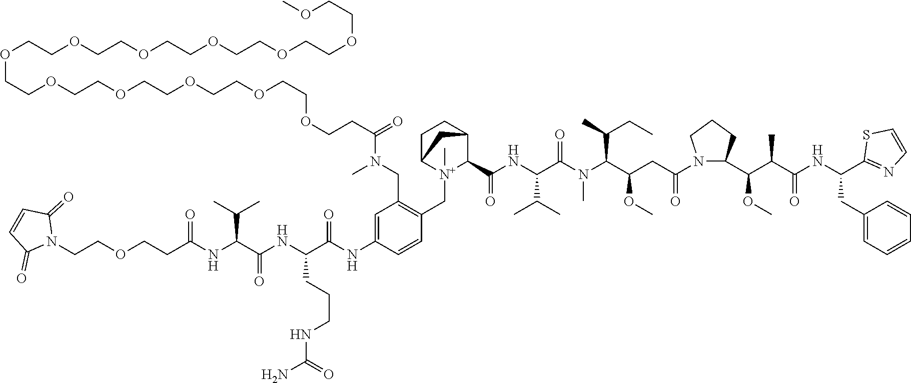 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOC)C(C)C] |
| L140- P2 | 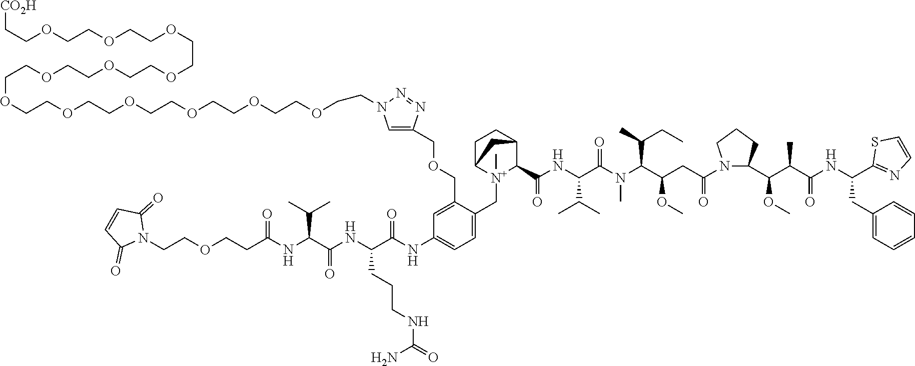 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCOCCOCCOCCOCCC(=O)O)nn1)C(C)C] |
| L141- P2 | 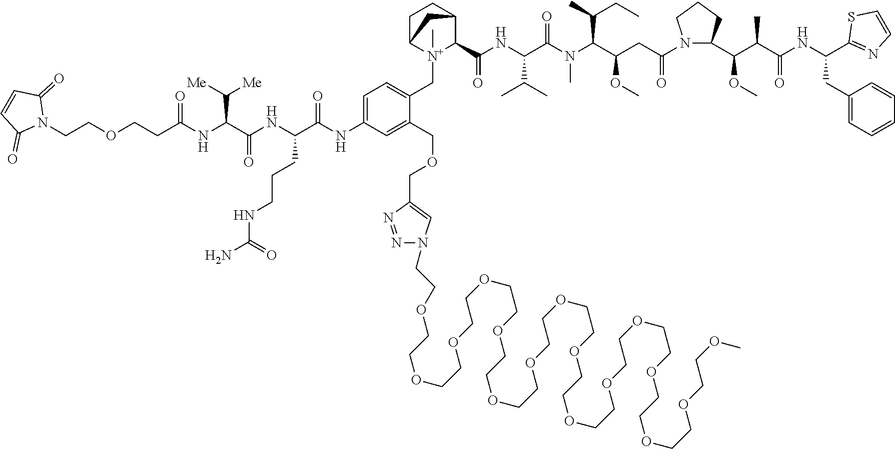 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCOCCOCCOCCOCCOCCOCCOCCOC)nn1)C(C)C] |
| L142- P2 | 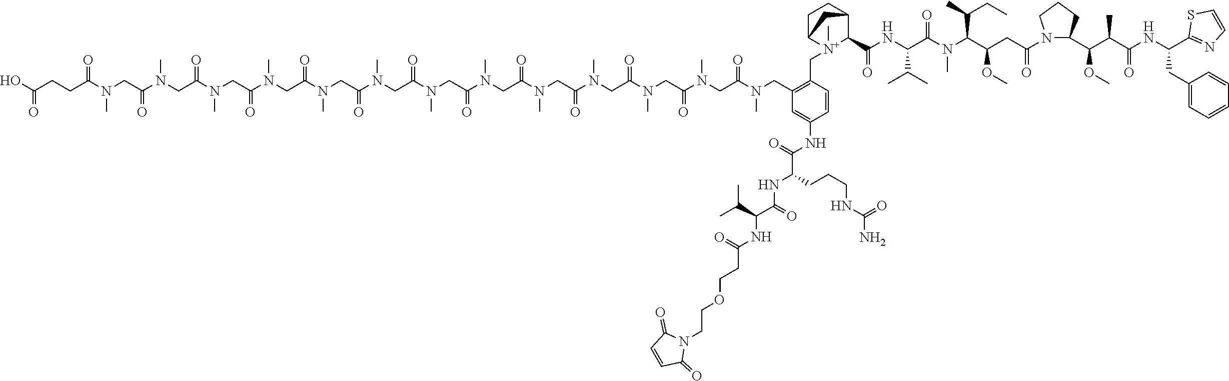 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)C(C)C] |
| L143- P2 | 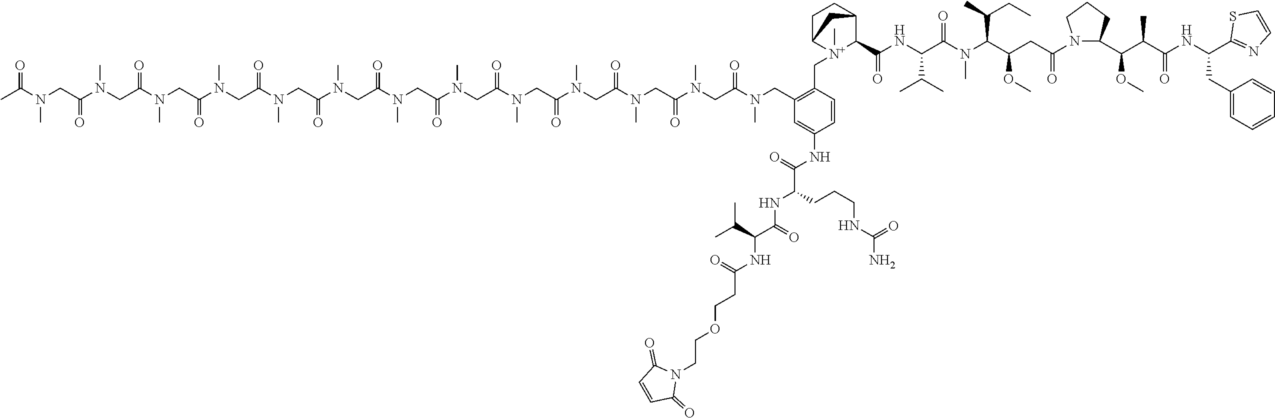 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)C(C)C] |
| L144- P2 | 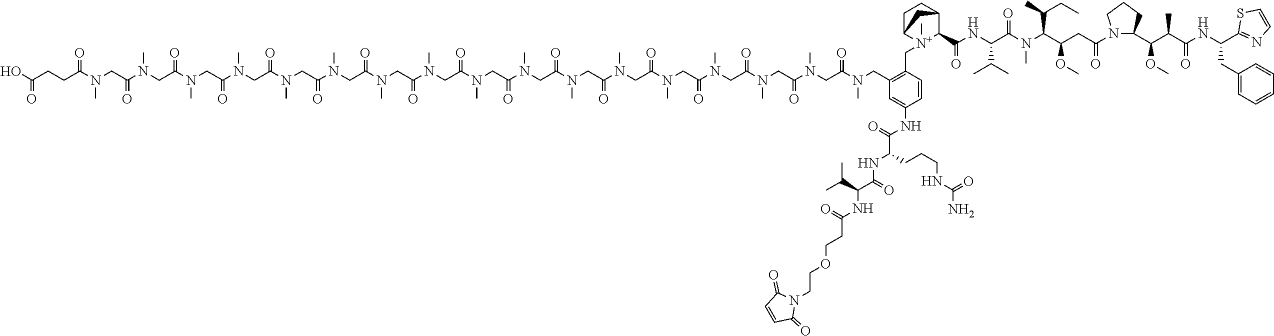 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)C(C)C] |
| L145- P2 | 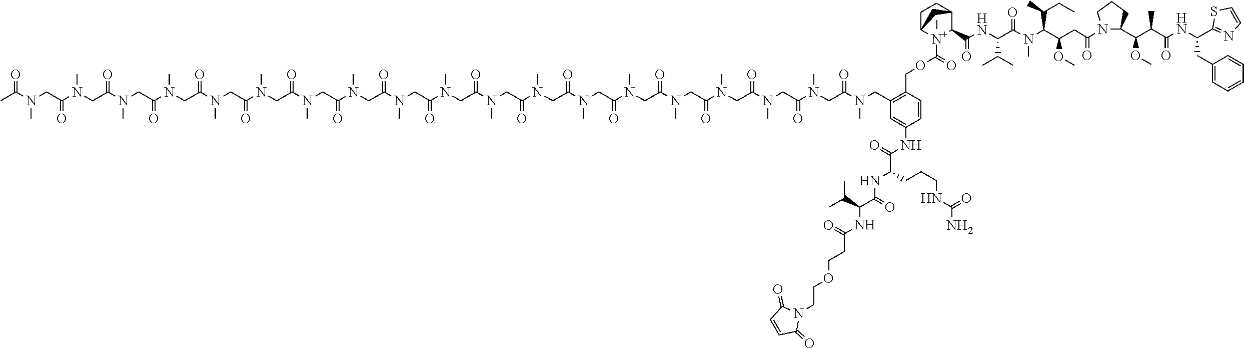 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)C(C)C] |
| L146- P2 | 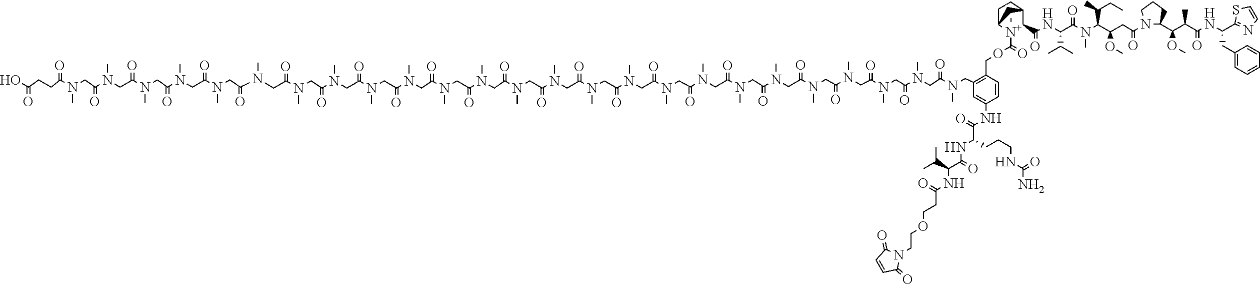 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)C(C)C] |
| L147- P2 | 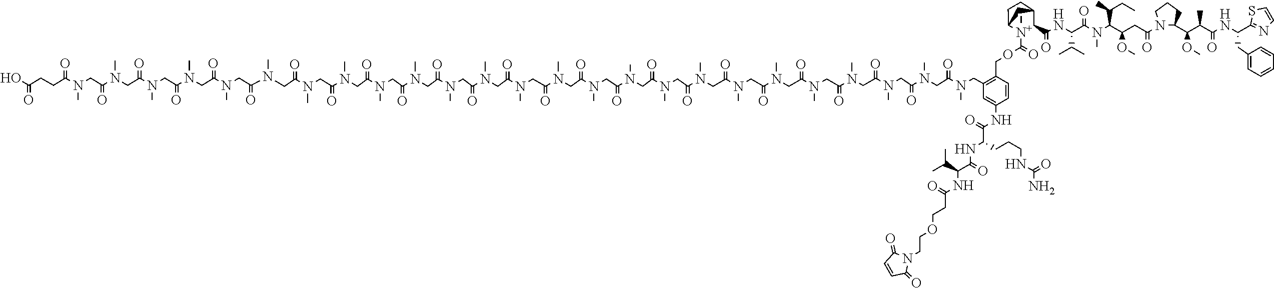 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)C(C)C] |
| L148- P2 | 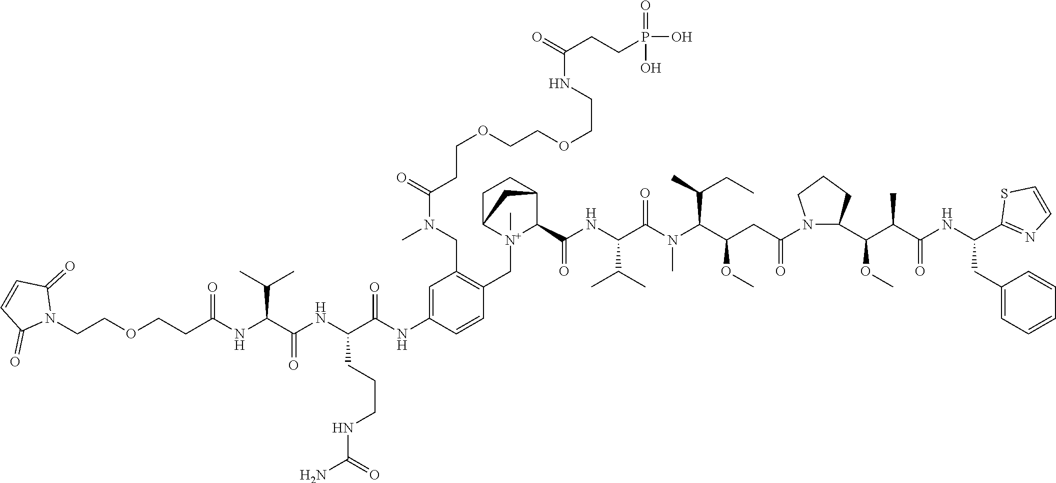 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCNC(=O)CCP(=O)(O)O)C(C)C] |
| L149- P2 | 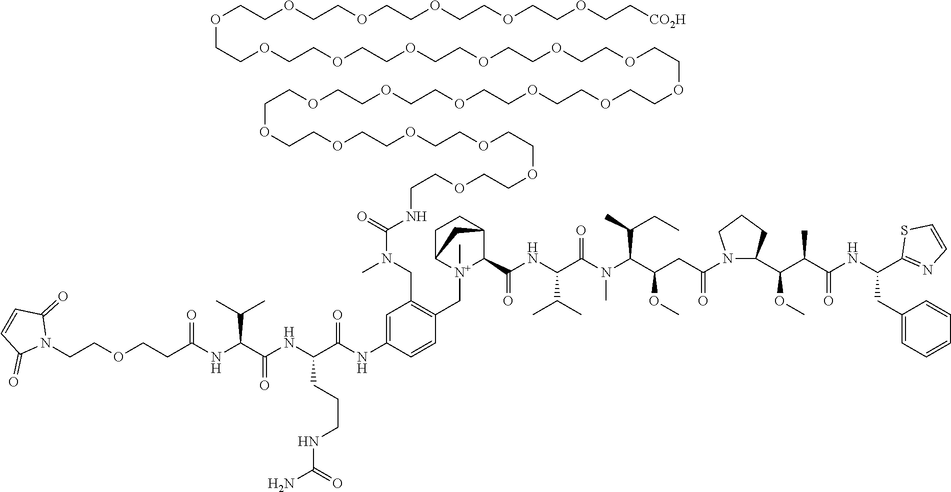 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)C(C)C] |
| L150- P2 | 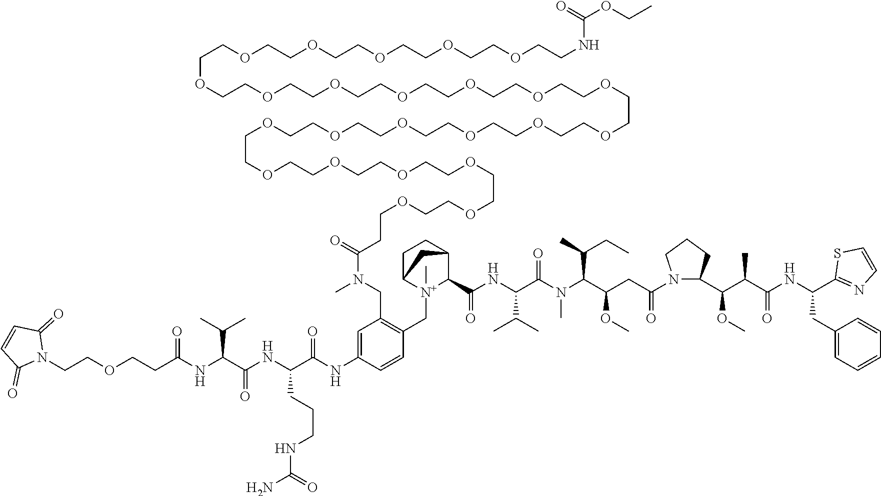 [SMILES: CCOC(=O)NCCOCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(C)[C@@H]2CC[C@@H](C2)[C@H]1C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C] |
| L151- P2 | 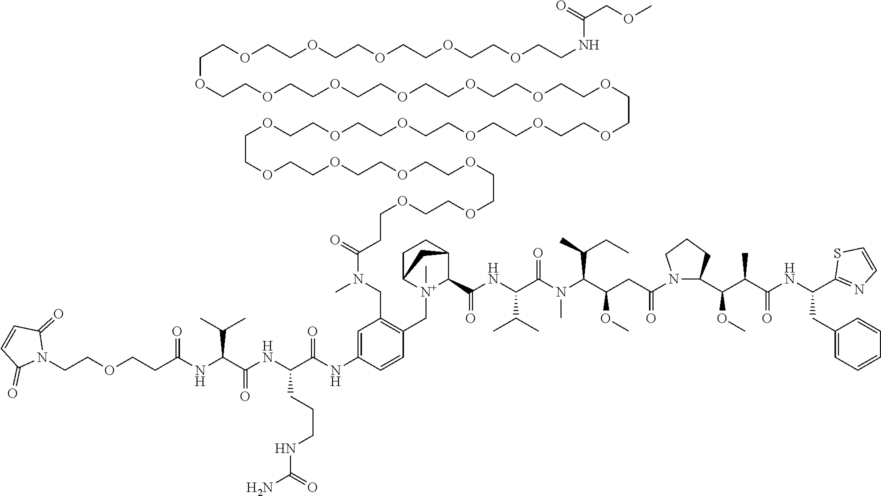 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCNC(=O)COC)C(C)C] |
| L152- P2 | 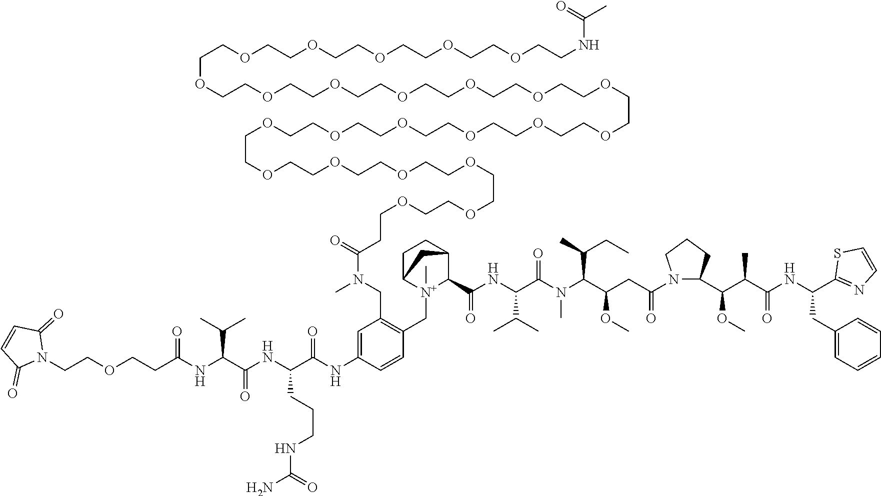 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCNC(C)=O)C(C)C] |
| L153- P2 | 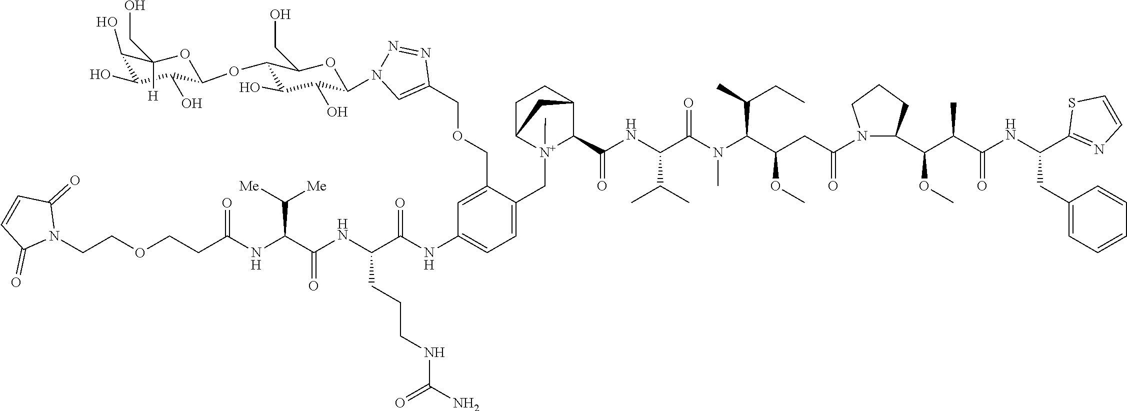 |
| L154- P2 | 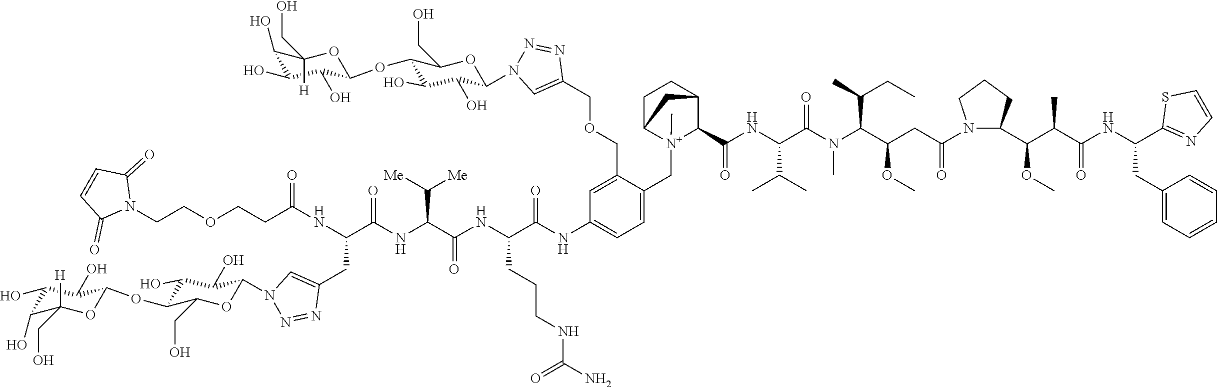 |
| L155- P2 | 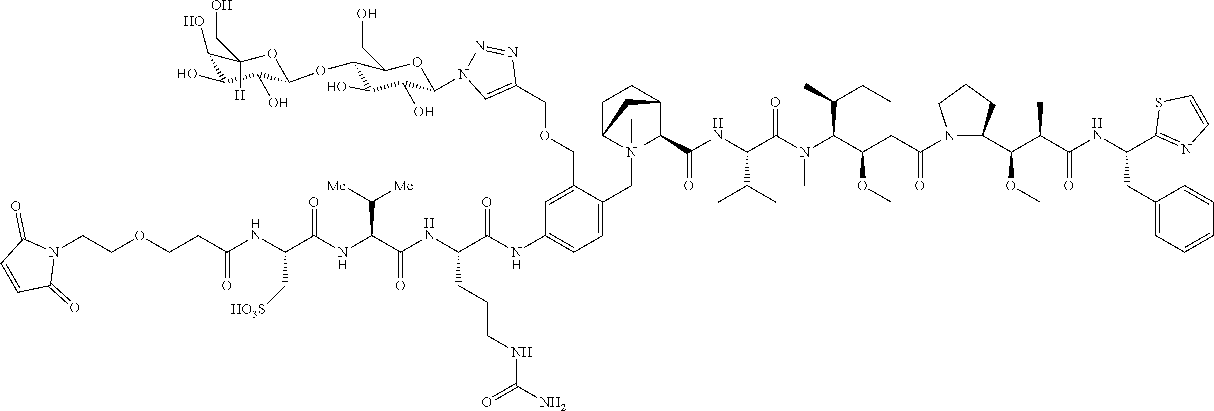 |
| L156- P2 | 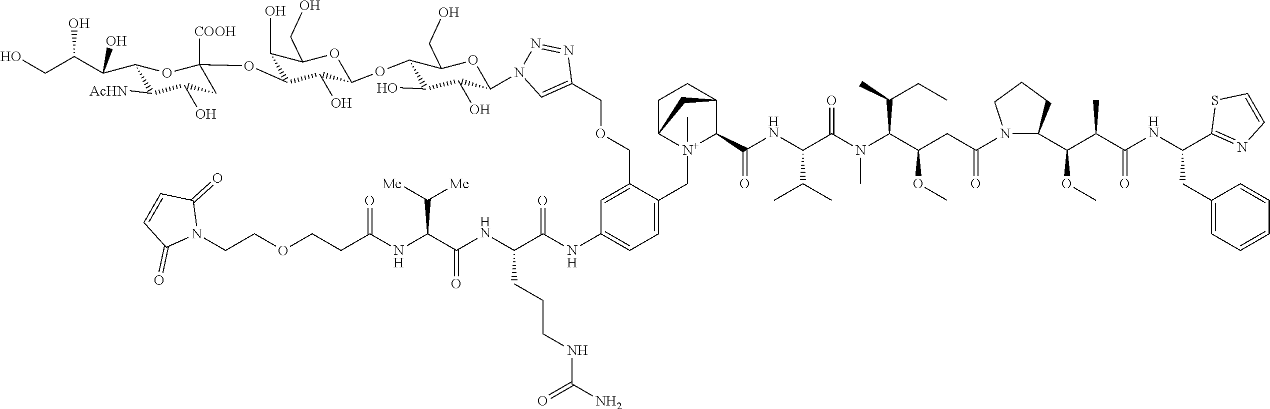 |
| L157- P2 | 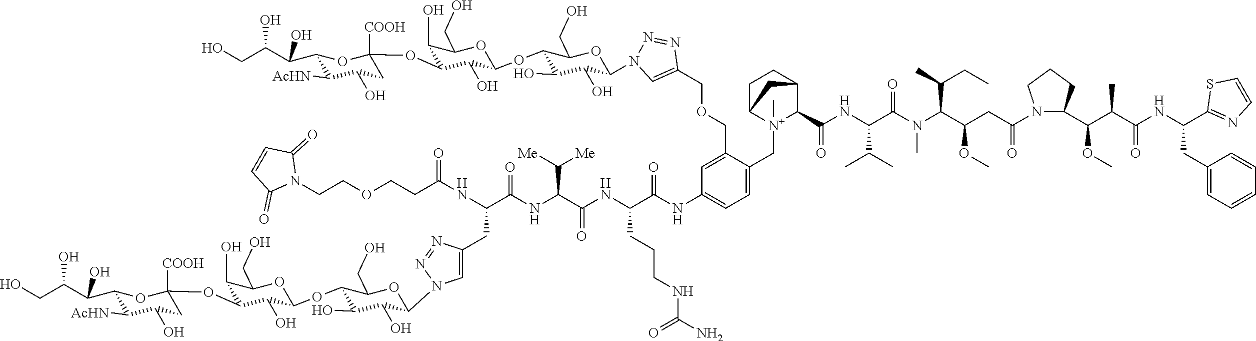 |
| L158- P2 | 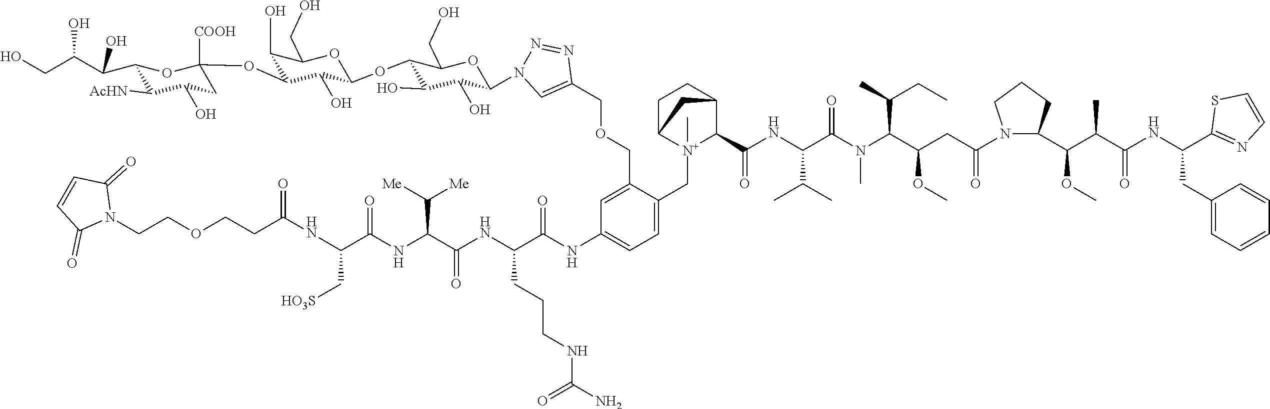 |
| L159- P2 | 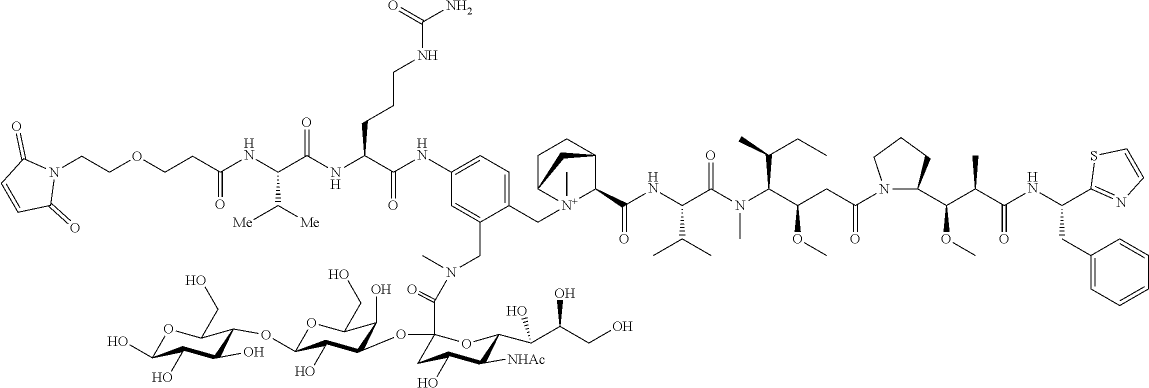 |
| L160- P2 | 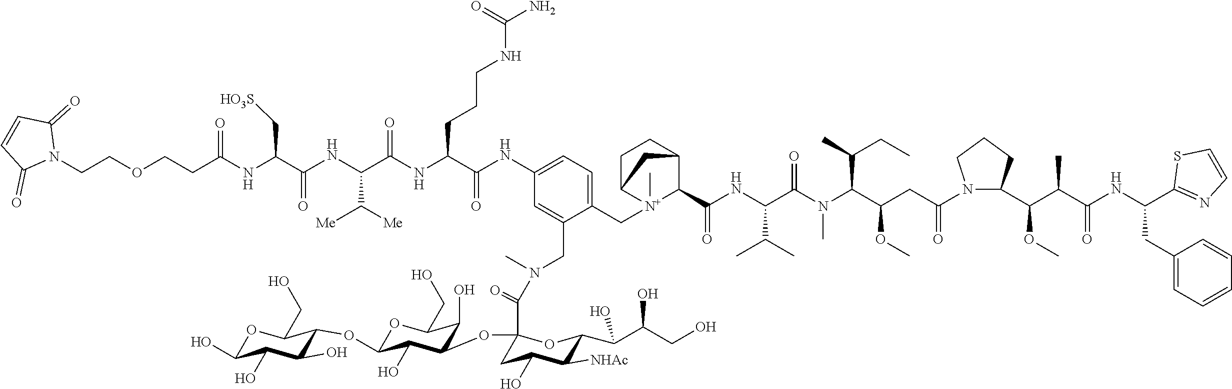 |
| L161- P2 | 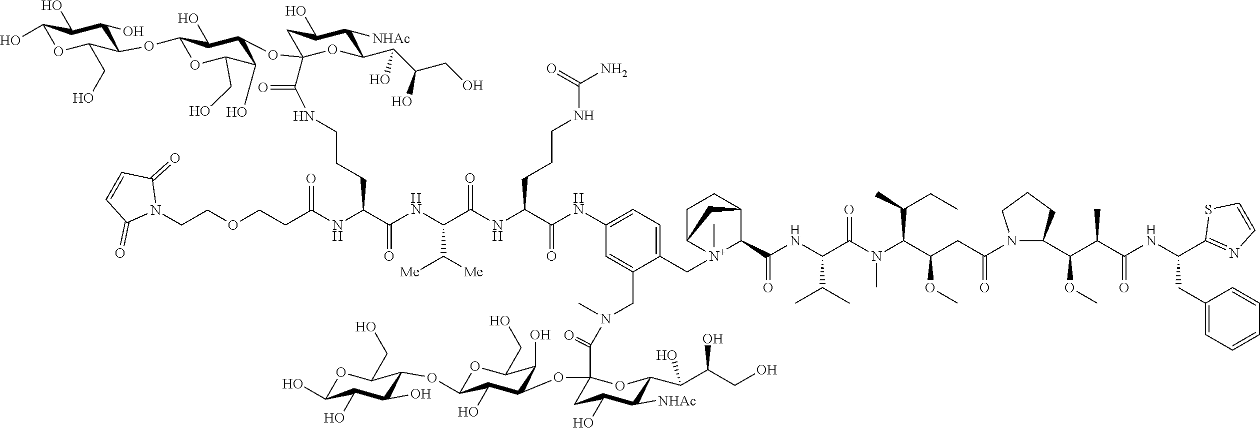 |
| L162- P2 | 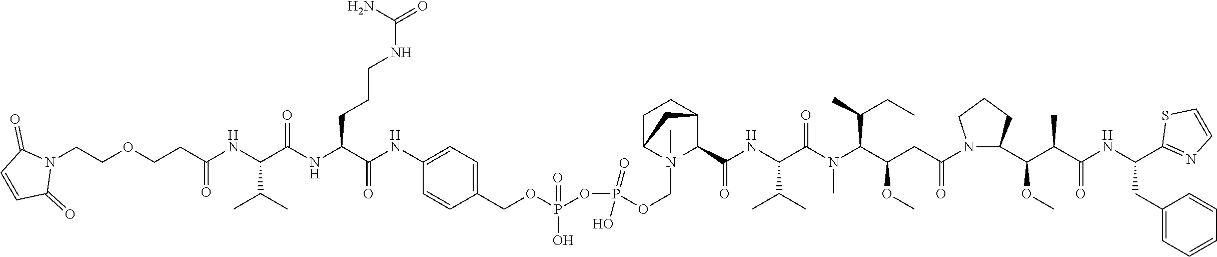 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)COP(=O)(O)OP(=O)(O)OCc1ccc(NC(=O)[C@H](CCCNC(N)=O)NC(=O)[C@@H](NC(=O)CCOCCN2C(=O)C=CC2=O)C(C)C)cc1)C(C)C] |
| L163- P2 | 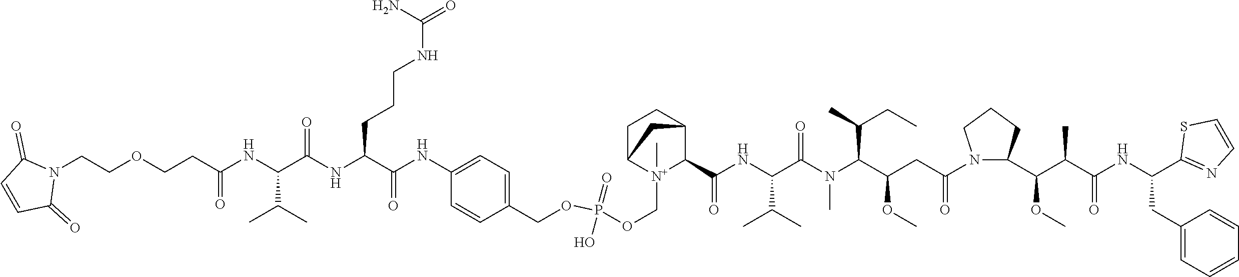 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)COP(=O)(O)OCc1ccc(NC(=O)[C@H](CCCNC(N)=O)NC(=O)[C@@H](NC(=O)CCOCCN2C(=O)C=CC2=O)C(C)C)cc1)C(C)C] |
| L164- P2 | 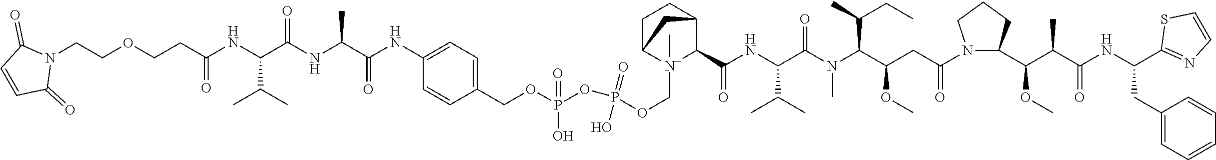 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)COP(=O)(O)OP(=O)(O)OCc1ccc(NC(=O)[C@H](C)NC(=O)[C@@H](NC(=O)CCOCCN2C(=O)C=CC2=O)C(C)C)cc1)C(C)C] |
| L165- P2 | 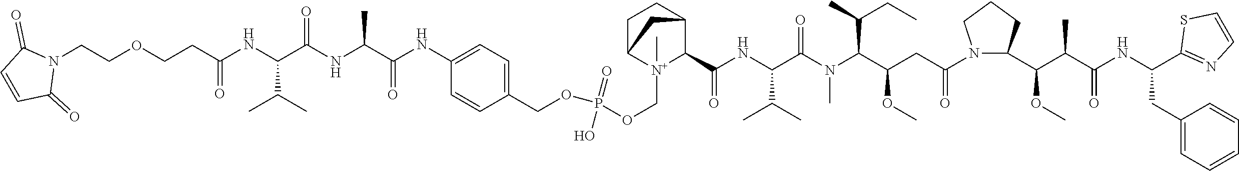 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)COP(=O)(O)OCc1ccc(NC(=O)[C@H](C)NC(=O)[C@@H](NC(=O)CCOCCN2C(=O)C=CC2=O)C(C)C)cc1)C(C)C] |
| L166- P2 | 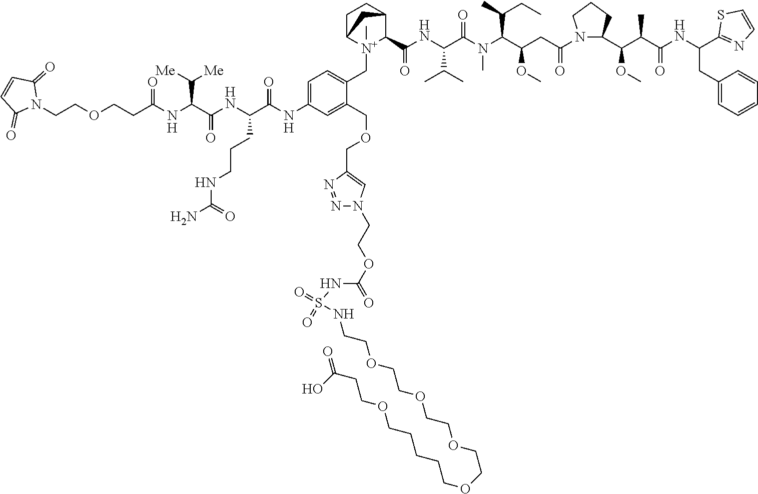 [SMILES: CC[C@H](C)[C@@H]([C@@H](CC(=O)N1CCC[C@H]1[C@H](OC)[C@@H](C)C(=O)NC(Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCOCCOCCOCCCCCOCCC(=O)O)nn1)C(C)C] |
| L167- P2 | 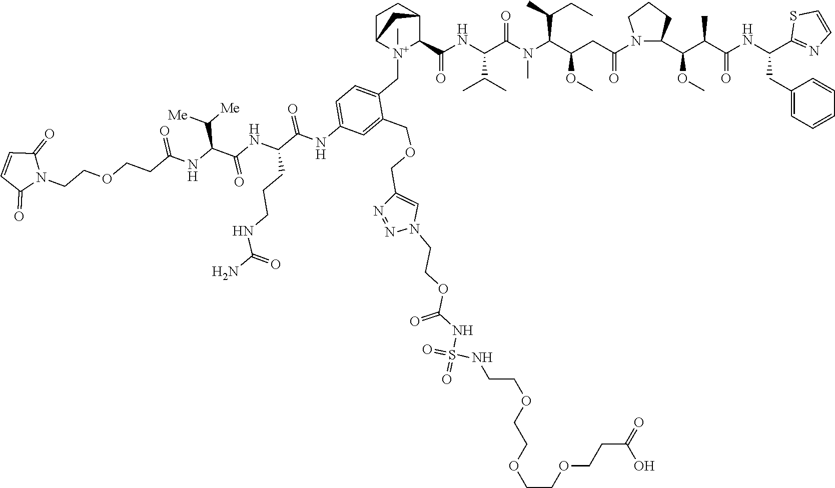 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCOCCOCCC(=O)O)nn1)C(C)C] |
| L168- P2 | 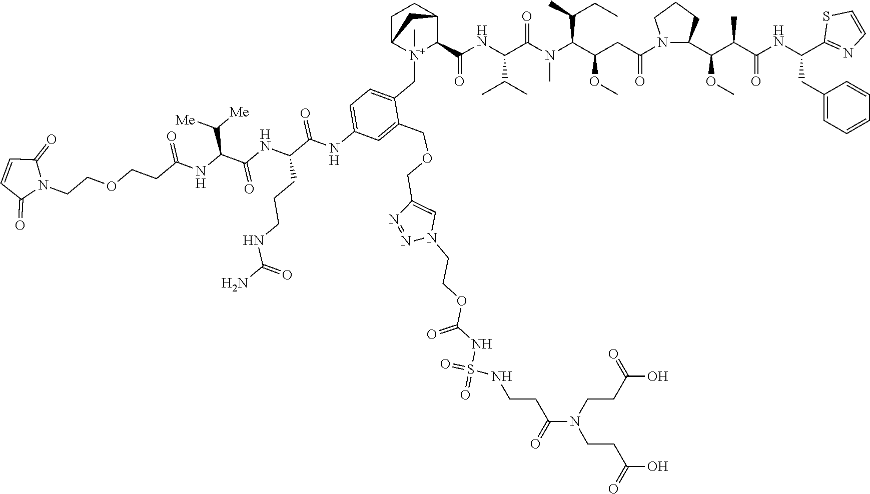 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCC(=O)N(CCC(=O)O)CCC(=O)O)nn1)C(C)C] |
| L169- P2 | 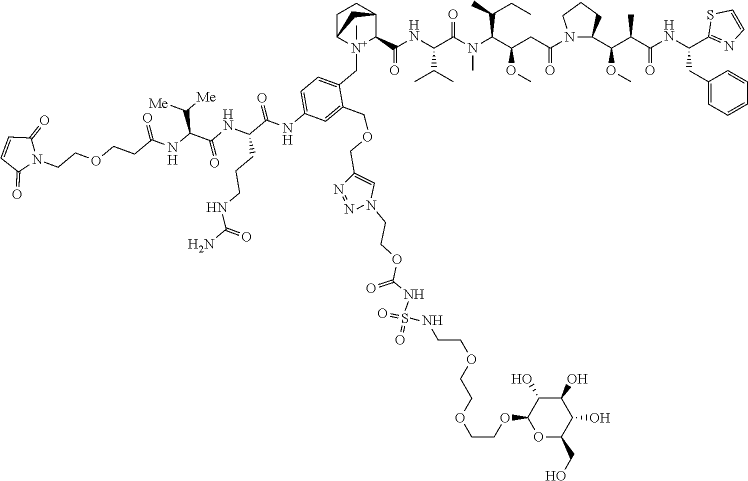 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCOCCO[C@@H]2O[C@H](CO)[C@@H](O)[C@H](O)[C@H]2O)nn1)C(C)C] |
| L170- P2 | 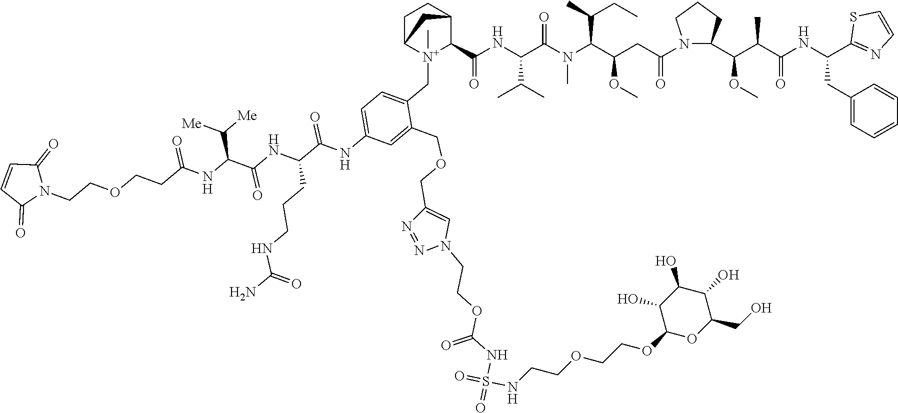 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCO[C@@H]2O[C@H](CO)[C@@H](O)[C@H](O)[C@H]2O)nn1)C(C)C] |
| L171- P2 | 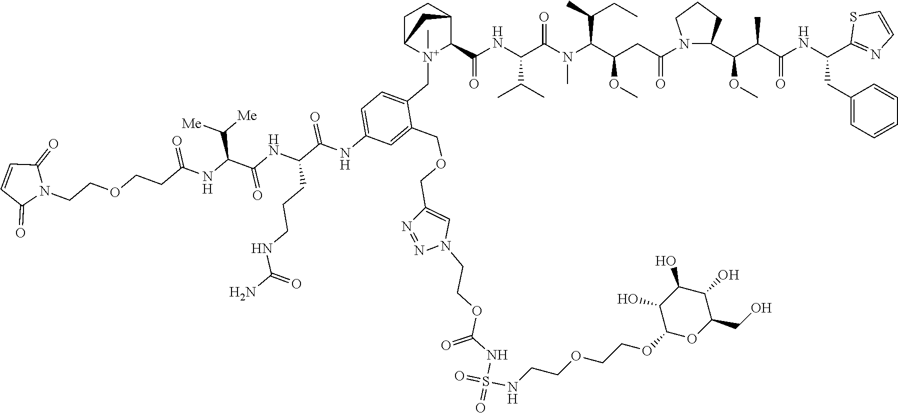 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCO[C@H]2O[C@H](CO)[C@@H](O)[C@H](O)[C@H]2O)nn1)C(C)C] |
| L172- P2 | 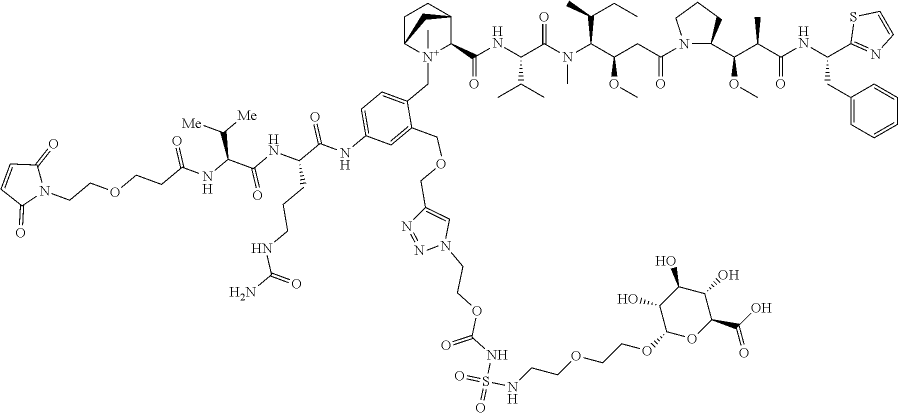 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCOCCO[C@H]2O[C@H](C(=O)O)[C@@H](O)[C@H](O)[C@H]2O)nn1)C(C)C] |
| L173- P2 | 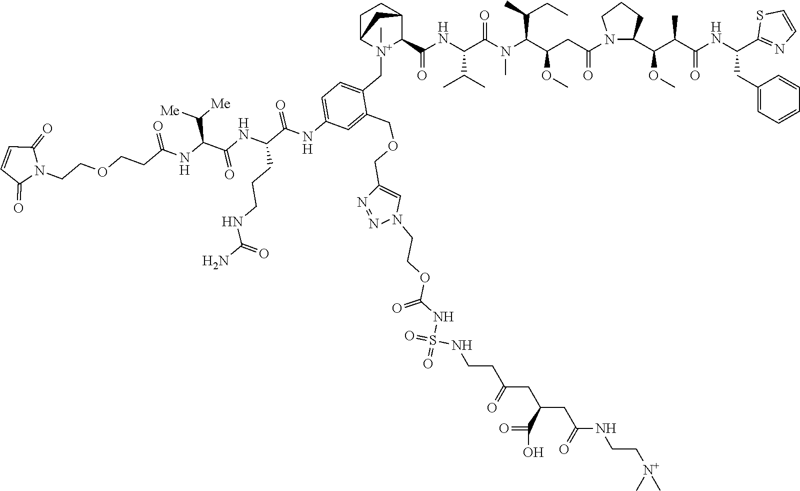 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn(CCOC(=O)NS(=O)(=O)NCCC(=O)C[C@@H](CC(=O)NCC[NH+](C)C)C(=O)O)nn1)C(C)C] |
| L174- P2 | 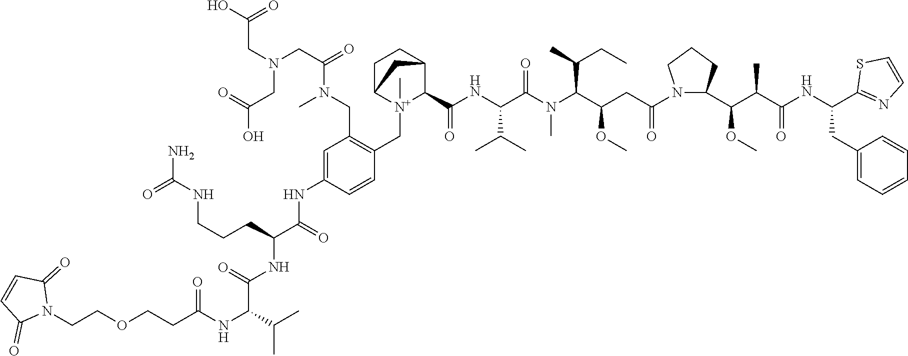 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(CC(=O)O)CC(=O)O)C(C)C] |
| L175- P2 | 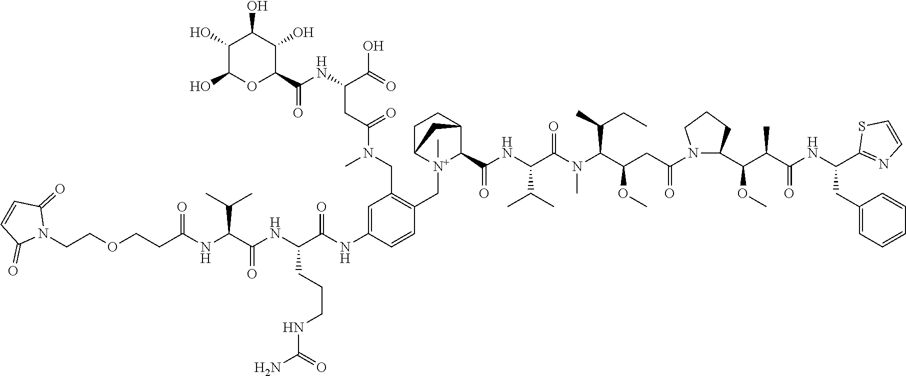 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)[C@H]1O[C@@H](O)[C@H](O)[C@@H](O)[C@@H]1O)C(=O)O)C(C)C] |
| L176- P2 | 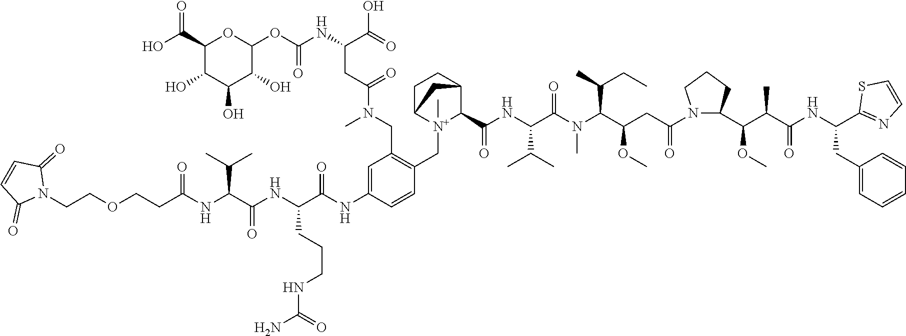 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)OC1O[C@H](C(=O)O)[C@@H](O)[C@H](O)[C@H]1O)C(=O)O)C(C)C] |
| L177- P2 | 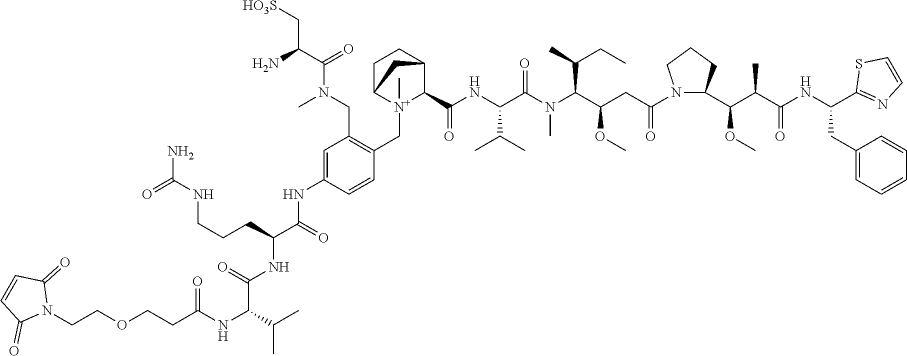 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](N)CS(=O)(=O)O)C(C)C] |
| L178- P2 | 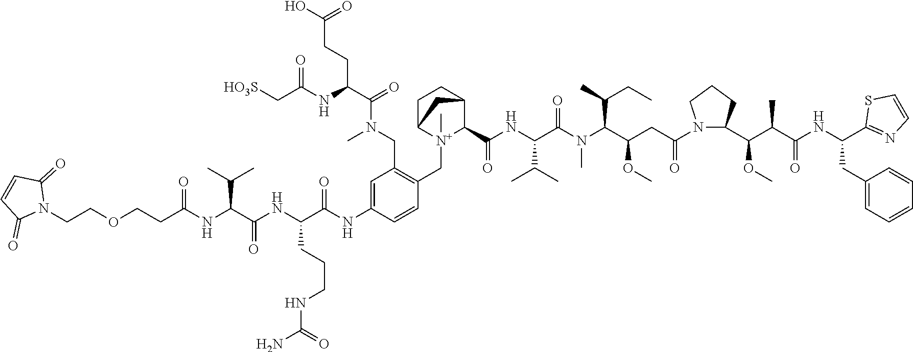 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCC(=O)O)NC(=O)CS(=O)(=O)O)C(C)C] |
| L179- P2 | 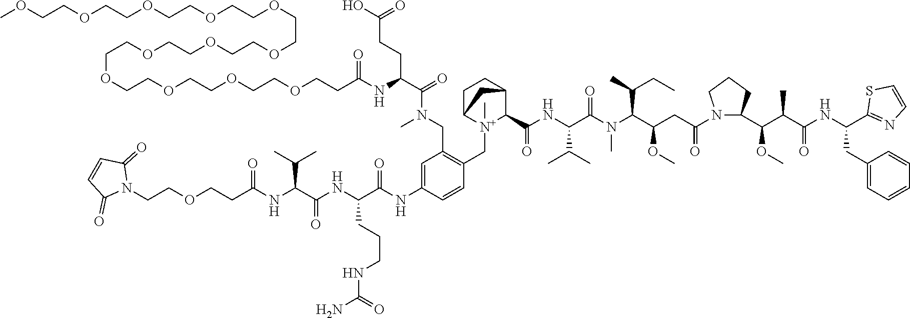 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCC(=O)O)NC(=O)CCOCCOCCOCCOCCOCCOCCOCCOCCOCCOCCOCCOC)C(C)C] |
| L180- P2 | 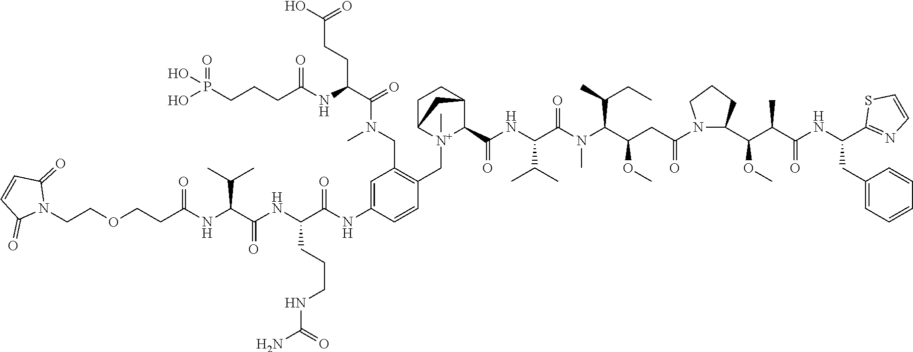 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCC(=O)O)NC(=O)CCCP(=O)(O)O)C(C)C] |
| L181- P2 | 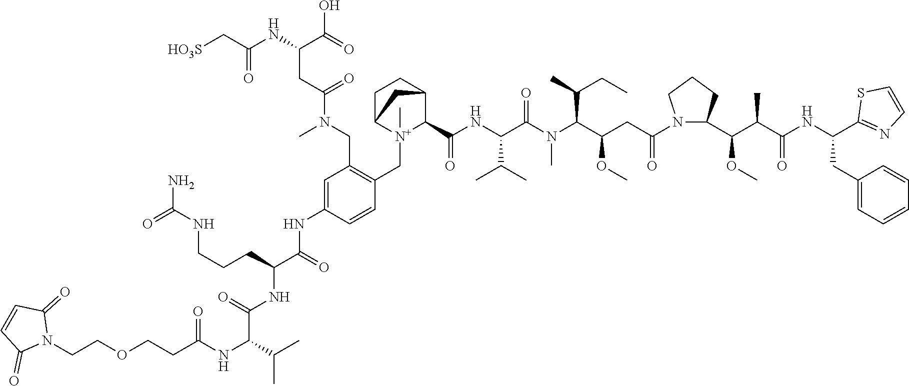 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CS(=O)(=O)O)C(=O)O)C(C)C] |
| L182- P2 | 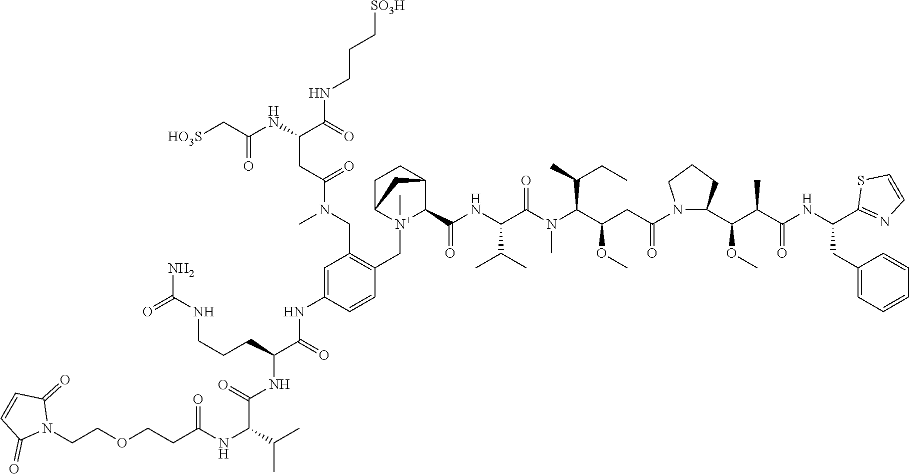 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CS(=O)(=O)O)C(=O)NCCCS(=O)(=O)O)C(C)C] |
| L183- P2 | 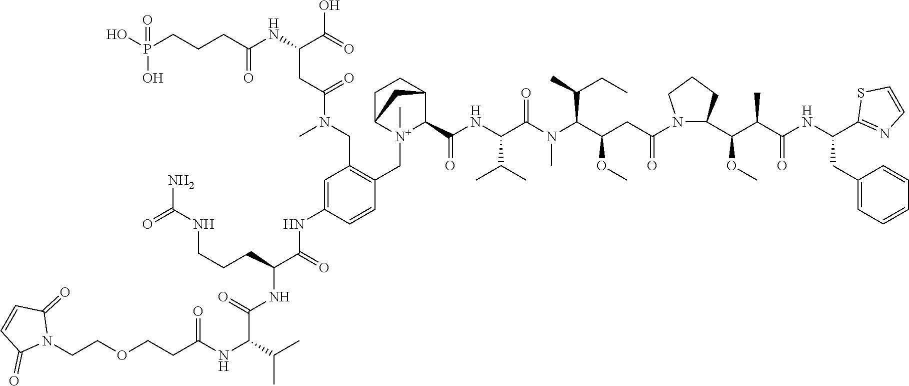 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CCCP(=O)(O)O)C(=O)O)C(C)C] |
| L184- P2 | 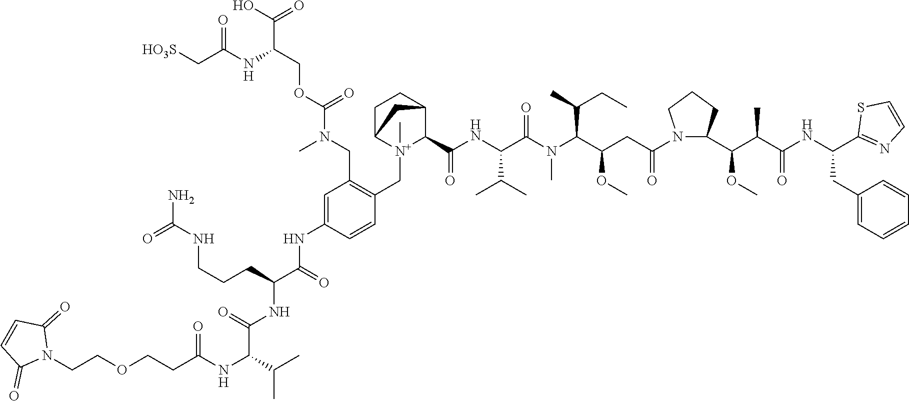 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@H](NC(=O)CS(=O)(=O)O)C(=O)O)C(C)C] |
| L185- P2 | 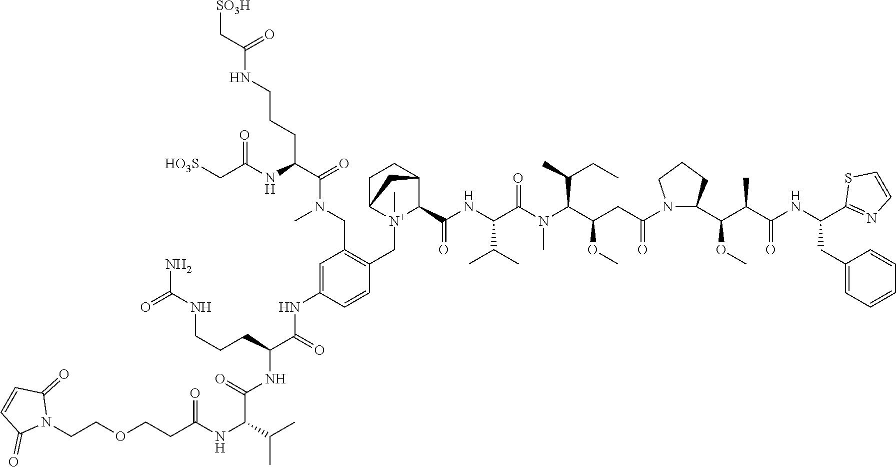 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCCNC(=O)CS(=O)(=O)O)NC(=O)CS(=O)(=O)O)C(C)C] |
| L186- P2 | 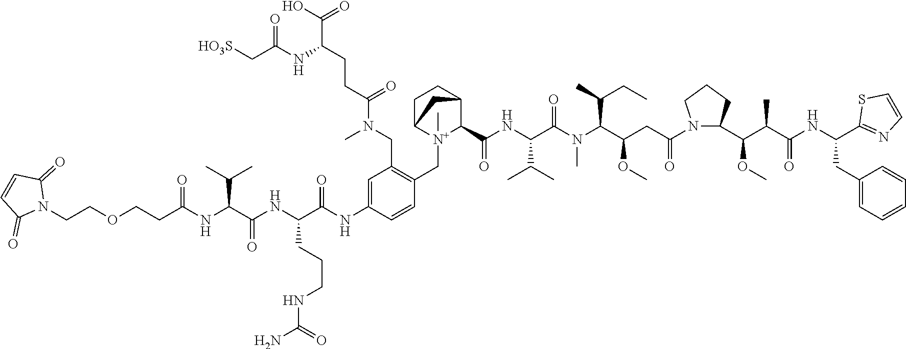 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CC[C@H](NC(=O)CS(=O)(=O)O)C(=O)O)C(C)C] |
| L187- P2 | 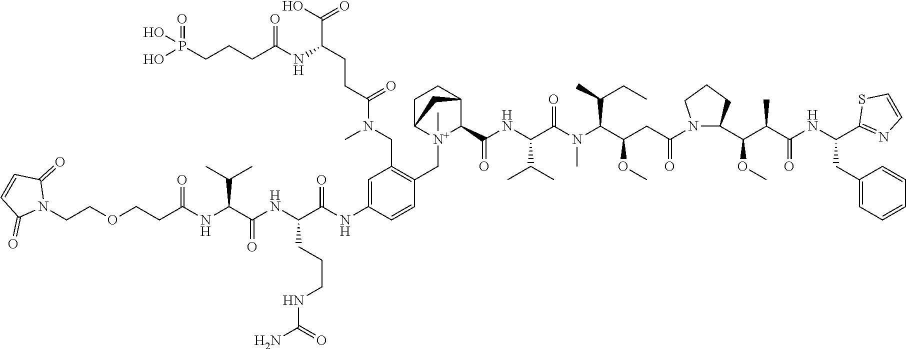 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CC[C@H](NC(=O)CCCP(=O)(O)O)C(=O)O)C(C)C] |
| L188- P2 | 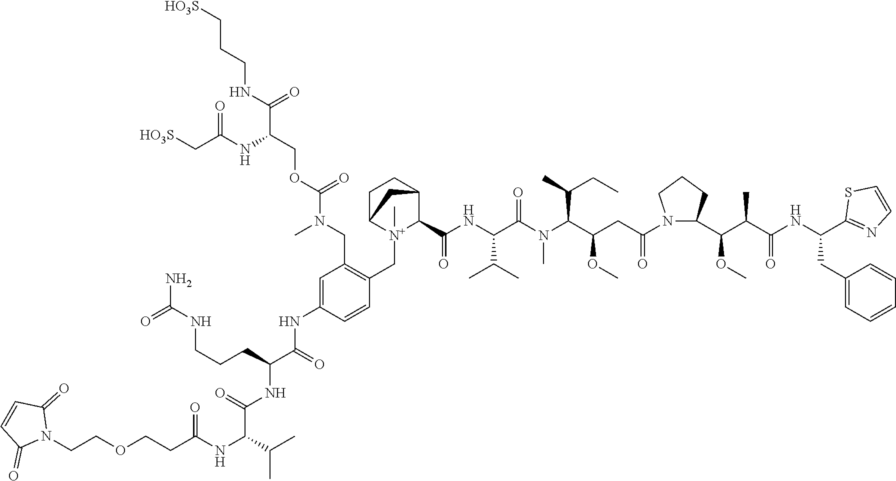 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@H](NC(=O)CS(=O)(=O)O)C(=O)NCCCS(=O)(=O)O)C(C)C] |
| L189- P2 | 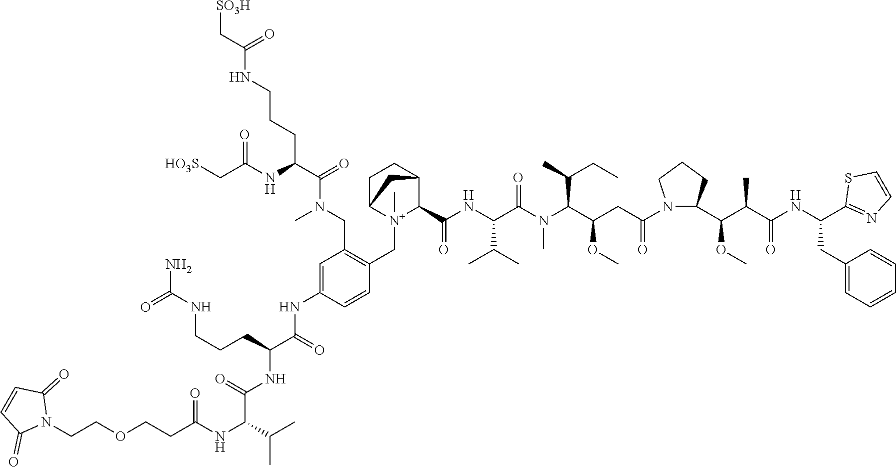 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](CCCNC(=O)CS(=O)(=O)O)NC(=O)CS(=O)(=O)O)C(C)C] |
| L190- P2 | 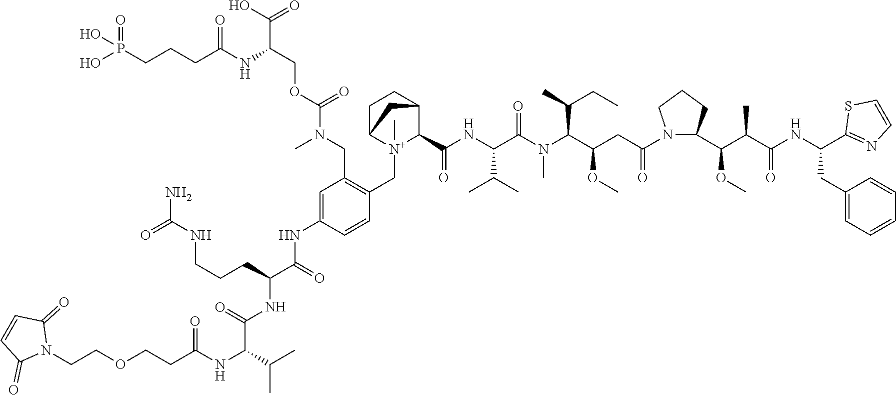 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@H](NC(=O)CCCP(=O)(O)O)C(=O)O)C(C)C] |
| L191- P2 | 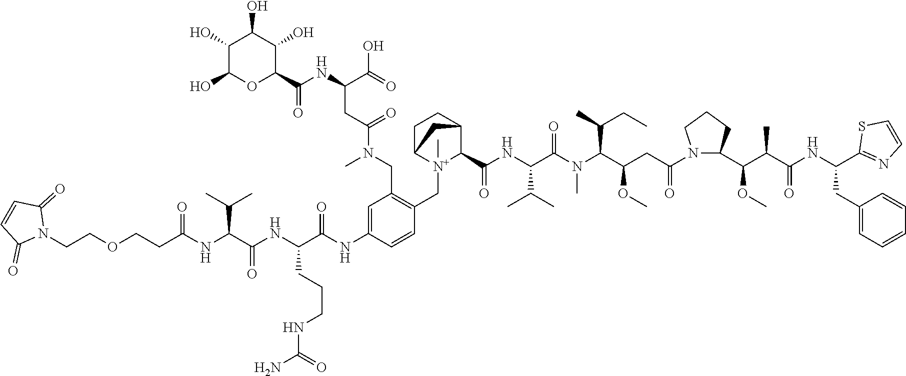 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@@H](NC(=O)[C@H]1O[C@@H](O)[C@H](O)[C@@H](O)[C@@H]1O)C(=O)O)C(C)C] |
| L191- P2 | 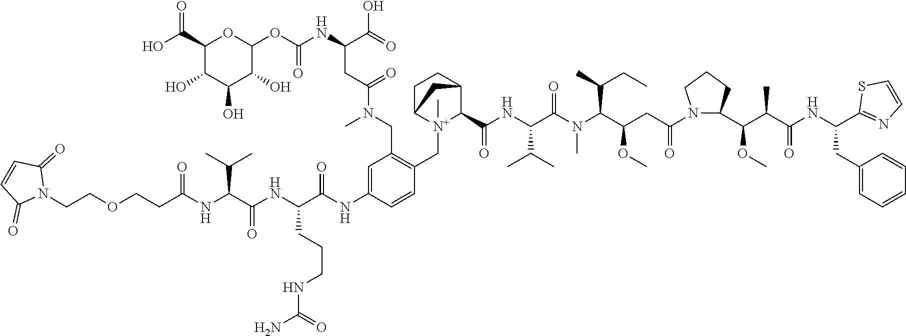 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@@H](NC(=O)OC1O[C@H](C(=O)O)[C@@H](O)[C@H](O)[C@H]1O)C(=O)O)C(C)C] |
| L193- P2 | 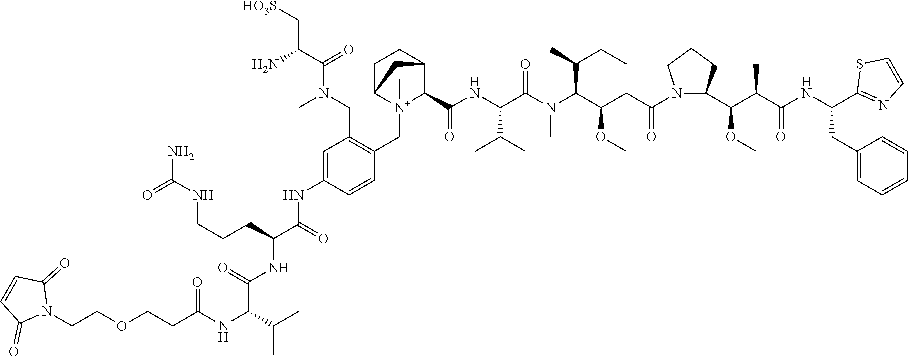 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H](N)CS(=O)(=O)O)C(C)C] |
| L194- P2 | 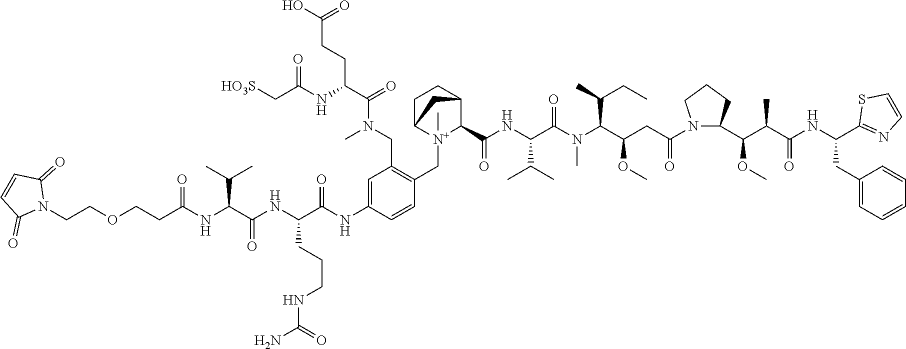 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](CCC(=O)O)NC(=O)CS(=O)(=O)O)C(C)C] |
| L195- P2 | 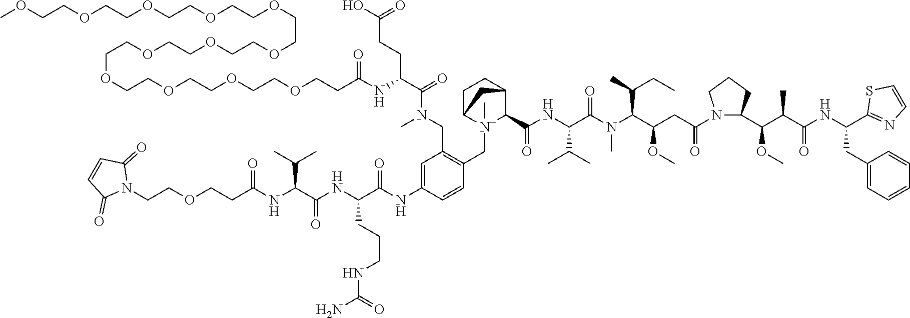 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](CCC(=O)O)NC(=O)CCOCCOCCOCCOCCOCCOCCOCCOCCOCCOCCOCCOC)C(C)C] |
| L196- P2 | 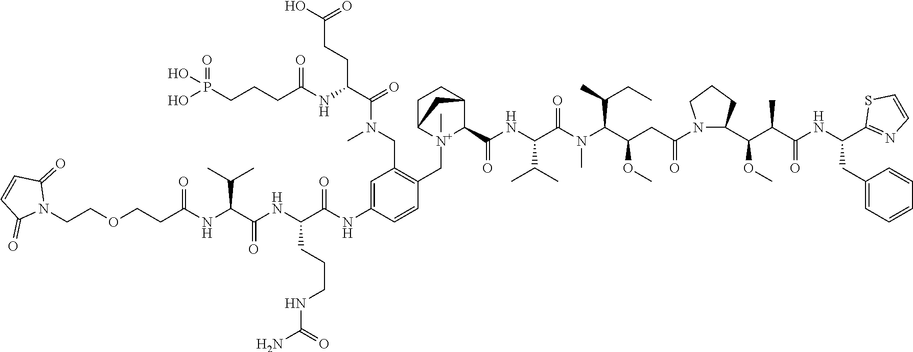 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](CCC(=O)O)NC(=O)CCCP(=O)(O)O)C(C)C] |
| L197- P2 | 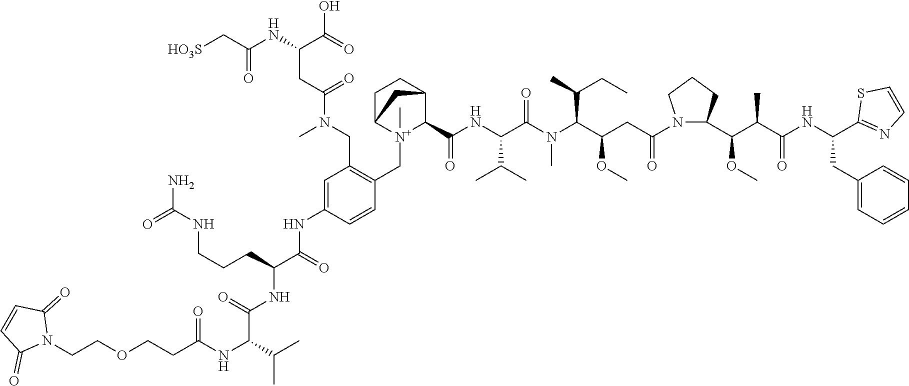 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CS(=O)(=O)O)C(=O)O)C(C)C] |
| L198- P2 | 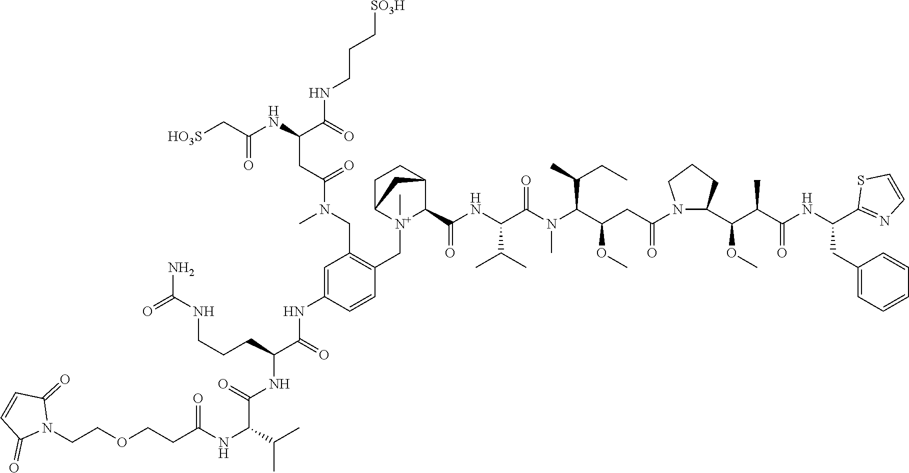 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@@H](NC(=O)CS(=O)(=O)O)C(=O)NCCCS(=O)(=O)O)C(C)C] |
| L199- P2 | 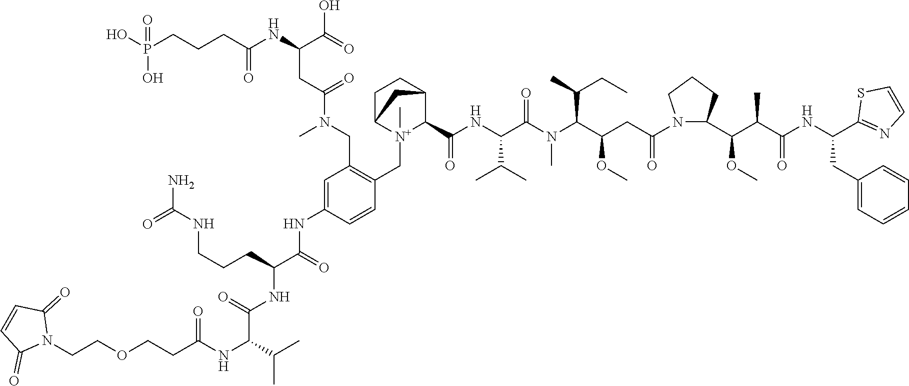 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@@H](NC(=O)CCCP(=O)(O)O)C(=O)O)C(C)C] |
| L200- P2 | 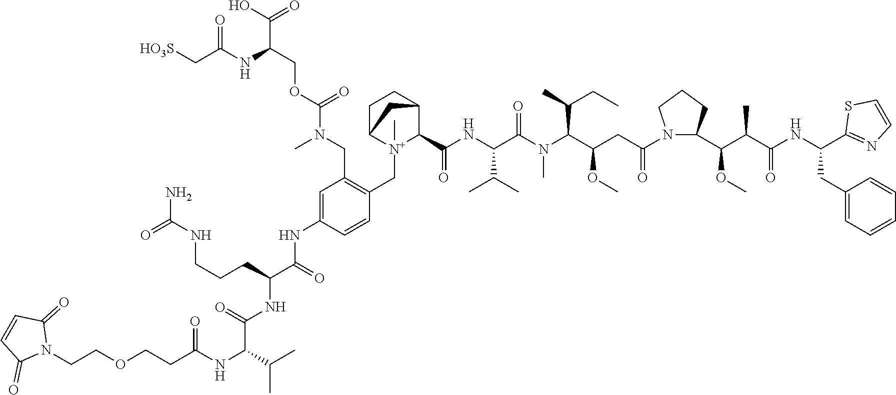 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@@H](NC(=O)CS(=O)(=O)O)C(=O)O)C(C)C] |
| L201- P2 | 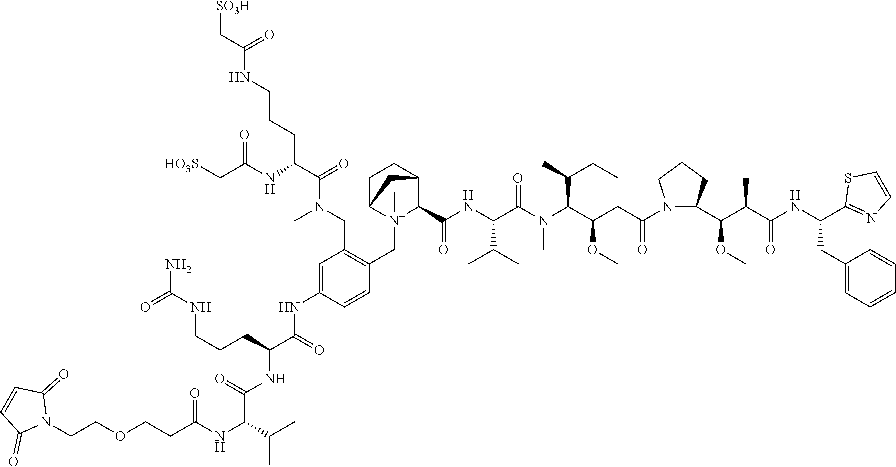 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@@H](CCCNC(=O)CS(=O)(=O)O)NC(=O)CS(=O)(=O)O)C(C)C] |
| L202- P2 | 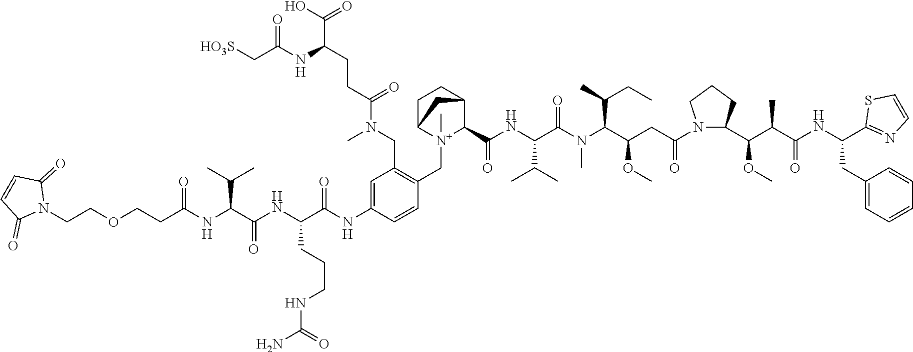 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CC[C@@H](NC(=O)CS(=O)(=O)O)C(=O)O)C(C)C] |
| L203- P2 | 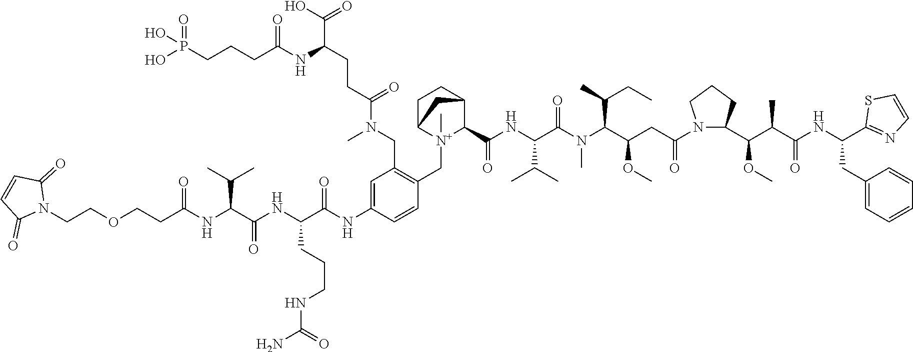 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)CC[C@@H](NC(=O)CCCP(=O)(O)O)C(=O)O)C(C)C] |
| L204- P2 | 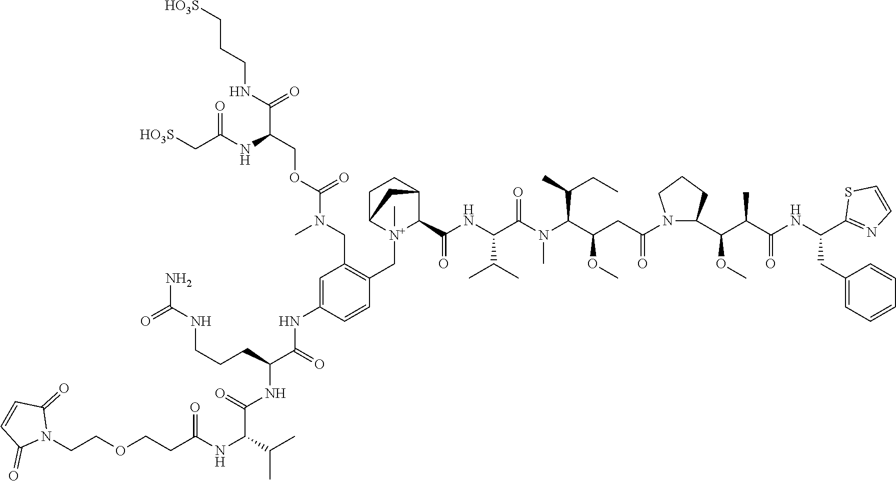 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@@H](NC(=O)CS(=O)(=O)O)C(=O)NCCCS(=O)(=O)O)C(C)C] |
| L205- P2 | 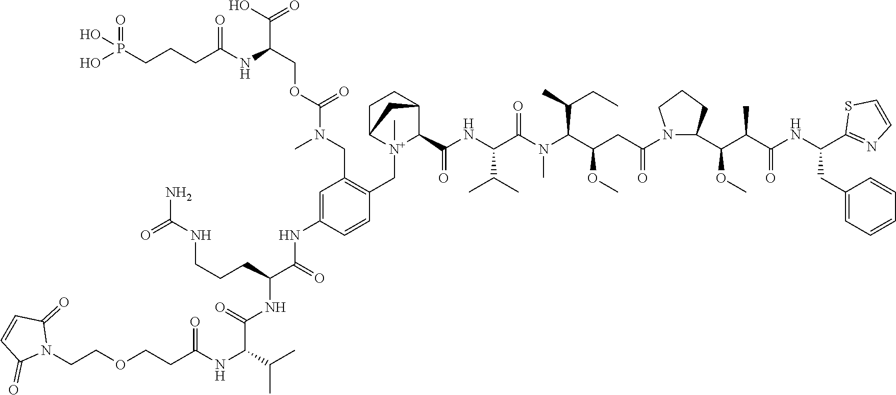 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)OC[C@@H](NC(=O)CCCP(=O)(O)O)C(=O)O)C(C)C] |
| L206- P2 | 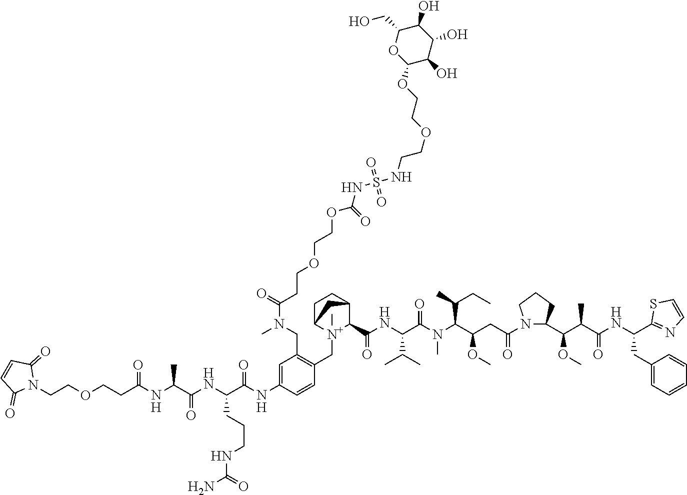 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@H](C)NC(=O)CCOCCN2C(=O)C=CC2=O)cc1CN(C)C(=O)CCOCCOC(=O)NS(=O)(=O)NCCOCCO[C@@H]1O[C@H](CO)[C@@H](O)[C@H](O)[C@H]1O)C(C)C] |
| L207- P2 | 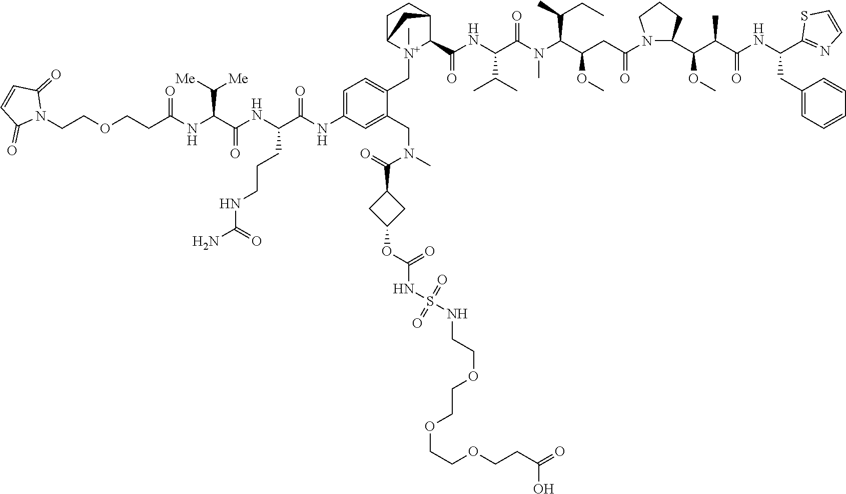 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCOCCC(=O)O)C1)C(C)C] |
| L208- P2 | 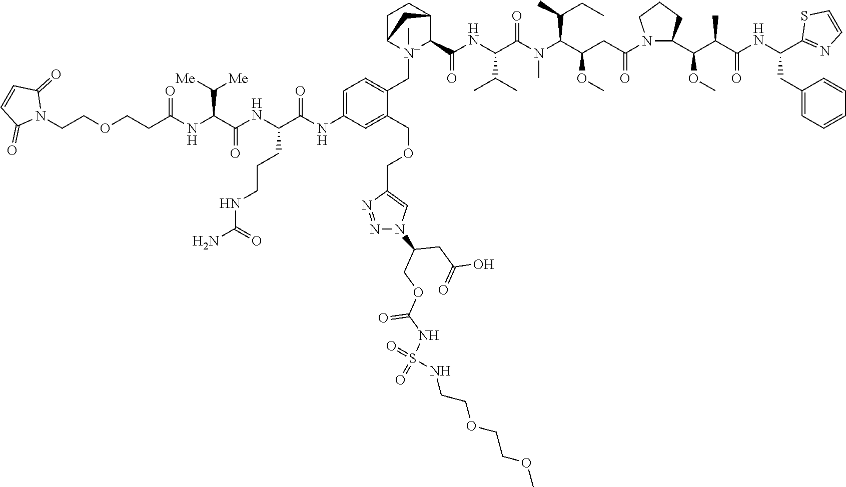 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)[N+]1(C)Cc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)NS(=O)(=O)NCCOCCOC)CC(=O)O)nn1)C(C)C] |

### Example 4: Synthesis of Non-Pegylated Linker-Drug Compounds

### Example 4-1: Synthesis of 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L1-P1)

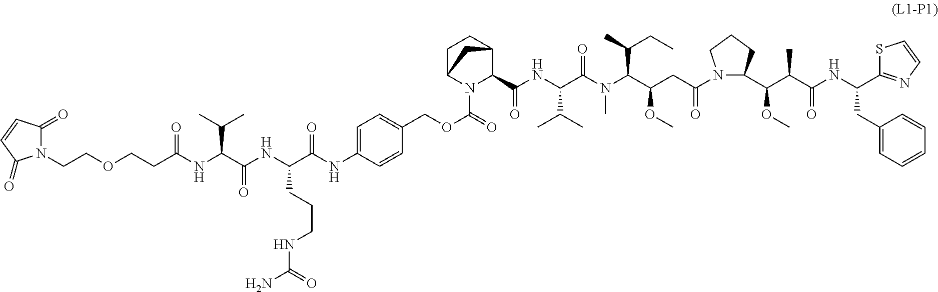 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1)C(C)C]

### Step 1. Synthesis of 4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

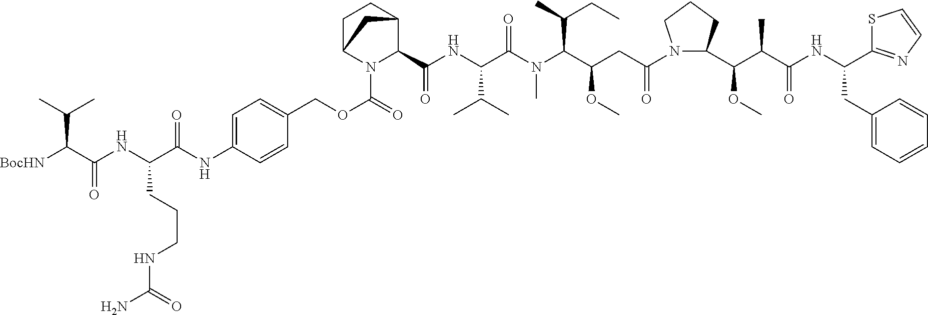 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)OC(C)(C)C)C(C)C)cc1)C(C)C]

4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained using the procedure described in Example 3-1, step 2, however tert-butyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate was replaced with tert-butyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (36 mg, 0.056 mmoles, 1.0 equiv.). 4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: (65 mg, 0.051 mmoles, 91%). LCMS: MH+=781.4 (fragment), Rt=2.55 min (5 min acidic method-Method C).

### Step 2. Synthesis of 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L1-P1)

4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L1-P1) was obtained using the procedure described in Example 3-1, step 4, however 2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was replaced with 4-((S)-2-((S)-2-((tert-butoxycarbonyl)amino)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (25.5 mg, 0.020 mmoles, 1.0 equiv.). 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (L1-P1): (19 mg, 0.014 mmoles, 68%). HRMS: M+=1381.7100, Rt=2.52 min (5 min acidic method-Method D).

### Example 5: Generation and Characterization of P-Cadherin Antibody Drug Conjugates

### Example 5A: Preparation of P-Cadherin Antibody with Specific Cysteine (Cys) Mutations

Preparation of anti-p-Cadherin antibodies with site-specific cysteine mutations, in particular P-Cad mab2, has been described previously in WO 2016/203432 (as NOV169N31Q), the disclosure of which is herein incorporated by reference.

### Reduction, Reoxidation and Conjugation of Cys Mutant Anti-p-Cadherin Antibodies to Liner-Drugs of the Invention

Because engineered Cys residues in antibodies expressed in mammalian cells are modified by adducts (disulfides) such as glutathione (GSH) and/or cysteine during biosynthesis (Chen et al. 2009), the modified Cys as initially expressed is unreactive to thiol reactive reagents such as maleimido or bromo-acetamide or iodo-acetamide groups. To conjugate engineered Cys residues, glutathione or cysteine, adducts need to be removed by reducing disulfides, which generally entails reducing all disulfides in the expressed antibody. This can be accomplished by first exposing antibody to a reducing agent such as dithiothreitol (DTT) followed by reoxidation of all native disulfide bonds of the antibody to restore and/or stabilize the functional antibody structure. Accordingly, in order to reduce native disulfide bonds and disulfide bonds between the cysteine or GSH adducts of engineered Cys residue(s), freshly prepared DTT was added to previously purified Cys mutant antibodies to a final concentration of 10 mM or 20 mM. After antibody incubation with DTT at 37° C. for 1 hour, mixtures were dialyzed against PBS for three days with daily buffer exchange to remove DTT. Alternatively, DTT can be removed by a gel filtration step. After removal of DTT, antibody solutions are allowed to reoxidize to reform native disulfide bonds. The reoxidation process was monitored by reverse-phase HPLC, which is able to separate antibody tetramer from individual heavy and light chain molecules. Reactions were analyzed on a PRLP-S 4000A column (50 mm×2.1 mm, Agilent) heated to 80° C. and column elution was carried out by a linear gradient of 30-60% acetonitrile in water containing 0.1% TFA at a flow rate of 1.5 ml/min. The elution of proteins from the column was monitored at 280 nm. Incubation was allowed to continue until reoxidation was complete. After reoxidation, a maleimide-containing compound (either (L1-P1), (L2-P1), (L3-P1), (L4-P1) or (L5-P1) or (L6-P1)) was added to reoxidized antibodies in PBS buffer (pH 7.2) at molar ratios of typically 1:1, 1.5:1, 2.5:1, or 5:1 to engineered Cys, and incubations were carried out for 5 to 60 minutes or longer. Typically, excess free compound was removed by purification over Protein A resin by standard methods followed by buffer exchange into PBS.

Cys mutant antibodies were alternatively reduced and reoxidized using an on-resin method. Protein A Sepharose beads (1 ml per 10 mg antibody) were equilibrated in PBS (no calcium or magnesium salts) and then added to an antibody sample in batch mode and incubated for 15-20 minutes. A stock of 0.5 M cysteine was prepared by dissolving 850 mg of cysteine HCl in 10 ml of a solution prepared by adding 3.4 g of NaOH to 250 ml of 0.5 M sodium phosphate pH 8.0. 20 mM cysteine was then added to the antibody/bead slurry, and mixed gently at room temperature for 30-60 minutes. Beads were loaded to a gravity column and washed with 50 bed volumes of PBS in less than 30 minutes, then the column was capped with beads resuspended in one bed volume of PBS. To modulate the rate of reoxidation, 50 nM to 1 M copper chloride was optionally added. The reoxidation progress was monitored at various time points where 25 μL of resin slurry was removed, 1 μL of 20 mM MC-valcit-MMAE was added, and the tube agitated several times. The resin was then centrifuged, supernatant removed, and then eluted with 50 μL Antibody elution buffer (Thermo) and the resin pelleted with the supernatant analyzed by reverse phase chromatography using an Agilent PLRP-S 4000A 5 μm, 4.6×50 mm column (Buffer A is water, 0.1% TFA, Buffer B Acetonitrile, 0.1% TFA, column held at 80° C., Flowrate 1.5 ml/min). Once reoxidation progressed to desired completeness, conjugation could be initiated immediately by addition of 1-5 molar equivalent of compound (either (L1-P1), (L2-P1), (L3-P1), (L4-P1) or (L5-P1) or (L6-P1)) over engineered cysteines, and allowing the mixture to react for 5-10 minutes at room temperature before the column was washed with at least 20 column volumes of PBS. Antibody conjugates were eluted with Antibody elution buffer (Thermo) and neutralized with 0.1 volumes 0.5 M sodium phosphate pH 8.0 and buffer exchanged to PBS.

Alternatively, instead of initiating conjugation with antibody on the resin, the column was washed with at least 20 column volumes of PBS, and antibody was eluted with IgG elution buffer and neutralized with buffer pH 8.0. Antibodies were then either used for conjugation reactions or flash frozen for future use.

### Example 5B: (P-Cad mab2-L1-P1)

To a solution of P-Cad mab2 antibody (4.0 mg, 800 μL of a 5.0 mg/mL solution in 1×PBS buffer solution, 0.027 μmoles, 1.0 equiv.) was added L1-P1 (10.76 μL of a 20 mM solution in DMSO, 0.215 μmoles, 8.0 equiv.). The resulting mixture was shaken at 400 rpm at ambient temperature for 1 hour, at which time the mixture was purified by ultracentrifugation (4 mL Amicon 10 kD cutoff membrane filter, diluting sample to 4 mL total volume with PBS buffer followed by centrifugation for 10 minutes at 7500×g, repeated 6 times). After dilution to 5.0 mg/mL, conjugate P-Cad mab2-L1-P1 was obtained (4.08 mg, 0.027 μmoles, 99%). HRMS data (protein method) indicates a mass of 154192, with a DAR of 3.8 as calculated by comparing MS intensities of peaks for DAR3 and DAR4 species. Size-exclusion chromatography (SEC) indicates <1% aggregation, as determined by comparison of the area of the high-molecular-weight peak absorbance at 210 and 280 nm with the area of the peak absorbance for monomeric ADC.

### Example 5C: (P-Cad mab2-L4-P1)

Following the procedure described in Example 5B using P-Cad mab2 antibody (2.5 mg, 500 μL of a 5.0 mg/mL solution, 0.017 μmoles, 1.0 equiv.) and L4-P1 (13.45 μL of a 10 mM solution in DMSO, 0.135 μmoles, 8.0 equiv.), conjugate P-Cad mab2-L4-P1 was obtained (2.64 mg, 0.017 μmoles, 99%). HRMS data (protein method) indicates a mass of 156104, with a DAR of 3.9. SEC indicates <1% aggregation.

### Example 5D: (P-Cad mab2-L2-P1)

Following the procedure described in Example 5B using P-Cad mab2 antibody (2.0 mg, 400 μL of a 5.0 mg/mL solution, 0.027 μmoles, 1.0 equiv.) and L2-P1 (5.38 μL of a 20 mM solution in DMSO, 0.108 μmoles, 8.0 equiv.), conjugate P-Cad mab2-L2-P1 was obtained (2.01 mg, 0.013 μmoles, 96%). HRMS data (protein method) indicates a mass of 156333, with a DAR of 3.9. SEC indicates <1% aggregation.

### Example 5E: (P-Cad mab2-L3-P1)

Following the procedure described in Example 5B using P-Cad mab2 antibody (2.5 mg, 500 μL of a 5.0 mg/mL solution, 0.017 μmoles, 1.0 equiv.) and L3-P1 (5.89 μL of a 20 mM solution in DMSO, 0.118 μmoles, 7.0 equiv.), conjugate P-Cad mab2-L3-P1 was obtained (2.15 mg, 0.014 μmoles, 81%). HRMS data (protein method) indicates a mass of 158065, with a DAR of 4.0. SEC indicates 1% aggregation.

### Example 5F: (P-Cad mab2-L5-P1)

Following the procedure described in Example 5B using P-Cad mab2 antibody (2.5 mg, 500 μL of a 5.0 mg/mL solution, 0.017 μmoles, 1.0 equiv.) and L5-P1 (5.89 μL of a 20 mM solution in DMSO, 0.118 μmoles, 7.0 equiv.), conjugate P-Cad mab2-L5-P1 was obtained (2.31 mg, 0.015 μmoles, 88%). HRMS data (protein method) indicates a mass of 156446, with a DAR of 3.7. SEC indicates 1% aggregation.

### Example 5G: (P-Cad mab2-L6-P1)

Following the procedure described in Example 5B using P-Cad mab2 antibody (2.5 mg, 500 μL of a 5.0 mg/mL solution, 0.017 μmoles, 1.0 equiv.) and L6-P1 (13.44 μL of a 10 mM solution in DMSO, 0.134 μmoles, 8.0 equiv.), conjugate P-Cad mab2-L6-P1 was obtained (2.28 mg, 0.015 μmoles, 86%). HRMS data (protein method) indicates a mass of 158100, with a DAR of 3.8. SEC indicates <1% aggregation.

### Example 6: In Vitro Evaluation of Anti-P-Cadherin ADCs

### Cell Lines

The antibody drug conjugates were tested against four endogenous cancer cell lines and one isogenic cell line engineered to overexpress the target of interest. FaDu (ATCC No. HTB-43 cultured in Eagle's Minimum Essential Medium+10% FBS), HCC70 (ATCC No. CRL-2315 cultured in RPMI-1640+10% FBS), HCC1954 (ATCC No. CRL-2338 cultured in RPMI-1640+10% FBS) and HT-29 (ATCC No. HTB-38 cultured in McCoy's 5a Medium Modified+10% FBS). The HT-29 cell line was transfected to generate a stable HT-29 cell line expressing the exogenous protein of interest, P-cadherin, HT-29 PCAD+(cultured in McCoy's 5a Medium Modified+10% FBS).

### Inhibition of Cell Proliferation and Survival

The ability of the P-cadherin linker variant antibody drug conjugates to inhibit cell proliferation and survival was assessed using the Promega CellTiter-Glo® proliferation assay.

The cell lines were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, cells were lifted off tissue culture flasks using 0.25% trypsin. Cell viability and cell density were determined using a cell counter (Vi-Cell XR Cell Viability Analyzer, Beckman Coulter). Cells with higher than 85% viability were seeded in white clear bottom 384-well TC treated plates (Corning cat. #3765). HT-29 cells and HT-29 PCAD+ cells were seeded at a density of 500 cells per well in 45 μL of standard growth media. FaDu, HCC70 and HCC1954 cells were seeded at a density of 1,500 cells per well in 45 μL of standard growth media. Plates were incubated at 5% CO₂, 37° C. overnight in a tissue culture incubator. The next day, free MMAE (monomethyl auristatin E), P-cadherin targeting ADCs and non-targeting isotype ADCs were prepared at 10× in standard growth media. The prepared drug treatments were then added to the cells resulting in final concentrations of 0.0076-150 nM and a final volume of 50 μL per well. Each drug concentration was tested in quadruplets. Plates were incubated at 5% CO₂, 37° C. for 5 days in a tissue culture incubator, after which cell viability was assessed through the addition of 25 μL of CellTiter Glo® (Promega, cat #G7573), a reagent which lyses cells and measures total adenosine triphosphate (ATP) content. Plates were incubated at room temperature for 10 minutes to stabilize luminescent signals prior to reading using a luminescence reader (EnVision Multilabel Plate Reader, PerkinElmer). To evaluate the effect of the drug treatments, luminescent counts from wells containing untreated cells (100% viability) were used to normalize treated samples. A variable slope model was used to fit a nonlinear regression curve to the data in GraphPad PRISM version 7.02 software. IC50 and Amax values were extrapolated from the resultant curves.

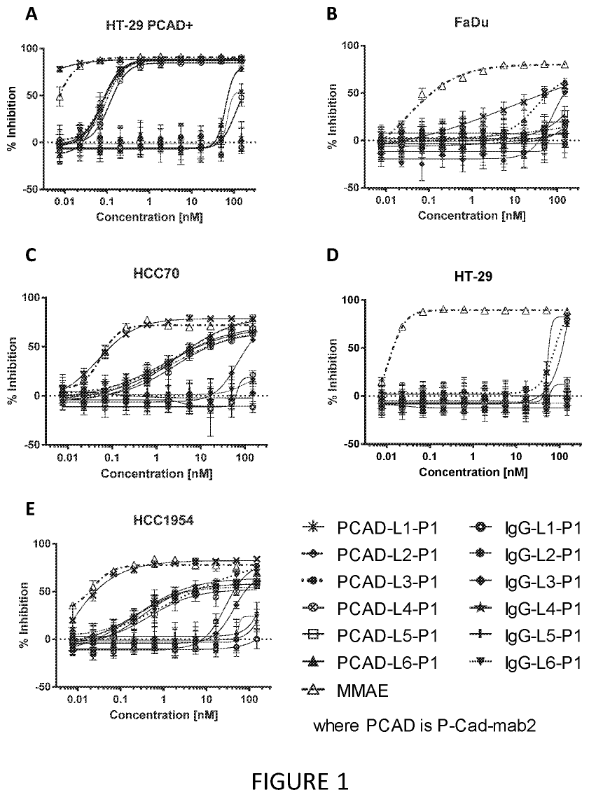

The dose response curves of five representative cancer cell lines are shown in FIG. 1. The concentrations of treatment required to inhibit 50% of cell growth or survival (IC₅₀) were calculated with representative IC₅₀ values of the cell lines tested summarized in Table 5.

TABLE 5
Antibody Drug Conjugate activity in a panel of human cancer cell lines. IC50 (nM) values of P-cadherin targeting
ADCs in comparison with free maytansine and isotype control ADCs in the panel of cell lines. The values
reported here are values from individual assays which are representative of multiple replicates.

| | HT-29 | | HT-29 PCAD+ | | FaDu | | HCC-70 | | HCC-1954 | |
|---|---|---|---|---|---|---|---|---|---|---|
| Cell Line | IC50 | | IC50 | | IC50 | | IC50 | | IC50 | |
| Analysis | (nM) | Amax | (nM) | Amax | (nM) | Amax | (nM) | Amax | (nM) | Amax |
| PCAD-mab2- | — | — | 0.077 | 90.03 | — | — | 0.237 | 65.09 | 1.598 | 74.97 |
| L1-P1 | | | | | | | | | | |
| PCAD-mab2- | — | — | 0.098 | 83.91 | — | — | 0.336 | 51.05 | 2.313 | 72.21 |
| L2-P1 | | | | | | | | | | |
| PCAD-mab2- | 78.660 | 93.55 | 0.083 | 86.77 | 32.900 | 68.04 | 1.352 | 105.30 | 3.733 | 85.02 |
| L3-P1 | | | | | | | | | | |
| PCAD-mab2- | — | — | 0.117 | 86.16 | — | — | 0.467 | 57.16 | 2.581 | 69.95 |
| L4-P1 | | | | | | | | | | |
| PCAD-mab2- | — | — | 0.075 | 91.61 | — | — | 0.320 | 65.88 | 1.879 | 75.72 |
| L5-P1 | | | | | | | | | | |
| PCAD-mab2- | 0.030 | 3.64 | 0.069 | 101.60 | — | — | 0.209 | 80.22 | 2.626 | 87.77 |
| L6-P1 | | | | | | | | | | |
| PCAD-mab2- | — | — | — | — | 8.923 | 104.90 | 0.004 | 170.80 | 0.048 | 83.98 |
| L7-P2 | | | | | | | | | | |
| IgG-L1-P1 | — | — | — | — | 0.013 | 0.30 | — | — | 3.739 | 6.43 |
| IgG-L2-P1 | — | — | — | — | — | — | — | — | — | — |
| IgG-L3-P1 | — | — | 62.87 | 86.12 | 76.500 | 90.57 | 43.930 | 72.36 | 69.250 | 80.62 |
| IgG-L4-P1 | — | — | — | — | 7.398 | 1.38 | — | — | 0.108 | 7.17 |
| IgG-L5-P1 | — | — | — | — | — | — | — | — | — | — |
| IgG-L6-P1 | 4.274 | 2.66 | — | — | — | — | — | — | 80.760 | 17.80 |
| IgG-L7-P2 | — | — | 105.00 | 72.83 | — | — | 27.920 | 89.52 | — | — |
| Free MMAE | 0.012 | 102.30 | — | — | 0.032 | 108.80 | 0.022 | 49.22 | 0.056 | 65.26 |

### Induction of Caspase-3/7 Activity

In addition to the impact on proliferation, P-cadherin targeting ADCs with linker variants were also evaluated for their ability to induce Caspase-3/7 activity.

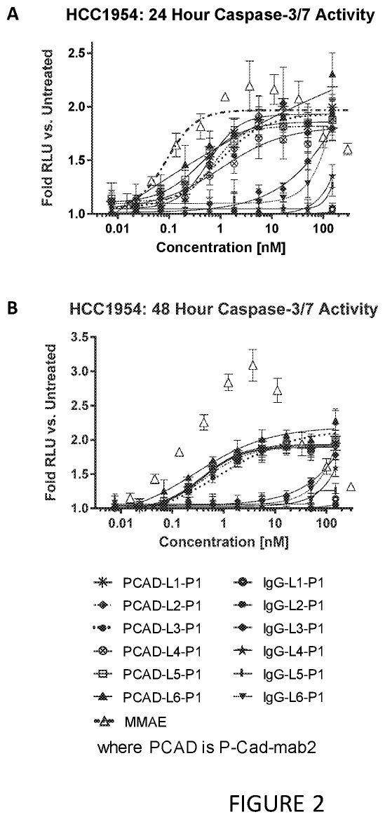

The cell line, HCC1954, was cultured in media that is optimal for growth at 5% CO₂, 37° C. in a tissue culture incubator. Prior to seeding for the assay, the cells were split at least 2 days before the assay to ensure optimal growth density. On the day of seeding, cells were lifted off tissue culture flasks using 0.25% trypsin. Cell viability and cell density were determined using a cell counter (Vi-Cell XR Cell Viability Analyzer, Beckman Coulter). Cells with higher than 85% viability were seeded in white clear bottom 384-well TC treated plates (Corning cat. #3765) at a density of 3,000 cells per well in 20 μL of standard growth media. Plates were incubated at 5% CO₂, 37° C. overnight in a tissue culture incubator. The next day, free MMAE (monomethyl auristatin E), P-cadherin targeting ADCs and non-targeting isotype ADCs were prepared at 5× in standard growth media. The prepared drug treatments were then added to the cells resulting in final concentrations of 0.0076-300 nM and a final volume of 25 μL per well. Each drug concentration was tested in quadruplets. Plates were incubated at 5% CO₂, 37° C. for 24 and 48 hours in a tissue culture incubator, after which caspase-3/7 activity was assessed through the addition of 25 μL of Caspase-Glo® 3/7 (Promega, cat #G8093), a reagent which lyses cells and generates a luminescent signal following caspase cleavage of a luminogenic caspase-3/7 substrate. The plates were incubated in the dark at room temperature on an orbital shaker at a speed that provides adequate mixing for 5 minutes to induce cell lysis. Plates were then incubated at room temperature for 30 minutes to stabilize luminescent signals prior to reading using a luminescence reader (EnVision Multilabel Plate Reader, PerkinElmer). To evaluate the effect of the drug treatments, luminescent counts from wells containing untreated cells (100% viability) were used to normalize treated samples. A variable slope model was used to fit a nonlinear regression curve to the data in GraphPad PRISM version 7.02 software. The dose response curves of HCC1954 are shown in FIG. 2.

### Example 7: In Vivo Efficacy of Anti-P-Cadherin ADCs Against the HCC70 Triple Negative Breast Cancer (TNBC) Model in Mice

As the above in vitro studies had shown target-dependent and potent inhibition of cell growth in the HCC70 cell line by anti-PCAD-ADCs the antitumor activity of these ADCs in vivo in this TNBC model was evaluated.

### Methods

HCC70 cells were cultured at 37° C. in an atmosphere of 5% CO₂ in air in RPMI1640 (BioConcept Ltd. Amimed) supplemented with 10% FCS (BioConcept Ltd. Amimed, #2-01F30), 2 mM L-glutamine (BioConcept Ltd. Amimed, #5-10K00-H), 1 mM sodium pyruvate (BioConcept Ltd. Amimed, #5-60F00-H), 10 mM HEPES (Gibco #11560496) and 14 mM D-glucose (Life Technologies, #A2494001). To establish HCC70 xenografts cells were harvested and re-suspended in HBSS (Gibco, #14175) and mixed with Matrigel (BD Bioscience, #354234) (1:1 v/v) before injecting 100 μL containing 1×10⁷ cells subcutaneously close to a mammary fat pad of female SCID beige mice (Charles River, Germany). Tumor growth was monitored regularly post cell inoculation and animals were randomised into treatment groups (n=6) with a mean tumor volume of about 200 mm³. A control group was untreated, and the rest of the groups were treated with isotype-ADC or anti-PCAD-ADCs by administering a single intravenous (iv) dose of 5 mg/kg. A 5 mg/kg dose was selected as it was expected to provide a window to discern differences among ADC candidates in this model. Doses were adjusted to individual mouse body weights. The iv dose volume was 10 ml/kg and each ADC, dissolved in 0.9% (w/v) NaCl in water.
Tumor volume data on day 20 post treatment were analyzed statistically using GraphPad Prism 7.00 (GraphPad Software). The tumor volumes were extrapolated to day 20 if they were measured on days on either side of day 20. If the variances in the data were normally distributed, the data were analyzed using one-way ANOVA with post hoc Dunnett's test for comparison of treatment versus untreated control group. For the comparison of tumor volume of the isotype control group versus the corresponding ADC treated group t-test was used when data were normally distributed or Mann Whitney test was when the data were not normally distributed. When applicable, results are presented as mean±SEM.
As a measure of efficacy the % T/C value is calculated at the end of the experiment according to:

$${( Δtumor {volume}^{treated} \text{/} Δtumor {volume}^{control} )}^{*} 100$$

Tumor regression was calculated according to:

$$- {( Δtumor {volume}^{treated} \text{/} tumor {volume}^{treated at start} )}^{*} 100$$

Where Δtumor volumes represent the mean tumor volume on the evaluation day minus the mean tumor volume at the start of the experiment.

### Results: Efficacy and Tolerability

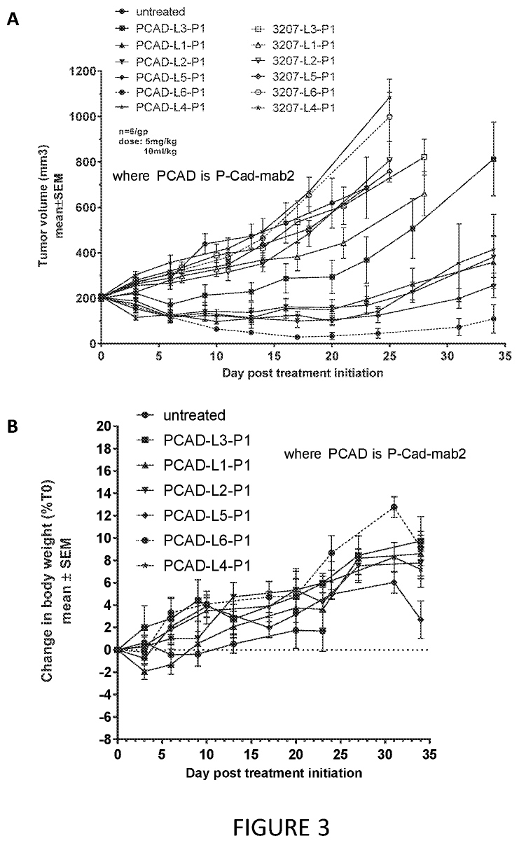

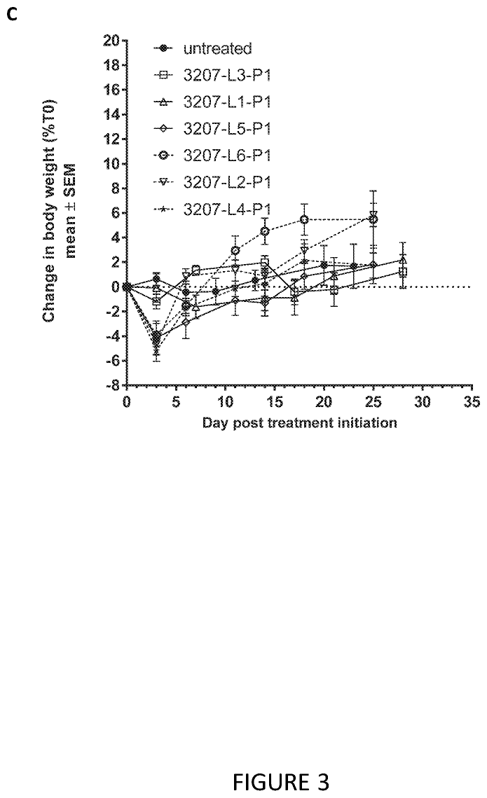

The mean tumor volumes in all anti-PCAD-ADC treated groups were significantly different from the untreated group and from their corresponding hulgG1 isotype-matched ADC control group on day 20 (One way ANOVA; Dunn's Method, or t-test or Mann Whitney test, p<0.05) (Table 6, FIG. 3). No significant body weight loss was observed in any group on day 20 compared to the untreated group (FIG. 3).

TABLE 6
Summary of the antitumor effect and tolerability of anti-PCAD-ADCs
and the hulgG1 isotype-matched control ADCs in the HCC70 human
TNBC xenograft model in SCID-beige female mice. Delta tumor volumes
were calculated for day 20 and are presented as means ± SEM.

| | | Tumor response | | | Survival |
|---|---|---|---|---|---|
| | Dose, | | | ΔTumor | (alive/total); On |
| | schedule, | | Regression | volume | treatment day |
| Test agent | route | T/C (%) | (%) | (mm³)^{$} | 20 |
| Untreated | — | 100 | — | 486 | 6/6 |
| 3207-L1-P1 | 5.0 mg/kg, | 56 | | 233 | 6/6 |
| | Day 0, i.v | | | | |
| PCAD-mab2- | 5.0 mg/kg, | | 25*^{$} | −51 | 6/6 |
| L1-P1 | Day 0, i.v | | | | |
| 3207-L2-P1 | 5.0 mg/kg, | 88 | | 368 | 6/6 |
| | Day 0, i.v | | | | |
| PCAD-mab2- | 5.0 mg/kg, | | 21*^{$} | −43 | 6/6 |
| L2-P1 | Day 0, i.v | | | | |
| 3207- L3-P1 | 5.0 mg/kg, | 92 | — | 386 | 6/6 |
| | Day 0, i.v | | | | |
| PCAD-mab2- | 5.0 mg/kg, | 22*^{$} | | 93 | 6/6 |
| L3-P1 | Day 0, i.v | | | | |
| 3207-L4-P1 | 5.0 mg/kg, | 139 | | 582 | 6/6 |
| | Day 0, i.v | | | | |
| PCAD-mab2- | 5.0 mg/kg, | | 52*^{$} | −108 | 6/6 |
| L4-P1 | Day 0, i.v | | | | |
| 3207-L5-P1 | 5.0 mg/kg, | 89 | — | 372 | 6/6 |
| | Day 0, i.v | | | | |
| PCAD-mab2- | 5.0 mg/kg, | | 49*^{$} | −101 | 6/6 |
| L5-P1 | Day 0, i.v | | | | |
| 3207-L6-P1 | 5.0 mg/kg, | 130 | — | 544 | 6/6 |
| | Day 0, i.v | | | | |
| PCAD-mab2- | 5.0 mg/kg, | | 84*^{$} | −173 | 6/6 |
| L6-P1 | Day 0, i.v | | | | |

*p < 0.05, compared to untreated control, one way ANOVA with post hoc Dunnett's test;
^{$}p < 0.05, compared with corresponding isotype control (t-test or Mann Whitney test).

It is understood that the examples and embodiments described herein are for illustrative purposes only and that various modifications or changes in light thereof will be suggested to persons skilled in the art and are to be included within the spirit and purview of this application and the scope of the appended claims.

1. A compound of Formula (I), or pharmaceutically acceptable salt thereof: 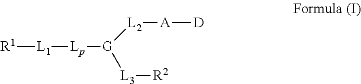
wherein:
R¹ is a reactive group;
L₁ is a bridging spacer;
Lp is a bivalent peptide spacer;
G-L₂-A is a self-immolative spacer;
R² is a hydrophilic moiety;
L₂ is a bond, a methylene, a neopentylene or a C₂-C₃alkenylene;
A is a bond, —OC(═O)—*, 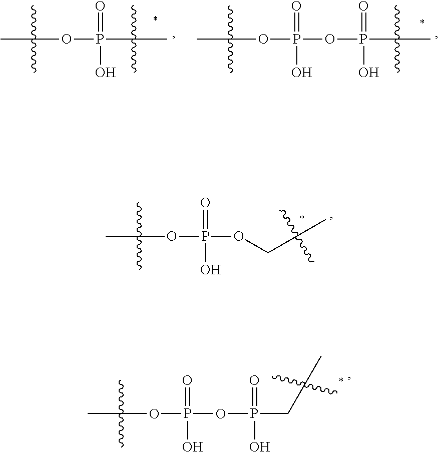 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
and
D is a Drug moiety comprising an N or an O, wherein D is connected to A via a direct bond from A to the N or the O of the Drug moiety.

2. The compound of Formula (I), or pharmaceutically acceptable salt thereof, of claim 1 wherein:
R¹ is a reactive group;
L₁ is a bridging spacer;
Lp is a bivalent peptide spacer comprising one to four amino acid residues;
the 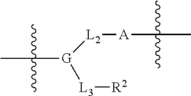
group is selected from: 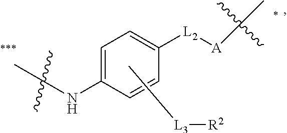
wherein the * of 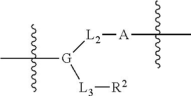
indicates the point of attachment to an N or a O of the Drug moiety, the *** of 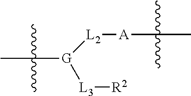
indicates the point of attachment to Lp;
R² is a hydrophilic moiety;
L₂ is a bond, a methylene, a neopentylene or a C₂-C₃alkenylene;
A is a bond, —OC(═O)—*, 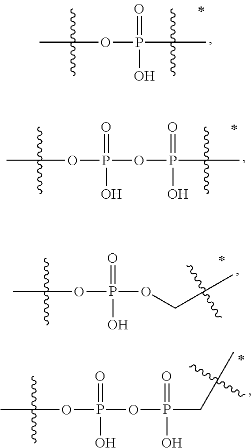 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
and
D is a Drug moiety comprising an N or an O, wherein D is connected to A via a direct bond from A to the N or the O of the Drug moiety.

3. The compound of Formula (II), or pharmaceutically acceptable salt thereof, is a compound having a structure of Formula (II), or pharmaceutically acceptable salt thereof, 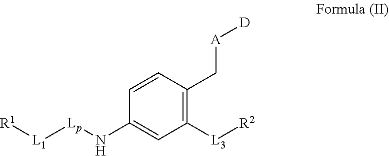
wherein:
R¹ is a reactive group;
L₁ is a bridging spacer;
Lp is a bivalent peptide spacer comprising one to four amino acid residues;
R² is a hydrophilic moiety;
A is a bond, —OC(═O)—*, 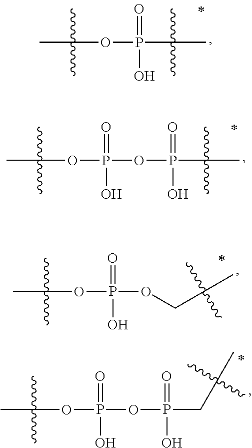 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
and
D is a Drug moiety comprising an N or an O, wherein D is connected to A via a direct bond from A to the N or the O of the Drug moiety.

4. The compound of claim 1, or pharmaceutically acceptable salt thereof, wherein:
R¹ is 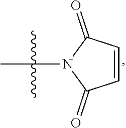 [SMILES: *N1C(=O)C=CC1=O]
—ONH₂, —NH₂, 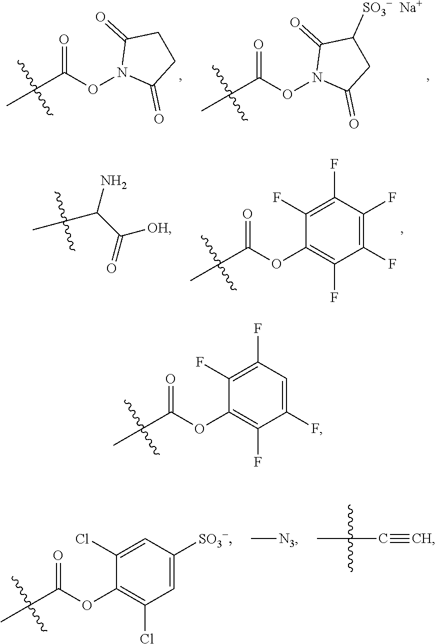
—SH, —SR³, —SSR⁴, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, 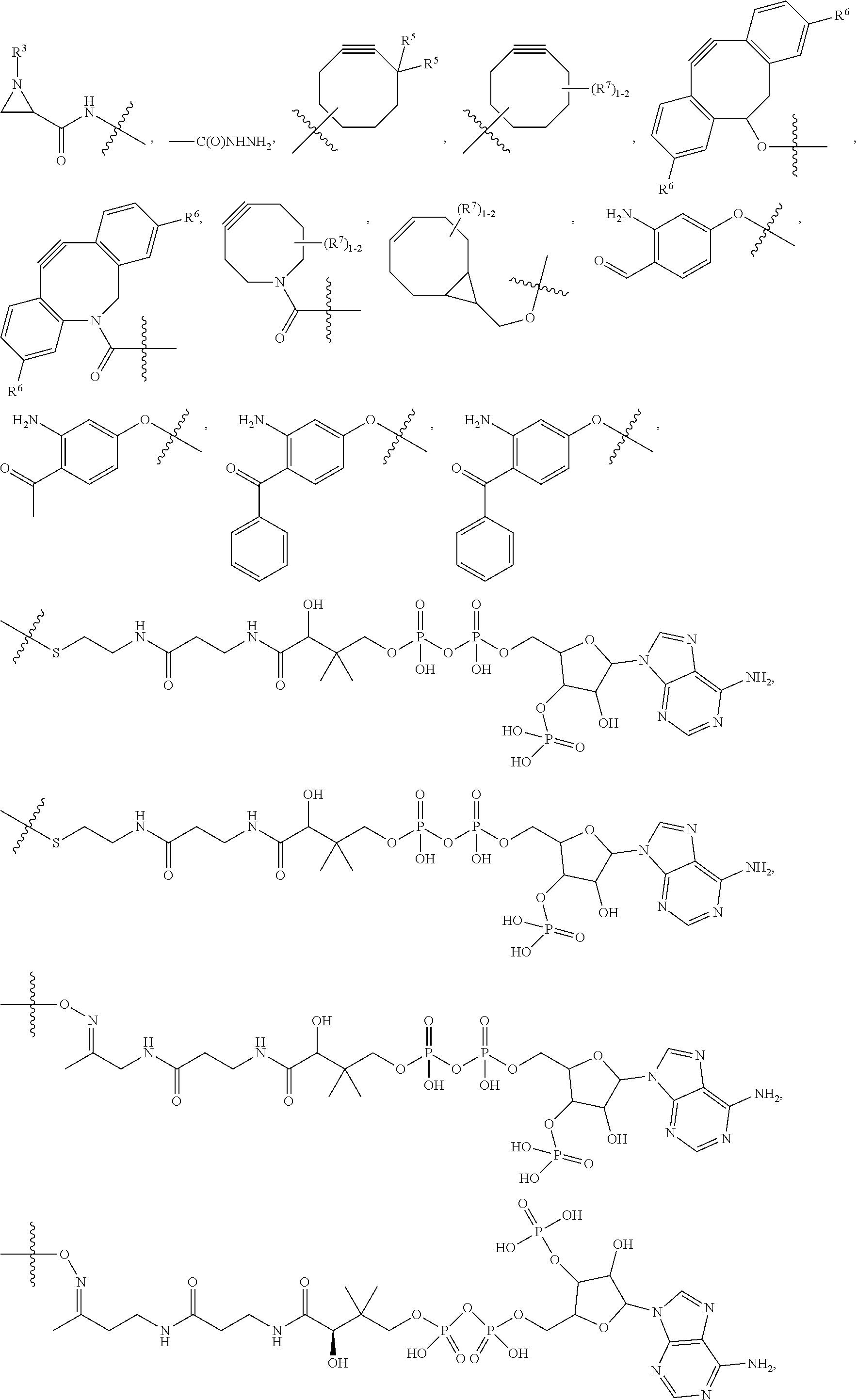 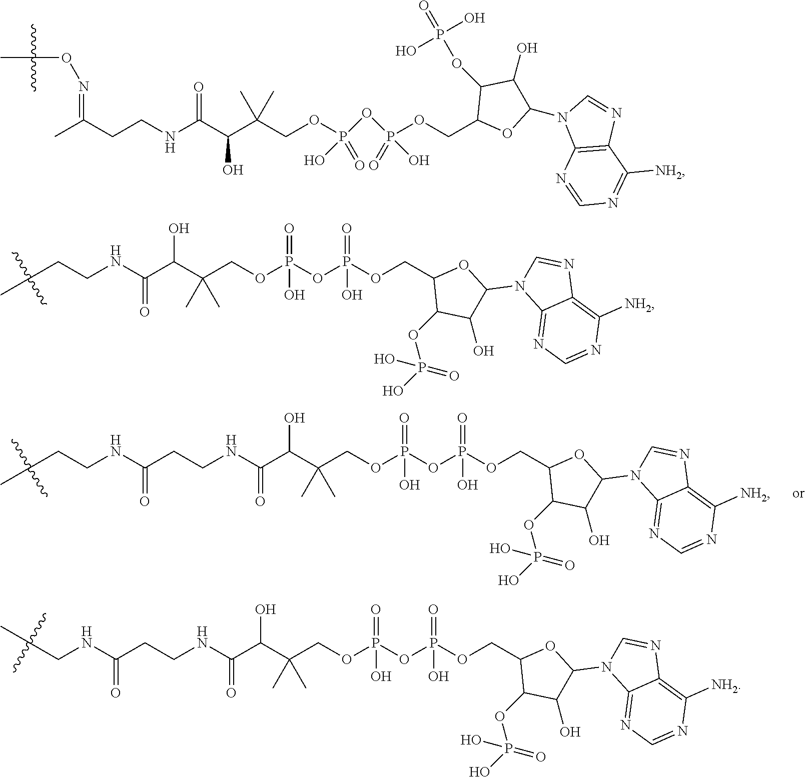
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp, and the ** of L₁ indicates the point of attachment to R¹;
R² is a hydrophilic moiety selected from polyethylene glycol, polyalkylene glycol, a sugar, an oligosaccharide, a polypeptide, a polysarcosine, or C2-C₆alkyl substituted with 1 to 3 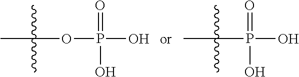 [SMILES: *OP(=O)(O)O ; *P(=O)(O)O]
groups;
each R³ is independently selected from H and C₁-C₆alkyl;
R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —CN, —NO₂ and —OH;
each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
X₁ is 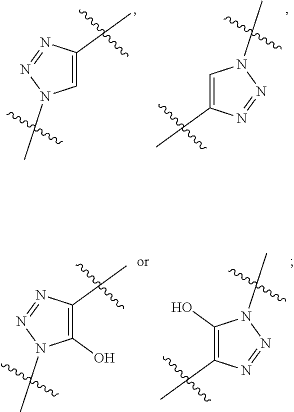 [SMILES: *c1cn(*)nn1 ; *c1cn(*)nn1 ; *c1nnn(*)c1O ; *c1nnn(*)c1O]
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising one to four amino acid residues independently selected from glycine, valine, citrulline, lysine, isoleucine, phenylalanine, methionine, asparagine, proline, alanine, leucine, tryptophan, and tyrosine;
A is a bond, —OC(═O)—*, 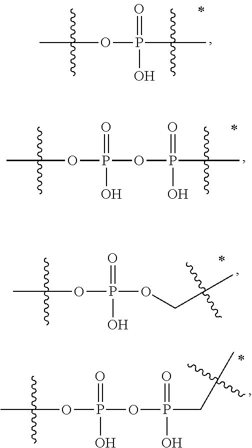 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety having the structure 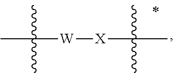
where
(i) W is —CH₂O—**, —CH₂N(R^{b})C(═O)O—**, —NHC(═O)C(R^{b})₂NHC(═O)O—**, —NHC(═O)C(R^{b})₂NH—**, —NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—**, —NH—, or —CH₂N(R^{b})C(═O)CH₂—**, wherein each R^{b} is independently selected from H, C₁-C₆alkyl or C₃-C₈cycloalkyl and wherein the ** of W indicates the point of attachment to X;
X is a bond, triazolyl or ***—CH₂-triazolyl*, wherein the *** of X indicates the point of attachment to W and the * of X indicates the point of attachment to R²; or
(ii) W is —CH₂O—**, —CH₂N(R^{b})C(═O)O—**, —NHC(═O)C(R^{b})₂NHC(═O)O—**, —NHC(═O)C(R^{b})₂NH—**, NHC(═O)C(R^{b})₂NHC(═O)—**, —CH₂N(X—R²)C(═O)O—**, —C(═O)N(X—R²)—**, —CH₂N(X—R²)C(═O)—** —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
X is ***—CH₂-triazolyl-C₁₋₄ alkylene-OC(O)NHS(O)₂NH—*, ***—C₄₋₆ cycloalkylene-OC(O)NHS(O)₂NH—*, ***—(CH₂CH₂O)ₙ—C(O)NHS(O)₂NH—*, ***—(CH₂CH₂O)ₙ—C(O)NHS(O)₂NH—(CH₂CH₂O)ₙ—*, or ***—CH₂-triazolyl-C₁₋₄ alkylene-OC(O)NHS(O)₂NH—(CH₂CH₂O)ₙ—*, wherein each n independently is 1, 2, or 3, the *** of X indicates the point of attachment to W and the * of X indicates the point of attachment to R²;
and
the * of L₃ indicates the point of attachment to R²;
and
D is a Drug moiety comprising an N or an O, wherein D is connected to A via a direct bond from A to the N or the O of the Drug moiety.

5. The compound of claim 1, or pharmaceutically acceptable salt thereof, wherein:
R₁ is 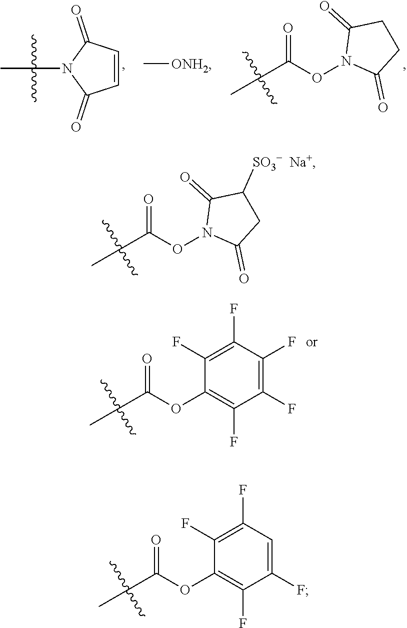
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 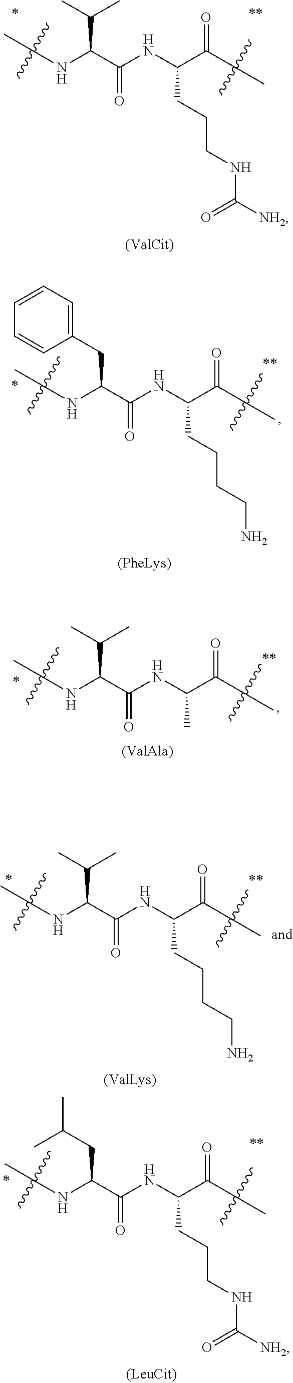 [SMILES: *N[C@H](C(=O)N[C@@H](CCCNC(N)=O)C(*)=O)C(C)C ; *N[C@@H](Cc1ccccc1)C(=O)N[C@@H](CCCCN)C(*)=O ; *N[C@H](C(=O)N[C@@H](C)C(*)=O)C(C)C ; *N[C@H](C(=O)N[C@@H](CCCCN)C(*)=O)C(C)C ; *N[C@@H](CC(C)C)C(=O)N[C@@H](CCCNC(N)=O)C(*)=O]
where the * of Lp indicates the attachment point to L₁ and the ** of Lp indicates the attachment point to the —NH— group of G;
L₃ is a spacer moiety having the structure 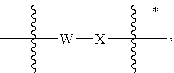
where
W is —CH₂O—**, —CH₂N(R^{b})C(═O)O—**, —NHC(═O)CH₂NHC(═O)O—** —CH₂N(X—R²)C(═O)O—**, —C(═O)N(X—R²)—**, —CH₂N(X—R²)C(═O)—** —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
X is a bond, triazolyl or ***—CH₂-triazolyl*, wherein the *** of X indicates the point of attachment to W and the * of X indicates the point of attachment to R²;
and
the * of L₃ indicates the point of attachment to R²;
R² is a hydrophilic moiety selected from polyethylene glycol, polyalkylene glycol, a sugar, an oligosaccharide, a polysarcosine, a polypeptide or C₂-C₆alkyl substituted with 1 to 3 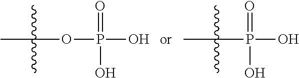 [SMILES: *OP(=O)(O)O ; *P(=O)(O)O]
groups;
A is a bond, —OC(═O)—*, 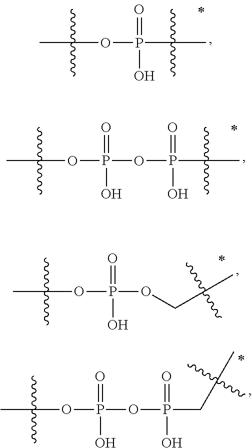 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
and
D is a Drug moiety comprising an N or an O, wherein D is connected to A via a direct bond from A to the N or the O of the Drug moiety.

6. The compound of claim 1, or pharmaceutically acceptable salt thereof, wherein:
R¹ is 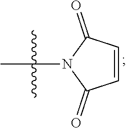 [SMILES: *N1C(=O)C=CC1=O]
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 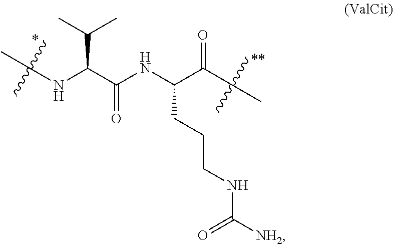 [SMILES: *N[C@H](C(=O)N[C@@H](CCCNC(N)=O)C(*)=O)C(C)C]
where the * of Lp indicates the attachment point to L₁ and the ** of Lp indicates the attachment point to the —NH— group of G;
L₃ is a spacer moiety having the structure 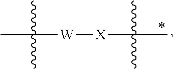
where
W is —CH₂O—**, —CH₂N(R^{b})C(═O)O—**, —NHC(═O)CH₂NHC(═O)O—** —CH₂N(X—R²)C(═O)O—**, —C(═O)N(X—R²)—**, —CH₂N(X—R²)C(═O)—** —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
X is a bond, triazolyl or ***—CH₂-triazolyl*, wherein the *** of X indicates the point of attachment to W and the * of X indicates the point of attachment to R²;
and
the * of L₃ indicates the point of attachment to R²;
R² is a hydrophilic moiety selected from polyethylene glycol, polyalkylene glycol, a sugar, an oligosaccharide, a polysarcosine, a polypeptide or C₂-C₆alkyl substituted with 1 to 3 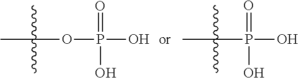 [SMILES: *OP(=O)(O)O ; *P(=O)(O)O]
groups;
A is a bond, —OC(═O)—*, 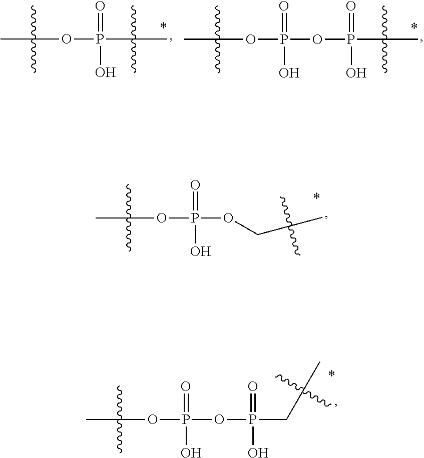 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
and
D is a Drug moiety comprising an N or an O, wherein D is connected to A via a direct bond from A to the N or the O of the Drug moiety.

7. The compound of claim 1, or pharmaceutically acceptable salt thereof, wherein:
R¹ is 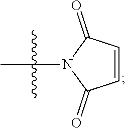 [SMILES: *N1C(=O)C=CC1=O]
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 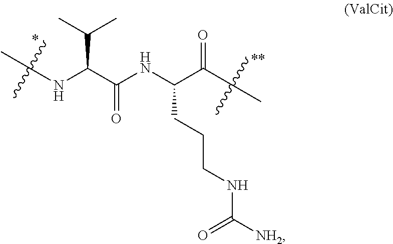 [SMILES: *N[C@H](C(=O)N[C@@H](CCCNC(N)=O)C(*)=O)C(C)C]
where the * of Lp indicates the attachment point to L₁ and the ** of Lp indicates the attachment point to the —NH— group of G;
L₃ is a spacer moiety having the structure 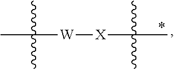
where
W is —CH₂O—**, —CH₂N(R^{b})C(═O)O—**, —NHC(═O)CH₂NHC(═O)O—** —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
X is a bond, triazolyl or ***—CH₂-triazolyl-*, wherein the *** of X indicates the point of attachment to W and the * of X indicates the point of attachment to R²;
and
the * of L₃ indicates the point of attachment to R²;
R² is a hydrophilic moiety selected from polyethylene glycol, polyalkylene glycol, a sugar, an oligosaccharide, a polysarcosine, a polypeptide or C₂-C₆alkyl substituted with 1 to 3 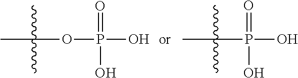 [SMILES: *OP(=O)(O)O ; *P(=O)(O)O]
groups;
A is a bond or —OC(═O)*, in which * indicates the attachment point to D; and
D is a Drug moiety comprising an N or an O, wherein D is connected to A via a direct bond from A to the N or the O of the Drug moiety.

8. The compound of claim 1, or pharmaceutically acceptable salt thereof, wherein:
R¹ is 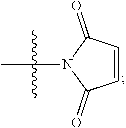 [SMILES: *N1C(=O)C=CC1=O]
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 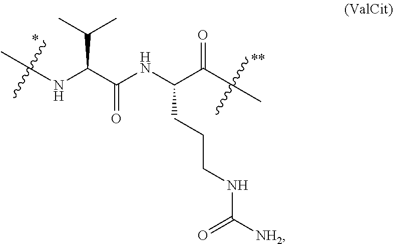 [SMILES: *N[C@H](C(=O)N[C@@H](CCCNC(N)=O)C(*)=O)C(C)C]
where the * of Lp indicates the attachment point to L₁ and the ** of Lp indicates the attachment point to the —NH— group of G;
L₃ is a spacer moiety having the structure 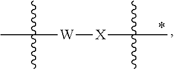
where
W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
X is ***—CH₂-triazolyl-*, wherein the *** of X indicates the point of attachment to W and the * of X indicates the point of attachment to R²;
and
the * of L₃ indicates the point of attachment to R²;
R² is a hydrophilic moiety selected from polyethylene glycol, polyalkylene glycol, a sugar, an oligosaccharide, a polysarcosine, a polypeptide or C₂-C₆alkyl substituted with 1 to 3 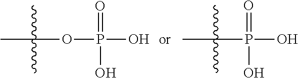 [SMILES: *OP(=O)(O)O ; *P(=O)(O)O]
groups;
A is a bond or —OC(═O)*, in which * indicates the attachment point to D; and
D is a Drug moiety comprising an N or an O, wherein D is connected to A via a direct bond from A to the N or the O of the Drug moiety.

9. The compound of claim 1, or pharmaceutically acceptable salt thereof, wherein:
R¹ is 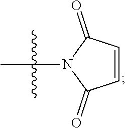 [SMILES: *N1C(=O)C=CC1=O]
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 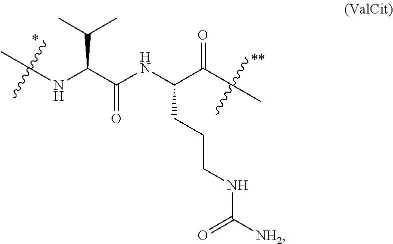 [SMILES: *N[C@H](C(=O)N[C@@H](CCCNC(N)=O)C(*)=O)C(C)C]
where the * of Lp indicates the attachment point to L₁ and the ** of Lp indicates the attachment point to the —NH— group of G;
L₃ is a spacer moiety having the structure 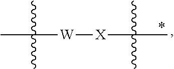
where
W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
X is ***—CH₂-triazolyl-*, wherein the *** of X indicates the point of attachment to W and the * of X indicates the point of attachment to R²;
and
the * of L₃ indicates the point of attachment to R²;
R² is a polyethylene glycol;
A is a bond or —OC(═O)*, in which * indicates the attachment point to D; and
D is a Drug moiety comprising an N or an O, wherein D is connected to A via a direct bond from A to the N or the O of the Drug moiety.

10. The compound of claim 1, or pharmaceutically acceptable salt thereof, having the structure: 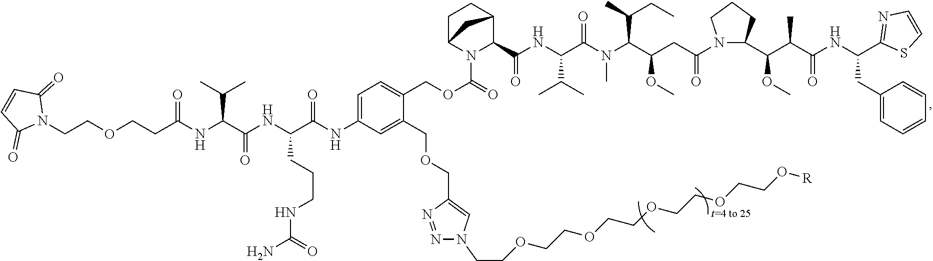
where
R is H, —CH₃ or —CH₂CH₂C(═O)OH.

11. The compound of claim 1, or pharmaceutically acceptable salt thereof, having the structure: 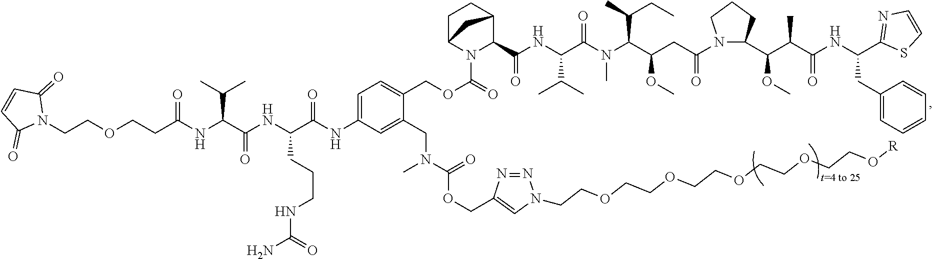
where
R is H, —CH₃ or —CH₂CH₂C(═O)OH.

12. The compound of claim 1, or pharmaceutically acceptable salt thereof, having the structure: 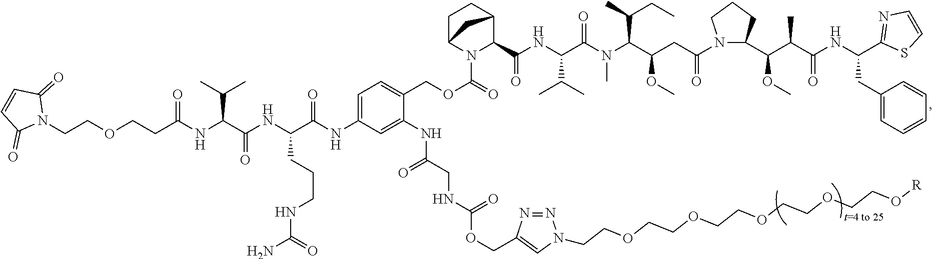
where
R is H, —CH₃ or —CH₂CH₂C(═O)OH.

13. The compound of claim 1, or pharmaceutically acceptable salt thereof, having the structure: 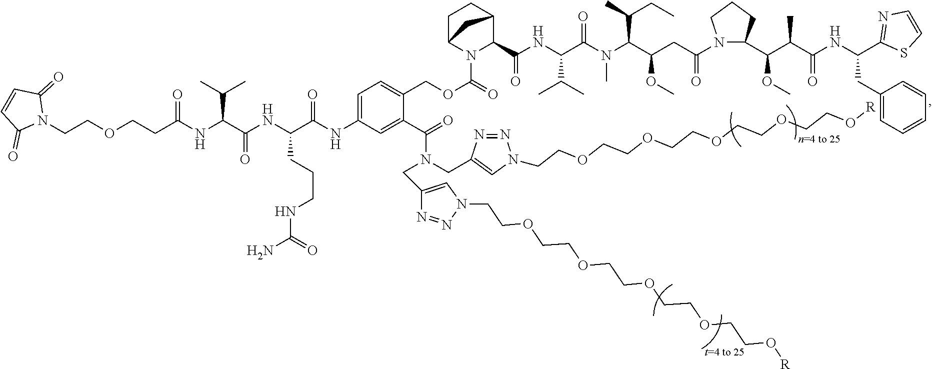
where each R is independently selected from H, —CH₃ or —CH₂CH₂C(═O)OH.

14. The compound of claim 1, or pharmaceutically acceptable salt thereof, having the structure: 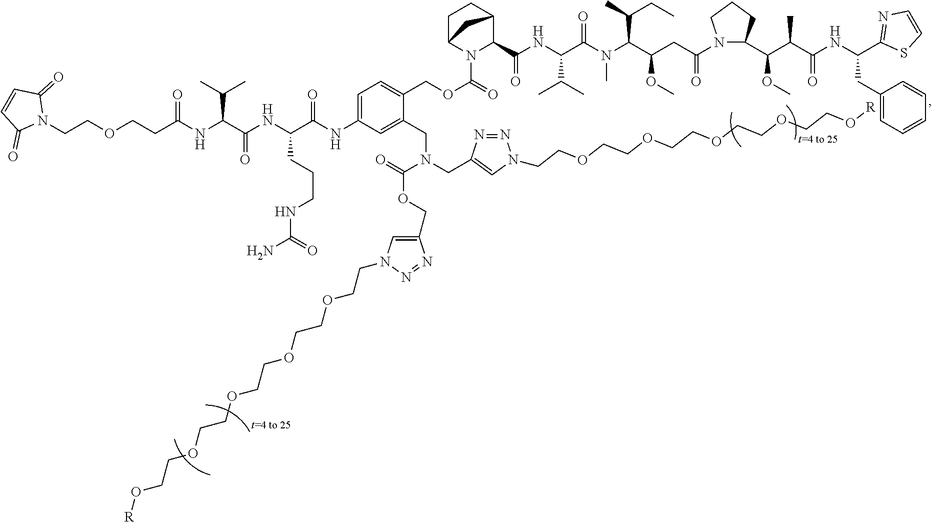
where each R is independently selected from H, —CH₃ or —CH₂CH₂C(═O)OH.

15. The compound of claim 1, or pharmaceutically acceptable salt thereof, having the structure: 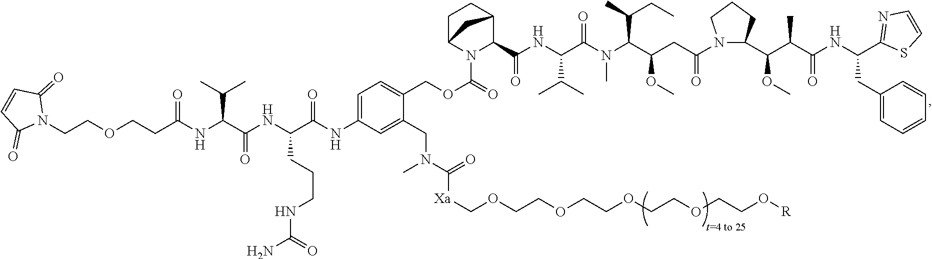
where Xa is —CH₂—, —OCH₂—, —NHCH₂— or —NRCH₂— and each R independently is H, —CH₃ or —CH₂CH₂C(═O)OH.

16. The compound of claim 1, or pharmaceutically acceptable salt thereof, having the structure: 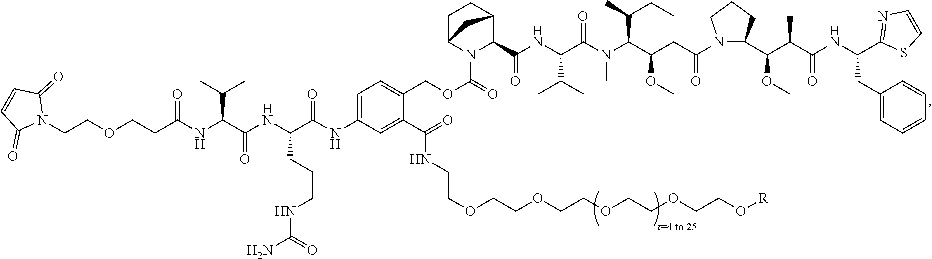
where
R is H, —CH₃ or —CH₂CH₂C(═O)OH.

17. The compound of claim 1, or pharmaceutically acceptable salt thereof, having the structure: 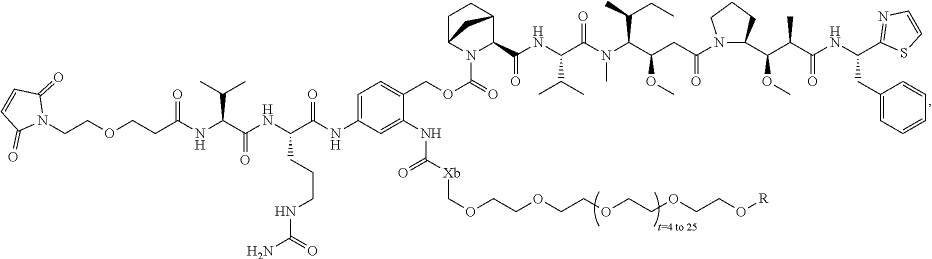
where Xb is —CH₂—, —OCH₂—, —NHCH₂— or —NRCH₂— and each R independently is H, —CH₃ or —CH₂CH₂C(═O)OH.

18. The compound of claim 1, or pharmaceutically acceptable salt thereof, having the structure:

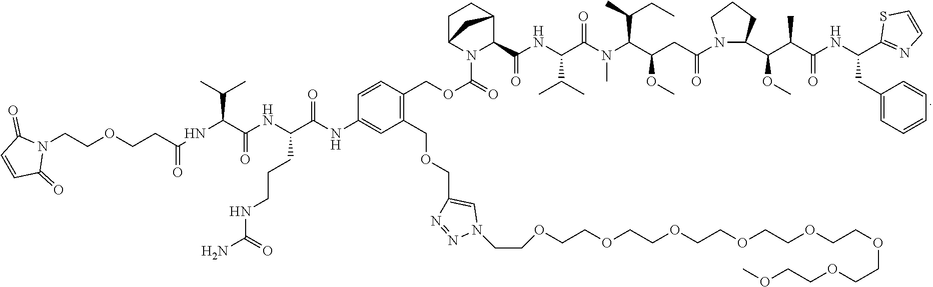 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOC)nn1)C(C)C]

19. The compound of claim 1, or pharmaceutically acceptable salt thereof, having the structure:

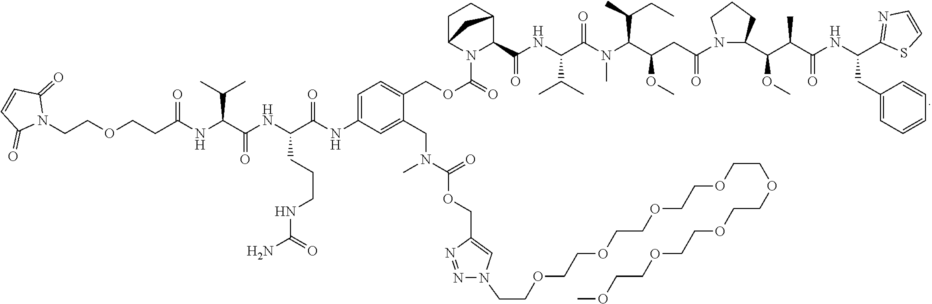 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOC)nn1)C(C)C]

20. The compound of claim 1, or pharmaceutically acceptable salt thereof, having the structure:

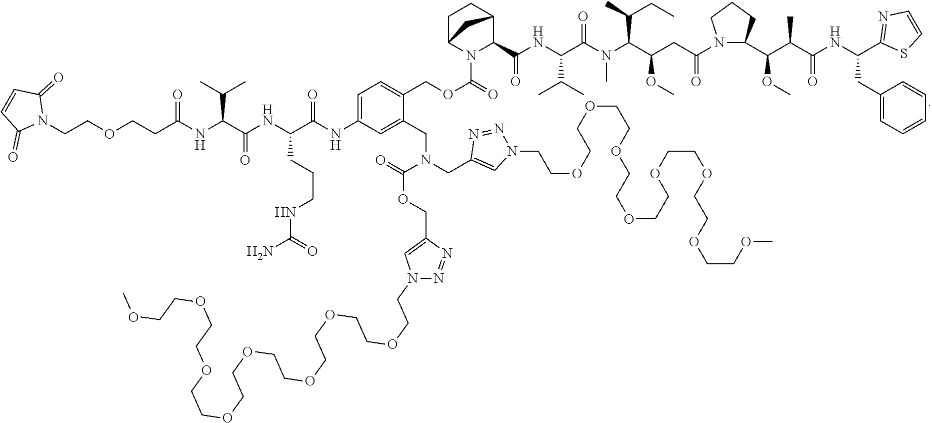 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1)C(C)C]

21. The compound of claim 1, or pharmaceutically acceptable salt thereof, having the structure:

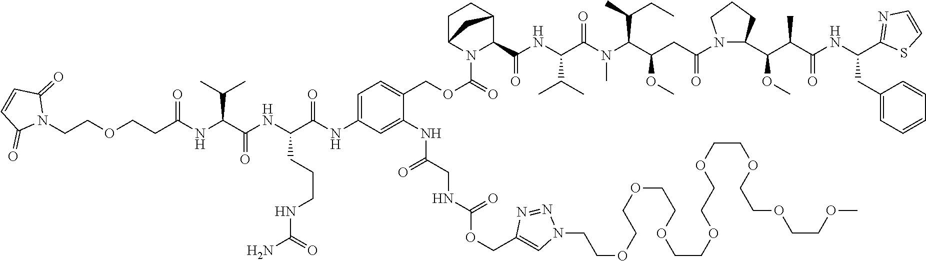 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1NC(=O)CNC(=O)OCc1cn(CCOCCOCCOCCOCCOCCOCCOCCOC)nn1)C(C)C]

22. The compound of claim 1, or pharmaceutically acceptable salt thereof, having the structure:

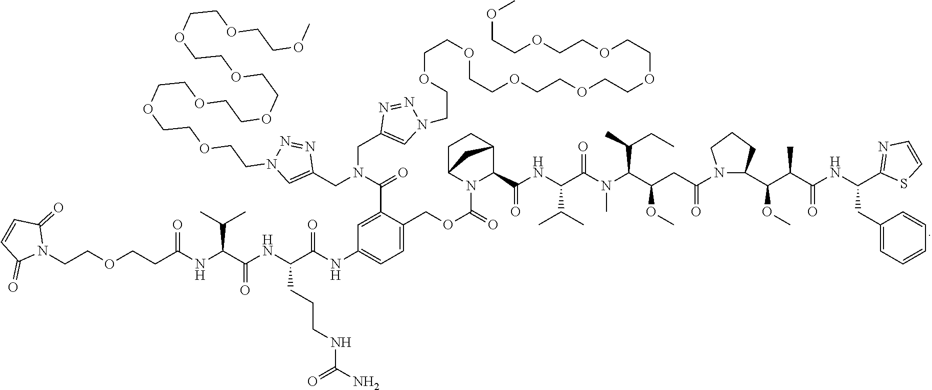 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCOCCN2C(=O)C=CC2=O)C(C)C)cc1C(=O)N(Cc1cn(CCOCCOCCOCCOCCOCCOCCOCCOC)nn1)Cc1cn(CCOCCOCCOCCOCCOCCOCCOCCOC)nn1)C(C)C]

23. A linker having the structure of Formula (V), 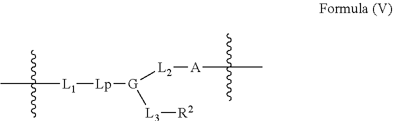
wherein
L₁ is a bridging spacer;
Lp is a bivalent peptide spacer;
G-L₂-A is a self-immolative spacer;
R² is a hydrophilic moiety;
L₂ is a bond, a methylene, a neopentylene or a C₂-C₃alkenylene;
A is a bond, **—OC(O)—, 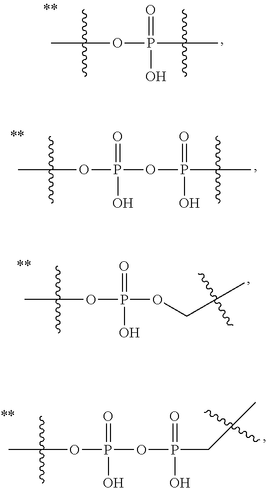 [SMILES: *OP(*)(=O)O ; *OP(=O)(O)OP(*)(=O)O ; *COP(=O)(O)O* ; *CP(=O)(O)OP(=O)(O)O*]
**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂,
and
L₃ is a spacer moiety.

24. The linker of claim 23, wherein:
L₁ is a bridging spacer;
Lp is a bivalent peptide spacer comprising one to four amino acid residues;
G-L₂-A is a self-immolative spacer;
R² is a hydrophilic moiety;
L₂ is a bond, a methylene, a neopentylene or a C₂-C₃alkenylene;
A is a bond, **—OC(═O)—, 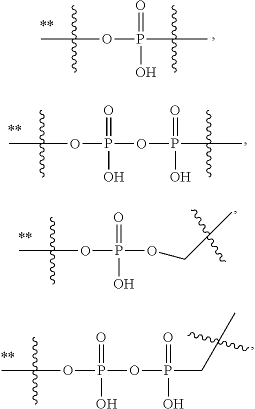
**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂,
and
L₃ is a spacer moiety.

25. The linker of claim 23, wherein:
L₁ is a bridging spacer;
Lp is a bivalent peptide spacer comprising one to four amino acid residues;
the 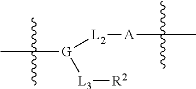
group is selected from: 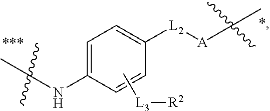
wherein the * of 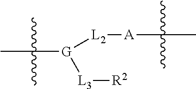
indicates the point of attachment to an N or a O of the Drug moiety, the *** of 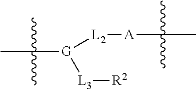
indicates the point of attachment to Lp;
R² is a hydrophilic moiety;
L₂ is a bond, a methylene, a neopentylene or a C₂-C₃alkenylene;
A is a bond, **—OC(═O)—, 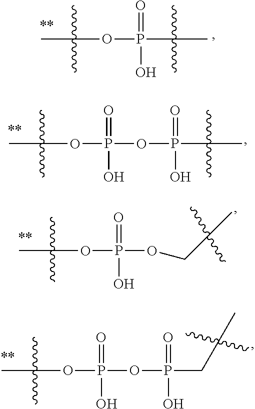
**—OC(═O)N(CH₃)CH₂CH₂N(CH₃)C(═O)— or **—OC(═O)N(CH₃)C(R^{a})₂C(R^{a})₂N(CH₃)C(═O)—, wherein each R^{a} is independently selected from H, C₁-C₆alkyl or a C₃-C₈cycloalkyl and wherein the ** of A indicates the point of attachment to L₂,
and
L₃ is a spacer moiety.

26. The linker of claim 23 having the structure of Formula (VI), 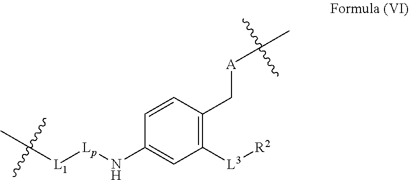
wherein
L₁ is a bridging spacer;
Lp is a bivalent peptide spacer;
R² is a hydrophilic moiety;
A is a bond, —OC(═O)—, 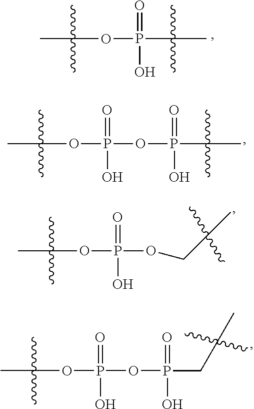
—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl,
and
L₃ is a spacer moiety.

27. The linker of claim 23, wherein:
L₁ is a bridging spacer;
Lp is a bivalent peptide spacer comprising one to four amino acid residues;
R² is a hydrophilic moiety;
A is a bond, —OC(═O)—, 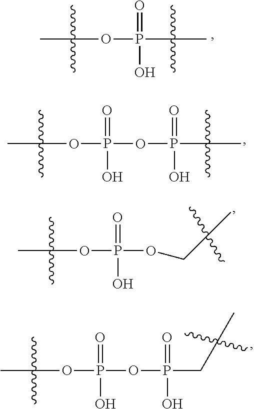
—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl,
and
L₃ is a spacer moiety.

28. The linker of claim 23, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp;
R² is a hydrophilic moiety selected from polyethylene glycol, polyalkylene glycol, a sugar, an oligosaccharide, a polysarcosine, a polypeptide or C₂-C₆alkyl substituted with 1 to 3 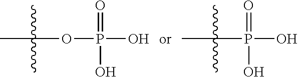 [SMILES: *OP(=O)(O)O ; *P(=O)(O)O]
groups;
each R³ is independently selected from H and C₁-C₆alkyl;
X₁ is 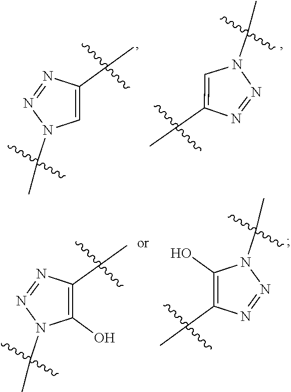 [SMILES: *c1cn(*)nn1 ; *c1cn(*)nn1 ; *c1nnn(*)c1O ; *c1nnn(*)c1O]
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising one to four amino acid residues independently selected from glycine, valine, citrulline, lysine, isoleucine, phenylalanine, methionine, asparagine, proline, alanine, leucine, tryptophan, and tyrosine;
A is a bond, —OC(═O)—, 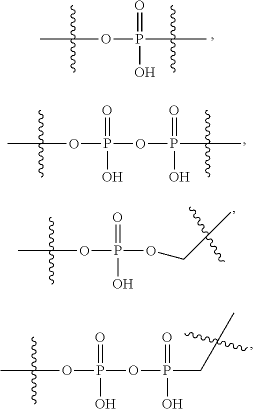
—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl;
L₃ is a spacer moiety having the structure 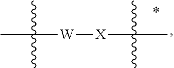
where
(i) W is —CH₂O—**, —CH₂N(R^{b})C(═O)O—**, —NHC(═O)C(R^{b})₂NHC(═O)O—**, —NHC(═O)C(R^{b})₂NH—**, —NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—**, —NH—, or —CH₂N(R^{b})C(═O)CH₂—**, wherein each R^{b} is independently selected from H, C₁-C₆alkyl or C₃-C₈cycloalkyl and wherein the ** of W indicates the point of attachment to X;
X is a bond or ***—CH₂-triazolyl-*, wherein the *** of X indicates the point of attachment to W and the * of X indicates the point of attachment to R²; or
(ii) W is —CH₂O—**, —CH₂N(R^{b})C(═O)O—**, —NHC(═O)C(R^{b})₂NHC(═O)O—**, —NHC(═O)C(R^{b})₂NH—**, NHC(═O)C(R^{b})₂NHC(═O)—**, —CH₂N(X—R²)C(═O)O—**, —C(═O)N(X—R²)—**, —CH₂N(X—R²)C(═O)—** —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
X is ***—CH₂-triazolyl-C₁₋₄ alkylene-OC(O)NHS(O)₂NH—*, ***—C₄₋₆ cycloalkylene-OC(O)NHS(O)₂NH—*, ***—(CH₂CH₂O)ₙ—C(O)NHS(O)₂NH—*, ***—(CH₂CH₂O)ₙ—C(O)NHS(O)₂NH—(CH₂CH₂O)ₙ—*, or ***—CH₂-triazolyl-C₁₋₄ alkylene-OC(O)NHS(O)₂NH—(CH₂CH₂O)ₙ—*, wherein each n independently is 1, 2, or 3, the *** of X indicates the point of attachment to W and the * of X indicates the point of attachment to R²;
and
the * of L₃ indicates the point of attachment to R².

29. The linker of claim 23, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 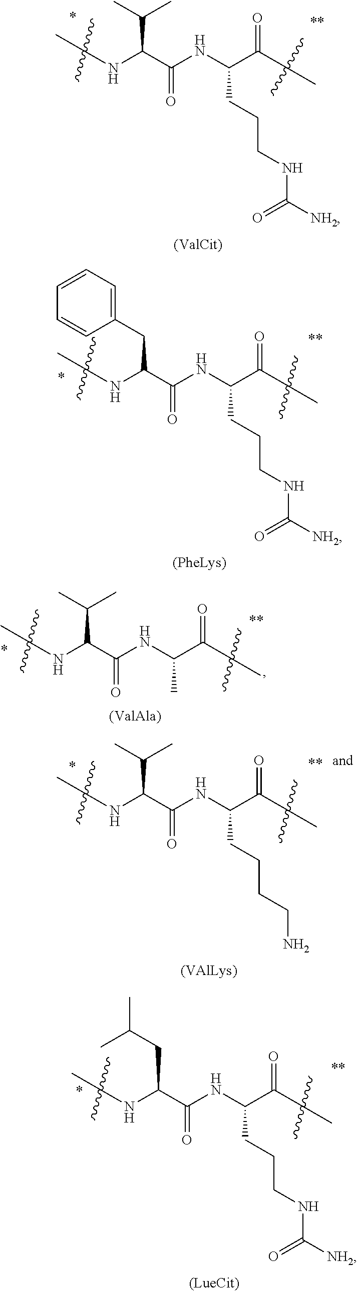
where the * of Lp indicates the attachment point to L₁ and the ** of Lp indicates the attachment point to the —NH— group;
L₃ is a spacer moiety having the structure 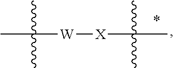
where
W is —CH₂O—**, —CH₂N(R^{b})C(═O)O—**, —NHC(═O)CH₂NHC(═O)O—** —CH₂N(X—R²)C(═O)O—**, —C(═O)N(X—R²)—**, —CH₂N(X—R²)C(═O)—** —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
X is a bond, triazolyl or ***—CH₂-triazolyl-*, wherein the *** of X indicates the point of attachment to W and the * of X indicates the point of attachment to R²;
and
the * of L₃ indicates the point of attachment to R²;
R² is a hydrophilic moiety selected from polyethylene glycol, polyalkylene glycol, a sugar, an oligosaccharide, a polysarcosine, a polypeptide or C₂-C₆alkyl substituted with 1 to 3 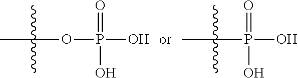 [SMILES: *OP(=O)(O)O ; *P(=O)(O)O]
groups;
A is a bond, —OC(═O)—, 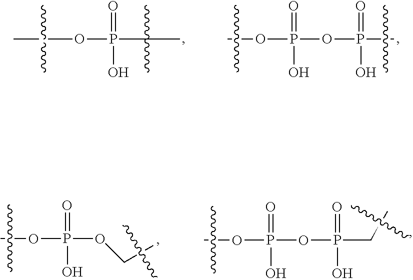
—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl.

30. The linker of claim 23, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 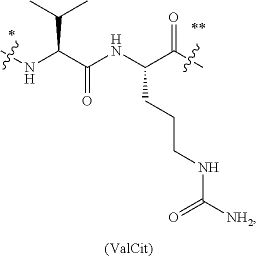
where the * of Lp indicates the attachment point to L₁ and the ** of Lp indicates the attachment point to the —NH— group;
L₃ is a spacer moiety having the structure 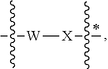
where
W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
X is a bond, triazolyl or ***—CH₂-triazolyl*, wherein the *** of X indicates the point of attachment to W and the * of X indicates the point of attachment to R²;
and
the * of L₃ indicates the point of attachment to R²;
R² is a hydrophilic moiety selected from polyethylene glycol, polyalkylene glycol, a sugar, an oligosaccharide, a polysarcosine, a polypeptide or C₂-C₆alkyl substituted with 1 to 3 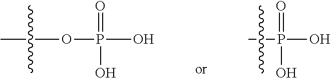
groups;
A is a bond, —OC(═O)—, 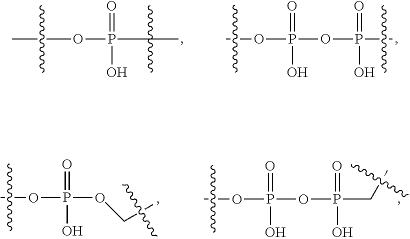
—OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl.

31. The linker of claim 23, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 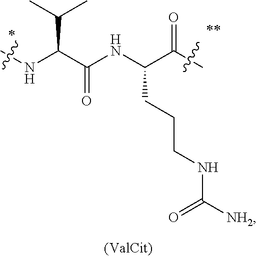
where the * of Lp indicates the attachment point to L₁ and the ** of Lp indicates the attachment point to the —NH— group;
L₃ is a spacer moiety having the structure 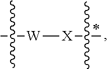
where
W is —CH₂O—**, —CH₂N(R^{b})C(═O)O—**, —NHC(═O)CH₂NHC(═O)O—** —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
X is a bond or ***—CH₂-triazolyl-*, wherein the *** of X indicates the point of attachment to W and the * of X indicates the point of attachment to R²;
and
the * of L₃ indicates the point of attachment to R²;
R² is a hydrophilic moiety selected from polyethylene glycol, polyalkylene glycol, a sugar, an oligosaccharide, a polysarcosine, a polypeptide or C₂-C₆alkyl substituted with 1 to 3 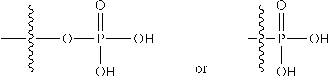
groups;
and
A is a bond or —OC(═O)—.

32. The linker of claim 23, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 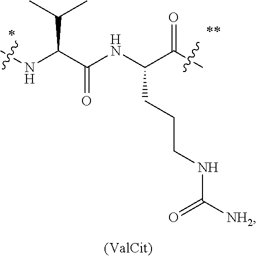
where the * of Lp indicates the attachment point to L₁ and the ** of Lp indicates the attachment point to the —NH— group;
L₃ is a spacer moiety having the structure 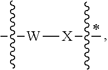
where
W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
X is ***—CH₂-triazolyl-*, wherein the *** of X indicates the point of attachment to W and the * of X indicates the point of attachment to R²;
and
the * of L₃ indicates the point of attachment to R²;
R² is a hydrophilic moiety selected from polyethylene glycol, polyalkylene glycol, a sugar, an oligosaccharide, a polysarcosine, a polypeptide or C₂-C₆alkyl substituted with 1 to 3 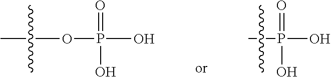
groups;
and
A is a bond or —OC(═O)—.

33. The linker of claim 23, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from 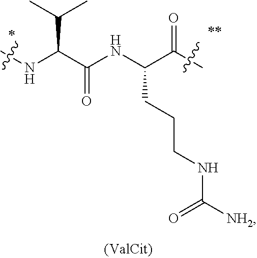
where the * of Lp indicates the attachment point to L₁ and the ** of Lp indicates the attachment point to the —NH— group;
L₃ is a spacer moiety having the structure 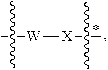
where
W is —CH₂O—**, —CH₂N(R^{b})C(═O)O—**, —NHC(═O)CH₂NHC(═O)O—** —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
X is ***—CH₂-triazolyl-*, wherein the *** of X indicates the point of attachment to W and the * of X indicates the point of attachment to R²;
and
the * of L₃ indicates the point of attachment to R²;
R² is a polyethylene glycol;
and
A is a bond or —OC(═O)—.

34. The linker of claim 23, having the structure: 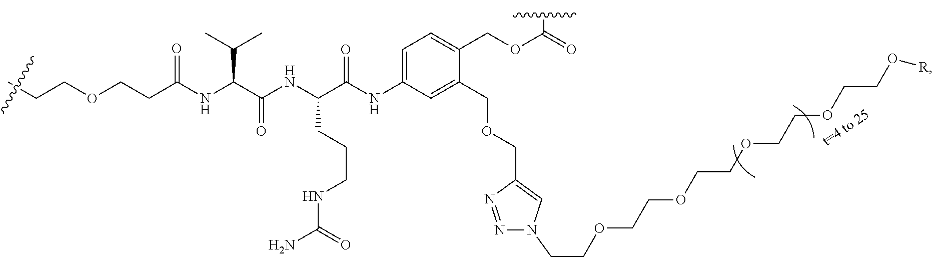
where
R is H, —CH₃ or —CH₂CH₂C(═O)OH.

35. The linker of claim 23, having the structure: 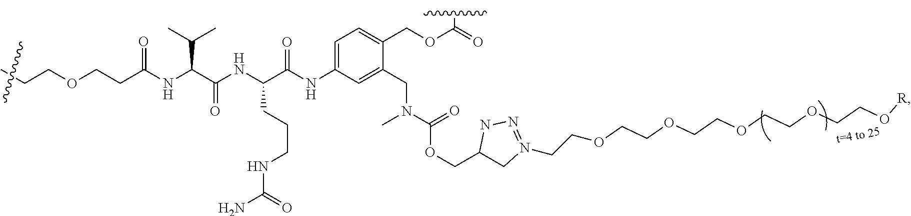
where
R is H, —CH₃ or —CH₂CH₂C(═O)OH.

36. The linker of claim 23, having the structure: 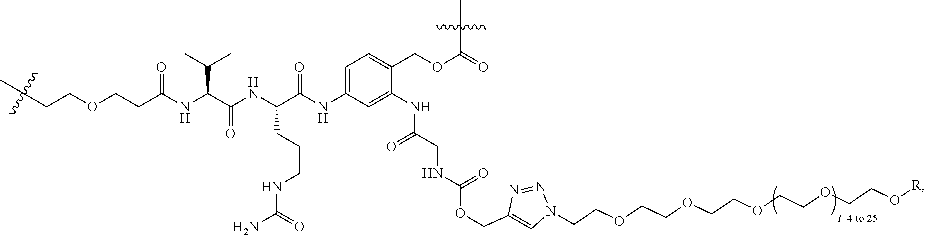
where
R is H, —CH₃ or —CH₂CH₂C(═O)OH.

37. The linker of claim 23, having the structure: 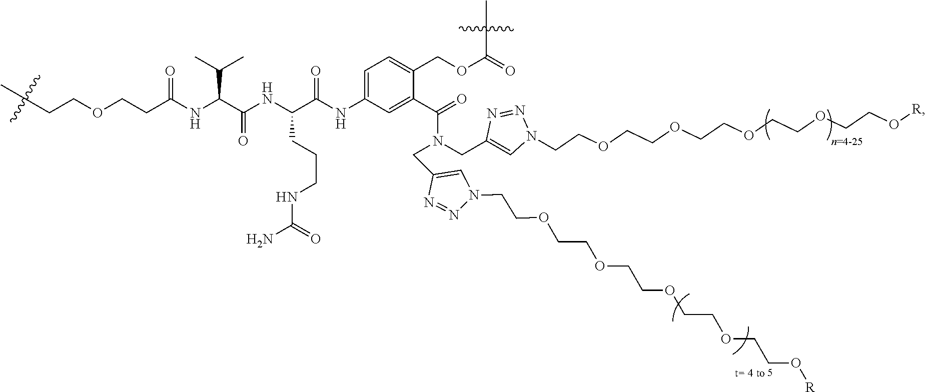
where
each R is independently selected from H, —CH₃ or —CH₂CH₂C(═O)OH.

38. The linker of claim 23, having the structure: 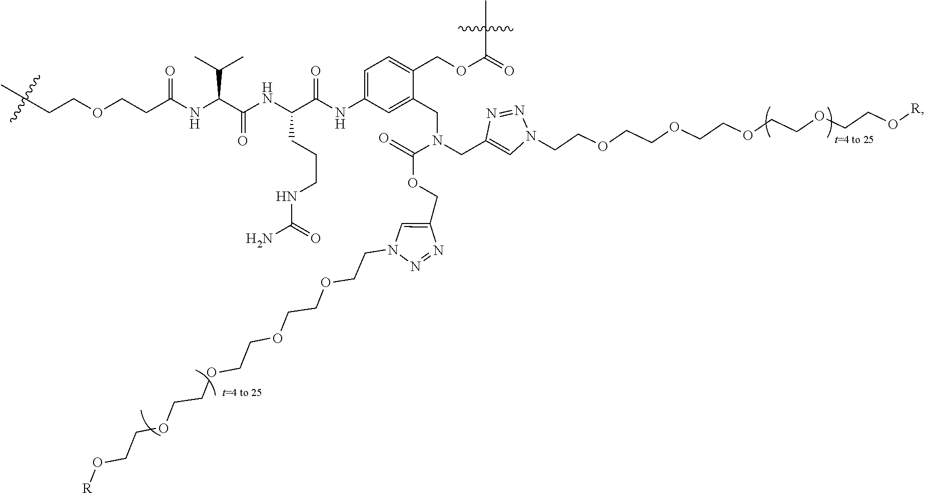
where
each R is independently selected from H, —CH₃ or —CH₂CH₂C(═O)OH.

39. The linker of claim 23, having the structure: 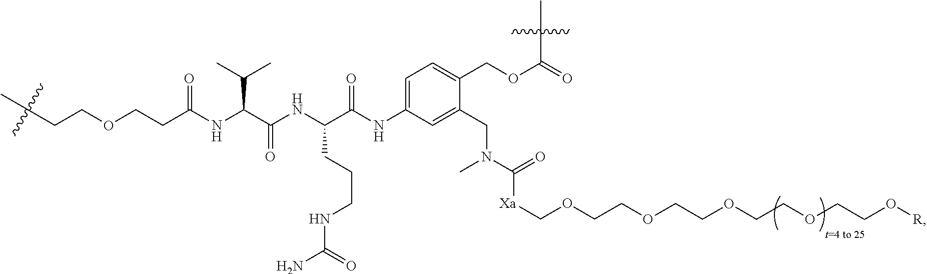
where
Xa is —CH₂—, —OCH₂—, —NHCH₂— or —NRCH₂— and each R independently is H, —CH₃ or —CH₂CH₂C(═O)OH.

40. The linker of claim 23, having the structure: 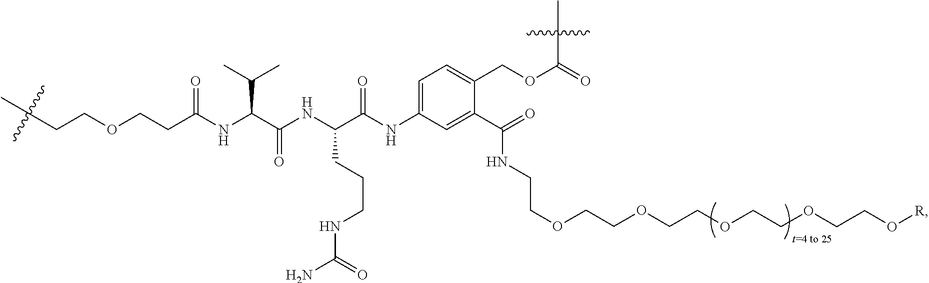
where
R is H, —CH₃ or —CH₂CH₂C(═O)OH.

41. The linker of claim 23, having the structure: 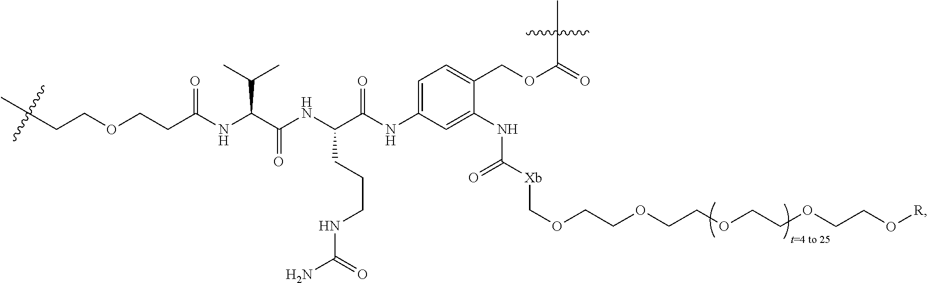
where
Xb is —CH₂—, —OCH₂—, —NHCH₂— or —NRCH₂— and each R independently is H, —CH₃ or —CH₂CH₂C(═O)OH.

42. The linker of claim 23, having the structure:

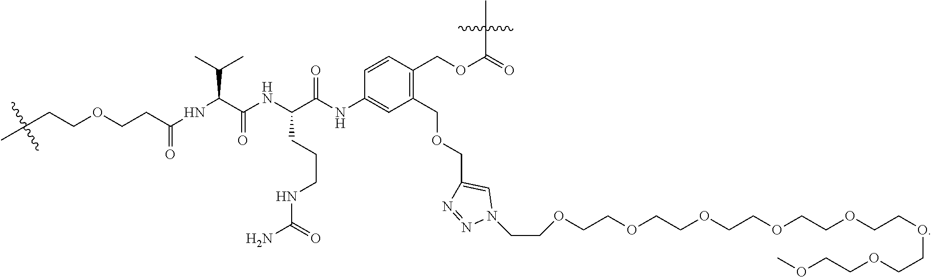 [SMILES: *CCOCCC(=O)N[C@H](C(=O)N[C@@H](CCCNC(N)=O)C(=O)Nc1ccc(COC(*)=O)c(COCc2cn(CCOCCOCCOCCOCCOCCOCCOCCOC)nn2)c1)C(C)C]

43. The linker of claim 23, having the structure:

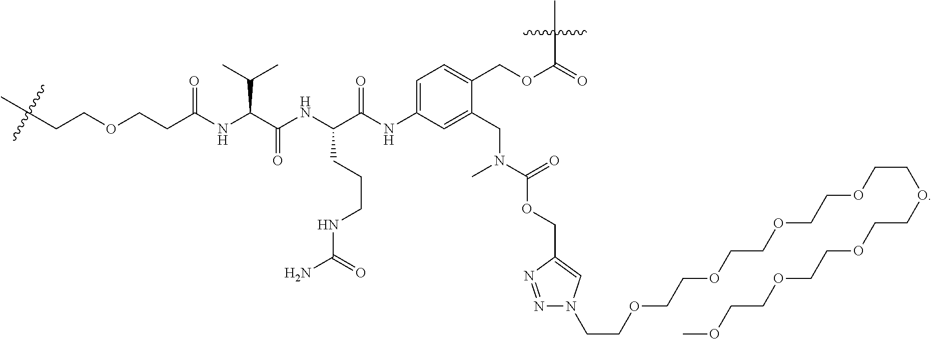 [SMILES: *CCOCCC(=O)N[C@H](C(=O)N[C@@H](CCCNC(N)=O)C(=O)Nc1ccc(COC(*)=O)c(CN(C)C(=O)OCc2cn(CCOCCOCCOCCOCCOCCOCCOCCOC)nn2)c1)C(C)C]

44. The linker of claim 23, having the structure:

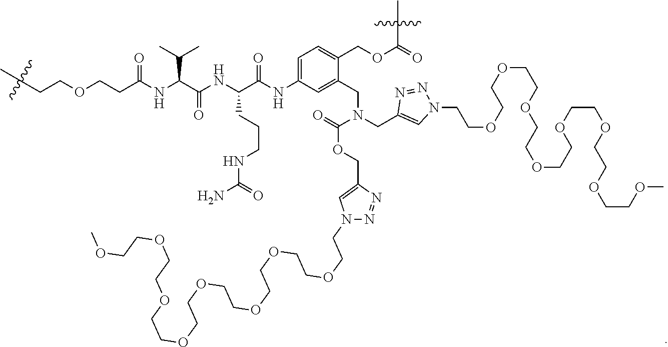 [SMILES: *CCOCCC(=O)N[C@H](C(=O)N[C@@H](CCCNC(N)=O)C(=O)Nc1ccc(COC(*)=O)c(CN(Cc2cn(CCOCCOCCOCCOCCOCCOCCOCCOC)nn2)C(=O)OCc2cn(CCOCCOCCOCCOCCOCCOCCOCCOC)nn2)c1)C(C)C]

45. The linker of claim 23, having the structure:

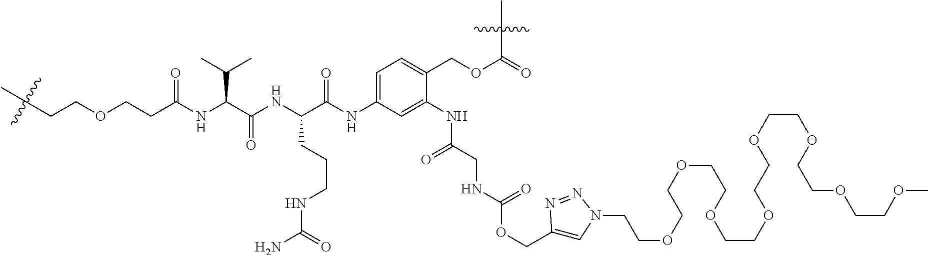 [SMILES: *CCOCCC(=O)N[C@H](C(=O)N[C@@H](CCCNC(N)=O)C(=O)Nc1ccc(COC(*)=O)c(NC(=O)CNC(=O)OCc2cn(CCOCCOCCOCCOCCOCCOCCOCCOC)nn2)c1)C(C)C]

46. The linker of claim 23, having the structure:

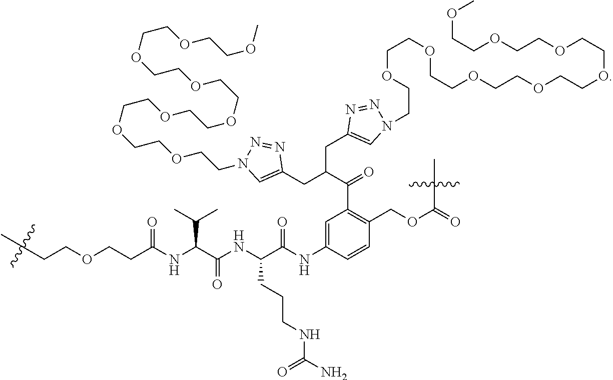 [SMILES: *CCOCCC(=O)N[C@H](C(=O)N[C@@H](CCCNC(N)=O)C(=O)Nc1ccc(COC(*)=O)c(C(=O)C(Cc2cn(CCOCCOCCOCCOCCOCCOCCOCCOC)nn2)Cc2cn(CCOCCOCCOCCOCCOCCOCCOCCOC)nn2)c1)C(C)C]

47. A conjugate of Formula (III): 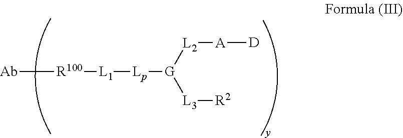
wherein:
Ab is an antibody or fragment thereof;
R¹⁰⁰ is a coupling group;
L₁ is a bridging spacer;
Lp is a bivalent peptide linker;
G-L₂-A is a self-immolative spacer;
R² is a hydrophilic moiety;
L₂ is a bond, a methylene, a neopentylene or a C₂-C₃alkenylene;
A is a bond, —OC(═O)—*, 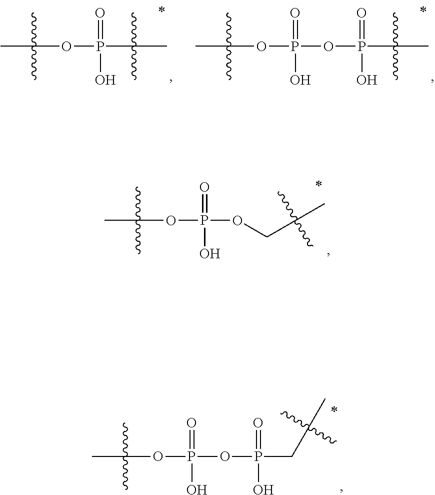 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.

48. A conjugate of Formula (IV): 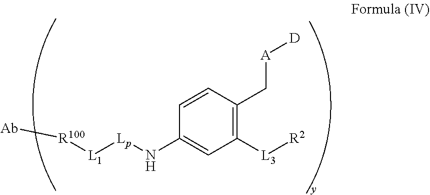
wherein:
Ab is an antibody or fragment thereof;
R¹⁰⁰ is a coupling group;
L₁ is a bridging spacer;
Lp is a bivalent peptide linker comprising one to four amino acid residues;
R² is a hydrophilic moiety;
A is a bond, —OC(═O)—*, 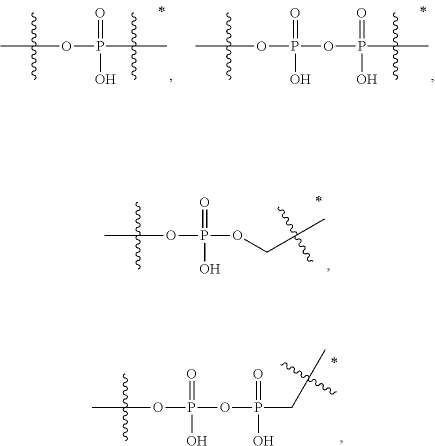 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl or a C₃-C₈cycloalkyl and the * of A indicates the point of attachment to D;
L₃ is a spacer moiety;
D is a Drug moiety comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.


SEQUENCE LISTING (166 sequences)

SEQ ID NO: 1 - protein, 7 aa, Artificial Sequence
  source: /note=“Description of Artificial Sequence: Synthetic peptide”
SQSAAWN

SEQ ID NO: 2 - protein, 18 aa, Artificial Sequence
  source: /note=“Description of Artificial Sequence: Synthetic peptide”
RIYYRSKWYNDYALSVKS

SEQ ID NO: 3 - protein, 11 aa, Artificial Sequence
  source: /note=“Description of Artificial Sequence: Synthetic peptide”
GEGYGREGFAI

SEQ ID NO: 4 - protein, 9 aa, Artificial Sequence
  source: /note=“Description of Artificial Sequence: Synthetic peptide”
GDSVSSQSA

SEQ ID NO: 5 - protein, 7 aa, Artificial Sequence
  source: /note=“Description of Artificial Sequence: Synthetic peptide”
YYRSKWY

SEQ ID NO: 6 - protein, 11 aa, Artificial Sequence
  source: /note=“Description of Artificial Sequence: Synthetic peptide”
GEGYGREGFAI

SEQ ID NO: 7 - protein, 123 aa, Artificial Sequence
  source: /note=“Description of Artificial Sequence: Synthetic polypeptide”
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSS

SEQ ID NO: 8 - DNA, 369 bp, Artificial Sequence
  source: /note=“Description of Artificial Sequence: Synthetic polynucleotide”
caggtgcagctgcagcagtcaggccctggcctggtcaagcctagtcagaccctgagcctgacctgcgctattagcggcgatagtgtgtctagtcagtcagccgcctggaactggattagacagtcaccctctaggggcctggagtggctgggtagaatctactataggtctaagtggtataacgactacgccctgagcgtgaagtctaggatcactattaaccccgacacctctaagaatcagtttagcctgcagctgaatagcgtgacccccgaggacaccgccgtctactactgcgctagaggcgagggctacggtagagagggcttcgctatctggggtcagggcaccctggtcaccgtgtctagc

SEQ ID NO: 9 - protein, 453 aa, Artificial Sequence
  source: /note=“Description of Artificial Sequence: Synthetic polypeptide”
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1359 bp, Artificial Sequence
  source: /note=“Description of Artificial Sequence: Synthetic polynucleotide”
caggtgcagctgcagcagtcaggccctggcctggtcaagcctagtcagaccctgagcctgacctgcgctattagcggcgatagtgtgtctagtcagtcagccgcctggaactggattagacagtcaccctctaggggcctggagtggctgggtagaatctactataggtctaagtggtataacgactacgccctgagcgtgaagtctaggatcactattaaccccgacacctctaagaatcagtttagcctgcagctgaatagcgtgacccccgaggacaccgccgtctactactgcgctagaggcgagggctacggtagagagggcttcgctatctggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 11 - protein, 11 aa, Artificial Sequence
  source: /note=“Description of Artificial Sequence: Synthetic peptide”
RASQTISNTLA

SEQ ID NO: 12 - protein, 7 aa, Artificial Sequence
  source: /note=“Description of Artificial Sequence: Synthetic peptide”
AASNLQS

SEQ ID NO: 13 - protein, 8 aa, Artificial Sequence
  source: /note=“Description of Artificial Sequence: Synthetic peptide”
QQYLSWFT

SEQ ID NO: 14 - protein, 7 aa, Artificial Sequence
  source: /note=“Description of Artificial Sequence: Synthetic peptide”
SQTISNT

SEQ ID NO: 15 - protein, 3 aa, Artificial Sequence
  source: /note=“Description of Artificial Sequence: Synthetic peptide”
AAS

SEQ ID NO: 16 - protein, 5 aa, Artificial Sequence
  source: /note=“Description of Artificial Sequence: Synthetic peptide”
YLSWF

SEQ ID NO: 17 - protein, 106 aa, Artificial Sequence
  source: /note=“Description of Artificial Sequence: Synthetic polypeptide”
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIK

SEQ ID NO: 18 - DNA, 318 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagagcctctcagactatctctaacaccctggcctggtatcagcagaagcccggtaaagcccctaagctgctgatctacgccgcctctaacctgcagtcaggcgtgccctctaggtttagcggtagcggtagtggcaccgacttcaccctgactattagtagcctgcagcccgaggacttcgctacctactactgtcagcagtacctgagctggttcaccttcggtcagggcactaaggtcgagattaag

SEQ ID NO: 19 - protein, 213 aa, Artificial Sequence
  source: /note=“Description of Artificial Sequence: Synthetic polypeptide”
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 639 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagagcctctcagactatctctaacaccctggcctggtatcagcagaagcccggtaaagcccctaagctgctgatctacgccgcctctaacctgcagtcaggcgtgccctctaggtttagcggtagcggtagtggcaccgacttcaccctgactattagtagcctgcagcccgaggacttcgctacctactactgtcagcagtacctgagctggttcaccttcggtcagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 21 - protein, 5 aa, Artificial Sequence
  source: /note=“Description of Artificial Sequence: Synthetic peptide”
DHTIH

SEQ ID NO: 22 - protein, 17 aa, Artificial Sequence
  source: /note=“Description of Artificial Sequence: Synthetic peptide”
YIYPRSGSINYNEKFKG

SEQ ID NO: 23 - protein, 9 aa, Artificial Sequence
  source: /note=“Description of Artificial Sequence: Synthetic peptide”
RNLFLPMEY

SEQ ID NO: 24 - protein, 7 aa, Artificial Sequence
  source: /note=“Description of Artificial Sequence: Synthetic peptide”
GYTFTDH

SEQ ID NO: 25 - protein, 6 aa, Artificial Sequence
  source: /note=“Description of Artificial Sequence: Synthetic peptide”
YPRSGS

SEQ ID NO: 26 - protein, 9 aa, Artificial Sequence
  source: /note=“Description of Artificial Sequence: Synthetic peptide”
RNLFLPMEY

SEQ ID NO: 27 - protein, 118 aa, Artificial Sequence
  source: /note=“Description of Artificial Sequence: Synthetic polypeptide”
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSS

SEQ ID NO: 28 - DNA, 354 bp, Artificial Sequence
  source: /note=“Description of Artificial Sequence: Synthetic polynucleotide”
gaggtgcagctggtgcagtcaggcgccgaagtgaagaagcccggcgagtcactgaagattagctgtaaagtctcaggctacaccttcaccgatcacactattcactggatgagacagatgcccggtaaaggcctggagtggatgggctatatctaccctagatcaggctctattaactataacgagaagtttaagggtcaggtcacaattagcgccgataagtctagctctaccgcctacctgcagtggtctagcctgaaggctagtgacaccgctatgtactactgcgctagacgtaacctgttcctgcctatggaatactggggtcagggcaccctggtcaccgtgtctagc

SEQ ID NO: 29 - protein, 448 aa, Artificial Sequence
  source: /note=“Description of Artificial Sequence: Synthetic polypeptide”
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 1344 bp, Artificial Sequence
  source: /note=“Description of Artificial Sequence: Synthetic polynucleotide”
gaggtgcagctggtgcagtcaggcgccgaagtgaagaagcccggcgagtcactgaagattagctgtaaagtctcaggctacaccttcaccgatcacactattcactggatgagacagatgcccggtaaaggcctggagtggatgggctatatctaccctagatcaggctctattaactataacgagaagtttaagggtcaggtcacaattagcgccgataagtctagctctaccgcctacctgcagtggtctagcctgaaggctagtgacaccgctatgtactactgcgctagacgtaacctgttcctgcctatggaatactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 31 - protein, 17 aa, Artificial Sequence
  source: /note=“Description of Artificial Sequence: Synthetic peptide”
RSSQSLLSSGDQKNYLT

SEQ ID NO: 32 - protein, 7 aa, Artificial Sequence
  source: /note=“Description of Artificial Sequence: Synthetic peptide”
WASTRES

SEQ ID NO: 33 - protein, 9 aa, Artificial Sequence
  source: /note=“Description of Artificial Sequence: Synthetic peptide”
QNDYRYPLT

SEQ ID NO: 34 - protein, 13 aa, Artificial Sequence
  source: /note=“Description of Artificial Sequence: Synthetic peptide”
SQSLLSSGDQKNY

SEQ ID NO: 35 - protein, 3 aa, Artificial Sequence
  source: /note=“Description of Artificial Sequence: Synthetic peptide”
WAS

SEQ ID NO: 36 - protein, 6 aa, Artificial Sequence
  source: /note=“Description of Artificial Sequence: Synthetic peptide”
DYRYPL

SEQ ID NO: 37 - protein, 113 aa, Artificial Sequence
  source: /note=“Description of Artificial Sequence: Synthetic polypeptide”
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEIK

SEQ ID NO: 38 - DNA, 339 bp, Artificial Sequence
  source: /note=“Description of Artificial Sequence: Synthetic polynucleotide”
gatatcgtgatgactcagacccccctgagcctgcccgtgacccctggcgagcctgcctctattagctgtagatctagtcagtcactgctgtctagcggcgatcagaagaactacctgacctggtatctgcagaagcccggtcagtcacctcagctgctgatctactgggcctctactagagaatcaggcgtgcccgataggtttagcggtagcggtagtggcaccgacttcaccctgaagatctctagggtggaagccgaggacgtgggcgtctactactgtcagaacgactatagataccccctgaccttcggtcagggcactaagctggagattaag

SEQ ID NO: 39 - protein, 220 aa, Artificial Sequence
  source: /note=“Description of Artificial Sequence: Synthetic polypeptide”
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 660 bp, Artificial Sequence
  source: /note=“Description of Artificial Sequence: Synthetic polynucleotide”
gatatcgtgatgactcagacccccctgagcctgcccgtgacccctggcgagcctgcctctattagctgtagatctagtcagtcactgctgtctagcggcgatcagaagaactacctgacctggtatctgcagaagcccggtcagtcacctcagctgctgatctactgggcctctactagagaatcaggcgtgcccgataggtttagcggtagcggtagtggcaccgacttcaccctgaagatctctagggtggaagccgaggacgtgggcgtctactactgtcagaacgactatagataccccctgaccttcggtcagggcactaagctggagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 41 - protein, 5 aa, Artificial Sequence
  source: /note=“Description of Artificial Sequence: Synthetic peptide”
DHTIH

SEQ ID NO: 42 - protein, 17 aa, Artificial Sequence
  source: /note=“Description of Artificial Sequence: Synthetic peptide”
YIYPRSGSINYNEKFKG

SEQ ID NO: 43 - protein, 9 aa, Artificial Sequence
  source: /note=“Description of Artificial Sequence: Synthetic peptide”
RNLFLPMEY

SEQ ID NO: 44 - protein, 7 aa, Artificial Sequence
  source: /note=“Description of Artificial Sequence: Synthetic peptide”
GYTFTDH

SEQ ID NO: 45 - protein, 6 aa, Artificial Sequence
  source: /note=“Description of Artificial Sequence: Synthetic peptide”
YPRSGS

SEQ ID NO: 46 - protein, 9 aa, Artificial Sequence
  source: /note=“Description of Artificial Sequence: Synthetic peptide”
RNLFLPMEY

SEQ ID NO: 47 - protein, 118 aa, Artificial Sequence; identical to SEQ ID NO: 27
  source: /note=“Description of Artificial Sequence: Synthetic polypeptide”

SEQ ID NO: 48 - DNA, 354 bp, Artificial Sequence; identical to SEQ ID NO: 28
  source: /note=“Description of Artificial Sequence: Synthetic polynucleotide”

SEQ ID NO: 49 - protein, 448 aa, Artificial Sequence; identical to SEQ ID NO: 29
  source: /note=“Description of Artificial Sequence: Synthetic polypeptide”

SEQ ID NO: 50 - DNA, 1344 bp, Artificial Sequence; identical to SEQ ID NO: 30
  source: /note=“Description of Artificial Sequence: Synthetic polynucleotide”

SEQ ID NO: 51 - protein, 17 aa, Artificial Sequence
  source: /note=“Description of Artificial Sequence: Synthetic peptide”
RSSQSLLSSGNQKNYLT

SEQ ID NO: 52 - protein, 7 aa, Artificial Sequence
  source: /note=“Description of Artificial Sequence: Synthetic peptide”
WASTRES

SEQ ID NO: 53 - protein, 9 aa, Artificial Sequence
  source: /note=“Description of Artificial Sequence: Synthetic peptide”
QNDYSYPLT

SEQ ID NO: 54 - protein, 13 aa, Artificial Sequence
  source: /note=“Description of Artificial Sequence: Synthetic peptide”
SQSLLSSGNQKNY

SEQ ID NO: 55 - protein, 3 aa, Artificial Sequence
  source: /note=“Description of Artificial Sequence: Synthetic peptide”
WAS

SEQ ID NO: 56 - protein, 6 aa, Artificial Sequence
  source: /note=“Description of Artificial Sequence: Synthetic peptide”
DYSYPL

SEQ ID NO: 57 - protein, 113 aa, Artificial Sequence
  source: /note=“Description of Artificial Sequence: Synthetic polypeptide”
DIVMTQTPLSLPVTPGEPASISCRSSQSLLSSGNQKNYLTWYLQKPGQSPQLLIYWASTRESGVPDRFSGSGSGTDFTLKISRVEAEDVGVYYCQNDYSYPLTFGQGTKLEIK

SEQ ID NO: 58 - DNA, 339 bp, Artificial Sequence
  source: /note=“Description of Artificial Sequence: Synthetic polynucleotide”
gatatcgtgatgactcagacccccctgagcctgcccgtgacccctggcgagcctgcctctattagctgtagatctagtcagtcactgctgtctagcggtaatcagaagaactacctgacctggtatctgcagaagcccggtcagtcacctcagctgctgatctactgggcctctactagagaatcaggcgtgcccgataggtttagcggtagcggtagtggcaccgacttcaccctgaagatctctagggtggaagccgaggacgtgggcgtctactactgtcagaacgactatagctaccccctgaccttcggtcagggcactaagctggagattaag

SEQ ID NO: 59 - protein, 220 aa, Artificial Sequence
  source: /note=“Description of Artificial Sequence: Synthetic polypeptide”
DIVMTQTPLSLPVTPGEPASISCRSSQSLLSSGNQKNYLTWYLQKPGQSPQLLIYWASTRESGVPDRFSGSGSGTDFTLKISRVEAEDVGVYYCQNDYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - DNA, 660 bp, Artificial Sequence
  source: /note=“Description of Artificial Sequence: Synthetic polynucleotide”
gatatcgtgatgactcagacccccctgagcctgcccgtgacccctggcgagcctgcctctattagctgtagatctagtcagtcactgctgtctagcggtaatcagaagaactacctgacctggtatctgcagaagcccggtcagtcacctcagctgctgatctactgggcctctactagagaatcaggcgtgcccgataggtttagcggtagcggtagtggcaccgacttcaccctgaagatctctagggtggaagccgaggacgtgggcgtctactactgtcagaacgactatagctaccccctgaccttcggtcagggcactaagctggagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 61 - protein, 5 aa, Artificial Sequence
  source: /note=“Description of Artificial Sequence: Synthetic peptide”
DHTLH

SEQ ID NO: 62 - protein, 17 aa, Artificial Sequence
  source: /note=“Description of Artificial Sequence: Synthetic peptide”
YIYPRSGSTKYNENFRG

SEQ ID NO: 63 - protein, 9 aa, Artificial Sequence
  source: /note=“Description of Artificial Sequence: Synthetic peptide”
RLLFLPLDY

SEQ ID NO: 64 - protein, 7 aa, Artificial Sequence
  source: /note=“Description of Artificial Sequence: Synthetic peptide”
GYTFTDH

SEQ ID NO: 65 - protein, 6 aa, Artificial Sequence
  source: /note=“Description of Artificial Sequence: Synthetic peptide”
YPRSGS

SEQ ID NO: 66 - protein, 9 aa, Artificial Sequence
  source: /note=“Description of Artificial Sequence: Synthetic peptide”
RLLFLPLDY

SEQ ID NO: 67 - protein, 118 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCARRLLFLPLDYWGQGTLVTVSS

SEQ ID NO: 68 - DNA, 354 bp, Artificial Sequence
  source: /note=“Description of Artificial Sequence: Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaggggtagagtgactatcaccgccgacactagctctagcaccgcctatatggaactgtctagcctgagatcagaggacaccgccgtctactactgcgctagacggctgctgttcctgcccctggactactggggtcagggcaccctggtcaccgtgtctagc

SEQ ID NO: 69 - protein, 448 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCARRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1344 bp, Artificial Sequence
  source: /note=“Description of Artificial Sequence: Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaggggtagagtgactatcaccgccgacactagctctagcaccgcctatatggaactgtctagcctgagatcagaggacaccgccgtctactactgcgctagacggctgctgttcctgcccctggactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 71 - protein, 17 aa, Artificial Sequence
  source: /note=“Description of Artificial Sequence: Synthetic peptide”
RSSQSLLSSGNQKSYLT

SEQ ID NO: 72 - protein, 7 aa, Artificial Sequence
  source: /note=“Description of Artificial Sequence: Synthetic peptide”
WASTRES

SEQ ID NO: 73 - protein, 9 aa, Artificial Sequence
  source: /note=“Description of Artificial Sequence: Synthetic peptide”
QNDYSYPFT

SEQ ID NO: 74 - protein, 13 aa, Artificial Sequence
  source: /note=“Description of Artificial Sequence: Synthetic peptide”
SQSLLSSGNQKSY

SEQ ID NO: 75 - protein, 3 aa, Artificial Sequence
  source: /note=“Description of Artificial Sequence: Synthetic peptide”
WAS

SEQ ID NO: 76 - protein, 6 aa, Artificial Sequence
  source: /note=“Description of Artificial Sequence: Synthetic peptide”
DYSYPF

SEQ ID NO: 77 - protein, 113 aa, Artificial Sequence
  source: /note=“Description of Artificial Sequence: Synthetic polypeptide”
EIVMTQSPATLSLSPGERATLSCRSSQSLLSSGNQKSYLTWYQQKPGQAPRLLIYWASTRESGIPARFSGSGSGTDFTLTISSLQPEDFAVYYCQNDYSYPFTFGQGTKLEIK

SEQ ID NO: 78 - DNA, 339 bp, Artificial Sequence
  source: /note=“Description of Artificial Sequence: Synthetic polynucleotide”
gagatcgtgatgactcagtcacccgctaccctgagcctgagccctggcgagagagctacactgagctgtagatctagtcagtcactgctgtctagcggtaatcagaagtcctacctgacctggtatcagcagaagcccggtcaggcccctagactgctgatctactgggcctctactagagagtcagggatccccgctaggtttagcggtagcggtagtggcaccgacttcaccctgactatctctagcctgcagcccgaggacttcgccgtctactactgtcagaacgactatagctaccccttcaccttcggtcagggcactaagctggagattaag

SEQ ID NO: 79 - protein, 220 aa, Artificial Sequence
  source: /note=“Description of Artificial Sequence: Synthetic polypeptide”
EIVMTQSPATLSLSPGERATLSCRSSQSLLSSGNQKSYLTWYQQKPGQAPRLLIYWASTRESGIPARFSGSGSGTDFTLTISSLQPEDFAVYYCQNDYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - DNA, 660 bp, Artificial Sequence
  source: /note=“Description of Artificial Sequence: Synthetic polynucleotide”
gagatcgtgatgactcagtcacccgctaccctgagcctgagccctggcgagagagctacactgagctgtagatctagtcagtcactgctgtctagcggtaatcagaagtcctacctgacctggtatcagcagaagcccggtcaggcccctagactgctgatctactgggcctctactagagagtcagggatccccgctaggtttagcggtagcggtagtggcaccgacttcaccctgactatctctagcctgcagcccgaggacttcgccgtctactactgtcagaacgactatagctaccccttcaccttcggtcagggcactaagctggagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 81 - protein, 5 aa, Artificial Sequence
  source: /note=“Description of Artificial Sequence: Synthetic peptide”
DHTLH

SEQ ID NO: 82 - protein, 17 aa, Artificial Sequence
  source: /note=“Description of Artificial Sequence: Synthetic peptide”
YIYPRSGSTKYNENFRG

SEQ ID NO: 83 - protein, 9 aa, Artificial Sequence
  source: /note=“Description of Artificial Sequence: Synthetic peptide”
RLLFLPLDY

SEQ ID NO: 84 - protein, 7 aa, Artificial Sequence
  source: /note=“Description of Artificial Sequence: Synthetic peptide”
GYTFTDH

SEQ ID NO: 85 - protein, 6 aa, Artificial Sequence
  source: /note=“Description of Artificial Sequence: Synthetic peptide”
YPRSGS

SEQ ID NO: 86 - protein, 9 aa, Artificial Sequence
  source: /note=“Description of Artificial Sequence: Synthetic peptide”
RLLFLPLDY

SEQ ID NO: 87 - protein, 118 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCVRRLLFLPLDYWGQGTLVTVSS

SEQ ID NO: 88 - DNA, 354 bp, Artificial Sequence
  source: /note=“Description of Artificial Sequence: Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaggggtagagtgactatcaccgccgacactagctctagcaccgcctatatggaactgtctagcctgagatcagaggacaccgccgtctactactgcgtcagacggctgctgttcctgcccctggactactggggtcagggcaccctggtcaccgtgtctagc

SEQ ID NO: 89 - protein, 448 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCVRRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - DNA, 1344 bp, Artificial Sequence
  source: /note=“Description of Artificial Sequence: Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaggggtagagtgactatcaccgccgacactagctctagcaccgcctatatggaactgtctagcctgagatcagaggacaccgccgtctactactgcgtcagacggctgctgttcctgcccctggactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 91 - protein, 17 aa, Artificial Sequence
  source: /note=“Description of Artificial Sequence: Synthetic peptide”
RSSQSLLSSGNQKSYLT

SEQ ID NO: 92 - protein, 7 aa, Artificial Sequence
  source: /note=“Description of Artificial Sequence: Synthetic peptide”
WASTRES

SEQ ID NO: 93 - protein, 9 aa, Artificial Sequence
  source: /note=“Description of Artificial Sequence: Synthetic peptide”
QNDYSYPFT

SEQ ID NO: 94 - protein, 13 aa, Artificial Sequence
  source: /note=“Description of Artificial Sequence: Synthetic peptide”
SQSLLSSGNQKSY

SEQ ID NO: 95 - protein, 3 aa, Artificial Sequence
  source: /note=“Description of Artificial Sequence: Synthetic peptide”
WAS

SEQ ID NO: 96 - protein, 6 aa, Artificial Sequence
  source: /note=“Description of Artificial Sequence: Synthetic peptide”
DYSYPF

SEQ ID NO: 97 - protein, 113 aa, Artificial Sequence; identical to SEQ ID NO: 77
  source: /note=“Description of Artificial Sequence: Synthetic polypeptide”

SEQ ID NO: 98 - DNA, 339 bp, Artificial Sequence; identical to SEQ ID NO: 78
  source: /note=“Description of Artificial Sequence: Synthetic polynucleotide”

SEQ ID NO: 99 - protein, 220 aa, Artificial Sequence; identical to SEQ ID NO: 79
  source: /note=“Description of Artificial Sequence: Synthetic polypeptide”

SEQ ID NO: 100 - DNA, 660 bp, Artificial Sequence; identical to SEQ ID NO: 80
  source: /note=“Description of Artificial Sequence: Synthetic polynucleotide”

SEQ ID NO: 101 - protein, 5 aa, Artificial Sequence
  source: /note=“Description of Artificial Sequence: Synthetic peptide”
DHTLH

SEQ ID NO: 102 - protein, 17 aa, Artificial Sequence
  source: /note=“Description of Artificial Sequence: Synthetic peptide”
YIYPRSGSTKYNENFKG

SEQ ID NO: 103 - protein, 9 aa, Artificial Sequence
  source: /note=“Description of Artificial Sequence: Synthetic peptide”
RLLFLPLDY

SEQ ID NO: 104 - protein, 7 aa, Artificial Sequence
  source: /note=“Description of Artificial Sequence: Synthetic peptide”
GYTFTDH

SEQ ID NO: 105 - protein, 6 aa, Artificial Sequence
  source: /note=“Description of Artificial Sequence: Synthetic peptide”
YPRSGS

SEQ ID NO: 106 - protein, 9 aa, Artificial Sequence
  source: /note=“Description of Artificial Sequence: Synthetic peptide”
RLLFLPLDY

SEQ ID NO: 107 - protein, 118 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFKGRVTITADTSSSTAYMELSSLRSEDTAVYYCVRRLLFLPLDYWGQGTLVTVSS

SEQ ID NO: 108 - DNA, 354 bp, Artificial Sequence
  source: /note=“Description of Artificial Sequence: Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaagggtagagtgactatcaccgccgacactagctctagcaccgcctatatggaactgtctagcctgagatcagaggacaccgccgtctactactgcgtcagacggctgctgttcctgcccctggactactggggtcagggcaccctggtcaccgtgtctagc

SEQ ID NO: 109 - protein, 448 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFKGRVTITADTSSSTAYMELSSLRSEDTAVYYCVRRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - DNA, 1344 bp, Artificial Sequence
  source: /note=“Description of Artificial Sequence: Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaagggtagagtgactatcaccgccgacactagctctagcaccgcctatatggaactgtctagcctgagatcagaggacaccgccgtctactactgcgtcagacggctgctgttcctgcccctggactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 111 - protein, 17 aa, Artificial Sequence
  source: /note=“Description of Artificial Sequence: Synthetic peptide”
RSSQSLLSSGNQKSYLT

SEQ ID NO: 112 - protein, 7 aa, Artificial Sequence
  source: /note=“Description of Artificial Sequence: Synthetic peptide”
WASTRES

SEQ ID NO: 113 - protein, 9 aa, Artificial Sequence
  source: /note=“Description of Artificial Sequence: Synthetic peptide”
QNDYSYPFT

SEQ ID NO: 114 - protein, 13 aa, Artificial Sequence
  source: /note=“Description of Artificial Sequence: Synthetic peptide”
SQSLLSSGNQKSY

SEQ ID NO: 115 - protein, 3 aa, Artificial Sequence
  source: /note=“Description of Artificial Sequence: Synthetic peptide”
WAS

SEQ ID NO: 116 - protein, 6 aa, Artificial Sequence
  source: /note=“Description of Artificial Sequence: Synthetic peptide”
DYSYPF

SEQ ID NO: 117 - protein, 113 aa, Artificial Sequence; identical to SEQ ID NO: 77
  source: /note=“Description of Artificial Sequence: Synthetic polypeptide”

SEQ ID NO: 118 - DNA, 339 bp, Artificial Sequence; identical to SEQ ID NO: 78
  source: /note=“Description of Artificial Sequence: Synthetic polynucleotide”

SEQ ID NO: 119 - protein, 220 aa, Artificial Sequence; identical to SEQ ID NO: 79
  source: /note=“Description of Artificial Sequence: Synthetic polypeptide”

SEQ ID NO: 120 - DNA, 660 bp, Artificial Sequence; identical to SEQ ID NO: 80
  source: /note=“Description of Artificial Sequence: Synthetic polynucleotide”

SEQ ID NO: 121-129 - skipped (9 entries)

SEQ ID NO: 130 - protein, 453 aa, Artificial Sequence
  source: /note=“Description of Artificial Sequence: Synthetic polypeptide”
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 131 - protein, 453 aa, Artificial Sequence
  source: /note=“Description of Artificial Sequence: Synthetic polypeptide”
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 213 aa, Artificial Sequence
  source: /note=“Description of Artificial Sequence: Synthetic polypeptide”
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEICRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 448 aa, Artificial Sequence
  source: /note=“Description of Artificial Sequence: Synthetic polypeptide”
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 134 - protein, 448 aa, Artificial Sequence
  source: /note=“Description of Artificial Sequence: Synthetic polypeptide”
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 220 aa, Artificial Sequence
  source: /note=“Description of Artificial Sequence: Synthetic polypeptide”
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEICRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 448 aa, Artificial Sequence; identical to SEQ ID NO: 133
  source: /note=“Description of Artificial Sequence: Synthetic polypeptide”

SEQ ID NO: 137 - protein, 448 aa, Artificial Sequence; identical to SEQ ID NO: 134
  source: /note=“Description of Artificial Sequence: Synthetic polypeptide”

SEQ ID NO: 138 - protein, 220 aa, Artificial Sequence
  source: /note=“Description of Artificial Sequence: Synthetic polypeptide”
DIVMTQTPLSLPVTPGEPASISCRSSQSLLSSGNQKNYLTWYLQKPGQSPQLLIYWASTRESGVPDRFSGSGSGTDFTLKISRVEAEDVGVYYCQNDYSYPLTFGQGTKLEICRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 448 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCARRLLFLPLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 140 - protein, 448 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCARRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 220 aa, Artificial Sequence
  source: /note=“Description of Artificial Sequence: Synthetic polypeptide”
EIVMTQSPATLSLSPGERATLSCRSSQSLLSSGNQKSYLTWYQQKPGQAPRLLIYWASTRESGIPARFSGSGSGTDFTLTISSLQPEDFAVYYCQNDYSYPFTFGQGTKLEICRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 448 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCVRRLLFLPLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 143 - protein, 448 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCVRRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 220 aa, Artificial Sequence; identical to SEQ ID NO: 141
  source: /note=“Description of Artificial Sequence: Synthetic polypeptide”

SEQ ID NO: 145 - protein, 448 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFKGRVTITADTSSSTAYMELSSLRSEDTAVYYCVRRLLFLPLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 146 - protein, 448 aa, Artificial Sequence
  source: /note=“Description of Artificial Sequence: Synthetic polypeptide”
QIQLVQSGAEVKKPGSSVKVSCKVSGYTFTDHTLHWMRQAPGQGLEWMGYIYPRSGSTKYNENFKGRVTITADTSSSTAYMELSSLRSEDTAVYYCVRRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 220 aa, Artificial Sequence; identical to SEQ ID NO: 141
  source: /note=“Description of Artificial Sequence: Synthetic polypeptide”

SEQ ID NO: 148 - protein, 331 aa, Artificial Sequence
  source: /note=“Description of Artificial Sequence: Synthetic polypeptide”
SASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 149 - protein, 331 aa, Artificial Sequence
  source: /note=“Description of Artificial Sequence: Synthetic polypeptide”
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 108 aa, Artificial Sequence
  source: /note=“Description of Artificial Sequence: Synthetic polypeptide”
CRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 369 bp, Artificial Sequence
  source: /note=“Description of Artificial Sequence: Synthetic polynucleotide”
caggtgcaattgcagcagagcggtccgggcctggtgaaaccgagccagaccctgagcctgacctgcgcgatttccggagatagcgtgagctctcagtctgctgcttggaactggattcgtcagagcccgagccgtggcctcgagtggctgggccgtatctactaccgtagcaaatggtacaacgactatgccttgagcgtgaaaagccgcattaccattaacccggatacttcgaaaaaccagtttagcctgcaactgaacagcgtgaccccggaagatacggccgtgtattattgcgcgcgtggtgaaggttacggtcgtgaaggtttcgctatctggggccaaggcaccctggtgactgttagctca

SEQ ID NO: 152 - DNA, 1359 bp, Artificial Sequence
  source: /note=“Description of Artificial Sequence: Synthetic polynucleotide”
caggtgcaattgcagcagagcggtccgggcctggtgaaaccgagccagaccctgagcctgacctgcgcgatttccggagatagcgtgagctctcagtctgctgcttggaactggattcgtcagagcccgagccgtggcctcgagtggctgggccgtatctactaccgtagcaaatggtacaacgactatgccttgagcgtgaaaagccgcattaccattaacccggatacttcgaaaaaccagtttagcctgcaactgaacagcgtgaccccggaagatacggccgtgtattattgcgcgcgtggtgaaggttacggtcgtgaaggtttcgctatctggggccaaggcaccctggtgactgttagctcagcctctacgaaaggcccaagcgtatttcccctggctccttctagtaaatcaacctcaggtggtacagcagcccttggctgcctggtcaaagactatttcccctgtccggtgaccgtctcatggaactcaggtgctttgacatctggtgtgcatacattcccagctgtgctgcaaagtagtggactgtacagcctttccagcgtggtcacggtgccaagtagctccttgggtactcagacttatatctgcaatgtgaaccacaagccctctaacacgaaggtggacaagcgcgtggagcccaaatcttgcgataagacgcatacttgtcccccatgccctgctcctgagctgttgggaggcccgtcagtgttcttgttccctccgaagcctaaggacactttgatgataagtaggacaccagaggtgacttgcgtggtggttgatgtgtcccatgaagatcccgaggtcaaatttaattggtacgtagatggtgtcgaagttcacaatgctaagactaagccaagggaagagcagtacaacagtacatatagggtagtctccgtgctgacagtcctccaccaggactggttgaacggcaaggaatacaaatgtaaggtgtcaaacaaagctctgcctgctcccattgagaaaacaatctctaaagccaaaggccagccgagagagccccaagtctacactttgcccccgagcagggaggaaatgaccaagaatcaggtgagtctgacgtgcctcgtcaaaggattttatccatgcgatattgcagttgaatgggagagcaatggccagccagagaacaactataaaaccacaccacccgtgctcgactctgatggcagcttcttcctctatagcaagctgacagtcgataaatctcgctggcagcaaggcaatgtgttctcctgctccgtcatgcacgaggctttgcataaccattatactcaaaaatctctgtccctgtcacctggtaaa

SEQ ID NO: 153 - DNA, 318 bp, Artificial Sequence
  source: /note=“Description of Artificial Sequence: Synthetic polynucleotide”
gatatccagatgacccagagcccgagcagcctgagcgccagcgtgggcgatcgcgtgaccattacctgcagagccagccagactatttctaacactctggcttggtaccagcagaaaccgggcaaagcgccgaaactattaatctacgctgcttctaacctgcaaagcggcgtgccgagccgctttagcggcagcggatccggcaccgatttcaccctgaccattagctctctgcaaccggaagactttgcgacctattattgccagcagtacctgtcttggttcacctttggccagggcacgaaagttgaaattaaa

SEQ ID NO: 154 - DNA, 639 bp, Artificial Sequence
  source: /note=“Description of Artificial Sequence: Synthetic polynucleotide”
gatatccagatgacccagagcccgagcagcctgagcgccagcgtgggcgatcgcgtgaccattacctgcagagccagccagactatttctaacactctggcttggtaccagcagaaaccgggcaaagcgccgaaactattaatctacgctgcttctaacctgcaaagcggcgtgccgagccgctttagcggcagcggatccggcaccgatttcaccctgaccattagctctctgcaaccggaagactttgcgacctattattgccagcagtacctgtcttggttcacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 155-159 - skipped (5 entries)

SEQ ID NO: 160 - protein, 4 aa, Artificial Sequence
  source: /note=“Description of Artificial Sequence: Synthetic peptide”
GFLG

SEQ ID NO: 161 - protein, 4 aa, Artificial Sequence
  source: /note=“Description of Artificial Sequence: Synthetic peptide”
GLFG

SEQ ID NO: 162 - protein, 4 aa, Artificial Sequence
  source: /note=“Description of Artificial Sequence: Synthetic peptide”
ALAL

SEQ ID NO: 163 - protein, 4 aa, Artificial Sequence
  source: /note=“Description of Artificial Sequence: Synthetic peptide”
GGGG

SEQ ID NO: 164 - protein, 4 aa, Artificial Sequence
  source: /note=“Description of Artificial Sequence: Synthetic peptide”
GFVG

SEQ ID NO: 165 - protein, 4 aa, Artificial Sequence
  source: /note=“Description of Artificial Sequence: Synthetic peptide”
GVFG

SEQ ID NO: 166 - skipped